SEQUENCE LISTING (265 sequences)

SEQ ID NO: 1 - DNA, 20 bp, synthetic construct
  misc_feature 1..20: note = Synthetic construct
  misc_feature 1..20: note = target site A or gRNA spacer
  source 1..20: mol_type = other DNA
tcacacagctaggaagtggg

SEQ ID NO: 2 - DNA, 20 bp, synthetic construct
  misc_feature 1..20: note = Synthetic construct
  misc_feature 1..20: note = target site B or gRNA spacer
  source 1..20: mol_type = other DNA
gaggctgcttggccgccggt

SEQ ID NO: 3 - DNA, 20 bp, synthetic construct
  misc_feature 1..20: note = Synthetic construct
  misc_feature 1..20: note = target site C or gRNA spacer
  source 1..20: mol_type = other DNA
gcccgctccgccctccagcg

SEQ ID NO: 4 - DNA, 20 bp, synthetic construct
  misc_feature 1..20: note = Synthetic construct
  misc_feature 1..20: note = target site D or gRNA spacer
  source 1..20: mol_type = other DNA
agcctgctttgtgcaaagca

SEQ ID NO: 5 - DNA, 22 bp, synthetic construct
  misc_feature 1..22: note = Synthetic construct
  misc_feature 1..22: note = target site E or gRNA spacer
  source 1..22: mol_type = other DNA
ccggccgatgacgcgccgcggg

SEQ ID NO: 6 - DNA, 22 bp, synthetic construct
  misc_feature 1..22: note = Synthetic construct
  misc_feature 1..22: note = target site F or gRNA spacer
  source 1..22: mol_type = other DNA
ggccggctactgcgcggcgccc

SEQ ID NO: 7 - DNA, 22 bp, synthetic construct
  misc_feature 1..22: note = Synthetic construct
  misc_feature 1..22: note = target site G or gRNA spacer
  source 1..22: mol_type = other DNA
acctctagctgctcccccacag

SEQ ID NO: 8 - DNA, 22 bp, synthetic construct
  misc_feature 1..22: note = Synthetic construct
  misc_feature 1..22: note = target site H or gRNA spacer
  source 1..22: mol_type = other DNA
ccgcccgctccgccctccagcg

SEQ ID NO: 9 - DNA, 22 bp, synthetic construct
  misc_feature 1..22: note = Synthetic construct
  misc_feature 1..22: note = target site I or gRNA spacer
  source 1..22: mol_type = other DNA
gcccgagagtccacatgctgct

SEQ ID NO: 10 - DNA, 22 bp, synthetic construct
  misc_feature 1..22: note = Synthetic construct
  misc_feature 1..22: note = target site J or gRNA spacer
  source 1..22: mol_type = other DNA
gctctccatttttgttaaatgc

SEQ ID NO: 11 - DNA, 20 bp, synthetic construct
  misc_feature 1..20: note = Synthetic construct
  misc_feature 1..20: note = target site K or gRNA spacer
  source 1..20: mol_type = other DNA
ctgctgtaaacccataccgg

SEQ ID NO: 12 - DNA, 20 bp, synthetic construct
  misc_feature 1..20: note = Synthetic construct
  misc_feature 1..20: note = target site L or gRNA spacer
  source 1..20: mol_type = other DNA
gcagagtacagatttacaca

SEQ ID NO: 13 - DNA, 20 bp, synthetic construct
  misc_feature 1..20: note = Synthetic construct
  misc_feature 1..20: note = target site M or gRNA spacer
  source 1..20: mol_type = other DNA
caagggagactgcagcctgg

SEQ ID NO: 14 - DNA, 20 bp, synthetic construct
  misc_feature 1..20: note = Synthetic construct
  misc_feature 1..20: note = target site N or gRNA spacer
  source 1..20: mol_type = other DNA
aagctgggaagttcttcctg

SEQ ID NO: 15 - DNA, 20 bp, synthetic construct
  misc_feature 1..20: note = Synthetic construct
  misc_feature 1..20: note = target site O or gRNA spacer
  source 1..20: mol_type = other DNA
atgcacgaatagtgctaagc

SEQ ID NO: 16 - DNA, 20 bp, synthetic construct
  misc_feature 1..20: note = Synthetic construct
  misc_feature 1..20: note = target site P or gRNA spacer
  source 1..20: mol_type = other DNA
tacacaaggcatccgtctcc

SEQ ID NO: 17 - DNA, 20 bp, synthetic construct
  misc_feature 1..20: note = Synthetic construct
  misc_feature 1..20: note = target site Q or gRNA spacer
  source 1..20: mol_type = other DNA
ggctgcttggccgccggtat

SEQ ID NO: 18 - DNA, 20 bp, synthetic construct
  misc_feature 1..20: note = Synthetic construct
  misc_feature 1..20: note = target site R or gRNA spacer
  source 1..20: mol_type = other DNA
tttagaagcggcgggccacc

SEQ ID NO: 19 - DNA, 20 bp, synthetic construct
  misc_feature 1..20: note = Synthetic construct
  misc_feature 1..20: note = target site S or gRNA spacer
  source 1..20: mol_type = other DNA
aattgaggctgcttggccgc

SEQ ID NO: 20 - DNA, 20 bp, synthetic construct
  misc_feature 1..20: note = Synthetic construct
  misc_feature 1..20: note = target site T or gRNA spacer
  source 1..20: mol_type = other DNA
gcaaagcacggagtgcaacc

SEQ ID NO: 21 - DNA, 21 bp, synthetic construct
  misc_feature 1..21: note = Synthetic construct
  misc_feature 1..21: note = target site U or gRNA spacer
  source 1..21: mol_type = other DNA
tgcatgggacaaccaccacga

SEQ ID NO: 22 - RNA, 20 nt, synthetic construct
  misc_feature 1..20: note = Synthetic construct
  misc_feature 1..20: note = gRNA A spacer RNA
  source 1..20: mol_type = other RNA
tcacacagctaggaagtggg

SEQ ID NO: 23 - RNA, 20 nt, synthetic construct
  misc_feature 1..20: note = Synthetic construct
  misc_feature 1..20: note = gRNA B spacer RNA
  source 1..20: mol_type = other RNA
gaggctgcttggccgccggt

SEQ ID NO: 24 - RNA, 20 nt, synthetic construct
  misc_feature 1..20: note = Synthetic construct
  misc_feature 1..20: note = gRNA C spacer RNA
  source 1..20: mol_type = other RNA
gcccgctccgccctccagcg

SEQ ID NO: 25 - RNA, 20 nt, synthetic construct
  misc_feature 1..20: note = Synthetic construct
  misc_feature 1..20: note = gRNA D spacer RNA
  source 1..20: mol_type = other RNA
agcctgctttgtgcaaagca

SEQ ID NO: 26 - RNA, 22 nt, synthetic construct
  misc_feature 1..22: note = Synthetic construct
  misc_feature 1..22: note = gRNA E spacer RNA
  source 1..22: mol_type = other RNA
ccggccgatgacgcgccgcggg

SEQ ID NO: 27 - RNA, 22 nt, synthetic construct
  misc_feature 1..22: note = Synthetic construct
  misc_feature 1..22: note = gRNA F spacer RNA
  source 1..22: mol_type = other RNA
ggccggctactgcgcggcgccc

SEQ ID NO: 28 - RNA, 22 nt, synthetic construct
  misc_feature 1..22: note = Synthetic construct
  misc_feature 1..22: note = gRNA G spacer RNA
  source 1..22: mol_type = other RNA
acctctagctgctcccccacag

SEQ ID NO: 29 - RNA, 22 nt, synthetic construct
  misc_feature 1..22: note = Synthetic construct
  misc_feature 1..22: note = gRNA H spacer RNA
  source 1..22: mol_type = other RNA
ccgcccgctccgccctccagcg

SEQ ID NO: 30 - RNA, 22 nt, synthetic construct
  misc_feature 1..22: note = Synthetic construct
  misc_feature 1..22: note = gRNA I spacer RNA
  source 1..22: mol_type = other RNA
gcccgagagtccacatgctgct

SEQ ID NO: 31 - RNA, 22 nt, synthetic construct
  misc_feature 1..22: note = Synthetic construct
  misc_feature 1..22: note = gRNA J spacer RNA
  source 1..22: mol_type = other RNA
gctctccatttttgttaaatgc

SEQ ID NO: 32 - RNA, 20 nt, synthetic construct
  misc_feature 1..20: note = Synthetic construct
  misc_feature 1..20: note = gRNA K spacer RNA
  source 1..20: mol_type = other RNA
ctgctgtaaacccataccgg

SEQ ID NO: 33 - RNA, 20 nt, synthetic construct
  misc_feature 1..20: note = Synthetic construct
  misc_feature 1..20: note = gRNA L spacer RNA
  source 1..20: mol_type = other RNA
gcagagtacagatttacaca

SEQ ID NO: 34 - RNA, 20 nt, synthetic construct
  misc_feature 1..20: note = Synthetic construct
  misc_feature 1..20: note = gRNA M spacer RNA
  source 1..20: mol_type = other RNA
caagggagactgcagcctgg

SEQ ID NO: 35 - RNA, 20 nt, synthetic construct
  misc_feature 1..20: note = Synthetic construct
  misc_feature 1..20: note = gRNA N spacer RNA
  source 1..20: mol_type = other RNA
aagctgggaagttcttcctg

SEQ ID NO: 36 - RNA, 20 nt, synthetic construct
  misc_feature 1..20: note = Synthetic construct
  misc_feature 1..20: note = gRNA O spacer RNA
  source 1..20: mol_type = other RNA
atgcacgaatagtgctaagc

SEQ ID NO: 37 - RNA, 20 nt, synthetic construct
  misc_feature 1..20: note = Synthetic construct
  misc_feature 1..20: note = gRNA P spacer RNA
  source 1..20: mol_type = other RNA
tacacaaggcatccgtctcc

SEQ ID NO: 38 - RNA, 20 nt, synthetic construct
  misc_feature 1..20: note = Synthetic construct
  misc_feature 1..20: note = gRNA Q spacer RNA
  source 1..20: mol_type = other RNA
ggctgcttggccgccggtat

SEQ ID NO: 39 - RNA, 20 nt, synthetic construct
  misc_feature 1..20: note = Synthetic construct
  misc_feature 1..20: note = gRNA R spacer RNA
  source 1..20: mol_type = other RNA
tttagaagcggcgggccacc

SEQ ID NO: 40 - RNA, 20 nt, synthetic construct
  misc_feature 1..20: note = Synthetic construct
  misc_feature 1..20: note = gRNA S spacer RNA
  source 1..20: mol_type = other RNA
aattgaggctgcttggccgc

SEQ ID NO: 41 - RNA, 20 nt, synthetic construct
  misc_feature 1..20: note = Synthetic construct
  misc_feature 1..20: note = gRNA T spacer RNA
  source 1..20: mol_type = other RNA
gcaaagcacggagtgcaacc

SEQ ID NO: 42 - RNA, 21 nt, synthetic construct
  misc_feature 1..21: note = Synthetic construct
  misc_feature 1..21: note = gRNA U spacer RNA
  source 1..21: mol_type = other RNA
tgcatgggacaaccaccacga

SEQ ID NO: 43 - DNA, 76 bp, synthetic construct
  misc_feature 1..76: note = Synthetic construct
  misc_feature 1..76: note = SaCas9 gRNA scaffold DNA
  source 1..76: mol_type = other DNA
gttttagtactctggaaacagaatctactaaaacaaggcaaaatgccgtgtttatctcgtcaacttgttggcgaga

SEQ ID NO: 44 - RNA, 76 nt, synthetic construct
  misc_feature 1..76: note = Synthetic construct
  misc_feature 1..76: note = SaCas9 gRNA scaffold
  source 1..76: mol_type = other RNA
gttttagtactctggaaacagaatctactaaaacaaggcaaaatgccgtgtttatctcgtcaacttgttggcgaga

SEQ ID NO: 45 - DNA, 86 bp, synthetic construct
  misc_feature 1..86: note = Synthetic construct
  misc_feature 1..86: note = SpCas9 gRNA scaffold DNA
  source 1..86: mol_type = other DNA
gtttaagagctatgctggaaacagcatagcaagtttaaataaggctagtccgttatcaacttgaaaaagtggcaccgagtcggtgc

SEQ ID NO: 46 - RNA, 86 nt, synthetic construct
  misc_feature 1..86: note = Synthetic construct
  misc_feature 1..86: note = SpCas9 gRNA scaffold
  source 1..86: mol_type = other RNA
gtttaagagctatgctggaaacagcatagcaagtttaaataaggctagtccgttatcaacttgaaaaagtggcaccgagtcggtgc

SEQ ID NO: 47 - RNA, 96 nt, synthetic construct
  misc_feature 1..96: note = Synthetic construct
  misc_feature 1..96: note = gRNA A
  source 1..96: mol_type = other RNA
tcacacagctaggaagtggggttttagtactctggaaacagaatctactaaaacaaggcaaaatgccgtgtttatctcgtcaacttgttggcgaga

SEQ ID NO: 48 - RNA, 96 nt, synthetic construct
  misc_feature 1..96: note = Synthetic construct
  misc_feature 1..96: note = gRNA B
  source 1..96: mol_type = other RNA
gaggctgcttggccgccggtgttttagtactctggaaacagaatctactaaaacaaggcaaaatgccgtgtttatctcgtcaacttgttggcgaga

SEQ ID NO: 49 - RNA, 96 nt, synthetic construct
  misc_feature 1..96: note = Synthetic construct
  misc_feature 1..96: note = gRNA C
  source 1..96: mol_type = other RNA
gcccgctccgccctccagcggttttagtactctggaaacagaatctactaaaacaaggcaaaatgccgtgtttatctcgtcaacttgttggcgaga

SEQ ID NO: 50 - RNA, 96 nt, synthetic construct
  misc_feature 1..96: note = Synthetic construct
  misc_feature 1..96: note = gRNA D
  source 1..96: mol_type = other RNA
agcctgctttgtgcaaagcagttttagtactctggaaacagaatctactaaaacaaggcaaaatgccgtgtttatctcgtcaacttgttggcgaga

SEQ ID NO: 51 - RNA, 98 nt, synthetic construct
  misc_feature 1..98: note = Synthetic construct
  misc_feature 1..98: note = gRNA E
  source 1..98: mol_type = other RNA
ccggccgatgacgcgccgcggggttttagtactctggaaacagaatctactaaaacaaggcaaaatgccgtgtttatctcgtcaacttgttggcgaga

SEQ ID NO: 52 - RNA, 98 nt, synthetic construct
  misc_feature 1..98: note = Synthetic construct
  misc_feature 1..98: note = gRNA F
  source 1..98: mol_type = other RNA
ggccggctactgcgcggcgcccgttttagtactctggaaacagaatctactaaaacaaggcaaaatgccgtgtttatctcgtcaacttgttggcgaga

SEQ ID NO: 53 - RNA, 98 nt, synthetic construct
  misc_feature 1..98: note = Synthetic construct
  misc_feature 1..98: note = gRNA G
  source 1..98: mol_type = other RNA
acctctagctgctcccccacaggttttagtactctggaaacagaatctactaaaacaaggcaaaatgccgtgtttatctcgtcaacttgttggcgaga

SEQ ID NO: 54 - RNA, 98 nt, synthetic construct
  misc_feature 1..98: note = Synthetic construct
  misc_feature 1..98: note = gRNA H
  source 1..98: mol_type = other RNA
ccgcccgctccgccctccagcggttttagtactctggaaacagaatctactaaaacaaggcaaaatgccgtgtttatctcgtcaacttgttggcgaga

SEQ ID NO: 55 - RNA, 98 nt, synthetic construct
  misc_feature 1..98: note = Synthetic construct
  misc_feature 1..98: note = gRNA I
  source 1..98: mol_type = other RNA
gcccgagagtccacatgctgctgttttagtactctggaaacagaatctactaaaacaaggcaaaatgccgtgtttatctcgtcaacttgttggcgaga

SEQ ID NO: 56 - RNA, 98 nt, synthetic construct
  misc_feature 1..98: note = Synthetic construct
  misc_feature 1..98: note = gRNA J
  source 1..98: mol_type = other RNA
gctctccatttttgttaaatgcgttttagtactctggaaacagaatctactaaaacaaggcaaaatgccgtgtttatctcgtcaacttgttggcgaga

SEQ ID NO: 57 - RNA, 106 nt, synthetic construct
  misc_feature 1..106: note = Synthetic construct
  misc_feature 1..106: note = gRNA K
  source 1..106: mol_type = other RNA
ctgctgtaaacccataccgggtttaagagctatgctggaaacagcatagcaagtttaaataaggctagtccgttatcaacttgaaaaagtggcaccgagtcggtgc

SEQ ID NO: 58 - RNA, 106 nt, synthetic construct
  misc_feature 1..106: note = Synthetic construct
  misc_feature 1..106: note = gRNA L
  source 1..106: mol_type = other RNA
gcagagtacagatttacacagtttaagagctatgctggaaacagcatagcaagtttaaataaggctagtccgttatcaacttgaaaaagtggcaccgagtcggtgc

SEQ ID NO: 59 - RNA, 106 nt, synthetic construct
  misc_feature 1..106: note = Synthetic construct
  misc_feature 1..106: note = gRNA M
  source 1..106: mol_type = other RNA
caagggagactgcagcctgggtttaagagctatgctggaaacagcatagcaagtttaaataaggctagtccgttatcaacttgaaaaagtggcaccgagtcggtgc

SEQ ID NO: 60 - RNA, 106 nt, synthetic construct
  misc_feature 1..106: note = Synthetic construct
  misc_feature 1..106: note = gRNA N
  source 1..106: mol_type = other RNA
aagctgggaagttcttcctggtttaagagctatgctggaaacagcatagcaagtttaaataaggctagtccgttatcaacttgaaaaagtggcaccgagtcggtgc

SEQ ID NO: 61 - RNA, 106 nt, synthetic construct
  misc_feature 1..106: note = Synthetic construct
  misc_feature 1..106: note = gRNA O
  source 1..106: mol_type = other RNA
atgcacgaatagtgctaagcgtttaagagctatgctggaaacagcatagcaagtttaaataaggctagtccgttatcaacttgaaaaagtggcaccgagtcggtgc

SEQ ID NO: 62 - RNA, 106 nt, synthetic construct
  misc_feature 1..106: note = Synthetic construct
  misc_feature 1..106: note = gRNA P
  source 1..106: mol_type = other RNA
tacacaaggcatccgtctccgtttaagagctatgctggaaacagcatagcaagtttaaataaggctagtccgttatcaacttgaaaaagtggcaccgagtcggtgc

SEQ ID NO: 63 - RNA, 106 nt, synthetic construct
  misc_feature 1..106: note = Synthetic construct
  misc_feature 1..106: note = gRNA Q
  source 1..106: mol_type = other RNA
ggctgcttggccgccggtatgtttaagagctatgctggaaacagcatagcaagtttaaataaggctagtccgttatcaacttgaaaaagtggcaccgagtcggtgc

SEQ ID NO: 64 - RNA, 106 nt, synthetic construct
  misc_feature 1..106: note = Synthetic construct
  misc_feature 1..106: note = gRNA R
  source 1..106: mol_type = other RNA
tttagaagcggcgggccaccgtttaagagctatgctggaaacagcatagcaagtttaaataaggctagtccgttatcaacttgaaaaagtggcaccgagtcggtgc

SEQ ID NO: 65 - RNA, 106 nt, synthetic construct
  misc_feature 1..106: note = Synthetic construct
  misc_feature 1..106: note = gRNA S
  source 1..106: mol_type = other RNA
aattgaggctgcttggccgcgtttaagagctatgctggaaacagcatagcaagtttaaataaggctagtccgttatcaacttgaaaaagtggcaccgagtcggtgc

SEQ ID NO: 66 - RNA, 106 nt, synthetic construct
  misc_feature 1..106: note = Synthetic construct
  misc_feature 1..106: note = gRNA T
  source 1..106: mol_type = other RNA
gcaaagcacggagtgcaaccgtttaagagctatgctggaaacagcatagcaagtttaaataaggctagtccgttatcaacttgaaaaagtggcaccgagtcggtgc

SEQ ID NO: 67 - RNA, 97 nt, synthetic construct
  misc_feature 1..97: note = Synthetic construct
  misc_feature 1..97: note = gRNA U
  source 1..97: mol_type = other RNA
tgcatgggacaaccaccacgagttttagtactctggaaacagaatctactaaaacaaggcaaaatgccgtgtttatctcgtcaacttgttggcgaga

SEQ ID NO: 68 - DNA, 13865 bp, synthetic construct
  misc_feature 1..13865: note = Synthetic construct
  misc_feature 1..13865: note = gRNA VP64-dSaCas9-NLS-VP64 vector
  source 1..13865: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaaaaaaaatctcgccaacaagttgacgagataaacacggcattttgccttgttttagtagattctgtttccagagtactaaaacagagacgatttacaaccgtctccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctctctagagatatccctagcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaccggttctagcgcgctgccaccatggactacaaagaccatgacggtgattataaagatcatgacatcgattacaaggatgacgatgacaagcacggtccgcgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaacccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaaggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaatcgctagcgcaacaaacttctctctgctgaaacaagccggagatgtcgaagagaatcctggaccgatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtgtcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaatttaaatgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtt

SEQ ID NO: 69 - DNA, 3564 bp, synthetic construct
  misc_feature 1..3564: note = Synthetic construct
  misc_feature 1..3564: note = VP64-dSaCas9-NLS-VP64 (nt)
  source 1..3564: mol_type = other DNA
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaacccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaaggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgtta

SEQ ID NO: 70 - DNA, 3156 bp, synthetic construct
  misc_feature 1..3156: note = Synthetic construct
  misc_feature 1..3156: note = dSaCas9 (nt)
  source 1..3156: mol_type = other DNA
aagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 71 - protein, 1188 aa, synthetic construct
  REGION 1..1188: note = Synthetic construct
  REGION 1..1188: note = VP64-dSaCas9-NLS-VP64 (aa)
DALDDFDLDMLGSDALDDFDLDMLGSDALDDFDLDMLGSDALDDFDLDMVNPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKASRADALDDFDLDMLGSDALDDFDLDMLGSDALDDFDLDMLGSDALDDFDLDML

SEQ ID NO: 72 - protein, 1052 aa, synthetic construct
  REGION 1..1052: note = Synthetic construct
  REGION 1..1052: note = dSaCas9 (aa)
KRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 73 - protein, 1053 aa, synthetic construct
  REGION 1..1053: note = Synthetic construct
  REGION 1..1053: note = WT Sa Cas9 (aa)
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 74 - DNA, 14820 bp, synthetic construct
  misc_feature 1..14820: note = Synthetic construct
  misc_feature 1..14820: note = gRNA VP64-dSpCas9-NLS-VP64 vector
  source 1..14820: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaaaaaaaagcaccgactcggtgccactttttcaagttgataacggactagccttatttaaacttgctatgctgtttccagcatagctcttaaacagagacgatttacaaccgtctccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctctctagagatatccctagcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaccggttctagcgcgctgccaccatggactacaaagaccatgacggtgattataaagatcatgacatcgattacaaggatgacgatgacaagcacggtccgcgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaacccaaagaagaagcggaaggtcggtatccacggagtcccagcagccgacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacaaaaggccggcggccacgaaaaaggccggccaggcaaaaaagaaaaaggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaatcgctagcgcaacaaacttctctctgctgaaacaagccggagatgtcgaagagaatcctggaccgatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtgtcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaatttaaatgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtt

SEQ ID NO: 75 - DNA, 4509 bp, synthetic construct
  misc_feature 1..4509: note = Synthetic construct
  misc_feature 1..4509: note = VP64-dSpCas9-NLS-VP64 (nt)
  source 1..4509: mol_type = other DNA
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaacccaaagaagaagcggaaggtcggtatccacggagtcccagcagccgacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacaaaaggccggcggccacgaaaaaggccggccaggcaaaaaagaaaaaggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgtta

SEQ ID NO: 76 - DNA, 4101 bp, synthetic construct
  misc_feature 1..4101: note = Synthetic construct
  misc_feature 1..4101: note = dSpCas9 (nt)
  source 1..4101: mol_type = other DNA
gacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 77 - protein, 1503 aa, synthetic construct
  REGION 1..1503: note = Synthetic construct
  REGION 1..1503: note = VP64-dSpCas9-NLS-VP64 (aa)
DALDDFDLDMLGSDALDDFDLDMLGSDALDDFDLDMLGSDALDDFDLDMVNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKASRADALDDFDLDMLGSDALDDFDLDMLGSDALDDFDLDMLGSDALDDFDLDML

SEQ ID NO: 78 - protein, 1367 aa, synthetic construct
  REGION 1..1367: note = Synthetic construct
  REGION 1..1367: note = dSpCas9 (aa)
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 79 - protein, 1368 aa, synthetic construct [sequence omitted]

SEQ ID NO: 80 - DNA, 147 bp, synthetic construct
  misc_feature 1..147: note = Synthetic construct
  misc_feature 1..147: note = VP64 1 (nt)
  source 1..147: mol_type = other DNA
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatg

SEQ ID NO: 81 - protein, 49 aa, synthetic construct
  REGION 1..49: note = Synthetic construct
  REGION 1..49: note = VP64 1 (aa)
DALDDFDLDMLGSDALDDFDLDMLGSDALDDFDLDMLGSDALDDFDLDM

SEQ ID NO: 82 - DNA, 150 bp, synthetic construct
  misc_feature 1..150: note = Synthetic construct
  misc_feature 1..150: note = VP64 2 (nt)
  source 1..150: mol_type = other DNA
gacgcgctggacgatttcgatctcgacatgctgggttctgatgccctcgatgactttgacctggatatgttgggaagcgacgcattggatgactttgatctggacatgctcggctccgatgctctggacgatttcgatctcgatatgtta

SEQ ID NO: 83 - protein, 50 aa, synthetic construct
  REGION 1..50: note = Synthetic construct
  REGION 1..50: note = VP64 2 (aa)
DALDDFDLDMLGSDALDDFDLDMLGSDALDDFDLDMLGSDALDDFDLDML

SEQ ID NO: 84 - DNA, 48 bp, synthetic construct
  misc_feature 1..48: note = Synthetic construct
  misc_feature 1..48: note = NLS (nt)
  source 1..48: mol_type = other DNA
aaaaggccggcggccacgaaaaaggccggccaggcaaaaaagaaaaag

SEQ ID NO: 85 - protein, 16 aa, synthetic construct
  REGION 1..16: note = Synthetic construct
  REGION 1..16: note = NLS (aa)
KRPAATKKAGQAKKKK

SEQ ID NO: 86 - DNA, 240 bp, synthetic construct
  misc_feature 1..240: note = Synthetic construct
  misc_feature 1..240: note = Control peptide (nt)
  source 1..240: mol_type = other DNA
ggaattgtgaccctgcctcatcgctacgtgtacctgatcaggtttcttgcggaccacgtcgatgctagccaatccctaactacgagaactcgtattgacacattcatgtgcgtcgcctgctctttcgacggcagtaattataccgcagtgttaagtccccagatactcgccatccgacgaaactcttgtgtacgagatatgcgattcgttgctagacgtgagtacaccaaggccgtgttc

SEQ ID NO: 87 - DNA, 240 bp, synthetic construct
  misc_feature 1..240: note = Synthetic construct
  misc_feature 1..240: note = DPOLA (nt)
  source 1..240: mol_type = other DNA
aagagcaagaagggcagacaggaggccctggagagactgaagaaggccaaggccggcgagaagtacaagtacgaggtggaggacttcaccggcgtgtacgaggaggtggacgaggagcagtacagcaagctggtgcaggccagacaggacgacgactggatcgtggacgacgacggcatcggctacgtggaggacggcagagagatcttcgacgacgacctggaggacgacgccctggac

SEQ ID NO: 88 - DNA, 240 bp, synthetic construct
  misc_feature 1..240: note = Synthetic construct
  misc_feature 1..240: note = ENL (nt)
  source 1..240: mol_type = other DNA
agcaagcccgagaagatcctgaagaagggcacctacgacaaggcctacaccgacgagctggtggagctgcacagaagactgatggccctgagagagagaaacgtgctgcagcagatcgtgaacctgatcgaggagaccggccacttcaacgtgaccaacaccaccttcgacttcgacctgttcagcctggacgagaccaccgtgagaaagctgcagagctgcctggaggccgtggccacc

SEQ ID NO: 89 - DNA, 240 bp, synthetic construct
  misc_feature 1..240: note = Synthetic construct
  misc_feature 1..240: note = FOXO3 (nt)
  source 1..240: mol_type = other DNA
gacagcctgagcggcagcagcctgtacagcaccagcgccaacctgcccgtgatgggccacgagaagttccccagcgacctggacctggacatgttcaacggcagcctggagtgcgacatggagagcatcatcagaagcgagctgatggacgccgacggcctggacttcaacttcgacagcctgatcagcacccagaacgtggtgggcctgaacgtgggcaacttcaccggcgccaagcag

SEQ ID NO: 90 - DNA, 240 bp, synthetic construct
  misc_feature 1..240: note = Synthetic construct
  misc_feature 1..240: note = HSH2D (nt)
  source 1..240: mol_type = other DNA
cccgagtggttccacggcgccatcagcagagaggacgccgagaacctgctggagagccagcccctgggcagcttcctgatcagagtgagccacagccacgtgggctacaccctgagctacaaggcccagagcagctgctgccacttcatggtgaagctgctggacgacggcaccttcatgatccccggcgagaaggtggcccacaccagcctggacgccctggtgaccttccaccagcag

SEQ ID NO: 91 - DNA, 240 bp, synthetic construct
  misc_feature 1..240: note = Synthetic construct
  misc_feature 1..240: note = NCOA2 (nt)
  source 1..240: mol_type = other DNA
cccttcggcagcagccccgacgacctgctgtgcccccaccccgctgctgaaagccccagcgacgagggcgctctgctggaccagctgtacctggccctgagaaacttcgacggcctggaggagatcgacagagccctgggcatccccgagctggtgagccagagccaggccgtggaccccgagcagttcagcagccaggacagcaacatcatgctggagcagaaggcccccgtgttcccc

SEQ ID NO: 92 - DNA, 240 bp, synthetic construct
  misc_feature 1..240: note = Synthetic construct
  misc_feature 1..240: note = NCOA3 (nt)
  source 1..240: mol_type = other DNA
ctgagaaacagcctggacgacctggtgggcccccccagcaacctggagggccagagcgacgagagagccctgctggaccagctgcacaccctgctgagcaacaccgacgccaccggcctggaggagatcgacagagccctgggcatccccgagctggtgaaccagggccaggccctggagcccaagcaggacgccttccagggccaggaggctgccgtgatgatggaccagaaggccggc

SEQ ID NO: 93 - DNA, 240 bp, synthetic construct
  misc_feature 1..240: note = Synthetic construct
  misc_feature 1..240: note = PSA1 (nt)
  source 1..240: mol_type = other DNA
ttcagaaaccagtacgacaacgacgtgaccgtgtggagcccccagggcagaatccaccagatcgagtacgccatggaggccgtgaagcagggcagcgccaccgtgggcctgaagagcaagacccacgccgtgctggtggccctgaagagagcccagagcgagctggccgcccaccagaagaagatcctgcacgtggacaaccacatcggcatcagcatcgccggcctgaccgccgacgcc

SEQ ID NO: 94 - DNA, 240 bp, synthetic construct
  misc_feature 1..240: note = Synthetic construct
  misc_feature 1..240: note = PYGO1 (nt)
  source 1..240: mol_type = other DNA
agacacggccacagcagcagcgaccccgtgtacccctgcggcatctgcaccaacgaggtgaacgacgaccaggacgccatcctgtgcgaggccagctgccagaagtggttccacagaatctgcaccggcatgaccgagaccgcctacggcctgctgaccgccgaggccagcgctgtgtggggatgcgacacctgcatggccgacaaggacgtgcagctgatgagaaccagagagaccttc

SEQ ID NO: 95 - DNA, 240 bp, synthetic construct
  misc_feature 1..240: note = Synthetic construct
  misc_feature 1..240: note = RBM39 (nt)
  source 1..240: mol_type = other DNA
ggccccatgagactgtacgtgggcagcctgcacttcaacatcaccgaggacatgctgagaggcatcttcgagcccttcggcagaatcgagagcatccagctgatgatggacagcgagaccggcagaagcaagggctacggcttcatcaccttcagcgacagcgagtgcgccaagaaggccctggagcagctgaacggcttcgagctggccggcagacccatgaaggtgggccacgtgacc

SEQ ID NO: 96 - DNA, 240 bp, synthetic construct
  misc_feature 1..240: note = Synthetic construct
  misc_feature 1..240: note = HERC2 (nt)
  source 1..240: mol_type = other DNA
accctgatcagaaaggccgacctggagaaccacaacaaggacggcggcttctggaccgtgatcgacggcaaggtgtacgacatcaaggacttccagacccagagcctgaccggcaacagcatcctggcccagttcgccggcgaggaccccgtggtggccctggaagctgctctgcagttcgaggacaccagagagagcatgcacgccttctgcgtgggccagtacctggagcccgaccag

SEQ ID NO: 97 - DNA, 240 bp, synthetic construct
  misc_feature 1..240: note = Synthetic construct
  misc_feature 1..240: note = DMD control (nt)
  source 1..240: mol_type = other DNA
ccctactacatcaaccacgagacccagaccacctgctgggaccaccccaagatgaccgagctgtaccagagcctggccgacctgaacaacgtgagattcagcgcctacaggaccgccatgaagctgagaagactgcagaaggccctgtgcctggacctgctgagcctgagcgccgcctgcgacgccttggaccagcacaacctgaagcagaacgaccagcccatggacatcctgcagatc

SEQ ID NO: 98 - DNA, 240 bp, synthetic construct
  misc_feature 1..240: note = Synthetic construct
  misc_feature 1..240: note = NOTCH2 (nt)
  source 1..240: mol_type = other DNA
aacagatacagctgcgtgtgcagccccggcttcaccggccagagatgcaacatcgacatcgacgagtgcgccagcaacccctgcagaaagggcgccacctgcatcaacggcgtgaacggcttcagatgcatctgccccgagggcccccaccaccccagctgctacagccaggtgaacgagtgcctgagcaacccctgcatccacggcaactgcaccggcggcctgagcggctacaagtgc

SEQ ID NO: 99 - protein, 80 aa, synthetic construct
  REGION 1..80: note = Synthetic construct
  REGION 1..80: note = Control peptide (aa)
GIVTLPHRYVYLIRFLADHVDASQSLTTRTRIDTFMCVACSFDGSNYTAVLSPQILAIRRNSCVRDMRFVARREYTKAVF

SEQ ID NO: 100 - protein, 80 aa, synthetic construct
  REGION 1..80: note = Synthetic construct
  REGION 1..80: note = DPOLA (aa)
KSKKGRQEALERLKKAKAGEKYKYEVEDFTGVYEEVDEEQYSKLVQARQDDDWIVDDDGIGYVEDGREIFDDDLEDDALD

SEQ ID NO: 101 - protein, 80 aa, synthetic construct
  REGION 1..80: note = Synthetic construct
  REGION 1..80: note = ENL (aa)
SKPEKILKKGTYDKAYTDELVELHRRLMALRERNVLQQIVNLIEETGHFNVTNTTFDFDLFSLDETTVRKLQSCLEAVAT

SEQ ID NO: 102 - protein, 80 aa, synthetic construct
  REGION 1..80: note = Synthetic construct
  REGION 1..80: note = FOXO3 (aa)
DSLSGSSLYSTSANLPVMGHEKFPSDLDLDMFNGSLECDMESIIRSELMDADGLDFNFDSLISTQNVVGLNVGNFTGAKQ

SEQ ID NO: 103 - protein, 80 aa, synthetic construct
  REGION 1..80: note = Synthetic construct
  REGION 1..80: note = HSH2D (aa)
PEWFHGAISREDAENLLESQPLGSFLIRVSHSHVGYTLSYKAQSSCCHFMVKLLDDGTFMIPGEKVAHTSLDALVTFHQQ

SEQ ID NO: 104 - protein, 80 aa, synthetic construct
  REGION 1..80: note = Synthetic construct
  REGION 1..80: note = NCOA2 (aa)
PFGSSPDDLLCPHPAAESPSDEGALLDQLYLALRNFDGLEEIDRALGIPELVSQSQAVDPEQFSSQDSNIMLEQKAPVFP

SEQ ID NO: 105 - protein, 80 aa, synthetic construct
  REGION 1..80: note = Synthetic construct
  REGION 1..80: note = NCOA3 (aa)
LRNSLDDLVGPPSNLEGQSDERALLDQLHTLLSNTDATGLEEIDRALGIPELVNQGQALEPKQDAFQGQEAAVMMDQKAG

SEQ ID NO: 106 - protein, 80 aa, synthetic construct
  REGION 1..80: note = Synthetic construct
  REGION 1..80: note = PSA1 (aa)
FRNQYDNDVTVWSPQGRIHQIEYAMEAVKQGSATVGLKSKTHAVLVALKRAQSELAAHQKKILHVDNHIGISIAGLTADA

SEQ ID NO: 107 - protein, 80 aa, synthetic construct
  REGION 1..80: note = Synthetic construct
  REGION 1..80: note = PYGO1 (aa)
RHGHSSSDPVYPCGICTNEVNDDQDAILCEASCQKWFHRICTGMTETAYGLLTAEASAVWGCDTCMADKDVQLMRTRETF

SEQ ID NO: 108 - protein, 80 aa, synthetic construct
  REGION 1..80: note = Synthetic construct
  REGION 1..80: note = RBM39 (aa)
GPMRLYVGSLHFNITEDMLRGIFEPFGRIESIQLMMDSETGRSKGYGFITFSDSECAKKALEQLNGFELAGRPMKVGHVT

SEQ ID NO: 109 - protein, 80 aa, synthetic construct
  REGION 1..80: note = Synthetic construct
  REGION 1..80: note = HERC2 (aa)
TLIRKADLENHNKDGGFWTVIDGKVYDIKDFQTQSLTGNSILAQFAGEDPVVALEAALQFEDTRESMHAFCVGQYLEPDQ

SEQ ID NO: 110 - protein, 80 aa, synthetic construct
  REGION 1..80: note = Synthetic construct
  REGION 1..80: note = DMD (aa)
PYYINHETQTTCWDHPKMTELYQSLADLNNVRFSAYRTAMKLRRLQKALCLDLLSLSAACDALDQHNLKQNDQPMDILQI

SEQ ID NO: 111 - protein, 80 aa, synthetic construct
  REGION 1..80: note = Synthetic construct
  REGION 1..80: note = NOTCH2 (aa)
NRYSCVCSPGFTGQRCNIDIDECASNPCRKGATCINGVNGFRCICPEGPHHPSCYSQVNECLSNPCIHGNCTGGLSGYKC

SEQ ID NO: 112 - protein, 617 aa, synthetic construct
  REGION 1..617: note = Synthetic construct
  REGION 1..617: note = p300 core (aa)
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 113 - protein, 1468 aa, Homo sapiens
  REGION 1..1468: note = DNA polymerase alpha catalytic subunit (DPOLA)isoform 1
MAPVHGDDCEIGASALSDSGSFVSSRARREKKSKKGRQEALERLKKAKAGEKYKYEVEDFTGVYEEVDEEQYSKLVQARQDDDWIVDDDGIGYVEDGREIFDDDLEDDALDADEKGKDGKARNKDKRNVKKLAVTKPNNIKSMFIACAGKKTADKAVDLSKDGLLGDILQDLNTETPQITPPPVMILKKKRSIGASPNPFSVHTATAVPSGKIASPVSRKEPPLTPVPLKRAEFAGDDVQVESTEEEQESGAMEFEDGDFDEPMEVEEVDLEPMAAKAWDKESEPAEEVKQEADSGKGTVSYLGSFLPDVSCWDIDQEGDSSFSVQEVQVDSSHLPLVKGADEEQVFHFYWLDAYEDQYNQPGVVFLFGKVWIESAETHVSCCVMVKNIERTLYFLPREMKIDLNTGKETGTPISMKDVYEEFDEKIATKYKIMKFKSKPVEKNYAFEIPDVPEKSEYLEVKYSAEMPQLPQDLKGETFSHVFGTNTSSLELFLMNRKIKGPCWLEVKSPQLLNQPVSWCKVEAMALKPDLVNVIKDVSPPPLVVMAFSMKTMQNAKNHQNEIIAMAALVHHSFALDKAAPKPPFQSHFCVVSKPKDCIFPYAFKEVIEKKNVKVEVAATERTLLGFFLAKVHKIDPDIIVGHNIYGFELEVLLQRINVCKAPHWSKIGRLKRSNMPKLGGRSGFGERNATCGRMICDVEISAKELIRCKSYHLSELVQQILKTERVVIPMENIQNMYSESSQLLYLLEHTWKDAKFILQIMCELNVLPLALQITNIAGNIMSRTLMGGRSERNEFLLLHAFYENNYIVPDKQIFRKPQQKLGDEDEEIDGDTNKYKKGRKKAAYAGGLVLDPKVGFYDKFILLLDFNSLYPSIIQEFNICFTTVQRVASEAQKVTEDGEQEQIPELPDPSLEMGILPREIRKLVERRKQVKQLMKQQDLNPDLILQYDIRQKALKLTANSMYGCLGFSYSRFYAKPLAALVTYKGREILMHTKEMVQKMNLEVIYGDTDSIMINTNSTNLEEVFKLGNKVKSEVNKLYKLLEIDIDGVFKSLLLLKKKKYAALVVEPTSDGNYVTKQELKGLDIVRRDWCDLAKDTGNFVIGQILSDQSRDTIVENIQKRLIEIGENVLNGSVPVSQFEINKALTKDPQDYPDKKSLPHVHVALWINSQGGRKVKAGDTVSYVICQDGSNLTASQRAYAPEQLQKQDNLTIDTQYYLAQQIHPVVARICEPIDGIDAVLIATWLGLDPTQFRVHHYHKDEENDALLGGPAQLTDEEKYRDCERFKCPCPTCGTENIYDNVFDGSGTDMEPSLYRCSNIDCKASPLTFTVQLSNKLIMDIRRFIKKYYDGWLICEEPTCRNRTRHLPLQFSRTGPLCPACMKATLQPEYSDKSLYTQLCFYRYIFDAECALEKLTTDHEKDKLKKQFFTPKVLQDYRKLKNTAEQFLSRSGYSEVNLSKLFAGCAVKS

SEQ ID NO: 114 - protein, 600 aa, Homo sapiens
  REGION 1..600: note = protein ENL (ENL) isoform X1
MCAFPAFPQFWDSEIPGQRHQLRSSFCVCVCTCAPVCRLDSDFKRCTVQVRLELGHRAQLRKKPTTEGFTHDWMVFVRGPEQCDIQHFVEKVVFWLHDSFPKPRRVCKEPPYKVEESGYAGFIMPIEVHFKNKEEPRKVCFTYDLFLNLEGNPPVNHLRCEKLTFNNPTTEFRYKLLRAGGVMVMPEGADTVSRPSPDYPMLPTIPLSAFSDPKKTKPSHGSKDANKESSKTSKPHKVTKEHRERPRKDSESKSSSKELEREQAKSSKDTSRKLGEGRLPKEEKAPPPKAAFKEPKMALKETKLESTSPKGGPPPPPPPPPRASSKRPATADSPKPSAKKQKKSSSKGSRSAPGTSPRTSSSSSFSDKKPAKDKSSTRGEKVKAESEPREAKKALEVEESNSEDEASFKSESAQSSPSNSSSSSDSSSDSDFEPSQNHSQGPLRSMVEDLQSEESDEDDSSSGEEAAGKTNPGRDSRLSFSDSESDNSADSSLPSREPPPPQKPPPPNSKVSGRRSPESCSKPEKILKKGTYDKAYTDELVELHRRLMALRERNVLQQIVNLIEETGHFNVTNTTFDFDLFSLDETTVRKLQSCLEAVAT

SEQ ID NO: 115 - protein, 673 aa, Homo sapiens
  REGION 1..673: note = forkhead box protein O3 (FOXO3)
MAEAPASPAPLSPLEVELDPEFEPQSRPRSCTWPLQRPELQASPAKPSGETAADSMIPEEEDDEDDEDGGGRAGSAMAIGGGGGSGTLGSGLLLEDSARVLAPGGQDPGSGPATAAGGLSGGTQALLQPQQPLPPPQPGAAGGSGQPRKCSSRRNAWGNLSYADLITRAIESSPDKRLTLSQIYEWMVRCVPYFKDKGDSNSSAGWKNSIRHNLSLHSRFMRVQNEGTGKSSWWIINPDGGKSGKAPRRRAVSMDNSNKYTKSRGRAAKKKAALQTAPESADDSPSQLSKWPGSPTSRSSDELDAWTDFRSRTNSNASTVSGRLSPIMASTELDEVQDDDAPLSPMLYSSSASLSPSVSKPCTVELPRLTDMAGTMNLNDGLTENLMDDLLDNITLPPSQPSPTGGLMQRSSSFPYTTKGSGLGSPTSSFNSTVFGPSSLNSLRQSPMQTIQENKPATFSSMSHYGNQTLQDLLTSDSLSHSDVMMTQSDPLMSQASTAVSAQNSRRNVMLRNDPMMSFAAQPNQGSLVNQNLLHHQHQTQGALGGSRALSNSVSNMGLSESSSLGSAKHQQQSPVSQSMQTLSDSLSGSSLYSTSANLPVMGHEKFPSDLDLDMFNGSLECDMESIIRSELMDADGLDFNFDSLISTQNVVGLNVGNFTGAKQASSQSWVPG

SEQ ID NO: 116 - protein, 352 aa, Homo sapiens
  REGION 1..352: note = hematopoietic SH2 domain-containing protein(HSH2D) isoform 1
MTEAGKLPLPLPPRLDWFVHTQMGQLAQDGVPEWFHGAISREDAENLLESQPLGSFLIRVSHSHVGYTLSYKAQSSCCHFMVKLLDDGTFMIPGEKVAHTSLDALVTFHQQKPIEPRRELLTQPCRQKDPANVDYEDLFLYSNAVAEEAACPVSAPEEASPKPVLCHQSKERKPSAEMNRITTKEATSSCPPKSPLGETRQKLWRSLKMLPERGQRVRQQLKSHLATVNLSSLLDVRRSTVISGPGTGKGSQDHSGDPTSGDRGYTDPCVATSLKSPSQPQAPKDRKVPTRKAERSVSCIEVTPGDRSWHQMVVRALSSQESKPEHQGLAEPENDQLPEEYQQPPPFAPGYC

SEQ ID NO: 117 - protein, 1533 aa, Homo sapiens
  REGION 1..1533: note = nuclear receptor coactivator 2 (NCOA2) isoform X1
MSGMGENTSDPSRAETRKRKECPDQLGPSPKRNTEKRNREQENKYIEELAELIFANFNDIDNFNFKPDKCAILKETVKQIRQIKEQEKAAAANIDEVQKSDVSSTGQGVIDKDALGPMMLEALDGFFFVVNLEGNVVFVSENVTQYLRYNQEELMNKSVYSILHVGDHTEFVKNLLPKSIVNGGSWSGEPPRRNSHTFNCRMLVKPLPDSEEEGHDNQEAHQKYETMQCFAVSQPKSIKEEGEDLQSCLICVARRVPMKERPVLPSSESFTTRQDLQGKITSLDTSTMRAAMKPGWEDLVRRCIQKFHAQHEGESVSYAKRHHHEVLRQGLAFSQIYRFSLSDGTLVAAQTKSKLIRSQTTNEPQLVISLHMLHREQNVCVMNPDLTGQTMGKPLNPISSNSPAHQALCSGNPGQDMTLSSNINFPINGPKEQMGMPMGRFGGSGGMNHVSGMQATTPQGSNYALKMNSPSQSSPGMNPGQPTSMLSPRHRMSPGVAGSPRIPPSQFSPAGSLHSPVGVCSSTGNSHSYTNSSLNALQALSEGHGVSLGSSLASPDLKMGNLQNSPVNMNPPPLSKMGSLDSKDCFGLYGEPSEGTTGQAESSCHPGEQKETNDPNLPPAVSSERADGQSRLHDSKGQTKLLQLLTTKSDQMEPSPLASSLSDTNKDSTGSLPGSGSTHGTSLKEKHKILHRLLQDSSSPVDLAKLTAEATGKDLSQESSSTAPGSEVTIKQEPVSPKKKENALLRYLLDKDDTKDIGLPEITPKLERLDSKTDPASNTKLIAMKTEKEEMSFEPGDQPGSELDNLEEILDDLQNSQLPQLFPDTRPGAPAGSVDKQAIINDLMQLTAENSPVTPVGAQKTALRISQSTFNNPRPGQLGRLLPNQNLPLDITLQSPTGAGPFPPIRNSSPYSVIPQPGMMGNQGMIGNQGNLGNSSTGMIGNSASRPTMPSGEWAPQSSAVRVTCAATTSAMNRPVQGGMIRNPAASIPMRPSSQPGQRQTLQSQVMNIGPSELEMNMGGPQYSQQQAPPNQTAPWPESILPIDQASFASQNRQPFGSSPDDLLCPHPAAESPSDEGALLDQLYLALRNFDGLEEIDRALGIPELVSQSQAVDPEQFSSQDSNIMLEQKAPVFPQQYASQAQMAQGSYSPMQDPNFHTMGQRPSYATLRMQPRPGLRPTGLVQNQPNQLRLQLQHRLQAQQNRQPLMNQISNVSNVNLTLRPGVPTQAPINAQMLAQRQREILNQHLRQRQMHQQQQVQQRTLMMRGQGLNMTPSMVAPSGMPATMSNPRIPQANAQQFPFPPNYGTGLRSPPPFTSPFSPVSPSVGSQLLSHSSLHGSQMNLANQGMIGNLGGQLGPVRSPQVQHSTFQALSSGISQQPDPGFTGATTPQSPLMSPRMAHTQSPMMQQSQANPAYQAPSDINGWAQGNMGGNSMFSQQSPPHFGQQANTSMYSNNMNINVSMATNTGGMSSMNQMTGQISMTSVTSVPTSGLSSMGPEQVNDPALRGGNLFPNQLPGMDMIKQEGDTTRKYC

SEQ ID NO: 118 - protein, 1424 aa, Homo sapiens
  REGION 1..1424: note = nuclear receptor coactivator 3 (NCOA3) isoform a
MSGLGENLDPLASDSRKRKLPCDTPGQGLTCSGEKRRREQESKYIEELAELISANLSDIDNFNVKPDKCAILKETVRQIRQIKEQGKTISNDDDVQKADVSSTGQGVIDKDSLGPLLLQALDGFLFVVNRDGNIVFVSENVTQYLQYKQEDLVNTSVYNILHEEDRKDFLKNLPKSTVNGVSWTNETQRQKSHTFNCRMLMKTPHDILEDINASPEMRQRYETMQCFALSQPRAMMEEGEDLQSCMICVARRITTGERTFPSNPESFITRHDLSGKVVNIDTNSLRSSMRPGFEDIIRRCIQRFFSLNDGQSWSQKRHYQEAYLNGHAETPVYRFSLADGTIVTAQTKSKLFRNPVTNDRHGFVSTHFLQREQNGYRPNPNPVGQGIRPPMAGCNSSVGGMSMSPNQGLQMPSSRAYGLADPSTTGQMSGARYGGSSNIASLTPGPGMQSPSSYQNNNYGLNMSSPPHGSPGLAPNQQNIMISPRNRGSPKIASHQFSPVAGVHSPMASSGNTGNHSFSSSSLSALQAISEGVGTSLLSTLSSPGPKLDNSPNMNITQPSKVSNQDSKSPLGFYCDQNPVESSMCQSNSRDHLSDKESKESSVEGAENQRGPLESKGHKKLLQLLTCSSDDRGHSSLTNSPLDSSCKESSVSVTSPSGVSSSTSGGVSSTSNMHGSLLQEKHRILHKLLQNGNSPAEVAKITAEATGKDTSSITSCGDGNVVKQEQLSPKKKENNALLRYLLDRDDPSDALSKELQPQVEGVDNKMSQCTSSTIPSSSQEKDPKIKTETSEEGSGDLDNLDAILGDLTSSDFYNNSISSNGSHLGTKQQVFQGTNSLGLKSSQSVQSIRPPYNRAVSLDSPVSVGSSPPVKNISAFPMLPKQPMLGGNPRMMDSQENYGSSMGGPNRNVTVTQTPSSGDWGLPNSKAGRMEPMNSNSMGRPGGDYNTSLPRPALGGSIPTLPLRSNSIPGARPVLQQQQQMLQMRPGEIPMGMGANPYGQAAASNQLGSWPDGMLSMEQVSHGTQNRPLLRNSLDDLVGPPSNLEGQSDERALLDQLHTLLSNTDATGLEEIDRALGIPELVNQGQALEPKQDAFQGQEAAVMMDQKAGLYGQTYPAQGPPMQGGFHLQGQSPSFNSMMNQMNQQGNFPLQGMHPRANIMRPRTNTPKQLRMQLQQRLQGQQFLNQSRQALELKMENPTAGGAAVMRPMMQPQVSSQQGFLNAQMVAQRSRELLSHHFRQQRVAMMMQQQQQQQQQQQQQQQQQQQQQQQQQQQQQTQAFSPPPNVTASPSMDGLLAGPTMPQAPPQQFPYQPNYGMGQQPDPAFGRVSSPPNAMMSSRMGPSQNPMMQHPQAASIYQSSEMKGWPSGNLARNSSFSQQQFAHQGNPAVYSMVHMNGSSGHMGQMNMNPMPMSGMPMGPDQKYC

SEQ ID NO: 119 - protein, 269 aa, Homo sapiens
  REGION 1..269: note = proteasome subunit alpha type-1 (PSA1) isoform 1
MQLSKVKFRNQYDNDVTVWSPQGRIHQIEYAMEAVKQGSATVGLKSKTHAVLVALKRAQSELAAHQKKILHVDNHIGISIAGLTADARLLCNFMRQECLDSRFVFDRPLPVSRLVSLIGSKTQIPTQRYGRRPYGVGLLIAGYDDMGPHIFQTCPSANYFDCRAMSIGARSQSARTYLERHMSEFMECNLNELVKHGLRALRETLPAEQDLTTKNVSIGIVGKDLEFTIYDDDDVSPFLEGLEERPQRKAQPAQPADEPAEKADEPMEH

SEQ ID NO: 120 - protein, 419 aa, Homo sapiens
  REGION 1..419: note = pygopus homolog 1 (PYGO1) isoform 1
MPAENSPAPAYKVSSHGGDSGLDGLGGPGVQLGSPDKKKRKANTQGPSFPPLSEYAPPPNPNSDHLVAANPFDDNYNTISYKPLPSSNPYLGPGYPGFGGYSTFRMPPHVPPRMSSPYCGPYSLRNQPHPFPQNPLGMGFNRPHAFNFGPHDNSSFGNPSYNNALSQNVNMPNQHFRQNPAENFSQIPPQNASQVSNPDLASNFVPGNNSNFTSPLESNHSFIPPPNTFGQAKAPPPKQDFTQGATKNTNQNSSAHPPHLNMDDTVNQSNIELKNVNRNNAVNQENSRSSSTEATNNNPANGTQNKPRQPRGAADACTTEKSNKSSLHPNRHGHSSSDPVYPCGICTNEVNDDQDAILCEASCQKWFHRICTGMTETAYGLLTAEASAVWGCDTCMADKDVQLMRTRETFGPSAVGSDA

SEQ ID NO: 121 - protein, 530 aa, Homo sapiens
  REGION 1..530: note = RNA-binding protein 39 (RBM39) isoform a
MADDIDIEAMLEAPYKKDENKLSSANGHEERSKKRKKSKSRSRSHERKRSKSKERKRSRDRERKKSKSRERKRSRSKERRRSRSRSRDRRFRGRYRSPYSGPKFNSAIRGKIGLPHSIKLSRRRSRSKSPFRKDKSPVREPIDNLTPEERDARTVFCMQLAARIRPRDLEEFFSTVGKVRDVRMISDRNSRRSKGIAYVEFVDVSSVPLAIGLTGQRVLGVPIIVQASQAEKNRAAAMANNLQKGSAGPMRLYVGSLHFNITEDMLRGIFEPFGRIESIQLMMDSETGRSKGYGFITFSDSECAKKALEQLNGFELAGRPMKVGHVTERTDASSASSFLDSDELERTGIDLGTTGRLQLMARLAEGTGLQIPPAAQQALQMSGSLAFGAVAEFSFVIDLQTRLSQQTEASALAAAASVQPLATQCFQLSNMFNPQTEEEVGWDTEIKDDVIEECNKHGGVIHIYVDKNSAQGNVYVKCPSIAAAIAAVNALHGRWFAGKMITAAYVPLPTYHNLFPDSMTATQLLVPSRR

SEQ ID NO: 122 - protein, 4834 aa, Homo sapiens
  REGION 1..4834: note = E3 ubiquitin-protein ligase HERC2 (HERC2)
MPSESFCLAAQARLDSKWLKTDIQLAFTRDGLCGLWNEMVKDGEIVYTGTESTQNGELPPRKDDSVEPSGTKKEDLNDKEKKDEEETPAPIYRAKSILDSWVWGKQPDVNELKECLSVLVKEQQALAVQSATTTLSALRLKQRLVILERYFIALNRTVFQENVKVKWKSSGISLPPVDKKSSRPAGKGVEGLARVGSRAALSFAFAFLRRAWRSGEDADLCSELLQESLDALRALPEASLFDESTVSSVWLEVVERATRFLRSVVTGDVHGTPATKGPGSIPLQDQHLALAILLELAVQRGTLSQMLSAILLLLQLWDSGAQETDNERSAQGTSAPLLPLLQRFQSIICRKDAPHSEGDMHLLSGPLSPNESFLRYLTLPQDNELAIDLRQTAVVVMAHLDRLATPCMPPLCSSPTSHKGSLQEVIGWGLIGWKYYANVIGPIQCEGLANLGVTQIACAEKRFLILSRNGRVYTQAYNSDTLAPQLVQGLASRNIVKIAAHSDGHHYLALAATGEVYSWGCGDGGRLGHGDTVPLEEPKVISAFSGKQAGKHVVHIACGSTYSAAITAEGELYTWGRGNYGRLGHGSSEDEAIPMLVAGLKGLKVIDVACGSGDAQTLAVTENGQVWSWGDGDYGKLGRGGSDGCKTPKLIEKLQDLDVVKVRCGSQFSIALTKDGQVYSWGKGDNQRLGHGTEEHVRYPKLLEGLQGKKVIDVAAGSTHCLALTEDSEVHSWGSNDQCQHFDTLRVTKPEPAALPGLDTKHIVGIACGPAQSFAWSSCSEWSIGLRVPFVVDICSMTFEQLDLLLRQVSEGMDGSADWPPPQEKECVAVATLNLLRLQLHAAISHQVDPEFLGLGLGSILLNSLKQTVVTLASSAGVLSTVQSAAQAVLQSGWSVLLPTAEERARALSALLPCAVSGNEVNISPGRRFMIDLLVGSLMADGGLESALHAAITAEIQDIEAKKEAQKEKEIDEQEANASTFHRSRTPLDKDLINTGICESSGKQCLPLVQLIQQLLRNIASQTVARLKDVARRISSCLDFEQHSRERSASLDLLLRFQRLLISKLYPGESIGQTSDISSPELMGVGSLLKKYTALLCTHIGDILPVAASIASTSWRHFAEVAYIVEGDFTGVLLPELVVSIVLLLSKNAGLMQEAGAVPLLGGLLEHLDRFNHLAPGKERDDHEELAWPGIMESFFTGQNCRNNEEVTLIRKADLENHNKDGGFWTVIDGKVYDIKDFQTQSLTGNSILAQFAGEDPVVALEAALQFEDTRESMHAFCVGQYLEPDQEIVTIPDLGSLSSPLIDTERNLGLLLGLHASYLAMSTPLSPVEIECAKWLQSSIFSGGLQTSQIHYSYNEEKDEDHCSSPGGTPASKSRLCSHRRALGDHSQAFLQAIADNNIQDHNVKDFLCQIERYCRQCHLTTPIMFPPEHPVEEVGRLLLCCLLKHEDLGHVALSLVHAGALGIEQVKHRTLPKSVVDVCRVVYQAKCSLIKTHQEQGRSYKEVCAPVIERLRFLFNELRPAVCNDLSIMSKFKLLSSLPRWRRIAQKIIRERRKKRVPKKPESTDDEEKIGNEESDLEEACILPHSPINVDKRPIAIKSPKDKWQPLLSTVTGVHKYKWLKQNVQGLYPQSPLLSTIAEFALKEEPVDVEKMRKCLLKQLERAEVRLEGIDTILKLASKNFLLPSVQYAMFCGWQRLIPEGIDIGEPLTDCLKDVDLIPPFNRMLLEVTFGKLYAWAVQNIRNVLMDASAKFKELGIQPVPLQTITNENPSGPSLGTIPQARFLLVMLSMLTLQHGANNLDLLLNSGMLALTQTALRLIGPSCDNVEEDMNASAQGASATVLEETRKETAPVQLPVSGPELAAMMKIGTRVMRGVDWKWGDQDGPPPGLGRVIGELGEDGWIRVQWDTGSTNSYRMGKEGKYDLKLAELPAAAQPSAEDSDTEDDSEAEQTERNIHPTAMMFTSTINLLQTLCLSAGVHAEIMQSEATKTLCGLLRMLVESGTTDKTSSPNRLVYREQHRSWCTLGFVRSIALTPQVCGALSSPQWITLLMKVVEGHAPFTATSLQRQILAVHLLQAVLPSWDKTERARDMKCLVEKLFDFLGSLLTTCSSDVPLLRESTLRRRRVRPQASLTATHSSTLAEEVVALLRTLHSLTQWNGLINKYINSQLRSITHSFVGRPSEGAQLEDYFPDSENPEVGGLMAVLAVIGGIDGRLRLGGQVMHDEFGEGTVTRITPKGKITVQFSDMRTCRVCPLNQLKPLPAVAFNVNNLPFTEPMLSVWAQLVNLAGSKLEKHKIKKSTKQAFAGQVDLDLLRCQQLKLYILKAGRALLSHQDKLRQILSQPAVQETGTVHTDDGAVVSPDLGDMSPEGPQPPMILLQQLLASATQPSPVKAIFDKQELEAAALAVCQCLAVESTHPSSPGFEDCSSSEATTPVAVQHIRPARVKRRKQSPVPALPIVVQLMEMGFSRRNIEFALKSLTGASGNASSLPGVEALVGWLLDHSDIQVTELSDADTVSDEYSDEEVVEDVDDAAYSMSTGAVVTESQTYKKRADFLSNDDYAVYVRENIQVGMMVRCCRAYEEVCEGDVGKVIKLDRDGLHDLNVQCDWQQKGGTYWVRYIHVELIGYPPPSSSSHIKIGDKVRVKASVTTPKYKWGSVTHQSVGVVKAFSANGKDIIVDFPQQSHWTGLLSEMELVPSIHPGVTCDGCQMFPINGSRFKCRNCDDFDFCETCFKTKKHNTRHTFGRINEPGQSAVFCGRSGKQLKRCHSSQPGMLLDSWSRMVKSLNVSSSVNQASRLIDGSEPCWQSSGSQGKHWIRLEIFPDVLVHRLKMIVDPADSSYMPSLVVVSGGNSLNNLIELKTININPSDTTVPLLNDCTEYHRYIEIAIKQCRSSGIDCKIHGLILLGRIRAEEEDLAAVPFLASDNEEEEDEKGNSGSLIRKKAAGLESAATIRTKVFVWGLNDKDQLGGLKGSKIKVPSFSETLSALNVVQVAGGSKSLFAVTVEGKVYACGEATNGRLGLGISSGTVPIPRQITALSSYVVKKVAVHSGGRHATALTVDGKVFSWGEGDDGKLGHFSRMNCDKPRLIEALKTKRIRDIACGSSHSAALTSSGELYTWGLGEYGRLGHGDNTTQLKPKMVKVLLGHRVIQVACGSRDAQTLALTDEGLVFSWGDGDFGKLGRGGSEGCNIPQNIERLNGQGVCQIECGAQFSLALTKSGVVWTWGKGDYFRLGHGSDVHVRKPQVVEGLRGKKIVHVAVGALHCLAVTDSGQVYAWGDNDHGQQGNGTTTVNRKPTLVQGLEGQKITRVACGSSHSVAWTTVDVATPSVHEPVLFQTARDPLGASYLGVPSDADSSAASNKISGASNSKPNRPSLAKILLSLDGNLAKQQALSHILTALQIMYARDAVVGALMPAAMIAPVECPSFSSAAPSDASAMASPMNGEECMLAVDIEDRLSPNPWQEKREIVSSEDAVTPSAVTPSAPSASARPFIPVTDDLGAASIIAETMTKTKEDVESQNKAAGPEPQALDEFTSLLIADDTRVVVDLLKLSVCSRAGDRGRDVLSAVLSGMGTAYPQVADMLLELCVTELEDVATDSQSGRLSSQPVVVESSHPYTDDTSTSGTVKIPGAEGLRVEFDRQCSTERRHDPLTVMDGVNRIVSVRSGREWSDWSSELRIPGDELKWKFISDGSVNGWGWRFTVYPIMPAAGPKELLSDRCVLSCPSMDLVTCLLDFRLNLASNRSIVPRLAASLAACAQLSALAASHRMWALQRLRKLLTTEFGQSININRLLGENDGETRALSFTGSALAALVKGLPEALQRQFEYEDPIVRGGKQLLHSPFFKVLVALACDLELDTLPCCAETHKWAWFRRYCMASRVAVALDKRTPLPRLFLDEVAKKIRELMADSENMDVLHESHDIFKREQDEQLVQWMNRRPDDWTLSAGGSGTIYGWGHNHRGQLGGIEGAKVKVPTPCEALATLRPVQLIGGEQTLFAVTADGKLYATGYGAGGRLGIGGTESVSTPTLLESIQHVFIKKVAVNSGGKHCLALSSEGEVYSWGEAEDGKLGHGNRSPCDRPRVIESLRGIEVVDVAAGGAHSACVTAAGDLYTWGKGRYGRLGHSDSEDQLKPKLVEALQGHRVVDIACGSGDAQTLCLTDDDTVWSWGDGDYGKLGRGGSDGCKVPMKIDSLTGLGVVKVECGSQFSVALTKSGAVYTWGKGDYHRLGHGSDDHVRRPRQVQGLQGKKVIAIATGSLHCVCCTEDGEVYTWGDNDEGQLGDGTTNAIQRPRLVAALQGKKVNRVACGSAHTLAWSTSKPASAGKLPAQVPMEYNHLQEIPIIALRNRLLLLHHLSELFCPCIPMFDLEGSLDETGLGPSVGFDTLRGILISQGKEAAFRKVVQATMVRDRQHGPVVELNRIQVKRSRSKGGLAGPDGTKSVFGQMCAKMSSFGPDSLLLPHRVWKVKFVGESVDDCGGGYSESIAEICEELQNGLTPLLIVTPNGRDESGANRDCYLLSPAARAPVHSSMFRFLGVLLGIAIRTGSPLSLNLAEPVWKQLAGMSLTIADLSEVDKDFIPGLMYIRDNEATSEEFEAMSLPFTVPSASGQDIQLSSKHTHITLDNRAEYVRLAINYRLHEFDEQVAAVREGMARVVPVPLLSLFTGYELETMVCGSPDIPLHLLKSVATYKGIEPSASLIQWFWEVMESFSNTERSLFLRFVWGRTRLPRTIADFRGRDFVIQVLDKYNPPDHFLPESYTCFFLLKLPRYSCKQVLEEKLKYAIHFCKSIDTDDYARIALTGEPAADDSSDDSDNEDVDSFASDSTQDYLTGH

SEQ ID NO: 123 - protein, 3703 aa, Homo sapiens
  REGION 1..3703: note = dystrophin (DMD) isoform X1
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGHNVGSLFHMADDLGRAMESLVSVMTDEEGAE

SEQ ID NO: 124 - protein, 2471 aa, Homo sapiens
  REGION 1..2471: note = NOTCH 2 (NOTC2)
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCNQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 125 - protein, 2414 aa, synthetic construct
  REGION 1..2414: note = Synthetic construct
  REGION 1..2414: note = p300 (aa)
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 126 - protein, 1052 aa, synthetic construct
  REGION 1..1052: note = Synthetic construct
  REGION 1..1052: note = WT Sa Cas9 (aa)
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 127 - protein, 1367 aa, synthetic construct
  REGION 1..1367: note = Synthetic construct
  REGION 1..1367: note = WT Sp Cas9 (aa)
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 128 - DNA, 66 bp, synthetic construct
  misc_feature 1..66: note = Synthetic construct
  misc_feature 1..66: note = FLAG epitope tag (nt)
  source 1..66: mol_type = other DNA
gactacaaagaccatgacggtgattataaagatcatgacatcgattacaaggatgacgatgacaag

SEQ ID NO: 129 - protein, 22 aa, synthetic construct
  REGION 1..22: note = Synthetic construct
  REGION 1..22: note = FLAG epitope tag (aa)
DYKDHDGDYKDHDIDYKDDDDK

SEQ ID NO: 130 - DNA, 57 bp, synthetic construct
  misc_feature 1..57: note = Synthetic construct
  misc_feature 1..57: note = P2A sequence (nt)
  source 1..57: mol_type = other DNA
gcaacaaacttctctctgctgaaacaagccggagatgtcgaagagaatcctggaccg

SEQ ID NO: 131 - protein, 19 aa, synthetic construct
  REGION 1..19: note = Synthetic construct
  REGION 1..19: note = P2A sequence (aa)
ATNFSLLKQAGDVEENPGP

SEQ ID NO: 132 - DNA, 600 bp, synthetic construct
  misc_feature 1..600: note = Synthetic construct
  misc_feature 1..600: note = Puromycin resistance gene (nt)
  source 1..600: mol_type = other DNA
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtgtcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 133 - protein, 199 aa, synthetic construct
  REGION 1..199: note = Synthetic construct
  REGION 1..199: note = Puromycin resistance gene (aa)
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 134 - protein, 124 aa, synthetic construct
  REGION 1..124: note = Synthetic construct
  REGION 1..124: note = p65AD (aa)
PTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 135 - protein, 190 aa, synthetic construct
RDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 136 - protein, 124 aa, synthetic construct
  REGION 1..124: note = Synthetic construct
  REGION 1..124: note = HSF1 domain (aa)
GFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 137 - protein, 522 aa, synthetic construct
  REGION 1..522: note = Synthetic construct
  REGION 1..522: note = VPR (aa)
DALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 138 - protein, 378 aa, synthetic construct
  REGION 1..378: note = Synthetic construct
  REGION 1..378: note = VPH (aa)
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 139 - protein, 718 aa, synthetic construct
  REGION 1..718: note = Synthetic construct
  REGION 1..718: note = TET1 catalytic domain (aa)
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 140 - protein, 852 aa, synthetic construct
  REGION 1..852: note = Synthetic construct
  REGION 1..852: note = LSD1 (aa)
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQAVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 141-151 - skipped (11 entries)

SEQ ID NO: 152 - protein, 22 aa, synthetic construct
  REGION 1..22: note = Synthetic construct
  REGION 1..22: note = SunTag GCN4 peptide (aa)
LLPKNYHLENEVARLKKLVGER

SEQ ID NO: 153 - protein, 5 aa, synthetic construct
  REGION 1..5: note = Synthetic construct
  REGION 1..5: note = linker(aa)
GGSGG

SEQ ID NO: 154 - protein, 5 aa, synthetic construct
  REGION 1..5: note = Synthetic construct
  REGION 1..5: note = linker
GGGGG

SEQ ID NO: 155 - protein, 5 aa, synthetic construct
  REGION 1..5: note = Synthetic construct
  REGION 1..5: note = linker
GGAGG

SEQ ID NO: 156 - protein, 7 aa, synthetic construct
  REGION 1..7: note = Synthetic construct
  REGION 1..7: note = linker
GGGGSSS

SEQ ID NO: 157 - protein, 7 aa, synthetic construct
  REGION 1..7: note = Synthetic construct
  REGION 1..7: note = linker
GGGGAAA

SEQ ID NO: 158 - protein, 5 aa, synthetic construct
  REGION 1..5: note = Synthetic construct
  REGION 1..5: note = (GGGGS)n linker (aa), n is 1 to 10
GGGGS

SEQ ID NO: 159 - protein, 7 aa, synthetic construct
  REGION 1..7: note = Synthetic construct
  REGION 1..7: note = SV40 virus large T-antigen NLS
PKKKRKV

SEQ ID NO: 160 - protein, 9 aa, synthetic construct
  REGION 1..9: note = Synthetic construct
  REGION 1..9: note = c-myc NLS (aa)
PAAKRVKLD

SEQ ID NO: 161 - protein, 11 aa, synthetic construct
  REGION 1..11: note = Synthetic construct
  REGION 1..11: note = c-myc NLS (aa)
RQRRNELKRSP

SEQ ID NO: 162 - protein, 38 aa, synthetic construct
  REGION 1..38: note = Synthetic construct
  REGION 1..38: note = hRNPA1 M9 NLS (aa)
NQSSNFGPMKGGNFGGRSSGPYGGGGQYFAKPRNQGGY

SEQ ID NO: 163 - protein, 42 aa, synthetic construct
  REGION 1..42: note = Synthetic construct
  REGION 1..42: note = importin-alpha IBB domain NLS (aa)
RMRIZFKNKGKDTAELRRRRVEVSVELRKAKKDEQILKRRNV

SEQ ID NO: 164 - protein, 8 aa, synthetic construct
  REGION 1..8: note = Synthetic construct
  REGION 1..8: note = myoma T protein NLS (aa)
VSRKRPRP

SEQ ID NO: 165 - protein, 8 aa, synthetic construct
  REGION 1..8: note = Synthetic construct
  REGION 1..8: note = myoma T protein NLS (aa)
PPKKARED

SEQ ID NO: 166 - protein, 8 aa, synthetic construct
  REGION 1..8: note = Synthetic construct
  REGION 1..8: note = human p53 NLS (aa)
PQPKKKPL

SEQ ID NO: 167 - protein, 12 aa, synthetic construct
  REGION 1..12: note = Synthetic construct
  REGION 1..12: note = mouse c-abl IV NLS (aa)
SALIKKKKKMAP

SEQ ID NO: 168 - protein, 5 aa, synthetic construct
  REGION 1..5: note = Synthetic construct
  REGION 1..5: note = influenza virus NS1 NLS (aa)
DRLRR

SEQ ID NO: 169 - protein, 7 aa, synthetic construct
  REGION 1..7: note = Synthetic construct
  REGION 1..7: note = influenza virus NS1 NLS (aa)
PKQKKRK

SEQ ID NO: 170 - protein, 10 aa, synthetic construct
  REGION 1..10: note = Synthetic construct
  REGION 1..10: note = Hepatitis virus delta antigen NLS (aa)
RKLKKKIKKL

SEQ ID NO: 171 - protein, 10 aa, synthetic construct
  REGION 1..10: note = Synthetic construct
  REGION 1..10: note = mouse Mx1 protein MNLS (aa)
REKKKFLKRR

SEQ ID NO: 172 - protein, 20 aa, synthetic construct
  REGION 1..20: note = Synthetic construct
  REGION 1..20: note = human poly(ADP-ribose) polymerase NLS (aa)
KRKGDEVDGVDEVAKKKSKK

SEQ ID NO: 173 - protein, 17 aa, synthetic construct
  REGION 1..17: note = Synthetic construct
  REGION 1..17: note = steroid hormone receptors (human) glucocorticoidNLS (aa)
RKCLQAGMNLEARKTKK

SEQ ID NO: 174 - protein, 16 aa, synthetic construct
  REGION 1..16: note = Synthetic construct
  REGION 1..16: note = linker
SGSETPGTSESATPES

SEQ ID NO: 175 - DNA, 1716 bp, synthetic construct
  misc_feature 1..1716: note = Synthetic construct
  misc_feature 1..1716: note = N term dSpCas9 N terminal fragement (nt)
  source 1..1716: mol_type = other DNA
gacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaa

SEQ ID NO: 176 - protein, 572 aa, synthetic construct
  REGION 1..572: note = Synthetic construct
  REGION 1..572: note = N term dSpCas9 N terminal fragement
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIE

SEQ ID NO: 177 - DNA, 255 bp, synthetic construct
  misc_feature 1..255: note = Synthetic construct
  misc_feature 1..255: note = N term Npu Intein (nt)
  source 1..255: mol_type = other DNA
tgcctgtcctacgagacagagatcctgacagtggagtatggcctgctgccaatcggcaagatcgtggagaagaggatcgagtgtaccgtgtactctgtggataacaatggcaacatctatacacagcccgtggcacagtggcacgataggggagagcaggaggtgttcgagtattgcctggaggacggcagcctgatcagggcaaccaaggaccacaagttcatgacagtggatggccagatgctgcccatcgac

SEQ ID NO: 178 - protein, 85 aa, synthetic construct
  REGION 1..85: note = Synthetic construct
  REGION 1..85: note = N term Npu Intein (aa)
CLSYETEILTVEYGLLPIGKIVEKRIECTVYSVDNNGNIYTQPVAQWHDRGEQEVFEYCLEDGSLIRATKDHKFMTVDGQMLPID

SEQ ID NO: 179 - DNA, 2598 bp, synthetic construct
  misc_feature 1..2598: note = Synthetic construct
  misc_feature 1..2598: note = dSpCas9-573-C Intein (nt)
  source 1..2598: mol_type = other DNA
atgaaacggacagccgacggaagcgagttcgagtcaccaaagaagaagcggaaagtcatcaagattgctacacggaaatacctgggaaagcagaacgtgtacgacatcggcgtggagcgggatcacaacttcgccctgaagaatggctttatcgccagcaattgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacaaaaggccggcggccacgaaaaaggccggccaggcaaaaaagaaaaagtga

SEQ ID NO: 180 - protein, 865 aa, synthetic construct
  REGION 1..865: note = Synthetic construct
  REGION 1..865: note = dSpCas9-573-C Intein (aa)
MKRTADGSEFESPKKKRKVIKIATRKYLGKQNVYDIGVERDHNFALKNGFIASNCFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 181 - DNA, 105 bp, synthetic construct
  misc_feature 1..105: note = Synthetic construct
  misc_feature 1..105: note = C term Npu Intein (nt)
  source 1..105: mol_type = other DNA
atcaagattgctacacggaaatacctgggaaagcagaacgtgtacgacatcggcgtggagcgggatcacaacttcgccctgaagaatggctttatcgccagcaat

SEQ ID NO: 182 - protein, 35 aa, synthetic construct
  REGION 1..35: note = Synthetic construct
  REGION 1..35: note = C term Npu Intein (aa)
IKIATRKYLGKQNVYDIGVERDHNFALKNGFIASN

SEQ ID NO: 183 - DNA, 2385 bp, synthetic construct
  misc_feature 1..2385: note = Synthetic construct
  misc_feature 1..2385: note = C term dSpCas9 C terminal fragment (nt)
  source 1..2385: mol_type = other DNA
tgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 184 - protein, 795 aa, synthetic construct
  REGION 1..795: note = Synthetic construct
  REGION 1..795: note = C term dSpCas9 C terminal fragment (aa)
CFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 185 - DNA, 240 bp, synthetic construct
  misc_feature 1..240: note = Synthetic construct
  misc_feature 1..240: note = extended linker (nt)
  source 1..240: mol_type = other DNA
ggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgag

SEQ ID NO: 186 - protein, 80 aa, synthetic construct
  REGION 1..80: note = Synthetic construct
  REGION 1..80: note = extended linker (nt)
GGPSSGAPPPSGGSPAGSPTSTEEGTSESATPESGPGTSTEPSEGSAPGSPAGSPTSTEEGTSTEPSEGSAPGTSTEPSE

SEQ ID NO: 187 - DNA, 48 bp, synthetic construct
  misc_feature 1..48: note = Synthetic construct
  misc_feature 1..48: note = linker (nt)
  source 1..48: mol_type = other DNA
aacccaaagaagaagcggaaggtcggtatccacggagtcccagcagcc

SEQ ID NO: 188 - protein, 16 aa, synthetic construct
  REGION 1..16: note = Synthetic construct
  REGION 1..16: note = linker (aa)
NPKKKRKVGIHGVPAA

SEQ ID NO: 189 - protein, 1053 aa, synthetic construct
  REGION 1..1053: note = Synthetic construct
  REGION 1..1053: note = dSaCas9 (aa)
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 190 - protein, 1368 aa, synthetic construct
  REGION 1..1368: note = Synthetic construct
  REGION 1..1368: note = dSpCas9 (aa)
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 191 - protein, 1307 aa, synthetic construct
  REGION 1..1307: note = Synthetic construct
  REGION 1..1307: note = dAsCas12a (dAsCpf1) (aa)
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLANLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 192 - protein, 1306 aa, synthetic construct
  REGION 1..1306: note = Synthetic construct
  REGION 1..1306: note = dAsCas12a (dAsCpf1) (aa)
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLANLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 193 - protein, 1307 aa, synthetic construct
  REGION 1..1307: note = Synthetic construct
  REGION 1..1307: note = WT AsCas12a (AsCpf1) (aa)
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 194 - protein, 1306 aa, synthetic construct
  REGION 1..1306: note = Synthetic construct
  REGION 1..1306: note = WT AsCas12a (AsCpf1) (aa)
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 195 - protein, 776 aa, synthetic construct
  REGION 1..776: note = Synthetic construct
  REGION 1..776: note = dCasPhi-2 (aa)
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIALGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTSGSGPKKKRKVEDPKKKRKV

SEQ ID NO: 196 - protein, 775 aa, synthetic construct
  REGION 1..775: note = Synthetic construct
  REGION 1..775: note = dCasPhi-2 (aa)
PKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIALGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTSGSGPKKKRKVEDPKKKRKV

SEQ ID NO: 197 - protein, 757 aa, synthetic construct
  REGION 1..757: note = Synthetic construct
  REGION 1..757: note = WT CasPhi-2(aa)
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 198 - protein, 756 aa, synthetic construct
  REGION 1..756: note = Synthetic construct
  REGION 1..756: note = WT CasPhi-2 (aa)
PKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 199 - protein, 529 aa, synthetic construct
  REGION 1..529: note = Synthetic construct
  REGION 1..529: note = WT Un1Cas12f1 (aa)
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 200 - protein, 528 aa, synthetic construct
  REGION 1..528: note = Synthetic construct
  REGION 1..528: note = WT Un1Cas12f1 (aa)
AKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 201 - protein, 529 aa, synthetic construct
  REGION 1..529: note = Synthetic construct
  REGION 1..529: note = dUn1Cas12f1 (aa)
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 202 - protein, 528 aa, synthetic construct
  REGION 1..528: note = Synthetic construct
  REGION 1..528: note = dUn1Cas12f1 (aa)
AKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKEEP

SEQ ID NO: 203 - protein, 984 aa, synthetic construct
  REGION 1..984: note = Synthetic construct
  REGION 1..984: note = dCjCas9 (aa)
MARILAFAIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDAIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 204 - protein, 983 aa, synthetic construct
  REGION 1..983: note = Synthetic construct
  REGION 1..983: note = dCjCas9 (aa)
ARILAFAIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDAIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 205 - protein, 836 aa, synthetic construct
  REGION 1..836: note = Synthetic construct
  REGION 1..836: note = WT CjCas9 (aa)
MSSGSGHMARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELGGGSGGYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 206 - protein, 835 aa, synthetic construct
  REGION 1..835: note = Synthetic construct
  REGION 1..835: note = WT CjCas9 (aa)
SSGSGHMARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELGGGSGGYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 207 - protein, 663 aa, synthetic construct
  REGION 1..663: note = Synthetic construct
  REGION 1..663: note = WT Cas12k (aa)
MSSTSGKNPTNPIVRTISCNLSAKEDVLRKVWEEMSQKNTPLIVQLLKSVSEQPEFEANKENGTITKKEITELRRDITEDSDLKKQSGRLRSSADSLVTEVYSSWLRLYQVRKNKKEGKEYFLNNILKSDVELVEQSNCDLQTIRCKAKEILSQVEEFIEQVNNKPKINKTTSAKKKINKSNKNNKAIEEILNRFFIGNIDKTLTNTLYEIHRKSPDILTQCAVAYLIKNDNKVSEAEENLTQLNKRSSAKEIEIKRLETQIQNTRLPNGRDITGEKYSQAFEKLVNQVPQDNEEFAEWIAILLKKVSSLPYPILYSSGDLSWYKDEKGNIFVYFNGWAEYHFQICCDKRQLRFFERFLKYYKALKASEKGEEKLSGSLVTLRSAHLLWRQGKGKGEPWKVNKLALHCTYDARLWTAEGTEEVRQEKTDKAQAEVNQAESNENIDSKQQKKLTKNKSSLSRLKNSFARPSKPLYRGQSNIIVGISFHPVELATLVVVDINTKEILICKTVKELLGDAFPLLSRRRRQQVHFRKEREKAQKKDSPCDLGESKLGEYVDRLLAKRIVEVAKEYQASCIVLPGLKGIREIRTSVIQAKAETKFPGDINAQELYVKEYNRQIHNWSYSRLQESIKSRAAELKISIKFGKQPSHSTLQEQAINLALSA

SEQ ID NO: 208 - protein, 662 aa, synthetic construct
  REGION 1..662: note = Synthetic construct
  REGION 1..662: note = WT Cas12k (aa)
SSTSGKNPTNPIVRTISCNLSAKEDVLRKVWEEMSQKNTPLIVQLLKSVSEQPEFEANKENGTITKKEITELRRDITEDSDLKKQSGRLRSSADSLVTEVYSSWLRLYQVRKNKKEGKEYFLNNILKSDVELVEQSNCDLQTIRCKAKEILSQVEEFIEQVNNKPKINKTTSAKKKINKSNKNNKAIEEILNRFFIGNIDKTLTNTLYEIHRKSPDILTQCAVAYLIKNDNKVSEAEENLTQLNKRSSAKEIEIKRLETQIQNTRLPNGRDITGEKYSQAFEKLVNQVPQDNEEFAEWIAILLKKVSSLPYPILYSSGDLSWYKDEKGNIFVYFNGWAEYHFQICCDKRQLRFFERFLKYYKALKASEKGEEKLSGSLVTLRSAHLLWRQGKGKGEPWKVNKLALHCTYDARLWTAEGTEEVRQEKTDKAQAEVNQAESNENIDSKQQKKLTKNKSSLSRLKNSFARPSKPLYRGQSNIIVGISFHPVELATLVVVDINTKEILICKTVKELLGDAFPLLSRRRRQQVHFRKEREKAQKKDSPCDLGESKLGEYVDRLLAKRIVEVAKEYQASCIVLPGLKGIREIRTSVIQAKAETKFPGDINAQELYVKEYNRQIHNWSYSRLQESIKSRAAELKISIKFGKQPSHSTLQEQAINLALSA

SEQ ID NO: 209 - protein, 558 aa, synthetic construct
  REGION 1..558: note = Synthetic construct
  REGION 1..558: note = CasMini
MGPKKKRKVGSGSAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERPAKRPAATKKAGQAKKKK

SEQ ID NO: 210 - protein, 557 aa, synthetic construct
  REGION 1..557: note = Synthetic construct
  REGION 1..557: note = CasMini
GPKKKRKVGSGSAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERPAKRPAATKKAGQAKKKK

SEQ ID NO: 211 - RNA, 29 nt, synthetic construct
  misc_feature 1..29: note = Synthetic construct
  misc_feature 1..29: note = dAsCas12a gRNA scaffold
  source 1..29: mol_type = other RNA
gaaacaccgtaatttctactcttgtagat

SEQ ID NO: 212 - RNA, 25 nt, synthetic construct
  misc_feature 1..25: note = Synthetic construct
  misc_feature 1..25: note = dCasPhi-2 gRNA scaffold
  source 1..25: mol_type = other RNA
caacgattgcccctcacgaggggac

SEQ ID NO: 213 - RNA, 90 nt, synthetic construct
  misc_feature 1..90: note = Synthetic construct
  misc_feature 1..90: note = Un1Cas12f1 gRNA scaffold
  source 1..90: mol_type = other RNA
accgcttcacttagagtgaaggtgggctgcttgcatcagcctaatgtcgagaagtgctttcttcggaaagtaaccctcgaaacaaagaaa

SEQ ID NO: 214-215 - skipped (2 entries)

SEQ ID NO: 216 - RNA, 73 nt, synthetic construct
  misc_feature 1..73: note = Synthetic construct
  misc_feature 1..73: note = CjCas9 gRNA scaffold
  source 1..73: mol_type = other RNA
gcggttttaggggattgtaaccccgcagagtcccgcaaactctttattttagtcccttttcagggactaaaac

SEQ ID NO: 217 - RNA, 235 nt, synthetic construct
  misc_feature 1..235: note = Synthetic construct
  misc_feature 1..235: note = Cas12k gRNA scaffold
  source 1..235: mol_type = other RNA
atattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctccaaaaggtgggttgaaag

SEQ ID NO: 218 - RNA, 160 nt, synthetic construct
  misc_feature 1..160: note = Synthetic construct
  misc_feature 1..160: note = CasMini gRNA scaffold
  source 1..160: mol_type = other RNA
gggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaac

SEQ ID NO: 219 - protein, 5 aa, synthetic construct
  REGION 1..5: note = Synthetic construct
  REGION 1..5: note = linker (aa)
GSGSG

SEQ ID NO: 220 - protein, 757 aa, synthetic construct
  REGION 1..757: note = Synthetic construct
  REGION 1..757: note = dCasPhi-2
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIALGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 221 - protein, 756 aa, synthetic construct
  REGION 1..756: note = Synthetic construct
  REGION 1..756: note = dCasPhi-2
PKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIALGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 222 - skipped

SEQ ID NO: 223 - protein, 528 aa, synthetic construct
  REGION 1..528: note = Synthetic construct
  REGION 1..528: note = CasMini Cas12f/D326A/D510A/D143R/T147R/K330R/E528R(aa)
AKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSRVCYRRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKICEKSAWMLNLSIDVPKIDKGVDPSIIGGIAVGVRSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENAAYNAALNISNPKLKSTKERP

SEQ ID NO: 224-228 - skipped (5 entries)

SEQ ID NO: 229 - DNA, 23 bp, synthetic construct
  misc_feature 1..23: note = Synthetic construct
  misc_feature 1..23: note = Cas12a gRNA 1 spacer
  source 1..23: mol_type = other DNA
tggaagtaaggcaaaaagattgc

SEQ ID NO: 230 - DNA, 23 bp, synthetic construct
  misc_feature 1..23: note = Synthetic construct
  misc_feature 1..23: note = Cas12a gRNA 2 spacer
  source 1..23: mol_type = other DNA
aggcaaaaagattgctttgaata

SEQ ID NO: 231 - DNA, 23 bp, synthetic construct
  misc_feature 1..23: note = Synthetic construct
  misc_feature 1..23: note = Cas12a gRNA 3 spacer
  source 1..23: mol_type = other DNA
agtggttctcaaccaggggcaat

SEQ ID NO: 232 - DNA, 23 bp, synthetic construct
  misc_feature 1..23: note = Synthetic construct
  misc_feature 1..23: note = Cas12a gRNA 4 spacer
  source 1..23: mol_type = other DNA
caattttactccttaggggacct

SEQ ID NO: 233 - DNA, 23 bp, synthetic construct
  misc_feature 1..23: note = Synthetic construct
  misc_feature 1..23: note = Cas12a gRNA 5 spacer
  source 1..23: mol_type = other DNA
ttttcacaatgtctggagacatt

SEQ ID NO: 234 - DNA, 23 bp, synthetic construct
  misc_feature 1..23: note = Synthetic construct
  misc_feature 1..23: note = Cas12a gRNA 6 spacer
  source 1..23: mol_type = other DNA
tgggaaatgcatcattaggtgat

SEQ ID NO: 235 - DNA, 23 bp, synthetic construct
  misc_feature 1..23: note = Synthetic construct
  misc_feature 1..23: note = Cas12a gRNA 7 spacer
  source 1..23: mol_type = other DNA
aattgtactacagtagttaagta

SEQ ID NO: 236 - DNA, 23 bp, synthetic construct
  misc_feature 1..23: note = Synthetic construct
  misc_feature 1..23: note = Cas12a gRNA 8 spacer
  source 1..23: mol_type = other DNA
agtatggtaacatgctgtacagg

SEQ ID NO: 237 - DNA, 23 bp, synthetic construct
  misc_feature 1..23: note = Synthetic construct
  misc_feature 1..23: note = Cas12a gRNA 9 spacer
  source 1..23: mol_type = other DNA
tatagtaggctagaccatctagc

SEQ ID NO: 238 - DNA, 23 bp, synthetic construct
  misc_feature 1..23: note = Synthetic construct
  misc_feature 1..23: note = Cas12a gRNA 10 spacer
  source 1..23: mol_type = other DNA
tcacaaaatcacctaatgatgca

SEQ ID NO: 239 - DNA, 23 bp, synthetic construct
  misc_feature 1..23: note = Synthetic construct
  misc_feature 1..23: note = Cas12a gRNA 11 spacer
  source 1..23: mol_type = other DNA
ttgtcccatgcattgtaaggtgg

SEQ ID NO: 240 - DNA, 23 bp, synthetic construct
  misc_feature 1..23: note = Synthetic construct
  misc_feature 1..23: note = Cas12a gRNA 12 spacer
  source 1..23: mol_type = other DNA
gagaaccactattctggtctaac

SEQ ID NO: 241 - DNA, 23 bp, synthetic construct
  misc_feature 1..23: note = Synthetic construct
  misc_feature 1..23: note = Cas12a gRNA 13 spacer
  source 1..23: mol_type = other DNA
aattaaatattcaaagcaatctt

SEQ ID NO: 242 - DNA, 23 bp, synthetic construct
  misc_feature 1..23: note = Synthetic construct
  misc_feature 1..23: note = Cas12a gRNA 14 spacer
  source 1..23: mol_type = other DNA
atctttttgccttacttccaagt

SEQ ID NO: 243 - DNA, 23 bp, synthetic construct
  misc_feature 1..23: note = Synthetic construct
  misc_feature 1..23: note = Cas12a gRNA 15 spacer
  source 1..23: mol_type = other DNA
cttacttccaagttttagataat

SEQ ID NO: 244 - DNA, 23 bp, synthetic construct
  misc_feature 1..23: note = Synthetic construct
  misc_feature 1..23: note = Cas12a gRNA 16 spacer
  source 1..23: mol_type = other DNA
gctcacgcctgtcgtctcagcac

SEQ ID NO: 245 - DNA, 23 bp, synthetic construct
  misc_feature 1..23: note = Synthetic construct
  misc_feature 1..23: note = Cas12a gRNA 17 spacer
  source 1..23: mol_type = other DNA
gtgaggcccaggcgggcagatcg

SEQ ID NO: 246 - DNA, 23 bp, synthetic construct
  misc_feature 1..23: note = Synthetic construct
  misc_feature 1..23: note = Cas12a gRNA 18 spacer
  source 1..23: mol_type = other DNA
cagcagctcccaagttcctcctg

SEQ ID NO: 247 - DNA, 23 bp, synthetic construct
  misc_feature 1..23: note = Synthetic construct
  misc_feature 1..23: note = Cas12a gRNA 19 spacer
  source 1..23: mol_type = other DNA
cccaagttcctcctgtttagaat

SEQ ID NO: 248 - DNA, 23 bp, synthetic construct
  misc_feature 1..23: note = Synthetic construct
  misc_feature 1..23: note = Cas12a gRNA 20 spacer
  source 1..23: mol_type = other DNA
taatattttcaaggctggatttt

SEQ ID NO: 249 - DNA, 23 bp, synthetic construct
  misc_feature 1..23: note = Synthetic construct
  misc_feature 1..23: note = Cas12a gRNA 21 spacer
  source 1..23: mol_type = other DNA
ctggatttttttgaacgaaatgc

SEQ ID NO: 250 - DNA, 23 bp, synthetic construct
  misc_feature 1..23: note = Synthetic construct
  misc_feature 1..23: note = Cas12a gRNA 22 spacer
  source 1..23: mol_type = other DNA
tagctattctgcagctctgaaag

SEQ ID NO: 251 - DNA, 23 bp, synthetic construct
  misc_feature 1..23: note = Synthetic construct
  misc_feature 1..23: note = Cas12a gRNA 23 spacer
  source 1..23: mol_type = other DNA
tttcacctcgttccaggaaagca

SEQ ID NO: 252 - DNA, 23 bp, synthetic construct
  misc_feature 1..23: note = Synthetic construct
  misc_feature 1..23: note = Cas12a gRNA 24 spacer
  source 1..23: mol_type = other DNA
tcactgcaagctctgcctcccgg

SEQ ID NO: 253 - DNA, 23 bp, synthetic construct
  misc_feature 1..23: note = Synthetic construct
  misc_feature 1..23: note = Cas12a gRNA 25 spacer
  source 1..23: mol_type = other DNA
ccgccaccatgcctggctaattt

SEQ ID NO: 254 - DNA, 23 bp, synthetic construct
  misc_feature 1..23: note = Synthetic construct
  misc_feature 1..23: note = Cas12a gRNA 26 spacer
  source 1..23: mol_type = other DNA
acacccagctagtttttgtattt

SEQ ID NO: 255 - DNA, 23 bp, synthetic construct
  misc_feature 1..23: note = Synthetic construct
  misc_feature 1..23: note = Cas12a gRNA 27 spacer
  source 1..23: mol_type = other DNA
ttgtattttttgtagaaaggggg

SEQ ID NO: 256 - DNA, 20 bp, synthetic construct
  misc_feature 1..20: note = Synthetic construct
  misc_feature 1..20: note = gRNA V spacer
  source 1..20: mol_type = other DNA
aagtaaggcaaaaagattgc

SEQ ID NO: 257 - DNA, 20 bp, synthetic construct
  misc_feature 1..20: note = Synthetic construct
  misc_feature 1..20: note = gRNA W spacer
  source 1..20: mol_type = other DNA
caattttactccttagggga

SEQ ID NO: 258 - DNA, 20 bp, synthetic construct
  misc_feature 1..20: note = Synthetic construct
  misc_feature 1..20: note = gRNA X spacer
  source 1..20: mol_type = other DNA
gagagcagatactactgcca

SEQ ID NO: 259 - DNA, 20 bp, synthetic construct
  misc_feature 1..20: note = Synthetic construct
  misc_feature 1..20: note = gRNA Y spacer
  source 1..20: mol_type = other DNA
tggagaccaagaatgctgct

SEQ ID NO: 260 - DNA, 20 bp, synthetic construct
  misc_feature 1..20: note = Synthetic construct
  misc_feature 1..20: note = gRNA Z spacer
  source 1..20: mol_type = other DNA
tgattttgtgatcttgcgaa

SEQ ID NO: 261 - DNA, 20 bp, synthetic construct
  misc_feature 1..20: note = Synthetic construct
  misc_feature 1..20: note = gRNA AA spacer
  source 1..20: mol_type = other DNA
taacatgctgtacaggttta

SEQ ID NO: 262 - DNA, 20 bp, synthetic construct
  misc_feature 1..20: note = Synthetic construct
  misc_feature 1..20: note = gRNA AB spacer
  source 1..20: mol_type = other DNA
ctagaccatctagctttgtg

SEQ ID NO: 263 - DNA, 20 bp, synthetic construct
  misc_feature 1..20: note = Synthetic construct
  misc_feature 1..20: note = gRNA AC spacer
  source 1..20: mol_type = other DNA
tctccacccactagatggca

SEQ ID NO: 264 - DNA, 20 bp, synthetic construct
  misc_feature 1..20: note = Synthetic construct
  misc_feature 1..20: note = gRNA AD spacer
  source 1..20: mol_type = other DNA
tcccctaaggagtaaaattg

SEQ ID NO: 265 - DNA, 20 bp, synthetic construct
  misc_feature 1..20: note = Synthetic construct
  misc_feature 1..20: note = gRNA AE spacer
  source 1..20: mol_type = other DNA
tctaactttgaattaaatat

(19) United States
(12) Patent Application Publication
(10) Pub. No.: US 2024/0254483 A1
(43) Pub. Date: Aug. 1, 2024

(54) COMPOSITIONS AND METHODS FOR MODULATING EXPRESSION OF FRATAXIN (FXN)

(71) Applicant: Tune Therapeutics, Inc., Seattle, WA (US)

(72) Inventors: Matthew P. GEMBERLING, Durham, NC (US); Jennifer KWON, Durham, NC (US); Matthew WOLPERT, Durham, NC (US); Charles A. GERSBACH, Durham, NC (US); Kendra CONGDON, Durham, NC (US); Dilara SEN, Durham, NC (US)

(73) Assignee: Tune Therapeutics, Inc., Seattle, WA (US)

(21) Appl. No.: 18/293,325

(22) PCT Filed: Jul. 29, 2022
(86) PCT No.: PCT/US2022/074353

§ 371 Date: Jan. 29, 2024

Related U.S. Application Data
(60) Provisional application No. 63/388,590, filed on Jul. 12, 2022.
(60) Provisional application No. 63/228,015, filed on Jul. 30, 2021.

(51) Int. Cl. C12N 15/113; A61P 25/00; C12N 9/22; C12N 15/90

(52) U.S. Cl. CPC C12N 15/113; A61P 25/00; C12N 9/22; C12N 15/907; C07K 2319/80; C12N 2310/20

(57) ABSTRACT

Provided in some aspects are compositions, such as DNA-targeting systems, fusion proteins, guide RNAs (gRNAs), and pluralities and combinations thereof, that bind to or target a frataxin (FXN) locus. In particular, the present disclosure relates to the modulation of expression of the FXN gene. In some aspects, the present disclosure also relates to poly nucleotides, vectors, cells and pluralities and combinations thereof, that encode or comprise the DNA-targeting systems, fusion proteins, gRNAs or pluralities or combinations thereof, and methods and uses related to the provided compositions, for example, in modulating the expression of FXN, and/or in the treatment or therapy of diseases or disorders that involve the activity, function or expression of FXN, such as Friedreich's Ataxia (FA).

### CROSS-REFERENCE TO RELATED APPLICATIONS

This application claims priority from U.S. provisional application No. 63/228,015, filed Jul. 30, 2021, entitled “COMPOSITIONS AND METHODS FOR MODULATING EXPRESSION OF FRATAXIN (FXN),” and U.S. provisional application No. 63/388,590, filed Jul. 12, 2022, entitled “COMPOSITIONS AND METHODS FOR MODULATING EXPRESSION OF FRATAXIN (FXN),” the contents of which are incorporated by reference in their entireties.

### INCORPORATION BY REFERENCE OF SEQUENCE LISTING

The present application is being filed along with a Sequence Listing in electronic format. The Sequence Listing is provided as a file entitled 224742000140.xml, created Jul. 28, 2022, which is 506 kilobytes in size. The information in the electronic format of the Sequence Listing is incorporated by reference in its entirety.

### FIELD

The present disclosure relates in some aspects to compositions, such as DNA-targeting systems, fusion proteins, guide RNAs (gRNAs), and pluralities and combinations thereof, that bind to or target a frataxin (FXN) locus. In particular, the present disclosure relates to the modulation of expression of the FXN gene. In some aspects, the present disclosure also relates to polynucleotides, vectors, cells and pluralities and combinations thereof, that encode or comprise the DNA-targeting systems, fusion proteins, gRNAs or pluralities or combinations thereof, and methods and uses related to the provided compositions, for example, in modulating the expression of FXN, and/or in the treatment or therapy of diseases or disorders that involve the activity, function or expression of FXN, such as Friedreich's Ataxia (FA).

### BACKGROUND

Genetic development disorders, including FA, are associated with reduced activity, mutation and/or dysregulation of expression of the frataxin (FXN) gene. FA is an autosomal recessive neurodegenerative and cardiac disease, is caused by a trinucleotide repeat expansion mutation in the FXN gene. FA can result in ataxia, areflexia, loss of vibratory sense and proprioception, dysarthria, cardiomyopathy and/or associated arrhythmias, among other symptoms. Existing treatment of such genetic disorders are directed towards symptoms and providing support. Treatments that address the fundamental etiology and disease mechanism are needed. Provided are embodiments that meet such needs.

### SUMMARY

Provided herein DNA-targeting systems that bind to or target a frataxin (FXN) locus. In some aspects, the DNA-targeting systems include fusion proteins. In some aspects, the DNA-targeting systems include guide RNAs (gRNAs). In some aspects, the DNA-targeting systems include fusion proteins and gRNAs. Provided herein are compositions, such as DNA-targeting systems, including fusion proteins, gRNAs, and pluralities and combinations thereof, that bind to or target a FXN locus. Also provided are fusion proteins that bind to or target FXN. Also provided are gRNAs that bind to or target FXN. In some aspects, the provided DNA-targeting systems, including fusion proteins, gRNAs, bind to, target, and/or modulate the expression of FXN. Also provided are compositions, such as polynucleotides, vectors, cells, and pluralities and combinations thereof, that encode or comprise the DNA-targeting systems, fusion proteins, gRNAs or components thereof. Also provided are methods and uses related to any compositions, for example, in modulating the expression of FXN, and/or in the treatment or therapy of diseases or disorders that involve the activity, function or expression of FXN, such as FA.

Provided herein are DNA-targeting systems comprising a DNA-targeting domain that binds to a target site in a regulatory DNA element of a frataxin (FXN) locus. In some of any embodiments, the DNA-targeting system also includes at least one effector domain that increases transcription of the FXN locus. In some of any embodiments, binding of the DNA-targeting domain to the target site does not introduce a genetic disruption or a DNA break at or near the target site.

Provided herein are DNA-targeting systems comprising a DNA-targeting domain that binds to a target site in a regulatory DNA element of a frataxin (FXN) locus; and at least one effector domain that increases transcription of the FXN locus.

Provided herein are DNA-targeting systems comprising a DNA-targeting domain that binds to a target site in an enhancer of a frataxin (FXN) locus.

Provided herein are DNA-targeting systems comprising a DNA-targeting domain that binds to a target site in an enhancer of a frataxin (FXN) locus; and (b) at least one effector domain that increases transcription of the FXN locus.

In some of any embodiments, the DNA-targeting domain comprises a Clustered Regularly Interspaced Short Palindromic Repeats associated (Cas)-guide RNA (gRNA) combination that includes (a) a Cas protein or a variant thereof and (b) at least one gRNA; a zinc finger protein (ZFP); a transcription activator-like effector (TALE); a meganuclease; a homing endonuclease; or a I-SceI enzymes or a variant thereof. In some embodiments, the DNA-targeting domain comprises a catalytically inactive variant of any of the foregoing.

In some of any embodiments, the DNA-targeting domain comprises a Cas-gRNA combination that includes (a) a Cas protein or a variant thereof and (b) at least one gRNA. In some of any embodiments, the variant Cas protein lacks nuclease activity or is a deactivated Cas (dCas) protein.

Also provided herein are DNA-targeting systems comprising a DNA-targeting domain, that binds to a target site in a regulatory DNA element of a frataxin (FXN) locus and comprises a Cas-guide RNA (gRNA) combination comprising: (a) a variant Cas protein that lacks nuclease activity or that is a deactivated Cas (dCas) protein; and (b) at least one gRNA comprising at least one gRNA spacer sequence (i.e. guide sequence) that is capable of hybridizing to the target site or is complementary to the target site.

Also provided herein are DNA-targeting systems comprising a DNA-targeting domain that is a Cas-guide RNA (gRNA) combination comprising: (a) a deactivated Cas (dCas) protein; (b) at least one effector domain that increases transcription of a frataxin (FXN) locus; and (c) at least one gRNA comprising a gRNA spacer sequence that is capable of hybridizing to a target site in a regulatory DNA element of the FXN locus or is complementary to the target site.

In some of any embodiments, the gRNA is capable of complexing with the Cas protein or variant thereof. In some of any embodiments, the gRNA comprises at least one gRNA spacer sequence that is capable of hybridizing to the target site or is complementary to the target site.

Also provided herein are DNA-targeting systems comprising a DNA-targeting domain that is a Cas-guide RNA (gRNA) combination comprising: (a) Staphylococcus aureus dCas9 protein (dSaCas9); (b) at least one effector domain that increases transcription of a frataxin (FXN) locus; and (c) at least one gRNA, comprising a gRNA spacer sequence that is capable of hybridizing to a target site in a regulatory DNA element of the FXN locus or is complementary to the target site.

Also provided herein are DNA-targeting systems comprising a DNA-targeting domain that is a Cas-guide RNA (gRNA) combination comprising: (a) Staphylococcus aureus dCas9 protein (dSaCas9); and (b) at least one gRNA, comprising a gRNA spacer sequence that is capable of hybridizing to a target site in an enhancer of a frataxin (FXN) locus or is complementary to the target site.

Also provided herein are DNA-targeting systems comprising a DNA-targeting domain that is a Cas-guide RNA (gRNA) combination comprising: (a) Staphylococcus aureus dCas9 protein (dSaCas9); (b) at least one effector domain that increases transcription of a frataxin (FXN) locus; and (c) at least one gRNA, comprising a gRNA spacer sequence that is capable of hybridizing to a target site in an enhancer of the FXN locus or is complementary to the target site.

In some of any embodiments, the Cas protein or a variant thereof is a Cas9 protein or a variant thereof. In some of any embodiments, the variant Cas protein is a variant Cas9 protein that lacks nuclease activity or that is a deactivated Cas9 (dCas9) protein. In some of any embodiments, the Cas9 protein or a variant thereof is a Staphylococcus aureus Cas9 (SaCas9) protein or a variant thereof. In some of any embodiments, the variant Cas9 is a Staphylococcus aureus dCas9 protein (dSaCas9) that comprises at least one amino acid mutation selected from D10A and N580A, with reference to numbering of positions of SEQ ID NO:73. In some of any embodiments, the variant Cas9 protein comprises the sequence set forth in SEQ ID NO:72, or an amino acid sequence that has at least 90%, 91%, 92%, 93%, 94%, 95%, 96%, 97%, 98%, or 99% sequence identity thereto.

In some of any embodiments, the Cas9 protein or variant thereof is a Streptococcus pyogenes Cas9 (SpCas9) protein or a variant thereof. In some of any embodiments, the variant Cas9 is a Streptococcus pyogenes dCas9 (dSpCas9) protein that comprises at least one amino acid mutation selected from D10A and H840A, with reference to numbering of positions of SEQ ID NO:79. In some of any embodiments, the variant Cas9 protein comprises the sequence set forth in SEQ ID NO:78, or an amino acid sequence that has at least 90%, 91%, 92%, 93%, 94%, 95%, 96%, 97%, 98%, or 99% sequence identity thereto.

In some of any embodiments, the Cas protein or a variant thereof is a split variant Cas protein, wherein the split variant Cas protein comprises a first polypeptide comprising an N-terminal fragment of the variant Cas protein and an N-terminal Intein, and a second polypeptide comprising a C-terminal fragment of the variant Cas protein and a C-terminal Intein. In some of any embodiments, when the first polypeptide and the second polypeptide of the split variant Cas protein are present in proximity or present in the same cell, the N-terminal Intein and C-terminal Intein self-excise and ligate the N-terminal fragment and the C-terminal fragment of the variant Cas protein to form a full-length variant Cas protein. In some of any embodiments, wherein the N-terminal Intein comprises an N-terminal Npu Intein, or the sequence set forth in SEQ ID NO:178, or an amino acid sequence that has at least 90%, 91%, 92%, 93%, 94%, 95%, 96%, 97%, 98%, or 99% sequence identity thereto, or a portion of any of the foregoing. In some of any embodiments, the N-terminal fragment of the variant Cas protein comprises: the N-terminal fragment of variant SpCas9 from the N-terminal end up to position 573 of the dSpCas9 sequence set forth in SEQ ID NO:78, or an amino acid sequence that has at least 90%, 91%, 92%, 93%, 94%, 95%, 96%, 97%, 98%, or 99% sequence identity thereto; or the sequence set forth in SEQ ID NO:176, or an amino acid sequence that has at least 90%, 91%, 92%, 93%, 94%, 95%, 96%, 97%, 98%, or 99% sequence identity thereto, or a portion of any of the foregoing. In some of any embodiments, the C-terminal Intein comprises a C-terminal Npu Intein, or the sequence set forth in SEQ ID NO:182, or an amino acid sequence that has at least 90%, 91%, 92%, 93%, 94%, 95%, 96%, 97%, 98%, or 99% sequence identity thereto, or a portion of any of the foregoing. In some of any embodiments, the C-terminal fragment of the variant Cas protein comprises: the C-terminal fragment of variant SpCas9 from position 574 to the C-terminal end of the dSpCas9 sequence set forth in SEQ ID NO:78, or an amino acid sequence that has at least 90%, 91%, 92%, 93%, 94%, 95%, 96%, 97%, 98%, or 99% sequence identity thereto; or the sequence set forth in SEQ ID NO:184, or an amino acid sequence that has at least 90%, 91%, 92%, 93%, 94%, 95%, 96%, 97%, 98%, or 99% sequence identity thereto, or a portion of any of the foregoing.

In some of any embodiments, the regulatory DNA element is an enhancer.

Also provided are DNA-targeting systems comprising a DNA-targeting domain that binds to a target site in a regulatory DNA element of a frataxin (FXN) locus; and at least one effector domain that increases transcription of the FXN locus; wherein the target site is located within the genomic coordinates human genome assembly GRCh38 (hg38) chr9:69,027,282-69,028,497.

In some of any embodiments, the target site is located within the genomic coordinates human genome assembly GRCh38 (hg38) chr9:69,027,282-69,028,497. In some of any embodiments, the target site is located within the genomic coordinates hg38 chr9:69,027,615-69,028,101. In some of any embodiments, the target site comprises the sequence set forth in SEQ ID NO:21, a contiguous portion thereof of at least 14 nt, or a complementary sequence of any of the foregoing.

In some of any embodiments, the DNA-targeting domain comprises a Cas-gRNA combination comprising (a) a Cas protein or a variant thereof and (b) at least one gRNA; and the gRNA comprises at least one gRNA spacer sequence comprising the sequence set forth in SEQ ID NO:42, or a contiguous portion thereof of at least 14 nt. In some of any embodiments, the gRNA further comprises the sequence set forth in SEQ ID NO:44. In some of any embodiments, the gRNA comprises the sequence set forth in SEQ ID NO:67.

In some of any embodiments, the regulatory DNA element is a promoter.

In some of any embodiments, the target site is located within the genomic coordinates hg38 chr9:68,940,179-69,205,519. In some of any embodiments, the target site comprises a sequence selected from any one of SEQ ID NOS:1-10, a contiguous portion thereof of at least 14 nt, or a complementary sequence of any of the foregoing.

In some of any embodiments, the DNA-targeting domain comprises a Cas-gRNA combination comprising (a) a Cas protein or a variant thereof and (b) at least one gRNA; and the gRNA comprises at least one gRNA spacer sequence comprising a sequence selected from any one of SEQ ID NOS:22-31, or a contiguous portion thereof of at least 14 nt. In some of any embodiments, the gRNA further comprises the sequence set forth in SEQ ID NO:44. In some of any embodiments, the gRNA comprises a sequence selected from any one of SEQ ID NOS:47-56. In some embodiments, the gRNA is set forth in SEQ ID NO:47 or 53. In some of any embodiments, the target site comprises a sequence selected from any one of SEQ ID NOS:11-20, a contiguous portion thereof of at least 14 nt, or a complementary sequence of any of the foregoing.

In some of any embodiments, the DNA-targeting domain comprises a Cas-gRNA combination comprising (a) a Cas protein or a variant thereof and (b) at least one gRNA; and the gRNA comprises at least one gRNA spacer sequence comprising a sequence selected from any one of SEQ ID NOS:32-41, or a contiguous portion thereof of at least 14 nt. In some of any embodiments, the target site comprises a sequence selected from any of SEQ ID NOS:12-14 and 16-19, a contiguous portion thereof of at least 14 nt, or a complementary sequence of any of the foregoing. In some of any embodiments, the at least one gRNA comprises a gRNA spacer sequence comprising a sequence selected from any of SEQ ID NOS:33-35 and 37-40, or a contiguous portion thereof of at least 14 nt. In some of any embodiments, the gRNA further comprises the sequence set forth in SEQ ID NO:46. In some of any embodiments, the gRNA comprises a sequence selected from any one of SEQ ID NOS:57-66.

In some of any embodiments, the gRNA comprises a gRNA spacer sequence comprising SEQ ID NO:22, or a contiguous portion thereof of at least 14 nt. In some of any embodiments, the gRNA comprises a gRNA spacer sequence comprising SEQ ID NO:23, or a contiguous portion thereof of at least 14 nt. In some of any embodiments, the gRNA comprises a gRNA spacer sequence comprising SEQ ID NO:24, or a contiguous portion thereof of at least 14 nt. In some of any embodiments, the gRNA comprises a gRNA spacer sequence comprising SEQ ID NO:25, or a contiguous portion thereof of at least 14 nt. In some of any embodiments, the gRNA comprises a gRNA spacer sequence comprising SEQ ID NO:26, or a contiguous portion thereof of at least 14 nt. In some of any embodiments, the gRNA comprises a gRNA spacer sequence comprising SEQ ID NO:27, or a contiguous portion thereof of at least 14 nt. In some of any embodiments, the gRNA comprises a gRNA spacer sequence comprising SEQ ID NO:28, or a contiguous portion thereof of at least 14 nt. In some of any embodiments, the gRNA comprises a gRNA spacer sequence comprising SEQ ID NO:29, or a contiguous portion thereof of at least 14 nt. In some of any embodiments, the gRNA comprises a gRNA spacer sequence comprising SEQ ID NO:30, or a contiguous portion thereof of at least 14 nt.

Also provided herein are DNA-targeting systems comprising a DNA-targeting domain that is a Cas-guide RNA (gRNA) combination comprising: (a) a Staphylococcus aureus deactivated Cas9 protein (dSaCas9) protein set forth in SEQ ID NO:72 fused to a VP16 tetramer (“VP64”); and (b) a gRNA comprising a gRNA spacer sequence set forth in SEQ ID NO:42.

Also provided herein are DNA-targeting systems comprising a DNA-targeting domain that is a Cas-guide RNA (gRNA) combination comprising: (a) a Staphylococcus aureus deactivated Cas9 protein (dSaCas9) protein set forth in SEQ ID NO:72 fused to a VP16 tetramer (“VP64”); and (b) a gRNA comprising a gRNA spacer sequence set forth in SEQ ID NO:22.

Also provided herein are DNA-targeting systems comprising a DNA-targeting domain that is a Cas-guide RNA (gRNA) combination comprising: (a) a Staphylococcus aureus deactivated Cas9 protein (dSaCas9) protein set forth in SEQ ID NO:72 fused to a VP16 tetramer (“VP64”); and (b) a gRNA comprising a gRNA spacer sequence set forth in SEQ ID NO:28.

In some of any embodiments, the gRNA spacer sequence is between 14 nt and 24 nt, or between 16 nt and 22 nt in length. In some of any embodiments, the gRNA spacer sequence is 18 nt, 19 nt, 20 nt, 21 nt or 22 nt in length. In some of any embodiments, the gRNA comprises modified nucleotides for increased stability.

In some of any embodiments, the DNA-targeting system further comprises at least one effector domain. In some of any embodiments, the DNA-targeting domain or a component thereof is fused to the at least one effector domain.

In some of any embodiments, the DNA-targeting domain comprises a Cas-gRNA combination comprising (a) a Cas protein or a variant thereof and (b) at least one gRNA, and the component thereof fused to the at least one effector domain is the Cas protein or a variant thereof.

In some of any embodiments, the effector domain induces, catalyzes or leads to transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation. In some of any embodiments, the effector domain induces, catalyzes or leads to transcription activation, transcription co-activation, transcription elongation. In some of any embodiments, the effector domain induces transcription activation.

Also provided herein are DNA-targeting systems or combinations of DNA-targeting systems comprising a DNA-targeting domain, that binds to a target site in a regulatory DNA element of a frataxin (FXN) locus and comprises a Cas-guide RNA (gRNA) combination comprising: (a) a Staphylococcus aureus deactivated Cas9 protein (dSaCas9) protein set forth in SEQ ID NO:72 fused to at least one effector domain that induces transcription activation; and (b) at least one gRNA comprising at least one gRNA spacer sequence set forth in SEQ ID NO:42.

Also provided herein are DNA-targeting systems or combinations of DNA-targeting systems comprising a DNA-targeting domain, that binds to a target site in a regulatory DNA element of a frataxin (FXN) locus and comprises a Cas-guide RNA (gRNA) combination comprising: (a) a Staphylococcus aureus deactivated Cas9 protein (dSaCas9) protein set forth in SEQ ID NO:72 fused to at least one effector domain that induces transcription activation; and (b) at least one gRNA comprising at least one gRNA spacer sequence set forth in SEQ ID NO:22.

Also provided herein are DNA-targeting systems or combinations of DNA-targeting systems comprising a DNA-targeting domain, that binds to a target site in a regulatory DNA element of a frataxin (FXN) locus and comprises a Cas-guide RNA (gRNA) combination comprising: (a) a Staphylococcus aureus deactivated Cas9 protein (dSaCas9) protein set forth in SEQ ID NO:72 fused to at least one effector domain that induces transcription activation; and (b) at least one gRNA comprising at least one gRNA spacer sequence set forth in SEQ ID NO:28.

In some of any embodiments, the effector domain comprises at least one VP16 domain, or a VP16 tetramer (“VP64”) or a variant thereof. In some of any embodiments, the effector domain comprises the sequence set forth in SEQ ID NO:81 or 83, or a portion thereof, or an amino acid sequence that has at least 90%, 91%, 92%, 93%, 94%, 95%, 96%, 97%, 98%, or 99% sequence identity to any of the foregoing.

In some of any embodiments, the effector domain is selected from a p65 activation domain, a p300 domain, DPOLA, ENL, FOXO3, HSH2D, NCOA2, NCOA3, PSA1, PYGO1, RBM39, HERC2, DMD, or NOTCH2, or a domain thereof, a portion thereof or a variant thereof. In some of any embodiments, the effector domain comprises a sequence selected from any one of SEQ ID NOS: 113-125, or a domain thereof, a portion thereof, or an amino acid sequence that has at least 90%, 91%, 92%, 93%, 94%, 95%, 96%, 97%, 98%, or 99% sequence identity to any of the foregoing. In some of any embodiments, the effector domain comprises a sequence selected from any one of SEQ ID NOS: 100-112, or a domain thereof, a portion thereof, or an amino acid sequence that has at least 90%, 91%, 92%, 93%, 94%, 95%, 96%, 97%, 98%, or 99% sequence identity to any of the foregoing. In some of any embodiments, the DNA targeting system or variant thereof comprises a truncation thereof.

In some of any embodiments, the at least one effector domain is fused to the N-terminus, the C-terminus, or both the N-terminus and the C-terminus, of the DNA-targeting domain or a component thereof. In some of any embodiments, the DNA-targeting system further comprises one or more linkers connecting the DNA-targeting domain or a component thereof to the at least one effector domain, and/or further comprising one or more nuclear localization signals (NLS).

In some of any embodiments, the DNA-targeting system comprises the sequence set forth in SEQ ID NO:71, or an amino acid sequence that has at least 90%, 91%, 92%, 93%, 94%, 95%, 96%, 97%, 98%, or 99% sequence identity thereto. In some of any embodiments, the DNA-targeting system comprises the sequence set forth in SEQ ID NO:77, or an amino acid sequence that has at least 90%, 91%, 92%, 93%, 94%, 95%, 96%, 97%, 98%, or 99% sequence identity thereto.

In some of any embodiments, the DNA-targeting domain is a first DNA-targeting domain, and the DNA-targeting system further comprises one or more second DNA-targeting domains. In some of any embodiments, the first DNA-targeting domain binds a first target site in an enhancer of a FXN locus, and the second DNA-targeting domain binds a second target site in a promoter of a FXN locus.

Also provided herein are DNA-targeting systems that binds to one or more target sites in a regulatory DNA element of a frataxin (FXN) locus, the DNA-targeting system comprising: a first DNA-targeting domain that binds a first target site in an enhancer of a FXN locus, and a second DNA-targeting domain that binds a second target site in a promoter of a FXN locus.

Also provided herein are combinations that comprise a first DNA-targeting domain comprising any of the DNA targeting domain provided herein, and one or more second DNA-targeting domains. In some embodiments, the one or more second DNA-targeting domains comprises any of the DNA targeting domain provided herein. In some of any embodiments, the first DNA-targeting domain binds a first target site in an enhancer of a FXN locus; and the second DNA-targeting domain binds a second target site in a promoter of a FXN locus.

Also provided herein are combinations that comprise a first DNA-targeting domain that binds a first target site in an enhancer of a frataxin (FXN) locus; and a second DNA-targeting domain that binds a second target site in a promoter of a FXN locus.

In some of any embodiments, the first target site is located within the genomic coordinates human genome assembly GRCh38 (hg38) chr9:69,027,282-69,028,497, and the second target site is located within the genomic coordinates hg38 chr9:68,940,179-69,205,519. In some of any embodiments, the first target site is located within the genomic coordinates hg38 chr9:69,027,615-69,028,101, and the second target site is located within the genomic coordinates hg38 chr9:68,940,179-69,205,519.

In some of any embodiments, the first DNA-targeting domain comprises a first Cas-gRNA combination comprising (a) a first Cas protein or a variant thereof and (b) a first gRNA that is capable of hybridizing to the target site or is complementary to the first target site; and the second DNA-targeting domain comprises a second Cas-gRNA combination comprising (a) a second Cas protein or a variant thereof and (b) a second gRNA that is capable of hybridizing to the target site or is complementary to the second target site. In some of any embodiments, the first DNA-targeting domain comprises a first Cas-gRNA combination comprising (a) a first Cas protein or a variant thereof and (b) a first gRNA comprising at least one gRNA spacer sequence set forth in SEQ ID NO:42 or a contiguous portion thereof of at least 14 nt. In some of any embodiments, the second DNA-targeting domain comprises a second Cas-gRNA combination comprising (a) a second Cas protein or a variant thereof and (b) a second gRNA comprising at least one gRNA spacer sequence set forth in SEQ ID NO:22 or 28 or a contiguous portion thereof of at least 14 nt.

In some of any embodiments, the first Cas-gRNA combination comprises (a) a first Cas protein or a variant thereof and (b) a first gRNA comprising at least one gRNA spacer sequence set forth in SEQ ID NO:42 or a contiguous portion thereof of at least 14 nt; and the second Cas-gRNA combination comprises (a) a second Cas protein or a variant thereof and (b) a second gRNA comprising at least one gRNA spacer sequence set forth in SEQ ID NO:22 or a contiguous portion thereof of at least 14 nt. In some of any embodiments, the first Cas-gRNA combination comprises (a) a first Cas protein or a variant thereof and (b) a first gRNA comprising at least one gRNA spacer sequence set forth in SEQ ID NO:42 or a contiguous portion thereof of at least 14 nt; and the second Cas-gRNA combination comprises (a) a second Cas protein or a variant thereof and (b) a second gRNA comprising at least one gRNA spacer sequence set forth in SEQ ID NO:28 or a contiguous portion thereof of at least 14 nt.

In some of any embodiments, the first DNA-targeting domain binds a first target site in a promoter of a FXN locus; and the second DNA-targeting domain binds a second target site in a promoter of a FXN locus.

Also provided herein are DNA-targeting systems that binds to one or more target sites in a regulatory DNA element of a frataxin (FXN) locus, the DNA-targeting system comprising: a first DNA-targeting domain that binds a first target site in a promoter of a FXN locus; and a second DNA-targeting domain that binds a second target site in a promoter of a FXN locus.

Also provided herein are combinations that comprise a first DNA-targeting domain that binds a first target site in a promoter of a frataxin (FXN) locus; and a second DNA-targeting domain that binds a second target site in a promoter of a FXN locus.

In some of any embodiments, the first target site and the second target site independently are located within the genomic coordinates hg38 chr9:68,940,179-69,205,519. In some of any embodiments, the first target site and the second target site are different.

In some of any embodiments, the first DNA-targeting domain comprises a first Cas-gRNA combination comprising (a) a first Cas protein or a variant thereof and (b) a first gRNA that is capable of hybridizing to the target site or is complementary to the first target site; and the second DNA-targeting domain comprises a second Cas-gRNA combination comprising (a) a second Cas protein or a variant thereof and (b) a second gRNA that is capable of hybridizing to the target site or is complementary to the second target site. In some of any embodiments, the first DNA-targeting domain comprises a first Cas-gRNA combination comprising (a) a first Cas protein or a variant thereof and (b) a first gRNA comprising at least one gRNA spacer sequence set forth in SEQ ID NO:22 or a contiguous portion thereof of at least 14 nt. In some of any embodiments, the second DNA-targeting domain comprises a second Cas-gRNA combination comprising (a) a second Cas protein or a variant thereof and (b) a second gRNA comprising at least one gRNA spacer sequence set forth in SEQ ID NO:28 or a contiguous portion thereof of at least 14 nt.

In some of any embodiments, the first Cas-gRNA combination comprises (a) a first Cas protein or a variant thereof and (b) a first gRNA comprising at least one gRNA spacer sequence set forth in SEQ ID NO:22 or a contiguous portion thereof of at least 14 nt; and the second Cas-gRNA combination comprises (a) a second Cas protein or a variant thereof and (b) a second gRNA comprising at least one gRNA spacer sequence set forth in SEQ ID NO:28 or a contiguous portion thereof of at least 14 nt.

In some of any embodiments, the first Cas protein or a variant thereof and/or the second Cas protein or a variant thereof is a variant Cas9 protein that lacks nuclease activity or that is a deactivated Cas9 (dCas9) protein.

In some of any embodiments, the first variant Cas protein and/or the second variant Cas protein is a Staphylococcus aureus dCas9 protein (dSaCas9) that comprises at least one amino acid mutation selected from D10A and N580A, with reference to numbering of positions of SEQ ID NO:73; or comprises the sequence set forth in SEQ ID NO:72, or an amino acid sequence that has at least 90%, 91%, 92%, 93%, 94%, 95%, 96%, 97%, 98%, or 99% sequence identity thereto.

In some of any embodiments, the first variant Cas protein and/or the second variant Cas protein is a Streptococcus pyogenes dCas9 (dSpCas9) protein that comprises at least one amino acid mutation selected from D10A and H840A, with reference to numbering of positions of SEQ ID NO:79; or comprises the sequence set forth in SEQ ID NO:78, or an amino acid sequence that has at least 90%, 91%, 92%, 93%, 94%, 95%, 96%, 97%, 98%, or 99% sequence identity thereto. In some of any embodiments, the first Cas protein and the second Cas protein are the same. In some of any embodiments, the first Cas protein and the second Cas protein are different.

In some of any embodiments, the first Cas protein or a variant thereof and/or the second Cas protein or a variant thereof is fused to at least one effector domain.

In some of any embodiments, the effector domain induces, catalyzes or leads to transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation. In some of any embodiments, the effector domain induces transcription activation.

In some of any embodiments, the first DNA-targeting domain and the second DNA-targeting domain are encoded in a first polynucleotide. In some of any embodiments, the first Cas protein and the second Cas protein are encoded in a first polynucleotide. In some of any embodiments, the first Cas protein and the second Cas protein are encoded by the same nucleotide sequence. In some of any embodiments, the first gRNA and the second gRNA are encoded in a first polynucleotide. In some of any embodiments, the first Cas protein and the second Cas protein are encoded by the same nucleotide sequence, and the Cas protein, the first gRNA, and the second gRNA are encoded in a first polynucleotide. In some of any embodiments, the first DNA-targeting domain is encoded in a first polynucleotide and the second DNA-targeting domain is encoded in a second polynucleotide. In some of any embodiments, the first Cas protein is encoded in a first polynucleotide and the second Cas protein is encoded in a second polynucleotide. In some of any embodiments, the first gRNA is encoded in a first polynucleotide and the second gRNA is encoded in a second polynucleotide. In some of any embodiments, the first Cas protein and the first gRNA are encoded in a first polynucleotide, and the second Cas protein and the second gRNA are encoded in a second polynucleotide.

Also provided herein is a guide RNA (gRNA) that binds a target site in an enhancer region of a frataxin (FXN) locus, wherein the target site is located within the genomic coordinates human genome assembly GRCh38 (hg38) chr9:69,027,282-69,028,497.

In some of any provided embodiments, the target site is located within the genomic coordinates hg38 chr9:69,027,615-69,028,101. In some of any provided embodiments, the target site comprises the sequence set forth in SEQ ID NO:21, a contiguous portion thereof of at least 14 nt, or a complementary sequence of any of the foregoing. In some of any provided embodiments, the gRNA comprises at least one gRNA spacer comprising the sequence set forth in SEQ ID NO:42, or a contiguous portion thereof of at least 14 nt. In some of any provided embodiments, the gRNA further comprises the sequence set forth in SEQ ID NO:44. In some of any provided embodiments, the gRNA comprises the sequence set forth in SEQ ID NO:67.

Also provided herein is a guide RNA (gRNA) that binds a target site in a regulatory DNA element of a frataxin (FXN) locus wherein the target site comprises a sequence selected from any one of SEQ ID NOS: 1-10, a contiguous portion thereof of at least 14 nt, or a complementary sequence of any of the foregoing.

In some of any provided embodiments, the gRNA comprises at least one gRNA spacer sequence comprising a sequence selected from any one of SEQ ID NOS:22-31, or a contiguous portion thereof of at least 14 nt. In some of any provided embodiments, the gRNA further comprises the sequence set forth in SEQ ID NO:44. In some of any provided embodiments, the gRNA comprises a sequence selected from any one of SEQ ID NOS:47-56.

Also provided herein is a guide RNA (gRNA) that binds a target site in a regulatory DNA element of a frataxin (FXN) locus wherein the target site comprises a sequence selected from any one of SEQ ID NOS: 11-20, a contiguous portion thereof of at least 14 nt, or a complementary sequence of any of the foregoing.

In some of any provided embodiments, the gRNA comprises at least one gRNA spacer sequence comprising a sequence selected from any one of SEQ ID NOS:32-41, or a contiguous portion thereof of at least 14 nt. In some of any provided embodiments, the gRNA further comprises the sequence set forth in SEQ ID NO:46. In some of any provided embodiments, the gRNA comprises a sequence selected from any one of SEQ ID NOS:57-66. In some of any provided embodiments, the gRNA spacer sequence is between 14 nt and 24 nt, or between 16 nt and 22 nt in length. In some of any provided embodiments, the gRNA spacer sequence is 18 nt. 19 nt, 20 nt, 21 nt or 22 nt in length. In some of any provided embodiments, the gRNA comprises modified nucleotides for increased stability. In some of any provided embodiments, the gRNA is capable of complexing with the Cas protein or variant thereof. In some of any provided embodiments, the gRNA comprises at least one gRNA spacer sequence that is capable of hybridizing to the target site or is complementary to the target site.

Also provided herein is a combination, comprising a first gRNA comprising any of the gRNAs described herein, and one or more second gRNAs that binds to a second target site in a regulatory DNA element of a frataxin (FXN) locus.

In some of any embodiments, the second gRNA comprises any of the gRNAs described herein.

Also provided herein is a combination, comprising: a first gRNA that binds a first target site in an enhancer region of a frataxin (FXN) locus, wherein the first target site is located within the genomic coordinates human genome assembly GRCh38 (hg38) chr9:69,027,282-69,028,497; and a second gRNA that binds a second target site in a promoter region of a FXN locus, wherein the second target site is located within the genomic coordinates hg38 chr9:68,940,179-69,205,519.

In some of any embodiments, the first gRNA comprises at least one gRNA spacer sequence set forth in SEQ ID NO:42 or a contiguous portion thereof of at least 14 nt. In some of any embodiments, the second gRNA comprises at least one gRNA spacer sequence set forth in SEQ ID NO:22 or 28 or a contiguous portion thereof of at least 14 nt. In some of any of the provided embodiments, for example of a combination provided herein: the first gRNA comprises at least one gRNA spacer sequence set forth in SEQ ID NO:42 or a contiguous portion thereof of at least 14 nt; and the second gRNA comprises at least one gRNA spacer sequence set forth in SEQ ID NO:22 or a contiguous portion thereof of at least 14 nt. In some of any of the provided embodiments, for example of a combination provided herein: the first gRNA comprises at least one gRNA spacer sequence set forth in SEQ ID NO:42 or a contiguous portion thereof of at least 14 nt; and the second gRNA comprises at least one gRNA spacer sequence set forth in SEQ ID NO:28 or a contiguous portion thereof of at least 14 nt.

Also provided herein is a combination, comprising: a first gRNA that binds a first target site in a promoter region of a FXN locus, wherein the second target site is located within the genomic coordinates hg38 chr9:68,940,179-69,205,519; and a second gRNA that binds a second target site in a promoter region of a frataxin (FXN) locus, wherein the first target site is located within the genomic coordinates hg38 chr9:68,940,179-69,205,519.

In some of any embodiments, the combination comprises: the first gRNA comprises at least one gRNA spacer sequence set forth in SEQ ID NO:22 or a contiguous portion thereof of at least 14 nt; and the second gRNA comprises at least one gRNA spacer sequence set forth in SEQ ID NO:28 or a contiguous portion thereof of at least 14 nt.

Also provided herein is a fusion protein comprising (1) a DNA-targeting domain or a component thereof and (2) at least one effector domain, wherein: the DNA-targeting domain or a component thereof binds to a target site in a regulatory DNA element of a frataxin (FXN) locus; and the effector domain induces, catalyzes or leads to transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation.

In some of any embodiments, the DNA-targeting domain comprises a Clustered Regularly Interspaced Short Palindromic Repeats associated (Cas)-guide RNA (gRNA) combination comprising (a) a Cas protein or a variant thereof and (b) at least one gRNA; a zinc finger protein (ZFP); a transcription activator-like effector (TALE); a meganuclease; a homing endonuclease; or a I-SceI enzymes or a variant thereof, such as a catalytically inactive variant thereof.

In some of any embodiments, the DNA-targeting domain comprises a Cas-gRNA combination comprising a Cas protein or a variant thereof and at least one gRNA, and the component of the DNA-targeting domain is a Cas protein or a variant thereof. In some of any embodiments, the Cas protein or a variant thereof is capable of complexing with at least one gRNA. In some embodiments, the gRNA binds to a target site in a regulatory DNA element of a frataxin (FXN) locus. In some of any embodiments, binding of the DNA-targeting domain or a component thereof to the target site does not introduce a genetic disruption or a DNA break at or near the target site. In some of any embodiments, the variant Cas protein lacks nuclease activity or is a deactivated Cas (dCas) protein.

In some of any embodiments, the gRNA is capable of complexing with the Cas protein or variant thereof. In some of any embodiments, the Cas protein or a variant thereof is a Cas9 protein or a variant thereof. In some of any embodiments, the variant Cas protein is a variant Cas9 protein that lacks nuclease activity or that is a deactivated Cas9 (dCas9) protein.

In some of any embodiments, the Cas9 protein or a variant thereof is a Staphylococcus aureus Cas9 (SaCas9) protein or a variant thereof. In some of any embodiments, the variant Cas9 is a Staphylococcus aureus dCas9 protein (dSaCas9) that comprises at least one amino acid mutation selected from D10A and N580A, with reference to numbering of positions of SEQ ID NO:73. In some of any embodiments, the variant Cas9 protein comprises the sequence set forth in SEQ ID NO:72, or an amino acid sequence that has at least 90%, 91%, 92%, 93%, 94%, 95%, 96%, 97%, 98%, or 99% sequence identity thereto.

In some of any embodiments, the Cas9 protein or variant thereof is a Streptococcus pyogenes Cas9 (SpCas9) protein or a variant thereof. In some of any embodiments, the variant Cas9 is a Streptococcus pyogenes dCas9 (dSpCas9) protein that comprises at least one amino acid mutation selected from D10A and H840A, with reference to numbering of positions of SEQ ID NO:79. In some of any embodiments, the variant Cas9 protein comprises the sequence set forth in SEQ ID NO:78, or an amino acid sequence that has at least 90%, 91%, 92%, 93%, 94%, 95%, 96%, 97%, 98%, or 99% sequence identity thereto.

In some of any embodiments, the regulatory DNA element is an enhancer. In some of any embodiments, the target site is located within the genomic coordinates human genome assembly GRCh38 (hg38) chr9:69,027,282-69,028,497. In some of any embodiments, the target site is located within the genomic coordinates hg38 chr9:69,027,615-69,028,101. In some of any embodiments, the target site comprises the sequence set forth in SEQ ID NO:21, a contiguous portion thereof of at least 14 nt, or a complementary sequence of any of the foregoing.

In some of any embodiments, the regulatory DNA element is a promoter. In some of any embodiments, the target site is located within the genomic coordinates hg38 chr9:68,940,179-69,205,519. In some of any embodiments, the target site comprises a sequence selected from any one of SEQ ID NOS: 1-10, a contiguous portion thereof of at least 14 nt, or a complementary sequence of any of the foregoing. In some of any embodiments, the target site comprises a sequence selected from any one of SEQ ID NOS: 11-20, a contiguous portion thereof of at least 14 nt, or a complementary sequence of any of the foregoing.

In some of any embodiments, the effector domain induces, catalyzes or leads to transcription activation, transcription co-activation, transcription elongation. In some of any embodiments, the effector domain induces transcription activation. In some of any embodiments, the effector domain comprises at least one VP16 domain, or a VP16 tetramer (“VP64”) or a variant thereof. In some of any embodiments, the effector domain comprises the sequence set forth in SEQ ID NO:81 or 83, or a portion thereof, or an amino acid sequence that has at least 90%, 91%, 92%, 93%, 94%, 95%, 96%, 97%, 98%, or 99% sequence identity to any of the foregoing.

In some of any embodiments, the effector domain is selected from a p65 activation domain, a p300 domain, DPOLA, ENL, FOXO3, HSH2D, NCOA2, NCOA3, PSA1, PYGO1, RBM39, HERC2, DMD, or NOTCH2, or a domain thereof, a portion thereof or a variant thereof. In some of any embodiments, the effector domain comprises a sequence selected from any one of SEQ ID NOS: 113-125, or a domain thereof, a portion thereof, or an amino acid sequence that has at least 90%, 91%, 92%, 93%, 94%, 95%, 96%, 97%, 98%, or 99% sequence identity to any of the foregoing. In some of any embodiments, the effector domain comprises a sequence selected from any one of SEQ ID NOS: 100-112, or a domain thereof, a portion thereof, or an amino acid sequence that has at least 90%, 91%, 92%, 93%, 94%, 95%, 96%, 97%, 98%, or 99% sequence identity to any of the foregoing. In some of any embodiments, the variant thereof comprises a truncation thereof.

In some of any embodiments, the at least one effector domain is fused to the N-terminus, the C-terminus, or both the N-terminus and the C-terminus, of the DNA-targeting domain or a component thereof. In some of any embodiments, the fusion protein further comprises one or more linkers connecting the DNA-targeting domain or a component thereof to the at least one effector domain, and/or further comprising one or more nuclear localization signals (NLS). In some of any embodiments, the fusion protein comprises the sequence set forth in SEQ ID NO:71, or an amino acid sequence that has at least 90%, 91%, 92%, 93%, 94%, 95%, 96%, 97%, 98%, or 99% sequence identity thereto. In some of any embodiments, the fusion protein comprises the sequence set forth in SEQ ID NO:77, or an amino acid sequence that has at least 90%, 91%, 92%, 93%, 94%, 95%, 96%, 97%, 98%, or 99% sequence identity thereto.

Also provided herein are polynucleotides encoding any of the DNA-targeting systems described herein, any of the gRNAs described herein, any of the combinations described herein, or any of the fusion proteins described herein, or a portion or a component of any of the foregoing.

Also provided herein are polynucleotides encoding a first DNA-targeting system, a first Cas protein and/or a first gRNA of any of the DNA-targeting systems described herein or any of the combinations described herein.

Also provided herein are polynucleotides encoding a second DNA-targeting system, a second Cas protein and/or a second gRNA of any of the DNA-targeting systems described herein or any of the combinations described herein.

Also provided herein are pluralities of polynucleotides, comprising any of the polynucleotides described herein, and one or more additional polynucleotides encoding an additional portion or an additional component of any of the DNA-targeting systems described herein, any of the gRNAs described herein, any of the combinations described herein, or any of the fusion proteins described herein, or a portion or a component of any of the foregoing.

Also provided herein are pluralities of polynucleotides, comprising: (a) a polynucleotide encoding a first DNA-targeting system, a first Cas protein and/or a first gRNA of any of the a DNA-targeting system disclosed herein or any of the combinations of gRNAs disclosed herein, and (b) a polynucleotide encoding a second DNA-targeting system, a second Cas protein and/or a second gRNA of any of the a DNA-targeting system disclosed herein or any of the combinations of gRNAs disclosed herein.

Also provided herein are vectors comprising any of the polynucleotides described herein, any of the pluralities of polynucleotides described herein, or a first polynucleotide or a second polynucleotide of any of the pluralities of polynucleotides described herein, or a portion or a component of any of the foregoing.

In some of any of the provided embodiments, the vector is a viral vector. In some of any of the provided embodiments, the viral vector is an AAV vector. In some of any of the provided embodiments, the AAV vector is engineered for central nervous system (CNS) tropism. In some of any of the provided embodiments, the AAV vector is selected from among AAV1, AAV2, AAV3, AAV4, AAV5, AAV6, AAV7, AAV8, AAV9, AAV10, AAV11, AAV12, or AAV-DJ. In some of any of the embodiments, the AAV vector is AAV9. In some of any of the provided embodiments, the vector is a non-viral vector selected from: a lipid nanoparticle, a liposome, an exosome, or a cell penetrating peptide

Also provided herein are pluralities of vectors, comprising any of the vectors described herein, and one or more additional vectors comprising one or more additional polynucleotides encoding an additional portion or an additional component of any of the DNA-targeting systems described herein, any of the gRNAs described herein, any of the combinations described herein, or any of the fusion proteins described herein, or a portion or a component of any of the foregoing.

Also provided herein are pluralities of vectors, comprising: a first vector comprising a polynucleotide encoding a first DNA-targeting system, a first Cas protein and/or a first gRNA of any of a DNA-targeting system described herein or any of the combinations of gRNAs described herein; and; a second vector comprising a polynucleotide encoding a second DNA-targeting system, a second Cas protein and/or a second gRNA of any of a DNA-targeting system described herein or any of the combinations of gRNAs described herein.

Also provided are combinations, comprising any of the fusion proteins described herein and at least one gRNA. In some of any embodiments, the at least one gRNA is any of the gRNAs described herein.

Also provided herein are cells comprising any of the DNA-targeting systems described herein, any of the gRNAs described herein, any of the combinations described herein, any of the fusion proteins described herein, any of the polynucleotides described herein, any of the pluralities of polynucleotides described herein, any of the vectors described herein, any of the pluralities of vectors described herein, or a portion or a component of any of the foregoing.

In some of any of the embodiments, the cell is a heart cell, such as a cardiomyocyte, a skeletal muscle cell, a nervous system cell, such as a neuron, a fibroblast, or an induced pluripotent stem cell (iPSC). In some of any of the embodiments, the cell is a cardiomyocyte. In some of any of the embodiments, the cell is a neuron. In some of any of the embodiments, the cell is a fibroblast. In some of any of the embodiments, the cell is an iPSC.

In some of any of the embodiments, the cell is from a subject that has or is suspected of having Friedreich's ataxia (FA). In some embodiments, the cell exhibits reduced expression of FXN mRNA. In some embodiments, the cell exhibits reduced expression of FXN protein. In some embodiments, the cell has a GAA trinucleotide repeat expansion in the FXN gene. Also provided herein are methods for modulating the expression of frataxin (FXN) in a cell, the method comprising: introducing any of the DNA-targeting systems described herein, any of the gRNAs described herein, any of the combinations described herein, any of the fusion proteins described herein, any of the polynucleotides described herein, any of the pluralities of polynucleotides described herein, any of the vectors described herein, any of the pluralities of vectors described herein, or a portion or a component of any of the foregoing, into the cell.

Also provided herein are methods of inducing a genetic disruption at a target site in an enhancer region of a frataxin (FXN) locus in a cell, wherein the target site is located within the genomic coordinates human genome assembly GRCh38 (hg38) chr9:69,027,282-69,028,497, the method comprising: contacting a cell with any of the DNA-targeting systems described herein, any of the gRNAs described herein, any of the combinations described herein, any of the fusion proteins described herein, any of the polynucleotides described herein, any of the plurality of polynucleotides described herein, any of the vectors described herein, any of the pluralities of vectors described herein, or a portion or a component of any of the foregoing.

In some of any embodiments, the cell is from a subject that has or is suspected of having Friedreich's ataxia (FA).

Also provided herein are methods for modulating the expression of frataxin (FXN) in a subject, the method comprising: administering any of the DNA-targeting systems described herein, any of the gRNAs described herein, any of the combinations of gRNAs described herein, any of the fusion proteins described herein, any of the polynucleotides described herein, any of the pluralities of polynucleotides described herein, any of the vectors described herein, any of the plurality of vectors described herein, or a portion or a component of any of the foregoing, to the subject.

In some of any embodiments, the subject has or is suspected of having Friedreich's ataxia (FA).

Also provided herein are methods of treating Friedreich's ataxia (FA), the method comprising: administering any of the DNA-targeting systems described herein, any of the gRNAs described herein, any of the combinations described herein, any of the fusion proteins described herein, any of the polynucleotides described herein, any of the pluralities of polynucleotides described herein, any of the vectors described herein, any of the pluralities of vectors described herein, or a portion or a component of any of the foregoing, to a subject that has or is suspected of having FA.

In some of any embodiments, a cell in the subject exhibits reduced expression of FXN compared to a cell from a normal subject. In some of any embodiments, a cell in the subject has a GAA trinucleotide repeat expansion in the FXN gene. In some of any embodiments, the cell is a heart cell, a skeletal muscle cell, a nervous system cell, or an induced pluripotent stem cell. In some of any embodiments, the introducing, contacting or administering is carried out in vivo or ex vivo. In some of any embodiments, following the introducing, contacting or administering, the expression of frataxin (FXN) is increased in the cell or the subject. In some of any embodiments, the expression of frataxin (FXN) is increased at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold. In some of any embodiments, the expression is increased by less than about 10-fold, 9-fold, 8-fold, 7-fold or 6-fold. In some of any embodiments, the subject is a human.

Also provided herein is a pharmaceutical composition comprising any of the DNA-targeting systems described herein, any of the gRNAs described herein, any of the combinations described herein, any of the fusion proteins described herein, any of the polynucleotides described herein, any of the pluralities of polynucleotides described herein, any of the vectors described herein, any of the pluralities of vectors described herein, or a portion or a component of any of the foregoing.

In some of any of the provided embodiments, the pharmaceutical composition is for use in treating Friedreich's ataxia (FA). In some of any of the provided embodiments, the pharmaceutical composition is for use in the manufacture of a medicament for treating Friedreich's ataxia (FA).

In some of any of the provided embodiments, the pharmaceutical composition is to be administered to a subject.

Also provided herein is the use of any of the pharmaceutical compositions described herein for treating Friedreich's ataxia (FA).

Also provided herein is the use of any of the pharmaceutical compositions described herein in the manufacture of a medicament for treating Friedreich's ataxia (FA).

In some of any of the provided embodiments, the pharmaceutical composition is to be administered to a subject.

In some of any embodiments, the subject has or is suspected of having Friedreich's ataxia (FA). In some of any embodiments, a cell in the subject exhibits reduced expression of FXN compared to a cell from a normal subject. In some of any embodiments, a cell in the subject has a GAA trinucleotide repeat expansion in the FXN gene.

In some of any embodiments, the cell is a heart cell, a skeletal muscle cell, a nervous system cell, or an induced pluripotent stem cell. In some of any embodiments, the administration is carried out in vivo or ex vivo.

In some of any embodiments, following the administration, the expression of frataxin (FXN) is increased in the cell or the subject. In some of any embodiments, the expression is increased at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold. In some of any embodiments, the expression is increased by less than about 10-fold, 9-fold, 8-fold, 7-fold or 6-fold.

In some of any embodiments, the subject is a human.

### BRIEF DESCRIPTION OF THE DRAWINGS

FIGS. 1A-1C show CRISPR-Cas mediated transcriptional activation of human frataxin in WT-iPSCs using dSaCas9-2× VP64 or dSpCas9-2× VP64 and gRNAs targeted to the frataxin promoter. FIG. 1A shows target locations of gRNAs from Table E1, targeted to human frataxin gene promoter (Homo sapiens (human) genome assembly GRCh38 (hg38) chr9:69034622-69036670). FIGS. 1B and 1C show expression of frataxin mRNA following transduction of WT-iPSCs with indicated gRNA and dSaCas9-2× VP64 (FIG. 1B) or dSpCas9-2× VP64 (FIG. 1C), as assessed by RT-qPCR.

FIG. 2 shows CRISPR-Cas mediated transcriptional activation of human frataxin in iPSCs generated from Friedreich's ataxia patients (FA-iPSCs) using dSaCas9-2× VP64 and promoter-targeting gRNA A or gRNA G, as assessed by RT-qPCR. Cell lines harbored expanded GAA trinucleotide repeats (left panel: 604/734 repeats; right panel: 867/867 repeats).

FIG. 3 shows frataxin protein expression levels in WT-iPSCs and FA-iPSCs as assessed by flow cytometry with a mouse monoclonal anti-frataxin AlexaFluor488-conjugated antibody (abcam ab156033). FA-iPSCs exhibit reduced frataxin expression in comparison to WT-iPSC control cells.

FIG. 4 shows scatterplot of results from sequencing analysis for screen of frataxin-saturating gRNA library. WT-iPSCs expressing dSaCas9-2× VP64 were transduced with pooled library of gRNAs and subsequently sorted by flow cytometry into populations representing top 10% and bottom 10% of cells based on frataxin protein expression. Populations were sequenced to identify gRNAs enriched in the frataxin-high population based on DESeq2. Each dot in the scatterplot represents a single gRNA. y-axis represents log fold change in abundance of gRNA in frataxin-high versus frataxin-low population. x-axis represents mean of normalized counts. The promoter-targeting gRNA A and enhancer-targeting gRNA U (indicated in the figures) were significantly enriched in the frataxin-high population.

FIG. 5 shows locations of promoter-targeting gRNA A and enhancer-targeting gRNA U with respect to the frataxin gene and indicators of chromatin state H3K4Me1, H3K4Me3, H3K27Ac, and DNase I hypersensitivity based on data from the Encyclopedia of DNA Elements (ENCODE). gRNA U targets a region (hg38 chr9:69,027,282-69,028,497) exhibiting hallmark chromatin signatures of an enhancer element, including H3K4Me1 and DNase I hypersensitivity.

FIG. 6 shows CRISPR-Cas mediated transcriptional activation of frataxin in iPSCs, using dSaCas9-2× VP64 and promoter-targeting gRNA A or enhancer-targeting gRNA U compared to control gRNA or empty gRNA vector control, as assessed by RT-qPCR. iPSCs harbored normal GAA trinucleotide repeat lengths.

FIGS. 7A-7F show combinatorial modulation of frataxin mRNA expression with promoter-targeting (gRNA A and gRNA G) and enhancer-targeting (gRNA U) gRNAs, as assessed by RT-qPCR. WT-iPSCs or FA-iPSCs with short or long trinucleotide repeats were used to create stable cell lines expressing promoter-targeting gRNA A or gRNA G. Cell lines were transduced with different combinations of dSaCas9-2× VP64 and gRNA, as indicated. Results are shown for cell lines WT-gRNA A-iPSC (FIG. 7A), WT-gRNA G-iPSC (FIG. 7B), FA(short)-gRNA A-iPSC (FIG. 7C), FA(short)-gRNA G-iPSC (FIG. 7D), FA(long)-gRNA A-iPSC (FIG. 7E), and FA(long)-gRNA G-iPSC (FIG. 7F).

FIG. 8 shows the rescue of frataxin mRNA expression with promoter- and enhancer-targeting gRNAs in FA-iPSCs, with frataxin expression levels compared to WT-iPSCs, as assessed by RT-qPCR. FA-iPSCs stably expressing promoter-targeting gRNA A or gRNA G were transduced with different combinations of dSaCas9-2× VP64 and gRNA, as indicated.

FIGS. 9A-9C show CRISPR-Cas mediated activation of frataxin protein expression using promoter- and enhancer-targeting gRNAs, as assessed by ELISA. FIG. 9A shows frataxin protein expression in WT-iPSCs stably expressing gRNA G (WT-gRNA G-iPSCs), which were (a) left untreated, (b) transduced with dSaCas9-2× VP64 and a control gRNA, or (c) transduced with dSaCas9-2× VP64 and the frataxin enhancer-targeting gRNA U. FIGS. 9B and 9C show rescue of frataxin protein expression in FA-iPSCs harboring short (FIG. 9B) or long (FIG. 9C) expanded trinucleotide GAA repeats in comparison to WT-iPSCs. FA-iPSCs stably expressing promoter-targeting gRNAs were transduced with different combinations of dSaCas9-2× VP64 and gRNA, as indicated.

FIGS. 10A and 10B show scatterplots of results from sequencing analysis for screen of dSaCas9 transcription activator and repressor fusion proteins. WT-iPSCs expressing frataxin promoter-targeting gRNA A were transduced with pooled libraries of fusion proteins comprising fragments of nuclear localized proteins, fused to the N-terminus (FIG. 10A) or C-terminus (FIG. 10B) of dSaCas9. Transduced cells were subsequently sorted by flow cytometry into populations representing top 10% and bottom 10% of cells based on frataxin protein expression. Populations were sequenced to identify effectors enriched in the frataxin-high or frataxin-low populations based on DESeq2. Each dot in the scatterplots represents a single effector. y-axis represents log fold change in frataxin-high versus frataxin-low populations. x-axis represents mean of normalized counts. Enriched effectors are highlighted in red, as activators (positive log fold change) and repressors (negative log fold change). N-terminal screen identified 9 activators and 211 repressors, C-terminal screen identified 5 activators and 208 repressors.

FIGS. 11A and 11B show transcriptional activation of frataxin in WT-iPSCs facilitated by dSaCas9 transcription activator N-terminal (FIG. 11A) and C-terminal (FIG. 11B) fusion proteins identified in the screen. WT-iPSCs stably expressing frataxin promoter-targeting gRNA A were transduced with dSaCas9 fusion proteins comprising indicated effectors, including positive control (2× VP64) and negative control peptides. Expression was assessed by RT-qPCR in comparison to negative control.

FIGS. 12A and 12B show transcriptional activation of frataxin in FA-iPSCs harboring long trinucleotide repeats facilitated by dSaCas9 transcription activator N-terminal (FIG. 12A) and C-terminal (FIG. 12B) fusion proteins identified in the screen. FA-iPSCs stably expressing frataxin promoter-targeting gRNA A were transduced with dSaCas9 fusion proteins comprising indicated effectors, including positive control (2× VP64) and negative control peptides. Expression was assessed by RT-qPCR in comparison to negative control.

FIGS. 13A and 13B show frataxin mRNA expression (FIG. 13A) as assessed by qRT-PCR and frataxin protein expression (FIG. 13B) as assessed by ELISA, in cardiomyocytes derived from FA-iPSCs or WT-iPSCs for the indicated conditions. FA-iPSC-derived cardiomyocytes were transduced with dSaCas9-2× VP64 and indicated FXN-targeting gRNA(s). Negative control cells were transduced with dSaCas9-2× VP64 and a non-targeting gRNA (NT gRNA), or with a puromycin resistance cassette alone (puro control (FA)). Expression is assessed relative to WT control cells (puro control (WT)).

FIG. 14 shows frataxin mRNA expression as assessed by qRT-PCR in neurons derived from FA-iPSCs or WT-iPSCs for the indicated conditions. FA-iPSC-derived neurons were transduced with dSaCas9-2× VP64 and indicated FXN-targeting gRNA(s). Negative control cells were transduced with dSaCas9-2× VP64 and a non-targeting gRNA (NT gRNA), or with a puromycin resistance cassette alone (puro control (FA)). Expression is assessed relative to WT control cells (puro control (WT)).

FIG. 15 shows frataxin mRNA expression as assessed by qRT-PCR in non-human primate fibroblasts from Crab-eating monkey (Macaca fascicularis) and Rhesus monkey (Macaca mulatta) for the indicated conditions. Cells were transduced with dSaCas9-2× VP64 and indicated FXN-targeting gRNA(s). Negative control cells were transduced with dSaCas9-2× VP64 and a non-targeting gRNA (NT gRNA), or with a puromycin resistance cassette alone (puro control).

FIGS. 16A and 16B show frataxin protein expression as assessed by ELISA in the liver of a humanized FA mouse model or healthy control, treated with AAV9 vectors encoding dSaCas9-2× VP64 and indicated FXN-targeting gRNA(s). As controls, FA mouse model or healthy control mice were treated with AAV9 vectors encoding dSaCas9-2× VP64 and a non-targeting gRNA. FIG. 16A indicates FXN protein pg per μg of loaded protein into the assay [pg/μg protein], and FIG. 16B indicates FXN protein levels normalized to the average FXN protein levels from healthy control mice.

FIGS. 17A and 17B show frataxin protein expression as assessed by ELISA in the heart of a humanized FA mouse model or healthy control, treated with AAV9 vectors encoding dSaCas9-2× VP64 and indicated FXN-targeting gRNA(s). As controls, FA mouse model or healthy control mice were treated with AAV9 vectors encoding dSaCas9-2× VP64 and a non-targeting gRNA. FIG. 17A indicates FXN protein pg per μg of loaded protein into the assay [pg/μg protein], and FIG. 17B indicates FXN protein levels normalized to the average FXN protein levels from healthy control mice.

FIGS. 18A and 18B show succinate dehydrogenase (SDH) enzymatic activity in the heart of a humanized FA mouse model or healthy control, treated with AAV9 vectors encoding dSaCas9-2× VP64 and indicated FXN-targeting gRNA(s). As controls, FA mouse model or healthy control mice were treated with AAV9 vectors encoding dSaCas9-2× VP64 and a non-targeting gRNA. FIG. 18A indicates mU of SDH activity per mg loaded protein into the assay [mu/mg], and FIG. 18B indicates SDH activity normalized to the SDH activity from healthy control mice.

FIGS. 19A and 19B show AAV vector genome quantification as assessed by ddPCR, in the liver (FIG. 19A) and the heart (FIG. 19B) of the humanized FA mouse model or healthy control, treated with AAV9 vectors encoding dSaCas9-2× VP64 and indicated FXN-targeting gRNA(s). As controls, FA mouse model or healthy control mice were treated with AAV9 vectors encoding dSaCas9-2× VP64 and a non-targeting gRNA.

### DETAILED DESCRIPTION

Provided herein DNA-targeting systems that bind to or target a frataxin (FXN) locus. In some aspects, the DNA-targeting systems include fusion proteins. In some aspects, the DNA-targeting systems include guide RNAs (gRNAs). In some aspects, the DNA-targeting systems include fusion proteins and gRNAs. Provided herein are compositions, such as DNA-targeting systems, including fusion proteins, gRNAs, and pluralities and combinations thereof, that bind to or target a FXN locus. Also provided are fusion proteins that bind to or target FXN. Also provided are gRNAs that bind to or target FXN. In some aspects, the provided DNA-targeting systems, including fusion proteins, gRNAs, bind to, target, and/or modulate the expression of FXN. Also provided are polynucleotides, vectors, cells, and pluralities and combinations thereof, that encode or comprise the DNA-targeting systems, fusion proteins, gRNAs or components thereof.

Also provided are methods and uses related to any of the provided compositions and combinations, for example, in modulating the expression of FXN, and/or in the treatment of diseases or disorders associated with reduced activity, mutation and/or dysregulation of expression of FXN, such as Friedreich's ataxia (FA). In some aspects, also provided are methods and uses related to any of the provided compositions and combinations, for example, in modulating the expression of FXN, and/or in the treatment or therapy of diseases or disorders associated with the activity, function or expression, for example dysregulation or reduced activity, function or expression of FXN, such as FA.

In some aspects, the provided embodiments are based on an observation described herein that the level of a human FXN locus expression in cells from FA patients, including in induced pluripotent stem cells (iPSCs) generated from FA patient cells, can be increased or restored using an exemplary DNA-targeting system comprising a deactivated Cas9 (dCas9)-transcriptional activator fusion protein and a gRNA targeting a promoter region or an enhancer region of a human FXN locus. In addition, the results described herein also show that combinations of two or more DNA-targeting systems targeting different target sites of a human FXN locus can result in a synergistic increase in expression of a human FXN locus. The provided embodiments are also based on an observation that certain fusion proteins comprising a DNA-targeting domain and an effector domain is identified based on screening a library of effector domains for their effect in increased expression of a human FXN locus. The embodiments described herein demonstrate consistent and effective increase or restoration of FXN expression, in cells from patients with FA, at both the mRNA and protein level, supporting the utility of the approaches in treating FA or other diseases or disorders that are associated with reduced activity, mutation and/or dysregulation of expression of FXN.

Certain genetic development disorders, including FA, are associated with reduced activity, mutation and/or dysregulation of expression of a frataxin (FXN) gene. FA is an autosomal recessive neurodegenerative and cardiac disease, is caused by a trinucleotide repeat expansion mutation in the FXN locus. FA can result in ataxia, areflexia, loss of vibratory sense and proprioception, dysarthria, cardiomyopathy and/or associated arrhythmias, among other symptoms. Existing treatment of FA is directed towards symptoms and providing support. Treatments that address the fundamental etiology and disease mechanism are needed. Provided are embodiments, including DNA-targeting systems, fusion proteins, guide RNAs (gRNAs), polynucleotides, vectors, cells, kits, and pluralities and combinations thereof, and methods and uses thereof, that meet such needs.

In some aspects, the provided embodiments offer an advantage of targeting regulatory DNA elements of a frataxin locus within a particular genomic region, such as an enhancer region, for modulating transcription. In some aspects, the provided embodiments offer an advantage of facilitating controlled, additive and/or synergistic activation of FXN by targeting two or more sites within regulatory DNA elements of FXN. In some aspects, the provided embodiments offer an advantage of increasing FXN expression to a level that is therapeutically relevant for subjects having a disease or disorder that involve the activity, function or expression of FXN, such as FA. In some aspects, the provided embodiments also offer an advantage of providing various effector domains which are capable of inducing transcription activation, for example, at a particular target locus such as FXN.

In certain aspects, the provided embodiments offer the ability to fine tune and tightly regulate the level of expression and/or activity of frataxin in a cell or a subject. As described further below, the control of the expression and/or activity of frataxin at a particular level or within an optimal window is critical for the survival and normal function of the subject, as the reduction of expression can result in diseases or disorders such as Friedreich's Ataxia (FA) and in some cases, substantial overexpression can result in toxicity, organ dysfunction and reduction of life span. Accordingly, the level of expression and/or activity of frataxin must be fine-tuned to be within an optimal window. Even for the treatment of diseases or disorders associated with reduced expression and/or activity of frataxin, such as in FA, there is a need to tightly regulate the expression and/or activity of frataxin and avoid overexpression. The provided embodiments permit such fine tuning of expression of FXN without the need for introducing additional copies of FXN into the cell, which could result in toxic overexpression of FXN.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Compositions and Methods for Modulating Expression of Frataxin (FXN)

Provided herein are compositions such a DNA-targeting systems that bind to or target a frataxin (FXN) locus. In some aspects, the provided DNA-targeting systems include fusion proteins and/or guide RNAs (gRNAs). In some aspects, provided are polynucleotides, vectors that encode any of the DNA-targeting systems, fusion proteins and/or components of kits. In some embodiments, provided are cells, kits, systems and pluralities and combinations thereof, that comprise any of the DNA-targeting systems, fusion proteins or gRNAs described herein.

Provided herein are DNA-targeting systems comprising a DNA-targeting domain that binds to a target site in a regulatory DNA element of a frataxin (FXN) locus. In some of any of the embodiments provided herein, binding of the DNA-targeting domain to the target site does not introduce a genetic disruption or a DNA break at or near the target site. In some aspects, the provided DNA-targeting systems comprise a fusion protein comprising a DNA-targeting domain and an effector domain, and binds to a target site in a regulatory DNA element of a FXN locus. In some aspects, the DNA-targeting system also comprises a guide RNA (gRNA). In some aspects, when administered to a subject or delivered or introduced into a cell that exhibits dysregulation or reduced activity, function or expression of FXN, the provided DNA-targeting systems can lead to an increase of or a restoration of, the activity, function or expression of FXN. In some aspects, also provided are methods and uses related to any of the provided compositions, for example, in modulating the expression of FXN, and/or in the treatment or therapy of diseases or disorders associated with dysregulation or reduced activity, function or expression of FXN, such as FA

In some embodiments, the DNA-targeting systems are targeted to one or more target sites located within a regulatory DNA element of a FXN locus, such as a promoter or an enhancer. In some embodiments, the DNA-targeting systems are targeted to at least 2, 3, 4, 5, 6, 7, 8, 9 or 10 target sites within a regulatory DNA element of a FXN locus. In some embodiments, the DNA-targeting systems are targeted to one or more target sites located within a promoter of a FXN locus, and one or more target sites located within an enhancer of a FXN locus.

In some embodiments, the DNA-targeting system comprises a DNA-targeting domain comprising a Clustered Regularly Interspaced Short Palindromic Repeats associated (Cas)-guide RNA (gRNA) combination comprising (a) a Cas protein or a variant thereof and (b) at least one gRNA; a zinc finger protein (ZFP); a transcription activator-like effector (TALE); a meganuclease; a homing endonuclease; or an I-SceI enzyme or a variant thereof. In some aspects, the DNA-targeting domain comprises a catalytically inactive variant of any of the foregoing. In some embodiments, the DNA-targeting system comprises a DNA-targeting domain comprising a Cas-gRNA combination comprising (a) a Cas protein or a variant thereof, and (b) at least one gRNA. In some embodiments, the at least one gRNA comprises at least 2, 3, 4, 5, 6, 7, 8, 9 or 10 gRNAs. In some embodiments, the gRNAs are targeted to one or more target sites located within a regulatory DNA element of a FXN locus, such as a promoter or an enhancer. In some embodiments, the gRNAs are targeted to one or more target sites located within a promoter of a FXN locus, and one or more target sites located within an enhancer of a FXN locus.

In some aspects, the provided embodiments involve modulating transcription of an endogenous FXN locus in a cell. In some aspects, the provided embodiments involve increasing transcription of an endogenous FXN locus in a cell. In some embodiments, the cell, such as the cell to be treated with the provided embodiments, has a GAA trinucleotide repeat expansion in the FXN locus. In some embodiments, the cell, such as the cell to be treated with the provided embodiments, is from or in a subject with Friedreich's ataxia. In some embodiments, the cell, such as the cell to be treated with the provided embodiments, exhibits reduced expression of FXN compared to a cell from a subject without Friedreich's ataxia.

In some aspects, in a cell introduced with or contacted with any of the DNA-targeting systems, gRNA, combinations, fusion proteins, polynucleotides, plurality of polynucleotides, vectors, plurality of vectors or components or portions thereof provided herein, the expression of FXN is increased at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold, compared to a cell that has not been introduced or contacted. In some embodiments, the expression is increased by less than about 10-fold, 9-fold, 8-fold, 7-fold or 6-fold.

In some embodiments, the subject is a human. In some embodiments, the cell is a heart cell, a skeletal muscle cell, a nervous system cell, or an induced pluripotent stem cell. In some embodiments, the introducing, contacting or administering is carried out in vivo or ex vivo.

### A. FXN and Friedreich's Ataxia (FA)

Frataxin is a protein (exemplary amino acid sequences of human Frataxin Isoform 1: NCBI NM_000144.4, NP_000135 (210 aa), SEQ ID NO: 1; exemplary amino acid sequences of human Frataxin Isoform 2: NM_181425, NP_852090 (196 aa), SEQ ID NO:2; exemplary amino acid sequences of human Frataxin Isoform 3: NM_001161706, NP_001155178, (171 aa), Uniprot Q16595; ENTREZ 2395; Ensembl ENSG00000165060; OMIM: 606829) found in cells throughout the body, with the highest levels in tissues with a high metabolic rate including heart, neurons, spinal cord, liver, pancreas (Langerhans cells), and muscles used for voluntary movement (skeletal muscles). Within cells, frataxin is mainly found in mitochondria. In some aspects, frataxin promotes the biosynthesis of heme as well as the assembly and repair of iron-sulfur clusters by delivering Fe2⁺ to proteins involved in these pathways. In some aspects, frataxin also plays a primary role in the protection against oxidative stress through its ability to catalyze the oxidation of Fe2⁺ to Fe3⁺ and to store large amounts of the metal in the form of a ferrihydrite mineral. In some aspects, frataxin is processed in two steps by mitochondrial processing peptidase (MPP). MPP first cleaves the precursor to intermediate form and subsequently converts the intermediate to a mature protein. Thus, in cells, 3 forms exist. For isoform 1, these forms are frataxin (56-210); frataxin (78-210) and frataxin (81-210), which is the main form of mature frataxin.

Friedreich's ataxia (FA) is a monogenic autosomal recessive neurodegenerative and cardiac disease, associated with trinucleotide repeat expansion mutations in the first intron of the frataxin gene (FXN), which is located on the long arm of chromosome 9. The vast majority of FA patients are homozygous for guanine-adenine-adenine (GAA) trinucleotide repeat expansions in the first intron of FXN. FA is a degenerative neuromuscular disorder, and can result in ataxia, areflexia, loss of vibratory sense and proprioception, dysarthria, cardiomyopathy and/or associated arrhythmias, among other symptoms, and death. The mutation leads to reduced expression of the frataxin mRNA and protein. Frataxin is essential for proper functioning of mitochondria. As noted above, Frataxin is involved in the removal of iron and when Frataxin is reduced, iron builds up and causes free radical damage. Nerve and muscle cells are particularly sensitive to these deleterious effects. FA occurs in approximately 1 in 50,000 persons in European populations but is much more frequent in the province of Quebec in Canada, because of founder effects. Males and females are affected equally. In the classic form, FA symptoms appear during or before the second decade of life. FA is characterized by ataxia, areflexia, loss of vibratory sense and proprioception and dysarthria. Moreover, FA patients often have systemic involvement, with cardiomyopathy, diabetes mellitus and scoliosis. Early death can result from cardiomyopathy or associated arrhythmias. Degeneration of the dorsal root ganglion cells, their ascending dorsal spinal columns and the spinocerebellar tracts results in a progressive sensory ataxia. Many patients are wheelchair bound by their third decade of life. Associated oculomotor problems include optic atrophy, square-wave jerks and difficulty with fixation. Importantly, cognitive abilities are relatively spared. However, many patients suffer from depression.

The class of mutation most commonly associated with FA is unstable hyper-expansion of a GAA trinucleotide repeat located in the first intron of the frataxin gene. In normal subjects, there are approximately 6-34 repeats, whereas expansions associated with FA are typically 150 or more repeats, and may vary from 44 to 1700 repeats, with most abnormal alleles ranging from 600 to 900 repeats. Increased trinucleotide repeat expansion lengths are associated with decreased FXN levels, earlier onset of disease, and increased disease severity. Patients with fewer repeats (150-200) have milder symptoms than those with longer sections with more triplet repeats (350 to 650). In some severely affected patients there are up to 1700 repeats. Since frataxin expansion mutations are located in an intron, the amino acid sequence of the frataxin protein is not altered. However, approximately 1-3% of FA patients are compound heterozygotes with an expansion on one allele and a conventional mutation (e.g. a missense, nonsense, or deletion mutation) on the other. Some patients with a missense mutation can have less severe symptoms because the mutated protein in still functional. FA symptoms are not observed in heterozygous carriers.

In some aspects, the term “trinucleotide repeat expansion” means a series of three bases (for example, GAA) repeated at least twice. In certain examples, the trinucleotide repeat expansion may be located in intron 1 of a FXN locus, gene or nucleic acid. In certain examples, a pathogenic trinucleotide repeat expansion includes at least 66 or 70 repeats of GAA in a FXN nucleic acid and is associated with disease. In other examples, a pathogenic trinucleotide repeat expansion includes at least 67, 68, 69, 70, 75, 80, 85, 90, 95, 100, 150, 200, 250, 300, 400, 500, 800, 1000 or more repeats. In certain examples, the repeats are consecutive. In certain examples, the repeats are interrupted by one or more nucleobases. In certain examples, a wild-type trinucleotide repeat expansion includes 12 or fewer repeats of GAA in a FXN nucleic acid. In other examples, a wild-type trinucleotide repeat expansion includes 11, 10, 9, 8, 7, 6, 5, 4, 3, 2, or 1 repeat.

FXN has been associated with diseases and disorders such as, but not limited to, Alzheimer's Disease, Amyotrophic Lateral Sclerosis, Apraxias, Ataxia, Ataxia Telangiectasia, Hereditary Ataxias, Bloom Syndrome, Brain Neoplasms, Malignant tumor of colon, Dilated Cardiomyopathy, Hypertrophic Cardiomyopathy, Cerebellar Ataxia, Cystic Fibrosis, Diabetes, Diabetes Mellitus, Non-Insulin-Dependent Diabetes Mellitus, Dysarthria, Dystonia, Fragile X Syndrome, Friedreich's Ataxia, Heart Diseases, Cardiomegaly, Hemochromatosis, Herpes Simplex Infections, Huntington Disease, Liver neoplasms, Machado-Joseph Disease, Metabolic Diseases, Myocardial Infarction, Myotonic Dystrophy, nervous system disorder, Neuroblastoma, Neuromuscular Diseases, Pallor, Parkinson Disease, Peripheral Neuropathy, Protein Deficiency, Restless Legs Syndrome, Schizophrenia, unspecified Scoliosis, Hereditary Spastic Paraplegia, Spinocerebellar Ataxia, Left Ventricular Hypertrophy, Sensory neuropathy, Tumor Progression, Neurologic Symptoms, Paroxysmal atrial fibrillation, Hypoalbuminemia, Impaired glucose tolerance, Iron Overload, Adenocarcinoma of colon, Depletion of mitochondrial DNA, Ventricular septal hypertrophy, Malignant neoplasm of prostate, Hereditary hemochromatosis, Dystonia Disorders, Congenital Myotonic Dystrophy, Spastic, Neurodegenerative Disorders, Congenital scoliosis, Colon Carcinoma, Central neuroblastoma, Acquired scoliosis, cardiac symptom, Appendicular Ataxia, Mitochondrial Diseases, Heredodegenerative Nervous System Disorders, Spinocerebellar Ataxia Type 1, Cardiomyopathies, Ceruloplasmin deficiency, Hypertrophic Cardiomyopathy Familial, Degenerative disorder, Head titubation, Non-Neoplastic Disorder, X-Linked Bulbo-Spinal Atrophy, Fragile X Tremor/Ataxia Syndrome, Friedreich's Ataxia With Retained Reflexes, Ataxia With Vitamin E Deficiency, Spinocerebellar Ataxia Autosomal Recessive 1, Friedreich's Ataxia 1, Hereditary Neurodegenerative Disorder, and Spastic Paraplegia Type 7. Editing the FXN gene using any of the methods described herein may be used to treat, prevent and/or mitigate the symptoms of the diseases and disorders described herein.

Frataxin is a mitochondrial iron chaperone suggested to be important for iron-sulfur processing. This protein is found throughout the human body, but is enriched in the heart, spinal cord, liver, pancreas and muscles. Expansions of a trinucleotide GAA repeat region in the FXN gene, and subsequent frataxin protein deficiency, causes Friedreich's Ataxia. The GAA repeat region is located in the middle of an Alu element in the first intron of the FXN gene. In most people, the number of GAA repeats in the FXN gene is fewer than 12. Individuals with 12-33 uninterrupted GAA repeats are said to be asymptomatic. However, as these repeats are unstable and more likely to expand during meiosis, such individuals are at increased risk of having affected children. In people with Friedreich's Ataxia, the GAA segment is abnormally repeated from 66 to over 1,000 times. The number of repeats in the gene correlates with the age of onset and severity of the disease. Individuals with fewer than 300 GAA repeats tend to have later symptom onset (after age 25) than those with larger GAA trinucleotide repeats (e.g. 600 to 900 repeats). The abnormal repeat expansion can result in an RNA processing defect, which leads to dysregulation of translation and reduced amount of the FXN protein in cells.

FA is usually diagnosed in the first or second decade and affects 1 in 50,000 people in the United States. Friedreich's Ataxia is a progressive movement disorder characterized by loss of strength and sensation, muscle stiffness and impaired speech. Individuals with Friedreich's Ataxia may also have cardiomyopathy, diabetes, vision or hearing loss, and/or scoliosis. Currently there is no treatment for Friedreich's Ataxia, only symptom management.

The FXN gene is also associated with a number of other disorders, including hereditary ataxia, X-linked sideroblastic anemia with ataxia, tabes dorsalis, spinocerebellar degeneration, tertiary neurosyphilis, a transferrinemia, spinocerebellar ataxia, scoliosis, hemochromatosis, fragile X syndrome, mitochondrial disorders and cardiomyopathy.

### B. Modulating Expression of FXN

In some aspects, provided are compositions, methods and related uses, that can be employed to modulate the expression of FXN, such as in a cell or a subject. In some aspects, the provided compositions, methods and uses can be employed to activate or increase the expression of FXN in the cell or the subject. In some aspects, the subject has or is suspected of having a disease or disorder associated with reduced activity, inactivation, mutation and/or dysregulation of expression of the FXN gene, such as FA. In some aspects, disease or disorders such as Friedreich's Ataxia (FA) is associated with reduced FXN expression levels compared to in a normal individual. In some aspects, by modulating, such as by activating or increasing the expression of FXN, the provided compositions, methods and uses can be employed to treat or ameliorate the disease or disorder associated with reduced activity, mutation and/or dysregulation of FXN.

In some aspects, by modulating, such as by activating or increasing the expression of FXN, the provided compositions, methods and uses can be employed to restore or recover the expression or activity of FXN in a subject or a cell with a disease or disorder associated with reduced activity, mutation and/or dysregulation of FXN, such that the expression or activity of FXN is increased at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold, compared to the expression or activity of FXN in the subject or cell with the disease or disorder in the absence of the provided compositions or uses. In some aspects, the expression or activity is increased by less than about 10-fold, 9-fold, 8-fold, 7-fold or 6-fold. In some aspects, by modulating, such as by activating or increasing the expression of FXN, the provided compositions, methods and uses can be employed to restore or recover the expression or activity of FXN in a subject or a cell with a disease or disorder associated with reduced activity, mutation and/or dysregulation of FXN, such that the expression or activity of FXN is increased to at least at or about 30%, 40%, 50%, 60%, 70%, 75%, 80%, 85%, 90%, 95%, 100%, 105%, 110%, 120%, 125%, 150%, 175%, 200%, 225%, 250%, 300%, 400%, or 500%, of the expression or activity of FXN in an individual or a cell without the disease or disorder or in a wild-type cell. Increasing the expression of FXN mRNA and/or protein, can lead to recovery or restoration of expression of the FXN gene and be used for treatment and/or prevention of such diseases or disorders.

In certain cases, overexpression of FXN can also lead to toxicity in cells and tissues, organ dysregulation and/or reduction of life span (see, e.g., Vannocci et al., Disease Models & Mechanisms (2018) 11, dmm032706; Belbellaa et al., Molecular Therapy—Methods & Clinical Development (2020) 19:120-138; Navarro et al., PLOS ONE 6(7): e21017). In some aspects, the provided embodiments modulate, such as by activate or increase the expression of FXN, however, the level of expression does not result in substantial overexpression of FXN to a level that would result in toxicity, organ dysregulation, cell death or reduction of life span. For example, the expression or activity of FXN is increased by less than about 10-fold, 9-fold, 8-fold, 7-fold or 6-fold, compared to the expression or activity of FXN in the subject or cell with the disease or disorder in the absence of the provided compositions or uses. In certain aspects, the expression or activity of FXN is increased to at least at or about 30%, 40%, 50%, 60%, 70%, 75%, 80%, 85%, 90%, 95%, 100%, 105%, 110%, 120%, 125%, 150%, 175%, 200%, 225%, 250%, 300%, 400%, or 500%, of the expression or activity of FXN in an individual or a cell without the disease or disorder or in a wild-type cell. In certain aspects, the expression or activity of FXN does not exceed about 500%, 600%, 700%, 800%, 900%, or 1000% of the expression or activity of FXN in an individual or a cell without the disease or disorder or in a wild-type cell. In some aspects, the provided embodiments offer the ability to fine tune and tightly regulate the expression level of FXN, for example, for treating diseases or disorders associated with reduced activity, inactivation, mutation and/or dysregulation of expression of the FXN gene, such as FA.

### II. DNA-Targeting Systems

Provided herein are DNA-targeting systems comprising a DNA-targeting domain that binds to a target site in a regulatory DNA element of a frataxin (FXN) locus. Exemplary components and features of the DNA-targeting systems are provided herein. In some aspects, the DNA-targeting system comprises one or more of any of the components described herein, such as one or more DNA-targeting domains, one or more fusion proteins, such as one or more fusion proteins comprising one or more DNA-targeting domains and one or more effector domains, one or more gRNAs, or any component, portion or fragment thereof, or any combination thereof.

In some aspects, the DNA-targeting system comprises a DNA-targeting domain and one or more guide RNAs (gRNAs). In some aspects, the DNA-targeting system comprises a fusion protein and one or more gRNAs. In some aspects, the DNA-targeting system comprises a DNA-targeting domain and a gRNA. In some aspects, the DNA-targeting system comprises a fusion protein. In some aspects, the DNA-targeting system comprises a fusion protein and a gRNA. In some aspects, the DNA-targeting system comprises a DNA-targeting domain.

In some embodiments, binding of the DNA-targeting domain to the target site does not introduce a genetic disruption or a DNA break at or near the target site.

In some embodiments, provided are DNA-targeting systems capable of specifically targeting a target site in a FXN gene or DNA regulatory element thereof, and increasing transcription of the FXN gene. In some embodiments, the DNA-targeting systems include a DNA-targeting domain that binds to a target site in the FXN gene or regulatory DNA element thereof. In provided embodiments, the DNA-targeting systems additionally include at least one effector domain that is able to epigenetically modify one or more DNA bases of the FXN gene or regulatory element thereof, in which the epigenetic modification results in an increase in transcription of the FXN gene (e.g. activates transcription or increases transcription of FXN compared to the absence of the DNA-targeting system). Hence, the terms DNA-targeting system and epigenetic-modifying DNA targeting system may be used herein interchangeably. In some embodiments, the DNA-targeting system includes a fusion protein comprising (a) a DNA-targeting domain capable of being targeted to the target site; and (b) at least one effector domain capable of increasing transcription of the FXN gene. For instance, the at least one effector domain is a transcription activation domain.

In some embodiments, the DNA-targeting domain comprises or is derived from a CRISPR associated (Cas) protein, zinc finger protein (ZFP), transcription activator-like effectors (TALE), meganuclease, homing endonuclease, I-SceI enzyme, or variants thereof. In some embodiments, the DNA-targeting domain comprises a catalytically inactive (e.g. nuclease-inactive or nuclease-inactivated) variant of any of the foregoing. In some embodiments, the DNA-targeting domain comprises a deactivated Cas9 (dCas9) protein or variant thereof that is a catalytically inactivated so that it is inactive for nuclease activity and is not able to cleave the DNA.

In some embodiments, the DNA-targeting domain comprises or is derived from a Cas protein or variant thereof, such as a nuclease-inactive Cas or dCas (e.g. dCas9, and the DNA-targeting system comprises one or more guide RNAs (gRNAs). In some embodiments, the gRNA comprises a spacer sequence that is capable of targeting and/or hybridizing to the target site. In some embodiments, the gRNA is capable of complexing with the Cas protein or variant thereof. In some aspects, the gRNA directs or recruits the Cas protein or variant thereof to the target site.

In some embodiments, the effector domain comprises a transcription activation domain, and/or is capable of increasing transcription of the gene. In some embodiments, the effector domain induces, catalyzes or leads to transcription activation, transcription co-activation, or transcription elongation. In some aspects, the effector domain is selected from VP64, p65, Rta, p300, CBP, VPR, VPH, Rta, p300, HSF1, a TET protein (e.g. TET1), an ERF protein (e.g. ERF1, ERF3), LSD1, SunTag, a partially or fully functional fragment or domain thereof, or a combination of any of the foregoing. In some embodiments, the effector domain may include a domain, portion, or variant of a protein selected from: DPOLA, ENL, FOXO3, HSH2D, NCOA2, NCOA3, PSA1, PYGO1, RBM39, HERC2, and NOTCH2. In some embodiments, the effector domain is VP64. In some embodiments, the fusion protein of the DNA-targeting system comprises dCas9-VP64.

In some embodiments, the DNA-targeting system comprises a DNA-targeting domain. In some embodiments the DNA-targeting domain comprises a DNA-binding protein or DNA-binding nucleic acid. In some embodiments, the DNA-targeting domain specifically binds to or hybridizes to a particular site or position in the genome, e.g., a target, target site, or target position. In some aspects, the DNA-targeting domain is coupled to, fused to or complexed with an effector domain, such as any effector domain described herein, for example, in Section II.D.

In some embodiments, the DNA-targeting system comprises various components, such as an RNA-guided nuclease, variant thereof, or fusion protein comprising the RNA-guided nuclease or variant thereof, or a fusion protein comprising a DNA-targeting domain and an effector domain. In some embodiments, the DNA-targeting system comprises a DNA-targeting molecule that comprises a DNA-binding protein such as one or more zinc finger protein (ZFP) or transcription activator-like effectors (TALEs), fused to an effector domain.

In some embodiments, the DNA-targeting system specifically targets at least one target site in a regulatory DNA element of a frataxin (FXN) locus. In some embodiments, the DNA-targeting system comprises a ZFP, TALE or a CRISPR/Cas9 combination that specifically binds to, recognizes, or hybridizes to the target site(s). In some embodiments, the CRISPR/Cas9 system includes an engineered crRNA/tracr RNA (i.e. “single guide RNA”). In some embodiments, the DNA-targeting system comprises nucleases or variants thereof based on the Argonaute system (e.g., from T. thermophilus, known as ‘TtAgo’ (Swarts et al., (2014) Nature 507(7491): 258-261).

In some embodiments, the DNA-targeting domain comprises a Clustered Regularly Interspaced Short Palindromic Repeats associated (Cas)-guide RNA (gRNA) combination that includes (a) a Cas protein or a variant thereof and (b) at least one gRNA; a zinc finger protein (ZFP); a transcription activator-like effector (TALE); a meganuclease; a homing endonuclease; or a I-SceI enzyme or a variant thereof. In some embodiments, the DNA-targeting domain comprises a catalytically inactive variant of any of the foregoing. In some embodiments, the DNA-targeting domain comprises a Cas-gRNA combination that includes (a) a Cas protein or a variant thereof and (b) at least one gRNA. In some embodiments, the variant Cas protein lacks nuclease activity or is a deactivated Cas (dCas) protein.

Also provided herein are DNA-targeting systems comprising a DNA-targeting domain, that binds to a target site in a regulatory DNA element of a frataxin (FXN) locus and comprises a Cas-guide RNA (gRNA) combination comprising: (a) a variant Cas protein that lacks nuclease activity or that is a deactivated Cas (dCas) protein; and (b) at least one gRNA comprising at least one gRNA spacer sequence that is capable of hybridizing to the target site or is complementary to the target site.

Also provided herein are DNA-targeting systems comprising a DNA-targeting domain that comprises a Cas-guide RNA (gRNA) combination comprising: (a) a Staphylococcus aureus deactivated Cas9 protein (dSaCas9) protein set forth in SEQ ID NO:72 fused to at least one effector domain that induces transcription activation; and (b) at least one gRNA comprising the gRNA spacer sequence set forth in SEQ ID NO:42.

Also provided herein are DNA-targeting systems comprising a DNA-targeting domain that comprises a Cas-guide RNA (gRNA) combination comprising: (a) a Staphylococcus aureus deactivated Cas9 protein (dSaCas9) protein set forth in SEQ ID NO:72 fused to at least one effector domain that induces transcription activation; and (b) at least one gRNA comprising the gRNA spacer sequence set forth in SEQ ID NO:22.

Also provided herein are DNA-targeting systems comprising a DNA-targeting domain that comprises a Cas-guide RNA (gRNA) combination comprising: (a) a Staphylococcus aureus deactivated Cas9 protein (dSaCas9) protein set forth in SEQ ID NO:72 fused to at least one effector domain that induces transcription activation; and (b) at least one gRNA comprising the gRNA spacer sequence set forth in SEQ ID NO:28.

### A. Target Site at the FXN Locus

In some aspects, provided are compositions, methods and uses, such as DNA-targeting systems, DNA-targeting domains, components of the DNA-targeting domains, such as at least one gRNA, fusion proteins, and pluralities and combinations thereof, polynucleotides, vectors, cells, and pluralities and combinations thereof, that encode or comprise the DNA-targeting systems, fusion proteins, gRNAs, or pluralities or combinations thereof, that can target one or more particular genomic locations related to the FXN locus, such as a regulatory DNA element of the FXN locus.

In some embodiments, the target site is in a cell, such as any suitable cell. In some embodiments, the cell is in or from any suitable organism, such as a human, mouse, dog, horse, rabbit, cattle, pig, hamster, gerbil, mouse, ferret, rat, cat, non-human primate, monkey, etc. In some embodiments, the cell is in or from a human. In some embodiments, the cell is any suitable cell, such as an immune cell (e.g. a T cell, B cell, or antigen-presenting cell), a liver cell (e.g. a hepatocyte), a cell of a nervous system (e.g. a neuron or glial cell), a heart cell (e.g. a cardiomyocyte) or a stem cell (e.g. an embryonic stem cell or induced pluripotent stem cell).

In some embodiments, the target site is located in a regulatory DNA element of a frataxin (FXN) locus. In some embodiments, the target site is located within a promoter, upstream regulatory element (e.g., enhancer), exon, intron, 5′ untranslated region (UTR), 3′ UTR, or downstream regulatory element.

In some embodiments, the target site is located within a FXN locus. In some embodiments the target site is located within a regulatory DNA element (e.g. a cis-, trans-, distal, proximal, upstream, or downstream regulatory DNA element) of a FXN locus. In some embodiments, the target site is located within a promoter, enhancer, exon, intron, untranslated region (UTR), 5′ UTR or 3′ UTR. In some embodiments the target site is located within a sequence and/or sequences of unknown or known function that are suspected of being able to control expression of FXN.

In some embodiments one or more target sites, such as one or more target sites located within a regulatory DNA element (e.g. a cis-, trans-, distal, proximal, upstream, or downstream regulatory DNA element) of a FXN locus. In some embodiments, the target site is located within a promoter, enhancer, exon, intron, untranslated region (UTR), 5′ UTR or 3′ UTR are targeted.

In some aspects, an exemplary frataxin (FXN) transcript is set forth in RefSeq NM_000144) (transcript variant 1); Gencode Transcript: ENST00000484259.3; Gencode Gene: ENSG00000165060.15. Genomic coordinates for an exemplary transcript (including UTRs) for FXN include hg38 chr9:69,035,752-69,079,076 (Size: 43,325 Total Exon Count: 5 Strand: +). Genomic coordinates for the coding region for this transcript variant include hg38 chr9:69,035,783-69,072,762 (Size: 36,980 Coding Exon Count: 5).

In some aspects, an exemplary frataxin (FXN) transcript is set forth in RefSeq NM_181425) (transcript variant 2); Gencode Transcript: ENST00000396366.6; Gencode Gene: ENSG00000165060.15. Genomic coordinates for an exemplary transcript (including UTRs) for FXN include hg38 chr9:69,035,762-69,073,022 (Size: 37,261 Total Exon Count: 5 Strand: +). Genomic coordinates for the coding region for this transcript variant include hg38 chr9:69,035,783-69,072,712 (Size: 36,930 Coding Exon Count: 5).

In some embodiments, the regulatory DNA element is located in a genomic region comprising the FXN locus.

In some embodiments, the target site is located within the genomic coordinates hg38 chr9:68,940,179-69,205,519. In some embodiments, the target site is selected from the sequence set forth in any one of SEQ ID NOS: 1-21, a contiguous portion thereof of at least 14 nt, or a complementary sequence of any of the foregoing.

In some embodiments, the regulatory DNA element is an enhancer.

In some embodiments, the target site is located within the genomic coordinates human genome assembly GRCh38 (hg38) chr9:69,027,282-69,028,497. In some embodiments, the target site is located within the genomic coordinates hg38 chr9:69,027,615-69,028,101. In some embodiments, the target site comprises the sequence set forth in SEQ ID NO:21, a contiguous portion thereof of at least 14 nt, or a complementary sequence of any of the foregoing. In some embodiments, the target site comprises a sequence set forth in any one of SEQ ID NOS:229-243, a contiguous portion thereof of at least 14 nt, or a complementary sequence of any of the foregoing. In some embodiments, the target site comprises a sequence set forth in any one of SEQ ID NOS:256-265, a contiguous portion thereof of at least 14 nt, or a complementary sequence of any of the foregoing.

In some embodiments, the target site is located within the genomic coordinates hg38 chr9:69,044,201-69,045,347. In some embodiments, the target site is located within the genomic coordinates hg38 chr9:69,030,752-69,031,507. In some embodiments, the target site is located within the genomic coordinates hg38 chr9:68,999,262-69,000,023. In some embodiments, the target site is located within the genomic coordinates hg38 chr9:69,085,468-69,086,426. In some embodiments, the target site is located within the genomic coordinates hg38 chr9:69,096,701-69,097,567. In some embodiments, the target site is located within the genomic coordinates hg38 chr9:69,120,690-69,123,549. In some embodiments, the target site is located within the genomic coordinates hg38 chr9:69,130,392-69,132,484.

In some embodiments, the DNA-targeting domain comprises a Cas-gRNA combination comprising (a) a Cas protein or a variant thereof and (b) at least one gRNA; and the gRNA comprises at least one gRNA spacer sequence comprising the sequence set forth in SEQ ID NO:42, or a contiguous portion thereof of at least 14 nt. In some embodiments, the gRNA further comprises the sequence set forth in SEQ ID NO:44. In some embodiments, the gRNA comprises the sequence set forth in SEQ ID NO:67.

In some embodiments, the regulatory DNA element is a promoter.

In some embodiments, the target site is located within the genomic coordinates hg38 chr9:69,034,622-69,036,670. In some embodiments, the target site is located within the genomic coordinates hg38 chr9:69,035,300-69,035,900. In some embodiments, the target site is located within the genomic coordinates hg38 chr9:69,034,900-69,035,900. In some embodiments, the target site comprises a sequence selected from any one of SEQ ID NOS: 1-10, a contiguous portion thereof of at least 14 nt, or a complementary sequence of any of the foregoing. In some embodiments, the target site comprises a sequence selected from any one of SEQ ID NOS:11-20, a contiguous portion thereof of at least 14 nt, or a complementary sequence of any of the foregoing. In some embodiments, the target site comprises a sequence selected from any one of SEQ ID NOS:244-255, a contiguous portion thereof of at least 14 nt, or a complementary sequence of any of the foregoing.

In some embodiments, the target site is at, near, or within a FXN locus. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a portion of the target site sequence described herein. In some aspects, the target site is a sequence having at least 80% sequence identity to all or a portion of the target site sequence described herein. In some aspects, the target site is a sequence having at least 85% sequence identity to all or a portion of the target site sequence described herein. In some aspects, the target site is a sequence having at least 90% sequence identity to all or a portion of the target site sequence described herein. In some aspects, the target site is a sequence having at least 91% sequence identity to all or a portion of the target site sequence described herein. In some aspects, the target site is a sequence having at least 92% sequence identity to all or a portion of the target site sequence described herein. In some aspects, the target site is a sequence having at least 93% sequence identity to all or a portion of the target site sequence described herein. In some aspects, the target site is a sequence having at least 94% sequence identity to all or a portion of the target site sequence described herein. In some aspects, the target site is a sequence having at least 95% sequence identity to all or a portion of the target site sequence described herein. In some aspects, the target site is a sequence having at least 96% sequence identity to all or a portion of the target site sequence described herein. In some aspects, the target site is a sequence having at least 97% sequence identity to all or a portion of the target site sequence described herein. In some aspects, the target site is a sequence having at least 98% sequence identity to all or a portion of the target site sequence described herein. In some aspects, the target site is a sequence having at least 99% sequence identity to all or a portion of the target site sequence described herein. In some aspects, the target site is a sequence having at least 99.5% sequence identity to all or a portion of the target site sequence described herein. In some aspects, the target site is a sequence having at least 99.9% sequence identity to all or a portion of the target site sequence described herein. In some aspects, the target site is a sequence having 100% sequence identity to all or a portion of the target site sequence described herein.

In some embodiments, the target site is selected from the sequence set forth in any one of SEQ ID NOS: 1-21, a contiguous portion thereof of at least 14 nt, or a complementary sequence of any of the foregoing.

In some embodiments, the target site comprises SEQ ID NO:1, a contiguous portion thereof of at least 14 nt, or a complementary sequence of thereof. In some embodiments, the target site comprises SEQ ID NO:2, a contiguous portion thereof of at least 14 nt, or a complementary sequence of thereof. In some embodiments, the target site comprises SEQ ID NO:3, a contiguous portion thereof of at least 14 nt, or a complementary sequence of thereof. In some embodiments, the target site comprises SEQ ID NO:4, a contiguous portion thereof of at least 14 nt, or a complementary sequence of thereof. In some embodiments, the target site comprises SEQ ID NO:5, a contiguous portion thereof of at least 14 nt, or a complementary sequence of thereof. In some embodiments, the target site comprises SEQ ID NO:6, a contiguous portion thereof of at least 14 nt, or a complementary sequence of thereof. In some embodiments, the target site comprises SEQ ID NO:7, a contiguous portion thereof of at least 14 nt, or a complementary sequence of thereof. In some embodiments, the target site comprises SEQ ID NO:8, a contiguous portion thereof of at least 14 nt, or a complementary sequence of thereof. In some embodiments, the target site comprises SEQ ID NO:9, a contiguous portion thereof of at least 14 nt, or a complementary sequence of thereof. In some embodiments, the target site comprises SEQ ID NO: 10, a contiguous portion thereof of at least 14 nt, or a complementary sequence of thereof. In some embodiments, the target site comprises SEQ ID NO:11, a contiguous portion thereof of at least 14 nt, or a complementary sequence of thereof. In some embodiments, the target site comprises SEQ ID NO: 12, a contiguous portion thereof of at least 14 nt, or a complementary sequence of thereof. In some embodiments, the target site comprises SEQ ID NO: 13, a contiguous portion thereof of at least 14 nt, or a complementary sequence of thereof. In some embodiments, the target site comprises SEQ ID NO:14, a contiguous portion thereof of at least 14 nt, or a complementary sequence of thereof. In some embodiments, the target site comprises SEQ ID NO: 15, a contiguous portion thereof of at least 14 nt, or a complementary sequence of thereof. In some embodiments, the target site comprises SEQ ID NO: 16, a contiguous portion thereof of at least 14 nt, or a complementary sequence of thereof. In some embodiments, the target site comprises SEQ ID NO: 17, a contiguous portion thereof of at least 14 nt, or a complementary sequence of thereof. In some embodiments, the target site comprises SEQ ID NO: 18, a contiguous portion thereof of at least 14 nt, or a complementary sequence of thereof. In some embodiments, the target site comprises SEQ ID NO:19, a contiguous portion thereof of at least 14 nt, or a complementary sequence of thereof. In some embodiments, the target site comprises SEQ ID NO:20, a contiguous portion thereof of at least 14 nt, or a complementary sequence of thereof. In some embodiments, the target site comprises SEQ ID NO:21, a contiguous portion thereof of at least 14 nt, or a complementary sequence of thereof.

In some embodiments, the target site comprises SEQ ID NO:229, a contiguous portion thereof of at least 14 nt, or a complementary sequence of thereof. In some embodiments, the target site comprises SEQ ID NO:230, a contiguous portion thereof of at least 14 nt, or a complementary sequence of thereof. In some embodiments, the target site comprises SEQ ID NO:231, a contiguous portion thereof of at least 14 nt, or a complementary sequence of thereof. In some embodiments, the target site comprises SEQ ID NO:232, a contiguous portion thereof of at least 14 nt, or a complementary sequence of thereof. In some embodiments, the target site comprises SEQ ID NO:233, a contiguous portion thereof of at least 14 nt, or a complementary sequence of thereof. In some embodiments, the target site comprises SEQ ID NO:234, a contiguous portion thereof of at least 14 nt, or a complementary sequence of thereof. In some embodiments, the target site comprises SEQ ID NO:235, a contiguous portion thereof of at least 14 nt, or a complementary sequence of thereof. In some embodiments, the target site comprises SEQ ID NO:236, a contiguous portion thereof of at least 14 nt, or a complementary sequence of thereof. In some embodiments, the target site comprises SEQ ID NO:237, a contiguous portion thereof of at least 14 nt, or a complementary sequence of thereof. In some embodiments, the target site comprises SEQ ID NO:238, a contiguous portion thereof of at least 14 nt, or a complementary sequence of thereof. In some embodiments, the target site comprises SEQ ID NO:239, a contiguous portion thereof of at least 14 nt, or a complementary sequence of thereof. In some embodiments, the target site comprises SEQ ID NO:240, a contiguous portion thereof of at least 14 nt, or a complementary sequence of thereof. In some embodiments, the target site comprises SEQ ID NO:241, a contiguous portion thereof of at least 14 nt, or a complementary sequence of thereof. In some embodiments, the target site comprises SEQ ID NO:242, a contiguous portion thereof of at least 14 nt, or a complementary sequence of thereof. In some embodiments, the target site comprises SEQ ID NO:243, a contiguous portion thereof of at least 14 nt, or a complementary sequence of thereof. In some embodiments, the target site comprises SEQ ID NO:244, a contiguous portion thereof of at least 14 nt, or a complementary sequence of thereof. In some embodiments, the target site comprises SEQ ID NO:245, a contiguous portion thereof of at least 14 nt, or a complementary sequence of thereof. In some embodiments, the target site comprises SEQ ID NO:246, a contiguous portion thereof of at least 14 nt, or a complementary sequence of thereof. In some embodiments, the target site comprises SEQ ID NO:247, a contiguous portion thereof of at least 14 nt, or a complementary sequence of thereof. In some embodiments, the target site comprises SEQ ID NO:248, a contiguous portion thereof of at least 14 nt, or a complementary sequence of thereof. In some embodiments, the target site comprises SEQ ID NO:249, a contiguous portion thereof of at least 14 nt, or a complementary sequence of thereof. In some embodiments, the target site comprises SEQ ID NO:250, a contiguous portion thereof of at least 14 nt, or a complementary sequence of thereof. In some embodiments, the target site comprises SEQ ID NO:251, a contiguous portion thereof of at least 14 nt, or a complementary sequence of thereof. In some embodiments, the target site comprises SEQ ID NO:252, a contiguous portion thereof of at least 14 nt, or a complementary sequence of thereof. In some embodiments, the target site comprises SEQ ID NO:253, a contiguous portion thereof of at least 14 nt, or a complementary sequence of thereof. In some embodiments, the target site comprises SEQ ID NO:254, a contiguous portion thereof of at least 14 nt, or a complementary sequence of thereof. In some embodiments, the target site comprises SEQ ID NO:255, a contiguous portion thereof of at least 14 nt, or a complementary sequence of thereof. In some embodiments, the target site comprises SEQ ID NO:256, a contiguous portion thereof of at least 14 nt, or a complementary sequence of thereof. In some embodiments, the target site comprises SEQ ID NO:257, a contiguous portion thereof of at least 14 nt, or a complementary sequence of thereof. In some embodiments, the target site comprises SEQ ID NO:258, a contiguous portion thereof of at least 14 nt, or a complementary sequence of thereof. In some embodiments, the target site comprises SEQ ID NO:259, a contiguous portion thereof of at least 14 nt, or a complementary sequence of thereof. In some embodiments, the target site comprises SEQ ID NO:260, a contiguous portion thereof of at least 14 nt, or a complementary sequence of thereof. In some embodiments, the target site comprises SEQ ID NO:261, a contiguous portion thereof of at least 14 nt, or a complementary sequence of thereof. In some embodiments, the target site comprises SEQ ID NO:262, a contiguous portion thereof of at least 14 nt, or a complementary sequence of thereof. In some embodiments, the target site comprises SEQ ID NO:263, a contiguous portion thereof of at least 14 nt, or a complementary sequence of thereof. In some embodiments, the target site comprises SEQ ID NO:264, a contiguous portion thereof of at least 14 nt, or a complementary sequence of thereof. In some embodiments, the target site comprises SEQ ID NO:265, a contiguous portion thereof of at least 14 nt, or a complementary sequence of thereof.

In some embodiments, the target site comprises SEQ ID NO:1. In some embodiments, the target site comprises SEQ ID NO:2. In some embodiments, the target site comprises SEQ ID NO:3. In some embodiments, the target site comprises SEQ ID NO:4. In some embodiments, the target site comprises SEQ ID NO:5. In some embodiments, the target site comprises SEQ ID NO:6. In some embodiments, the target site comprises SEQ ID NO:7. In some embodiments, the target site comprises SEQ ID NO:8. In some embodiments, the target site comprises SEQ ID NO:9. In some embodiments, the target site comprises SEQ ID NO: 10. In some embodiments, the target site comprises SEQ ID NO:11. In some embodiments, the target site comprises SEQ ID NO:12. In some embodiments, the target site comprises SEQ ID NO:13. In some embodiments, the target site comprises SEQ ID NO:14. In some embodiments, the target site comprises SEQ ID NO:15. In some embodiments, the target site comprises SEQ ID NO:16. In some embodiments, the target site comprises SEQ ID NO:17. In some embodiments, the target site comprises SEQ ID NO: 18. In some embodiments, the target site comprises SEQ ID NO: 19. In some embodiments, the target site comprises SEQ ID NO:20. In some embodiments, the target site comprises SEQ ID NO:21.

In some embodiments, the target site comprises SEQ ID NO:229. In some embodiments, the target site comprises SEQ ID NO:230. In some embodiments, the target site comprises SEQ ID NO:231. In some embodiments, the target site comprises SEQ ID NO:232. In some embodiments, the target site comprises SEQ ID NO:233. In some embodiments, the target site comprises SEQ ID NO:234. In some embodiments, the target site comprises SEQ ID NO:235. In some embodiments, the target site comprises SEQ ID NO:236. In some embodiments, the target site comprises SEQ ID NO:237. In some embodiments, the target site comprises SEQ ID NO:238. In some embodiments, the target site comprises SEQ ID NO:239. In some embodiments, the target site comprises SEQ ID NO:240. In some embodiments, the target site comprises SEQ ID NO:241. In some embodiments, the target site comprises SEQ ID NO:242. In some embodiments, the target site comprises SEQ ID NO:243. In some embodiments, the target site comprises SEQ ID NO:244. In some embodiments, the target site comprises SEQ ID NO:245. In some embodiments, the target site comprises SEQ ID NO:246. In some embodiments, the target site comprises SEQ ID NO:247. In some embodiments, the target site comprises SEQ ID NO:248. In some embodiments, the target site comprises SEQ ID NO:249. In some embodiments, the target site comprises SEQ ID NO:250. In some embodiments, the target site comprises SEQ ID NO:251. In some embodiments, the target site comprises SEQ ID NO:252. In some embodiments, the target site comprises SEQ ID NO:253. In some embodiments, the target site comprises SEQ ID NO:254. In some embodiments, the target site comprises SEQ ID NO:255. In some embodiments, the target site comprises SEQ ID NO:256. In some embodiments, the target site comprises SEQ ID NO:257. In some embodiments, the target site comprises SEQ ID NO:258. In some embodiments, the target site comprises SEQ ID NO:259. In some embodiments, the target site comprises SEQ ID NO:260. In some embodiments, the target site comprises SEQ ID NO:261. In some embodiments, the target site comprises SEQ ID NO:262. In some embodiments, the target site comprises SEQ ID NO:263. In some embodiments, the target site comprises SEQ ID NO:264. In some embodiments, the target site comprises SEQ ID NO:265.

In some embodiments, the target site comprises a complementary sequence of SEQ ID NO:1. In some embodiments, the target site comprises a complementary sequence of SEQ ID NO:2. In some embodiments, the target site comprises a complementary sequence of SEQ ID NO:3. In some embodiments, the target site comprises a complementary sequence of SEQ ID NO:4. In some embodiments, the target site comprises a complementary sequence of SEQ ID NO:5. In some embodiments, the target site comprises a complementary sequence of SEQ ID NO:6. In some embodiments, the target site comprises a complementary sequence of SEQ ID NO:7. In some embodiments, the target site comprises a complementary sequence of SEQ ID NO:8. In some embodiments, the target site comprises a complementary sequence of SEQ ID NO:9. In some embodiments, the target site comprises a complementary sequence of SEQ ID NO:10. In some embodiments, the target site comprises a complementary sequence of SEQ ID NO:11. In some embodiments, the target site comprises a complementary sequence of SEQ ID NO:12. In some embodiments, the target site comprises a complementary sequence of SEQ ID NO:13. In some embodiments, the target site comprises a complementary sequence of SEQ ID NO:14. In some embodiments, the target site comprises a complementary sequence of SEQ ID NO:15. In some embodiments, the target site comprises a complementary sequence of SEQ ID NO:16. In some embodiments, the target site comprises a complementary sequence of SEQ ID NO:17. In some embodiments, the target site comprises a complementary sequence of SEQ ID NO:18. In some embodiments, the target site comprises a complementary sequence of SEQ ID NO:19. In some embodiments, the target site comprises a complementary sequence of SEQ ID NO:20. In some embodiments, the target site comprises a complementary sequence of SEQ ID NO:21.

In some embodiments, the target site comprises the sequence set forth in SEQ ID NO:1, SEQ ID NO:7, or SEQ ID NO:21, a contiguous portion thereof of at least 14 nt, or a complementary sequence of any of the foregoing. In some embodiments, the target site comprises the sequence set forth in SEQ ID NO:1, a contiguous portion thereof of at least 14 nt, or a complementary sequence of any of the foregoing. In some embodiments, the target site comprises the sequence set forth in SEQ ID NO:7, a contiguous portion thereof of at least 14 nt, or a complementary sequence of any of the foregoing. In some embodiments, the target site comprises the sequence set forth in SEQ ID NO:21, a contiguous portion thereof of at least 14 nt, or a complementary sequence of any of the foregoing. In some embodiments, the target site comprises the sequence set forth in SEQ ID NO:1. In some embodiments, the target site comprises the sequence set forth in SEQ ID NO:7. In some embodiments, the target site comprises the sequence set forth in SEQ ID NO:21. In some embodiments, the target site comprises a complementary sequence of the sequence set forth in SEQ ID NO:1. In some embodiments, the target site comprises a complementary sequence of the sequence set forth in SEQ ID NO:7. In some embodiments, the target site comprises a complementary sequence of the sequence set forth in SEQ ID NO:21.

B. Guide RNAs (gRNAs)

Provided herein are gRNAs, such as gRNAs that target or can bind to a regulatory DNA element of a FXN locus. In some embodiments, the gRNA is capable of complexing with the Cas protein or variant thereof. In some embodiments, the gRNA comprises a gRNA spacer sequence (also known as a spacer sequence or a guide sequence) that is capable of hybridizing to the target site or is complementary to the target site, such as any target site described herein, for example, any target site in a genome. In some embodiments, the gRNA comprises a scaffold sequence that complexes with or binds to the Cas protein. In some embodiments, a gRNA specific to a target locus of interest (e.g. a regulatory DNA element of a FXN locus) is used to recruit an RNA-guided protein (e.g. a Cas protein) or variant thereof or a fusion protein comprising such RNA-guided protein (e.g., a Cas polypeptide), to the target site.

In some embodiments, the Cas protein (e.g. dCas9) is provided in combination or as a complex with one or more guide RNA (gRNA). In some aspects, the gRNA is a nucleic acid that promotes the specific targeting or homing of the gRNA/Cas RNP complex to the target site, such as any described above. In some embodiments, a target site of a gRNA may be referred to as a protospacer.

Provided herein are gRNAs, such as gRNAs that target or bind to a target site in a FXN gene or DNA regulatory element thereof, such as any described above in Section I.A. In some embodiments, the gRNA is capable of complexing with the Cas protein or variant thereof. In some embodiments, the gRNA comprises a gRNA spacer sequence (i.e. a spacer sequence or a guide sequence) that is capable of hybridizing to the target site, or that is complementary to the target site, such as any target site described in Section I.A or further below. In some embodiments, the gRNA comprises a scaffold sequence that complexes with or binds to the Cas protein.

In some aspects, a “gRNA molecule” is a nucleic acid that promotes the specific targeting or homing of a gRNA molecule/Cas9 molecule complex to a target nucleic acid, such as a locus on the genomic DNA of a cell. gRNA molecules can be unimolecular (having a single RNA molecule), sometimes referred to herein as “chimeric” gRNAs, or modular (comprising more than one, and typically two, separate RNA molecules). In general, a spacer sequence of the guide RNA, is any polynucleotide sequences comprising at least a sequence portion that has sufficient complementarity with a target polynucleotide sequence, such as the at the FXN locus in humans, to hybridize with the target sequence at the target site and direct sequence-specific binding of the CRISPR complex to the target sequence. In some embodiments, in the context of formation of a CRISPR complex, “target sequence” is to a sequence to which a spacer sequence is designed to have complementarity, where hybridization between the target sequence and a spacer sequence of the guide RNA promotes the formation of a CRISPR complex. Full complementarity is not necessarily required, provided there is sufficient complementarity to cause hybridization and promote formation of a CRISPR complex. Generally, a spacer sequence is selected to reduce the degree of secondary structure within the spacer sequence. Secondary structure may be determined by any suitable polynucleotide folding algorithm.

In some embodiments, a guide RNA (gRNA) specific to a target locus of interest (e.g. at the FXN locus in humans) is used with RNA-guided nucleases or variants thereof, e.g., nuclease-inactive Cas variants, to target the provided DNA-targeting system to the target site or target position. Methods for designing gRNAs and exemplary spacer sequences are known. Exemplary gRNA structures that can be associated with particular RNA-guided nucleases or variants thereof, e.g., nuclease-inactive Cas variants, with particular domains and scaffold regions, are also known. In some aspects, gRNA molecules comprise a scaffold sequence, e.g., sequences that can be complexed with the Cas protein. In some aspects, the scaffold sequence is specific for the Cas protein.

In some embodiments, the gRNA is a chimeric gRNA. In general, gRNAs can be unimolecular (i.e. composed of a single RNA molecule), or modular (comprising more than one, and typically two, separate RNA molecules). Modular gRNAs can be engineered to be unimolecular, wherein sequences from the separate modular RNA molecules are comprised in a single gRNA molecule, sometimes referred to as a chimeric gRNA, synthetic gRNA, or single gRNA. A guide RNA can comprise at least a spacer sequence that hybridizes to a target nucleic acid sequence of interest, and a CRISPR repeat sequence. In Type II systems, the gRNA also comprises a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex can bind a site-directed polypeptide, such that the guide RNA and site-direct polypeptide form a complex. The gRNA can provide target specificity to the complex by virtue of its association with the site-directed polypeptide. The gRNA thus can direct the activity of the site-directed polypeptide.

In some embodiments, the chimeric gRNA is a fusion of two non-coding RNA sequences: a crRNA sequence and a tracrRNA sequence, for example as described in WO 2013/176772, or Jinek, M. et al. Science 337(6096):816-21 (2012). In some embodiments, the chimeric gRNA mimics the naturally occurring crRNA:tracrRNA duplex involved in the Type II CRISPR/Cas system, wherein the naturally occurring crRNA:tracrRNA duplex acts as a guide for the Cas protein, e.g., Cas9 protein. Exemplary types of CRISPR/Cas systems and associated gRNA structures include those described in, for example, Moon et al. Exp. Mol. Med. 51, 1-11 (2019), Zhang, F. Q. Rev. Biophys. 52, E6 (2019), Makarova et al. Methods Mol. Biol. 1311:47-75 (2015), WO 2013/176772, or Jinck, M. et al. Science 337(6096):816-21 (2012).

In some aspects, the spacer sequence of a gRNA is a polynucleotide sequence comprising at least a portion that has sufficient complementarity with the target site to hybridize with the target site and direct sequence-specific binding of a CRISPR complex to the sequence of the target site. Full complementarity is not necessarily required, provided there is sufficient complementarity to cause hybridization and promote formation of a CRISPR complex. In some embodiments, the gRNA comprises a spacer sequence that is complementary, e.g., at least 80%, 85%, 90%, 95%, 98%, 99%, or 100% (e.g., fully complementary), to the target site. The strand of the target nucleic acid comprising the target site sequence may be referred to as the “complementary strand” of the target nucleic acid. In some aspects, the spacer sequence is a user-defined sequence. Guidance on the selection of spacer sequences can be found, e.g., in Fu et al., Nat Biotechnol 2014 32:279-284 and Sternberg et al., Nature 2014 507:62-67.

In some embodiments, the gRNA spacer sequence is between about 14 nt and about 26 nt, between about 14 nt and about 24 nt, or between 16 nt and 22 nt in length. In some embodiments, the gRNA spacer sequence is 14 nt, 15 nt, 16 nt, 17 nt, 18 nt, 19 nt, 20 nt, 21 nt or 22 nt, 23 nt, 24 nt, 25 nt, or 26 nt in length. In some embodiments, the gRNA spacer sequence is 18 nt, 19 nt, 20 nt, 21 nt or 22 nt in length. In some embodiments, the gRNA spacer sequence is 18 nt in length. In some embodiments, the gRNA spacer sequence is 19 nt in length. In some embodiments, the gRNA spacer sequence is 20 nt in length. In some embodiments, the gRNA spacer sequence is 21 nt in length. In some embodiments, the gRNA spacer sequence is 22 nt in length.

Methods for designing gRNAs and exemplary targeting domains can include those described in, e.g., International PCT Pub. Nos. WO 2014/197748, WO 2016/130600, WO 2017/180915, WO 2021/226555, WO 2013/176772, WO 2014/152432, WO 2014/093661, WO 2014/093655, WO 2015/089427, WO 2016/049258, WO 2016/123578, WO 2021/076744, WO 2014/191128, WO 2015/161276, WO 2017/193107, and WO 2017/093969.

A target site of a gRNA may be referred to as a protospacer. In some aspects, the spacer is designed to target a protospacer with a specific protospacer-adjacent motif (PAM), i.e. a sequence immediately adjacent to the protospacer that contributes to and/or is required for Cas binding specificity. Different CRISPR/Cas systems have different PAM requirements for targeting. For example, in some embodiments, S. pyogenes Cas9 uses the PAM 5′-NGG-3′ (SEQ ID NO: 142), where N is any nucleotide. S. aureus Cas9 uses the PAM 5′-NNGRRT-3′ (SEQ ID NO:143), where N is any nucleotide, and R is G or A. N. meningitidis Cas9 uses the PAM 5′-NNNNGATT-3′ (SEQ ID NO:144), where N is any nucleotide. C. jejuni Cas9 uses the PAM 5′-NNNNRYAC-3′ (SEQ ID NO:145) or 5′-NNNNACAC-3′(SEQ ID NO:226), where N is any nucleotide, R is G or A, and Y is C or T. S. thermophilus uses the PAM 5′-NNAGAAW-3′ (SEQ ID NO: 146), where N is any nucleotide and W is A or T. F. Novicida Cas9 uses the PAM 5′-NGG-3′ (SEQ ID NO:142), where N is any nucleotide. T. denticola Cas9 uses the PAM 5′-NAAAAC-3′ (SEQ ID NO:146), where N is any nucleotide. Cas12a (also known as Cpf1) from various species, uses the PAM 5′-TTTV-3′ (SEQ ID NO:148), where V is A, C, or G. Phage-derived CasPhi (such as CasPhi-2, also known as Cas12j), uses the PAM 5′-TBN-3′ (SEQ ID NO:224), where N is any nucleotide, and B is G, T, or C. Archacal Un1Cas12f1 (also known as Cas14a1), uses the PAM 5′-TTTN-3′ (SEQ ID NO:225), where N is any nucleotide. A Cas12f protein (also known as Cas14) uses the PAM 5′-TTTR-3′ (SEQ ID NO:228), where R is G or A. A Cas12k protein uses the PAM 5′-GGTT-3′ (SEQ ID NO:227). Cas proteins may use or be engineered to use different PAMs from those listed above. For example, variant SpCas9 proteins may use a PAM selected from: 5′-NGG-3′ (SEQ ID NO:142), 5′-NGAN-3′ (SEQ ID NO:149), 5′-NGNG-3′(SEQ ID NO:150), 5′-NGAG-3′(SEQ ID NO:151), or 5′-NGCG-3′(SEQ ID NO:152), where N is any nucleotide. Methods for designing or identifying gRNA spacer sequences and/or protospacer sequences in a particular region, are known. gRNA spacer sequences and/or protospacer sequences can be determined based on the type of Cas protein used and the associated PAM sequence.

In some embodiments, the PAM of a gRNA for complexing with S. pyogenes Cas9 or variant thereof is set forth in SEQ ID NO:141. In some embodiments, the PAM of a gRNA for complexing with S. aureus Cas9 or variant thereof is set forth in SEQ ID NO: 142. In some embodiments, the PAM of a gRNA for complexing with a Type V CRISPR/Cas system, such as with Cas12a (also known as Cpf1) or variant thereof is set forth in SEQ ID NO:147.

A spacer sequence may be selected to reduce the degree of secondary structure within the spacer sequence. Secondary structure may be determined by any suitable polynucleotide folding algorithm.

In some embodiments, the gRNA (including the spacer sequence) will comprise the base uracil (U), whereas DNA encoding the gRNA molecule will comprise the base thymine (T). While not wishing to be bound by theory, in some embodiments, it is believed that the complementarity of the spacer sequence (i.e. guide sequence) with the target sequence contributes to specificity of the interaction of the gRNA molecule/Cas molecule complex with a target nucleic acid. It is understood that in a spacer sequence (i.e. guide sequence) and target sequence pair, the uracil bases in the spacer sequence (i.e. guide sequence) will pair with the adenine bases in the target sequence. A gRNA spacer sequence herein may be defined by the DNA sequence encoding the gRNA spacer, and/or the RNA sequence of the spacer.

In some embodiments, the gRNA comprises modified nucleotides, e.g. for increased stability. In some embodiments, one, more than one, or all of the nucleotides of a gRNA can have a modification, e.g., to render the gRNA less susceptible to degradation and/or improve bio-compatibility. By way of non-limiting example, the backbone of the gRNA can be modified with a phosphorothioate, or other modification(s). In some cases, a nucleotide of the gRNA can comprise a 2′ modification, e.g., a 2-acetylation, e.g., a 2′ methylation, or other modification(s)

In some embodiments the gRNA is a concatenation of two non-coding RNA sequences: a crRNA sequence and a tracrRNA sequence. The gRNA may target a desired DNA sequence by exchanging the sequence encoding a 20 bp protospacer which confers targeting specificity through complementary base pairing with the desired DNA target. gRNA mimics the naturally occurring crRNA:tracrRNA duplex involved in the Type II CRISPR/Cas system (e.g., Cas9). This duplex, which may include, for example, a 42-nucleotide crRNA and a 75-nucleotide tracrRNA, acts as a guide for the Cas9 protein to cleave the target nucleic acid. The “target region”, “target sequence” or “protospacer” as used interchangeably herein refers to the region of the target gene to which the CRISPR/Cas9-based system targets. The CRISPR/Cas9-based system may include two or more gRNAs, wherein the two or more gRNAs target different DNA sequences. The target DNA sequences may be overlapping or non-overlapping. The target DNA sequences may be located within or near the same gene or different genes. The target sequence or protospacer is followed by a PAM sequence at the 3′ end of the protospacer. Different Type II systems have differing PAM requirements. For example, the Streptococcus pyogenes Type II system uses an “NGG” sequence, where “N” can be any nucleotide.

In some aspects, the gRNA comprises scaffold sequences. In some aspects, the scaffold sequence (in some cases including a crRNA sequence and/or a tracrRNA sequence) will be different depending on the Cas protein. In some aspects, different CRISPR/Cas systems have different gRNA scaffold sequences for associating with Cas protein. In some embodiments, an exemplary scaffold sequence for S. aureus Cas9 comprises a sequence set forth in SEQ ID NO:44, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:44. In some embodiments, an exemplary scaffold sequence for S. aureus Cas9 comprises a sequence set forth in SEQ ID NO:44. In some embodiments, an exemplary scaffold sequence for S. pyogenes Cas9 comprises a sequence set forth in SEQ ID NO:46, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:46. In some embodiments, an exemplary scaffold sequence for S. pyogenes Cas9 comprises a sequence set forth in SEQ ID NO:46. In some embodiments, an exemplary scaffold sequence for Acidaminococcus sp. Cas12a comprises a sequence set forth in SEQ ID NO:211, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:211. In some embodiments, an exemplary scaffold sequence for CasPhi-2 comprises a sequence set forth in SEQ ID NO:212, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:212. In some embodiments, an exemplary scaffold sequence for Un1Cas12f1 comprises a sequence set forth in SEQ ID NO:213, 214 or 215, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:213, 214 or 215. In some embodiments, an exemplary scaffold sequence for Un1Cas12f1 comprises a sequence set forth in SEQ ID NO:213, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:213. In some embodiments, an exemplary scaffold sequence for Un1Cas12f1 comprises a sequence set forth in SEQ ID NO:214, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:214. In some embodiments, an exemplary scaffold sequence for Un1Cas12f1 comprises a sequence set forth in SEQ ID NO:215, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:215. In some embodiments, an exemplary scaffold sequence for C. jejuni Cas9 comprises a sequence set forth in SEQ ID NO:216, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:216. In some embodiments, an exemplary scaffold sequence for Cas12k comprises a sequence set forth in SEQ ID NO:217, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:217. In some embodiments, an exemplary scaffold sequence for CasMini comprises a sequence set forth in SEQ ID NO:218, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:218.

In some aspects, the gRNA can target the DNA-targeting system to direct the activities of an associated polypeptide (e.g., fusion protein, DNA-targeting system, effector domain, etc.) to a specific target site within a target nucleic acid (e.g., regulatory DNA element of a FXN locus).

In some aspects, provided herein is a guide RNA (gRNA) that binds a target site in an enhancer region of a frataxin (FXN) locus, wherein the target site is located within the genomic coordinates human genome assembly GRCh38 (hg38) chr9:69,027,282-69,028,497. In some aspects, provided herein is a guide RNA (gRNA) that binds a target site in an enhancer region of a frataxin (FXN) locus, wherein the target site is located within the genomic coordinates hg38 chr9:69,027,615-69,028,101.

In some embodiments the target site comprises the sequence set forth in SEQ ID NO:21, a contiguous portion thereof of at least 14 nt, or a complementary sequence of any of the foregoing. In some embodiments DNA encoding the gRNA comprises the sequence set forth in SEQ ID NO:21, a contiguous portion thereof of at least 14 nucleotides, or a complementary sequence of any of the foregoing. In some embodiments the gRNA comprises at least one gRNA spacer comprising the sequence set forth in SEQ ID NO:42, or a contiguous portion thereof of at least 14 nt. In some embodiments the gRNA further comprises the sequence set forth in SEQ ID NO:44. In some embodiments the gRNA comprises the sequence set forth in SEQ ID NO:67.

Also provided herein is a guide RNA (gRNA) that binds a target site in a regulatory DNA element of a frataxin (FXN) locus, such as a FXN promoter, wherein the target site comprises a sequence selected from any one of SEQ ID NOS: 1-10, a contiguous portion thereof of at least 14 nt, or a complementary sequence of any of the foregoing.

In some of any of the provided embodiments, the gRNA comprises at least one gRNA spacer sequence comprising a sequence selected from any one of SEQ ID NOS:22-31, or a contiguous portion thereof of at least 14 nt. In some of any of the provided embodiments, the gRNA further comprises the sequence set forth in SEQ ID NO:44. In some of any of the provided embodiments, the gRNA comprises a sequence selected from any one of SEQ ID NOS:47-56.

Also provided herein is a guide RNA (gRNA) that binds a target site in a regulatory DNA element of a frataxin (FXN) locus, such as a FXN promoter, wherein the target site comprises a sequence selected from any one of SEQ ID NOS:11-20, a contiguous portion thereof of at least 14 nt, or a complementary sequence of any of the foregoing.

In some of any of the provided embodiments, the gRNA comprises at least one gRNA spacer sequence comprising a sequence selected from any one of SEQ ID NOS:32-41, or a contiguous portion thereof of at least 14 nt. In some of any of the provided embodiments, the gRNA further comprises the sequence set forth in SEQ ID NO:46. In some of any of the provided embodiments, the gRNA comprises a sequence selected from any one of SEQ ID NOS:57-66.

In some embodiments, the DNA-targeting domain comprises a Cas-gRNA combination comprising (a) a Cas protein or a variant thereof and (b) at least one gRNA; and the gRNA comprises at least one gRNA spacer sequence comprising a sequence selected from any one of SEQ ID NOS:22-31, or a contiguous portion thereof of at least 14 nt. In some embodiments, the gRNA further comprises the sequence set forth in SEQ ID NO:44. In some embodiments, the gRNA comprises a sequence selected from any one of SEQ ID NOS:47-56.

In some embodiments, the DNA-targeting domain comprises a Cas-gRNA combination comprising (a) a Cas protein or a variant thereof and (b) at least one gRNA; and the gRNA comprises at least one gRNA spacer sequence comprising a sequence selected from any one of SEQ ID NOS:32-41, or a contiguous portion thereof of at least 14 nt. In some embodiments, the gRNA further comprises the sequence set forth in SEQ ID NO:46. In some embodiments, the gRNA comprises a sequence selected from any one of SEQ ID NOS:57-66.

In some embodiments, the gRNA comprises a sequence having at or at least 80%, 85%, 90%, 91%, 92%, 93%, 94%, 95%, 96%, 97%, 98%, 99%, 99.5%, 99.9%, or 100% sequence identity to all or a portion of a gRNA sequence or a gRNA spacer sequence described herein.

In some embodiments, the gRNA targets a target site in a FXN locus or a DNA regulatory element thereof that comprises the sequence selected from any one of SEQ ID NO:1-10 and 21, a contiguous portion thereof of at least 14 nucleotides (e.g., 14, 15, 16, 17, 18, 19, 20, 21, or 22 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising the sequence selected from any one of SEQ ID NO:22-31 and 42, a contiguous portion thereof of at least 14 nt (e.g., 14, 15, 16, 17, 18, 19, 20, 21, or 22 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO:44, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:44. In some embodiments, the gRNA, including a spacer sequence and a scaffold sequence, comprises the sequence selected from any one of SEQ ID NO:47-56 and 67, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA is set forth in the sequence selected from any one of SEQ ID NO:47-56 and 67. In some embodiments, a provided DNA-targeting system comprises any of the aforementioned gRNAs complexed with a Cas protein, such as a Cas9 protein. In some embodiments, the Cas9 is a dCas9. In some embodiments, the dCas9 is a dSaCas9, such as a dSaCas9 set forth in SEQ ID NO:72, or a variant and/or fusion thereof.

In some embodiments, the gRNA targets a target site in a FXN locus or a DNA regulatory element thereof that comprises the sequence selected from any one of SEQ ID NO:229-255, a contiguous portion thereof of at least 14 nucleotides (e.g., 14, 15, 16, 17, 18, 19, 20, 21, or 22 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO:211, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:211. In some embodiments, the gRNA comprises, in 5′ to 3′ order, a spacer targeting SEQ ID NO:229, and a scaffold sequence of SEQ ID NO:211. In some embodiments, the gRNA comprises, in 5′ to 3′ order, a spacer targeting SEQ ID NO:230, and a scaffold sequence of SEQ ID NO:211. In some embodiments, the gRNA comprises, in 5′ to 3′ order, a spacer targeting SEQ ID NO:231, and a scaffold sequence of SEQ ID NO:211. In some embodiments, the gRNA comprises, in 5′ to 3′ order, a spacer targeting SEQ ID NO:232, and a scaffold sequence of SEQ ID NO:211. In some embodiments, the gRNA comprises, in 5′ to 3′ order, a spacer targeting SEQ ID NO:233, and a scaffold sequence of SEQ ID NO:211. In some embodiments, the gRNA comprises, in 5′ to 3′ order, a spacer targeting SEQ ID NO:234, and a scaffold sequence of SEQ ID NO:211. In some embodiments, the gRNA comprises, in 5′ to 3′ order, a spacer targeting SEQ ID NO:235, and a scaffold sequence of SEQ ID NO:211. In some embodiments, the gRNA comprises, in 5′ to 3′ order, a spacer targeting SEQ ID NO:236, and a scaffold sequence of SEQ ID NO:211. In some embodiments, the gRNA comprises, in 5′ to 3′ order, a spacer targeting SEQ ID NO:237, and a scaffold sequence of SEQ ID NO:211. In some embodiments, the gRNA comprises, in 5′ to 3′ order, a spacer targeting SEQ ID NO:238, and a scaffold sequence of SEQ ID NO:211. In some embodiments, the gRNA comprises, in 5′ to 3′ order, a spacer targeting SEQ ID NO:239, and a scaffold sequence of SEQ ID NO:211. In some embodiments, the gRNA comprises, in 5′ to 3′ order, a spacer targeting SEQ ID NO:240, and a scaffold sequence of SEQ ID NO:211. In some embodiments, the gRNA comprises, in 5′ to 3′ order, a spacer targeting SEQ ID NO:241, and a scaffold sequence of SEQ ID NO:211. In some embodiments, the gRNA comprises, in 5′ to 3′ order, a spacer targeting SEQ ID NO:242, and a scaffold sequence of SEQ ID NO:211. In some embodiments, the gRNA comprises, in 5′ to 3′ order, a spacer targeting SEQ ID NO:243, and a scaffold sequence of SEQ ID NO:211. In some embodiments, the gRNA comprises, in 5′ to 3′ order, a spacer targeting SEQ ID NO:244, and a scaffold sequence of SEQ ID NO:211. In some embodiments, the gRNA comprises, in 5′ to 3′ order, a spacer targeting SEQ ID NO:245, and a scaffold sequence of SEQ ID NO:211. In some embodiments, the gRNA comprises, in 5′ to 3′ order, a spacer targeting SEQ ID NO:246, and a scaffold sequence of SEQ ID NO:211. In some embodiments, the gRNA comprises, in 5′ to 3′ order, a spacer targeting SEQ ID NO:247, and a scaffold sequence of SEQ ID NO:211. In some embodiments, the gRNA comprises, in 5′ to 3′ order, a spacer targeting SEQ ID NO:248, and a scaffold sequence of SEQ ID NO:211. In some embodiments, the gRNA comprises, in 5′ to 3′ order, a spacer targeting SEQ ID NO:249, and a scaffold sequence of SEQ ID NO:211. In some embodiments, the gRNA comprises, in 5′ to 3′ order, a spacer targeting SEQ ID NO:250, and a scaffold sequence of SEQ ID NO:211. In some embodiments, the gRNA comprises, in 5′ to 3′ order, a spacer targeting SEQ ID NO:251, and a scaffold sequence of SEQ ID NO:211. In some embodiments, the gRNA comprises, in 5′ to 3′ order, a spacer targeting SEQ ID NO:252, and a scaffold sequence of SEQ ID NO:211. In some embodiments, the gRNA comprises, in 5′ to 3′ order, a spacer targeting SEQ ID NO:253, and a scaffold sequence of SEQ ID NO:211. In some embodiments, the gRNA comprises, in 5′ to 3′ order, a spacer targeting SEQ ID NO:254, and a scaffold sequence of SEQ ID NO:211. In some embodiments, the gRNA comprises, in 5′ to 3′ order, a spacer targeting SEQ ID NO:255, and a scaffold sequence of SEQ ID NO:211. In some embodiments, a provided DNA-targeting system comprises any of the aforementioned gRNAs complexed with a Cas protein, such as a Cas12a (also known as Cpf1) protein. In some embodiments, the Cas12a is a dCas12a. In some embodiments, the dCas12a is a dSaCas12a, such as a dSaCas12a set forth in SEQ ID NO:192, or a variant and/or fusion thereof.

In some aspects, the gRNA targets a target site in a FXN locus that comprises SEQ ID NO:1, a contiguous portion thereof of at least 14 nucleotides, a complementary sequence of any of the foregoing, or a sequence having at least 90% sequence identity to any of the foregoing. In some aspects, the gRNA comprises a spacer sequence comprising SEQ ID NO:22, a contiguous portion thereof of at least 14 nt, or a sequence having at least 90% sequence identity to any of the foregoing. In some aspects, the gRNA further comprises a scaffold sequence comprising SEQ ID NO:44, or a sequence having at least 90% sequence identity to SEQ ID NO:44. In some aspects, the gRNA, including a spacer sequence and a scaffold sequence, comprises SEQ ID NO:47, or a sequence having at least 90% sequence identity to all or a portion thereof. In some aspects, the gRNA is set forth in SEQ ID NO:47. In some aspects, a provided DNA-targeting system comprises any of the above gRNAs complexed with a Cas protein, such as a Cas9 protein. In some aspects, the Cas9 is a dCas9. In some aspects, the dCas9 is a dSaCas9, such as a dSaCas9 set forth in SEQ ID NO:72, or a variant and/or fusion thereof.

In some aspects, the gRNA targets a target site in a FXN locus that comprises SEQ ID NO:2, a contiguous portion thereof of at least 14 nucleotides, a complementary sequence of any of the foregoing, or a sequence having at least 90% sequence identity to any of the foregoing. In some aspects, the gRNA comprises a spacer sequence comprising SEQ ID NO:23, a contiguous portion thereof of at least 14 nt, or a sequence having at least 90% sequence identity to any of the foregoing. In some aspects, the gRNA further comprises a scaffold sequence comprising SEQ ID NO:44, or a sequence having at least 90% sequence identity to SEQ ID NO:44. In some aspects, the gRNA, including a spacer sequence and a scaffold sequence, comprises SEQ ID NO:48, or a sequence having at least 90% sequence identity to all or a portion thereof. In some aspects, the gRNA is set forth in SEQ ID NO:48. In some aspects, a provided DNA-targeting system comprises any of the above gRNAs complexed with a Cas protein, such as a Cas9 protein. In some aspects, the Cas9 is a dCas9. In some aspects, the dCas9 is a dSaCas9, such as a dSaCas9 set forth in SEQ ID NO:72, or a variant and/or fusion thereof.

In some aspects, the gRNA targets a target site in a FXN locus that comprises SEQ ID NO:3, a contiguous portion thereof of at least 14 nucleotides, a complementary sequence of any of the foregoing, or a sequence having at least 90% sequence identity to any of the foregoing. In some aspects, the gRNA comprises a spacer sequence comprising SEQ ID NO:24, a contiguous portion thereof of at least 14 nt, or a sequence having at least 90% sequence identity to any of the foregoing. In some aspects, the gRNA further comprises a scaffold sequence comprising SEQ ID NO:44, or a sequence having at least 90% sequence identity to SEQ ID NO:44. In some aspects, the gRNA, including a spacer sequence and a scaffold sequence, comprises SEQ ID NO:49, or a sequence having at least 90% sequence identity to all or a portion thereof. In some aspects, the gRNA is set forth in SEQ ID NO:49. In some aspects, a provided DNA-targeting system comprises any of the above gRNAs complexed with a Cas protein, such as a Cas9 protein. In some aspects, the Cas9 is a dCas9. In some aspects, the dCas9 is a dSaCas9, such as a dSaCas9 set forth in SEQ ID NO:72, or a variant and/or fusion thereof.

In some aspects, the gRNA targets a target site in a FXN locus that comprises SEQ ID NO:4, a contiguous portion thereof of at least 14 nucleotides, a complementary sequence of any of the foregoing, or a sequence having at least 90% sequence identity to any of the foregoing. In some aspects, the gRNA comprises a spacer sequence comprising SEQ ID NO:25, a contiguous portion thereof of at least 14 nt, or a sequence having at least 90% sequence identity to any of the foregoing. In some aspects, the gRNA further comprises a scaffold sequence comprising SEQ ID NO:44, or a sequence having at least 90% sequence identity to SEQ ID NO:44. In some aspects, the gRNA, including a spacer sequence and a scaffold sequence, comprises SEQ ID NO:50, or a sequence having at least 90% sequence identity to all or a portion thereof. In some aspects, the gRNA is set forth in SEQ ID NO:50. In some aspects, a provided DNA-targeting system comprises any of the above gRNAs complexed with a Cas protein, such as a Cas9 protein. In some aspects, the Cas9 is a dCas9. In some aspects, the dCas9 is a dSaCas9, such as a dSaCas9 set forth in SEQ ID NO:72, or a variant and/or fusion thereof.

In some aspects, the gRNA targets a target site in a FXN locus that comprises SEQ ID NO:5, a contiguous portion thereof of at least 14 nucleotides, a complementary sequence of any of the foregoing, or a sequence having at least 90% sequence identity to any of the foregoing. In some aspects, the gRNA comprises a spacer sequence comprising SEQ ID NO:26, a contiguous portion thereof of at least 14 nt, or a sequence having at least 90% sequence identity to any of the foregoing. In some aspects, the gRNA further comprises a scaffold sequence comprising SEQ ID NO:44, or a sequence having at least 90% sequence identity to SEQ ID NO:44. In some aspects, the gRNA, including a spacer sequence and a scaffold sequence, comprises SEQ ID NO:51, or a sequence having at least 90% sequence identity to all or a portion thereof. In some aspects, the gRNA is set forth in SEQ ID NO:51. In some aspects, a provided DNA-targeting system comprises any of the above gRNAs complexed with a Cas protein, such as a Cas9 protein. In some aspects, the Cas9 is a dCas9. In some aspects, the dCas9 is a dSaCas9, such as a dSaCas9 set forth in SEQ ID NO:72, or a variant and/or fusion thereof.

In some aspects, the gRNA targets a target site in a FXN locus that comprises SEQ ID NO:6, a contiguous portion thereof of at least 14 nucleotides, a complementary sequence of any of the foregoing, or a sequence having at least 90% sequence identity to any of the foregoing. In some aspects, the gRNA comprises a spacer sequence comprising SEQ ID NO:27, a contiguous portion thereof of at least 14 nt, or a sequence having at least 90% sequence identity to any of the foregoing. In some aspects, the gRNA further comprises a scaffold sequence comprising SEQ ID NO:44, or a sequence having at least 90% sequence identity to SEQ ID NO:44. In some aspects, the gRNA, including a spacer sequence and a scaffold sequence, comprises SEQ ID NO:52, or a sequence having at least 90% sequence identity to all or a portion thereof. In some aspects, the gRNA is set forth in SEQ ID NO:52. In some aspects, a provided DNA-targeting system comprises any of the above gRNAs complexed with a Cas protein, such as a Cas9 protein. In some aspects, the Cas9 is a dCas9. In some aspects, the dCas9 is a dSaCas9, such as a dSaCas9 set forth in SEQ ID NO:72, or a variant and/or fusion thereof.

In some aspects, the gRNA targets a target site in a FXN locus that comprises SEQ ID NO:6, a contiguous portion thereof of at least 14 nucleotides, a complementary sequence of any of the foregoing, or a sequence having at least 90% sequence identity to any of the foregoing. In some aspects, the gRNA comprises a spacer sequence comprising SEQ ID NO:27, a contiguous portion thereof of at least 14 nt, or a sequence having at least 90% sequence identity to any of the foregoing. In some aspects, the gRNA further comprises a scaffold sequence comprising SEQ ID NO:44, or a sequence having at least 90% sequence identity to SEQ ID NO:44. In some aspects, the gRNA, including a spacer sequence and a scaffold sequence, comprises SEQ ID NO:52, or a sequence having at least 90% sequence identity to all or a portion thereof. In some aspects, the gRNA is set forth in SEQ ID NO:52. In some aspects, a provided DNA-targeting system comprises any of the above gRNAs complexed with a Cas protein, such as a Cas9 protein. In some aspects, the Cas9 is a dCas9. In some aspects, the dCas9 is a dSaCas9, such as a dSaCas9 set forth in SEQ ID NO:72, or a variant and/or fusion thereof.

In some aspects, the gRNA targets a target site in a FXN locus that comprises SEQ ID NO:7, a contiguous portion thereof of at least 14 nucleotides, a complementary sequence of any of the foregoing, or a sequence having at least 90% sequence identity to any of the foregoing. In some aspects, the gRNA comprises a spacer sequence comprising SEQ ID NO:28, a contiguous portion thereof of at least 14 nt, or a sequence having at least 90% sequence identity to any of the foregoing. In some aspects, the gRNA further comprises a scaffold sequence comprising SEQ ID NO:44, or a sequence having at least 90% sequence identity to SEQ ID NO:44. In some aspects, the gRNA, including a spacer sequence and a scaffold sequence, comprises SEQ ID NO:53, or a sequence having at least 90% sequence identity to all or a portion thereof. In some aspects, the gRNA is set forth in SEQ ID NO:53. In some aspects, a provided DNA-targeting system comprises any of the above gRNAs complexed with a Cas protein, such as a Cas9 protein. In some aspects, the Cas9 is a dCas9. In some aspects, the dCas9 is a dSaCas9, such as a dSaCas9 set forth in SEQ ID NO:72, or a variant and/or fusion thereof.

In some aspects, the gRNA targets a target site in a FXN locus that comprises SEQ ID NO:8, a contiguous portion thereof of at least 14 nucleotides, a complementary sequence of any of the foregoing, or a sequence having at least 90% sequence identity to any of the foregoing. In some aspects, the gRNA comprises a spacer sequence comprising SEQ ID NO:29, a contiguous portion thereof of at least 14 nt, or a sequence having at least 90% sequence identity to any of the foregoing. In some aspects, the gRNA further comprises a scaffold sequence comprising SEQ ID NO:44, or a sequence having at least 90% sequence identity to SEQ ID NO:44. In some aspects, the gRNA, including a spacer sequence and a scaffold sequence, comprises SEQ ID NO:54, or a sequence having at least 90% sequence identity to all or a portion thereof. In some aspects, the gRNA is set forth in SEQ ID NO:54. In some aspects, a provided DNA-targeting system comprises any of the above gRNAs complexed with a Cas protein, such as a Cas9 protein. In some aspects, the Cas9 is a dCas9. In some aspects, the dCas9 is a dSaCas9, such as a dSaCas9 set forth in SEQ ID NO:72, or a variant and/or fusion thereof.

In some aspects, the gRNA targets a target site in a FXN locus that comprises SEQ ID NO:8, a contiguous portion thereof of at least 14 nucleotides, a complementary sequence of any of the foregoing, or a sequence having at least 90% sequence identity to any of the foregoing. In some aspects, the gRNA comprises a spacer sequence comprising SEQ ID NO:29, a contiguous portion thereof of at least 14 nt, or a sequence having at least 90% sequence identity to any of the foregoing. In some aspects, the gRNA further comprises a scaffold sequence comprising SEQ ID NO:44, or a sequence having at least 90% sequence identity to SEQ ID NO:44. In some aspects, the gRNA, including a spacer sequence and a scaffold sequence, comprises SEQ ID NO:54, or a sequence having at least 90% sequence identity to all or a portion thereof. In some aspects, the gRNA is set forth in SEQ ID NO:54. In some aspects, a provided DNA-targeting system comprises any of the above gRNAs complexed with a Cas protein, such as a Cas9 protein. In some aspects, the Cas9 is a dCas9. In some aspects, the dCas9 is a dSaCas9, such as a dSaCas9 set forth in SEQ ID NO:72, or a variant and/or fusion thereof.

In some aspects, the gRNA targets a target site in a FXN locus that comprises SEQ ID NO:9, a contiguous portion thereof of at least 14 nucleotides, a complementary sequence of any of the foregoing, or a sequence having at least 90% sequence identity to any of the foregoing. In some aspects, the gRNA comprises a spacer sequence comprising SEQ ID NO:30, a contiguous portion thereof of at least 14 nt, or a sequence having at least 90% sequence identity to any of the foregoing. In some aspects, the gRNA further comprises a scaffold sequence comprising SEQ ID NO:44, or a sequence having at least 90% sequence identity to SEQ ID NO:44. In some aspects, the gRNA, including a spacer sequence and a scaffold sequence, comprises SEQ ID NO:55, or a sequence having at least 90% sequence identity to all or a portion thereof. In some aspects, the gRNA is set forth in SEQ ID NO:55. In some aspects, a provided DNA-targeting system comprises any of the above gRNAs complexed with a Cas protein, such as a Cas9 protein. In some aspects, the Cas9 is a dCas9. In some aspects, the dCas9 is a dSaCas9, such as a dSaCas9 set forth in SEQ ID NO:72, or a variant and/or fusion thereof.

In some aspects, the gRNA targets a target site in a FXN locus that comprises SEQ ID NO: 10, a contiguous portion thereof of at least 14 nucleotides, a complementary sequence of any of the foregoing, or a sequence having at least 90% sequence identity to any of the foregoing. In some aspects, the gRNA comprises a spacer sequence comprising SEQ ID NO:31, a contiguous portion thereof of at least 14 nt, or a sequence having at least 90% sequence identity to any of the foregoing. In some aspects, the gRNA further comprises a scaffold sequence comprising SEQ ID NO:44, or a sequence having at least 90% sequence identity to SEQ ID NO:44. In some aspects, the gRNA, including a spacer sequence and a scaffold sequence, comprises SEQ ID NO:56, or a sequence having at least 90% sequence identity to all or a portion thereof. In some aspects, the gRNA is set forth in SEQ ID NO:56. In some aspects, a provided DNA-targeting system comprises any of the above gRNAs complexed with a Cas protein, such as a Cas9 protein. In some aspects, the Cas9 is a dCas9. In some aspects, the dCas9 is a dSaCas9, such as a dSaCas9 set forth in SEQ ID NO:72, or a variant and/or fusion thereof.

In some aspects, the gRNA targets a target site in a FXN locus that comprises SEQ ID NO:21, a contiguous portion thereof of at least 14 nucleotides, a complementary sequence of any of the foregoing, or a sequence having at least 90% sequence identity to any of the foregoing. In some aspects, the gRNA comprises a spacer sequence comprising SEQ ID NO:42, a contiguous portion thereof of at least 14 nt, or a sequence having at least 90% sequence identity to any of the foregoing. In some aspects, the gRNA further comprises a scaffold sequence comprising SEQ ID NO:44, or a sequence having at least 90% sequence identity to SEQ ID NO:44. In some aspects, the gRNA, including a spacer sequence and a scaffold sequence, comprises SEQ ID NO:67, or a sequence having at least 90% sequence identity to all or a portion thereof. In some aspects, the gRNA is set forth in SEQ ID NO:67. In some aspects, a provided DNA-targeting system comprises any of the above gRNAs complexed with a Cas protein, such as a Cas9 protein. In some aspects, the Cas9 is a dCas9. In some aspects, the dCas9 is a dSaCas9, such as a dSaCas9 set forth in SEQ ID NO:72, or a variant and/or fusion thereof.

In some embodiments, the gRNA targets a target site in a FXN locus or a DNA regulatory element thereof that comprises the sequence selected from any one of SEQ ID NO:11-20, a contiguous portion thereof of at least 14 nucleotides (e.g., 14, 15, 16, 17, 18, 19, 20, 21, or 22 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising the sequence selected from any one of SEQ ID NO:32-41, a contiguous portion thereof of at least 14 nt (e.g., 14, 15, 16, 17, 18, 19, 20, 21, or 22 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO:46, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:46. In some embodiments, the gRNA, including a spacer sequence and a scaffold sequence, comprises the sequence selected from any one of SEQ ID NO:57-66, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA is set forth in the sequence selected from any one of SEQ ID NO:57-66. In some embodiments, a provided DNA-targeting system comprises any of the aforementioned gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO:78, or a variant and/or fusion thereof.

In some embodiments, the gRNA targets a target site in a FXN locus or a DNA regulatory element thereof that comprises the sequence selected from any one of SEQ ID NO:256-265, a contiguous portion thereof of at least 14 nucleotides (e.g., 14, 15, 16, 17, 18, 19, 20, 21, or 22 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO:46, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:46. In some embodiments, the gRNA comprises, in 5′ to 3′ order, a spacer targeting SEQ ID NO:256, and a scaffold sequence of SEQ ID NO:46. In some embodiments, the gRNA comprises, in 5′ to 3′ order, a spacer targeting SEQ ID NO:257, and a scaffold sequence of SEQ ID NO:46. In some embodiments, the gRNA comprises, in 5′ to 3′ order, a spacer targeting SEQ ID NO:258, and a scaffold sequence of SEQ ID NO:46. In some embodiments, the gRNA comprises, in 5′ to 3′ order, a spacer targeting SEQ ID NO:259, and a scaffold sequence of SEQ ID NO:46. In some embodiments, the gRNA comprises, in 5′ to 3′ order, a spacer targeting SEQ ID NO:260, and a scaffold sequence of SEQ ID NO:46. In some embodiments, the gRNA comprises, in 5′ to 3′ order, a spacer targeting SEQ ID NO:261, and a scaffold sequence of SEQ ID NO:46. In some embodiments, the gRNA comprises, in 5′ to 3′ order, a spacer targeting SEQ ID NO:262, and a scaffold sequence of SEQ ID NO:46. In some embodiments, the gRNA comprises, in 5′ to 3′ order, a spacer targeting SEQ ID NO:263, and a scaffold sequence of SEQ ID NO:46. In some embodiments, the gRNA comprises, in 5′ to 3′ order, a spacer targeting SEQ ID NO:264, and a scaffold sequence of SEQ ID NO:46. In some embodiments, the gRNA comprises, in 5′ to 3′ order, a spacer targeting SEQ ID NO:265, and a scaffold sequence of SEQ ID NO:46. In some embodiments, a provided DNA-targeting system comprises any of the aforementioned gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO:78, or a variant and/or fusion thereof.

In some embodiments, the gRNA targets a target site in a FXN locus that comprises SEQ ID NO:11, a contiguous portion thereof of at least 14 nucleotides, a complementary sequence of any of the foregoing, or a sequence having at least 90% sequence identity to any of the foregoing. In some embodiments, the gRNA comprises a spacer sequence comprising SEQ ID NO:32, a contiguous portion thereof of at least 14 nt, or a sequence having at least 90% sequence identity to any of the foregoing. In some embodiments, the gRNA further comprises a scaffold sequence comprising the sequence set forth in SEQ ID NO:46, or a sequence having at least 90% sequence identity to SEQ ID NO:46. In some embodiments, the gRNA, including a spacer sequence and a scaffold sequence, comprises SEQ ID NO:57, or a sequence having at least 90% sequence identity to all or a portion thereof. In some embodiments, the gRNA is set forth in SEQ ID NO:57. In some embodiments, a provided DNA-targeting system comprises any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO:78, or a variant and/or fusion thereof.

In some embodiments, the gRNA targets a target site in a FXN locus that comprises SEQ ID NO: 12, a contiguous portion thereof of at least 14 nucleotides, a complementary sequence of any of the foregoing, or a sequence having at least 90% sequence identity to any of the foregoing. In some embodiments, the gRNA comprises a spacer sequence comprising SEQ ID NO:33, a contiguous portion thereof of at least 14 nt, or a sequence having at least 90% sequence identity to any of the foregoing. In some embodiments, the gRNA further comprises a scaffold sequence comprising the sequence set forth in SEQ ID NO:46, or a sequence having at least 90% sequence identity to SEQ ID NO:46. In some embodiments, the gRNA, including a spacer sequence and a scaffold sequence, comprises SEQ ID NO:58, or a sequence having at least 90% sequence identity to all or a portion thereof. In some embodiments, the gRNA is set forth in SEQ ID NO:58. In some embodiments, a provided DNA-targeting system comprises any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO:78, or a variant and/or fusion thereof.

In some embodiments, the gRNA targets a target site in a FXN locus that comprises SEQ ID NO:13, a contiguous portion thereof of at least 14 nucleotides, a complementary sequence of any of the foregoing, or a sequence having at least 90% sequence identity to any of the foregoing. In some embodiments, the gRNA comprises a spacer sequence comprising SEQ ID NO:34, a contiguous portion thereof of at least 14 nt, or a sequence having at least 90% sequence identity to any of the foregoing. In some embodiments, the gRNA further comprises a scaffold sequence comprising the sequence set forth in SEQ ID NO:46, or a sequence having at least 90% sequence identity to SEQ ID NO:46. In some embodiments, the gRNA, including a spacer sequence and a scaffold sequence, comprises SEQ ID NO:59, or a sequence having at least 90% sequence identity to all or a portion thereof. In some embodiments, the gRNA is set forth in SEQ ID NO:59. In some embodiments, a provided DNA-targeting system comprises any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO:78, or a variant and/or fusion thereof.

In some embodiments, the gRNA targets a target site in a FXN locus that comprises SEQ ID NO:14, a contiguous portion thereof of at least 14 nucleotides, a complementary sequence of any of the foregoing, or a sequence having at least 90% sequence identity to any of the foregoing. In some embodiments, the gRNA comprises a spacer sequence comprising SEQ ID NO:35, a contiguous portion thereof of at least 14 nt, or a sequence having at least 90% sequence identity to any of the foregoing. In some embodiments, the gRNA further comprises a scaffold sequence comprising the sequence set forth in SEQ ID NO:46, or a sequence having at least 90% sequence identity to SEQ ID NO:46. In some embodiments, the gRNA, including a spacer sequence and a scaffold sequence, comprises SEQ ID NO:60, or a sequence having at least 90% sequence identity to all or a portion thereof. In some embodiments, the gRNA is set forth in SEQ ID NO:60. In some embodiments, a provided DNA-targeting system comprises any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO:78, or a variant and/or fusion thereof.

In some embodiments, the gRNA targets a target site in a FXN locus that comprises SEQ ID NO:15, a contiguous portion thereof of at least 14 nucleotides, a complementary sequence of any of the foregoing, or a sequence having at least 90% sequence identity to any of the foregoing. In some embodiments, the gRNA comprises a spacer sequence comprising SEQ ID NO:36, a contiguous portion thereof of at least 14 nt, or a sequence having at least 90% sequence identity to any of the foregoing. In some embodiments, the gRNA further comprises a scaffold sequence comprising the sequence set forth in SEQ ID NO:46, or a sequence having at least 90% sequence identity to SEQ ID NO:46. In some embodiments, the gRNA, including a spacer sequence and a scaffold sequence, comprises SEQ ID NO:61, or a sequence having at least 90% sequence identity to all or a portion thereof. In some embodiments, the gRNA is set forth in SEQ ID NO:61. In some embodiments, a provided DNA-targeting system comprises any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO:78, or a variant and/or fusion thereof.

In some embodiments, the gRNA targets a target site in a FXN locus that comprises SEQ ID NO: 16, a contiguous portion thereof of at least 14 nucleotides, a complementary sequence of any of the foregoing, or a sequence having at least 90% sequence identity to any of the foregoing. In some embodiments, the gRNA comprises a spacer sequence comprising SEQ ID NO:37, a contiguous portion thereof of at least 14 nt, or a sequence having at least 90% sequence identity to any of the foregoing. In some embodiments, the gRNA further comprises a scaffold sequence comprising the sequence set forth in SEQ ID NO:46, or a sequence having at least 90% sequence identity to SEQ ID NO:46. In some embodiments, the gRNA, including a spacer sequence and a scaffold sequence, comprises SEQ ID NO:62, or a sequence having at least 90% sequence identity to all or a portion thereof. In some embodiments, the gRNA is set forth in SEQ ID NO:62. In some embodiments, a provided DNA-targeting system comprises any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO:78, or a variant and/or fusion thereof.

In some embodiments, the gRNA targets a target site in a FXN locus that comprises SEQ ID NO:17, a contiguous portion thereof of at least 14 nucleotides, a complementary sequence of any of the foregoing, or a sequence having at least 90% sequence identity to any of the foregoing. In some embodiments, the gRNA comprises a spacer sequence comprising SEQ ID NO:38, a contiguous portion thereof of at least 14 nt, or a sequence having at least 90% sequence identity to any of the foregoing. In some embodiments, the gRNA further comprises a scaffold sequence comprising the sequence set forth in SEQ ID NO:46, or a sequence having at least 90% sequence identity to SEQ ID NO:46. In some embodiments, the gRNA, including a spacer sequence and a scaffold sequence, comprises SEQ ID NO:63, or a sequence having at least 90% sequence identity to all or a portion thereof. In some embodiments, the gRNA is set forth in SEQ ID NO:63. In some embodiments, a provided DNA-targeting system comprises any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO:78, or a variant and/or fusion thereof.

In some embodiments, the gRNA targets a target site in a FXN locus that comprises SEQ ID NO: 18, a contiguous portion thereof of at least 14 nucleotides, a complementary sequence of any of the foregoing, or a sequence having at least 90% sequence identity to any of the foregoing. In some embodiments, the gRNA comprises a spacer sequence comprising SEQ ID NO:39, a contiguous portion thereof of at least 14 nt, or a sequence having at least 90% sequence identity to any of the foregoing. In some embodiments, the gRNA further comprises a scaffold sequence comprising the sequence set forth in SEQ ID NO:46, or a sequence having at least 90% sequence identity to SEQ ID NO:46. In some embodiments, the gRNA, including a spacer sequence and a scaffold sequence, comprises SEQ ID NO:64, or a sequence having at least 90% sequence identity to all or a portion thereof. In some embodiments, the gRNA is set forth in SEQ ID NO:64. In some embodiments, a provided DNA-targeting system comprises any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO:78, or a variant and/or fusion thereof.

In some embodiments, the gRNA targets a target site in a FXN locus that comprises SEQ ID NO: 19, a contiguous portion thereof of at least 14 nucleotides, a complementary sequence of any of the foregoing, or a sequence having at least 90% sequence identity to any of the foregoing. In some embodiments, the gRNA comprises a spacer sequence comprising SEQ ID NO:40, a contiguous portion thereof of at least 14 nt, or a sequence having at least 90% sequence identity to any of the foregoing. In some embodiments, the gRNA further comprises a scaffold sequence comprising the sequence set forth in SEQ ID NO:46, or a sequence having at least 90% sequence identity to SEQ ID NO:46. In some embodiments, the gRNA, including a spacer sequence and a scaffold sequence, comprises SEQ ID NO:65, or a sequence having at least 90% sequence identity to all or a portion thereof. In some embodiments, the gRNA is set forth in SEQ ID NO:65. In some embodiments, a provided DNA-targeting system comprises any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO:78, or a variant and/or fusion thereof.

In some embodiments, the gRNA targets a target site in a FXN locus that comprises SEQ ID NO:20, a contiguous portion thereof of at least 14 nucleotides, a complementary sequence of any of the foregoing, or a sequence having at least 90% sequence identity to any of the foregoing. In some embodiments, the gRNA comprises a spacer sequence comprising SEQ ID NO:41, a contiguous portion thereof of at least 14 nt, or a sequence having at least 90% sequence identity to any of the foregoing. In some embodiments, the gRNA further comprises a scaffold sequence comprising the sequence set forth in SEQ ID NO:46, or a sequence having at least 90% sequence identity to SEQ ID NO:46. In some embodiments, the gRNA, including a spacer sequence and a scaffold sequence, comprises SEQ ID NO:66, or a sequence having at least 90% sequence identity to all or a portion thereof. In some embodiments, the gRNA is set forth in SEQ ID NO:66. In some embodiments, a provided DNA-targeting system comprises any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO:78, or a variant and/or fusion thereof.

C. Combinations of gRNAs

Also provided herein are combinations, including combinations of two or more guide RNAs (gRNAs). In some aspects, the provided combination of gRNAs include two or more gRNAs, each of which target particular regions of a frataxin (FXN) locus. In some aspects, the two or more gRNAs each comprise any of the gRNAs described herein.

Also provided herein is a combination, comprising a first gRNA comprising any of the gRNAs described herein, and one or more second gRNAs that binds to a second target site in a regulatory DNA element of a frataxin (FXN) locus. In some embodiments, the second gRNA comprises any of the gRNAs described herein.

Also provided herein is a combination, comprising: a first gRNA that binds a first target site in an enhancer region of a frataxin (FXN) locus, wherein the first target site is located within the genomic coordinates human genome assembly GRCh38 (hg38) chr9:69,027,282-69,028,497; and a second gRNA that binds a second target site in a promoter region of a FXN locus, wherein the second target site is located within the genomic coordinates hg38 chr9:68,940,179-69,205,519.

In some embodiments, the first gRNA comprises a gRNA spacer sequence set forth in SEQ ID NO:42 or a contiguous portion thereof of at least 14 nt. In some embodiments, the second gRNA comprises a gRNA spacer sequence set forth in any one of SEQ ID NO:22-31 or a contiguous portion thereof of at least 14 nt. In some embodiments, the second gRNA comprises a gRNA spacer sequence set forth in SEQ ID NO:22 or 28 or a contiguous portion thereof of at least 14 nt.

In some of any of the provided embodiments, e.g., of a combination provided herein: the first gRNA comprises a gRNA spacer sequence set forth in SEQ ID NO:42 or a contiguous portion thereof of at least 14 nt; and the second gRNA comprises a gRNA spacer sequence set forth in any one of SEQ ID NO:22-31 or a contiguous portion thereof of at least 14 nt. In some of any of the provided embodiments, e.g., of a combination provided herein: the first gRNA comprises a gRNA spacer sequence set forth in SEQ ID NO:42 or a contiguous portion thereof of at least 14 nt; and the second gRNA comprises a gRNA spacer sequence set forth in SEQ ID NO:22 or a contiguous portion thereof of at least 14 nt. In some of any of the provided embodiments, e.g., of a combination provided herein: the first gRNA comprises a gRNA spacer sequence set forth in SEQ ID NO:42 or a contiguous portion thereof of at least 14 nt; and the second gRNA comprises a gRNA spacer sequence set forth in SEQ ID NO:28 or a contiguous portion thereof of at least 14 nt.

Also provided herein is a combination, comprising: a first gRNA that binds a first target site in a promoter region of a FXN locus, wherein the second target site is located within the genomic coordinates hg38 chr9:68,940,179-69,205,519; and a second gRNA that binds a second target site in a promoter region of a frataxin (FXN) locus, wherein the first target site is located within the genomic coordinates hg38 chr9:68,940,179-69,205,519.

In some embodiments, the first gRNA comprises a gRNA spacer sequence set forth in any one of SEQ ID NO:22-31 or a contiguous portion thereof of at least 14 nt. In some embodiments, the first gRNA comprises a gRNA spacer sequence set forth in SEQ ID NO:22 or 28 or a contiguous portion thereof of at least 14 nt. In some embodiments, the second gRNA comprises a gRNA spacer sequence set forth in any one of SEQ ID NO:22-31 or a contiguous portion thereof of at least 14 nt. In some embodiments, the second gRNA comprises a gRNA spacer sequence set forth in SEQ ID NO:22 or 28 or a contiguous portion thereof of at least 14 nt.

In some embodiments, the combination comprises: the first gRNA comprises a gRNA spacer sequence set forth in any one of SEQ ID NO:22-31 or a contiguous portion thereof of at least 14 nt; and the second gRNA comprises a gRNA spacer sequence set forth in any one of SEQ ID NO:22-31 or a contiguous portion thereof of at least 14 nt.

In some embodiments, the combination comprises: the first gRNA comprises a gRNA spacer sequence set forth in SEQ ID NO:22 or a contiguous portion thereof of at least 14 nt; and the second gRNA comprises a gRNA spacer sequence set forth in SEQ ID NO:28 or a contiguous portion thereof of at least 14 nt.

In some embodiments, the first gRNA comprises a gRNA spacer sequence set forth in any one of SEQ ID NO: 11-20 or a contiguous portion thereof of at least 14 nt. In some embodiments, the second gRNA comprises a gRNA spacer sequence set forth in any one of SEQ ID NO:11-20 or a contiguous portion thereof of at least 14 nt. In some embodiments, the combination comprises: the first gRNA comprises a gRNA spacer sequence set forth in any one of SEQ ID NO: 11-20 or a contiguous portion thereof of at least 14 nt; and the second gRNA comprises a gRNA spacer sequence set forth in any one of SEQ ID NO:11-20 or a contiguous portion thereof of at least 14 nt.

### D. DNA Targeting Domains

In some embodiments, the provided DNA-targeting systems or fusion proteins comprise a DNA-targeting domain. In some aspects, the DNA-targeting domain provides sequence specificity and targets the DNA targeting system or fusion protein at a particular location of the genome, such as a target site specified by a component of the DNA-targeting domain. In some embodiments, exemplary DNA-targeting domain comprises a Clustered Regularly Interspaced Short Palindromic Repeats associated (Cas)-guide RNA (gRNA) combination that includes (a) a Cas protein or a variant thereof and (b) at least one gRNA; a zinc finger protein (ZFP); a transcription activator-like effector (TALE); a meganuclease; a homing endonuclease; or a I-SceI enzymes or a variant of any of the foregoing. In some embodiments, the DNA-targeting domain comprises a catalytically inactive variant of any of the foregoing. In some embodiments, the DNA-targeting domain comprises a Cas-gRNA combination that includes (a) a Cas protein or a variant thereof and (b) at least one gRNA. In some embodiments, the variant Cas protein lacks nuclease activity or is a deactivated Cas (dCas) protein. In some aspects, for a DNA-targeting domain that comprises a Cas-gRNA combination, the gRNA component (such as any described herein, for example, in Section II.B) provides the sequence specificity to target the DNA-targeting system, DNA-targeting domain or fusion protein to a target site specified by the gRNA.

### 1. Cas and Variants

In some embodiments, the DNA-targeting systems comprise a DNA-targeting domain that binds to a target site in a regulatory DNA element of a FXN locus and comprises a Cas-guide RNA (gRNA) combination. In some embodiments, the Cas-gRNA combination includes a variant Cas protein that lacks nuclease activity or that is a deactivated Cas (dCas) protein. In some embodiments, the Cas-gRNA combination includes at least one gRNA comprising a gRNA spacer sequence that is capable of hybridizing to the target site or is complementary to the target site.

In some aspects, the DNA-targeting domain comprises a CRISPR-associated (Cas) protein or variant thereof, or comprises a protein that is derived from a Cas protein or variant thereof. In particular embodiments here, the Cas protein is nuclease-inactive (i.e. is a dCas protein).

In some aspects, provided herein are DNA-targeting systems based on CRISPR/Cas systems, i.e. CRISPR/Cas-based DNA-targeting systems, that are able to bind to a target site in a FXN gene or regulatory DNA element thereof. In some embodiments, the CRISPR/Cas DNA-targeting domain is nuclease inactive, such as includes a dCas (e.g. dCas9) so that the system binds to the target site in a target gene without mediating nucleic acid cleavage at the target site. The CRISPR/Cas-based DNA-targeting systems may be used to modulate expression of FXN in a cell. In some embodiments, the CRISPR/Cas-based DNA-targeting system can include any known Cas enzyme, such as a nuclease-inactive or dCas. In some embodiments, the CRISPR/Cas-based DNA-targeting system includes a fusion protein of a nuclease-inactive Cas protein or a variant thereof and an effector domain that increases transcription of a gene (e.g. a transcription activation domain), and at least one gRNA.

The CRISPR system (also known as CRISPR/Cas system, or CRISPR-Cas system) refers to a conserved microbial nuclease system, found in the genomes of bacteria and archaea, that provides a form of acquired immunity against invading phages and plasmids. Clustered Regularly Interspaced Short Palindromic Repeats (CRISPR) refers to loci containing multiple repeating DNA elements that are separated by non-repeating DNA sequences called spacers. Spacers are short sequences of foreign DNA that are incorporated into the genome between CRISPR repeats, serving as a ‘memory’ of past exposures. Spacers encode the DNA-targeting portion of RNA molecules that confer specificity for nucleic acid cleavage by the CRISPR system. CRISPR loci contain or are adjacent to one or more CRISPR-associated (Cas) genes, which can act as RNA-guided nucleases for mediating the cleavage, as well as non-protein coding DNA elements that encode RNA molecules capable of programming the specificity of the CRISPR-mediated nucleic acid cleavage.

CRISPR/Cas systems, such as those with Cas9, have been engineered to allow efficient programming of Cas/RNA RNPs to target desired sequences in cells of interest, both for gene-editing and modulation of gene expression. The tracrRNA and crRNA have been engineered to form a single chimeric guide RNA molecule, commonly referred to as a guide RNA (gRNA), for example as described in WO 2013/176772, WO 2014/093661, WO 2014/093655, Jinek et al. Science 337(6096):816-21 (2012), or Cong et al. Science 339(6121):819-23 (2013), and as described herein, for example, in Section II.B. The spacer sequence of the gRNA can be chosen by a user to target the Cas/gRNA RNP complex to a desired locus, e.g. a desired target site in the target gene, e.g., FXN. CRISPR/Cas systems may be multi-protein systems or single effector protein systems. Multi-protein, or Class 1, CRISPR systems include Type I, Type III, and Type IV systems. In some aspects, Class 2 systems include a single effector molecule and include Type II, Type V, and Type VI. In some embodiments, the DNA targeting system comprises components of CRISPR/Cas systems, such as a Type I, Type II, Type III, Type IV, Type V, or Type VI CRISPR system. In some embodiments, the Cas protein is from a Class 1 CRISPR system (i.e. multiple Cas protein system), such as a Type I, Type III, or Type IV CRISPR system. In some embodiments, the Cas protein is from a Class 2 CRISPR system (i.e. single Cas protein system), such as a Type II, Type V, or Type VI CRISPR system.

In some embodiments, the Cas protein is derived from a Cas9 protein or variant thereof, for example as described in WO 2013/176772, WO 2014/152432, WO 2014/093661, WO 2014/093655, Jinek, M. et al. Science 337(6096): 816-21 (2012), Mali, P. et al. Science 339(6121):823-6 (2013), Cong. L. et al. Science 339(6121):819-23 (2013), Perez-Pincra, P. et al. Nat. Methods 10, 973-976 (2013), or Mali, P. et al. Nat. Biotechnol. 31, 833-838 (2013). Various CRISPR/Cas systems and associated Cas proteins for use in gene editing and regulation have been described, for example in Moon et al. Exp. Mol. Med. 51, 1-11 (2019), Zhang, F. Q. Rev. Biophys. 52, E6 (2019), and Makarova et al. Methods Mol. Biol. 1311:47-75 (2015).

Type I CRISPR/Cas systems employ a large multisubunit ribonucleoprotein (RNP) complex called Cascade that recognizes double-stranded DNA (dsDNA) targets. After target recognition and verification, Cascade recruits the signature protein Cas3, a fused helicase-nuclease, to degrade DNA.

In some embodiments, the Cas protein is from a Type II CRISPR system. Exemplary Cas proteins of a Type II CRISPR system include Cas9. In some embodiments, the Cas protein is from a Cas9 protein or variant thereof, for example as described in WO 2013/176772, WO 2014/152432, WO 2014/093661, WO 2014/093655, Jinek. et al. Science 337(6096):816-21 (2012), Mali et al. Science 339(6121):823-6 (2013), Cong et al. Science 339(6121):819-23 (2013), Perez-Pinera et al. Nat. Methods 10, 973-976 (2013), or Mali et al. Nat. Biotechnol. 31, 833-838 (2013). In Type II CRISPR/Cas systems with the Cas protein Cas9, two RNA molecules and the Cas9 protein form a ribonucleoprotein (RNP) complex to direct Cas9 nuclease activity. The CRISPR RNA (crRNA) contains a spacer sequence that is complementary to a target nucleic acid sequence (target site), and that encodes the sequence specificity of the complex. The trans-activating crRNA (tracrRNA) base-pairs to a portion of the crRNA and forms a structure that complexes with the Cas9 protein, forming a Cas/RNA RNP complex. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage.

Different Type II systems have differing PAM requirements. The S. pyogenes CRISPR system may have the PAM sequence for this Cas9 (SpCas9) as 5′-NRG-3′, where R is either A or G, and characterized the specificity of this system in human cells. A unique capability of the CRISPR/Cas9 system is the straightforward ability to simultaneously target multiple distinct genomic loci by co-expressing a single Cas9 protein with two or more sgRNAs. For example, the Streptococcus pyogenes Type II system typically prefers to use an “NGG” sequence, where “N” can be any nucleotide, but also accepts other PAM sequences, such as “NAG” in engineered systems (Hsu et al., Nature Biotechnology (2013) doi: 10.1038/nbt.2647). Similarly, the Cas9 derived from Neisseria meningitidis (NmCas9) normally has a native PAM of NNNNGATT (SEQ ID NO:143), but has activity across a variety of PAMs, including a highly degenerate NNNNGNNN (SEQ ID NO:222) PAM (Esvelt et al. Nature Methods (2013) doi: 10.1038/nmeth.2681). In another example, the Cas9 derived from Campylobacter jejuni typically uses 5′-NNNNACAC-3′ (SEQ ID NO:226) or 5′-NNNNRYAC-3′ (SEQ ID NO: 144) PAM sequences, where “N” can be any nucleotide, “R” can be either guanine (G) or adenine (A), and “Y” can be either cytosine (C) or thymine (T). In some aspects, the PAM sequences for spacer targeting depends on the type, ortholog, variant or species of the Cas protein.

In some embodiments, the Cas9 protein comprises a sequence from a Cas9 molecule of S. aureus. In some embodiments, the Cas9 protein comprises a sequence set forth in SEQ ID NO:73 or SEQ ID NO:126, or a variant thereof, such as an amino acid sequence that has at least 90%, 91%, 92%, 93%, 94%, 95%, 96%, 97%, 98%, or 99% sequence identity to SEQ ID NO:73 or SEQ ID NO: 126. In some embodiments, the Cas9 protein comprises a sequence from a Cas9 molecule of S. pyogenes. In some embodiments, the Cas9 protein comprises a sequence set forth in SEQ ID NO:79 or SEQ ID NO:127, or a variant thereof, such as an amino acid sequence that has at least 90%, 91%, 92%, 93%, 94%, 95%, 96%, 97%, 98%, or 99% sequence identity to SEQ ID NO:79 or SEQ ID NO:127.

In Type III systems, the RNP complex is multimeric with a helicoid structure similar to Cascade. In contrast to Type I CRISPR/Cas systems, the Type III RNP complex recognizes complementary RNA sequences instead of dsDNA. RNA recognition stimulates a nonspecific DNA cleavage activity of the exemplary Type III Cas10 nuclease that is part of the RNP complex, such that DNA cleavage is achieved cotranscriptionally.

In some embodiments, the Cas protein is from a Type V CRISPR system. Exemplary Cas proteins of a Type V CRISPR system include Cas12a (also known as Cpf1), Cas12b (also known as C2c1), Cas12e (also known as CasX), Cas12k (also known as C2c5), Cas14a, and Cas14b. In some embodiments, the Cas protein is from a Cas12 protein (i.e. Cpf1) or variant thereof, for example as described in WO 2017/189308, WO2019/232069 and Zetsche et al. Cell. 163(3):759-71 (2015).

Exemplary Type V systems include those based on a Cas12 effector, and the C-terminus with only one RuvC endonuclease domain is the defining characteristic of the Type V systems. The RuvC nuclease domain cleaves dsDNA adjacent to protospacer adjacent motif (PAM) sequences and single-stranded DNA (ssDNA) nonspecifically. The Type V systems can be further divided into subtypes, each characterized by different signature proteins, PAM sequences, and properties. Non-limiting exemplary Cas proteins derived from Type V CRISPR systems include Cas12a (Cpf1), Un1Cas12f1, Cas12j (CasPhi, such as CasPhi-2), Cas12k, and CasMini. For example, Type V-A includes, for example, Cas12a, which uses “TTTV” (SEQ ID NO:147) PAM sequence, where “V” is adenine (A), cytosine (C), or guanine (G). Type V-F is includes, for example, Cas12f, which can use “TTTR” (SEQ ID NO:228), where “R” is G or A, or “TTTN” (SEQ ID NO:225), where “N” is any nucleotide. Type V-K is includes, for example, Cas12k, which uses “GGTT” (SEQ ID NO:227) PAM sequence.

In some embodiments, the Cas12a protein comprises a sequence from a Cas12a molecule of Acidaminococcus sp, such as an AsCas12a set forth in SEQ ID NO:191 or SEQ ID NO:192, or a variant thereof, such as an amino acid sequence that has at least 90%, 91%, 92%, 93%, 94%, 95%, 96%, 97%, 98%, or 99% sequence identity to SEQ ID NO: 191 or SEQ ID NO:192.

Non-limiting examples of Cas proteins or Cas orthologs, such as Cas9 orthologs, from other bacterial strains include but are not limited to, Cas proteins identified in Acaryochloris marina MBIC11017; Acctohalobium arabaticum DSM 5501; Acidaminococcus sp.; Acidithiobacillus caldus; Acidithiobacillus ferrooxidans ATCC 23270; Alicyclobacillus acidocaldarius LAA1; Alicyclobacillus acidocaldarius subsp. acidocaldarius DSM 446; Allochromatium vinosum DSM 180; Ammonifex degensii KC4; Anabaena variabilis ATCC 29413; Arthrospira maxima CS-328; Arthrospira platensis str. Paraca; Arthrospira sp. PCC 8005; Bacillus pseudomycoides DSM 12442; Bacillus selenitireducens MLS10; Burkholderiales bacterium 1_1_47; Caldicelulosiruptor becscii DSM 6725; Campylobacter jejuni; Candidatus Desulforudis audaxviator MP104C; Caldicellulosiruptor hydrothermalis 108; Clostridium phage c-st; Clostridium botulinum A3 str. Loch Marce; Clostridium botulinum Ba4 str. 657; Clostridium difficile QCD-63q42; Crocosphaera watsonii WH 8501; Cyanothece sp. ATCC 51142; Cyanothece sp. CCY0110; Cyanothece sp. PCC 7424; Cyanothece sp. PCC 7822; Exiguobacterium sibiricum 255-15; Finegoldia magna ATCC 29328; Ktedonobacter racemifer DSM 44963; Lactobacillus delbrueckii subsp. bulgaricus PB2003/044-T3-4; Lactobacillus salivarius ATCC 11741; Listeria innocua; Lyngbya sp. PCC 8106; Marinobacter sp. ELB17; Methanohalobium evestigatum Z-7303; Microcystis phage Ma-LMM01; Microcystis acruginosa NIES-843; Microscilla marina ATCC 23134; Microcoleus chthonoplastes PCC 7420; Neisseria meningitidis; Nitrosococcus halophilus Nc4; Nocardiopsis dassonvillei subsp. dassonvillei DSM 43111; Nodularia spumigena CCY9414; Nostoc sp. PCC 7120; Oscillatoria sp. PCC 6506; Pelotomaculum thermopropionicum SI; Petrotoga mobilis SJ95; Polaromonas naphthalenivorans CJ2; Polaromonas sp. JS666; Pseudoalteromonas haloplanktis TAC125; Streptomyces pristinaespiralis ATCC 25486; Streptomyces pristinaespiralis ATCC 25486; Streptococcus thermophilus; Streptomyces viridochromogenes DSM 40736; Streptosporangium roscum DSM 43021; Synechococcus sp. PCC 7335; and Thermosipho africanus TCF52B (Chylinski et al., RNA Biol., 2013; 10(5): 726-737).

In some embodiments, the DNA-targeting systems or fusion proteins comprise a Cas protein, such as a Cas protein set forth in any one of SEQ ID NOS:73, 79, 126, 127, 193, 194, 197-200, and 205-208, or a variant thereof, such as an amino acid sequence that has at least 90%, 91%, 92%, 93%, 94%, 95%, 96%, 97%, 98%, or 99% sequence identity to any one of SEQ ID NOS:73, 79, 126, 127, 193, 194, 197-200, and 205-208. In some embodiments, the Cas protein of any of the DNA-targeting systems or fusion proteins provided herein comprise a sequence set forth in any one of SEQ ID NOS:73, 79, 126, 127, 193, 194, 197-200, and 205-208, or a variant thereof, such as an amino acid sequence that has at least 90%, 91%, 92%, 93%, 94%, 95%, 96%, 97%, 98%, or 99% sequence identity to any one of SEQ ID NOS:73, 79, 126, 127, 193, 194, 197-200, and 205-208. In some aspects, the Cas protein lacks an initial methionine residue. In some aspects, the Cas protein comprises an initial methionine residue.

In some aspects, in the provided DNA-targeting systems and fusion proteins, the DNA-targeting domain, e.g., Cas, is a deactivated Cas (dCas), or a nuclease-inactive Cas (iCas). In some embodiments, the component of the DNA-targeting domain, such as a protein component, comprises a Cas9 variant such as a deactivated Cas9 or inactivated Cas9. In some embodiments, the component of the DNA-targeting domain, such as a protein component, comprises a Cas12a variant such as a deactivated Cas12a (Cpf1) or inactivated Cas12a (Cpf1). In some aspects, the Cas9 protein may be mutated so that the nuclease activity is deactivated or inactivated (also referred to as dCas9 or iCas9). In some aspects, the Cas protein is a variant that lacks nuclease activity (i.e. is a dCas or iCas protein). In some embodiments, the Cas protein is mutated so that nuclease activity is reduced or eliminated. Such Cas proteins are referred to as deactivated Cas or dead Cas (dCas) or nuclease-inactive Cas (iCas) proteins, as referred to interchangeably herein. In some embodiments, the variant Cas protein is a variant Cas9 protein that lacks nuclease activity or that is a deactivated Cas9 (dCas9, or iCas9) protein. In some embodiments, the variant Cas protein is a variant Cpf1 protein that lacks nuclease activity or that is a deactivated Cas12a (dCas12a, or iCas12a) protein.

In some embodiments, Cas proteins are engineered to be catalytically inactivated or nuclease inactive to allow targeting of Cas/gRNA RNPs without inducing cleavage at the target site. Mutations in Cas proteins can reduce or abolish nuclease activity of the Cas protein, rendering the Cas protein catalytically inactive. Cas proteins with reduced or abolished nuclease activity are referred to as deactivated Cas (dCas), or nuclease-inactive Cas (iCas) proteins, as referred to interchangeably herein. In some aspects, the dCas or iCas can still bind to target site in the DNA in a site- and/or sequence-specific manner, as long as it retains the ability to interact with the guide RNA (gRNA) which directs the Cas-gRNA combination to the target site.

In some aspects, the dCas or iCas exhibits reduced or no endodeoxyribonuclease activity. For example, an exemplary dCas or iCas, for example dCas9 or iCas9, exhibits less than about 20%, less than about 15%, less than about 10%, less than about 5%, less than about 1%, or less than about 0.1%, of the endodeoxyribonuclease activity of a wild-type Cas protein, e.g., a wild-type Cas9 protein. In some embodiments, the dCas or iCas, for example dCas9 or iCas9, exhibits substantially no detectable endodeoxyribonuclease activity. In some embodiments, an exemplary dCas or iCas, for example dCas9 or iCas9, comprises one or more amino acid mutations, substitutions, deletions or insertions at a position corresponding to a position selected from D10, G12, G17, E762, H840, N854, N863, H982, H983, A984, D986, and/or a A987, with reference to a wild-type Streptococcus pyogenes Cas9 (SpCas9), for example, with reference to numbering of positions of a SpCas9 sequence set forth in SEQ ID NO:79. In some aspects, the dCas9 or iCas9 comprises one or more amino acid mutations, substitutions, deletions or insertions corresponding to D10A, G12A, G17A, E762A, H840A, N854A, N863A, H982A, H983A, A984A, and/or D986A, with reference to a wild-type Streptococcus pyogenes Cas9 (SpCas9), for example, with reference to numbering of positions of a SpCas9 sequence set forth in SEQ ID NO:79. Corresponding positions for mutations can be determined based on sequence alignments and determination of sequence conservation, for example, as described in WO 2013/171772 for Cas9 proteins from various species. In some aspects, the dCas protein lacks an initial methionine residue. In some aspects, the dCas protein comprises an initial methionine residue.

In some embodiments, the dCas9 protein can comprise a sequence from a Cas9 molecule, or variant thereof. In some embodiments, the dCas9 protein can comprise a sequence derived from a Cas9 molecule of S. pyogenes, S. thermophilus, S. aureus, N. meningitidis, F. novicida, S. canis, S. auricularis, or variant thereof. In some embodiments, the dCas9 protein comprises a sequence from a Cas9 molecule of S. aureus. In some embodiments, the dCas9 protein comprises a sequence from a Cas9 molecule of S. pyogenes. In some embodiments, the dCas9 protein comprises a sequence from a Cas9 molecule of C. jejuni.

Exemplary deactivated Cas9 (dCas9) derived from S. pyogenes contains silencing mutations of the RuvC and HNH nuclease domains (D10A and H840A), for example as described in WO 2013/176772, WO 2014/093661, Jinek et al. Science 337(6096):816-21 (2012), and Qi et al. Cell 152(5): 1173-83 (2013). Exemplary dCas variants derived from the Cas12 system (i.e. Cpf1) are described, for example in WO 2017/189308 and Zetsche et al. Cell 163(3):759-71 (2015). Conserved domains that mediate nucleic acid cleavage, such as RuvC and HNH endonuclease domains, are readily identifiable in Cas orthologs, and can be mutated to produce inactive variants, for example as described in Zetsche et al. Cell 163(3):759-71 (2015). Other exemplary Cas orthologs or variants include engineered variants based on a Cas12f (also known as Cas14), including those described in Xu et al., Mol. Cell 81(20):4333-4345 (2021).

In some embodiments, the DNA-targeting domain comprises a Cas-gRNA combination that includes (a) a Cas protein or a variant thereof and (b) at least one gRNA. In some embodiments, the variant Cas protein lacks nuclease activity or is a deactivated Cas (dCas) protein. In some embodiments, the gRNA is capable of complexing with the Cas protein or variant thereof. In some embodiments, the gRNA comprises a gRNA spacer sequence that is capable of hybridizing to the target site or is complementary to the target site (e.g., in a FXN locus).

In some embodiments, the Cas protein or a variant thereof is a Cas9 protein or a variant thereof. In some embodiments, the variant Cas protein is a variant Cas9 protein that lacks nuclease activity or that is a deactivated Cas9 (dCas9) protein. In some embodiments, the Cas9 protein or a variant thereof is a Staphylococcus aureus Cas9 (SaCas9) protein or a variant thereof. In some embodiments, the variant Cas9 is a Staphylococcus aureus dCas9 protein (dSaCas9) that comprises at least one amino acid mutation selected from D10A and N580A, with reference to numbering of positions of SEQ ID NO:73. In some embodiments, the variant Cas9 protein comprises the sequence set forth in SEQ ID NO:72, or an amino acid sequence that has at least 90%, 91%, 92%, 93%, 94%, 95%, 96%, 97%, 98%, or 99% sequence identity thereto. In some embodiments, the variant Cas9 protein comprises the sequence set forth in SEQ ID NO:72, which lacks an initial methionine residue. In some embodiments, the variant Cas9 protein comprises the sequence set forth in SEQ ID NO:189, which includes an initial methionine residue.

In some embodiments, the Cas9 protein or variant thereof is a Streptococcus pyogenes Cas9 (SpCas9) protein or a variant thereof. In some embodiments, the variant Cas9 is a Streptococcus pyogenes dCas9 (dSpCas9) protein that comprises at least one amino acid mutation selected from D10A and H840A, with reference to numbering of positions of SEQ ID NO:79. In some embodiments, the variant Cas9 protein comprises the sequence set forth in SEQ ID NO:78, or an amino acid sequence that has at least 90%, 91%, 92%, 93%, 94%, 95%, 96%, 97%, 98%, or 99% sequence identity thereto. In some embodiments, the variant Cas9 protein comprises the sequence set forth in SEQ ID NO:78, which lacks an initial methionine residue. In some embodiments, the variant Cas9 protein comprises the sequence set forth in SEQ ID NO:190, which includes an initial methionine residue.

In some embodiments, the Cas9 protein or variant thereof is a Campylobacter jejuni Cas9 (CjCas9) protein or a variant thereof. In some embodiments, the variant Cas9 comprises at least one amino acid mutation compared to the sequence set forth in SEQ ID NO:205 or 206. In some embodiments, the variant Cas9 protein comprises the sequence set forth in SEQ ID NO:203, or an amino acid sequence that has at least 90%, 91%, 92%, 93%, 94%, 95%, 96%, 97%, 98%, or 99% sequence identity thereto. In some embodiments, the variant Cas9 protein comprises the sequence set forth in SEQ ID NO:204, or an amino acid sequence that has at least 90%, 91%, 92%, 93%, 94%, 95%, 96%, 97%, 98%, or 99% sequence identity thereto. In some embodiments, the variant Cas9 protein comprises the sequence set forth in SEQ ID NO:204, which lacks an initial methionine residue. In some embodiments, the variant Cas9 protein comprises the sequence set forth in SEQ ID NO:203, which includes an initial methionine residue.

In some embodiments, the Cas protein or a variant thereof is a Cas12a protein or a variant thereof. In some embodiments, the variant Cas protein is a variant Cas12a protein that lacks nuclease activity or that is a deactivated Cas12a (dCas12a) protein. In some embodiments, the Cas12a protein or variant thereof is a Acidaminococcus sp. Cas12a (AsCas12a) protein or a variant thereof. In some embodiments, the variant Cas12a is a Acidaminococcus sp. dCas12a (dAsCas12a) protein that comprises at least one amino acid mutation compared to the sequence set forth in SEQ ID NO:193 or 194. In some embodiments, the variant Cas12a protein comprises the sequence set forth in SEQ ID NO: 191, or an amino acid sequence that has at least 90%, 91%, 92%, 93%, 94%, 95%, 96%, 97%, 98%, or 99% sequence identity thereto. In some embodiments, the variant Cas12a protein comprises the sequence set forth in SEQ ID NO:192, or an amino acid sequence that has at least 90%, 91%, 92%, 93%, 94%, 95%, 96%, 97%, 98%, or 99% sequence identity thereto. In some embodiments, the variant Cas12a protein comprises the sequence set forth in SEQ ID NO: 192, which lacks an initial methionine residue. In some embodiments, the variant Cas12a protein comprises the sequence set forth in SEQ ID NO:191, which includes an initial methionine residue.

In some embodiments, the Cas protein or a variant thereof is a CasPhi-2 protein or a variant thereof. In some embodiments, the variant Cas protein is a variant CasPhi-2 protein that lacks nuclease activity or that is a deactivated CasPhi-2 (dCasPhi-2) protein. In some embodiments, the variant CasPhi-2 comprises at least one amino acid mutation compared to the sequence set forth in SEQ ID NO:197 or 198. In some embodiments, the variant CasPhi-2 protein comprises the sequence set forth in SEQ ID NO:195, or an amino acid sequence that has at least 90%, 91%, 92%, 93%, 94%, 95%, 96%, 97%, 98%, or 99% sequence identity thereto. In some embodiments, the variant CasPhi-2 protein comprises the sequence set forth in SEQ ID NO:196, or an amino acid sequence that has at least 90%, 91%, 92%, 93%, 94%, 95%, 96%, 97%, 98%, or 99% sequence identity thereto. In some embodiments, the variant CasPhi-2 protein comprises the sequence set forth in SEQ ID NO:196, which lacks an initial methionine residue. In some embodiments, the variant CasPhi-2 protein comprises the sequence set forth in SEQ ID NO: 195, which includes an initial methionine residue.

In some embodiments, the Cas protein or a variant thereof is a Un1Cas12f1 protein or a variant thereof. In some embodiments, the variant Cas protein is a variant Un1Cas12f1 protein that lacks nuclease activity or that is a deactivated Un1Cas12f1 (dUn1Cas12f1) protein. In some embodiments, the variant Un1Cas12f1 comprises at least one amino acid mutation compared to the sequence set forth in SEQ ID NO: 199 or 200. In some embodiments, the variant Un1Cas12f1 protein comprises the sequence set forth in SEQ ID NO:201, or an amino acid sequence that has at least 90%, 91%, 92%, 93%, 94%, 95%, 96%, 97%, 98%, or 99% sequence identity thereto. In some embodiments, the variant Un1Cas12f1 protein comprises the sequence set forth in SEQ ID NO:202, or an amino acid sequence that has at least 90%, 91%, 92%, 93%, 94%, 95%, 96%, 97%, 98%, or 99% sequence identity thereto. In some embodiments, the variant Un1Cas12f1 protein comprises the sequence set forth in SEQ ID NO:202, which lacks an initial methionine residue. In some embodiments, the variant Un1Cas12f1 protein comprises the sequence set forth in SEQ ID NO:201, which includes an initial methionine residue.

In some embodiments, the Cas protein or a variant thereof is a Cas12k protein or a variant thereof. In some embodiments, the Cas12k protein comprises the sequence set forth in SEQ ID NO:207, or an amino acid sequence that has at least 90%, 91%, 92%, 93%, 94%, 95%, 96%, 97%, 98%, or 99% sequence identity thereto. In some embodiments, the Cas12k protein comprises the sequence set forth in SEQ ID NO:208, or an amino acid sequence that has at least 90%, 91%, 92%, 93%, 94%, 95%, 96%, 97%, 98%, or 99% sequence identity thereto. In some embodiments, the Cas12k protein comprises the sequence set forth in SEQ ID NO:208, which lacks an initial methionine residue. In some embodiments, the Cas12k protein comprises the sequence set forth in SEQ ID NO:207, which includes an initial methionine residue.

In some embodiments, the Cas protein or a variant thereof is a CasMini protein or a variant thereof, such as an engineered Cas protein or variant based on a Cas12f (also known as Cas14), including those described in Xu et al., Mol. Cell 81(20):4333-4345 (2021) or set forth in SEQ ID NO:223. In some embodiments, the variant Cas protein is a variant CasMini protein that lacks nuclease activity or that is a deactivated CasMini (dCasMini) protein. In some embodiments, the variant CasMini comprises at least one amino acid mutation compared to the sequence set forth in SEQ ID NO:223. In some embodiments, the variant CasMini protein comprises the sequence set forth in SEQ ID NO:223, or an amino acid sequence that has at least 90%, 91%, 92%, 93%, 94%, 95%, 96%, 97%, 98%, or 99% sequence identity thereto. In some embodiments, the CasMini protein comprises the sequence set forth in SEQ ID NO:223. In some embodiments, the variant CasMini protein comprises the sequence set forth in SEQ ID NO:209 or 210, or an amino acid sequence that has at least 90%, 91%, 92%, 93%, 94%, 95%, 96%, 97%, 98%, or 99% sequence identity thereto. In some embodiments, the CasMini protein comprises the sequence set forth in SEQ ID NO:209, which lacks an initial methionine residue. In some embodiments, the CasMini protein comprises the sequence set forth in SEQ ID NO:210, which includes an initial methionine residue.

DNA-targeting systems, in some cases comprising a fusion protein, such as dCas-fusion proteins include fusion of the Cas with an effector domain, such as a transcription activation domain. Any of a variety of effector domains, for example those that increase transcription from the target locus, e.g., FXN locus, including any described herein, for example, in Section II.D, can be used.

In some aspects, provided is a DNA-targeting system comprising a fusion protein comprising a DNA-targeting domain comprising a nuclease-inactive Cas protein or variant thereof, and an effector domain for increasing or inducing transcriptional activation (i.e. a transcriptional activator) when targeted to a target site in a FXN gene or regulatory element thereof. In some aspects, the DNA-targeting system also includes one or more gRNA, provided in combination or as a complex with the dCas protein or variant thereof, for targeting of the DNA-targeting system to the target site. In some embodiments, the fusion protein is guided to a specific target site sequence of the target gene by the guide RNA, wherein the effector domain mediates targeted epigenetic modification to increase or promote transcription of the target gene.

### 2. Other Domains

In some of any of the provided embodiments, the DNA-targeting domain comprises a zinc finger protein (ZFP); a transcription activator-like effector (TALE); a meganuclease; a homing endonuclease; or an I-SceI enzyme or a variant thereof. In some embodiments, the DNA-targeting domain comprises a catalytically inactive variant of any of the foregoing. In some aspects, types of DNA-targeting domains include domains from proteins that can recognize nucleic acid sequences (e.g., target site) in a sequence-specific manner.

In some embodiments, a “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Among the ZFPs are artificial, or engineered, ZFPs, comprising ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3, and 6) on a zinc finger recognition helix. Thus, for example, the ZFP or ZFP-containing molecule is non-naturally occurring, e.g., is engineered to bind to a target site of choice.

In some cases, the DNA-targeting system is or comprises a zinc-finger DNA binding domain fused to an effector domain. In some embodiments, zinc fingers are custom-designed (i.e. designed by the user), or obtained from a commercial source. Various methods for designing zinc finger proteins are available. For example, methods for designing zinc finger proteins to bind to a target DNA sequence of interest are described, for example in Liu, Q. et al., PNAS, 94(11):5525-30 (1997); Wright, D. A. et al., Nat. Protoc., 1(3):1637-52 (2006); Gersbach, C. A. et al., Acc. Chem. Res., 47(8):2309-18 (2014); Bhakta M. S. et al., Methods Mol. Biol., 649:3-30 (2010); and Gaj et al., Trends Biotechnol, 31(7):397-405 (2013). In addition, various web-based tools for designing zinc finger proteins to bind to a DNA target sequence of interest are publicly available. See, for example, the Zinc Finger Tools design web site from Scripps available on the world wide web at scripps.edu/barbas/zfdesign/zfdesignhome.php. Various commercial services for designing zinc finger proteins to bind to a DNA target sequence of interest are also available. See, for example, the commercially available services or kits offered by Creative Biolabs (world wide web at creative-biolabs.com/Design-and-Synthesis-of-Artificial-Zinc-Finger-Proteins.html), the Zinc Finger Consortium Modular Assembly Kit available from Addgene (world wide web at addgene.org/kits/zfc-modular-assembly/), or the CompoZr Custom ZFN Service from Sigma Aldrich (world wide web at sigmaaldrich.com/life-science/zinc-finger-nuclease-technology/custom-zfn.html). For example, platforms for zinc-finger construction are available that provide specifically targeted zinc fingers for thousands of targets. See, e.g., Gaj et al., Trends in Biotechnology, 2013, 31(7), 397-405. Some gene-specific engineered zinc fingers are available commercially. In some cases, commercially available zinc fingers are used or are custom designed.

In some aspects, the DNA-targeting domain is a domain from Transcription activator-like effectors (TALEs). TALEs are proteins found in Xanthomonas bacteria. TALEs comprise a plurality of repeated amino acid sequences, each repeat having binding specificity for one base in a target sequence. Each repeat comprises a pair of variable residues in position 12 and 13 (repeat variable diresidue; RVD) that determine the nucleotide specificity of the repeat. In some embodiments, RVDs associated with recognition of the different nucleotides are HD for recognizing C, NG for recognizing T, NI for recognizing A, NN for recognizing G or A. NS for recognizing A, C, G or T, HG for recognizing T, IG for recognizing T, NK for recognizing G, HA for recognizing C. ND for recognizing C, HI for recognizing C, HN for recognizing G, NA for recognizing G. SN for recognizing G or A and YG for recognizing T, TL for recognizing A, VT for recognizing A or G and SW for recognizing A. In some embodiments, RVDs can be mutated towards other amino acid residues in order to modulate their specificity towards nucleotides A. T. C and G and in particular to enhance this specificity. Binding domains with similar modular base-per-base nucleic acid binding properties can also be derived from different bacterial species. These alternative modular proteins may exhibit more sequence variability than TALE repeats.

In some embodiments, a “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a TALE protein. TALE proteins may be designed to bind to a target site using canonical or non-canonical RVDs within the repeat units. Sec, e.g., U.S. Pat. Nos. 8,586,526 and 9,458,205.

In some embodiments, a TALE is a fusion protein comprising a nucleic acid binding domain derived from a TALE and an effector domain. In some embodiments, one or more sites in the FXN locus can be targeted by engineered TALEs.

Zinc finger and TALE DNA-binding domains can be engineered to bind to a predetermined nucleotide sequence, for example via engineering (altering one or more amino acids) of the recognition helix region of a zinc finger protein, by engineering of the amino acids in a TALE repeat involved in DNA binding (the repeat variable diresidue or RVD region), or by systematic ordering of modular DNA-binding domains, such as TALE repeats or ZFP domains. Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also WO 98/53058; WO 98/53059; WO 98/53060; WO 02/016536 and WO 03/016496.

### E. Effector Domains

In some embodiments, the DNA-targeting system further comprises at least one effector domain. In some embodiments, the DNA-targeting domain or a component thereof is fused to the at least one effector domain. In some embodiments, provided herein is a DNA-targeting system comprising a fusion protein comprising: (a) a DNA-targeting domain capable of being targeted to a target site at a FXN locus or a regulatory element thereof, such as any described herein, and (b) at least one effector domain. In some aspects, the effector domain leads to an increase in transcription of FXN, or is capable of increasing transcription of FXN. In some aspects, the effector domain comprises a transcription activation domain.

In some embodiments, the DNA-targeting domain comprises a Cas-gRNA combination comprising (a) a Cas protein or a variant thereof and (b) at least one gRNA, and the component thereof fused to the at least one effector domain is the Cas protein or a variant thereof.

In some aspects, the effector domain activates, induces, catalyzes, or leads to demethylation and/or increased transcription of FXN when ectopically recruited to FXN or a DNA regulatory element thereof. Exemplary fusion of DNA-targeting domain and at least one effector domain include fusing dCas9 with transcriptional activators such as VP64 (a polypeptide composed of four tandem copies of VP16, a 16 amino acid transactivation domain of the Herpes simplex virus) can result in robust induction of gene expression.

In some aspects, the effector domain activates, induces, catalyzes, or leads to demethylation and/or increased transcription of FXN when ectopically recruited to FXN or a DNA regulatory element thereof. In some embodiments, the effector domain induces, catalyzes or leads to transcription activation, transcription co-activation, transcription elongation, transcription de-repression, histone modification, nucleosome remodeling, chromatin remodeling, reversal of heterochromatin formation, DNA demethylation, or DNA base oxidation. In some embodiments, the effector domain induces, catalyzes or leads to transcription de-repression, DNA demethylation or DNA base oxidation. In some embodiments, the effector domain induces transcription de-repression. In some embodiments, the effector domain induces transcription activation. In some embodiments, the effector domain has one of the aforementioned activities itself (i.e. acts directly). In some embodiments, the effector domain recruits and/or interacts with a polypeptide domain that has one of the aforementioned activities (i.e. acts indirectly).

### 1. Exemplary Effector Domains

In some embodiments, the effector domain induces, catalyzes or leads to transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation. In some embodiments, the effector domain induces, catalyzes or leads to transcription activation, transcription co-activation, or transcription elongation. In some embodiments, the effector domain induces transcription activation. In some embodiments, the effector domain activates transcription from one or more regulatory elements (e.g., promoters and/or enhancers) from the target locus, e.g., FXN. In some embodiments, the effector domain induces transcription activation. In some embodiments, the effector domain has one of the aforementioned activities itself (i.e. acts or catalyzes directly). In some embodiments, the effector domain recruits and/or interacts with another cellular component (e.g., transcription factor) that has one of the aforementioned activities (i.e. acts or catalyzes indirectly).

In some aspects, gene expression of endogenous mammalian genes, such as human genes, can be achieved by targeting a fusion protein comprising a DNA-targeting domain, such as a dCas9, and an effector domain, such as a transcription activation domain, to mammalian genes or regulatory DNA elements thereof (e.g. a promoter or enhancer), e.g. via one or more gRNAs. Any of a variety of effector domains for transcriptional activation (e.g. transcription activation domains) are known and can be used in accord with the provided embodiments. Transcription activation domains, as well as activation of target genes by Cas fusion proteins (with a variety of Cas molecules) and the transcription activation domains, are described, for example, in WO 2014/197748, WO 2016/130600, WO 2017/180915, WO 2021/226555, WO 2021/226077, WO 2013/176772, WO 2014/152432, WO 2014/093661, Adli, M. Nat. Commun. 9, 1911 (2018), Perez-Pinera et al. Nat. Methods 10, 973-976 (2013), Mali et al. Nat. Biotechnol. 31, 833-838 (2013), and Maeder et al. Nat. Methods 10, 977-979 (2013).

In some embodiments, the effector domain comprises a transcriptional activator domain described in WO 2021/226077.

In some aspects, activation or increase in gene expression of FXN is achieved by targeting a fusion protein comprising a DNA-targeting domain, such as a dCas9, and an effector domain, such as a transcription activation domain, to a FXN locus or regulatory DNA elements thereof (e.g. a promoter or enhancer) via one or more gRNAs. In some aspects, the one or more target sites of the one or more gRNA is at a FXN locus or regulatory DNA elements thereof (e.g., a promoter or enhancer), for example, as described herein, for example, in Section II.A and II.B. Any of a variety of effector domains for transcriptional activation (e.g. transcription activation domains) are known and can be used in accord with the provided embodiments.

The effector domain may have transcription activation activity, i.e., a transactivation domain. For example, gene expression of endogenous mammalian genes, such as human genes, may be achieved by targeting a fusion protein of iCas9 and a transactivation domain to mammalian promoters via combinations of gRNAs. The transactivation domain may include a VP16 protein, multiple VP16 proteins, such as a VP48 domain or VP64 domain, or p65 domain of NF kappa B transcription activator activity. For example, the fusion protein may be iCas9-VP64. In some embodiments, the effector domain may comprise a VP64 domain. For example, dCas9-VP64 can be targeted to a target site by one or more gRNAs to activate a gene. VP64 is a polypeptide composed of four tandem copies of VP16, a 16 amino acid transactivation domain of the Herpes simplex virus. VP64 domains, including in dCas fusion proteins, have been described, for example, in WO 2014/197748, WO 2013/176772, WO 2014/152432, and WO 2014/093661.

In some embodiments, the effector domain comprises at least one VP16 domain, or a VP16 tetramer (“VP64”) or a variant thereof. In some embodiments, the effector domain comprises at least one VP16 domain, or a VP16 tetramer (“VP64”) or a variant thereof. In some embodiments, the effector domain comprises the sequence set forth in SEQ ID NO:81 or 83, a domain thereof, a portion thereof, or a variant thereof, or an amino acid sequence that has at least 90%, 91%, 92%, 93%, 94%, 95%, 96%, 97%, 98%, or 99% sequence identity to any of the foregoing. An exemplary VP64 domain is set forth in SEQ ID NO:81. In some embodiments, the effector domain comprises the sequence set forth in SEQ ID NO:81, a domain thereof, a portion thereof, or a variant thereof, or an amino acid sequence that has at least 90%, 91%, 92%, 93%, 94%, 95%, 96%, 97%, 98%, or 99% sequence identity to any of the foregoing. An exemplary VP64 domain is set forth in SEQ ID NO:83. In some embodiments, the effector domain comprises the sequence set forth in SEQ ID NO:83, a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain may comprise a p65 activation domain (p65AD). p65AD is the principal transactivation domain of the 65 kDa polypeptide of the nuclear form of the NF-KB transcription factor. An exemplary sequence of human transcription factor p65 is available at the Uniprot database under accession number Q04206. p65 domains, including in dCas fusion proteins, have been described, for example in WO 2017/180915 and Chavez, A. et al. Nat. Methods 12, 326-328 (2015). An exemplary p65 activation domain is set forth in SEQ ID NO:134. In some embodiments, the effector domain comprises the sequence set forth in SEQ ID NO:134, a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain may comprise a R transactivator (Rta) domain. Rta is an immediate-early protein of Epstein-Barr virus (EBV), and is a transcriptional activator that induces lytic gene expression and triggers virus reactivation. The Rta domain, including in dCas fusion proteins, has been described, for example in WO 2017/180915 and Chavez, A. et al. Nat. Methods 12, 326-328 (2015). An exemplary Rta domain is set forth in SEQ ID NO:135. In some embodiments, the effector domain comprises the sequence set forth in SEQ ID NO:135, a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain may have histone acetyltransferase activity. For example, the effector domain may comprise a domain from p300 or CREB-binding protein (CBP) protein. The effector domain may comprise a p300 domain. p300 functions as a histone acetyltransferase that regulates transcription via chromatin remodeling and is involved with the processes of cell proliferation and differentiation.

“p300 protein,” “EP300,” or “E1A binding protein p300” as used interchangeably herein refers to the adenovirus E1A-associated cellular p300 transcriptional co-activator protein encoded by the EP300 gene. p300 is a highly conserved acetyltransferase involved in a wide range of cellular processes. p300 functions as a histone acetyltransferase that regulates transcription via chromatin remodeling and is involved with the processes of cell proliferation and differentiation.

The p300 domain, including in dCas fusion proteins for gene activation, has been described, for example, in WO 2016/130600 and WO 2017/180915. An exemplary p300 domain sequence is set forth in SEQ ID NO:112 or 125. In some embodiments, the effector domain comprises the sequence set forth in SEQ ID NO:112 or 125, a domain thereof, a portion thereof, or a variant thereof, or an amino acid sequence that has at least 90%, 91%, 92%, 93%, 94%, 95%, 96%, 97%, 98%, or 99% sequence identity to any of the foregoing. In some embodiments, the effector domain comprises p300 or a domain thereof, a portion thereof, or a variant thereof. In some embodiments, the effector domain comprises the sequence set forth in SEQ ID NO:112 or 125, or a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain may comprise a HSF1 domain. HSF1 is a gene that encodes Heat shock factor protein 1. HSF1, including in dCas fusion proteins for gene activation, has been described, for example, in WO 2021/226555, WO 2015/089427, and Konermann et al. Nature 517(7536):583-8 (2015). An exemplary HSF1 domain is set forth in SEQ ID NO:136. In some embodiments, the effector domain comprises the sequence set forth in SEQ ID NO:136, a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain may comprise a eukaryotic release factor domain, for example from eukaryotic release factor 1 (ERF1) or eukaryotic release factor 3 (ERF3). The effector domain may have transcription release factor activity. The effector domain may have eukaryotic release factor 1 (ERF1) activity or eukaryotic release factor 3 (ERF3) activity.

In some embodiments, the effector domain may comprise the tripartite activator VP64-p65-Rta (also known as VPR). VPR comprises three transcription activation domains (VP64, p65, and Rta) fused by short amino acid linkers, and can effectively upregulate target gene expression. VPR, including in dCas fusion proteins for gene activation, has been described, for example, in WO 2021/226555 and Chavez, A. et al. Nat. Methods 12, 326-328 (2015). An exemplary VPR polypeptide is set forth in SEQ ID NO:137. In some embodiments, the effector domain comprises the sequence set forth in SEQ ID NO: 137, a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain may comprise VPH. VPH is a polypeptide comprising VP64, mouse p65, and HSF1. VPH, including in dCas fusion proteins for gene activation, has been described, for example, in WO 2021/226555. An exemplary VPH polypeptide is set forth in SEQ ID NO: 138. In some embodiments, the effector domain comprises the sequence set forth in SEQ ID NO: 138, a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain may have demethylase activity. The effector domain can include an enzyme that removes methyl (CH3-) groups from nucleic acids, proteins (in particular histones), and other molecules. Alternatively, the effector can convert the methyl group to hydroxymethylcytosine in a mechanism for demethylating DNA. The effector domain can catalyze this reaction. For example, the effector domain that catalyzes this reaction may comprise a domain from a TET protein, for example TET1 (Ten-eleven translocation methylcytosine dioxygenase 1). TET1, including in dCas fusion proteins for gene activation, has been described, for example, in WO 2021/226555. An exemplary TET1 catalytic domain is set forth in SEQ ID NO:139. In some embodiments, the effector domain comprises the sequence set forth in SEQ ID NO:139, a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain may comprise a LSD1 domain. LSD1 (also known as Lysine-specific histone demethylase 1A) is a histone demethylase that can demethylate lysine residues of histone H3, thereby acting as a coactivator or a corepressor, depending on the context. LSD1, including in dCas fusion proteins, has been described, for example, in WO 2013/176772, WO 2014/152432, and Kearns, N. A. et al. Nat. Methods. 12(5):401-403 (2015). An exemplary LSD1 polypeptide is set forth in SEQ ID NO:140. In some embodiments, the effector domain comprises the sequence set forth in SEQ ID NO: 140, a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain may comprise a SunTag domain. SunTag is a repeating peptide array, which can recruit multiple copies of an antibody-fusion protein that binds the repeating peptide. The antibody-fusion protein may comprise an additional effector domain, such as a transcription activation domain (e.g. VP64), to induce increased transcription of the target gene. SunTag, including in dCas fusion proteins for gene activation, has been described, for example, in WO 2016/011070 and Tanenbaum, M. et al. Cell. 159(3):635-646 (2014). An exemplary SunTag effector domain includes a repeating GCN4 peptide having the amino acid sequence LLPKNYHLENEVARLKKLVGER (SEQ ID NO:152) separated by linkers having the amino acid sequence GGSGG (SEQ ID NO:153). In some embodiments, the effector domain comprises the sequence set forth in SEQ ID NO: 152, a domain thereof, a portion thereof, or a variant thereof, or an amino acid sequence that has at least 90%, 91%, 92%, 93%, 94%, 95%, 96%, 97%, 98%, or 99% sequence identity to any of the foregoing. In some embodiments, the SunTag effector domain recruits an antibody-fusion protein that comprises VP64 and binds the GCN4 peptide.

Exemplary fusion of DNA-targeting domain and at least one effector domain include fusing dCas9 with transcriptional activators such as VP64 (a polypeptide composed of four tandem copies of VP16, a 16 amino acid transactivation domain of the Herpes simplex virus) can result in robust induction of gene expression. Exemplary effector domains, such as transcriptional regulators, that can be employed in the provided embodiments include those described in, for example, WO 2014/197748, WO 2016/130600, WO 2017/180915, WO 2021/226555, WO 2021/226077, WO 2013/176772, WO 2014/152432, WO 2014/093661, WO 2021/226077, Adli Nat. Commun. 9, 1911 (2018), Perez-Pinera et al. Nat. Methods 10, 973-976 (2013), Mali et al. Nat. Biotechnol. 31, 833-838 (2013), and Maeder et al. Nat. Methods 10, 977-979 (2013) and Tycko et al., Cell (2020) 183:2020-2035.

In some embodiments, the effector domain is selected from a p65 activation domain, a p300 domain, DPOLA, ENL, FOXO3, HSH2D, NCOA2, NCOA3, PSA1, PYGO1, RBM39, HERC2, DMD, or NOTCH2, or a domain thereof, a portion thereof or a variant thereof. In some embodiments, the effector domain comprises a sequence selected from any one of SEQ ID NOS:113-125, or a domain thereof, a portion thereof, or an amino acid sequence that has at least 90%, 91%, 92%, 93%, 94%, 95%, 96%, 97%, 98%, or 99% sequence identity to any of the foregoing. In some embodiments, the effector domain comprises a sequence selected from any one of SEQ ID NOS: 100-112, or a domain thereof, a portion thereof, or an amino acid sequence that has at least 90%, 91%, 92%, 93%, 94%, 95%, 96%, 97%, 98%, or 99% sequence identity to any of the foregoing. In some embodiments, the variant thereof comprises a truncation thereof.

In some embodiments, the effector domain comprises DPOLA (i.e. DNA polymerase alpha catalytic subunit) or a domain thereof, a portion thereof, or a variant thereof. In some aspects, DPOLA refers to the DNA polymerase alpha catalytic subunit protein encoded by the human POLA1 gene. DPOLA plays an essential role in the initiation of DNA synthesis. An exemplary human DPOLA sequence is set forth in SEQ ID NO: 113. An exemplary DPOLA effector domain sequence is set forth in SEQ ID NO: 100. In some embodiments, the effector domain comprises the sequence set forth in SEQ ID NO:100 or 113, a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain comprises ENL (i.e. protein ENL) or a domain thereof, a portion thereof, or a variant thereof. In some aspects, ENL refers to the protein ENL protein encoded by the human MLLT1 gene. ENL functions as a chromatin reader component of the super elongation complex (SEC), a complex which increases the catalytic rate of RNA polymerase II transcription. An exemplary human ENL sequence is set forth in SEQ ID NO:114. An exemplary ENL effector domain sequence is set forth in SEQ ID NO: 101. In some embodiments, the effector domain comprises the sequence set forth in SEQ ID NO:101 or 114, or a domain thereof, a portion thereof, or a variant thereof or an amino acid sequence that has at least 90%, 91%, 92%, 93%, 94%, 95%, 96%, 97%, 98%, or 99% sequence identity to any of the foregoing.

In some embodiments, the effector domain comprises FOXO3 (i.e. forkhead box protein 03) or a domain thereof, a portion thereof, or a variant thereof. In some aspects, FOXO3 refers to the protein encoded by the human FOXO3 gene. Forkhead box protein 03 functions as a transcriptional activator that recognizes and binds to specific DNA sequences. An exemplary human FOXO3 sequence is set forth in SEQ ID NO:115. An exemplary FOXO3 effector domain sequence is set forth in SEQ ID NO:102. In some embodiments, the effector domain comprises the sequence set forth in SEQ ID NO: 102 or 115, or a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain comprises HSH2D (i.e. hematopoietic SH2 domain-containing protein), or a domain thereof, a portion thereof, or a variant thereof. In some aspects, HSH2D refers to the protein encoded by the human HSH2D gene. HSH2D functions as an adapter protein involved in tyrosine kinase signaling. An exemplary human HSH2D sequence is set forth in SEQ ID NO:116. An exemplary HSH2D effector domain sequence is set forth in SEQ ID NO:103. In some embodiments, the effector domain comprises the sequence set forth in SEQ ID NO:103 or 116, or a domain thereof, a portion thereof, or a variant thereof or an amino acid sequence that has at least 90%, 91%, 92%, 93%, 94%, 95%, 96%, 97%, 98%, or 99% sequence identity to any of the foregoing.

In some embodiments, the effector domain comprises NCOA2 (i.e. nuclear receptor coactivator 2), or a domain thereof, a portion thereof, or a variant thereof. In some aspects, NCOA2 refers to the protein encoded by the human NCOA2 gene. NCOA2 functions as a transcriptional coactivator for steroid receptors and nuclear receptors. An exemplary human NCOA2 sequence is set forth in SEQ ID NO:117. An exemplary NCOA2 effector domain sequence is set forth in SEQ ID NO:104. In some embodiments, the effector domain comprises the sequence set forth in SEQ ID NO:104 or 117, or a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain comprises NCOA3 (i.e. nuclear receptor coactivator 3), or a domain thereof, a portion thereof, or a variant thereof. In some aspects, NCOA3 refers to the protein encoded by the human NCOA3 gene. NCOA3 functions as a transcriptional coactivator for steroid receptors and nuclear receptors. An exemplary human NCOA3 sequence is set forth in SEQ ID NO:118. An exemplary NCOA3 effector domain sequence is set forth in SEQ ID NO:105. In some embodiments, the effector domain comprises the sequence set forth in SEQ ID NO: 105 or 118, or a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain comprises PSA1 (i.e. proteasome subunit alpha type-1), or a domain thereof, a portion thereof, or a variant thereof. In some aspects, PSA1 refers to the protein encoded by the human PSMA1 gene. PSA1 functions as a component of the 20S core proteasome complex, which facilitates proteolytic degradation of intracellular proteins. An exemplary human PSA1 sequence is set forth in SEQ ID NO:119. An exemplary PSA1 effector domain sequence is set forth in SEQ ID NO: 104. In some embodiments, the effector domain comprises the sequence set forth in SEQ ID NO:106 or 119, or a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain comprises PYGO1 (i.e. pygopus homolog 1), or a domain thereof, a portion thereof, or a variant thereof. In some aspects, PYGO1 refers to the protein encoded by the human PYGO1 gene. PYGO1 is involved in Wnt pathway signal transduction. An exemplary human PYGO1 sequence is set forth in SEQ ID NO:120. An exemplary PYGO1 effector domain sequence is set forth in SEQ ID NO: 107. In some embodiments, the effector domain comprises the sequence set forth in SEQ ID NO:107 or 120, or a domain thereof, a portion thereof, or a variant thereof or an amino acid sequence that has at least 90%, 91%, 92%, 93%, 94%, 95%, 96%, 97%, 98%, or 99% sequence identity to any of the foregoing.

In some embodiments, the effector domain comprises RBM39 (i.e. RNA-binding protein 39), or a domain thereof, a portion thereof, or a variant thereof. In some aspects, RBM39 refers to the protein encoded by the human RBM39 gene. RBM39 functions as a RNA-binding protein that acts as a pre-mRNA splicing factor. An exemplary human RBM39 sequence is set forth in SEQ ID NO: 121. An exemplary RBM39 effector domain sequence is set forth in SEQ ID NO:108. In some embodiments, the effector domain comprises the sequence set forth in SEQ ID NO:108 or 121, or a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain comprises HERC2 (i.e. E3 ubiquitin-protein ligase HERC2), or a domain thereof, a portion thereof, or a variant thereof. In some aspects, HERC2 refers to the protein encoded by the human HERC2 gene. HERC2 functions as a regulator of ubiquitin-dependent retention of repair proteins on damaged chromosomes. An exemplary human HERC2 sequence is set forth in SEQ ID NO: 122. An exemplary HERC2 effector domain sequence is set forth in SEQ ID NO: 109. In some embodiments, the effector domain comprises the sequence set forth in SEQ ID NO: 109 or 122, or a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

In some embodiments, the effector domain comprises NOTCH2 (i.e. neurogenic locus notch homolog protein 2), or a domain thereof, a portion thereof, or a variant thereof. In some aspects, NOTCH2 refers to the protein encoded by the human NOTCH2 gene. NOTCH2 functions as a receptor for membrane-bound ligands such as Delta-1 to regulate cell-fate determination. An exemplary human NOTCH2 sequence is set forth in SEQ ID NO:124. An exemplary NOTCH2 effector domain sequence is set forth in SEQ ID NO: 111. In some embodiments, the effector domain comprises the sequence set forth in SEQ ID NO:111 or 124, or a domain thereof, a portion thereof, or a variant thereof, or an amino acid sequence that has at least 90%, 91%, 92%, 93%, 94%, 95%, 96%, 97%, 98%, or 99% sequence identity to any of the foregoing.

The effector domain may have transcription repression activity. The effector domain may have a Kruppel associated box activity, such as a KRAB domain, ERF repressor domain activity, Mxil repressor domain activity, SID4X repressor domain activity, Mad-SID repressor domain activity or TATA box binding protein activity. For example, the fusion protein may be dCas9-KRAB.

The effector domain may have transcription release factor activity. The effector domain may have eukaryotic release factor 1 (ERF1) activity or eukaryotic release factor 3 (ERF3) activity.

The effector domain may have histone modification activity. The effector domain may have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300.

The effector domain may have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases are deoxyribonuclease and ribonuclease.

The effector domain may have nucleic acid association activity or nucleic acid binding protein-DNA-binding domain (DBD) is an independently folded protein domain that contains at least one motif that recognizes double- or single-stranded DNA. A DBD can recognize a specific DNA sequence (a recognition sequence) or have a general affinity to DNA. nucleic acid association region selected from the group consisting of helix-turn-helix region, leucine zipper region, winged helix region, winged helix-turn-helix region, helix-loop-helix region, immunoglobulin fold, B3 domain, Zinc finger, HMG-box, Wor3 domain, TAL effector DNA-binding domain.

The effector domain may have methylase activity, which involves transferring a methyl group to DNA, RNA, protein, small molecule, cytosine or adenine. The effector domain may include a DNA methyltransferase.

The effector domain may have demethylase activity. The effector domain may include an enzyme that remove methyl (CH3-) groups from nucleic acids, proteins (in particular histones), and other molecules. Alternatively, the effector domain may covert the methyl group to hydroxymethylcytosine in a mechanism for demethylating DNA.

### F. Fusion Proteins

Provided are fusion proteins that include (1) a DNA-targeting domain or a component thereof and (2) at least one effector domain. In some aspects, the DNA-targeting domain or component thereof (e.g., a protein or polypeptide component of the DNA-targeting domain, such as the Cas component of the Cas-gRNA combination) can be any described herein, for example, in Section II.C. In some aspects, the at least one effector domain can be any described herein, for example, in Section II.D. In some aspects, the fusion protein is targeted at a target site, for example, one or more target sites at a FXN locus, such as described in Section II.A, by the DNA-targeting domain.

Provided are fusion proteins that include (1) a DNA-targeting domain or a component thereof and (2) at least one effector domain, wherein: the DNA-targeting domain or a component thereof binds to a target site in a regulatory DNA element of a frataxin (FXN) locus; and the effector domain induces, catalyzes or leads to transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation. In some embodiments, the fusion protein comprises any of the effector domains described herein.

In some embodiments, binding of the DNA-targeting domain or a component thereof to the target site does not introduce a genetic disruption or a DNA break at or near the target site. In some embodiments, the DNA-targeting domain comprises a Clustered Regularly Interspaced Short Palindromic Repeats associated (Cas)-guide RNA (gRNA) combination comprising (a) a Cas protein or a variant thereof and (b) at least one gRNA; a zinc finger protein (ZFP); a transcription activator-like effector (TALE); a meganuclease; a homing endonuclease; or a I-SceI enzymes or a variant thereof, such as a catalytically inactive variant thereof. In some embodiments, the DNA-targeting domain comprises a Cas-gRNA combination comprising a Cas protein or a variant thereof and at least one gRNA, and the component of the DNA-targeting domain is a Cas protein or a variant thereof. In some embodiments, the variant Cas protein lacks nuclease activity or is a deactivated Cas (dCas) protein.

In some embodiments, the gRNA is capable of complexing with the Cas protein or variant thereof. In some embodiments, the Cas protein or a variant thereof is a Cas9 protein or a variant thereof. In some embodiments, the variant Cas protein is a variant Cas9 protein that lacks nuclease activity or that is a deactivated Cas9 (dCas9) or a nuclease-inactive Cas9 (iCas9) protein. In some aspects, the dCas9 or iCas9 component of the fusion protein includes any described herein, for example, in Section II.C.1.

In some embodiments, the Cas9 protein or a variant thereof is a Staphylococcus aureus Cas9 (SaCas9) protein or a variant thereof. In some embodiments, the variant Cas9 is a Staphylococcus aureus dCas9 protein (dSaCas9) that comprises at least one amino acid mutation selected from D10A and N580A, with reference to numbering of positions of SEQ ID NO:73. In some embodiments, the variant Cas9 protein comprises the sequence set forth in SEQ ID NO:72, or an amino acid sequence that has at least 90%, 91%, 92%, 93%, 94%, 95%, 96%, 97%, 98%, or 99% sequence identity thereto.

In some embodiments, the Cas9 protein or variant thereof is a Streptococcus pyogenes Cas9 (SpCas9) protein or a variant thereof. In some embodiments, the variant Cas9 is a Streptococcus pyogenes dCas9 (dSpCas9) protein that comprises at least one amino acid mutation selected from D10A and H840A, with reference to numbering of positions of SEQ ID NO:79. In some embodiments, the variant Cas9 protein comprises the sequence set forth in SEQ ID NO:78, or an amino acid sequence that has at least 90%, 91%, 92%, 93%, 94%, 95%, 96%, 97%, 98%, or 99% sequence identity thereto.

In some embodiments, the DNA-targeting domain of the fusion protein is a zinc finger protein (ZFP); a transcription activator-like effector (TALE); a meganuclease; a homing endonuclease; or a I-SceI enzymes or a variant thereof, such as a catalytically inactive variant thereof. In some aspects, the DNA-targeting domain of the fusion protein is targeted to one or more target sites at a FXN locus, such as one or more target sites described herein, for example, in Section II.A. In some aspects, the DNA-targeting domain of the fusion protein is a zinc finger protein (ZFP); a transcription activator-like effector (TALE); a meganuclease; a homing endonuclease; or a I-SceI enzymes or a variant thereof that is capable of binding to a target site at a FXN locus described herein, in a sequence-specific manner.

In some embodiments, the regulatory DNA element is an enhancer. In some embodiments, the target site is located within the genomic coordinates human genome assembly GRCh38 (hg38) chr9:69,027,282-69,028,497. In some embodiments, the target site is located within the genomic coordinates hg38 chr9:69,027,615-69,028,101. In some embodiments, the target site comprises the sequence set forth in SEQ ID NO:21, a contiguous portion thereof of at least 14 nt, or a complementary sequence of any of the foregoing.

In some embodiments, the regulatory DNA element is a promoter. In some embodiments, the target site is located within the genomic coordinates hg38 chr9:68,940,179-69,205,519. In some embodiments, the target site comprises a sequence selected from any one of SEQ ID NOS: 1-10, a contiguous portion thereof of at least 14 nt, or a complementary sequence of any of the foregoing. In some embodiments, the target site comprises a sequence selected from any one of SEQ ID NOS: 11-20, a contiguous portion thereof of at least 14 nt, or a complementary sequence of any of the foregoing.

In some embodiments, the effector domain induces, catalyzes or leads to transcription activation, transcription co-activation, transcription elongation. In some embodiments, the effector domain induces transcription activation. In some embodiments, the effector domain comprises at least one VP16 domain, or a VP16 tetramer (“VP64”) or a variant thereof. In some embodiments, the effector domain comprises the sequence set forth in SEQ ID NO:81 or 83, or a portion thereof, or an amino acid sequence that has at least 90%, 91%, 92%, 93%, 94%, 95%, 96%, 97%, 98%, or 99% sequence identity to any of the foregoing.

In some embodiments, the effector domain is selected from a p65 activation domain, a p300 domain, DPOLA, ENL, FOXO3, HSH2D, NCOA2, NCOA3, PSA1, PYGO1, RBM39, HERC2, DMD, or NOTCH2, or a domain thereof, a portion thereof or a variant thereof. In some embodiments, the effector domain comprises a sequence selected from any one of SEQ ID NOS:113-125, or a domain thereof, a portion thereof, or an amino acid sequence that has at least 90%, 91%, 92%, 93%, 94%, 95%, 96%, 97%, 98%, or 99% sequence identity to any of the foregoing. In some embodiments, the effector domain comprises a sequence selected from any one of SEQ ID NOS: 100-112, or a domain thereof, a portion thereof, or an amino acid sequence that has at least 90%, 91%, 92%, 93%, 94%, 95%, 96%, 97%, 98%, or 99% sequence identity to any of the foregoing. In some embodiments, the variant thereof comprises a truncation thereof. In some aspects, the effector domain comprises any one of the effector domains described herein.

In some embodiments, the at least one effector domain is fused to the N-terminus, the C-terminus, or both the N-terminus and the C-terminus, of the DNA-targeting domain or a component thereof (such as a protein or polypeptide component thereof, for example, a Cas component of a Cas-gRNA combination).

In some embodiments, the at least one effector domain is fused to the N-terminus, the C-terminus, or both the N-terminus and the C-terminus, of the DNA-targeting domain or a component thereof. In some embodiments, the DNA-targeting system also includes one or more linkers connecting the DNA-targeting domain or a component thereof to the at least one effector domain. In some embodiments, the DNA-targeting system further comprises one or more nuclear localization signals (NLS).

In some embodiments, the fusion protein comprises the sequence set forth in SEQ ID NO:85, 159-173, or an amino acid sequence that has at least 90%, 91%, 92%, 93%, 94%, 95%. 96%, 97%, 98%, or 99% sequence identity thereto. In some embodiments, the NLS comprises the sequence set forth in SEQ ID NO:85, 159-173, or a portion thereof. In some embodiments, the NLS comprises the sequence set forth in SEQ ID NO:85 or a portion thereof.

In some embodiments, the fusion protein comprises the sequence set forth in SEQ ID NO:77, or an amino acid sequence that has at least 90%, 91%, 92%, 93%, 94%, 95%, 96%, 97%, 98%, or 99% sequence identity thereto.

In some embodiments, the fusion protein further comprises one or more linkers connecting the DNA-targeting domain or a component thereof to the at least one effector domain, and/or further comprises one or more nuclear localization signals (NLS).

In some embodiments, the fusion protein comprises the sequence set forth in SEQ ID NO:71, or an amino acid sequence that has at least 90%, 91%, 92%, 93%, 94%, 95%, 96%, 97%, 98%, or 99% sequence identity thereto. In some embodiments, the fusion protein comprises the sequence set forth in SEQ ID NO:77, or an amino acid sequence that has at least 90%, 91%, 92%, 93%, 94%, 95%, 96%, 97%, 98%, or 99% sequence identity thereto.

In some embodiments, the fusion protein includes at least one linker. A linker may be included anywhere in the polypeptide sequence of the fusion protein, for example, between the effector domain and the DNA-targeting domain or a component thereof. A linker may be of any length and designed to promote or restrict the mobility of components in the fusion protein.

A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, 30, 35, 40, 45, 50, 55, 60, 65, 70, 75, 80 or 85 amino acids. A linker may comprise an amino acid sequence of less than about 100, 90, 80, 70, 60, 50, or 40 amino acids. A linker may include sequential or tandem repeats of an amino acid sequence that is 2 to 20 amino acids in length. Linkers may be rich in amino acids glycine (G), serine (S), and/or alanine (A). Linkers may include, for example, a GS linker such as (Gly-Gly-Gly-Gly-Ser)n. An exemplary GS linker is represented by the sequence GGGGS (SEQ ID NO:158). A linker may comprise repeats of a sequence, for example as represented by the formula (GGGGS)n, wherein n is an integer that represents the number of times the GGGGS sequence is repeated (e.g. between 1 and 10 times). The number of times a linker sequence is repeated, for example n in a GS linker, can be adjusted to optimize the linker length and achieve appropriate separation of the functional domains. Other examples of linkers may include, for example, Gly-Gly-Gly-Gly-Gly (SEQ ID NO: 154), Gly-Gly-Ala-Gly-Gly (SEQ ID NO:155), Gly/Ser rich linkers such as Gly-Gly-Gly-Gly-Ser-Ser-Ser (SEQ ID NO:156), or Gly/Ala rich linkers such as Gly-Gly-Gly-Gly-Ala-Ala-Ala (SEQ ID NO:157), or Gly-Ser-Gly-Ser-Gly (SEQ ID NO:189).

In some embodiments, the linker is an XTEN linker. In some aspects, an XTEN linker is a recombinant polypeptide (e.g., an unstructured recombinant peptide) lacking hydrophobic amino acid residues. Exemplary XTEN linkers are described in, for example, Schellenberger et al., Nature Biotechnology 27, 1186-1190 (2009) or WO 2021/247570. In some embodiments, an exemplary linker comprises a linker described in WO 2021/247570. In some aspects, the linker is or comprises the sequence set forth in SEQ ID NO:186 or SEQ ID NO:190, or a portion thereof, or an amino acid sequence that has at least 90%, 91%, 92%, 93%, 94%, 95%, 96%, 97%, 98%, or 99% sequence identity to any of the foregoing. In some embodiments, the linker comprises the sequence set forth in SEQ ID NO:186, or a portion thereof, or an amino acid sequence that has at least 90%, 91%, 92%, 93%, 94%, 95%, 96%, 97%, 98%, or 99% sequence identity to any of the foregoing. In some aspects, the linker comprises the sequence set forth in SEQ ID NO: 186, or a contiguous portion of SEQ ID NO:186 of at least 5, 10, 15, 20, 25, 30, 35, 40, 45, 50, 55, 60, 65, 70 or 75 amino acids. In some aspects, the linker consists of the sequence set forth in SEQ ID NO:186, or a contiguous portion of SEQ ID NO:186 of at least 5, 10, 15, 20, 25, 30, 35, 40, 45, 50, 55, 60, 65, 70 or 75 amino acids. In some embodiments, the linker comprises the sequence set forth in SEQ ID NO: 186. In some embodiments, the linker consist of the sequence set forth in SEQ ID NO: 186. In some embodiments, the linker comprises the sequence set forth in SEQ ID NO: 190, or a portion thereof, or an amino acid sequence that has at least 90%, 91%, 92%, 93%, 94%, 95%, 96%, 97%, 98%, or 99% sequence identity to any of the foregoing. In some aspects, the linker comprises the sequence set forth in SEQ ID NO: 190, or a contiguous portion of SEQ ID NO:190 of at least 5, 10, or 15 amino acids. In some aspects, the linker consists of the sequence set forth in SEQ ID NO: 190, or a contiguous portion of SEQ ID NO: 190 of at least 5, 10, or 15 amino acids. In some embodiments, the linker comprises the sequence set forth in SEQ ID NO: 190. In some embodiments, the linker consist of the sequence set forth in SEQ ID NO:190. Appropriate linkers may be selected or designed based rational criteria known in the art, for example as described in Chen et al. Adv. Drug Deliv. Rev. 65(10): 1357-1369 (2013). In some embodiments, a linker comprises the sequence set forth in SEQ ID NO:188, or a portion thereof, or an amino acid sequence that has at least 90%, 91%, 92%, 93%, 94%, 95%, 96%, 97%, 98%, or 99% sequence identity to any of the foregoing.

In some embodiments, a fusion protein described herein comprises one or more nuclear localization sequences (NLSs), such as about or more than about 1, 2, 3, 4, 5, 6, 7, 8, 9, 10, or more NLSs. When more than one NLS is present, each may be selected independently of the others, such that a single NLS may be present in more than one copy and/or in combination with one or more other NLSs present in one or more copies. Non-limiting examples of NLSs include an NLS sequence derived from: the NLS of the SV40 virus large T-antigen, having the sequence PKKKRKV (SEQ ID NO: 159); the NLS from nucleoplasmin (e.g. the nucleoplasmin bipartite NLS) having the sequence KRPAATKKAGQAKKKK (SEQ ID NO:85); the c-myc NLS having the sequence PAAKRVKLD (SEQ ID NO: 160) or RQRRNELKRSP (SEQ ID NO:161); the hRNPA1 M9 NLS having the sequence NQSSNFGPMKGGNFGGRSSGPYGGGGQYFAKPRNQGGY (SEQ ID NO:162); the sequence RMRIZFKNKGKDTAELRRRRVEVSVELRKAKKDEQILKRRNV (SEQ ID NO:163) of the IBB domain from importin-alpha; the sequences VSRKRPRP (SEQ ID NO:164) and PPKKARED (SEQ ID NO: 165) of the myoma T protein; the sequence PQPKKKPL (SEQ ID NO: 166) of human p53; the sequence SALIKKKKKMAP (SEQ ID NO:167) of mouse c-abl IV; the sequences DRLRR (SEQ ID NO: 168) and PKQKKRK (SEQ ID NO: 169) of the influenza virus NS1; the sequence RKLKKKIKKL (SEQ ID NO:170) of the Hepatitis virus delta antigen; the sequence REKKKFLKRR (SEQ ID NO:171) of the mouse Mx1 protein; the sequence KRKGDEVDGVDEVAKKKSKK (SEQ ID NO:172) of the human poly(ADP-ribose) polymerase; and the sequence RKCLQAGMNLEARKTKK (SEQ ID NO:173) of the steroid hormone receptors (human) glucocorticoid. In general, the one or more NLSs are of sufficient strength to drive accumulation of the fusion protein in a detectable amount in the nucleus of a eukaryotic cell. In general, strength of nuclear localization activity may derive from the number of NLSs in the fusion protein, the particular NLS(s) used, or a combination of these factors. Detection of accumulation in the nucleus may be performed by any suitable technique. For example, a detectable marker may be fused to the fusion protein, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of the fusion protein (e.g. an assay for altered gene expression activity in a cell transformed with the DNA-targeting system comprising the fusion protein), as compared to a control condition (e.g. an untransformed cell).

In some embodiments, the NLS comprises the sequence set forth in any one of SEQ ID NO:85 and 160-173, or a portion thereof.

### G. Split Fusion Proteins

In some embodiments, the fusion protein is a split protein, i.e. comprises two or more separate polypeptide domains that interact or self-assemble to form a functional fusion protein. In some aspects, the split fusion protein comprises a dCas9 and an effector domain. In some aspects, the fusion protein comprises a split dCas9-effector domain fusion protein.

In some embodiments, the split fusion protein is assembled from separate polypeptide domains comprising trans-splicing inteins. Inteins are internal protein elements that self-excise from their host protein and catalyze ligation of flanking sequences with a peptide bond. In some embodiments, the split fusion protein is assembled from a first polypeptide comprising an N-terminal intein and a second polypeptide comprising a C-terminal intein. In some embodiments, the N terminal intein is the N terminal Npu Intein set forth in SEQ ID NO:178. In some embodiments, the C terminal intein is the C terminal Npu intein set forth in SEQ ID NO:182.

Also provided are fusion proteins comprising a first polypeptide of a split variant Cas protein comprising an N-terminal fragment of a Cas protein and an N-terminal Intein, and at least one effector domain, wherein the effector domain induces transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation. Also provided are fusion proteins comprising a first polypeptide of a split variant Cas protein comprising an N-terminal fragment of a Cas protein and an N-terminal Intein, and at least one effector domain, wherein the effector domain increases transcription of the FXN locus. In some aspects, the first polypeptide of the split variant Cas protein, and a second polypeptide of the split variant Cas protein comprising a C-terminal fragment of the variant Cas protein and a C-terminal Intein, are present in proximity or present in the same cell, the N-terminal Intein and C-terminal Intein self-excise and ligate the N-terminal fragment and the C-terminal fragment of the variant Cas9 to form a full-length variant Cas9 protein.

Also provided are fusion proteins comprising a second polypeptide of a split variant Cas protein comprising a C-terminal fragment of a Cas protein and a C-terminal Intein and at least one effector domain, wherein the effector domain induces transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation. Also provided are fusion proteins comprising a second polypeptide of a split variant Cas protein comprising a C-terminal fragment of a Cas protein and a C-terminal Intein and at least one effector domain, wherein the effector domain increases transcription of the FXN locus. In some aspects, the second polypeptide of the split variant Cas protein, and a first polypeptide of the split variant Cas protein comprising an N-terminal fragment of the variant Cas protein and an N-terminal Intein, are present in proximity or present in the same cell, the N-terminal Intein and C-terminal Intein self-excise and ligate the N-terminal fragment and the C-terminal fragment of the variant Cas9 to form a full-length variant Cas9 protein.

In some embodiments, the split fusion protein comprises a split dCas9-effector domain fusion protein assembled from two polypeptides. In an exemplary embodiment, the first polypeptide comprises an effector domain catalytic domain and an N-terminal fragment of dSpCas9, followed by an N terminal Npu Intein (effector domain-dSpCas9-573N), and the second polypeptide comprises a C terminal Npu Intein, followed by a C-terminal fragment of dSpCas9 (dSpCas9-573C. The N- and C-terminal fragments of the fusion protein are split at position 573Glu of the dSpCas9 molecule, with reference to SEQ ID NO:79. In some aspects, the N-terminal Npu Intein (SEQ ID NO:178) and C-terminal Npu Intein (set forth in SEQ ID NO:182) may self-excise and ligate the two fragments, thereby forming the full-length dSpCas9-effector domain fusion protein when expressed in a cell.

In some embodiments, the polypeptides of a split protein may interact non-covalently to form a complex that recapitulates the activity of the non-split protein. For example, two domains of a Cas enzyme expressed as separate polypeptides may be recruited by a gRNA to form a ternary complex that recapitulates the activity of the full-length Cas enzyme in complex with the gRNA, for example as described in Wright et al. PNAS 112(10):2984-2989 (2015). In some embodiments, assembly of the split protein is inducible (e.g. light inducible, chemically inducible, small-molecule inducible).

In some aspects, the two polypeptides of a split fusion protein may be delivered and/or expressed from separate vectors, such as any of the vectors described herein. In some embodiments, the two polypeptides of a split fusion protein may be delivered to a cell and/or expressed from two separate AAV vectors, i.e. using a split AAV-based approach, for example as described in WO 2017/197238.

Approaches for the rationale design of split proteins and their delivery, including Cas proteins and fusions thereof, are described, for example, in WO 2016/114972, WO 2017/197238, Zetsche. et al. Nat. Biotechnol. 33(2):139-42 (2015), Wright et al. PNAS 112(10):2984-2989 (2015), Truong. et al. Nucleic Acids Res. 43, 6450-6458 (2015), and Fine et al. Sci. Rep. 5, 10777 (2015).

### H. Exemplary Fusion Proteins

In some aspects, provided are DNA-targeting systems or fusion proteins that comprise a Cas protein or a variant thereof and at least one effector domain, wherein the effector domain increases transcription of the FXN locus.

In some embodiments, the at least one effector domain is fused to the N-terminus, the C-terminus, or both the N-terminus and the C-terminus, of the DNA-targeting domain or a component thereof (such as a protein or polypeptide component thereof, for example, a Cas component of a Cas-gRNA combination). In some embodiments, the DNA-targeting system also includes one or more linkers connecting the DNA-targeting domain or a component thereof to the at least one effector domain, and/or further comprising one or more nuclear localization signals (NLS).

In some aspects, the DNA-targeting system or fusion protein comprises one or more tags, linkers and/or NLS sequences. In some embodiments, exemplary tags, linkers and/or NLS sequences can be any described herein.

In some cases, sequences provided herein, including amino acid sequences for the DNA-targeting systems or fusion proteins provided herein, contain sequences of one or more tags, linkers and/or NLS sequences. In some aspects, it is understood that the exemplary tags, linkers and/or NLS sequences are not required or are not the sole or exclusive tags, linkers and/or NLS sequences that can be employed in the DNA-targeting systems or fusion proteins. In some aspects, sequences containing tags, linkers and/or NLS sequences are exemplary, and are not limited to the specific tags, linkers and/or NLS sequences contained in the described sequences. In some aspects, alternative tags, linkers and/or NLS sequences can be can be employed in the DNA-targeting systems or fusion proteins, or the DNA-targeting system or fusion protein in some cases does not contain or lacks a tag, linker and/or NLS. In some aspects, alternative tags, linkers and/or NLS sequences include other known tags, linkers and/or NLS sequences that have similar function or serve similar purposes.

In some embodiments, the DNA-targeting system or fusion protein comprises the sequence set forth in SEQ ID NO:71, or an amino acid sequence that has at least 90%, 91%, 92%, 93%, 94%, 95%, 96%, 97%, 98%, or 99% sequence identity thereto.

In some embodiments, the DNA-targeting system or fusion protein comprises the sequence set forth in SEQ ID NO:71.

In some embodiments, the DNA-targeting system or fusion protein comprises the sequence set forth in SEQ ID NO:77, or an amino acid sequence that has at least 90%, 91%, 92%, 93%, 94%, 95%, 96%, 97%, 98%, or 99% sequence identity thereto. In some embodiments, the DNA-targeting system or fusion protein comprises the sequence set forth in SEQ ID NO:77.

In some embodiments, the DNA-targeting system or fusion protein comprises, from N- to C-terminal order: a dSaCas9 set forth in SEQ ID NO:72, a linker and/or NLS, and a DPOLA domain set forth in SEQ ID NO:100. In some embodiments, the DNA-targeting system or fusion protein comprises, from N- to C-terminal order: a dSaCas9 set forth in SEQ ID NO:72, a linker and/or NLS, and a ENL domain set forth in SEQ ID NO:101. In some embodiments, the DNA-targeting system or fusion protein comprises, from N- to C-terminal order: a dSaCas9 set forth in SEQ ID NO:72, a linker and/or NLS, and a FOXO3 domain set forth in SEQ ID NO:102. In some embodiments, the DNA-targeting system or fusion protein comprises, from N- to C-terminal order: a dSaCas9 set forth in SEQ ID NO:72, a linker and/or NLS, and a HSH2D domain set forth in SEQ ID NO:103. In some embodiments, the DNA-targeting system or fusion protein comprises, from N- to C-terminal order: a dSaCas9 set forth in SEQ ID NO:72, a linker and/or NLS, and a NCOA2 domain set forth in SEQ ID NO:104. In some embodiments, the DNA-targeting system or fusion protein comprises, from N- to C-terminal order: a dSaCas9 set forth in SEQ ID NO:72, a linker and/or NLS, and a NCOA3 domain set forth in SEQ ID NO:105. In some embodiments, the DNA-targeting system or fusion protein comprises, from N- to C-terminal order: a dSaCas9 set forth in SEQ ID NO:72, a linker and/or NLS, and a PSA1 domain set forth in SEQ ID NO:106. In some embodiments, the DNA-targeting system or fusion protein comprises, from N- to C-terminal order: a dSaCas9 set forth in SEQ ID NO:72, a linker and/or NLS, and a PYGO1 domain set forth in SEQ ID NO:107. In some embodiments, the DNA-targeting system or fusion protein comprises, from N- to C-terminal order: a dSaCas9 set forth in SEQ ID NO:72, a linker and/or NLS, and a RBM39 domain set forth in SEQ ID NO:108. In some embodiments, the DNA-targeting system or fusion protein comprises, from N- to C-terminal order: a dSaCas9 set forth in SEQ ID NO:72, a linker and/or NLS, and a HERC2 domain set forth in SEQ ID NO:109. In some embodiments, the DNA-targeting system or fusion protein comprises, from N- to C-terminal order: a dSaCas9 set forth in SEQ ID NO:72, a linker and/or NLS, and a DMD domain set forth in SEQ ID NO:110. In some embodiments, the DNA-targeting system or fusion protein comprises, from N- to C-terminal order: a dSaCas9 set forth in SEQ ID NO:72, a linker and/or NLS, and a NOTCH2 domain set forth in SEQ ID NO:111. In some embodiments, the DNA-targeting system or fusion protein comprises, from N- to C-terminal order: a dSaCas9 set forth in SEQ ID NO:72, a linker and/or NLS, and a p300 core domain set forth in SEQ ID NO:112.

In some embodiments, the DNA-targeting system or fusion protein comprises, from N- to C-terminal order: a DPOLA domain set forth in SEQ ID NO: 100, a linker and/or NLS, and a dSaCas9 set forth in SEQ ID NO:72. In some embodiments, the DNA-targeting system or fusion protein comprises, from N- to C-terminal order: a dSaCas9 set forth in SEQ ID NO:72, a linker and/or NLS, and a ENL domain set forth in SEQ ID NO:101, a linker and/or NLS, and a dSaCas9 set forth in SEQ ID NO:72. In some embodiments, the DNA-targeting system or fusion protein comprises, from N- to C-terminal order: a dSaCas9 set forth in SEQ ID NO:72, a linker and/or NLS, and a FOXO3 domain set forth in SEQ ID NO: 102, a linker and/or NLS, and a dSaCas9 set forth in SEQ ID NO:72. In some embodiments, the DNA-targeting system or fusion protein comprises, from N- to C-terminal order: a dSaCas9 set forth in SEQ ID NO:72, a linker and/or NLS, and a HSH2D domain set forth in SEQ ID NO: 103, a linker and/or NLS, and a dSaCas9 set forth in SEQ ID NO:72. In some embodiments, the DNA-targeting system or fusion protein comprises, from N- to C-terminal order: a dSaCas9 set forth in SEQ ID NO:72, a linker and/or NLS, and a NCOA2 domain set forth in SEQ ID NO: 104, a linker and/or NLS, and a dSaCas9 set forth in SEQ ID NO:72. In some embodiments, the DNA-targeting system or fusion protein comprises, from N- to C-terminal order: a dSaCas9 set forth in SEQ ID NO:72, a linker and/or NLS, and a NCOA3 domain set forth in SEQ ID NO: 105, a linker and/or NLS, and a dSaCas9 set forth in SEQ ID NO:72. In some embodiments, the DNA-targeting system or fusion protein comprises, from N- to C-terminal order: a dSaCas9 set forth in SEQ ID NO:72, a linker and/or NLS, and a PSA1 domain set forth in SEQ ID NO:106, a linker and/or NLS, and a dSaCas9 set forth in SEQ ID NO:72. In some embodiments, the DNA-targeting system or fusion protein comprises, from N- to C-terminal order: a dSaCas9 set forth in SEQ ID NO:72, a linker and/or NLS, and a PYGO1 domain set forth in SEQ ID NO: 107, a linker and/or NLS, and a dSaCas9 set forth in SEQ ID NO:72. In some embodiments, the DNA-targeting system or fusion protein comprises, from N- to C-terminal order: a dSaCas9 set forth in SEQ ID NO:72, a linker and/or NLS, and a RBM39 domain set forth in SEQ ID NO: 108, a linker and/or NLS, and a dSaCas9 set forth in SEQ ID NO:72. In some embodiments, the DNA-targeting system or fusion protein comprises, from N- to C-terminal order: a dSaCas9 set forth in SEQ ID NO:72, a linker and/or NLS, and a HERC2 domain set forth in SEQ ID NO:109, a linker and/or NLS, and a dSaCas9 set forth in SEQ ID NO:72. In some embodiments, the DNA-targeting system or fusion protein comprises, from N- to C-terminal order: a dSaCas9 set forth in SEQ ID NO:72, a linker and/or NLS, and a DMD domain set forth in SEQ ID NO:110, a linker and/or NLS, and a dSaCas9 set forth in SEQ ID NO:72. In some embodiments, the DNA-targeting system or fusion protein comprises, from N- to C-terminal order: a dSaCas9 set forth in SEQ ID NO:72, a linker and/or NLS, and a NOTCH2 domain set forth in SEQ ID NO: 111, a linker and/or NLS, and a dSaCas9 set forth in SEQ ID NO:72. In some embodiments, the DNA-targeting system or fusion protein comprises, from N- to C-terminal order: a dSaCas9 set forth in SEQ ID NO:72, a linker and/or NLS, and a p300 core domain set forth in SEQ ID NO:112, a linker and/or NLS, and a dSaCas9 set forth in SEQ ID NO:72.

In some aspects, exemplary linkers or NLS sequences can be any described herein.

### I. Combinations of DNA-Targeting Systems or Fusion Proteins

Also provided are combinations, such as combinations of two or more DNA-targeting systems or components thereof. In some aspects, provided herein are combinations of two or more DNA-targeting systems that independently target different target sites at a frataxin (FXN) locus. In some aspects, the two or more DNA-targeting systems each comprise any of the DNA-targeting systems described herein.

In some embodiments, the DNA-targeting domain is a first DNA-targeting domain, and the DNA-targeting system further comprises one or more second DNA-targeting domains. In some embodiments, the first DNA-targeting domain binds a first target site in an enhancer of a FXN locus, and the second DNA-targeting domain binds a second target site in a promoter of a FXN locus.

In some aspects, the provided combination of DNA-targeting systems include two or more DNA-targeting systems, each of which target particular regions of a frataxin (FXN) locus.

Also provided herein is a combination, comprising a first DNA-targeting system comprising any of the DNA-targeting systems described herein, and one or more second DNA-targeting systems that binds to a second target site in a regulatory DNA element of a frataxin (FXN) locus. In some embodiments, the second DNA-targeting system comprises any of the DNA-targeting systems described herein.

Also provided are combinations, such as combinations of two or more DNA-targeting domains or fusion proteins or components thereof. In some aspects, provided herein are combinations of two or more DNA-targeting domains or fusion proteins that independently target different target sites at a frataxin (FXN) locus. In some aspects, the two or more DNA-targeting domains or fusion proteins each comprise any of the DNA-targeting domains or fusion proteins described herein.

In some embodiments, the DNA-targeting domain is a first DNA-targeting domain, and the DNA-targeting domain or fusion protein further comprises one or more second DNA-targeting domains. In some embodiments, the first DNA-targeting domain binds a first target site in an enhancer of a FXN locus, and the second DNA-targeting domain binds a second target site in a promoter of a FXN locus.

In some aspects, the provided combination of DNA-targeting domains or fusion proteins include two or more DNA-targeting domains or fusion proteins, each of which target particular regions of a frataxin (FXN) locus.

Also provided herein is a combination, comprising a first DNA-targeting domain or fusion protein comprising any of the DNA-targeting domains or fusion proteins described herein, and one or more second DNA-targeting domains or fusion proteins that binds to a second target site in a regulatory DNA element of a frataxin (FXN) locus. In some embodiments, the second DNA-targeting domain or fusion protein comprises any of the DNA-targeting domains or fusion proteins described herein.

Also provided herein are DNA-targeting systems that binds to one or more target sites in a regulatory DNA element of a frataxin (FXN) locus, the DNA-targeting system comprising: a first DNA-targeting domain that binds a first target site in an enhancer of a FXN locus, and a second DNA-targeting domain that binds a second target site in a promoter of a FXN locus.

In some aspects, exemplary combination of DNA-targeting systems include: (a) a fusion protein comprising a Cas protein or a variant thereof and (b) a combination of gRNAs, such as a first gRNA that is capable of hybridizing to the target site or is complementary to the first target site and a second gRNA that is capable of hybridizing to the target site or is complementary to the second target site. In some aspects, also provided herein are combinations of DNA-targeting systems comprising one type of Cas protein or variant thereof, such as a dCas9 protein or variant thereof, and two or more different gRNAs, such as a combination of gRNAs, such as any combination of gRNAs described herein. In some aspects, also provided herein are combinations of DNA-targeting systems comprising one type of Cas protein or variant thereof, such as a dCas9 protein or variant thereof, two or more different types of effector domains, and two or more different gRNAs, such as a combination of gRNAs, such as any combination of gRNAs described herein. In some aspects, also provided herein are combinations of DNA-targeting systems comprising two or more different type of Cas protein or variant thereof, such as a dCas9 protein or variant thereof, and two or more different gRNAs, such as a combination of gRNAs, such as any combination of gRNAs described herein. In some aspects, also provided herein are combinations of DNA-targeting systems comprising two or more different types of DNA-targeting domains and one type of effector domain. In some aspects, also provided herein are combinations of DNA-targeting systems comprising two or more different types of DNA-targeting domains and two or more different types of effector domain.

In some embodiments, the first target site is located within the genomic coordinates human genome assembly GRCh38 (hg38) chr9:69,027,282-69,028,497, and the second target site is located within the genomic coordinates hg38 chr9:68,940, 179-69,205,519. In some embodiments, the first target site is located within the genomic coordinates hg38 chr9:69,027,615-69,028,101, and the second target site is located within the genomic coordinates hg38 chr9:68,940,179-69,205,519.

In some embodiments, the first DNA-targeting domain comprises a first Cas-gRNA combination comprising (a) a first Cas protein or a variant thereof and (b) a first gRNA that is capable of hybridizing to the target site or is complementary to the first target site; and the second DNA-targeting domain comprises a second Cas-gRNA combination comprising (a) a second Cas protein or a variant thereof and (b) a second gRNA that is capable of hybridizing to the target site or is complementary to the second target site. In some embodiments, the first DNA-targeting domain comprises a first Cas-gRNA combination comprising (a) a first Cas protein or a variant thereof and (b) a first gRNA comprising at least one gRNA spacer sequence set forth in SEQ ID NO:42 or a contiguous portion thereof of at least 14 nt. In some embodiments, the second DNA-targeting domain comprises a second Cas-gRNA combination comprising (a) a second Cas protein or a variant thereof and (b) a second gRNA comprising at least one gRNA spacer sequence set forth in SEQ ID NO:22 or 28 or a contiguous portion thereof of at least 14 nt.

In some embodiments, the first Cas-gRNA combination comprises (a) a first Cas protein or a variant thereof and (b) a first gRNA comprising at least one gRNA spacer sequence set forth in SEQ ID NO:42 or a contiguous portion thereof of at least 14 nt; and the second Cas-gRNA combination comprises (a) a second Cas protein or a variant thereof and (b) a second gRNA comprising at least one gRNA spacer sequence set forth in SEQ ID NO:22 or a contiguous portion thereof of at least 14 nt. In some embodiments, the first Cas-gRNA combination comprises (a) a first Cas protein or a variant thereof and (b) a first gRNA comprising at least one gRNA spacer sequence set forth in SEQ ID NO:42 or a contiguous portion thereof of at least 14 nt; and the second Cas-gRNA combination comprises (a) a second Cas protein or a variant thereof and (b) a second gRNA comprising at least one gRNA spacer sequence set forth in SEQ ID NO:28 or a contiguous portion thereof of at least 14 nt.

In some embodiments, the first DNA-targeting domain binds a first target site in a promoter of a FXN locus; and the second DNA-targeting domain binds a second target site in a promoter of a FXN locus.

Also provided herein are DNA-targeting systems that binds to one or more target sites in a regulatory DNA element of a frataxin (FXN) locus, the DNA-targeting system comprising: a first DNA-targeting domain that binds a first target site in a promoter of a FXN locus; and a second DNA-targeting domain that binds a second target site in a promoter of a FXN locus.

In some embodiments, the first target site and the second target site independently are located within the genomic coordinates hg38 chr9:68,940,179-69,205,519. In some embodiments, the first target site and the second target site are different.

In some embodiments, the first DNA-targeting domain comprises a first Cas-gRNA combination comprising (a) a first Cas protein or a variant thereof and (b) a first gRNA that is capable of hybridizing to the target site or is complementary to the first target site; and the second DNA-targeting domain comprises a second Cas-gRNA combination comprising (a) a second Cas protein or a variant thereof and (b) a second gRNA that is capable of hybridizing to the target site or is complementary to the second target site. In some embodiments, the first DNA-targeting domain comprises a first Cas-gRNA combination comprising (a) a first Cas protein or a variant thereof and (b) a first gRNA comprising at least one gRNA spacer sequence set forth in SEQ ID NO:22 or a contiguous portion thereof of at least 14 nt. In some embodiments, the second DNA-targeting domain comprises a second Cas-gRNA combination comprising (a) a second Cas protein or a variant thereof and (b) a second gRNA comprising at least one gRNA spacer sequence set forth in SEQ ID NO:28 or a contiguous portion thereof of at least 14 nt.

In some embodiments, the first Cas-gRNA combination comprises (a) a first Cas protein or a variant thereof and (b) a first gRNA comprising at least one gRNA spacer sequence set forth in SEQ ID NO:22 or a contiguous portion thereof of at least 14 nt; and the second Cas-gRNA combination comprises (a) a second Cas protein or a variant thereof and (b) a second gRNA comprising at least one gRNA spacer sequence set forth in SEQ ID NO:28 or a contiguous portion thereof of at least 14 nt.

In some embodiments, the first Cas protein or a variant thereof and/or the second Cas protein or a variant thereof is a variant Cas9 protein that lacks nuclease activity or that is a deactivated Cas9 (dCas9) protein.

In some embodiments, the first variant Cas protein and/or the second variant Cas protein is a Staphylococcus aureus dCas9 protein (dSaCas9) that comprises at least one amino acid mutation selected from D10A and N580A, with reference to numbering of positions of SEQ ID NO:73; or comprises the sequence set forth in SEQ ID NO:72, or an amino acid sequence that has at least 90%, 91%, 92%, 93%, 94%, 95%, 96%, 97%, 98%, or 99% sequence identity thereto.

In some embodiments, the first variant Cas protein and/or the second variant Cas protein is a Streptococcus pyogenes dCas9 (dSpCas9) protein that comprises at least one amino acid mutation selected from D10A and H840A, with reference to numbering of positions of SEQ ID NO:79; or comprises the sequence set forth in SEQ ID NO:78, or an amino acid sequence that has at least 90%, 91%, 92%, 93%, 94%, 95%, 96%, 97%, 98%, or 99% sequence identity thereto.

In some embodiments, the first Cas protein and the second Cas protein are the same. In some embodiments, the first Cas protein and the second Cas protein are identical. In some embodiments, the first Cas protein and the second Cas protein are different. In some embodiments, the first Cas protein and the second Cas protein are from different species.

In some embodiments, the first Cas protein or a variant thereof and/or the second Cas protein or a variant thereof is fused to at least one effector domain.

In some embodiments, the effector domain induces, catalyzes or leads to transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation. In some embodiments, the effector domain induces transcription activation.

In some embodiments, all of the components of the combination of DNA-targeting systems, DNA-targeting domains or fusion proteins provided herein are encoded in one polynucleotide. In some embodiments, all of the components of the combination of DNA-targeting systems, DNA-targeting domains or fusion proteins provided herein are encoded in multiple individual polynucleotides, such as a first polynucleotide and a second polynucleotide. In some aspects, first DNA-targeting system, DNA-targeting domain or fusion protein and the second DNA-targeting system, DNA-targeting domain or fusion protein are encoded in one polynucleotide, such as a first polynucleotide. In some embodiments, the first DNA-targeting system, domain or fusion protein and the second DNA-targeting system, domain or fusion protein are encoded in one polynucleotide, such as a first polynucleotide. In some embodiments, the first Cas protein and the second Cas protein are encoded in a first polynucleotide. In some embodiments, the first Cas protein and the second Cas protein are encoded by the same nucleotide sequence. In some embodiments, the first gRNA and the second gRNA are encoded in a first polynucleotide. In some embodiments, the first Cas protein and the second Cas protein are encoded by the same nucleotide sequence, and the Cas protein, the first gRNA, and the second gRNA are encoded in a first polynucleotide. In some embodiments, the first DNA-targeting domain is encoded in a first polynucleotide and the second DNA-targeting domain is encoded in a second polynucleotide. In some embodiments, the first Cas protein is encoded in a first polynucleotide and the second Cas protein is encoded in a second polynucleotide. In some embodiments, the first gRNA is encoded in a first polynucleotide and the second gRNA is encoded in a second polynucleotide. In some embodiments, the first Cas protein and the first gRNA are encoded in a first polynucleotide, and the second Cas protein and the second gRNA are encoded in a second polynucleotide.

### III. Polynucleotides, Vectors and Delivery of DNA-Targeting Systems

Provided are polynucleotides encoding any of the DNA-targeting systems described herein, any of the gRNAs described herein, any of the combinations described herein, or any of the fusion proteins described herein, or a portion or a component of any of the foregoing. In some aspects, the polynucleotides can encode any of the components of the DNA-targeting systems, and/or any nucleic acid or proteinaceous molecule necessary to carry out aspects of the methods of the disclosure can comprise a vector (e.g., a recombinant expression vector). In some of any embodiments, provided are polynucleotides encoding any of the fusion proteins described herein. Also provided herein are polynucleotides encoding any of the gRNAs or combinations of gRNAs described herein.

### A. Nucleic Acids

Provided are polynucleotides encoding any of the DNA-targeting systems described herein, including a protein component of the DNA-targeting system (e.g., Cas protein or a variant thereof) and the at least one gRNA, such as one or more RNAs.

In some embodiments, provided are polynucleotides comprising the gRNAs described herein. In some embodiments, the gRNA is transcribed from a genetic construct (i.e. vector or plasmid) in the target cell. In some embodiments, the gRNA is produced by in vitro transcription and delivered to the target cell. In some embodiments, the gRNA comprises one or more modified nucleotides for increased stability. In some embodiments, the gRNA is delivered to the target cell pre-complexed as a RNP with the fusion protein.

In some embodiments, a provided polynucleotide encodes a fusion protein as described herein that includes (a) a DNA-targeting domain capable of being targeted to a target site of a target gene as described; and (b) at least one effector domain capable of reducing transcription of the gene. In some embodiments, the fusion protein includes a fusion protein of a Cas protein or variant thereof and at least one effector domain capable of reducing transcription of a gene. In a particular example, the Cas is a deactivated Cas (dCas), such as dCas9. In some embodiments, the dCas9 is a dSpCas9. Examples of such domains and fusion proteins include any as described in Section I.

In some embodiments, the polynucleotide, such as a polynucleotide encoding any of the components of the DNA targeting system, fusion protein and/or gRNA, is DNA. In some embodiments, the polynucleotide, such as a polynucleotide encoding any of the components of the DNA targeting system, fusion protein and/or gRNA, is RNA. In some embodiments, the polynucleotide is mRNA. In some embodiments, the gRNA is provided as RNA and a polynucleotide encoding the fusion protein is mRNA. In some aspects, the mRNA is 5′ capped and/or 3′ polyadenylated. In some embodiments, a polynucleotide provided herein is DNA. In some aspects, the DNA is present in a vector.

In some embodiments, the polynucleotide encodes the fusion protein and one or more gRNAs or a combination of gRNAs.

In some embodiments, the polynucleotide as provided herein can be codon optimized for efficient translation into protein in the eukaryotic cell or animal of interest. For example, codons can be optimized for expression in humans, mice, rats, hamsters, cows, pigs, cats, dogs, fish, amphibians, plants, yeast, insects, and others.

In some embodiments, the polynucleotide comprises the sequence set forth in SEQ ID NO:68 or 74, or a sequence having at least about 80%, 85%, 90%, 91%, 92%, 93%, 94%, 95%, 96%, 97%, 98%, 99%, or 100% sequence identity thereto. In some embodiments, the polynucleotide comprises the sequence set forth in SEQ ID NO:68. In some embodiments, the polynucleotide comprises the sequence set forth in SEQ ID NO:74.

Also provided are polynucleotides encoding a first DNA-targeting system, a first Cas protein and/or a first gRNA of any of the DNA-targeting systems described herein or any of the combinations described herein.

Provided are polynucleotides encoding a second DNA-targeting system, a second Cas protein and/or a second gRNA of any of the DNA-targeting systems described herein or any of the combinations described herein.

Provided are polynucleotides that include any of the polynucleotides described herein, and one or more additional polynucleotides encoding an additional portion or an additional component of any of the DNA-targeting systems described herein, any of the gRNAs described herein, any of the combinations described herein, or any of the fusion proteins described herein, or a portion or a component of any of the foregoing.

Also provided herein are pluralities of polynucleotides, comprising: (a) a polynucleotide encoding a first DNA-targeting system, a first Cas protein and/or a first gRNA of any of the a DNA-targeting system disclosed herein or any of the combinations of gRNAs disclosed herein, and (b) a polynucleotide encoding a second DNA-targeting system, a second Cas protein and/or a second gRNA of any of the DNA-targeting system disclosed herein or any of the combinations of gRNAs disclosed herein.

Provided are pluralities of polynucleotides, that includes a first polynucleotide comprising any of the polynucleotides described herein; and a second polynucleotide comprising any of the polynucleotides described herein.

In some embodiments, the first DNA-targeting domain and the second DNA-targeting domain are encoded in a first polynucleotide. In some embodiments, the first Cas protein and the second Cas protein are encoded in a first polynucleotide. In some embodiments, the first Cas protein and the second Cas protein are encoded by the same nucleotide sequence. In some embodiments, the first gRNA and the second gRNA are encoded in a first polynucleotide. In some embodiments, the first Cas protein and the second Cas protein are encoded by the same nucleotide sequence, and the Cas protein, the first gRNA, and the second gRNA are encoded in a first polynucleotide.

In some embodiments, the first DNA-targeting domain is encoded in a first polynucleotide and the second DNA-targeting domain is encoded in a second polynucleotide. In some embodiments, the first Cas protein is encoded in a first polynucleotide and the second Cas protein is encoded in a second polynucleotide. In some embodiments, the first gRNA is encoded in a first polynucleotide and the second gRNA is encoded in a second polynucleotide. In some embodiments, the first Cas protein and the first gRNA are encoded in a first polynucleotide, and the second Cas protein and the second gRNA are encoded in a second polynucleotide.

### B. Vectors

Provided are vectors that include any of the polynucleotides described herein, any of the pluralities of polynucleotides described herein, or a first polynucleotide or a second polynucleotide of any of the pluralities of polynucleotides described herein, or a portion or a component of any of the foregoing.

Also provided herein is a vector that comprises or contains any of the provided polynucleotides. In some embodiments, the vector comprises a genetic construct, such as a plasmid or an expression vector. The vector can be a self-inactivating vector that either inactivates the viral sequences or the components of the CRISPR machinery or other elements.

In some embodiments, the expression vector comprising the sequence encoding the fusion protein of a DNA-targeting system provided herein further comprises a nucleic acid sequence encoding at least one gRNA. In some embodiments, the expression vector comprises a nucleic acid sequence or combination of nucleic acid sequences encoding two or more gRNAs, such as two gRNAs. In some embodiments, the expression vector comprises a nucleic acid sequence or combination of nucleic acid sequences encoding three gRNAs. In some cases, the sequence encoding the gRNA is operably linked to at least one transcriptional control sequence or transcriptional regulatory sequence (e.g., cis-regulatory sequence) for expression of the gRNA in the cell. In some aspects, DNA encoding the gRNA can be operably linked to a promoter sequence that is recognized by RNA polymerase III (Pol III). Examples of suitable Pol III promoters include, but are not limited to, mammalian U6, U3, H1, and 7SL RNA promoters, or variants thereof. In some aspects, if the expression vector comprises nucleic acid sequences encoding two or more gRNAs, each gRNA is operably linked to an identical Pol III promoter, or different Pol III promoters.

In some embodiments, provided is a vector containing a polynucleotide that encodes a fusion protein comprising a DNA-targeting domain comprising a dCas and at least one effector domain capable of increasing transcription of a gene, and a polynucleotide or combination of polynucleotides encoding a gRNA, or a plurality of gRNAs, such as two, three, or four or more gRNAs, or such as two, three, or four or more different gRNAs. In some embodiments, the dCas is a dCas9, such as dSaCas9 or dSpCas9. In some embodiments, the polynucleotide encodes a fusion protein that includes a dSaCas9 set forth in SEQ ID NO:72. In some embodiments, the polynucleotide encodes a fusion protein that includes a dSpCas9 set forth in SEQ ID NO:78. In some embodiments, the polynucleotide(s) encodes one or more a gRNAs described herein, for example in or a plurality of gRNAs, each gRNA as described in Section II.B.

In some examples, a polynucleotide and/or a vector described herein can comprise one or more transcription and/or translation control elements. Depending on the host/vector system utilized, any of a number of suitable transcription and translation control elements, including constitutive and inducible promoters, transcription enhancer elements, transcription terminators, etc. can be used in the expression vector.

Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

For expressing small RNAs, including guide RNAs used in connection with the DNA-targeting systems, various promoters such as RNA polymerase III promoters, including for example U6 and H1, can be advantageous. Descriptions of and parameters for enhancing the use of such promoters are known in the art, and additional information and approaches include those described in, e.g., Ma, H. et al., Molecular Therapy—Nucleic Acids 3, e161 (2014) doi: 10.1038/mtna.2014.12.

The expression vector can also contain a ribosome binding site for translation initiation and a transcription terminator. The expression vector can also comprise appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some cases, the promoter can be a spatially restricted and/or temporally restricted promoter (e.g., a tissue specific promoter, a cell type specific promoter (e.g. nervous system specific promoter), etc.).

In some examples, vectors can be capable of directing the expression of nucleic acids to which they are operatively linked. Such vectors are referred to herein as “recombinant expression vectors”, or more simply “expression vectors”, which serve equivalent functions.

Exemplary expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia). Other vectors can be used so long as they are compatible with the host cell.

In some embodiments, the vector is a viral vector, such as an adeno-associated virus (AAV) vector, a retroviral vector, a lentiviral vector, or a gammaretroviral vector. n some embodiments, the viral vector is an adeno-associated virus (AAV) vector. In some embodiments, the AAV vector is selected from among an AAV1, AAV2, AAV3, AAV4, AAV5, AAV6, AAV7, AAV8, or AAV9 vector. In some embodiments, the vector is a lentiviral vector. In some embodiments, the vector is a non-viral vector, for example a lipid nanoparticle, a liposome, an exosome, or a cell penetrating peptide.

In some embodiments, the vector comprises one vector, or two or more vectors.

In some aspects, provided herein are pluralities of vectors that comprise any of the vectors described herein, and one or more additional vectors comprising one or more additional polynucleotides encoding an additional portion or an additional component of any of the DNA-targeting systems described herein, any of the gRNAs described herein, any of the combinations described herein, or any of the fusion proteins described herein, or a portion or a component of any of the foregoing.

Provided are pluralities of vectors, that include: a first vector comprising any of the polynucleotides described herein; and a second vector comprising any of the polynucleotides described herein. Also provided herein are pluralities of vectors, comprising: a first vector comprising a polynucleotide encoding a first DNA-targeting system, a first Cas protein and/or a first gRNA of any of the DNA-targeting system described herein or any of the combinations of gRNAs described herein; and; a second vector comprising a polynucleotide encoding a second DNA-targeting system, a second Cas protein and/or a second gRNA of any of the DNA-targeting system described herein or any of the combinations of gRNAs described herein.

In some embodiments, polynucleotides can be cloned into a suitable vector, such as an expression vector or vectors. The expression vector can be any suitable recombinant expression vector, and can be used to transform or transfect any suitable cell. Suitable vectors include those designed for propagation and expansion or for expression or both, such as plasmids and viruses.

In some embodiments, the vector can be a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some embodiments, animal expression vectors include pEUK-C1, pMAM and pMAMneo (Clontech). In some embodiments, a viral vector is used, such as a lentiviral or retroviral vector. In some embodiments, the recombinant expression vectors can be prepared using standard recombinant DNA techniques. In some embodiments, vectors can contain regulatory sequences, such as transcription and translation initiation and termination codons, which are specific to the type of host into which the vector is to be introduced, as appropriate and taking into consideration whether the vector is DNA- or RNA-based. In some embodiments, the vector can contain a nonnative promoter operably linked to the nucleotide sequence encoding the recombinant receptor. In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other promoters known to a skilled artisan also are contemplated.

In some embodiments, recombinant nucleic acids are transferred into cells using recombinant infectious virus particles, such as, e.g., vectors derived from simian virus 40 (SV40), adenoviruses, or adeno-associated virus (AAV). In some embodiments, recombinant nucleic acids are transferred into cells (e.g. central nervous system cells, such as neurons) using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3: 102-109.

In some embodiments, the vector is a lentiviral vector. In some embodiments, the lentiviral vector is an integrase-deficient lentiviral vector. In some embodiments, the lentiviral vector is a recombinant lentiviral vector. In some embodiments, the lentivirus is selected or engineered for a desired tropism (e.g. for central nervous system tropism, or tropism for a heart cell, such as a cardiomyocyte, a skeletal muscle cell, a nervous system cell, such as a neuron, a fibroblast, or an induced pluripotent stem cell). In some embodiments, the cell for any of the provided compositions, such as DNA-targeting systems, fusion proteins, gRNAs, polynucleotides and/or vectors to be delivered is a heart cell, a skeletal muscle cell, a nervous system cell, or an induced pluripotent stem cell. Methods of lentiviral production, transduction, and engineering are known, for example as described in Kasaraneni, N. et al. Sci. Rep. 8(1): 10990 (2018), Ghalch, H. E. G. et al. Biomed. Pharmacother. 128:110276 (2020), and Milone, M. C. et al. Leukemia. 32(7): 1529-1541 (2018). Additional methods for lentiviral transduction are described, for example in Wang et al. (2012) J. Immunother. 35(9): 689-701; Cooper et al. (2003) Blood. 101: 1637-1644; Verhocyen et al. (2009) Methods Mol Biol. 506: 97-114; and Cavalieri et al. (2003) Blood. 102(2): 497-505.

In some embodiments, recombinant nucleic acids are transferred into cells (e.g. central nervous system cells, such as neurons, or a heart cell, a skeletal muscle cell, a nervous system cell, or an induced pluripotent stem cell) via electroporation (see, e.g., Chicaybam et al, (2013) PLOS ONE 8(3): e60298 and Van Tedeloo et al. (2000) Gene Therapy 7(16): 1431-1437). In some embodiments, recombinant nucleic acids are transferred into cells via transposition (see, e.g., Manuri et al. (2010) Hum Gene Ther 21(4): 427-437; Sharma et al. (2013) Molec Ther Nucl Acids 2, c74; and Huang et al. (2009) Methods Mol Biol 506: 115-126). Other methods of introducing and expressing genetic material into immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

### 1. AAV Vectors

In some embodiments, the viral vector is an AAV vector. In some embodiments, the AAV vector is selected from among an AAV1, AAV2, AAV3, AAV4, AAV5, AAV6, AAV7, AAV8, AAV9, AAV10, AAV11, AAV12, or an AAV-DJ vector. In some embodiments, the AAV vector is an AAV vector engineered for central nervous system (CNS) tropism. In some embodiments, the AAV vector is selected from among an AAV1, AAV2, AAV3, AAV4, AAV5, AAV6, AAV7, AAV8, or AAV9 vector. In some embodiments, the AAV vector is an AAV5 vector or an AAV9 vector. In some aspects, the AAV vector is an AAV9 vector. In some aspects, the AAV vector is an AAV5 vector. In some aspects, the AAV vector is an AAV-DJ vector.

In some embodiments, the AAV is selected or engineered for a desired tropism (e.g. for central nervous system tropism, or tropism for a heart cell, such as a cardiomyocyte, a skeletal muscle cell, a nervous system cell, such as a neuron, a fibroblast, or an induced pluripotent stem cell (iPSC)). In some embodiments, the AAV is exhibits tropism for a cardiomyocyte. In some embodiments, the AAV is exhibits tropism for a nervous system cell. In some embodiments, the AAV is exhibits tropism for a cell of the central nervous system (CNS). In some embodiments, the AAV is exhibits tropism for a neuron. In some embodiments, the AAV is exhibits tropism for a fibroblast. In some embodiments, the AAV is exhibits tropism for an iPSC.

In some aspects, nucleic acids or polynucleotides encoding any of the DNA-targeting systems, guide RNAs, fusion proteins, or components, portions or combinations thereof can be delivered to cells or subjects using gene delivery vectors, such as viral vectors. In some aspects, provided herein are viral vectors that comprise any of the nucleic acids or polynucleotides described herein, any of the pluralities of nucleic acids or polynucleotides described herein, or a first polynucleotide or a second polynucleotide of any of the pluralities of polynucleotides described herein, or a portion or a component of any of the foregoing.

Examples of virions that can be employed to deliver any of the nucleic acids or polynucleotides provided herein include but are not limited to retroviral virions, lentiviral virions, adenovirus virions, herpes virus virions, alphavirus virions, and adeno-associated virus (AAV) virions. AAV is a 4.7 kb, single-stranded DNA virus. Recombinant virions based on AAV (rAAV virions) are associated with excellent clinical safety, since wild-type AAV is nonpathogenic and has no etiologic association with any known diseases. In addition, AAV offers the capability for highly efficient delivery and sustained expression of the delivered nucleic acid, composition or component thereof, in numerous tissues, including the nervous system, eye, muscle, lung and brain.

A “recombinant AAV vector (recombinant adeno-associated viral vector)” in some aspects refers to a polynucleotide vector comprising one or more heterologous sequences (i.e., nucleic acid sequence not of AAV origin) that are flanked by at least one AAV inverted terminal repeat sequences (ITR). In some aspects, the recombinant nucleic acid is flanked by two inverted terminal repeat sequences (ITRs). Such recombinant viral vectors can be replicated and packaged into infectious viral particles when present in a host cell that has been infected with a suitable helper virus (or that is expressing suitable helper functions) and that is expressing AAV rep and cap gene products (i.e., AAV Rep and Cap proteins). When a recombinant viral vector is incorporated into a larger polynucleotide (e.g., in a chromosome or in another vector such as a plasmid used for cloning or transfection), then the recombinant viral vector may be referred to as a “pro-vector” which can be “rescued” by replication and encapsidation in the presence of AAV packaging functions and suitable helper functions. A recombinant viral vector can be in any of a number of forms, including, but not limited to, plasmids, linear artificial chromosomes, complexed with lipids, encapsulated within liposomes, and encapsidated in a viral particle, for example, an AAV particle. A recombinant viral vector can be packaged into an AAV virus capsid to generate a “recombinant adeno-associated viral particle (recombinant viral particle)”.

An “rAAV virus” or “rAAV viral particle” refers to a viral particle composed of at least one AAV capsid protein and an encapsidated rAAV vector genome.

“AAV helper functions” refer to functions that allow AAV to be replicated and packaged by a host cell for producing viruses. AAV helper functions can be provided in any of a number of forms, including, but not limited to, helper virus or helper virus genes which aid in AAV replication and packaging. Other AAV helper functions are known, such as genotoxic agents.

A “helper virus” for AAV refers to a virus that allows AAV (which is a defective parvovirus) to be replicated and packaged by a host cell for producing viruses. A helper virus provides “helper functions” which allow for the replication of AAV. A number of such helper viruses have been identified, including adenoviruses, herpesviruses, poxviruses such as vaccinia and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Examples of adenovirus helper functions for the replication of AAV include E1A functions, E1B functions, E2A functions, VA functions and E4orf6 functions. Baculoviruses available from depositories include Autographa californica nuclear polyhedrosis virus.

A preparation of rAAV is said to be “substantially free” of helper virus if the ratio of infectious AAV particles to infectious helper virus particles is at least about 102:1; at least about 104:1, at least about 106:1; or at least about 108:1 or more. In some aspects, preparations are also free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2 and VP3).

In some aspects, the recombinant viral particles for delivery of any of the provided nucleic acids, compositions or components thereof comprise a self-complementary AAV (scAAV) genome. In some aspects, the recombinant AAV genome comprises a first heterologous polynucleotide sequence (e.g., coding strand) and a second heterologous polynucleotide sequence (e.g., the noncoding or antisense strand) wherein the first heterologous polynucleotide sequence can form intrastrand base pairs with the second polynucleotide sequence along most or all of its length. In some aspects, the first heterologous polynucleotide sequence and a second heterologous polynucleotide sequence are linked by a sequence that facilitates intrastrand base-pairing; e.g., a hairpin DNA structure. Hairpin structures are known, for example in siRNA molecules. In some aspects, the first heterologous polynucleotide sequence and a second heterologous polynucleotide sequence are linked by a mutated ITR. In some aspects, the scAAV viral particles comprise a monomeric form of an scAAV genome. In some aspects, the scAAV viral particles comprise the dimeric form of and scAAV genome. In some aspects, AUC as described herein is used to detect the presence of rAAV particles comprising the monomeric form of an scAAV genome. In some aspects, AUC as described herein is used to detect the presence of rAAV particles comprising the dimeric form of an scAAV genome. In some aspects, the packaging of scAAV genomes into capsid is monitored by AUC.

In some aspects, the rAAV particles comprise an AAV1 capsid, an AAV2 capsid, an AAV3 capsid, an AAV4 capsid, an AAV5 capsid, an AAV6 capsid (e.g., a wild-type AAV6 capsid, or a variant AAV6 capsid such as ShH10, as described in US 2012/0164106), an AAV7 capsid, an AAV8 capsid, an AAVrh8 capsid, an AAVrh8R, an AAV9 capsid (e.g., a wild-type AAV9 capsid, or a modified AAV9 capsid as described in US 2013/0323226), an AAV10 capsid, an AAVrh10 capsid, an AAV11 capsid, an AAV12 capsid, a tyrosine capsid mutant, a heparin binding capsid mutant, an AAV2R471A capsid, an AAVAAV2/2-7m8 capsid, an AAV-DJ capsid (e.g., an AAV-DJ/8 capsid, an AAV-DJ/9 capsid, or any other AAV-DJ capsid, such as any of the capsids described, for example, in US 2012/0066783 or Mao, Y. et al., BMC Biotechnol. 16:1 (2016)), an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, a mouse AAV capsid, or an AAV capsid described in U.S. Pat. No. 8,283,151 or WO 2003/042397. In some of the above embodiments described herein, the rAAV particles comprise at least one AAV1 ITR, AAV2 ITR, AAV3 ITR, AAV4 ITR, AAV5 ITR, AAV6 ITR, AAV7 ITR. AAV8 ITR, AAVrh8 ITR, AAV9 ITR, AAV10 ITR, AAVrh10 ITR, AAV11 ITR, AAV12 ITR, AAV-DJ ITR, goat AAV ITR, bovine AAV ITR, or mouse AAV ITR. In some aspects, the rAAV particles comprise ITRs from one AAV serotype and AAV capsid from another serotype. For example, the rAAV particles may comprise the nucleic acid to be delivered (e.g., encoding any of the DNA-targeting systems, fusion proteins, gRNA, compositions or components thereof) flanked by at least one AAV2 ITR encapsidated into an AAV9 capsid. Such combinations may be referred to as pseudotyped rAAV particles. Exemplary AAV vectors include those described, for example, in WO 2020/113034, US 20220001028, US 20220001028, US 20210317474, and US 20160097061.

In some aspects, the viral particle is a recombinant AAV particle comprising a nucleic acid to be delivered flanked by one or two ITRs. The nucleic acid is encapsidated in the AAV particle. The AAV particle also comprises capsid proteins. In some aspects, the nucleic acid comprises the protein coding sequence or RNA-expressing sequences to be delivered (e.g., any of the DNA-targeting systems, fusion proteins, gRNA, compositions or components thereof) operatively linked components in the direction of transcription, control sequences including transcription initiation and termination sequences, thereby forming an expression cassette. The expression cassette is flanked on the 5′ and 3′ end by at least one functional AAV ITR sequences. By “functional AAV ITR sequences” it is meant that the ITR sequences function as intended for the rescue, replication and packaging of the AAV virion. See Davidson et al., PNAS, 2000, 97(7)3428-32; Passini et al., J. Virol., 2003, 77(12):7034-40; and Pechan et al., Gene Ther., 2009, 16:10-16, all of which are incorporated herein in their entirety by reference. For practicing some aspects of the invention, the recombinant vectors comprise at least all of the sequences of AAV essential for encapsidation and the physical structures for infection by the rAAV. AAV ITRs for use in the vectors of the invention need not have a wild-type nucleotide sequence (e.g., as described in Kotin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified. Sec Gao et al., PNAS, 2002, 99(18): 11854-6; Gao et al., PNAS, 2003, 100(10):6081-6; and Bossis et al., J. Virol., 2003, 77(12):6799-810. Use of any AAV serotype is considered within the scope of the present invention. In some aspects, a rAAV vector is a vector derived from an AAV serotype, including without limitation, AAV1, AAV2, AAV3, AAV4, AAV5, AAV6, AAV7, AAV8, AAV9, AAVrh.8, AAVrh.10, AAV11, AAV12, a tyrosine capsid mutant, a heparin binding capsid mutant, an AAV2R471A capsid, an AAVAAV2/2-7m8 capsid, an AAV-DJ capsid, an AAV2 N587A capsid, an AAV2 E548A capsid, an AAV2 N708A capsid, an AAV V708K capsid, a goat AAV capsid, an AAV1/AAV2 chimeric capsid, a bovine AAV capsid, or a mouse AAV capsid, or the like. In some aspects, the nucleic acid in the AAV comprises an ITR of AAV1, AAV2, AAV3, AAV4, AAV5, AAV6, AAV7, AAV8, AAV9, AAVrh.8, AAVrh10, AAV11, AAV12 or the like. In further embodiments, the rAAV particle comprises capsid proteins of AAV1, AAV2, AAV3, AAV4, AAV5, AAV6, AAV7, AAV8, AAV9, AAVrh.8, AAVrh. 10, AAV11, AAV12 or the like. In further embodiments, the rAAV particle comprises capsid proteins of an AAV serotype from Clades A-F (Gao, et al. J. Virol. 2004, 78(12):6381).

Different AAV serotypes are used to optimize transduction of particular target cells or to target specific cell types within a particular target tissue (e.g., a diseased tissue). A rAAV particle can comprise viral proteins and viral nucleic acids of the same serotype or a mixed serotype. For example, a rAAV particle can comprise AAV9 capsid proteins and at least one AAV2 ITR or it can comprise AAV2 capsid proteins and at least one AAV9 ITR. In yet another example, a rAAV particle can comprise capsid proteins from both AAV9 and AAV2, and further comprise at least one AAV2 ITR. Any combination of AAV serotypes for production of a rAAV particle is provided herein as if each combination had been expressly stated herein.

In some aspects, the AAV comprises at least one AAV1 ITR and capsid protein from any of AAV-DJ, AAV2, AAV3, AAV4, AAV5, AAV6, AAV7, AAV8, AAV9, AAVrh.8, AAVrh10, AAV11, and/or AAV12. In some aspects, the AAV comprises at least one AAV2 ITR and capsid protein from any of AAV-DJ, AAV1, AAV3, AAV4, AAV5, AAV6, AAV7, AAV8, AAV9, AAVrh.8, AAVrh10, AAV11, and/or AAV12. In some aspects, the AAV comprises at least one AAV3 ITR and capsid protein from any of AAV-DJ, AAV1, AAV2, AAV4, AAV5, AAV6, AAV7, AAV8, AAV9, AAVrh.8, AAVrh10, AAV11, and/or AAV12. In some aspects, the AAV comprises at least one AAV4 ITR and capsid protein from any of AAV-DJ, AAV1, AAV2, AAV3, AAV5, AAV6, AAV7, AAV8, AAV9, AAVrh.8, AAVrh10, AAV11, and/or AAV12. In some aspects, the AAV comprises at least one AAV5 ITR and capsid protein from any of AAV-DJ, AAV1, AAV2, AAV3, AAV4, AAV6, AAV7, AAV8, AAV9, AAVrh.8, AAVrh10, AAV11, and/or AAV12. In some aspects, the AAV comprises at least one AAV6 ITR and capsid protein from any of AAV-DJ, AAV1, AAV2, AAV3, AAV4, AAV5, AAV7, AAV8, AAV9, AAVrh.8, AAVrh10, AAV11, and/or AAV12. In some aspects, the AAV comprises at least one AAV7 ITR and capsid protein from any of AAV-DJ, AAV1, AAV2, AAV3, AAV4, AAV5, AAV6, AAV8, AAV9, AAVrh.8, AAVrh10, AAV11, and/or AAV12. In some aspects, the AAV comprises at least one AAV8 ITR and capsid protein from any of AAV-DJ, AAV1, AAV2, AAV3, AAV4, AAV5, AAV6, AAV7, AAV9, AAVrh.8, AAVrh10, AAV11, and/or AAV12. In some aspects, the AAV comprises at least one AAV9 ITR and capsid protein from any of AAV-DJ, AAV1, AAV2, AAV3, AAV4, AAV5, AAV6, AAV7, AAV8, AAVrh.8, AAVrh10, AAV11, and/or AAV12. In some aspects, the AAV comprises at least one AAVrh8 ITR and capsid protein from any of AAV-DJ, AAV1, AAV2, AAV3, AAV4, AAV5, AAV6, AAV8, AAV9, AAVrh10, AAV11, and/or AAV12. In some aspects, the AAV comprises at least one AAVrh10 ITR and capsid protein from any of AAV-DJ, AAV1, AAV2, AAV3, AAV4, AAV5, AAV6, AAV7, AAV8, AAV9, AAV11, and/or AAV12. In some aspects, the AAV comprises at least one AAV11 ITR and capsid protein from any of AAV-DJ, AAV1, AAV2, AAV3, AAV4, AAV5, AAV6, AAV7, AAV8, AAVrh8, AAV9, AAVrh10, and/or AAV12. In some aspects, the AAV comprises at least one AAV12 ITR and capsid protein from any of AAV-DJ, AAV1, AAV2, AAV3, AAV4, AAV5, AAV6, AAV7, AAV8, AAV rh8, AAV9, AAVrh10, and/or AAV11. In some aspects, the AAV comprises at least one AAV-DJ ITR and capsid protein from any of AAV1, AAV2, AAV3, AAV4, AAV5, AAV6, AAV7, AAV8, AAV rh8, AAV9, AAVrh10, and/or AAV11.

In some aspects, the viral particles comprise a recombinant self-complementing genome. AAV viral particles with self-complementing genomes and methods of use of self-complementing AAV genomes are described in U.S. Pat. Nos. 6,596,535; 7,125,717; 7,765,583; 7,785,888; 7,790,154; 7,846,729; 8,093,054; and 8,361,457; and Wang Z., et al., (2003) Gene Ther 10:2105-2111, each of which are incorporated herein by reference in its entirety. A rAAV comprising a self-complementing genome will quickly form a double stranded DNA molecule by virtue of its partially complementing sequences (e.g., complementing coding and non-coding strands). In some aspects, an AAV viral particle comprises an AAV genome, wherein the rAAV genome comprises a first heterologous polynucleotide sequence (e.g., a coding strand) and a second heterologous polynucleotide sequence (e.g., the noncoding or antisense strand) wherein the first heterologous polynucleotide sequence can form intrastrand base pairs with the second polynucleotide sequence along most or all of its length. In some aspects, the first heterologous polynucleotide sequence and a second heterologous polynucleotide sequence are linked by a sequence that facilitates intrastrand base-pairing; e.g., a hairpin DNA structure. Hairpin structures include, for example in siRNA molecules. In some aspects, the first heterologous polynucleotide sequence and a second heterologous polynucleotide sequence are linked by a mutated ITR (e.g., the right ITR). The mutated ITR comprises a deletion of the D region comprising the terminal resolution sequence. As a result, on replicating an AAV viral genome, the rep proteins will not cleave the viral genome at the mutated ITR and as such, a recombinant viral genome comprising the following in 5′ to 3′ order will be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR.

Methods for production of rAAV vectors, including transfection, stable cell line production, and infectious hybrid virus production systems which include adenovirus-AAV hybrids, herpesvirus-AAV hybrids (Conway, J E et al., (1997) J. Virology 71(11):8780-8789) and baculovirus-AAV hybrids can be employed. Typically, rAAV production cultures for the production of rAAV virus particles all require; 1) suitable host cells, including, for example, human-derived cell lines such as HeLa, A549, or 293 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a nucleic acid to be delivered (such as any of the DNA-targeting systems, fusion proteins, compositions or components thereof) flanked by at least one AAV ITR sequences; and 5) suitable media and media components to support rAAV production. In some aspects, the AAV rep and cap gene products may be from any AAV serotype. In general, but not obligatory, the AAV rep gene product is of the same serotype as the ITRs of the rAAV vector genome as long as the rep gene products may function to replicated and package the rAAV genome. Suitable media may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551. In some aspects, the AAV helper functions are provided by adenovirus or HSV. In some aspects, the AAV helper functions are provided by baculovirus and the host cell is an insect cell (e.g., Spodoptera frugiperda (Sf9) cells).

Suitable rAAV production culture media of the present invention may be supplemented with serum or serum-derived recombinant proteins at a level of 0.5%-20% (v/v or w/v). Alternatively, rAAV vectors may be produced in serum-free conditions which may also be referred to as media with no animal-derived products. Commercial or custom media designed to support production of rAAV vectors may also be supplemented with one or more cell culture components, including without limitation glucose, vitamins, amino acids, and or growth factors, in order to increase the titer of rAAV in production cultures.

rAAV production cultures can be grown under a variety of conditions (over a wide temperature range, for varying lengths of time, and the like) suitable to the particular host cell being utilized. rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa, 293, and SF-9 cells which can be cultured in a variety of ways including, for example, spinner flasks, stirred tank bioreactors, and disposable systems such as the Wave bag system.

rAAV vector particles of the invention may be harvested from rAAV production cultures by lysis of the host cells of the production culture or by harvest of the spent media from the production culture, provided the cells are cultured under conditions to cause release of rAAV particles into the media from intact cells, as described in U.S. Pat. No. 6,566,118). Suitable methods of lysing cells include for example multiple freeze/thaw cycles, sonication, microfluidization, and treatment with chemicals, such as detergents and/or proteases.

In some aspects, recombinant viral particles for delivery of the nucleic acids, compositions or components thereof are highly purified, suitably buffered, and concentrated. In some aspects, the viral particles are concentrated to at least about 1×10⁷ vg/mL to about 9×10¹³ vg/mL or any concentration therebetween.

In some aspects, adeno-associated virus (AAV)-based vectors are generally used vector system for neurologic gene therapy, with an excellent safety record established in multiple clinical trials (Kaplitt et al., (2007) Lancet 369:2097-2105; Eberling et al., (2008) Neurology 70:1980-1983; Fiandaca et al., (2009) Neuroimage 47 Suppl. 2:T27-35). In some cases, effective treatment of neurologic disorders has been hindered by problems associated with the delivery of AAV vectors to affected cell populations. This delivery issue has been especially problematic for disorders involving the cerebral cortex. Simple injections do not distribute AAV vectors effectively, relying on diffusion, which is effective only within a 1- to 3-mm radius. An alternative method, convection-enhanced delivery (CED) (Nguyen et al., (2003) J. Neurosurg. 98:584-590), has been used clinically in gene therapy (AAV2-hAADC) for Parkinson's disease (Fiandaca et al., (2008) Exp. Neurol. 209:51-57). The underlying principle of CED involves pumping infusate into brain parenchyma under sufficient pressure to overcome the hydrostatic pressure of interstitial fluid, thereby forcing the infused particles into close contact with the dense perivasculature of the brain. Pulsation of these vessels acts as a pump, distributing the particles over large distances throughout the parenchyma (Hadaczek et al., (2006) Hum. Gene Ther. 17:291-302). To increase the safety and efficacy of CED a reflux-resistant cannula (Krauze et al., (2009) Methods Enzymol. 465:349-362) can be employed along with monitored delivery with real-time MRI. Monitored delivery allows for the quantification and control of aberrant events, such as cannula reflux and leakage of infusate into ventricles (Eberling et al., (2008) Neurology 70:1980-1983; Fiandaca et al., (2009) Neuroimage 47 Suppl. 2:T27-35; Saito et al., (2011) Journal of Neurosurgery Pediatrics 7:522-526).

In some aspects, the nucleic acid to be delivered is operably linked to a promoter. In some aspects, the promoter expresses the nucleic acid to be delivered in a cell of the CNS. In some aspects, the promoter expresses the nucleic acid to be delivered in a brain cell. In some aspects, the promoter expresses the nucleic acid to be delivered in a neuron and/or a glial cell. In some aspects, the neuron is a medium spiny neuron of the caudate nucleus, a medium spiny neuron of the putamen, a neuron of the cortex layer IV and/or a neuron of the cortex layer V. In some aspects, the glial cell is an astrocyte. In some aspects, the promoter is a CBA promoter, a minimum CBA promoter, a CMV promoter or a GUSB promoter. In some aspects, the promoter is inducible. In further embodiments, the rAAV vector comprises one or more of an enhancer, a splice donor/splice acceptor pair, a matrix attachment site, or a polyadenylation signal.

In some aspects, the methods for delivering a recombinant adeno-associated viral (rAAV) particle to the central nervous system of a subject involve administering the rAAV particle to the striatum, wherein the rAAV particle comprises a rAAV vector encoding a nucleic acid to be delivered that is expressed in at least the cerebral cortex and striatum of the subject. In some aspects, methods for delivering a rAAV particle to the central nervous system of a subject involve administering the rAAV particle to the striatum, wherein the rAAV particle comprises an rAAV vector encoding a nucleic acid to be delivered that is expressed in at least the cerebral cortex and striatum of the subject and wherein the rAAV particle comprises an AAV serotype 1 (AAV1) capsid. In some aspects, methods for delivering a rAAV particle to the central nervous system of a subject comprise administering the rAAV particle to the striatum, wherein the rAAV particle comprises an rAAV vector encoding a nucleic acid to be delivered that is expressed in at least the cerebral cortex and striatum of the subject and wherein the rAAV particle comprises an AAV serotype 2 (AAV2) capsid. In some aspects, methods for treating a central nervous system-related disease in a subject involve administering a rAAV particle to the striatum, wherein the rAAV particle comprises a rAAV vector encoding a nucleic acid to be delivered that is expressed in at least the cerebral cortex and striatum of the subject. In some aspects, the subject is a human.

In some aspects, a rAAV particle is administered to one or more regions of the central nervous system (CNS). In some aspects, the rAAV particle is administered to the striatum. The striatum is known as a region of the brain that receives inputs from the cerebral cortex (the term “cortex” may be used interchangeably herein) and sends outputs to the basal ganglia (the striatum is also referred to as the striate nucleus and the neostriatum). In some aspects, the striatum controls both motor movements and emotional control/motivation and has been implicated in many neurological diseases, such as Huntington's disease. Several cell types of interest are located in the striatum, including without limitation spiny projection neurons (also known as medium spiny neurons), GABAergic interneurons, and cholinergic interneurons. Medium spiny neurons make up most of the striatal neurons. These neurons are GABAergic and express dopamine receptors. Each hemisphere of the brain contains a striatum.

In some aspects, important substructures of the striatum include the caudate nucleus and the putamen. In some aspects, the rAAV particle is administered to the caudate nucleus (the term “caudate” may be used interchangeably herein). The caudate nucleus is known as a structure of the dorsal striatum. The caudate nucleus has been implicated in control of functions such as directed movements, spatial working memory, memory, goal-directed actions, emotion, sleep, language, and learning. Each hemisphere of the brain contains a caudate nucleus.

In some aspects, the rAAV particle is administered to the putamen. Along with the caudate nucleus, the putamen is known as a structure of the dorsal striatum. The putamen comprises part of the lenticular nucleus and connects the cerebral cortex with the substantia nigra and the globus pallidus. Highly integrated with many other structures of the brain, the putamen has been implicated in control of functions such as learning, motor learning, motor performance, motor tasks, and limb movements. Each hemisphere of the brain contains a putamen.

In some aspects, rAAV particles may be administered to one or more sites of the striatum. In some aspects, the rAAV particle is administered to the putamen and the caudate nucleus of the striatum. In some aspects, the rAAV particle is administered to the putamen and the caudate nucleus of each hemisphere of the striatum. In some aspects, the rAAV particle is administered to at least one site in the caudate nucleus and two sites in the putamen.

In some aspects, the rAAV particle is administered to one hemisphere of the brain. In some aspects, the rAAV particle is administered to both hemispheres of the brain. For example, in some aspects, the rAAV particle is administered to the putamen and the caudate nucleus of each hemisphere of the striatum. In some aspects, the composition containing rAAV particles is administered to the striatum of each hemisphere. In some aspects, the composition containing rAAV particles is administered to striatum of the left hemisphere or the striatum of the right hemisphere and/or the putamen of the left hemisphere or the putamen of the right hemisphere. In some aspects, the composition containing rAAV particles is administered to any combination of the caudate nucleus of the left hemisphere, the caudate nucleus of the right hemisphere, the putamen of the left hemisphere and the putamen of the right hemisphere.

In some aspects, the methods involving administration to CNS an effective amount of recombinant viral particles to the striatum can be employed for delivery, wherein the rAAV particle comprises a rAAV vector encoding a nucleic acid to be delivered that is expressed in at least the cerebral cortex and striatum. In some aspects, the viral titer of the rAAV particles is at least about any of 5×10¹², 6×10¹², 7×10¹², 8×10¹², 9×10¹², 10×10¹², 11×10¹², 15×10¹², 20×10¹², 25×10¹², 30×10¹², or 50×10¹² genome copies/mL. In some aspects, the viral titer of the rAAV particles is about any of 5×10¹² to 6×10¹², 6×10¹² to 7×10¹², 7×10¹² to 8×10¹², 8×10¹² to 9×10¹², 9×10¹² to 10×10¹², 10×10¹² to 11×10¹², 11×10¹² to 15×10¹², 15×10¹² to 20×10¹², 20×10¹² to 25×10¹², 25×10¹² to 30×10¹², 30×10¹² to 50×10¹², or 50×10¹² to 10×10¹² genome copies/mL. In some aspects, the viral titer of the rAAV particles is about any of 5×10¹² to 10×10¹², 10×10¹² to 25×10¹², or 25×10¹² to 50×10¹² genome copies/mL. In some aspects, the viral titer of the rAAV particles is at least about any of 5×10⁹. 6×10⁹, 7×10⁹, 8×10⁹, 9×10⁹, 10×10⁹, 11×10⁹, 15×10⁹, 20×10⁹, 25×10⁹, 30×10⁹, or 50×10⁹ transducing units/mL. In some aspects, the viral titer of the rAAV particles is about any of 5×10⁹ to 6×10⁹, 6×10⁹ to 7×10⁹, 7×10⁹ to 8×10⁹, 8×10⁹ to 9×10⁹, 9×10⁹ to 10×10⁹, 10×10⁹ to 11×10⁹, 11×10⁹ to 15×10⁹, 15×10⁹ to 20×10⁹, 20×10⁹ to 25×10⁹, 25×10⁹ to 30×10⁹, 30×10⁹ to 50×10⁹ or 50×10⁹ to 100×10⁹ transducing units/mL. In some aspects, the viral titer of the rAAV particles is about any of 5×10⁹ to 10×10⁹, 10×10⁹ to 15×10⁹, 15×10⁹ to 25×10⁹, or 25×10⁹ to 50×10⁹ transducing units/mL. In some aspects, the viral titer of the rAAV particles is at least any of about 5×10¹⁰, 6×10¹⁰, 7×10¹⁰, 8×10¹⁰, 9×10¹⁰, 10×10¹⁰, 11×10¹⁰, 15×10¹⁰, 20×10¹⁰, 25×10¹⁰, 30×10¹⁰, 40×10¹⁰, or 50×10¹⁰ infectious units/mL. In some aspects, the viral titer of the rAAV particles is at least any of about 5×10¹⁰ to 6×10¹⁰, 6×10¹⁰ to 7×10¹⁰, 7×10¹⁰ to 8×10¹⁰, 8×10¹⁰ to 9×10¹⁰, 9×10¹⁰ to 10×10¹⁰, 10×10¹⁰ to 11×10¹⁰, 11×10¹⁰ to 15×10¹⁰, 15×10¹⁰ to 20×10¹⁰, 20×10¹⁰ to 25×10¹⁰, 25×10¹⁰ to 30×10¹⁰, 30×10¹⁰ to 40×10¹⁰, 40×10¹⁰ to 50×10¹⁰, or 50×10¹⁰ to 100×10¹⁰ infectious units/mL. In some aspects, the viral titer of the rAAV particles is at least any of about 5×10¹⁰ to 10×10¹⁰, 10×10¹⁰ to 15×10¹⁰, 15×10¹⁰ to 25×10¹⁰, or 25×10¹⁰ to 50×10¹⁰ infectious units/mL.

In some aspects, an effective amount of recombinant viral particles is administered to the striatum, wherein the rAAV particle comprises a rAAV vector encoding a nucleic acid to be delivered that is expressed in at least the cerebral cortex and striatum. In some aspects, the dose of viral particles administered to the individual is at least about any of 1×10⁸ to about 1×10¹³ genome copies/kg of body weight. In some aspects, the dose of viral particles administered to the individual is about 1×10⁸ to 1×10¹³ genome copies/kg of body weight.

In some aspects, an effective amount of recombinant viral particles is administered to the striatum, wherein the rAAV particle comprises a rAAV vector encoding a nucleic acid to be delivered that is expressed in at least the cerebral cortex and striatum. In some aspects, the total amount of viral particles administered to the individual is at least about 1×10⁹ to about 1×10¹⁴ genome copies. In some aspects, the total amount of viral particles administered to the individual is about 1×10⁹ to about 1×10¹⁴ genome copies.

### 2. Non-Viral Vectors

In some embodiments, the vector is a non-viral vector. In some aspects, exemplary non-viral vectors include polymers, lipids, peptides, inorganic materials, and hybrid systems. In some aspects, the non-viral vector is a lipid nanoparticle (LNP), a liposome, an exosome, or a cell penetrating peptide. In some aspects, the non-viral vector is a lipid nanoparticle (LNP). In some aspects, the LNP can be used for delivery to the liver. Exemplary non-viral vectors include those described in WO 2020/051561, US 20210301274, Zu et al., The AAPS Journal volume 23, Article number: 78 (2021), and Sung et al., Biomaterials Research volume 23, Article number: 8 (2019), Nyamay' Antu et al., Cell & Gene Therapy Insights 2019; 5(S1):51-57, and Yin et al., Nature Reviews Genetics 15:541-555 (2014).

In some embodiments, the vector is a non-viral vector selected from: a lipid nanoparticle, a liposome, an exosome, or a cell penetrating peptide

In some embodiments, a vector described herein is or comprises a lipid nanoparticle (LNP). In some embodiments, any of the epigenetic-modifying DNA-targeting systems, gRNAs, Cas-gRNA combinations, polynucleotides, fusion proteins, or components thereof described herein, are incorporated in lipid nanoparticles (LNPs), such as for delivery. In some embodiments, the lipid nanoparticle is a vector for delivery. In some embodiments, the nanoparticle may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG and PEGylated lipids. In another aspect, the lipid may be a cationic lipid such as, but not limited to, DLin-DMA, DLin-D-DMA, DLin-MC 3-DMA, DLin-KC2-DMA and DODMA.

Lipid nanoparticles can be used for the delivery of encapsulated or associated (e.g., complexed) therapeutic agents, including nucleic acids and proteins, such as those encoding and/or comprising CRISPR/Cas systems. Sec, e.g., U.S. Pat. Nos. 10,723,692, 10,941,395, and WO 2015/035136.

In some embodiments, the provided methods involve use of a lipid nanoparticle (LNP) comprising mRNA, such as mRNA encoding a protein component of any of the provided DNA-targeting systems, for example any of the fusion proteins provided herein. In some embodiments, the mRNA can be produced using methods known in the art such as in vitro transcription. In some embodiments of the method, the mRNA comprises a 5′ cap. In some embodiments, the 5′ cap is an altered nucleotide on the 5′ end of primary transcripts such as messenger RNA. In some aspects, the 5′ caps of the mRNA improves one or more of RNA stability and processing, mRNA metabolism, the processing and maturation of an RNA transcript in the nucleus, transport of mRNA from the nucleus to the cytoplasm, mRNA stability, and efficient translation of mRNA to protein. In some embodiments, a 5′ cap can be a naturally-occurring 5′ cap or one that differs from a naturally-occurring cap of an mRNA. A 5′ cap may be any 5′ cap known to a skilled artisan. In certain embodiments, the 5′ cap is selected from the group consisting of an Anti-Reverse Cap Analog (ARCA) cap, a 7-methyl-guanosine (7mG) cap, a CleanCap® analog, a vaccinia cap, and analogs thereof. For instance, the 5′ cap may include, without limitation, an anti-reverse cap analogs (ARCA) (U.S. Pat. No. 7,074,596), 7-methyl-guanosine, CleanCap® analogs, such as Cap 1 analogs (Trilink; San Diego, CA), or enzymatically capped using, for example, a vaccinia capping enzyme or the like. In some embodiments, the mRNA may be polyadenylated. The mRNA may contain various 5′ and 3′ untranslated sequence elements to enhance expression of the encoded protein and/or stability of the mRNA itself. Such elements can include, for example, posttranslational regulatory elements such as a woodchuck hepatitis virus post-transcriptional regulatory element (WPRE). In some embodiments, the mRNA comprises at least one nucleoside modification. The mRNA may contain modifications of naturally-occurring nucleosides to nucleoside analogs. Any nucleoside analogs known in the art are envisioned. Such nucleoside analogs can include, for example, those described in U.S. Pat. No. 8,278,036. In certain embodiments of the method, the nucleoside modification is selected from the group consisting of a modification from uridine to pseudouridine and uridine to N1-methyl pseudouridine. In particular embodiments of the method the nucleoside modification is from uridine to pseudouridine.

In some embodiments, LNPs useful for in the present methods comprise a cationic lipid selected from DLin-DMA (1,2-dilinoleyloxy-3-dimethylaminopropane), DLin-MC3-DM A (dilinoleylmethyl-4-dimethylaminobutyrate), DLin-KC2-DMA (2,2-dilinoleyl-4-(2-dimethylaminoethyl)-[1,3]-dioxolane), DODMA (1,2-dioleyloxy-N,N-dimethyl-3-aminopropane), SS-OP (Bis[2-(4-{2-[4-(cis-9 octadecenoyloxy)phenylacetoxy]ethyl} piperidinyl)ethyl] disulfide), and derivatives thereof. DLin-MC3-DMA and derivatives thereof are described, for example, in WO 2010/144740. DODMA and derivatives thereof are described, for example, in U.S. Pat. No. 7,745,651 and Mok et al. (1999), Biochimica et Biophysica Acta, 1419(2): 137-150. DLin-DMA and derivatives thereof are described, for example, in U.S. Pat. No. 7,799,565. DLin-KC2-DMA and derivatives thereof are described, for example, in U.S. Pat. No. 9,139,554. SS-OP (NOF America Corporation, White Plains, NY) is described, for example, at https://www.nofamerica.com/store/index.php?dispatch=products.view&product_id=962. Additional and non-limiting examples of cationic lipids include methylpyridiyl-dialkyl acid (MPDACA), palmitoyl-olcoyl-nor-arginine (PONA), guanidino-dialkyl acid (GUADACA), 1,2-di-O-octadecenyl-3-trimethylammonium propane (DOTMA), 1,2-dioleoyl-3-trimethylammonium-propane (DOTAP), Bis{2-[N-methyl-N-(a-D-tocopherolhemisuccinatepropyl)amino]ethyl} disulfide (SS-33/3AP05), Bis{2-[4-(a-D-tocopherolhemisuccinateethyl)piperidyl] ethyl} disulfide (SS33/4PE15), Bis{2-[4-(cis-9-octadecenoatecthyl)-1-piperidinyl] ethyl} disulfide (SS18/4PE16), and Bis{2-[4-(cis,cis-9,12-octadecadienoateethyl)-1-piperidinyl] ethyl} disulfide (SS18/4PE13). In further embodiments, the lipid nanoparticles also comprise one or more non-cationic lipids and a lipid conjugate.

In some embodiments, the molar concentration of the cationic lipid is from about 20% to about 80%, from about 30% to about 70%, from about 40% to about 60%, from about 45% to about 55%, or about 20%, about 25%, about 30%, about 35%, about 40%, about 45%, about 50%, about 55%, about 60%, about 65%, about 70%, about 75%, or about 80% of the total lipid molar concentration, wherein the total lipid molar concentration is the sum of the cationic lipid, the non-cationic lipid, and the lipid conjugate molar concentrations. In certain embodiments, the lipid nanoparticles comprise a molar ratio of cationic lipid to any of the polynucleotides of from about 1 to about 20, from about 2 to about 16, from about 4 to about 12, from about 6 to about 10, or about 1, about 2, about 3, about 4, about 5, about 6, about 7, about 8, about 9, about 10, about 11, about 12, about 13, about 14, about 15, about 16, about 17, about 18, about 19, or about 20.

In some embodiments, the lipid nanoparticles can comprise at least one non-cationic lipid. In particular embodiments, the molar concentration of the non-cationic lipids is from about 20% to about 80%, from about 30% to about 70%, from about 40% to about 70%, from about 40% to about 60%, from about 46% to about 50%, or about 20%, about 25%, about 30%, about 35%, about 40%, about 45%, about 48.5%, about 50%, about 55%, about 60%, about 65%, about 70%, about 75%, or about 80% of the total lipid molar concentration. Non-cationic lipids include, in some embodiments, phospholipids and steroids.

In some embodiments, phospholipids useful for the lipid nanoparticles described herein include, but are not limited to, 1,2-Distearoyl-sn-glycero-3-phosphocholine (DSPC), 1,2-Didecanoyl-sn-glycero-3-phosphocholine (DDPC), 1,2-Dierucoyl-sn-glycero-3-phosphate(Sodium Salt) (DEPA-NA), 1,2-Dierucoyl-sn-glycero-3-phosphocholine (DEPC), 1,2-Dierucoyl-sn-glycero-3-phosphoethanolamine (DEPE), 1,2-Dierucoyl-sn-glycero-3[Phospho-rac-(1-glycerol)(Sodium Salt) (DEPG-NA), 1,2-Dilinoleoyl-sn-glycero-3-phosphocholine (DLOPC), 1,2-Dilauroyl-sn-glycero-3-phosphate(Sodium Salt) (DLPA-NA), 1,2-Dilauroyl-sn-glycero-3-phosphocholine (DLPC), 1,2-Dilauroyl-sn-glycero-3-phosphoethanolamine (DLPE), 1,2-Dilauroyl-sn-glycero-3[Phospho-rac-(1-glycerol.)(Sodium Salt) (DLPG-NA), 1,2-Dilauroyl-sn-glycero-3[Phospho-rac-(1-glycerol)(Ammonium Salt) (DLPG-NH4), 1,2-Dilauroyl-sn-glycero-3-phosphoserine(Sodium Salt) (DLPS-NA), 1,2-Dimyristoyl-sn-glycero-3-phosphate(SodiumSalt) (DMPA-NA), 1,2-Dimyristoyl-sn-glycero-3-phosphocholine (DMPC), 1,2-Dimyristoyl-sn-glycero-3-phosphoethanolamine (DMPE), 1,2-Dimyristoyl-sn-glycero-3[Phospho-rac-(1-glycerol)(Sodium Salt) (DMPG-NA), 1,2-Dimyristoyl-sn-glycero-3[Phospho-rac-(1-glycerol)(Ammonium Salt) (DMPG-NH4), 1,2-Dimyristoyl-sn-glycero-3[Phospho-rac-(1-glycerol)(Sodium/Ammonium Salt) (DMPG-NH4/NA), 1,2-Dimyristoyl-sn-glycero-3-phosphoserine(Sodium Salt) (DMPS-NA), 1,2-Dioleoyl-sn-glycero-3-phosphate(Sodium Salt) (DOPA-NA), 1,2-Dioleoyl-sn-glycero-3-phosphocholine (DOPC), 1,2-Dioleoyl-sn-glycero-3-phosphoethanolamine (DOPE), 1,2-Dioleoyl-sn-glycero-3[Phospho-rac-(1-glycerol)(Sodium Salt) (DOPG-NA), 1,2-Dioleoyl-sn-glycero-3-phosphoserine(Sodium Salt) (DOPS-NA), 1,2-Dipalmitoyl-sn-glycero-3-phosphate(Sodium Salt) (DPPA-NA), 1,2-Dipalmitoyl-sn-glycero-3-phosphocholine (DPPC), 1,2-Dipalmitoyl-sn-glycero-3-phosphoethanolamine (DPPE), 1,2-Dipalmitoyl-sn-glycero-3[Phospho-rac-(1-glycerol)(Sodium Salt) (DPPG-NA), 1,2-Dipalmitoyl-sn-glycero-3[Phospho-rac-(1-glycerol)(Ammonium Salt) (DPPG-NH4), 1,2-Dipalmitoyl-sn-glycero-3-phosphoserine(Sodium Salt) (DPPS-NA), 1,2-Distearoyl-sn-glycero-3-phosphate(Sodium Salt) (DSPA-NA), 1,2-Distearoyl-sn-glycero-3-phosphoethanolamine (DSPE), 1,2-Distearoyl-sn-glycero-3[Phospho-rac-(1-glycerol)(Sodium Salt) (DSPG-NA), 1,2-Distearoyl-sn-glycero-3[Phospho-rac-(1-glycerol)(Ammonium Salt) (DSPG-NH4), 1,2-Distearoyl-sn-glycero-3-phosphoserine(Sodium Salt) (DSPS-NA), Egg-PC (EPC), Hydrogenated Egg PC (HEPC), Hydrogenated Soy PC (HSPC), 1-Myristoyl-sn-glycero-3-phosphocholine (LY S OPCM YRIS TIC), 1-Palmitoyl-sn-glycero-3-phosphocholine (LYSOPCPALMITIC), 1-Stearoyl-sn-glycero-3-phosphocholine (LYSOPC STEARIC), 1-Myristoyl-2-palmitoyl-sn-glycero3-phosphocholine (MPPC), 1-Myristoyl-2-stearoyl-sn-glycero-3-phosphocholine (MSPC), 1-Palmitoyl-2-myristoyl-sn-glycero-3-phosphocholine (PMPC), I-Palmitoyl-2-olcoyl-sn-glycero-3-phosphocholine (POPC), 1-Palmitoyl-2-oleoyl-sn-glycero-3-phosphoethanolamine (POPE), 1-Palmitoyl-2-oleoyl-sn-glycero-3[Phospho-rac-(1-glycerol)] (Sodium Salt) (POPG-NA), 1-Palmitoyl-2-stearoyl-sn-glycero-3-phosphocholine (PS PC), 1-Stearoyl-2-myristoyl-sn-glycero-3-phosphocholine (SMPC), 1-Stearoyl-2-olcoyl-sn-glycero-3-phosphocholine (SOPC), and 1-Stearoyl-2-palmitoyl-sn-glycero-3-phosphocholine (SPPC). In particular embodiments, the phospholipid is DSPC. In particular embodiments, the phospholipid is DOPE. In particular embodiments, the phospholipid is DOPC.

In some embodiments, the non-cationic lipids comprised by the lipid nanoparticles include one or more steroids. Steroids useful for the lipid nanoparticles described herein include, but are not limited to, cholestanes such as cholesterol, cholanes such as cholic acid, pregnanes such as progesterone, androstanes such as testosterone, and estranes such as estradiol. Further steroids include, but are not limited to, cholesterol (ovine), cholesterol sulfate, desmosterol-d6, cholesterol-d7, lathosterol-d7, desmosterol, stigmasterol, lanosterol, dehydrocholesterol, dihydrolanosterol, zymosterol, lathosterol, zymosterol-d5, 14-demethyl-lanosterol, 14-demethyl-lanosterol-d6, 8(9)-dehydrocholesterol, 8(14)-dehydrocholesterol, diosgenin, DHEA sulfate, DHEA, lanosterol-d6, dihydrolanosterol-d7, campesterol-d6, sitosterol, lanosterol-95, Dihydro FF-MAS-d6, zymostenol-d7, zymostenol, sitostanol, campestanol, campesterol, 7-dehydrodesmosterol, pregnenolone, sitosterol-d7, Dihydro T-MAS, Delta 5-avenasterol, Brassicasterol, Dihydro FF-MAS, 24-methylene cholesterol, cholic acid derivatives, cholesteryl esters, and glycosylated sterols. In particular embodiments, the lipid nanoparticles comprise cholesterol.

In some embodiments, the lipid nanoparticles comprise a lipid conjugate. Such lipid conjugates include, but are not limited to, ceramide PEG derivatives such as C8 PEG2000 ceramide, C16 PEG2000 ceramide, C8 PEG5000 ceramide, C16 PEG5000 ceramide, C8 PEG750 ceramide, and C16 PEG750 ceramide, phosphoethanolamine PEG derivatives such as 16:0 PEG5000PE, 14:0 PEG5000 PE, 18:0 PEG5000 PE, 18:1 PEG5000 PE, 16:0 PEG3000 PE, 14:0 PEG3000 PE, 18:0 PEG3000 PE, 18:1 PEG3000 PE, 16:0 PEG2000 PE, 14:0 PEG2000 PE, 18:0 PEG2000 PE, 18:1 PEG2000 PE 16:0 PEG1000 PE, 14:0 PEG1000 PE, 18:0 PEG1000 PE, 18:1 PEG 1000 PE, 16:0 PEG750 PE, 14:0 PEG750 PE, 18:0 PEG750 PE, 18:1 PEG750 PE, 16:0 PEG550 PE, 14:0 PEG550 PE, 18:0 PEG550 PE, 18:1 PEG550 PE, 16:0 PEG350 PE, 14:0 PEG350 PE, 18:0 PEG350 PE, and 18:1 PEG350, sterol PEG derivatives such as Chol-PEG600, and glycerol PEG derivatives such as DMG-PEG5000, DSG-PEG5000, DPG-PEG5000, DMG-PEG3000, DSG-PEG3000, DPG-PEG3000, DMG-PEG2000, DSG-PEG2000, DPG-PEG2000, DMG-PEG1000, DSG-PEG1000, DPG-PEG1000, DMG-PEG750, DSG-PEG750, DPG-PEG750, DMG-PEG550, DSG-PEG550, DPG-PEG550, DMG-PEG350, DSG-PEG350, and DPG-PEG350. In some embodiments, the lipid conjugate is a DMG-PEG. In some particular embodiments, the lipid conjugate is DMG-PEG2000. In some particular embodiments, the lipid conjugate is DMG-PEG5000.

It is within the level of a skilled artisan to select the cationic lipids, non-cationic lipids and/or lipid conjugates which comprise the lipid nanoparticle, as well as the relative molar ratio of such lipids to each other, such as based upon the characteristics of the selected lipid(s), the nature of the delivery to the intended target cells, and the characteristics of the nucleic acids and/or proteins to be delivered. Additional considerations include, for example, the saturation of the alkyl chain, as well as the size, charge, pH, pKa, fusogenicity and toxicity of the selected lipid(s). Thus, the molar ratios of each individual component may be adjusted accordingly.

The lipid nanoparticles for use in the method can be prepared by various techniques which are known to a skilled artisan. Nucleic acid-lipid particles and methods of preparation are disclosed in, for example, U.S. Patent Publication Nos. 20040142025 and 20070042031.

In some embodiments, the lipid nanoparticles will have a size within the range of about 25 to about 500 nm. In some embodiments, the lipid nanoparticles have a size from about 50 nm to about 300 nm, or from about 60 nm to about 120 nm. The size of the lipid nanoparticles may be determined by quasi-electric light scattering (QELS) as described in Bloomfield, Ann. Rev. Biophys. Bioeng., 10:421A150 (1981). A variety of methods are known in the art for producing a population of lipid nanoparticles of particular size ranges, for example, sonication or homogenization. One such method is described in U.S. Pat. No. 4,737,323.

In some embodiments, the lipid nanoparticles comprise a cell targeting molecule such as, for example, a targeting ligand (e.g., antibodies, scFv proteins, DART molecules, peptides, aptamers, and the like) anchored on the surface of the lipid nanoparticle that selectively binds the lipid nanoparticles to the targeted cell, such as any cell described herein.

In some embodiments, the vector exhibits tropism for one or more cell types. For example, the vector may exhibit liver cell and/or hepatocyte tropism, neural cell (e.g. neuron or glia) tropism, immune cell tropism, or tropism for any suitable cell type.

In some aspects, provided herein are pluralities of vectors that comprise any of the vectors described herein, and one or more additional vectors. In some embodiments, the one or more additional vectors comprise one or more additional polynucleotides encoding any additional transcriptional activation domain, multipartite effector such as multipartite activator, DNA-targeting domain, gRNA, fusion protein, DNA-targeting system, or a portion, component, or combination thereof. In some aspects, provided are pluralities of vectors, that include: a first vector comprising any of the polynucleotides described herein; a second vector comprising any of the polynucleotides described herein; and optionally one or more additional vectors comprising any of the polynucleotides described herein.

In some aspects, vectors provided herein may be referred to as delivery vehicles. In some aspects, any of the DNA-targeting systems, components thereof, or polynucleotides disclosed herein can be packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

Methods of introducing a nucleic acid into a host cell are known in the art, and any known method can be used to introduce a nucleic acid (e.g., an expression construct) into a cell. Suitable methods include, include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. Direct delivery of the RNP complex, including the DNA-targeting domain complexed with the sgRNA, can eliminate the need for intracellular transcription and translation and can offer a robust platform for host cells with low transcriptional and translational activity. The RNP complexes can be introduced into the host cell by any of the methods known in the art.

Nucleic acids or RNPs of the disclosure can be incorporated into a host using virus-like particles (VLP). VLPs contain normal viral vector components, such as envelope and capsids, but lack the viral genome. For instance, nucleic acids expressing the Cas and sgRNA can be fused to the viral vector components such as gag and introduced into producer cells. The resulting virus-like particles containing the sgRNA-expressing vectors can infect the host cell for efficient editing.

Introduction of the complexes, polypeptides, and nucleic acids of the disclosure can occur by protein transduction domains (PTDs). PTDs, including the human immunodeficiency virus-1 TAT, herpes simplex virus-1 VP22, Drosophila Antennapedia Antp, and the poluarginines, are peptide sequences that can cross the cell membrane, enter a host cell, and deliver the complexes, polypeptides, and nucleic acids into the cell.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like, for example as described in WO 2017/193107, WO 2016/123578, WO 2014/152432, WO 2014/093661, WO 2014/093655, or WO 2021/226555.

Various methods for the introduction of polynucleotides are well known and may be used with the provided methods and compositions. Exemplary methods include those for transfer of polynucleotides encoding the DNA targeting systems provided herein, including via viral, e.g., retroviral or lentiviral, transduction, transposons, and electroporation.

### C. Pharmaceutical Compositions and Formulations

Also provided are compositions, such as pharmaceutical compositions and formulations for administration, that include any of the DNA-targeting systems described herein, any of the gRNAs described herein, any of the combinations described herein, any of the fusion proteins described herein, any of the polynucleotides described herein, any of the pluralities of polynucleotides described herein, any of the vectors described herein, any of the pluralities of vectors described herein, or a portion or a component of any of the foregoing. In some aspects, the pharmaceutical composition comprises one or more pharmaceutically acceptable carriers.

In some aspects, the pharmaceutical composition contains one or more DNA-targeting systems provided herein or a component thereof. In some aspects, the pharmaceutical composition comprises one or more vectors, e.g., viral vectors that contain polynucleotides that encode one or more components of the DNA-targeting systems provided herein. Such compositions can be used in accord with the provided methods, and/or with the provided articles of manufacture or compositions, such as in the prevention or treatment of diseases, conditions, and disorders, or in detection, diagnostic, and prognostic methods.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

The pharmaceutical composition in some embodiments contains components in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The composition can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the composition. In some embodiments, it is administered by multiple bolus administrations of the composition, for example, over a period of no more than 3 days, or by continuous infusion administration of the composition.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

### IV. Methods of Modulating and Methods of Treatment

Provided herein are methods of treatment, e.g., including administering any of the compositions, such as pharmaceutical compositions described herein. In some aspects, also provided are methods of administering any of the compositions described herein to a subject, such as a subject that has a disease or disorder. The compositions, such as pharmaceutical compositions, described herein are useful in a variety of therapeutic, diagnostic and prophylactic indications. For example, the compositions are useful in treating a variety of diseases and disorders in a subject. Such methods and uses include therapeutic methods and uses, for example, involving administration of the compositions, to a subject having a disease, condition, or disorder, such as a tumor or cancer. In some embodiments, the e compositions are administered in an effective amount to effect treatment of the disease or disorder. Uses include uses of the compositions in such methods and treatments, and in the preparation of a medicament in order to carry out such therapeutic methods. In some embodiments, the methods are carried out by administering the compositions, to the subject having or suspected of having the disease or condition. In some embodiments, the methods thereby treat the disease or condition or disorder in the subject. Also provided are therapeutic methods for administering the cells and compositions to subjects, e.g., patients.

Also provided herein are methods for modulating the expression of frataxin (FXN) in a cell, that involve: introducing any of the DNA-targeting systems described herein, any of the gRNAs described herein, any of the combinations described herein, any of the fusion proteins described herein, any of the polynucleotides described herein, any of the pluralities of polynucleotides described herein, any of the vectors described herein, any of the pluralities of vectors described herein, or a portion or a component of any of the foregoing, into the cell.

Also provided herein are methods of inducing a genetic disruption at a target site in an enhancer region of a frataxin (FXN) locus in a cell, wherein the target site is located within the genomic coordinates human genome assembly GRCh38 (hg38) chr9:69,027,282-69,028,497, that involve: contacting a cell with any of the DNA-targeting systems described herein, any of the gRNAs described herein, any of the combinations described herein, any of the fusion proteins described herein, any of the polynucleotides described herein, any of the plurality of polynucleotides described herein, any of the vectors described herein, any of the pluralities of vectors described herein, or a portion or a component of any of the foregoing.

In some embodiments, the cell is from a subject that has or is suspected of having Friedreich's ataxia (FA).

Also provided herein are methods for modulating the expression of frataxin (FXN) in a subject, the method comprising: administering any of the DNA-targeting systems described herein, any of the gRNAs described herein, any of the combinations of gRNAs described herein, any of the fusion proteins described herein, any of the polynucleotides described herein, any of the pluralities of polynucleotides described herein, any of the vectors described herein, any of the plurality of vectors described herein, or a portion or a component of any of the foregoing, to the subject.

In some embodiments, the subject has or is suspected of having Friedreich's ataxia (FA).

Also provided herein are methods of treating Friedreich's ataxia (FA), the method comprising: administering any of the DNA-targeting systems described herein, any of the gRNAs described herein, any of the combinations described herein, any of the fusion proteins described herein, any of the polynucleotides described herein, any of the pluralities of polynucleotides described herein, any of the vectors described herein, any of the pluralities of vectors described herein, or a portion or a component of any of the foregoing, to a subject that has or is suspected of having FA.

In some embodiments, a cell in the subject exhibits reduced expression of FXN compared to a cell from a normal subject. In some embodiments, a cell in the subject has a GAA trinucleotide repeat expansion in the FXN gene. In some embodiments, the cell is a heart cell, a skeletal muscle cell, a nervous system cell, or an induced pluripotent stem cell. In some embodiments, the introducing, contacting or administering is carried out in vivo or ex vivo. In some embodiments, following the introducing, contacting or administering, the expression of frataxin (FXN) is increased in the cell or the subject. In some embodiments, the expression of frataxin (FXN) is increased at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold. In some embodiments, the expression is increased by less than about 10-fold, 9-fold, 8-fold, 7-fold or 6-fold. In some embodiments, the subject is a human.

Also provided herein is a pharmaceutical composition comprising any of the DNA-targeting systems described herein, any of the gRNAs described herein, any of the combinations described herein, any of the fusion proteins described herein, any of the polynucleotides described herein, any of the pluralities of polynucleotides described herein, any of the vectors described herein, any of the pluralities of vectors described herein, or a portion or a component of any of the foregoing.

In some of any of the provided embodiments, the pharmaceutical composition is for use in treating Friedreich's ataxia (FA). In some of any of the provided embodiments, the pharmaceutical composition is for use in the manufacture of a medicament for treating Friedreich's ataxia (FA).

In some of any of the provided embodiments, the pharmaceutical composition is to be administered to a subject.

Also provided herein is the use of any of the pharmaceutical compositions described herein for treating Friedreich's ataxia (FA).

Also provided herein is the use of any of the pharmaceutical compositions described herein in the manufacture of a medicament for treating Friedreich's ataxia (FA).

In some of any embodiments, the pharmaceutical composition is to be administered to a subject.

In some embodiments, the subject has or is suspected of having Friedreich's ataxia (FA). In some embodiments, a cell in the subject exhibits reduced expression of FXN compared to a cell from a normal subject. In some embodiments, a cell in the subject has a GAA trinucleotide repeat expansion in the FXN gene.

In some embodiments, the cell is a heart cell, a skeletal muscle cell, a nervous system cell, or an induced pluripotent stem cell. In some embodiments, the administration is carried out in vivo or ex vivo.

In some embodiments, following the administration, the expression of frataxin (FXN) is increased in the cell or the subject. In some embodiments, the expression is increased at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold. In some embodiments, the expression is increased by less than about 10-fold, 9-fold, 8-fold, 7-fold or 6-fold. In some embodiments, the subject is a human.

Also provided herein are cells comprising any of the DNA-targeting systems described herein, any of the gRNAs described herein, any of the combinations described herein, any of the fusion proteins described herein, any of the polynucleotides described herein, any of the pluralities of polynucleotides described herein, any of the vectors described herein, any of the pluralities of vectors described herein, or a portion or a component of any of the foregoing.

In some embodiments, the cell is a heart cell, a skeletal muscle cell, a nervous system cell, or an induced pluripotent stem cell. In some embodiments, the cell is from a subject that has or is suspected of having Friedreich's ataxia (FA).

In some aspects, methods of treating of treating a disease or disorder, such as diseases or disorders associated with dysregulation or reduced activity, function or expression of FXN, such as Friedreich's ataxia (FA), in an individual or a subject, involve administering to the individual or the subject AAV particles. The AAV particles may be administered to a particular tissue of interest, or it may be administered systemically. In some aspects, an effective amount of the AAV particles may be administered parenterally. Parenteral routes of administration may include without limitation intravenous, intraosseous, intra-arterial, intracerebral, intramuscular, intrathecal, subcutaneous, intracerebroventricular, and others. In some aspects, an effective amount of AAV particles may be administered through one route of administration. In some aspects, an effective amount of AAV particles may be administered through a combination of more than one route of administration. In some aspects, the individual is a mammal. In some aspects, the individual is a human.

An effective amount of AAV particles comprising an oversized AAV genome is administered, depending on the objectives of treatment. For example, where a low percentage of transduction can achieve the desired therapeutic effect, then the objective of treatment is generally to meet or exceed this level of transduction. In some instances, this level of transduction can be achieved by transduction of only about 1 to 5% of the target cells of the desired tissue type, In some aspects at least about 20% of the cells of the desired tissue type, In some aspects at least about 50%, In some aspects at least about 80%, In some aspects at least about 95%, In some aspects at least about 99% of the cells of the desired tissue type. As a guide, the number of particles administered per injection is generally between about 1×10⁶ and about 1×10¹⁴ particles, between about 1×10⁷ and 1×10¹³ particles, between about 1×10⁹ and 1×10¹² particles or about 1×10⁹ particles, about 1×10¹⁰ particles, or about 1×10¹¹ particles. The rAAV composition may be administered by one or more administrations, either during the same procedure or spaced apart by days, weeks, months, or years. One or more of any of the routes of administration described herein may be used. In some aspects, multiple vectors may be used to treat the human.

Methods to identify cells transduced by AAV viral particles can be employed; for example, immunohistochemistry or the use of a marker such as enhanced green fluorescent protein can be used to detect transduction of viral particles; for example viral particles comprising a rAAV capsid with one or more substitutions of amino acids.

In some aspects the AAV viral particles comprising an oversized AAV genome with are administered to more than one location simultaneously or sequentially. In some aspects, multiple injections of rAAV viral particles are no more than one hour, two hours, three hours, four hours, five hours, six hours, nine hours, twelve hours or 24 hours apart.

### V. Kits and Articles of Manufacture

Also provided are articles of manufacture, systems, apparatuses, and kits useful in performing the provided embodiments. In some embodiments, the provided articles of manufacture or kits contain one or more components of the one or more components of the DNA-targeting system provided herein. In some embodiments, the articles of manufacture or kits include polypeptides, nucleic acids, vectors and/or polynucleotides useful in performing the provided methods.

In some embodiments, the articles of manufacture or kits include one or more containers, typically a plurality of containers, packaging material, and a label or package insert on or associated with the container or containers and/or packaging, generally including instructions for use, e.g., instructions for introducing or administering.

Also provided are articles of manufacture, systems, apparatuses, and kits useful in administering the provided compositions, e.g., pharmaceutical compositions, e.g., for use in therapy or treatment. In some embodiments, the articles of manufacture or kits provided herein contain vectors and/or plurality of vectors, such as any vectors and/or plurality of vectors described herein. In some aspects, the articles of manufacture or kits provided herein can be used for administration of the vectors and/or plurality of vectors, and can include instructions for use.

The articles of manufacture and/or kits containing cells or cell compositions for therapy, may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition. The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VI. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”. In some embodiments, “about” may refer to ±25%, ±20%, ±15%, ±10%, ±5%, or ±1%.

In some aspects, corresponding positions of the one or more modifications, such as one or more substitutions, can be determined in reference to positions of a reference amino acid sequence or a reference nucleotide sequence. As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm or other available algorithms. By aligning the sequences, corresponding residues can be identified, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073). Alignment for determining corresponding positions can be obtained in various ways, for instance, using publicly available computer software such as BLAST, BLAST-2, ALIGN or Megalign (DNASTAR) software. Appropriate parameters for aligning sequences can be determined, including any algorithms needed to achieve maximal alignment over the full length of the sequences being compared. For example, corresponding residues can be determined by alignment of a reference sequence that is a wild-type Cas protein by available alignment methods. By aligning the sequences, one skilled in the art can identify corresponding residues, for example, using conserved and/or identical amino acid residues as guides.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as adenoviral vectors.

As used herein, “percent (%) amino acid sequence identity” and “percent identity” when used with respect to an amino acid sequence (reference polypeptide sequence) is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various known ways, in some embodiments, using publicly available computer software such as BLAST, BLAST-2, ALIGN or Megalign (DNASTAR) software. Appropriate parameters for aligning sequences can be determined, including any algorithms needed to achieve maximal alignment over the full length of the sequences being compared.

In some embodiments, “operably linked” may include the association of components, such as a DNA sequence, e.g. a heterologous nucleic acid) and a regulatory sequence(s), in such a way as to permit gene expression when the appropriate molecules (e.g. transcriptional activator proteins) are bound to the regulatory sequence. Hence, it means that the components described are in a relationship permitting them to function in their intended manner.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compounds, including cells. It may be a solution, a suspension, liquid, powder, a paste, aqueous, non-aqueous or any combination thereof.

As used herein, a “subject” is a mammal, such as a human or other animal, and typically is human.

### VII. Exemplary Embodiments

Among the provided embodiments are:

1. A DNA-targeting system comprising a DNA-targeting domain that binds to a target site in a regulatory DNA element of a frataxin (FXN) locus.

2. The DNA-targeting system of embodiment 1, wherein binding of the DNA-targeting domain to the target site does not introduce a genetic disruption or a DNA break at or near the target site.

3. The DNA-targeting system of embodiment 1 or 2, wherein the DNA-targeting domain comprises a Clustered Regularly Interspaced Short Palindromic Repeats associated (Cas)-guide RNA (gRNA) combination comprising (a) a Cas protein or a variant thereof and (b) at least one gRNA; a zinc finger protein (ZFP); a transcription activator-like effector (TALE); a meganuclease; a homing endonuclease; or an I-SceI enzyme or a variant thereof, optionally wherein the DNA-targeting domain comprises a catalytically inactive variant of any of the foregoing.

4. The DNA-targeting system of any of embodiments 1-3, wherein the DNA-targeting domain comprises a Cas-gRNA combination comprising (a) a Cas protein or a variant thereof and (b) at least one gRNA.

5. The DNA-targeting system of embodiment 3 or 4, wherein the variant Cas protein lacks nuclease activity or is a deactivated Cas (dCas) protein.

6. A DNA-targeting system comprising a DNA-targeting domain that is a Cas-guide RNA (gRNA) combination comprising:
(a) a variant Cas protein that lacks nuclease activity or that is a deactivated Cas (dCas) protein; and
(b) at least one gRNA, comprising a gRNA spacer sequence that is capable of hybridizing to a target site in a regulatory DNA element of a frataxin (FXN) locus or is complementary to the target site.

7. The DNA-targeting system of any of embodiments 3-6, wherein at least one gRNA is capable of complexing with the Cas protein or variant thereof.

8. The DNA-targeting system of any of embodiments 3-5 and 7, wherein at least one gRNA comprises a gRNA spacer sequence that is capable of hybridizing to the target site or is complementary to the target site.

9. The DNA-targeting system of any of embodiments 3-8, wherein the Cas protein or a variant thereof is a Cas9 protein or a variant thereof.

10. The DNA-targeting system of any of embodiments 4-9, wherein the variant Cas protein is a variant Cas9 protein that lacks nuclease activity or that is a deactivated Cas9 (dCas9) protein.

11. The DNA-targeting system of embodiment 9 or 10, wherein the Cas9 protein or a variant thereof is a Staphylococcus aureus Cas9 (SaCas9) protein or a variant thereof.

12. The DNA-targeting system of any of embodiments 9-11, wherein the variant Cas9 is a Staphylococcus aureus dCas9 protein (dSaCas9) that comprises at least one amino acid mutation selected from D10A and N580A, with reference to numbering of positions of SEQ ID NO:73.

13. The DNA-targeting system of any of embodiments 9-12, wherein the variant Cas9 protein comprises the sequence set forth in SEQ ID NO:72, or an amino acid sequence that has at least 90%, 91%, 92%, 93%, 94%, 95%, 96%, 97%, 98%, or 99% sequence identity thereto.

14. The DNA-targeting system of embodiment 9 or 10, wherein the Cas9 protein or variant thereof is a Streptococcus pyogenes Cas9 (SpCas9) protein or a variant thereof.

15. The DNA-targeting system of any of embodiments 9, 10, and 14, wherein the variant Cas9 is a Streptococcus pyogenes dCas9 (dSpCas9) protein that comprises at least one amino acid mutation selected from D10A and H840A, with reference to numbering of positions of SEQ ID NO:79.

16. The DNA-targeting system of any of embodiments 9, 10, 14, and 15, wherein the variant Cas9 protein comprises the sequence set forth in SEQ ID NO:78, or an amino acid sequence that has at least 90%, 91%, 92%, 93%, 94%, 95%, 96%, 97%, 98%, or 99% sequence identity thereto.

17. The DNA-targeting system of any of embodiments 1-16, wherein the regulatory DNA element is an enhancer.

18. The DNA-targeting system of embodiment 17, wherein the target site is located within the genomic coordinates human genome assembly GRCh38 (hg38) chr9:69,027,282-69,028,497.

19. The DNA-targeting system of embodiment 17 or 18, wherein the target site is located within the genomic coordinates hg38 chr9:69,027,615-69,028,101.

20. The DNA-targeting system of any of embodiments 17-19, wherein the target site comprises the sequence set forth in SEQ ID NO:21, a contiguous portion thereof of at least 14 nt, or a complementary sequence of any of the foregoing.

21. The DNA-targeting system of any of embodiments 3-20, wherein the at least one gRNA comprises a gRNA spacer sequence comprising the sequence set forth in SEQ ID NO:42, or a contiguous portion thereof of at least 14 nt.

22. The DNA-targeting system of embodiment 21, wherein the at least one gRNA further comprises the sequence set forth in SEQ ID NO:44.

23. The DNA-targeting system of any of embodiments 3-22, wherein the at least one gRNA comprises a gRNA that comprises the sequence set forth in SEQ ID NO:67, optionally wherein the at least one gRNA is the gRNA sequence set forth in SEQ ID NO:67.

24. The DNA-targeting system of any of embodiments 1-16, wherein the regulatory DNA element is a promoter.

25. The DNA-targeting system of embodiment 24, wherein the target site is located within the genomic coordinates hg38 chr9:68,940, 179-69,205,519.

26. The DNA-targeting system of embodiment 24 or 25, wherein the target site comprises a sequence selected from any one of SEQ ID NOS: 1-10, a contiguous portion thereof of at least 14 nt, or a complementary sequence of any of the foregoing.

27. The DNA-targeting system of any of embodiments 3-16 and 24-26, wherein the at least one gRNA comprises a gRNA spacer sequence comprising a sequence selected from any one of SEQ ID NOS:22-31, or a contiguous portion thereof of at least 14 nt.

28. The DNA-targeting system of embodiment 27, wherein the gRNA further comprises the sequence set forth in SEQ ID NO:44.

29. The DNA-targeting system of any of embodiments 3-16 and 24-28, wherein the at least one gRNA comprises a gRNA that comprises a sequence selected from any one of SEQ ID NOS:47-56, optionally wherein the at least one gRNA is the gRNA sequence set forth in any one of SEQ ID NOS:47-56.

30. The DNA-targeting system of embodiment 24 or 25, wherein the target site comprises a sequence selected from any one of SEQ ID NOS: 11-20, a contiguous portion thereof of at least 14 nt, or a complementary sequence of any of the foregoing.

31. The DNA-targeting system of any of embodiments 3-16, 24, 25 and 30, wherein the at least one gRNA comprises a gRNA spacer sequence comprising a sequence selected from any one of SEQ ID NOS:32-41, or a contiguous portion thereof of at least 14 nt.

32. The DNA-targeting system of embodiment 31, wherein the gRNA further comprises the sequence set forth in SEQ ID NO:46.

33. The DNA-targeting system of any of embodiments 3-16, 24, 25, and 30-32, wherein the at least one gRNA comprises a gRNA that comprises a sequence selected from any one of SEQ ID NOS:57-66, optionally wherein the at least one gRNA is the gRNA sequence set forth in any one of SEQ ID NOS:57-66.

34. The DNA-targeting system of any of embodiments 6-33, wherein the gRNA spacer sequence is between 14 nt and 24 nt, or between 16 nt and 22 nt in length.

35. The DNA-targeting system of any of embodiments 6-36, wherein the gRNA spacer sequence is 18 nt, 19 nt, 20 nt, 21 nt or 22 nt in length.

36. The DNA-targeting system of any of embodiments 3-35, wherein the gRNA comprises modified nucleotides for increased stability.

37. The DNA-targeting system of any of embodiments 1-36, wherein the DNA-targeting system further comprises at least one effector domain.

38. The DNA-targeting system of embodiment 37, wherein the DNA-targeting domain or a component thereof is fused to the at least one effector domain.

39. The DNA-targeting system of embodiment 38, wherein the DNA-targeting domain comprises a Cas-gRNA combination comprising (a) a Cas protein or a variant thereof and (b) at least one gRNA, and the component thereof fused to the at least one effector domain is the Cas protein or a variant thereof.

40. The DNA-targeting system of any of embodiments 37-39, wherein the effector domain induces, catalyzes or leads to transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation.

41. The DNA-targeting system of any of embodiments 37-40, wherein the effector domain induces, catalyzes or leads to transcription activation, transcription co-activation, transcription elongation.

42. The DNA-targeting system of any of embodiments 37-41, wherein the effector domain induces transcription activation.

43. A DNA-targeting system comprising a DNA-targeting domain that is a Cas-guide RNA (gRNA) combination comprising:
(a) a Staphylococcus aureus deactivated Cas9 protein (dSaCas9) protein set forth in SEQ ID NO:72 fused to at least one effector domain that induces transcription activation; and
(b) at least one gRNA that is a gRNA comprising a gRNA spacer sequence set forth in SEQ ID NO:42.

44. A DNA-targeting system comprising a DNA-targeting domain that is a Cas-guide RNA (gRNA) combination comprising:
(a) a Staphylococcus aureus deactivated Cas9 protein (dSaCas9) protein set forth in SEQ ID NO:72 fused to at least one effector domain that induces transcription activation; and
(b) at least one gRNA that is a gRNA comprising a gRNA spacer sequence set forth in SEQ ID NO:22.

45. A DNA-targeting system comprising a DNA-targeting domain that is a Cas-guide RNA (gRNA) combination comprising:
(a) a Staphylococcus aureus deactivated Cas9 protein (dSaCas9) protein set forth in SEQ ID NO:72 fused to at least one effector domain that induces transcription activation; and
(b) at least one gRNA that is a gRNA comprising a gRNA spacer sequence set forth in SEQ ID NO:28.

46. The DNA-targeting system of any of embodiments 37-45, wherein the effector domain comprises at least one VP16 domain, or a VP16 tetramer (“VP64”) or a variant thereof.

47. The DNA-targeting system of embodiment 46, wherein the effector domain comprises the sequence set forth in SEQ ID NO:81 or 83, or a portion thereof, or an amino acid sequence that has at least 90%, 91%, 92%, 93%, 94%, 95%, 96%, 97%, 98%, or 99% sequence identity to any of the foregoing.

48. The DNA-targeting system of any of embodiments 37-40, wherein the effector domain is selected from a p65 activation domain, a p300 domain, DPOLA, ENL, FOXO3, HSH2D, NCOA2, NCOA3, PSA1, PYGO1, RBM39, HERC2, DMD, or NOTCH2, or a domain thereof, a portion thereof or a variant thereof.

49. The DNA-targeting system of embodiment 48, wherein the effector domain comprises a sequence selected from any one of SEQ ID NOS: 113-125, or a domain thereof, a portion thereof, or an amino acid sequence that has at least 90%, 91%, 92%, 93%, 94%, 95%, 96%, 97%, 98%, or 99% sequence identity to any of the foregoing.

50. The DNA-targeting system of embodiment 48 or 49, wherein the effector domain comprises a sequence selected from any one of SEQ ID NOS: 100-112, or a domain thereof, a portion thereof, or an amino acid sequence that has at least 90%, 91%, 92%, 93%, 94%, 95%, 96%, 97%, 98%, or 99% sequence identity to any of the foregoing.

51. The DNA-targeting system of any of embodiments 46-50, wherein the variant thereof comprises a truncation thereof.

52. The DNA-targeting system of any of embodiments 37-51, wherein the at least one effector domain is fused to the N-terminus, the C-terminus, or both the N-terminus and the C-terminus, of the DNA-targeting domain or a component thereof.

53. The DNA-targeting system of any of embodiments 37-52, further comprising one or more linkers connecting the DNA-targeting domain or a component thereof to the at least one effector domain, and/or further comprising one or more nuclear localization signals (NLS).

54. The DNA-targeting system of any of embodiments 37-47, 52 and 53, wherein the DNA-targeting system comprises the sequence set forth in SEQ ID NO:71, or an amino acid sequence that has at least 90%, 91%, 92%, 93%, 94%, 95%, 96%, 97%, 98%, or 99% sequence identity thereto.

55. The DNA-targeting system of any of embodiments 37-47, 52 and 53, wherein the DNA-targeting system comprises the sequence set forth in SEQ ID NO:77, or an amino acid sequence that has at least 90%, 91%, 92%, 93%, 94%, 95%, 96%, 97%, 98%, or 99% sequence identity thereto.

56. The DNA-targeting system of any of embodiments 1-55, wherein the DNA-targeting domain is a first DNA-targeting domain, and the DNA-targeting system further comprises one or more second DNA-targeting domains.

57. The DNA-targeting system of embodiment 56, wherein:
the first DNA-targeting domain binds a first target site in an enhancer of a FXN locus; and
the second DNA-targeting domain binds a second target site in a promoter of a FXN locus.

58. A DNA-targeting system that binds to one or more target sites in a regulatory DNA element of a frataxin (FXN) locus, the DNA-targeting system comprising:
a first DNA-targeting domain that binds a first target site in an enhancer of a FXN locus; and
a second DNA-targeting domain that binds a second target site in a promoter of a FXN locus.

59. The DNA-targeting system of embodiment 57 or 58, wherein:
the first target site is located within the genomic coordinates human genome assembly GRCh38 (hg38) chr9:69,027,282-69,028,497; and
the second target site is located within the genomic coordinates hg38 chr9:68,940,179-69,205,519.

60. The DNA-targeting system of any of embodiments 57-59, wherein:
the first target site is located within the genomic coordinates hg38 chr9:69,027,615-69,028,101; and
the second target site is located within the genomic coordinates hg38 chr9:68,940,179-69,205,519.

61. The DNA-targeting system of any of embodiments 56-60, wherein:
the first DNA-targeting domain comprises a first Cas-gRNA combination comprising (a) a first Cas protein or a variant thereof and (b) a first gRNA that is capable of hybridizing to the target site or is complementary to the first target site; and
the second DNA-targeting domain comprises a second Cas-gRNA combination comprising (a) a second Cas protein or a variant thereof and (b) a second gRNA that is capable of hybridizing to the target site or is complementary to the second target site.

62. The DNA-targeting system of any of embodiments 56-61, wherein:
the first DNA-targeting domain comprises a first Cas-gRNA combination comprising (a) a first Cas protein or a variant thereof and (b) a first gRNA comprising a gRNA spacer sequence set forth in SEQ ID NO:42 or a contiguous portion thereof of at least 14 nt.

63. The DNA-targeting system of any of embodiments 56-62, wherein:
the second DNA-targeting domain comprises a second Cas-gRNA combination comprising (a) a second Cas protein or a variant thereof and (b) a second gRNA comprising a gRNA spacer sequence set forth in SEQ ID NO:22 or 28 or a contiguous portion thereof of at least 14 nt.

64. The DNA-targeting system of embodiment 62 or 63, wherein:
the first Cas-gRNA combination comprises (a) a first Cas protein or a variant thereof and (b) a first gRNA comprising a gRNA spacer sequence set forth in SEQ ID NO:42 or a contiguous portion thereof of at least 14 nt; and
the second Cas-gRNA combination comprises (a) a second Cas protein or a variant thereof and (b) a second gRNA comprising a gRNA spacer sequence set forth in SEQ ID NO:22 or a contiguous portion thereof of at least 14 nt.

65. The DNA-targeting system of embodiment 62 or 63, wherein:
the first Cas-gRNA combination comprises (a) a first Cas protein or a variant thereof and (b) a first gRNA comprising a gRNA spacer sequence set forth in SEQ ID NO:42 or a contiguous portion thereof of at least 14 nt; and
the second Cas-gRNA combination comprises (a) a second Cas protein or a variant thereof and (b) a second gRNA comprising a gRNA spacer sequence set forth in SEQ ID NO:28 or a contiguous portion thereof of at least 14 nt.

66. The DNA-targeting system of embodiment 56, wherein:
the first DNA-targeting domain binds a first target site in a promoter of a FXN locus; and
the second DNA-targeting domain binds a second target site in a promoter of a FXN locus.

67. A DNA-targeting system that binds to one or more target sites in a regulatory DNA element of a frataxin (FXN) locus, the DNA-targeting system comprising:
a first DNA-targeting domain that binds a first target site in a promoter of a FXN locus; and
a second DNA-targeting domain that binds a second target site in a promoter of a FXN locus.

68. The DNA-targeting system of embodiment 66 or 67, wherein the first target site and the second target site independently are located within the genomic coordinates hg38 chr9:68,940,179-69,205,519.

69. The DNA-targeting system of any of embodiments 66-68, wherein the first target site and the second target site are different.

70. The DNA-targeting system of any of embodiments 56 and 66-69, wherein:
the first DNA-targeting domain comprises a first Cas-gRNA combination comprising (a) a first Cas protein or a variant thereof and (b) a first gRNA that is capable of hybridizing to the target site or is complementary to the first target site; and
the second DNA-targeting domain comprises a second Cas-gRNA combination comprising (a) a second Cas protein or a variant thereof and (b) a second gRNA that is capable of hybridizing to the target site or is complementary to the second target site.

71. The DNA-targeting system of any of embodiments 56 and 66-70, wherein:
the first DNA-targeting domain comprises a first Cas-gRNA combination comprising (a) a first Cas protein or a variant thereof and (b) a first gRNA comprising a gRNA spacer sequence set forth in SEQ ID NO:22 or a contiguous portion thereof of at least 14 nt.

72. The DNA-targeting system of any of embodiments 56 and 66-71, wherein:
the second DNA-targeting domain comprises a second Cas-gRNA combination comprising (a) a second Cas protein or a variant thereof and (b) a second gRNA comprising a gRNA spacer sequence set forth in SEQ ID NO:28 or a contiguous portion thereof of at least 14 nt.

73. The DNA-targeting system of embodiment 71 or 72, wherein:
the first Cas-gRNA combination comprises (a) a first Cas protein or a variant thereof and (b) a first gRNA comprising a gRNA spacer sequence set forth in SEQ ID NO:22 or a contiguous portion thereof of at least 14 nt; and
the second Cas-gRNA combination comprises (a) a second Cas protein or a variant thereof and (b) a second gRNA comprising a gRNA spacer sequence set forth in SEQ ID NO:28 or a contiguous portion thereof of at least 14 nt.

74. The DNA-targeting system of any of embodiments 61-65 and 70-73, wherein the first Cas protein or a variant thereof and/or the second Cas protein or a variant thereof is a variant Cas9 protein that lacks nuclease activity or that is a deactivated Cas9 (dCas9) protein.

75. The DNA-targeting system of embodiment 74, wherein the first variant Cas protein and/or the second variant Cas protein is a Staphylococcus aureus dCas9 protein (dSaCas9) that comprises at least one amino acid mutation selected from D10A and N580A, with reference to numbering of positions of SEQ ID NO:73; or comprises the sequence set forth in SEQ ID NO:72, or an amino acid sequence that has at least 90%, 91%, 92%, 93%, 94%, 95%, 96%, 97%, 98%, or 99% sequence identity thereto.

76. The DNA-targeting system of embodiment 74, wherein the first variant Cas protein and/or the second variant Cas protein is a Streptococcus pyogenes dCas9 (dSpCas9) protein that comprises at least one amino acid mutation selected from D10A and H840A, with reference to numbering of positions of SEQ ID NO:79; or comprises the sequence set forth in SEQ ID NO:78, or an amino acid sequence that has at least 90%, 91%, 92%, 93%, 94%, 95%, 96%, 97%, 98%, or 99% sequence identity thereto.

77. The DNA-targeting system of any of embodiments 61-65 and 70-76, wherein the first Cas protein and the second Cas protein are the same.

78. The DNA-targeting system of any of embodiments 61-65 and 70-76, wherein the first Cas protein and the second Cas protein are different.

79. The DNA-targeting system of any of embodiments 61-65 and 70-78, wherein the first Cas protein or a variant thereof and/or the second Cas protein or a variant thereof is fused to at least one effector domain.

80. The DNA-targeting system of embodiment 79, wherein the effector domain induces, catalyzes or leads to transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation.

81. The DNA-targeting system of embodiment 79 or 80, wherein the effector domain induces transcription activation.

82. The DNA-targeting system of any of embodiments 56-81, wherein the first DNA-targeting domain and the second DNA-targeting domain are encoded in a first polynucleotide.

83. The DNA-targeting system of any of embodiments 56-82, wherein the first Cas protein and the second Cas protein are encoded in a first polynucleotide.

84. The DNA-targeting system of any of embodiments 56-77 and 79-83, wherein the first Cas protein and the second Cas protein are encoded by the same nucleotide sequence.

85. The DNA-targeting system of any of embodiments 56-84, wherein the first gRNA and the second gRNA are encoded in a first polynucleotide.

86. The DNA-targeting system of any of embodiments 56-77 and 79-85, wherein the first Cas protein and the second Cas protein are encoded by the same nucleotide sequence, and the Cas protein, the first gRNA, and the second gRNA are encoded in a first polynucleotide.

87. The DNA-targeting system of any of embodiments 56-81, wherein the first DNA-targeting domain is encoded in a first polynucleotide and the second DNA-targeting domain is encoded in a second polynucleotide.

88. The DNA-targeting system of any of embodiments 56-81 and 87, wherein the first Cas protein is encoded in a first polynucleotide and the second Cas protein is encoded in a second polynucleotide.

89. The DNA-targeting system of any of embodiments 56-81, 87, and 88, wherein the first gRNA is encoded in a first polynucleotide and the second gRNA is encoded in a second polynucleotide.

90. The DNA-targeting system of any of embodiments 56-81 and 87-89, wherein the first Cas protein and the first gRNA are encoded in a first polynucleotide, and the second Cas protein and the second gRNA are encoded in a second polynucleotide.

56. A combination, comprising:
a first DNA-targeting domain comprising the DNA targeting domain of any one of embodiments 1-55, and
one or more second DNA-targeting domains, optionally wherein the one or more second DNA-targeting domains comprises the DNA targeting domain of any one of embodiments 1-55.

57. The combination of embodiment 56, wherein:
the first DNA-targeting domain binds a first target site in an enhancer of a FXN locus; and
the second DNA-targeting domain binds a second target site in a promoter of a FXN locus.

58. A combination comprising:
a first DNA-targeting domain that binds a first target site in an enhancer of a frataxin (FXN) locus; and
a second DNA-targeting domain that binds a second target site in a promoter of a FXN locus.

59. The combination of embodiment 57 or 58, wherein:
the first target site is located within the genomic coordinates human genome assembly GRCh38 (hg38) chr9:69,027,282-69,028,497; and
the second target site is located within the genomic coordinates hg38 chr9:68,940,179-69,205,519.

60. The combination of any of embodiments 57-59, wherein:
the first target site is located within the genomic coordinates hg38 chr9:69,027,615-69,028,101; and
the second target site is located within the genomic coordinates hg38 chr9:68,940,179-69,205,519.

61. The combination of any of embodiments 56-60, wherein:
the first DNA-targeting domain comprises a first Cas-gRNA combination comprising (a) a first Cas protein or a variant thereof and (b) a first gRNA that is capable of hybridizing to the target site or is complementary to the first target site; and
the second DNA-targeting domain comprises a second Cas-gRNA combination comprising (a) a second Cas protein or a variant thereof and (b) a second gRNA that is capable of hybridizing to the target site or is complementary to the second target site.

62. The combination of any of embodiments 56-61, wherein:
the first DNA-targeting domain comprises a first Cas-gRNA combination comprising (a) a first Cas protein or a variant thereof and (b) a first gRNA comprising a gRNA spacer sequence set forth in SEQ ID NO:42 or a contiguous portion thereof of at least 14 nt.

63. The combination of any of embodiments 56-62, wherein:
the second DNA-targeting domain comprises a second Cas-gRNA combination comprising (a) a second Cas protein or a variant thereof and (b) a second gRNA comprising a gRNA spacer sequence set forth in SEQ ID NO:22 or 28 or a contiguous portion thereof of at least 14 nt.

64. The combination of embodiment 62 or 63, wherein:
the first Cas-gRNA combination comprises (a) a first Cas protein or a variant thereof and (b) a first gRNA comprising a gRNA spacer sequence set forth in SEQ ID NO:42 or a contiguous portion thereof of at least 14 nt; and
the second Cas-gRNA combination comprises (a) a second Cas protein or a variant thereof and (b) a second gRNA comprising a gRNA spacer sequence set forth in SEQ ID NO:22 or a contiguous portion thereof of at least 14 nt.

65. The combination of embodiment 62 or 63, wherein:
the first Cas-gRNA combination comprises (a) a first Cas protein or a variant thereof and (b) a first gRNA comprising a gRNA spacer sequence set forth in SEQ ID NO:42 or a contiguous portion thereof of at least 14 nt; and
the second Cas-gRNA combination comprises (a) a second Cas protein or a variant thereof and (b) a second gRNA comprising a gRNA spacer sequence set forth in SEQ ID NO:28 or a contiguous portion thereof of at least 14 nt.

66. The combination of embodiment 56, wherein:
the first DNA-targeting domain binds a first target site in a promoter of a FXN locus; and
the second DNA-targeting domain binds a second target site in a promoter of a FXN locus.

67. A combination comprising:
a first DNA-targeting domain that binds a first target site in a promoter of a frataxin (FXN) locus; and
a second DNA-targeting domain that binds a second target site in a promoter of a FXN locus.

68. The combination of embodiment 66 or 67, wherein the first target site and the second target site independently are located within the genomic coordinates hg38 chr9:68,940,179-69,205,519.

69. The combination of any of embodiments 66-68, wherein the first target site and the second target site are different.

70. The combination of any of embodiments 56 and 66-69, wherein:
the first DNA-targeting domain comprises a first Cas-gRNA combination comprising (a) a first Cas protein or a variant thereof and (b) a first gRNA that is capable of hybridizing to the target site or is complementary to the first target site; and
the second DNA-targeting domain comprises a second Cas-gRNA combination comprising (a) a second Cas protein or a variant thereof and (b) a second gRNA that is capable of hybridizing to the target site or is complementary to the second target site.

71. The combination of any of embodiments 56 and 66-70, wherein:
the first DNA-targeting domain comprises a first Cas-gRNA combination comprising (a) a first Cas protein or a variant thereof and (b) a first gRNA comprising a gRNA spacer sequence set forth in SEQ ID NO:22 or a contiguous portion thereof of at least 14 nt.

72. The combination of any of embodiments 56 and 66-71, wherein:
the second DNA-targeting domain comprises a second Cas-gRNA combination comprising (a) a second Cas protein or a variant thereof and (b) a second gRNA comprising a gRNA spacer sequence set forth in SEQ ID NO:28 or a contiguous portion thereof of at least 14 nt.

73. The combination of embodiment 71 or 72, wherein:
the first Cas-gRNA combination comprises (a) a first Cas protein or a variant thereof and (b) a first gRNA comprising a gRNA spacer sequence set forth in SEQ ID NO:22 or a contiguous portion thereof of at least 14 nt; and
the second Cas-gRNA combination comprises (a) a second Cas protein or a variant thereof and (b) a second gRNA comprising a gRNA spacer sequence set forth in SEQ ID NO:28 or a contiguous portion thereof of at least 14 nt.

74. The combination of any of embodiments 61-65 and 70-73, wherein the first Cas protein or a variant thereof and/or the second Cas protein or a variant thereof is a variant Cas9 protein that lacks nuclease activity or that is a deactivated Cas9 (dCas9) protein.

75. The combination of embodiment 74, wherein the first variant Cas protein and/or the second variant Cas protein is a Staphylococcus aureus dCas9 protein (dSaCas9) that comprises at least one amino acid mutation selected from D10A and N580A, with reference to numbering of positions of SEQ ID NO:73; or comprises the sequence set forth in SEQ ID NO:72, or an amino acid sequence that has at least 90%, 91%, 92%, 93%, 94%, 95%, 96%, 97%, 98%, or 99% sequence identity thereto.

76. The combination of embodiment 74, wherein the first variant Cas protein and/or the second variant Cas protein is a Streptococcus pyogenes dCas9 (dSpCas9) protein that comprises at least one amino acid mutation selected from D10A and H840A, with reference to numbering of positions of SEQ ID NO:79; or comprises the sequence set forth in SEQ ID NO:78, or an amino acid sequence that has at least 90%, 91%, 92%, 93%, 94%, 95%, 96%, 97%, 98%, or 99% sequence identity thereto.

77. The combination of any of embodiments 61-65 and 70-76, wherein the first Cas protein and the second Cas protein are the same.

78. The combination of any of embodiments 61-65 and 70-76, wherein the first Cas protein and the second Cas protein are different.

79. The combination of any of embodiments 61-65 and 70-78, wherein the first Cas protein or a variant thereof and/or the second Cas protein or a variant thereof is fused to at least one effector domain.

80. The combination of embodiment 79, wherein the effector domain induces, catalyzes or leads to transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation.

81. The combination of embodiment 79 or 80, wherein the effector domain induces transcription activation.

82. The combination of any of embodiments 56-81, wherein the first DNA-targeting domain and the second DNA-targeting domain are encoded in a first polynucleotide.

83. The combination of any of embodiments 56-82, wherein the first Cas protein and the second Cas protein are encoded in a first polynucleotide.

84. The combination of any of embodiments 56-77 and 79-83, wherein the first Cas protein and the second Cas protein are encoded by the same nucleotide sequence.

85. The combination of any of embodiments 56-84, wherein the first gRNA and the second gRNA are encoded in a first polynucleotide.

86. The combination of any of embodiments 56-77 and 79-85, wherein the first Cas protein and the second Cas protein are encoded by the same nucleotide sequence, and the Cas protein, the first gRNA, and the second gRNA are encoded in a first polynucleotide.

87. The combination of any of embodiments 56-81, wherein the first DNA-targeting domain is encoded in a first polynucleotide and the second DNA-targeting domain is encoded in a second polynucleotide.

88. The combination of any of embodiments 56-81 and 87, wherein the first Cas protein is encoded in a first polynucleotide and the second Cas protein is encoded in a second polynucleotide.

89. The combination of any of embodiments 56-81, 87, and 88, wherein the first gRNA is encoded in a first polynucleotide and the second gRNA is encoded in a second polynucleotide.

90. The combination of any of embodiments 56-81 and 87-89, wherein the first Cas protein and the first gRNA are encoded in a first polynucleotide, and the second Cas protein and the second gRNA are encoded in a second polynucleotide.

91. A guide RNA (gRNA) that binds a target site in an enhancer region of a frataxin (FXN) locus, wherein the target site is located within the genomic coordinates human genome assembly GRCh38 (hg38) chr9:69,027,282-69,028,497.

92. The gRNA of embodiment 91, wherein the target site is located within the genomic coordinates hg38 chr9:69,027,615-69,028,101.

93. The gRNA of any of embodiments 91 or 92, wherein the target site comprises the sequence set forth in SEQ ID NO:21, a contiguous portion thereof of at least 14 nt, or a complementary sequence of any of the foregoing.

94. The gRNA of any of embodiments 91-93, wherein the gRNA comprises a gRNA spacer sequence comprising the sequence set forth in SEQ ID NO:42, or a contiguous portion thereof of at least 14 nt.

95. The gRNA of any of embodiments 91-94, wherein the gRNA further comprises the sequence set forth in SEQ ID NO:44.

96. The gRNA of any of embodiments 91-95, wherein the gRNA comprises the sequence set forth in SEQ ID NO:67, optionally wherein the gRNA is set forth in SEQ ID NO:67.

97. A guide RNA (gRNA) that binds a target site in a regulatory DNA element of a frataxin (FXN) locus wherein the target site comprises a sequence selected from any one of SEQ ID NOS:1-10, a contiguous portion thereof of at least 14 nt, or a complementary sequence of any of the foregoing.

98. The gRNA of embodiment 97, wherein the gRNA comprises a gRNA spacer sequence comprising a sequence selected from any one of SEQ ID NOS:22-31, or a contiguous portion thereof of at least 14 nt.

99. The gRNA of embodiment 97 or 98, wherein the gRNA further comprises the sequence set forth in SEQ ID NO:44.

100. The gRNA of any of embodiments 97-99, wherein the gRNA comprises a sequence selected from any one of SEQ ID NOS:47-56, optionally wherein the gRNA is set forth in any one of SEQ ID NOS:47-56, optionally wherein the gRNA is set forth in SEQ ID NO:47 or 53.

101. A guide RNA (gRNA) that binds a target site in a regulatory DNA element of a frataxin (FXN) locus wherein the target site comprises a sequence selected from any one of SEQ ID NOS:11-20, a contiguous portion thereof of at least 14 nt, or a complementary sequence of any of the foregoing.

102. The gRNA of embodiment 101, wherein the gRNA comprises a gRNA spacer sequence comprising a sequence selected from any one of SEQ ID NOS:32-41, or a contiguous portion thereof of at least 14 nt.

103. The gRNA of embodiment 101 or 102, wherein the gRNA further comprises the sequence set forth in SEQ ID NO:46.

104. The gRNA of any of embodiments 101-103, wherein the gRNA comprises a sequence selected from any one of SEQ ID NOS:57-66, optionally wherein the gRNA is set forth in any one of SEQ ID NOS:57-66.

105. The gRNA of any of embodiments 91-104, wherein the gRNA spacer sequence is between 14 nt and 24 nt, or between 16 nt and 22 nt in length.

106. The gRNA of any of embodiments 91-105, wherein the gRNA spacer sequence is 18 nt, 19 nt, 20 nt, 21 nt or 22 nt in length.

107. The gRNA of any of embodiments 91-106, wherein the gRNA comprises modified nucleotides for increased stability.

108. The gRNA of any of embodiments 91-107, wherein the gRNA is capable of complexing with the Cas protein or variant thereof.

109. The gRNA of any of embodiments 91-108, wherein the gRNA comprises a gRNA spacer sequence that is capable of hybridizing to the target site or is complementary to the target site.

110. A combination, comprising a first gRNA comprising the gRNA of any of embodiments 91-109, and one or more second gRNAs that binds to a second target site in a regulatory DNA element of a frataxin (FXN) locus.

111. The combination of embodiment 110, wherein the second gRNA comprises the gRNA of any of embodiments 91-109.

112. A combination, comprising:
a first gRNA that binds a first target site in an enhancer region of a frataxin (FXN) locus, wherein the first target site is located within the genomic coordinates human genome assembly GRCh38 (hg38) chr9:69,027,282-69,028,497; and
a second gRNA that binds a second target site in a promoter region of a FXN locus, wherein the second target site is located within the genomic coordinates hg38 chr9:68,940, 179-69,205,519.

113. The combination of embodiment 112, wherein the first gRNA comprises a gRNA spacer sequence set forth in SEQ ID NO:42 or a contiguous portion thereof of at least 14 nt.

114. The combination of embodiment 112 or 113, wherein the second gRNA comprises a gRNA spacer sequence set forth in SEQ ID NO:22 or 28 or a contiguous portion thereof of at least 14 nt.

115. The combination of any of embodiments 112-114, wherein:
the first gRNA comprises a gRNA spacer sequence set forth in SEQ ID NO:42 or a contiguous portion thereof of at least 14 nt; and
the second gRNA comprises a gRNA spacer sequence set forth in SEQ ID NO:22 or a contiguous portion thereof of at least 14 nt.

116. The combination of any of embodiments 112-114, wherein:
the first gRNA comprises a gRNA spacer sequence set forth in SEQ ID NO:42 or a contiguous portion thereof of at least 14 nt; and
the second gRNA comprises a gRNA spacer sequence set forth in SEQ ID NO:28 or a contiguous portion thereof of at least 14 nt.

117. A combination, comprising:
a first gRNA that binds a first target site in a promoter region of a frataxin (FXN) locus, wherein the first target site is located within the genomic coordinates hg38 chr9:68,940,179-69,205,519; and
a second gRNA that binds a second target site in a promoter region of a FXN locus, wherein the second target site is located within the genomic coordinates hg38 chr9:68,940, 179-69,205,519.

118. The combination of embodiment 117, wherein the combination comprises:
the first gRNA comprises a gRNA spacer sequence set forth in SEQ ID NO:22 or a contiguous portion thereof of at least 14 nt; and
the second gRNA comprises a gRNA spacer sequence set forth in SEQ ID NO:28 or a contiguous portion thereof of at least 14 nt.

119. A fusion protein comprising (1) a DNA-targeting domain or a component thereof and (2) at least one effector domain, wherein:
the DNA-targeting domain or a component thereof binds to a target site in a regulatory DNA element of a frataxin (FXN) locus; and
the effector domain induces, catalyzes or leads to transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation.

120. The fusion protein of embodiment 119, wherein binding of the DNA-targeting domain or a component thereof to the target site does not introduce a genetic disruption or a DNA break at or near the target site.

121. The fusion protein of embodiment 119 or 120, wherein the DNA-targeting domain comprises a Clustered Regularly Interspaced Short Palindromic Repeats associated (Cas)-guide RNA (gRNA) combination comprising (a) a Cas protein or a variant thereof and (b) at least one gRNA; a zinc finger protein (ZFP); a transcription activator-like effector (TALE); a meganuclease; a homing endonuclease; or an I-SceI enzyme or a variant thereof, optionally wherein the DNA-targeting domain comprises a catalytically inactive variant of any of the foregoing.

122. The fusion protein of any of embodiments 190-121, wherein the DNA-targeting domain comprises a Cas-gRNA combination comprising a Cas protein or a variant thereof and at least one gRNA, and the component of the DNA-targeting domain is a Cas protein or a variant thereof.

123. A fusion protein comprising (1) a Cas protein or a variant thereof and (2) at least one effector domain, wherein the effector domain induces, catalyzes or leads to transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation.

124. The fusion protein of any of embodiments 121-123, wherein the variant Cas protein lacks nuclease activity or is a deactivated Cas (dCas) protein.

125. The fusion protein of any of embodiments 121-124, wherein the Cas protein or a variant thereof is a Cas9 protein or a variant thereof.

126. The fusion protein of any of embodiments 121-125, wherein the variant Cas protein is a variant Cas9 protein that lacks nuclease activity or that is a deactivated Cas9 (dCas9) protein.

127. The fusion protein of embodiment 125 or 126, wherein the Cas9 protein or a variant thereof is a Staphylococcus aureus Cas9 (SaCas9) protein or a variant thereof.

128. The fusion protein of any of embodiments 125-127, wherein the variant Cas9 is a Staphylococcus aureus dCas9 protein (dSaCas9) that comprises at least one amino acid mutation selected from D10A and N580A, with reference to numbering of positions of SEQ ID NO:73.

129. The fusion protein of any of embodiments 125-128, wherein the variant Cas9 protein comprises the sequence set forth in SEQ ID NO:72, or an amino acid sequence that has at least 90%, 91%, 92%, 93%, 94%, 95%, 96%, 97%, 98%, or 99% sequence identity thereto.

130. The fusion protein of embodiment 125 or 126, wherein the Cas9 protein or variant thereof is a Streptococcus pyogenes Cas9 (SpCas9) protein or a variant thereof.

131. The fusion protein of any of embodiments 125, 126, and 130, wherein the variant Cas9 is a Streptococcus pyogenes dCas9 (dSpCas9) protein that comprises at least one amino acid mutation selected from D10A and H840A, with reference to numbering of positions of SEQ ID NO:79.

132. The fusion protein of any of embodiments 125, 126, 130, and 131, wherein the variant Cas9 protein comprises the sequence set forth in SEQ ID NO:78, or an amino acid sequence that has at least 90%, 91%, 92%, 93%, 94%, 95%, 96%, 97%, 98%, or 99% sequence identity thereto.

133. The fusion protein of any of embodiments 119-132, wherein the regulatory DNA element is an enhancer.

134. The fusion protein of embodiment 133, wherein the target site is located within the genomic coordinates human genome assembly GRCh38 (hg38) chr9:69,027,282-69,028,497.

135. The fusion protein of embodiment 133 or 134, wherein the target site is located within the genomic coordinates hg38 chr9:69,027,615-69,028,101.

136. The fusion protein of any of embodiments 133-135, wherein the target site comprises the sequence set forth in SEQ ID NO:21, a contiguous portion thereof of at least 14 nt, or a complementary sequence of any of the foregoing.

137. The fusion protein of any of embodiments 119-122, wherein the regulatory DNA element is a promoter.

138. The fusion protein of embodiment 137, wherein the target site is located within the genomic coordinates hg38 chr9:68,940, 179-69,205,519.

139. The fusion protein of embodiment 137 or 138, wherein the target site comprises a sequence selected from any one of SEQ ID NOS: 1-10, a contiguous portion thereof of at least 14 nt, or a complementary sequence of any of the foregoing.

140. The fusion protein of embodiment 137 or 138, wherein the target site comprises a sequence selected from any one of SEQ ID NOS: 11-20, a contiguous portion thereof of at least 14 nt, or a complementary sequence of any of the foregoing.

141. The fusion protein of any of embodiments 119-140, wherein the effector domain induces, catalyzes or leads to transcription activation, transcription co-activation, transcription elongation.

142. The fusion protein of any of embodiments 119-141, wherein the effector domain induces transcription activation.

143. The fusion protein of any of embodiments 119-142, wherein the effector domain comprises at least one VP16 domain, or a VP16 tetramer (“VP64”) or a variant thereof.

144. The fusion protein of embodiment 143, wherein the effector domain comprises the sequence set forth in SEQ ID NO:81 or 83, or a portion thereof, or an amino acid sequence that has at least 90%, 91%, 92%, 93%, 94%, 95%, 96%, 97%, 98%, or 99% sequence identity to any of the foregoing. 145. The fusion protein of any of embodiments 119-140, wherein the effector domain is selected from a p65 activation domain, a p300 domain, DPOLA, ENL, FOXO3, HSH2D, NCOA2, NCOA3, PSA1, PYGO1, RBM39, HERC2, DMD, or NOTCH2, or a domain thereof, a portion thereof or a variant thereof.

146. The fusion protein of embodiment 145, wherein the effector domain comprises a sequence selected from any one of SEQ ID NOS: 113-125, or a domain thereof, a portion thereof, or an amino acid sequence that has at least 90%, 91%, 92%, 93%, 94%, 95%, 96%, 97%, 98%, or 99% sequence identity to any of the foregoing.

147. The fusion protein of embodiment 145 or 146, wherein the effector domain comprises a sequence selected from any one of SEQ ID NOS: 100-112, or a domain thereof, a portion thereof, or an amino acid sequence that has at least 90%, 91%, 92%, 93%, 94%, 95%, 96%, 97%, 98%, or 99% sequence identity to any of the foregoing.

148. The fusion protein of any of embodiments 143-147, wherein the variant thereof comprises a truncations thereof.

149. The fusion protein of any of embodiments 119-148, wherein the at least one effector domain is fused to the N-terminus, the C-terminus, or both the N-terminus and the C-terminus, of the DNA-targeting domain or a component thereof, optionally wherein the at least one effector domain is fused to the N-terminus, the C-terminus, or both the N-terminus and the C-terminus of the Cas protein or a variant thereof.

150. The fusion protein of any of embodiments 119-149, further comprising one or more linkers connecting the DNA-targeting domain or a component thereof, optionally the Cas protein or variant thereof, to the at least one effector domain, and/or further comprising one or more nuclear localization signals (NLS).

151. The fusion protein of any of embodiments 119-144, 149, and 150, wherein the fusion protein comprises the sequence set forth in SEQ ID NO:71, or an amino acid sequence that has at least 90%, 91%, 92%, 93%, 94%, 95%, 96%, 97%, 98%, or 99% sequence identity thereto.

152. The fusion protein of any of embodiments 119-144, 149, and 150, wherein the fusion protein comprises the sequence set forth in SEQ ID NO:77, or an amino acid sequence that has at least 90%, 91%, 92%, 93%, 94%, 95%, 96%, 97%, 98%, or 99% sequence identity thereto.

153. A combination comprising the fusion protein of any of embodiments 119-152 and at least one gRNA, optionally wherein the at least one gRNA is a gRNA of any of embodiments 91-109.

154. A polynucleotide encoding the DNA-targeting system of any of embodiments 1-90, the gRNA of any of embodiments 91-109, the combination of any of embodiments 110-118 and 153, or the fusion protein of any of embodiments 119-152, or a portion or a component of any of the foregoing.

155. A polynucleotide encoding a first DNA-targeting system, a first Cas protein and/or a first gRNA of the DNA-targeting system of any of embodiments 56-90 or the combination of any of embodiments 110-118 and 153.

156. A polynucleotide encoding a second DNA-targeting system, a second Cas protein and/or a second gRNA of the DNA-targeting system of any of embodiments 56-90 or the combination of any of embodiments 110-118 and 153.

157. A plurality of polynucleotides, comprising the polynucleotide of any of embodiments 154-156, and one or more additional polynucleotides encoding an additional portion or an additional component of the DNA-targeting system of any of embodiments 1-90, the gRNA of any of embodiments 91-109, the combination of any of embodiments 110-118 and 153, or the fusion protein of any of embodiments 119-152, or a portion or a component of any of the foregoing.

158. A plurality of polynucleotides, comprising:
a first polynucleotide comprising the polynucleotide of embodiment 155; and
a second polynucleotide comprising the polynucleotide of embodiment 156.

159. A vector comprising the polynucleotide of any of embodiments 154-156, the plurality of polynucleotides of embodiment 157 or 158, or a first polynucleotide or a second polynucleotide of the plurality of polynucleotides of embodiment 157 or 158, or a portion or a component of any of the foregoing.

160. The vector of embodiment 158, wherein the vector is a viral vector, optionally wherein the viral vector is an AAV vector.

161. The vector of embodiment 160, wherein the viral vector, optionally the AAV vector, exhibits central nervous system (CNS) tropism.

162. The vector of embodiment 160 or 161, wherein the viral vector is an AAV vector and the AAV vector is selected from among AAV1, AAV2, AAV3, AAV4, AAV5, AAV6, AAV7, AAV8, or AAV9 vector, optionally an AAV5 vector or an AAV9 vector, optionally an AAV9 vector.

163. The vector of embodiment 159, wherein the vector is a non-viral vector selected from: a lipid nanoparticle, a liposome, an exosome, or a cell penetrating peptide

164. A plurality of vectors, comprising the vector of any of embodiments 159-163, and one or more additional vectors comprising one or more additional polynucleotides encoding an additional portion or an additional component of the DNA-targeting system of any of embodiments 1-90, the gRNA of any of embodiments 91-109, the combination of any of embodiments 110-118 and 153, or the fusion protein of any of embodiments 119-152, or a portion or a component of any of the foregoing.

165. A plurality of vectors, comprising:
a first vector comprising the polynucleotide of embodiment 154; and
a second vector comprising the polynucleotide of embodiment 155.

166. A cell comprising the DNA-targeting system of any of embodiments 1-90, the gRNA of any of embodiments 91-109, the combination of any of embodiments 110-118 and 153, the fusion protein of any of embodiments 119-152, the polynucleotide of any of embodiments 154-156, the plurality of polynucleotides of embodiment 157 or 158, the vector of any of embodiments 159-163, the plurality of vectors of embodiment 164 or 165, or a portion or a component of any of the foregoing.

167. The cell of embodiment 166, wherein the cell is a heart cell, a skeletal muscle cell, a nervous system cell, or an induced pluripotent stem cell.

168. The cell of embodiment 166 or 167, wherein the cell is from a subject that has or is suspected of having Friedreich's ataxia (FA).

169. A method for modulating the expression of frataxin (FXN) in a cell, the method comprising: introducing the DNA-targeting system of any of embodiments 1-90, the gRNA of any of embodiments 91-109, the combination of any of embodiments 110-118 and 153, the fusion protein of any of embodiments 119-152, the polynucleotide of any of embodiments 154-156, the plurality of polynucleotides of embodiment 157 or 158, the vector of any of embodiments 159-163, the plurality of vectors of embodiment 164 or 165, or a portion or a component of any of the foregoing, into the cell.

170. A method of inducing a genetic disruption at a target site in an enhancer region of a frataxin (FXN) locus in a cell, wherein the target site is located within the genomic coordinates human genome assembly GRCh38 (hg38) chr9:69,027,282-69,028,497, the method comprising:
contacting a cell with the DNA-targeting system of any of embodiments 1, 3, 4, 7-9, 11, 14, 17-52, 55-73, and 77-90, the gRNA of any of embodiments 91-109, the combination of any of embodiments 110-118 and 153, the fusion protein of any of embodiments 119, 121, 122, 124, 125, 127, 129, 133-150, the polynucleotide of any of embodiments 154-156, the plurality of polynucleotides of embodiment 157 or 158, the vector of any of embodiments 159-163, the plurality of vectors of embodiment 164 or 165, or a portion or a component of any of the foregoing.

171. The method of embodiment 169 or 170, wherein the cell is from a subject that has or is suspected of having Friedreich's ataxia (FA).

172. A method for modulating the expression of frataxin (FXN) in a subject, the method comprising:
administering the DNA-targeting system of any of embodiments 1-90, the gRNA of any of embodiments 91-109, the combination of any of embodiments 110-118 and 153, the fusion protein of any of embodiments 119-152, the polynucleotide of any of embodiments 154-156, the plurality of polynucleotides of embodiment 157 or 158, the vector of any of embodiments 159-163, the plurality of vectors of embodiment 164 or 165, or a portion or a component of any of the foregoing, to the subject.

173. The method of embodiment 171 or 172, wherein the subject has or is suspected of having Friedreich's ataxia (FA).

174. A method of treating Friedreich's ataxia (FA), the method comprising:
administering the DNA-targeting system of any of embodiments 1-90, the gRNA of any of embodiments 91-109, the combination of any of embodiments 110-118 and 153, the fusion protein of any of embodiments 119-152, the polynucleotide of any of embodiments 154-156, the plurality of polynucleotides of embodiment 157 or 158, the vector of any of embodiments 159-163, the plurality of vectors of embodiment 164 or 165, or a portion or a component of any of the foregoing, to a subject that has or is suspected of having FA.

175. The method of any of embodiments 171-174, wherein a cell in the subject exhibits reduced expression of FXN compared to a cell from a normal subject.

176. The method of any of embodiments 171-175, wherein a cell in the subject has a GAA trinucleotide repeat expansion in the FXN gene.

177. The method of any of embodiments 169-176, wherein the cell is a heart cell, a skeletal muscle cell, a nervous system cell, or an induced pluripotent stem cell.

178. The method of any of embodiments 169-177, wherein the introducing, contacting or administering is carried out in vivo or ex vivo.

179. The method of any of embodiments 169-178, wherein following the introducing, contacting or administering, the expression of frataxin (FXN) is increased in the cell or the subject.

180. The method of embodiment 179, wherein the expression is increased at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold.

181. The method of embodiment 179 or 180, wherein the expression is increased by less than about 10-fold, 9-fold, 8-fold, 7-fold or 6-fold.

182. The method of any of embodiments 171-181, wherein the subject is a human.

183. A pharmaceutical composition comprising the DNA-targeting system of any of embodiments 1-90, the gRNA of any of embodiments 91-109, the combination of any of embodiments 110-118 and 153, the fusion protein of any of embodiments 119-152, the polynucleotide of any of embodiments 154-156, the plurality of polynucleotides of embodiment 157 or 158, the vector of any of embodiments 159-163, the plurality of vectors of embodiment 164 or 165, or a portion or a component of any of the foregoing.

184. The pharmaceutical composition of embodiment 183, for use in treating Friedreich's ataxia (FA).

185. The pharmaceutical composition of embodiment 183, for use in the manufacture of a medicament for treating Friedreich's ataxia (FA).

186. The pharmaceutical composition for use of embodiment 184 or 185, wherein the pharmaceutical composition is to be administered to a subject.

187. Use of the pharmaceutical composition of embodiment 183 for treating Friedreich's ataxia (FA).

188. Use of the pharmaceutical composition of embodiment 183 in the manufacture of a medicament for treating Friedreich's ataxia (FA).

189. The use of embodiment 187 or 188, wherein the pharmaceutical composition is to be administered to a subject.

190. The pharmaceutical composition for use or the use of any of embodiments 184-189, wherein the subject has or is suspected of having Friedreich's ataxia (FA).

191. The pharmaceutical composition for use or the use of any of embodiments 184-190, wherein a cell in the subject exhibits reduced expression of FXN compared to a cell from a normal subject.

192. The pharmaceutical composition for use or the use of any of embodiments 184-191, wherein a cell in the subject has a GAA trinucleotide repeat expansion in the FXN gene.

193. The pharmaceutical composition for use or the use of embodiment 191 or 192, wherein the cell is a heart cell, a skeletal muscle cell, a nervous system cell, or an induced pluripotent stem cell.

194. The pharmaceutical composition for use or the use of any of embodiments 186 and 189-193, wherein the administration is carried out in vivo or ex vivo.

195. The pharmaceutical composition for use or the use of any of embodiments 186 and 189-194, wherein following the administration, the expression of frataxin (FXN) is increased in the cell or the subject.

196. The pharmaceutical composition for use or the use of embodiment 195, wherein the expression is increased at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold.

197. The pharmaceutical composition for use or the use of embodiment 195 or 196, wherein the expression is increased by less than about 10-fold, 9-fold, 8-fold, 7-fold or 6-fold.

198. The pharmaceutical composition for use or the use of any of embodiments 186 and 189-197, wherein the subject is a human.

201. A DNA-targeting system comprising:
(a) a DNA-targeting domain that binds to a target site in a regulatory DNA element of a frataxin (FXN) locus; and
(b) at least one effector domain that increases transcription of the FXN locus.

202. A DNA-targeting system comprising: a DNA-targeting domain that binds to a target site in an enhancer of a frataxin (FXN) locus.

203. A DNA-targeting system comprising:
(a) a DNA-targeting domain that binds to a target site in an enhancer of a frataxin (FXN) locus; and
(b) at least one effector domain that increases transcription of the FXN locus.

204. The DNA-targeting system of any of embodiments 201-203, wherein binding of the DNA-targeting domain to the target site does not introduce a genetic disruption or a DNA break at or near the target site.

205. The DNA-targeting system of any of embodiments 201-204, wherein the DNA-targeting domain comprises a Clustered Regularly Interspaced Short Palindromic Repeats associated (Cas)-guide RNA (gRNA) combination comprising (a) a Cas protein or a variant thereof and (b) at least one gRNA; a zinc finger protein (ZFP); a transcription activator-like effector (TALE); a meganuclease; a homing endonuclease; or an I-SceI enzyme or a variant thereof, optionally wherein the DNA-targeting domain comprises a catalytically inactive variant of any of the foregoing.

206. The DNA-targeting system of any of embodiments 201-205, wherein the DNA-targeting domain comprises a Cas-gRNA combination comprising (a) a Cas protein or a variant thereof and (b) at least one gRNA.

207. The DNA-targeting system of embodiment 5 or 6, wherein the Cas protein or a variant thereof is a deactivated Cas (dCas) protein.

208. A DNA-targeting system comprising a DNA-targeting domain that is a Cas-guide RNA (gRNA) combination comprising:
(a) a deactivated Cas (dCas) protein;
(b) at least one effector domain that increases transcription of a frataxin (FXN) locus; and
(c) at least one gRNA comprising a gRNA spacer sequence that is capable of hybridizing to a target site in a regulatory DNA element of the FXN locus or is complementary to the target site.

209. The DNA-targeting system of any of embodiments 201-208, wherein at least one gRNA is capable of complexing with the Cas protein or variant thereof.

10. The DNA-targeting system of any of embodiments 201-209, wherein at least one gRNA comprises a gRNA spacer sequence that is capable of hybridizing to the target site or is complementary to the target site.

211. The DNA-targeting system of any of embodiments 201-210, wherein the Cas protein or a variant thereof is a Cas9 protein or a variant thereof.

212. The DNA-targeting system of any of embodiments 201-212, wherein the Cas protein or a variant thereof is a deactivated Cas9 (dCas9) protein.

213. The DNA-targeting system of embodiment 211 or 212, wherein the Cas9 protein or a variant thereof is a Staphylococcus aureus Cas9 (SaCas9) protein or a variant thereof.

214. A DNA-targeting system comprising a DNA-targeting domain that is a Cas-guide RNA (gRNA) combination comprising:
(a) Staphylococcus aureus dCas9 protein (dSaCas9);
(b) at least one effector domain that increases transcription of a frataxin (FXN) locus; and
(c) at least one gRNA, comprising a gRNA spacer sequence that is capable of hybridizing to a target site in a regulatory DNA element of the FXN locus or is complementary to the target site.

215. A DNA-targeting system comprising a DNA-targeting domain that is a Cas-guide RNA (gRNA) combination comprising:
(a) Staphylococcus aureus dCas9 protein (dSaCas9); and
(b) at least one gRNA, comprising a gRNA spacer sequence that is capable of hybridizing to a target site in an enhancer of a frataxin (FXN) locus or is complementary to the target site.

216. A DNA-targeting system comprising a DNA-targeting domain that is a Cas-guide RNA (gRNA) combination comprising:
(a) Staphylococcus aureus dCas9 protein (dSaCas9);
(b) at least one effector domain that increases transcription of a frataxin (FXN) locus; and
(c) at least one gRNA, comprising a gRNA spacer sequence that is capable of hybridizing to a target site in an enhancer of the FXN locus or is complementary to the target site.

217. The DNA-targeting system of any of embodiments 201-216, wherein the Cas protein or a variant thereof is a Staphylococcus aureus dCas9 protein (dSaCas9) that comprises at least one amino acid mutation selected from D10A and N580A, with reference to numbering of positions of SEQ ID NO:73.

218. The DNA-targeting system of any of embodiments 201-217, wherein the Cas protein or a variant thereof comprises the sequence set forth in SEQ ID NO:72, or an amino acid sequence that has at least 90%, 91%, 92%, 93%, 94%, 95%, 96%, 97%, 98%, or 99% sequence identity thereto.

219. The DNA-targeting system of any of embodiments 201-216, wherein the Cas protein or a variant thereof is a Streptococcus pyogenes Cas9 (SpCas9) protein or a variant thereof.

220. The DNA-targeting system of any of embodiments 201-216 and 219, wherein the Cas protein or a variant thereof is a Streptococcus pyogenes dCas9 (dSpCas9) protein that comprises at least one amino acid mutation selected from D10A and H840A, with reference to numbering of positions of SEQ ID NO:79.

221. The DNA-targeting system of any of embodiments 201-216, 219, and 220, wherein the Cas protein or a variant thereof comprises the sequence set forth in SEQ ID NO:78, or an amino acid sequence that has at least 90%, 91%, 92%, 93%, 94%, 95%, 96%, 97%, 98%, or 99% sequence identity thereto.

222. The DNA-targeting system of any of embodiments 201-221, wherein the Cas protein or a variant thereof is a split variant Cas protein, wherein the split variant Cas protein comprises a first polypeptide comprising an N-terminal fragment of the variant Cas protein and an N-terminal Intein, and a second polypeptide comprising a C-terminal fragment of the variant Cas protein and a C-terminal Intein.

223. The DNA-targeting system of embodiment 222, wherein when the first polypeptide and the second polypeptide of the split variant Cas protein are present in proximity or present in the same cell, the N-terminal Intein and C-terminal Intein self-excise and ligate the N-terminal fragment and the C-terminal fragment of the variant Cas protein to form a full-length variant Cas protein.

224. The DNA-targeting system of embodiment 222 or 223, wherein the N-terminal Intein comprises an N-terminal Npu Intein, or the sequence set forth in SEQ ID NO:178, or an amino acid sequence that has at least 90%, 91%, 92%, 93%, 94%, 95%, 96%, 97%, 98%, or 99% sequence identity thereto, or a portion of any of the foregoing.

225. The DNA-targeting system of any of embodiments 222-224, wherein the N-terminal fragment of the variant Cas protein comprises:
the N-terminal fragment of variant SpCas9 from the N-terminal end up to position 573 of the dSpCas9 sequence set forth in SEQ ID NO:78, or an amino acid sequence that has at least 90%, 91%, 92%, 93%, 94%, 95%, 96%, 97%, 98%, or 99% sequence identity thereto; or
the sequence set forth in SEQ ID NO:176, or an amino acid sequence that has at least 90%, 91%, 92%, 93%, 94%, 95%, 96%, 97%, 98%, or 99% sequence identity thereto, or a portion of any of the foregoing.

226. The DNA-targeting system of any of embodiments 222-225, wherein the C-terminal Intein comprises a C-terminal Npu Intein, or the sequence set forth in SEQ ID NO:182, or an amino acid sequence that has at least 90%, 91%, 92%, 93%, 94%, 95%, 96%, 97%, 98%, or 99% sequence identity thereto, or a portion of any of the foregoing.

227. The DNA-targeting system of any of embodiments 222-226, wherein the C-terminal fragment of the variant Cas protein comprises:
the C-terminal fragment of variant SpCas9 from position 574 to the C-terminal end of the dSpCas9 sequence set forth in SEQ ID NO:78, or an amino acid sequence that has at least 90%, 91%, 92%, 93%, 94%, 95%, 96%, 97%, 98%, or 99% sequence identity thereto; or
the sequence set forth in SEQ ID NO:184, or an amino acid sequence that has at least 90%, 91%, 92%, 93%, 94%, 95%, 96%, 97%, 98%, or 99% sequence identity thereto, or a portion of any of the foregoing.

228. The DNA-targeting system of any of embodiments 201-227, wherein the regulatory DNA element is an enhancer.

229. The DNA-targeting system of any of embodiments 201-228, wherein the target site is located within the genomic coordinates human genome assembly GRCh38 (hg38) chr9:69,027,282-69,028,497.

230. The DNA-targeting system of any of embodiments 201-229, wherein the target site is located within the genomic coordinates hg38 chr9:69,027,615-69,028,101.

231. A DNA-targeting system comprising:
(a) a DNA-targeting domain that binds to a target site in a regulatory DNA element of a frataxin (FXN) locus; and
(b) at least one effector domain that increases transcription of the FXN locus;
wherein the target site is located within the genomic coordinates human genome assembly GRCh38 (hg38) chr9:69,027,282-69,028,497.

232. The DNA-targeting system of any of embodiments 201-231, wherein the target site comprises the sequence set forth in SEQ ID NO:21, a contiguous portion thereof of at least 14 nt, or a complementary sequence of any of the foregoing.

233. The DNA-targeting system of any of embodiments 201-232, wherein the at least one gRNA comprises a gRNA spacer sequence comprising the sequence set forth in SEQ ID NO:42, or a contiguous portion thereof of at least 14 nt.

234. The DNA-targeting system of any of embodiments 201-233, wherein the at least one gRNA further comprises the sequence set forth in SEQ ID NO:44.

235. The DNA-targeting system of any of embodiments 201-234, wherein the at least one gRNA comprises a gRNA that comprises the sequence set forth in SEQ ID NO:67, optionally wherein the at least one gRNA is the gRNA sequence set forth in SEQ ID NO:67.

236. The DNA-targeting system of any of embodiments 201 and 204-227, wherein the regulatory DNA element is a promoter.

237. The DNA-targeting system of any of embodiments 201, 204-227, and 236, wherein the target site is located within the genomic coordinates hg38 chr9:68,940,179-69,205,519.

238. The DNA-targeting system of any of embodiments 201, 204-227, 236, and 237, wherein the target site comprises a sequence selected from any of SEQ ID NOS: 1-10, a contiguous portion thereof of at least 14 nt, or a complementary sequence of any of the foregoing.

239. The DNA-targeting system of any of embodiments 201-227 and 236-238, wherein the at least one gRNA comprises a gRNA spacer sequence comprising a sequence selected from any of SEQ ID NOS:22-31, or a contiguous portion thereof of at least 14 nt.

240. The DNA-targeting system of any of embodiments 201-227 and 236-239, wherein the at least one gRNA comprises a gRNA spacer sequence comprising SEQ ID NO:22, or a contiguous portion thereof of at least 14 nt.

241. The DNA-targeting system of any of embodiments 201-227 and 236-239, wherein the at least one gRNA comprises a gRNA spacer sequence comprising SEQ ID NO:23, or a contiguous portion thereof of at least 14 nt.

242. The DNA-targeting system of any of embodiments 201-227 and 236-239, wherein the at least one gRNA comprises a gRNA spacer sequence comprising SEQ ID NO:24, or a contiguous portion thereof of at least 14 nt.

243. The DNA-targeting system of any of embodiments 201-227 and 236-239, wherein the at least one gRNA comprises a gRNA spacer sequence comprising SEQ ID NO:25, or a contiguous portion thereof of at least 14 nt.

244. The DNA-targeting system of any of embodiments 201-227 and 236-239, wherein the at least one gRNA comprises a gRNA spacer sequence comprising SEQ ID NO:26, or a contiguous portion thereof of at least 14 nt.

245. The DNA-targeting system of any of embodiments 201-227 and 236-239, wherein the at least one gRNA comprises a gRNA spacer sequence comprising SEQ ID NO:27, or a contiguous portion thereof of at least 14 nt.

246. The DNA-targeting system of any of embodiments 201-227 and 236-239, wherein the at least one gRNA comprises a gRNA spacer sequence comprising SEQ ID NO:28, or a contiguous portion thereof of at least 14 nt.

247. The DNA-targeting system of any of embodiments 201-227 and 236-239, wherein the at least one gRNA comprises a gRNA spacer sequence comprising SEQ ID NO:29, or a contiguous portion thereof of at least 14 nt.

248. The DNA-targeting system of any of embodiments 201-227 and 236-239, wherein the at least one gRNA comprises a gRNA spacer sequence comprising SEQ ID NO:30, or a contiguous portion thereof of at least 14 nt.

249. The DNA-targeting system of any of embodiments 201-227 and 236-239, wherein the at least one gRNA comprises a gRNA spacer sequence comprising SEQ ID NO:31, or a contiguous portion thereof of at least 14 nt.

250. The DNA-targeting system of any of embodiments 201-227 and 236-249, wherein the gRNA further comprises the sequence set forth in SEQ ID NO:44.

251. The DNA-targeting system of any of embodiments 201-227, 236-239, and 250, wherein the at least one gRNA comprises a gRNA that comprises a sequence selected from any of SEQ ID NOS:47-56, optionally wherein the at least one gRNA is the gRNA sequence set forth in any of SEQ ID NOS:47-56, optionally wherein the gRNA is set forth in SEQ ID NO:47 or 53.

252. The DNA-targeting system of any of embodiments 201, 204-227, 236, and 237, wherein the target site comprises a sequence selected from any of SEQ ID NOS:11-20, a contiguous portion thereof of at least 14 nt, or a complementary sequence of any of the foregoing.

253. The DNA-targeting system of any of embodiments 201-227, 236, 237, and 252, wherein the at least one gRNA comprises a gRNA spacer sequence comprising a sequence selected from any of SEQ ID NOS:32-41, or a contiguous portion thereof of at least 14 nt.

254. The DNA-targeting system of any of embodiments 201, 204-227, 236, 237, 252, and 253, wherein the target site comprises a sequence selected from any of SEQ ID NOS: 12-14 and 16-19, a contiguous portion thereof of at least 14 nt, or a complementary sequence of any of the foregoing.

255. The DNA-targeting system of any of embodiments 201-227, 236, 237, and 252-254, wherein the at least one gRNA comprises a gRNA spacer sequence comprising a sequence selected from any of SEQ ID NOS:33-35 and 37-40, or a contiguous portion thereof of at least 14 nt.

256. The DNA-targeting system of any of embodiments 201-227, 236, 237 and 252-255, wherein the gRNA further comprises the sequence set forth in SEQ ID NO:46.

257. The DNA-targeting system of any of embodiments 201-227, 236, 237, and 252-256, wherein the at least one gRNA comprises a gRNA that comprises a sequence selected from any of SEQ ID NOS:57-66, optionally wherein the at least one gRNA is the gRNA sequence set forth in any of SEQ ID NOS:57-66.

258. The DNA-targeting system of any of embodiments 208-257, wherein the gRNA spacer sequence is between 14 nt and 24 nt, or between 16 nt and 22 nt in length.

259. The DNA-targeting system of any of embodiments 208-258, wherein the gRNA spacer sequence is 18 nt, 19 nt, 20 nt, 21 nt or 22 nt in length.

260. The DNA-targeting system of any of embodiments 201-259, wherein the gRNA comprises modified nucleotides for increased stability.

261. The DNA-targeting system of any of embodiments 201-260, wherein the DNA-targeting domain or a component thereof is fused to the at least one effector domain.

262. The DNA-targeting system of embodiment 261, wherein the DNA-targeting domain comprises a Cas-gRNA combination comprising (a) a Cas protein or a variant thereof and (b) at least one gRNA, and the component thereof fused to the at least one effector domain is the Cas protein or a variant thereof.

263. The DNA-targeting system of any of embodiments 201-262, wherein the effector domain induces transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation.

264. The DNA-targeting system of any of embodiments 201-263, wherein the effector domain induces transcription activation, transcription co-activation, transcription elongation.

265. The DNA-targeting system of any of embodiments 201-264, wherein the effector domain induces transcription activation.

266. A DNA-targeting system comprising a DNA-targeting domain that is a Cas-guide RNA (gRNA) combination comprising:
(a) a Staphylococcus aureus deactivated Cas9 protein (dSaCas9) protein set forth in SEQ ID NO:72 fused to at least one effector domain that increases transcription of the FXN locus; and
(b) a gRNA comprising a gRNA spacer sequence set forth in SEQ ID NO:42.

267. A DNA-targeting system comprising a DNA-targeting domain that is a Cas-guide RNA (gRNA) combination comprising:
(a) a Staphylococcus aureus deactivated Cas9 protein (dSaCas9) protein set forth in SEQ ID NO:72 fused to at least one effector domain that increases transcription of the FXN locus; and
(b) a gRNA comprising a gRNA spacer sequence set forth in SEQ ID NO:22.

268. A DNA-targeting system comprising a DNA-targeting domain that is a Cas-guide RNA (gRNA) combination comprising:
(a) a Staphylococcus aureus deactivated Cas9 protein (dSaCas9) protein set forth in SEQ ID NO:72 fused to at least one effector domain that increases transcription of the FXN locus; and
(b) a gRNA comprising a gRNA spacer sequence set forth in SEQ ID NO:28.

269. The DNA-targeting system of any of embodiments 201-268, wherein the effector domain comprises at least one VP16 domain, or a VP16 tetramer (“VP64”) or a variant thereof.

270. A DNA-targeting system comprising a DNA-targeting domain that is a Cas-guide RNA (gRNA) combination comprising:
(a) a Staphylococcus aureus deactivated Cas9 protein (dSaCas9) protein set forth in SEQ ID NO:72 fused to a VP16 tetramer (“VP64”); and
(b) a gRNA comprising a gRNA spacer sequence set forth in SEQ ID NO:42.

271. A DNA-targeting system comprising a DNA-targeting domain that is a Cas-guide RNA (gRNA) combination comprising:
(a) a Staphylococcus aureus deactivated Cas9 protein (dSaCas9) protein set forth in SEQ ID NO:72 fused to a VP16 tetramer (“VP64”); and
(b) a gRNA comprising a gRNA spacer sequence set forth in SEQ ID NO:22.

272. A DNA-targeting system comprising a DNA-targeting domain that is a Cas-guide RNA (gRNA) combination comprising:
(a) a Staphylococcus aureus deactivated Cas9 protein (dSaCas9) protein set forth in SEQ ID NO:72 fused to a VP16 tetramer (“VP64”); and
(b) a gRNA comprising a gRNA spacer sequence set forth in SEQ ID NO:28.

273. The DNA-targeting system of any of embodiments 201-272, wherein the effector domain comprises the sequence set forth in SEQ ID NO:81 or 83, or a portion thereof, or an amino acid sequence that has at least 90%, 91%, 92%, 93%, 94%, 95%, 96%, 97%, 98%, or 99% sequence identity to any of the foregoing.

274. The DNA-targeting system of any of embodiments 201-265, wherein the effector domain is selected from a p65 activation domain, a p300 domain, DPOLA, ENL, FOXO3, HSH2D, NCOA2, NCOA3, PSA1, PYGO1, RBM39, HERC2, DMD, or NOTCH2, or a domain thereof, a portion thereof or a variant thereof.

275. The DNA-targeting system of embodiment 274, wherein the effector domain comprises a sequence selected from any of SEQ ID NOS: 100-112, or a domain thereof, a portion thereof, or an amino acid sequence that has at least 90%, 91%, 92%, 93%, 94%, 95%, 96%, 97%, 98%, or 99% sequence identity to any of the foregoing.

276. The DNA-targeting system of embodiment 274 or 275, wherein the effector domain comprises a sequence selected from any of SEQ ID NOS:113-125, or a domain thereof, a portion thereof, or an amino acid sequence that has at least 90%, 91%, 92%, 93%, 94%, 95%, 96%, 97%, 98%, or 99% sequence identity to any of the foregoing.

277. The DNA-targeting system of any of embodiments 274-276, wherein the variant thereof comprises a truncation thereof.

278. The DNA-targeting system of any of embodiments 261-277, wherein the at least one effector domain is fused to the N-terminus, the C-terminus, or both the N-terminus and the C-terminus, of the DNA-targeting domain or a component thereof.

279. The DNA-targeting system of any of embodiments 201-278, further comprising one or more linkers connecting the DNA-targeting domain or a component thereof to the at least one effector domain, and/or further comprising one or more nuclear localization signals (NLS).

280. The DNA-targeting system of any of embodiments 201-273, 278 and 279, wherein the DNA-targeting system comprises the sequence set forth in SEQ ID NO:71, or an amino acid sequence that has at least 90%, 91%, 92%, 93%, 94%, 95%, 96%, 97%, 98%, or 99% sequence identity thereto.

281. The DNA-targeting system of any of embodiments 201-273, 278 and 279, wherein the DNA-targeting system comprises the sequence set forth in SEQ ID NO:77, or an amino acid sequence that has at least 90%, 91%, 92%, 93%, 94%, 95%, 96%, 97%, 98%, or 99% sequence identity thereto.

282. A combination, comprising:
a first DNA-targeting domain comprising the DNA targeting domain of any of embodiments 201-281, and
one or more second DNA-targeting domains, optionally wherein the one or more second DNA-targeting domains comprises the DNA targeting domain of any of embodiments 201-281.

283. The combination of embodiment 82, wherein:
the first DNA-targeting domain binds a first target site in an enhancer of a FXN locus; and
the second DNA-targeting domain binds a second target site in a promoter of a FXN locus.

284. A combination comprising:
a first DNA-targeting domain that binds a first target site in an enhancer of a frataxin (FXN) locus; and
a second DNA-targeting domain that binds a second target site in a promoter of a FXN locus.

285. The combination of embodiment 283 or 284, wherein:
the first target site is located within the genomic coordinates human genome assembly GRCh38 (hg38) chr9:69,027,282-69,028,497; and
the second target site is located within the genomic coordinates hg38 chr9:68,940, 179-69,205,519.

286. The combination of any of embodiments 283-285, wherein:
the first target site is located within the genomic coordinates hg38 chr9:69,027,615-69,028,101; and
the second target site is located within the genomic coordinates hg38 chr9:68,940,179-69,205,519.

287. The combination of any of embodiments 282-286, wherein:
the first DNA-targeting domain comprises a first Cas-gRNA combination comprising (a) a first Cas protein or a variant thereof and (b) a first gRNA that is capable of hybridizing to the target site or is complementary to the first target site; and
the second DNA-targeting domain comprises a second Cas-gRNA combination comprising (a) a second Cas protein or a variant thereof and (b) a second gRNA that is capable of hybridizing to the target site or is complementary to the second target site.

288. The combination of any of embodiments 282-287, wherein:
the first DNA-targeting domain comprises a first Cas-gRNA combination comprising (a) a first Cas protein or a variant thereof and (b) a first gRNA comprising a gRNA spacer sequence set forth in SEQ ID NO:42 or a contiguous portion thereof of at least 14 nt.

289. The combination of any of embodiments 282-288, wherein:
the second DNA-targeting domain comprises a second Cas-gRNA combination comprising (a) a second Cas protein or a variant thereof and (b) a second gRNA comprising a gRNA spacer sequence set forth in SEQ ID NO:22 or 28 or a contiguous portion thereof of at least 14 nt.

290. The combination of any of embodiments 282-289, wherein:
the first Cas-gRNA combination comprises (a) a first Cas protein or a variant thereof and (b) a first gRNA comprising a gRNA spacer sequence set forth in SEQ ID NO:42 or a contiguous portion thereof of at least 14 nt; and
the second Cas-gRNA combination comprises (a) a second Cas protein or a variant thereof and (b) a second gRNA comprising a gRNA spacer sequence set forth in SEQ ID NO:22 or a contiguous portion thereof of at least 14 nt.

291. The combination of any of embodiments 282-289, wherein:
the first Cas-gRNA combination comprises (a) a first Cas protein or a variant thereof and (b) a first gRNA comprising a gRNA spacer sequence set forth in SEQ ID NO:42 or a contiguous portion thereof of at least 14 nt; and
the second Cas-gRNA combination comprises (a) a second Cas protein or a variant thereof and (b) a second gRNA comprising a gRNA spacer sequence set forth in SEQ ID NO:28 or a contiguous portion thereof of at least 14 nt.

292. The combination of embodiment 282, wherein:
the first DNA-targeting domain binds a first target site in a promoter of a FXN locus; and
the second DNA-targeting domain binds a second target site in a promoter of a FXN locus.

293. A combination comprising:
a first DNA-targeting domain that binds a first target site in a promoter of a frataxin (FXN) locus; and
a second DNA-targeting domain that binds a second target site in a promoter of a FXN locus.

294. The combination of any of embodiments 282, 292, and 293, wherein the first target site and the second target site independently are located within the genomic coordinates hg38 chr9:68,940,179-69,205,519.

295. The combination of any of embodiments 282 and 292-294, wherein the first target site and the second target site are different.

296. The combination of any of embodiments 282 and 292-295, wherein:
the first DNA-targeting domain comprises a first Cas-gRNA combination comprising (a) a first Cas protein or a variant thereof and (b) a first gRNA that is capable of hybridizing to the target site or is complementary to the first target site; and
the second DNA-targeting domain comprises a second Cas-gRNA combination comprising (a) a second Cas protein or a variant thereof and (b) a second gRNA that is capable of hybridizing to the target site or is complementary to the second target site.

297. The combination of any of embodiments 282 and 292-296, wherein:
the first DNA-targeting domain comprises a first Cas-gRNA combination comprising (a) a first Cas protein or a variant thereof and (b) a first gRNA comprising a gRNA spacer sequence set forth in SEQ ID NO:22 or a contiguous portion thereof of at least 14 nt.

298. The combination of any of embodiments 282 and 292-297, wherein:
the second DNA-targeting domain comprises a second Cas-gRNA combination comprising (a) a second Cas protein or a variant thereof and (b) a second gRNA comprising a gRNA spacer sequence set forth in SEQ ID NO:28 or a contiguous portion thereof of at least 14 nt.

299. The combination of any of embodiments 282 and 292-298, wherein:
the first Cas-gRNA combination comprises (a) a first Cas protein or a variant thereof and (b) a first gRNA comprising a gRNA spacer sequence set forth in SEQ ID NO:22 or a contiguous portion thereof of at least 14 nt; and
the second Cas-gRNA combination comprises (a) a second Cas protein or a variant thereof and (b) a second gRNA comprising a gRNA spacer sequence set forth in SEQ ID NO:28 or a contiguous portion thereof of at least 14 nt.

300. The combination of any of embodiments 287-292 and 296-299, wherein the first Cas protein or a variant thereof and/or the second Cas protein or a variant thereof is a deactivated Cas9 (dCas9) protein.

301. The combination of embodiment 300, wherein the first variant Cas protein and/or the second variant Cas protein is a Staphylococcus aureus dCas9 protein (dSaCas9) that comprises at least one amino acid mutation selected from D10A and N580A, with reference to numbering of positions of SEQ ID NO:73; or comprises the sequence set forth in SEQ ID NO:72, or an amino acid sequence that has at least 290%, 291%, 292%, 293%, 294%, 295%, 296%, 297%, 298%, or 299% sequence identity thereto.

302. The combination of embodiment 300 or 301, wherein the first variant Cas protein and/or the second variant Cas protein is a Streptococcus pyogenes dCas9 (dSpCas9) protein that comprises at least one amino acid mutation selected from D10A and H840A, with reference to numbering of positions of SEQ ID NO:79; or comprises the sequence set forth in SEQ ID NO:78, or an amino acid sequence that has at least 290%, 291%, 292%, 293%, 294%, 295%, 296%, 297%, 298%, or 299% sequence identity thereto.

303. The combination of any of embodiments 287-292 and 296-302, wherein the first variant Cas protein and/or the second variant Cas protein is a split variant Cas9 protein, wherein the split Cas9 protein comprises a first polypeptide comprising an N-terminal fragment of the variant Cas9 and an N-terminal Intein, and a second polypeptide comprising a C-terminal fragment of the variant Cas9 and a C-terminal Intein.

304. The combination of any of embodiments 287-292 and 296-303, wherein the first Cas protein and the second Cas protein are the same.

305. The combination of any of embodiments 287-292 and 296-303, wherein the first Cas protein and the second Cas protein are different.

306. The combination of any of embodiments 287-292 and 296-305, wherein the first Cas protein or a variant thereof and/or the second Cas protein or a variant thereof is fused to at least one effector domain.

307. The combination of embodiment 306, wherein the effector domain induces transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation.

308. The combination of embodiment 306 or 307, wherein the effector domain induces transcription activation.

309. The combination of any of embodiments 282-308, wherein the first DNA-targeting domain and the second DNA-targeting domain are encoded in a first polynucleotide.

310. The combination of any of embodiments 282-309, wherein the first Cas protein and the second Cas protein are encoded in a first polynucleotide.

311. The combination of any of embodiments 282-310, wherein the first Cas protein and the second Cas protein are encoded by the same nucleotide sequence.

312. The combination of any of embodiments 282-311, wherein the first gRNA and the second gRNA are encoded in a first polynucleotide.

313. The combination of any of embodiments 282-312, wherein the first Cas protein and the second Cas protein are encoded by the same nucleotide sequence, and the Cas protein, the first gRNA, and the second gRNA are encoded in a first polynucleotide.

314. The combination of any of embodiments 282-308, wherein the first DNA-targeting domain is encoded in a first polynucleotide and the second DNA-targeting domain is encoded in a second polynucleotide.

315. The combination of any of embodiments 282-308 and 314, wherein the first Cas protein is encoded in a first polynucleotide and the second Cas protein is encoded in a second polynucleotide.

316. The combination of any of embodiments 282-308, 314, and 315, wherein the first gRNA is encoded in a first polynucleotide and the second gRNA is encoded in a second polynucleotide.

317. The combination of any of embodiments 282-308 and 314-316, wherein the first Cas protein and the first gRNA are encoded in a first polynucleotide, and the second Cas protein and the second gRNA are encoded in a second polynucleotide.

318. A guide RNA (gRNA) that binds a target site in an enhancer region of a frataxin (FXN) locus, wherein the target site is located within the genomic coordinates human genome assembly GRCh38 (hg38) chr9:69,027,282-69,028,497.

319. The gRNA of embodiment 318, wherein the target site is located within the genomic coordinates hg38 chr9:69,027,615-69,028,101.

320. The gRNA of any of embodiments 318 or 319, wherein the target site comprises the sequence set forth in SEQ ID NO:21, a contiguous portion thereof of at least 14 nt, or a complementary sequence of any of the foregoing.

321. The gRNA of any of embodiments 318-320, wherein the gRNA binds the sequence complementary to SEQ ID NO:21.

322. The gRNA of any of embodiments 318-321, wherein the gRNA comprises a gRNA spacer sequence comprising the sequence set forth in SEQ ID NO:42, or a contiguous portion thereof of at least 14 nt.

323. The gRNA of any of embodiments 318-322, wherein the gRNA further comprises the sequence set forth in SEQ ID NO:44.

324. The gRNA of any of embodiments 318-323, wherein the gRNA comprises the sequence set forth in SEQ ID NO:67, optionally wherein the gRNA is set forth in SEQ ID NO:67.

325. A guide RNA (gRNA) that binds a target site in a regulatory DNA element of a frataxin (FXN) locus wherein the target site comprises a sequence selected from any of SEQ ID NOS:1-10, a contiguous portion thereof of at least 14 nt, or a complementary sequence of any of the foregoing.

326. The gRNA of embodiment 325, wherein the gRNA binds the sequence complementary to any of SEQ ID NOS: 1-10.

327. The gRNA of embodiments 325 or 326, wherein the gRNA comprises a gRNA spacer sequence comprising a sequence selected from any of SEQ ID NOS:22-31, or a contiguous portion thereof of at least 14 nt.

328. The gRNA of any of embodiments 325-327, wherein the gRNA comprises a gRNA spacer sequence comprising SEQ ID NO:22, or a contiguous portion thereof of at least 14 nt.

329. The gRNA of any of embodiments 325-327, wherein the gRNA comprises a gRNA spacer sequence comprising SEQ ID NO:23, or a contiguous portion thereof of at least 14 nt.

330. The gRNA of any of embodiments 325-327, wherein the gRNA comprises a gRNA spacer sequence comprising SEQ ID NO:24, or a contiguous portion thereof of at least 14 nt.

331. The gRNA of any of embodiments 325-327, wherein the gRNA comprises a gRNA spacer sequence comprising SEQ ID NO:25, or a contiguous portion thereof of at least 14 nt.

332. The gRNA of any of embodiments 325-327, wherein the gRNA comprises a gRNA spacer sequence comprising SEQ ID NO:26, or a contiguous portion thereof of at least 14 nt.

333. The gRNA of any of embodiments 325-327, wherein the gRNA comprises a gRNA spacer sequence comprising SEQ ID NO:27, or a contiguous portion thereof of at least 14 nt.

334. The gRNA of any of embodiments 325-327, wherein the gRNA comprises a gRNA spacer sequence comprising SEQ ID NO:28, or a contiguous portion thereof of at least 14 nt.

335. The gRNA of any of embodiments 325-327, wherein the gRNA comprises a gRNA spacer sequence comprising SEQ ID NO:29, or a contiguous portion thereof of at least 14 nt.

336. The gRNA of any of embodiments 325-327, wherein the gRNA comprises a gRNA spacer sequence comprising SEQ ID NO:30, or a contiguous portion thereof of at least 14 nt.

337. The gRNA of any of embodiments 325-327, wherein the gRNA comprises a gRNA spacer sequence comprising SEQ ID NO:31, or a contiguous portion thereof of at least 14 nt.

338. The gRNA of any of embodiments 325-337, wherein the gRNA further comprises the sequence set forth in SEQ ID NO:44.

339. The gRNA of any of embodiments 325-338, wherein the gRNA comprises a sequence selected from any of SEQ ID NOS:47-56, optionally wherein the gRNA is set forth in any of SEQ ID NOS:47-56, optionally wherein the gRNA is set forth in SEQ ID NO:47 or 53.

340. A guide RNA (gRNA) that binds a target site in a regulatory DNA element of a frataxin (FXN) locus wherein the target site comprises a sequence selected from any of SEQ ID NOS: 11-20, a contiguous portion thereof of at least 14 nt, or a complementary sequence of any of the foregoing.

341. The gRNA of embodiment 340, wherein the gRNA binds the sequence complementary to any of SEQ ID NOS: 11-20.

342. The gRNA of embodiment 340 or 341, wherein the gRNA comprises a gRNA spacer sequence comprising a sequence selected from any of SEQ ID NOS:32-41, or a contiguous portion thereof of at least 14 nt.

343. The gRNA of any of embodiments 340-342, wherein the target site comprises a sequence selected from any of SEQ ID NOS: 12-14 and 16-19, a contiguous portion thereof of at least 14 nt, or a complementary sequence of any of the foregoing.

344. The gRNA of any of embodiments 340-343, wherein the gRNA comprises a gRNA spacer sequence comprising a sequence selected from any of SEQ ID NOS:33-35 and 37-40, or a contiguous portion thereof of at least 14 nt.

345. The gRNA of any of embodiments 340-344, wherein the gRNA further comprises the sequence set forth in SEQ ID NO:46.

346. The gRNA of any of embodiments 340-345, wherein the gRNA comprises a sequence selected from any of SEQ ID NOS:57-66, optionally wherein the gRNA is set forth in any of SEQ ID NOS:57-66.

347. The gRNA of any of embodiments 118-346, wherein the gRNA spacer sequence is between 14 nt and 24 nt, or between 16 nt and 22 nt in length.

348. The gRNA of any of embodiments 318-347, wherein the gRNA spacer sequence is 18 nt, 19 nt, 20 nt, 21 nt or 22 nt in length.

349. The gRNA of any of embodiments 318-348, wherein the gRNA comprises modified nucleotides for increased stability.

350. The gRNA of any of embodiments 318-349, wherein the gRNA is capable of complexing with the Cas protein or variant thereof.

351. The gRNA of any of embodiments 318-150, wherein the gRNA comprises a gRNA spacer sequence that is capable of hybridizing to the target site or is complementary to the target site.

352. A combination, comprising a first gRNA comprising the gRNA of any of embodiments 318-351, and one or more second gRNAs that binds to a second target site in a regulatory DNA element of a frataxin (FXN) locus.

353. The combination of embodiment 152, wherein the second gRNA comprises the gRNA of any of embodiments 318-351.

354. A combination, comprising:
a first gRNA that binds a first target site in an enhancer region of a frataxin (FXN) locus, wherein the first target site is located within the genomic coordinates human genome assembly GRCh38 (hg38) chr9:69,027,282-69,028,497; and
a second gRNA that binds a second target site in a promoter region of a FXN locus, wherein the second target site is located within the genomic coordinates hg38 chr9:68,940,179-69,205,519.

355. The combination of embodiment 354, wherein the first gRNA comprises a gRNA spacer sequence set forth in SEQ ID NO:42 or a contiguous portion thereof of at least 14 nt.

356. The combination of embodiment 354 or 355, wherein the second gRNA comprises a gRNA spacer sequence set forth in SEQ ID NO:22 or 28 or a contiguous portion thereof of at least 14 nt.

357. The combination of any of embodiments 354-356, wherein:
the first gRNA comprises a gRNA spacer sequence set forth in SEQ ID NO:42 or a contiguous portion thereof of at least 14 nt; and
the second gRNA comprises a gRNA spacer sequence set forth in SEQ ID NO:22 or a contiguous portion thereof of at least 14 nt.

358. The combination of any of embodiments 354-356, wherein:
the first gRNA comprises a gRNA spacer sequence set forth in SEQ ID NO:42 or a contiguous portion thereof of at least 14 nt; and
the second gRNA comprises a gRNA spacer sequence set forth in SEQ ID NO:28 or a contiguous portion thereof of at least 14 nt.

359. A combination, comprising:
a first gRNA that binds a first target site in a promoter region of a frataxin (FXN) locus, wherein the first target site is located within the genomic coordinates hg38 chr9:68,940,179-69,205,519; and
a second gRNA that binds a second target site in a promoter region of a FXN locus, wherein the second target site is located within the genomic coordinates hg38 chr9:68,940,179-69,205,519.

360. The combination of embodiment 359, wherein the combination comprises:
the first gRNA comprises a gRNA spacer sequence set forth in SEQ ID NO:22 or a contiguous portion thereof of at least 14 nt; and
the second gRNA comprises a gRNA spacer sequence set forth in SEQ ID NO:28 or a contiguous portion thereof of at least 14 nt.

361. A fusion protein comprising (1) a DNA-targeting domain or a component thereof and (2) at least one effector domain, wherein:
the DNA-targeting domain or a component thereof binds to a target site in a regulatory DNA element of a frataxin (FXN) locus; and
the effector domain increases transcription of the FXN locus.

362. The fusion protein of embodiment 361, wherein the DNA-targeting domain comprises a Clustered Regularly Interspaced Short Palindromic Repeats associated (Cas)-guide RNA (gRNA) combination comprising (a) a Cas protein or a variant thereof and (b) at least one gRNA; a zinc finger protein (ZFP); a transcription activator-like effector (TALE); a meganuclease; a homing endonuclease; or an I-SceI enzyme or a variant thereof, optionally wherein the DNA-targeting domain comprises a catalytically inactive variant of any of the foregoing.

363. The fusion protein of embodiment 361 or 362, wherein the DNA-targeting domain comprises a Cas-gRNA combination comprising a Cas protein or a variant thereof and at least one gRNA, and the component of the DNA-targeting domain is a Cas protein or a variant thereof.

364. The fusion protein of embodiment 362 or 363, wherein the gRNA binds to a target site in a regulatory DNA element of a frataxin (FXN) locus.

365. A fusion protein comprising (1) a Cas protein or a variant thereof and (2) at least one effector domain, wherein the effector domain increases transcription of the FXN locus.

366. A fusion protein comprising (1) a first polypeptide of a split variant Cas protein comprising an N-terminal fragment of a Cas protein and an N-terminal Intein, and (2) at least one effector domain, wherein the effector domain increases transcription of the FXN locus.

367. The fusion protein of embodiment 366, wherein the first polypeptide of the split variant Cas protein, and a second polypeptide of the split variant Cas protein comprising a C-terminal fragment of the variant Cas protein and a C-terminal Intein, are present in proximity or present in the same cell, the N-terminal Intein and C-terminal Intein self-excise and ligate the N-terminal fragment and the C-terminal fragment of the variant Cas9 to form a full-length variant Cas9 protein.

368. A fusion protein comprising (1) a second polypeptide of a split variant Cas protein comprising a C-terminal fragment of a Cas protein and a C-terminal Intein and (2) at least one effector domain, wherein the effector domain increases transcription of the FXN locus.

369. The fusion protein of embodiment 368, wherein the second polypeptide of the split variant Cas protein, and a first polypeptide of the split variant Cas protein comprising an N-terminal fragment of the variant Cas protein and an N-terminal Intein, are present in proximity or present in the same cell, the N-terminal Intein and C-terminal Intein self-excise and ligate the N-terminal fragment and the C-terminal fragment of the variant Cas9 to form a full-length variant Cas9 protein.

370. The fusion protein of any of embodiments 362-369, wherein the Cas protein or a variant thereof is capable of complexing with at least one gRNA, optionally wherein the gRNA binds to a target site in a regulatory DNA element of a frataxin (FXN) locus.

371. The fusion protein of embodiment 361, wherein binding of the DNA-targeting domain or a component thereof to the target site does not introduce a genetic disruption or a DNA break at or near the target site.

372. The fusion protein of any of embodiments 362-371, wherein the Cas protein or a variant thereof is a deactivated Cas (dCas) protein.

373. The fusion protein of any of embodiments 362-372, wherein the Cas protein or a variant thereof is a Cas9 protein or a variant thereof.

374. The fusion protein of any of embodiments 362-373, wherein the Cas protein or a variant thereof is a deactivated Cas9 (dCas9) protein.

375. The fusion protein of any of embodiments 362-374, wherein the Cas9 protein or a variant thereof is a Staphylococcus aureus Cas9 (SaCas9) protein or a variant thereof.

376. A fusion protein comprising (1) a Staphylococcus aureus dCas9 protein (dSaCas9) and (2) at least one effector domain, wherein the effector domain increases transcription of a frataxin (FXN) locus.

377. The fusion protein of any of embodiments 362-376, wherein the Cas9 protein or a variant thereof is a Staphylococcus aureus dCas9 protein (dSaCas9) that comprises at least one amino acid mutation selected from D10A and N580A, with reference to numbering of positions of SEQ ID NO:73.

378. The fusion protein of any of embodiments 362-377, wherein the Cas9 protein or a variant thereof comprises the sequence set forth in SEQ ID NO:72, or an amino acid sequence that has at least 90%, 91%, 92%, 93%, 94%, 95%, 96%, 97%, 98%, or 99% sequence identity thereto.

379. The fusion protein of any of embodiments 362-374, wherein the Cas9 protein or variant thereof is a Streptococcus pyogenes Cas9 (SpCas9) protein or a variant thereof.

380. The fusion protein of any of embodiments 362-374 and 379, wherein the Cas9 protein or a variant thereof is a Streptococcus pyogenes dCas9 (dSpCas9) protein that comprises at least one amino acid mutation selected from D10A and H840A, with reference to numbering of positions of SEQ ID NO:79.

381. The fusion protein of any of embodiments 362-374, 379 and 380, wherein the Cas9 protein or a variant thereof comprises the sequence set forth in SEQ ID NO:78, or an amino acid sequence that has at least 90%, 91%, 92%, 93%, 94%, 95%, 96%, 97%, 98%, or 99% sequence identity thereto.

382. The fusion protein of any of embodiments 362-381, wherein the Cas protein or variant thereof is a split variant Cas protein, wherein the split variant Cas protein comprises a first polypeptide comprising an N-terminal fragment of the variant Cas protein and an N-terminal Intein, and a second polypeptide comprising a C-terminal fragment of the variant Cas protein and a C-terminal Intein.

383. The fusion protein of embodiment 382, wherein when the first polypeptide and the second polypeptide of the split variant Cas protein are present in proximity or present in the same cell, the N-terminal Intein and C-terminal Intein self-excise and ligate the N-terminal fragment and the C-terminal fragment of the variant Cas protein to form a full-length variant Cas protein.

384. The fusion protein of embodiment 382 or 383, wherein the N-terminal Intein comprises an N-terminal Npu Intein, or the sequence set forth in SEQ ID NO:178, or an amino acid sequence that has at least 90%, 91%, 92%, 93%, 94%, 95%, 96%, 97%, 98%, or 99% sequence identity thereto, or a portion of any of the foregoing.

385. The fusion protein of any of embodiments 382-384, wherein N-terminal fragment of the variant Cas protein comprises:
the N-terminal fragment of variant SpCas9 from the N-terminal end up to position 573 of the dSpCas9 sequence set forth in SEQ ID NO:78, or an amino acid sequence that has at least 90%, 91%, 92%, 93%, 94%, 95%, 96%, 97%, 98%, or 99% sequence identity thereto; or
the sequence set forth in SEQ ID NO: 176, or an amino acid sequence that has at least 90%, 91%, 92%, 93%, 94%, 95%, 96%, 97%, 98%, or 99% sequence identity thereto, or a portion of any of the foregoing.

386. The fusion protein of any of embodiments 382-385, wherein the C-terminal Intein comprises a C-terminal Npu Intein, or the sequence set forth in SEQ ID NO: 182, or an amino acid sequence that has at least 90%, 91%, 92%, 93%, 94%, 95%, 96%, 97%, 98%, or 99% sequence identity thereto, or a portion of any of the foregoing.

387. The fusion protein of any of embodiments 382-386, wherein C-terminal fragment of the variant Cas protein comprises:
the C-terminal fragment of variant SpCas9 from position 574 to the C-terminal end of the dSpCas9 sequence set forth in SEQ ID NO:78, or an amino acid sequence that has at least 90%, 91%, 92%, 93%, 94%, 95%, 96%, 97%, 98%, or 99% sequence identity thereto; or
the sequence set forth in SEQ ID NO: 184, or an amino acid sequence that has at least 90%, 91%, 92%, 93%, 94%, 95%, 96%, 97%, 98%, or 99% sequence identity thereto, or a portion of any of the foregoing.

388. The fusion protein of any of embodiments 361-387, wherein the regulatory DNA element is an enhancer.

389. The fusion protein of any of embodiments 361-388, wherein the target site is located within the genomic coordinates human genome assembly GRCh38 (hg38) chr9:69,027,282-69,028,497.

390. The fusion protein of any of embodiments 361-389, wherein the target site is located within the genomic coordinates hg38 chr9:69,027,615-69,028, 101.

391. The fusion protein of any of embodiments 361-390, wherein the target site comprises the sequence set forth in SEQ ID NO:21, a contiguous portion thereof of at least 14 nt, or a complementary sequence of any of the foregoing.

392. The fusion protein of any of embodiments 361-387, wherein the regulatory DNA element is a promoter.

393. The fusion protein of any of embodiments 361-387 and 392, wherein the target site is located within the genomic coordinates hg38 chr9:68,940,179-69,205,519.

394. The fusion protein of any of embodiments 361-387, 392, and 393, wherein the target site comprises a sequence selected from any of SEQ ID NOS: 1-10, a contiguous portion thereof of at least 14 nt, or a complementary sequence of any of the foregoing.

395. The fusion protein of any of embodiments 361-387, and 392-394, wherein the target site comprises a sequence selected from any of SEQ ID NOS: 11-20, a contiguous portion thereof of at least 14 nt, or a complementary sequence of any of the foregoing.

396. The fusion protein of any of embodiments 361-395, wherein the effector domain induces transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation.

397. The fusion protein of any of embodiments 361-396, wherein the effector domain induces transcription activation, transcription co-activation, transcription elongation.

398. The fusion protein of any of embodiments 361-397, wherein the effector domain induces transcription activation.

399. The fusion protein of any of embodiments 361-398, wherein the effector domain comprises at least one VP16 domain, or a VP16 tetramer (“VP64”) or a variant thereof.

200. The fusion protein of any of embodiments 361-399, wherein the effector domain comprises the sequence set forth in SEQ ID NO:81 or 83, or a portion thereof, or an amino acid sequence that has at least 90%, 91%, 92%, 93%, 94%, 95%, 96%, 97%, 98%, or 99% sequence identity to any of the foregoing.

401. The fusion protein of any of embodiments 361-398, wherein the effector domain is selected from a p65 activation domain, a p300 domain, DPOLA, ENL, FOXO3, HSH2D, NCOA2, NCOA3, PSA1, PYGO1, RBM39, HERC2, DMD, or NOTCH2, or a domain thereof, a portion thereof or a variant thereof.

402. The fusion protein of any of embodiments 361-398 and 401, wherein the effector domain comprises a sequence selected from any of SEQ ID NOS: 113-125, or a domain thereof, a portion thereof, or an amino acid sequence that has at least 90%, 91%, 92%, 93%, 94%, 95%, 96%, 97%, 98%, or 99% sequence identity to any of the foregoing.

403. The fusion protein of any of embodiments 361-398, 401, and 402, wherein the effector domain comprises a sequence selected from any of SEQ ID NOS: 100-112, or a domain thereof, a portion thereof, or an amino acid sequence that has at least 90%, 91%, 92%, 93%, 94%, 95%, 96%, 97%, 98%, or 99% sequence identity to any of the foregoing.

404. The fusion protein of any of embodiments 399-403, wherein the variant thereof comprises a truncations thereof.

405. The fusion protein of any of embodiments 361-404, wherein the at least one effector domain is fused to the N-terminus, the C-terminus, or both the N-terminus and the C-terminus, of the DNA-targeting domain or a component thereof, optionally wherein the at least one effector domain is fused to the N-terminus, the C-terminus, or both the N-terminus and the C-terminus of the Cas protein or a variant thereof.

406. The fusion protein of any of embodiments 361-405, further comprising one or more linkers connecting the DNA-targeting domain or a component thereof, optionally the Cas protein or variant thereof, to the at least one effector domain, and/or further comprising one or more nuclear localization signals (NLS).

407. The fusion protein of any of embodiments 361-400, 405, and 406, wherein the fusion protein comprises the sequence set forth in SEQ ID NO:71, or an amino acid sequence that has at least 90%, 91%, 92%, 93%, 94%, 95%, 96%, 97%, 98%, or 99% sequence identity thereto.

408. The fusion protein of any of embodiments 361-400, 405, and 406, wherein the fusion protein comprises the sequence set forth in SEQ ID NO:77, or an amino acid sequence that has at least 90%, 91%, 92%, 93%, 94%, 95%, 96%, 97%, 98%, or 99% sequence identity thereto.

409. A combination comprising the fusion protein of any of embodiments 361-408 and at least one gRNA, optionally wherein the at least one gRNA is a gRNA of any of embodiments 318-351.

410. A polynucleotide encoding the DNA-targeting system of any of embodiments 201-281, the combination of any of embodiments 282-317, 352-360, and 409, the gRNA of any of embodiments 318-351, or the fusion protein of any of embodiments 361-408, or a portion or a component of any of the foregoing.

411. A polynucleotide encoding a first DNA-targeting system, a first Cas protein and/or a first gRNA of the combination of any of embodiments 282-317, 352-360, and 409.

412. A polynucleotide encoding a second DNA-targeting system, a second Cas protein and/or a second gRNA of the DNA-targeting system of the combination of any of embodiments 282-317, 352-360, and 409.

413. A plurality of polynucleotides, comprising the polynucleotide of any of embodiments 410-412, and one or more additional polynucleotides encoding an additional portion or an additional component of the DNA-targeting system of any of embodiments 201-281, the combination of any of embodiments 282-317, 352-360, and 409, the gRNA of any of embodiments 318-351, or the fusion protein of any of embodiments 361-408, or a portion or a component of any of the foregoing.

414. A plurality of polynucleotides, comprising:
a first polynucleotide comprising the polynucleotide of embodiment 411; and
a second polynucleotide comprising the polynucleotide of embodiment 412.

415. A vector comprising the polynucleotide of any of embodiments 410-412, the plurality of polynucleotides of embodiment 413 or 414, or a first polynucleotide or a second polynucleotide of the plurality of polynucleotides of embodiment 413 or 414, or a portion or a component of any of the foregoing.

416. The vector of embodiment 415, wherein the vector is a viral vector, optionally wherein the viral vector is an AAV vector.

417. The vector of embodiment 416, wherein the viral vector, optionally the AAV vector, exhibits tropism for a cell of the central nervous system (CNS), a heart cell, optionally a cardiomyocyte, a skeletal muscle cell, a fibroblast, an induced pluripotent stem cell, or a cell derived from any of the foregoing.

418. The vector of embodiment 416 or 417, wherein the viral vector is an AAV vector and the AAV vector is selected from among AAV1, AAV2, AAV3, AAV4, AAV5, AAV6, AAV7, AAV8, AAV9, AAV10, AAV11, AAV12, or AAV-DJ vector, optionally an AAV5 vector or an AAV9 vector.

419. The vector of any of embodiments 416-418, wherein the viral vector is an AAV9 vector.

420. The vector of embodiment 415, wherein the vector is a non-viral vector selected from a lipid nanoparticle, a liposome, an exosome, or a cell penetrating peptide

421. A plurality of vectors, comprising the vector of any of embodiments 415-420, and one or more additional vectors comprising one or more additional polynucleotides encoding an additional portion or an additional component of the DNA-targeting system of any of embodiments 201-281, the combination of any of embodiments 282-317, 352-360, and 409, the gRNA of any of embodiments 318-351, or the fusion protein of any of embodiments 361-408, or a portion or a component of any of the foregoing.

422. A plurality of vectors, comprising:
a first vector comprising the polynucleotide of embodiment 410; and
a second vector comprising the polynucleotide of embodiment 411.

423. A cell comprising the DNA-targeting system of any of embodiments 201-281, the combination of any of embodiments 282-317, 352-360, and 409, the gRNA of any of embodiments 318-351, the fusion protein of any of embodiments 361-408, the polynucleotide of any of embodiments 410-412, the plurality of polynucleotides of embodiment 413 or 414, the vector of any of embodiments 415-420, the plurality of vectors of embodiment 421 or 422, or a portion or a component of any of the foregoing.

424. The cell of embodiment 423, wherein the cell is a heart cell, a skeletal muscle cell, a nervous system cell, or an induced pluripotent stem cell.

425. The cell of embodiment 423 or 424, wherein the cell is from a subject that has or is suspected of having Friedreich's ataxia (FA).

426. A method for modulating the expression of frataxin (FXN) in a cell, the method comprising:
introducing the DNA-targeting system of any of embodiments 201-281, the combination of any of embodiments 282-317, 352-360, and 409, the gRNA of any of embodiments 318-351, the fusion protein of any of embodiments 361-408, the polynucleotide of any of embodiments 410-412, the plurality of polynucleotides of embodiment 413 or 414, the vector of any of embodiments 415-420, the plurality of vectors of embodiment 421 or 422, or a portion or a component of any of the foregoing, into the cell.

427. A method of inducing a genetic disruption at a target site in an enhancer region of a frataxin (FXN) locus in a cell, wherein the target site is located within the genomic coordinates human genome assembly GRCh38 (hg38) chr9:69,027,282-69,028,497, the method comprising:
contacting a cell with the DNA-targeting system of the DNA-targeting system of any of embodiments 201-281, the combination of any of embodiments 282-317, 352-360, and 409, the gRNA of any of embodiments 318-351, the fusion protein of any of embodiments 361-408, the polynucleotide of any of embodiments 410-412, the plurality of polynucleotides of embodiment 413 or 414, the vector of any of embodiments 415-420, the plurality of vectors of embodiment 421 or 422, the plurality of vectors of embodiment 164 or 165, or a portion or a component of any of the foregoing.

428. The method of embodiment 426 or 427, wherein the cell is from a subject that has or is suspected of having Friedreich's ataxia (FA).

429. A method for modulating the expression of frataxin (FXN) in a subject, the method comprising:
administering the DNA-targeting system of any of embodiments 201-281, the combination of any of embodiments 282-317, 352-360, and 409, the gRNA of any of embodiments 318-351, the fusion protein of any of embodiments 361-408, the polynucleotide of any of embodiments 410-412, the plurality of polynucleotides of embodiment 413 or 414, the vector of any of embodiments 415-420, the plurality of vectors of embodiment 421 or 422, or a portion or a component of any of the foregoing, to the subject.

430. The method of embodiment 428 or 429, wherein the subject has or is suspected of having Friedreich's ataxia (FA).

431. A method of treating Friedreich's ataxia (FA), the method comprising:
administering the DNA-targeting system of any of embodiments 201-281, the combination of any of embodiments 282-317, 352-360, and 409, the gRNA of any of embodiments 318-351, the fusion protein of any of embodiments 361-408, the polynucleotide of any of embodiments 410-412, the plurality of polynucleotides of embodiment 413 or 414, the vector of any of embodiments 415-420, the plurality of vectors of embodiment 421 or 422, or a portion or a component of any of the foregoing, to a subject that has or is suspected of having FA.

432. The method of any of embodiments 428-431, wherein a cell in the subject exhibits reduced expression of FXN compared to a cell from a normal subject.

433. The method of any of embodiments 428-432, wherein a cell in the subject has a GAA trinucleotide repeat expansion in the FXN gene.

434. The method of any of embodiments 426-433, wherein the cell is a heart cell, a skeletal muscle cell, a nervous system cell, or an induced pluripotent stem cell.

435. The method of any of embodiments 426-434, wherein the introducing, contacting or administering is carried out in vivo or ex vivo.

436. The method of any of embodiments 426-435, wherein following the introducing, contacting or administering, the expression of frataxin (FXN) is increased in the cell or the subject.

437. The method of embodiment 436, wherein the expression is increased at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold.

438. The method of embodiment 436 or 437, wherein the expression is increased by less than about 10-fold, 9-fold, 8-fold, 7-fold or 6-fold.

439. The method of any of embodiments 428-438, wherein the subject is a human.

440. A pharmaceutical composition comprising the DNA-targeting system of any of embodiments 201-481, the combination of any of embodiments 282-317, 352-360, and 409, the gRNA of any of embodiments 318-351, the fusion protein of any of embodiments 361-408, the polynucleotide of any of embodiments 410-412, the plurality of polynucleotides of embodiment 413 or 414, the vector of any of embodiments 415-420, the plurality of vectors of embodiment 421 or 422, or a portion or a component of any of the foregoing.

441. The pharmaceutical composition of embodiment 440, for use in treating Friedreich's ataxia (FA).

442. The pharmaceutical composition of embodiment 440, for use in the manufacture of a medicament for treating Friedreich's ataxia (FA).

443. The pharmaceutical composition for use of embodiment 441 or 442, wherein the pharmaceutical composition is to be administered to a subject.

444. Use of the pharmaceutical composition of embodiment 440 for treating Friedreich's ataxia (FA).

445. Use of the pharmaceutical composition of embodiment 440 in the manufacture of a medicament for treating Friedreich's ataxia (FA).

446. The use of embodiment 444 or 445, wherein the pharmaceutical composition is to be administered to a subject.

447. The pharmaceutical composition for use or the use of any of embodiments 441-446, wherein the subject has or is suspected of having Friedreich's ataxia (FA).

448. The pharmaceutical composition for use or the use of any of embodiments 441-447, wherein a cell in the subject exhibits reduced expression of FXN compared to a cell from a normal subject.

449. The pharmaceutical composition for use or the use of any of embodiments 441-448, wherein a cell in the subject has a GAA trinucleotide repeat expansion in the FXN gene.

450. The pharmaceutical composition for use or the use of embodiment 448 or 449, wherein the cell is a heart cell, a skeletal muscle cell, a nervous system cell, or an induced pluripotent stem cell.

451. The pharmaceutical composition for use or the use of any of embodiments 443 and 446-450, wherein the administration is carried out in vivo or ex vivo.

452. The pharmaceutical composition for use or the use of any of embodiments 443 and 446-451, wherein following the administration, the expression of frataxin (FXN) is increased in the cell or the subject.

453. The pharmaceutical composition for use or the use of embodiment 452, wherein the expression is increased at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold.

454. The pharmaceutical composition for use or the use of embodiment 452 or 453, wherein the expression is increased by less than about 10-fold, 9-fold, 8-fold, 7-fold or 6-fold.

455. The pharmaceutical composition for use or the use of any of embodiments 443 and 446-454, wherein the subject is a human.

### VIII. Examples

The following examples are included for illustrative purposes only and are not intended to limit the scope of the invention.

### Example 1: CRISPR-Cas Mediated Transcriptional Activation of Frataxin (FXN) in Induced Pluripotent Stem Cells (iPSCs)

Guide RNAs (gRNAs) targeting the human frataxin (FXN) gene promoter were generated and transduced with deactivated Cas9 (dCas9)-transcriptional activator fusion proteins into wild-type (WT) and Friedreich's ataxia (FA) patient-derived cells and frataxin mRNA expression was assessed.

A. dSpCas9-2× VP64, dSaCas9-2× VP64 and gRNA Constructs

Plasmids encoding the heterologous fusion proteins dSpCas9-2× VP64 or dSaCas9-2× VP64 were prepared. Fusion proteins included a modified Cas9 engineered to lack endonuclease activity (deactivated Cas9, dCas9) from S. aureus (dSaCas9) or S. pyogenes (dSpCas9), fused with 2 copies of the transcriptional activator VP64 (2× VP64), one copy at each of the N- and C-terminals.

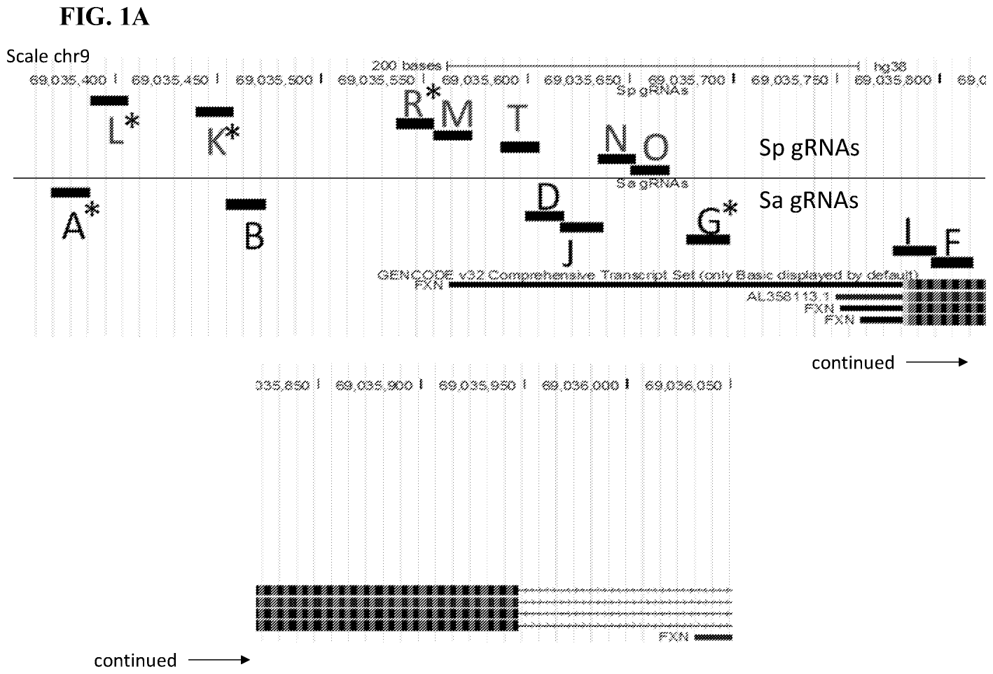

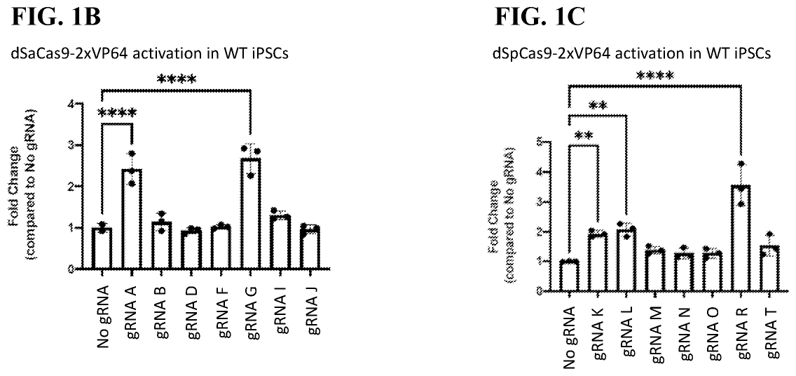

Plasmids were prepared, each encoding a gRNA targeting one of multiple sequences in the human frataxin gene promoter region (Homo sapiens (human) genome assembly GRCh38 (hg38) chr9:69034622-69036670), as shown in FIG. 1A. The gRNAs included a 20-22 nucleotide (nt) DNA targeting spacer sequence and a constant scaffold sequence set forth in SEQ ID NO:22 and SEQ ID NO:23. Different gRNAs were designed for dSaCas9-2× VP64 and dSpCas9-2× VP64, based on the protospacer-adjacent motif (PAM) sequences 5′-NNGRRT-3′ or 5′-NGG-3′, respectively. The promoter-targeting gRNAs are indicated in Table E1.

TABLE E1
Frataxin promoter-targeting gRNAs

| SEQ ID | gRNA | gRNA spacer sequence | dCas9 | |
|---|---|---|---|---|
| NO: | name | (DNA) | variant | PAM |
| 1 | gRNA A | TCACACAGCTAGGAAGTGGG | S. aureus | 5′-NNGRRT-3′ |
| 2 | gRNA B | GAGGCTGCTTGGCCGCCGGT | S. aureus | 5′-NNGRRT-3′ |
| 3 | gRNA C | GCCCGCTCCGCCCTCCAGCG | S. aureus | 5′-NNGRRT-3′ |
| 4 | gRNA D | AGCCTGCTTTGTGCAAAGCA | S. aureus | 5′-NNGRRT-3′ |
| 5 | gRNA E | CCGGCCGATGACGCGCCGCGGG | S. aureus | 5′-NNGRRT-3′ |
| 6 | gRNA F | GGCCGGCTACTGCGCGGCGCCC | S. aureus | 5′-NNGRRT-3′ |
| 7 | gRNA G | ACCTCTAGCTGCTCCCCCACAG | S. aureus | 5′-NNGRRT-3′ |
| 8 | gRNA H | CCGCCCGCTCCGCCCTCCAGCG | S. aureus | 5′-NNGRRT-3′ |
| 9 | gRNA I | GCCCGAGAGTCCACATGCTGCT | S. aureus | 5′-NNGRRT-3′ |
| 10 | gRNA J | GCTCTCCATTTTTGTTAAATGC | S. aureus | 5′-NNGRRT-3′ |
| 11 | gRNA K | CTGCTGTAAACCCATACCGG | S. pyogenes | 5′-NGG-3′ |
| 12 | gRNA L | GCAGAGTACAGATTTACACA | S. pyogenes | 5′-NGG-3′ |
| 13 | gRNA M | CAAGGGAGACTGCAGCCTGG | S. pyogenes | 5′-NGG-3′ |
| 14 | gRNA N | AAGCTGGGAAGTTCTTCCTG | S. pyogenes | 5′-NGG-3′ |
| 15 | gRNA O | ATGCACGAATAGTGCTAAGC | S. pyogenes | 5′-NGG-3′ |
| 16 | gRNA P | TACACAAGGCATCCGTCTCC | S. pyogenes | 5′-NGG-3′ |
| 17 | gRNA Q | GGCTGCTTGGCCGCCGGTAT | S. pyogenes | 5′-NGG-3′ |
| 18 | gRNA R | TTTAGAAGCGGCGGGCCACC | S. pyogenes | 5′-NGG-3′ |
| 19 | gRNA S | AATTGAGGCTGCTTGGCCGC | S. pyogenes | 5′-NGG-3′ |
| 20 | gRNA T | GCAAAGCACGGAGTGCAACC | S. pyogenes | 5′-NGG-3′ |

B. Delivery of dCas9-2× VP64 and gRNAs to iPSCs

Wild-type induced pluripotent stem cells (iPSCs) cultured on Matrigel (Corning) with mTeSR Plus medium (Stemcell Technologies) were transduced using lentivirus with a plasmid encoding one of the frataxin promoter-targeting gRNAs described in Table E1 above and corresponding dSaCas9-2× VP64 or dSpCas9-2× VP64. Cells were incubated for 2 days, then cultured in the presence of 1 μg/mL puromycin to enrich for transduced cells for an additional 2 days. Cells were then split using Accutase cell dissociation reagent (ThermoFisher #A1110501) and cultured for an additional 3 days prior to assessing frataxin expression levels.

C. Assessing Frataxin Expression in iPSCs Cultured with dCas9-2× VP64 and gRNA

Frataxin mRNA expression levels were assessed by RT-qPCR. Approximately 30,000-60,000 transduced iPSCs per condition were harvested and assayed by RT-qPCR using a Cells-to-Cr kit (ThermoFisher) to assess relative changes in expression of frataxin mRNA. For each condition, frataxin (FXN) expression was normalized to GAPDH expression. Taqman probes were used for FXN (ThermoFisher, Cat #: Hs00175940_m1) and GAPDH (ThermoFisher, Cat #: Hs02786624_g1). Results were assessed as fold-increase expression of FXN relative to control cells that were not transduced with gRNA.

Frataxin promoter-targeted gRNA A and gRNA G with dSaCas9-2× VP64 induced a greater than 2-fold increase in frataxin mRNA expression, compared to controls without gRNA (FIG. 1B). Similar results were obtained for gRNA K, gRNA L, and gRNA R with dSpCas9-2× VP64 (FIG. 1C).

The results indicate that dCas9-effector fusion protein with gRNA targeted to the frataxin promoter can increase frataxin mRNA expression levels in human iPSCs.

D. Transcriptional Activation of Frataxin in Friedreich's Ataxia Patient-Derived iPSCs (FA-iPSCs) Harboring Large Repeat Expansions

Friedreich's ataxia (FA) is associated with GAA trinucleotide expansion mutations in the first intron of the frataxin gene. Trinucleotide expansion results in reduced frataxin expression, leading to disease. Increasing trinucleotide expansion lengths are associated with decreased frataxin expression levels, earlier disease onset, and increased disease severity.

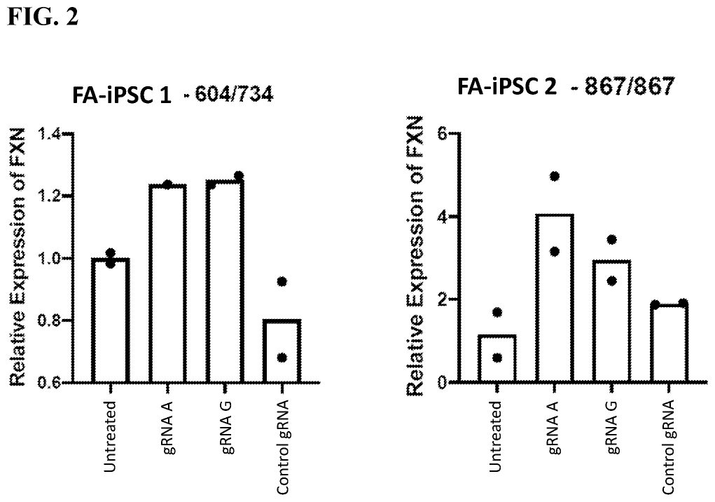

iPSCs generated from Friedreich's ataxia patients iPSCs (FA-iPSCs) containing different lengths of expanded GAA trinucleotide repeats (FA-iPSC 1 with 604/734 repeats; FA-iPSC 2 with 867/867 repeats) in the frataxin gene were (a) left untreated, (b) transduced with dSaCas9-2× VP64 and a control gRNA, or (c) transduced with dSaCas9-2× VP64 and promoter-targeting gRNA A and gRNA G. Frataxin mRNA expression was assessed by RT-qPCR as described above. FA-iPSCs transduced with the promoter-targeting gRNAs exhibited increased frataxin mRNA expression in comparison to FA-iPSCs that were untreated or transduced with control gRNA (FIG. 2). The results indicated that dCas9-transcriptional activator with gRNA targeted to the frataxin promoter can increase frataxin mRNA expression levels in the context of FA-iPSCs with various lengths of GAA trinucleotide expansion mutations. The results support the utility of frataxin-targeting gRNAs and dCas9-transcriptional activator fusion proteins in restoring frataxin expression levels in cells from FA patients and potential treatment of FA.

### Example 2: A Saturating Screen of gRNAs Targeting Frataxin for CRISPR-Based Transcriptional Activation

A saturating library of gRNAs was designed and generated for a broad genomic region including and surrounding the frataxin gene. gRNAs were tested in a pooled format in iPSCs expressing dCas9-transcriptional activator fusion proteins, to identify gRNAs that facilitate increased frataxin expression.

A. Design and Generation of Frataxin-Saturating gRNA Library

A saturating library of SaCas9 gRNAs was designed and generated, targeting a broad genomic region of approximately 250 kb including and surrounding the frataxin gene (genomic region: hg38 chr9:68,940,179-69,205.519). gRNAs were designed according to the PAM sequence for SaCas9 (5′-NNGRRT-3′). gRNAs were excluded if spacers contained 5 or more continuous T nucleotides to avoid premature termination of the gRNA transcripts. gRNAs with a high level of predicted off-target activity in human cells were also excluded. In total, 6173 gRNAs, including 300 control gRNAs, were designed, cloned, and tested for ability to transcriptionally activate the expression of frataxin, together with dSaCas9-2× VP64.

### B. Flow Cytometry Assay for Frataxin Protein Expression Levels

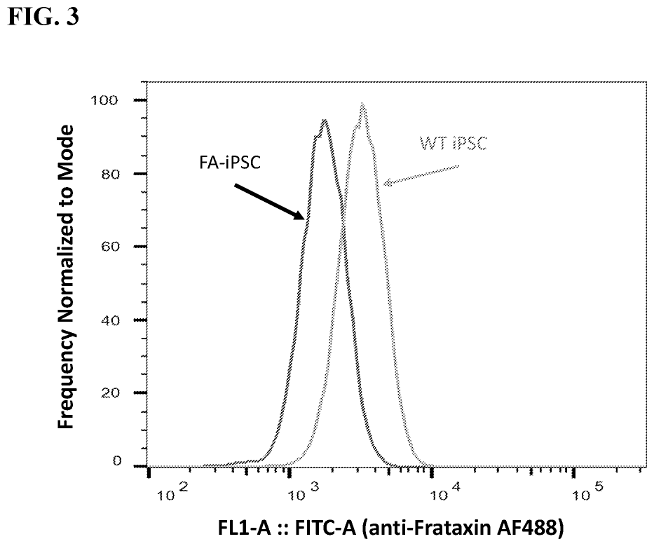

WT-iPSCs and long GAA trinucleotide repeat FA-iPSCs were assessed by flow cytometry for levels of frataxin protein expression. Cells were fixed with 4% paraformaldehyde (PFA) at room temperature, permeabilized with MeOH overnight at −20° C. and labeled with a mouse monoclonal anti-frataxin fluorophore-conjugated antibody (abcam ab156033). Cells were then assessed by flow cytometry for frataxin protein expression levels. The results showed that FA-iPSCs exhibited reduced frataxin expression in comparison to WT-iPSC control cells (FIG. 3).

C. Screen of Frataxin-Saturating gRNA Library for Transcriptional Activation of Frataxin

gRNAs with strong activity of increasing frataxin expression were identified from screening the saturating gRNA library. WT-iPSCs were transduced with a lentiviral construct containing dSaCas9-2× VP64 and cultured in the presence of puromycin to select for transduced cells. The WT-iPSCs expressing dSaCas9-2× VP64 were then transduced using lentivirus with the pooled gRNA library at a multiplicity of infection (MOI) of 0.4 (i.e. 0.4 viral particles per cell) and incubated for 1 day. Cells were then cultured for 7 days in the presence of 1 μg/mL puromycin to enrich for transduced cells. Cells were then fixed with 4% PFA at room temperature, permeabilized with MeOH overnight at −20° C. and labeled with a mouse monoclonal anti-frataxin fluorophore-conjugated antibody (abcam ab156033). Cells were sorted by flow cytometry into 2 populations, representing the top 10% and bottom 10% of cells based on frataxin protein expression.

Sequencing was performed to compare the abundance of specific gRNAs between the frataxin-low and frataxin-high population, and to identify gRNAs enriched in the frataxin-high population. Genomic DNA was isolated from the sorted populations. Targeted PCR was performed to amplify the gRNA spacers and append sequencing adapters. Each sample was barcoded separately. Samples were then sequenced using an Illumina MiSeq System. Three sequencing replicates of the frataxin-high population were compared to three replicates of the frataxin-low population using DEseq2, a method for detecting differentially expressed transcripts.

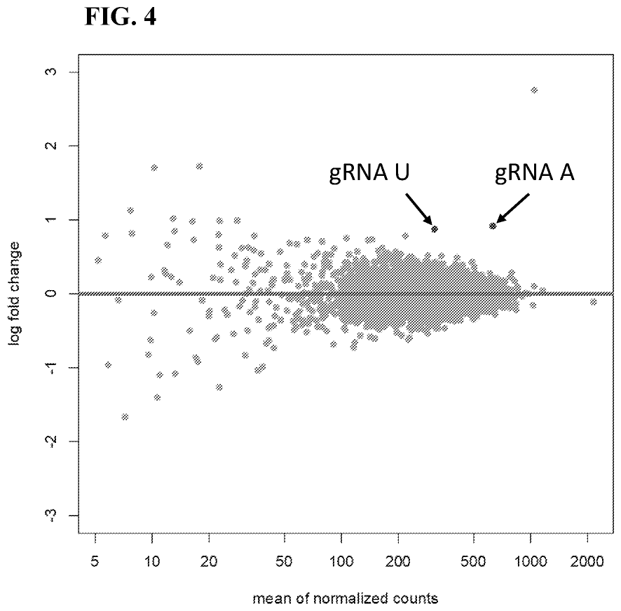

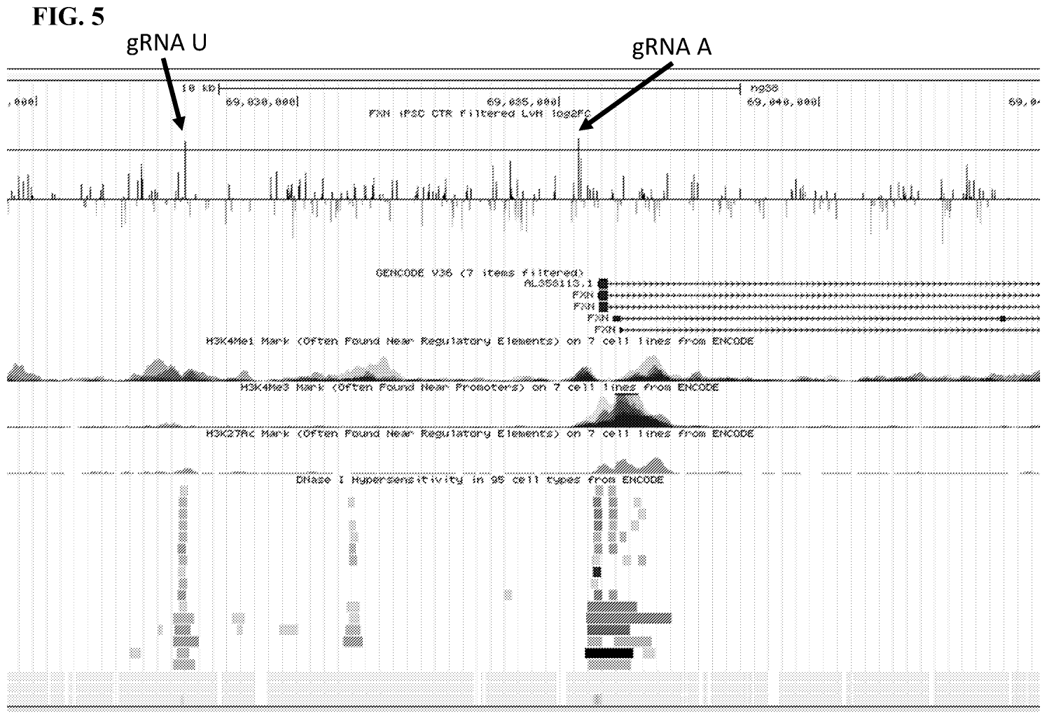

gRNAs enriched in the frataxin-high population were identified based on sequencing analysis (FIG. 4). gRNAs that were enriched in the frataxin-high population included the promoter-targeting gRNA A from Example 1 above, and a second gRNA (gRNA U; spacer sequence set forth in SEQ ID NO:21). gRNA U targeted a region approximately 7 kb upstream of the frataxin promoter. This region (hg38 chr9:69,027,282-69,028,497) exhibits hallmark chromatin signatures of an enhancer element, including specific histone modifications (H3K4Me1) and DNase I hypersensitivity (FIG. 5). Although similar enhancer-like signatures were present in the screened region (for example, hg38 chr9:69,044,201-69,045,347 and hg38 chr9: 69,030,752-69,031,507), gRNAs targeting these regions were not identified as significantly enriched in the frataxin-high population.

The results showed that a gRNA targeting an upstream enhancer region of the frataxin gene, together with a dCas9-transcriptional activator fusion protein, leads to a strong increase in frataxin expression, supporting the utility of the exemplary DNA-targeting system and gRNAs in restoring frataxin expression levels in FA patient cells and potential treatment of FA.

### Example 3: Combinatorial Modulation of Frataxin Expression

An exemplary frataxin enhancer region-targeting gRNA (gRNA U, described in Example 2C above) was tested alone and in combination with promoter-targeting gRNAs in iPSCs, for transcriptional activation of frataxin.

A. Enhancer Region-Targeting gRNA

WT-iPSCs were transduced with dSaCas9-2× VP64 and (a) gRNA A (promoter-targeting, see Example 1 above), (b) gRNA U (enhancer-targeting, see Example 2C above), (c) a control gRNA (not frataxin-targeting), or (d) an empty gRNA vector. Cells were incubated for 2 days, then cultured in the presence of 1 μg/mL puromycin to enrich for transduced cells for an additional 2 days. Cells were then split using Accutase cell dissociation reagent (ThermoFisher #A1110501) and cultured for an additional 3 days prior to assessing frataxin expression levels. Relative frataxin mRNA expression levels were assessed by RT-qPCR as described above.

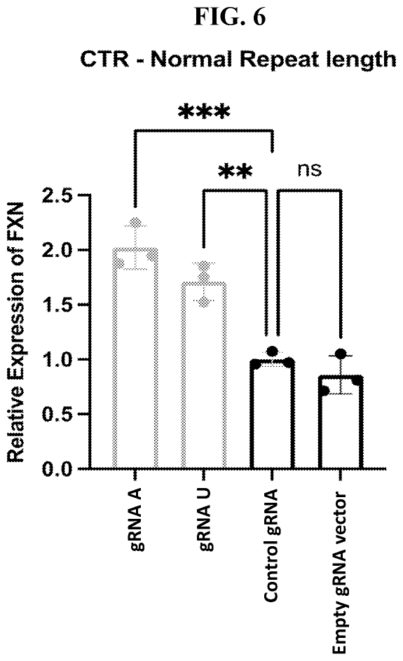

Cells transduced with gRNA A or gRNA U exhibited a greater than 1.5-fold increase in frataxin mRNA expression in comparison to cells transduced with the control gRNA or empty gRNA vector (FIG. 6).

B. iPSC Lines Stably Expressing Frataxin Promoter-Targeting gRNAs

WT-iPSCs or FA-iPSCs harboring short or long frataxin trinucleotide repeats were transduced with lentivirus containing expression vectors for frataxin promoter-targeting gRNAs A or G. Cells were then selected for transduced cells with blasticidin and stable cell lines were established expressing the promoter-targeting gRNAs, as indicated in Table E2.

TABLE E2
gRNA-expressing iPSC cell lines

| | | Stably |
|---|---|---|
| | | transduced |
| Name | Transduced cells | gRNA |
| WT-gRNA A-iPSC | WT-iPSCs | gRNA A |
| WT-gRNA G-iPSC | WT-IPSCs | gRNA G |
| FA(short)-gRNA | FA-iPSCs; short | gRNA A |
| A-iPSC | trinucleotide repeat | |
| FA(short)-gRNA | FA-iPSCs; short | gRNA G |
| G-iPSC | trinucleotide repeat | |
| FA(long)-gRNA | FA-iPSCs; long | gRNA A |
| A-iPSC | trinucleotide repeat | |
| FA(long)-gRNA | FA-iPSCs; long | gRNA G |
| G-iPSC | trinucleotide repeat | |

C. Combinatorial Modulation of Frataxin mRNA Expression with Promoter- and Enhancer-Targeting gRNAs

The enhancer-targeting gRNA U was assessed for activation of frataxin expression when paired with promoter-targeting gRNAs in the gRNA-expressing iPSCs.

Cells were transduced using lentivirus according to the following conditions: (a) control lentivirus conferring puromycin resistance only, (b) dSaCas9-2× VP64 and a non-frataxin targeting control gRNA, or (c) dSaCas9-2× VP64 and enhancer-targeting gRNA U. After transduction, cells were incubated for 2 days, then cultured in the presence of 1 μg/mL puromycin to enrich for transduced cells for an additional 2 days. Cells were then split using Accutase cell dissociation reagent (ThermoFisher #A1110501) and cultured for an additional 3 days prior to assessing frataxin expression levels. Approximately 60,000 cells per condition were harvested and assessed for relative frataxin mRNA expression levels by RT-qPCR as described above.

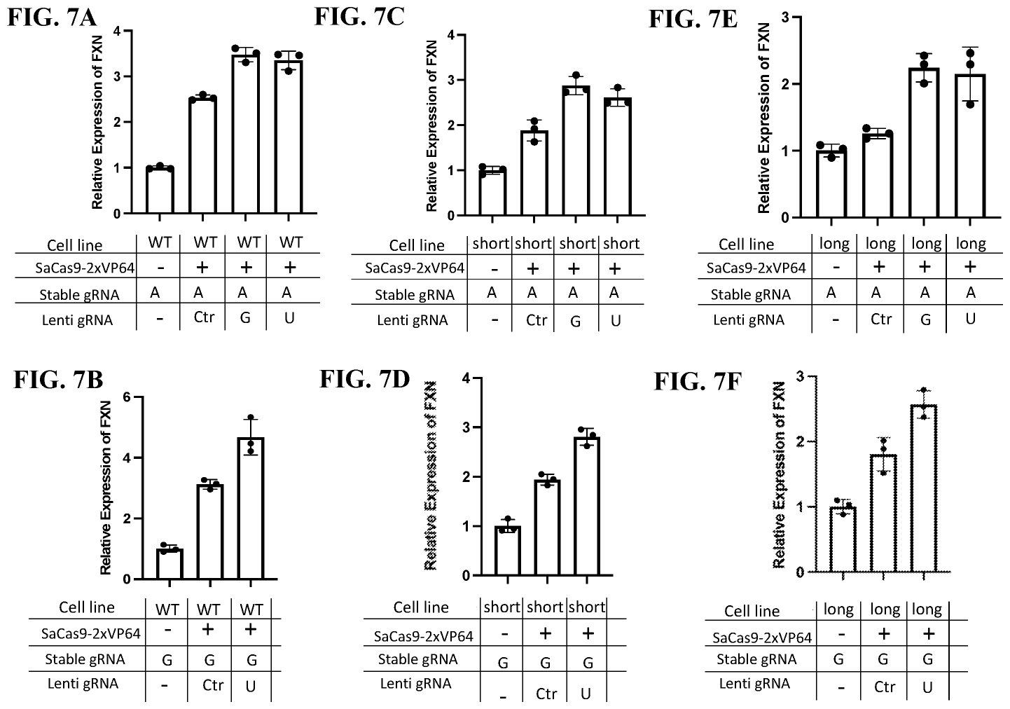

For all cell lines tested, cells transduced with dSaCas9-2× VP64 and gRNA U exhibited increased expression of frataxin in comparison to both control-transduced and dSaCas9-2× VP64/control gRNA-transduced cells, as shown for cell lines WT-gRNA A-iPSC (FIG. 7A), WT-gRNA G-iPSC (FIG. 7B), FA(short)-gRNA A-iPSC (FIG. 7C), FA(short)-gRNA G-iPSC (FIG. 7D), FA(long)-gRNA A-iPSC (FIG. 7E), and FA(long)-gRNA G-iPSC (FIG. 7F).

The results indicate that an exemplary enhancer region-targeting gRNA (gRNA U) facilitates further transcriptional activation of frataxin when paired with exemplary promoter-targeting gRNAs (gRNA A or gRNA G), both in the context of WT-iPSCs and FA-iPSCs with short and long trinucleotide repeats. These results support the utility of the exemplary DNA-targeting system and gRNAs in restoring frataxin expression levels in FA patient cells and potential treatment of FA.

### Example 4: Rescue of Frataxin mRNA and Protein Expression in FA-iPSCs

Frataxin mRNA and protein expression were compared between WT-iPSCs and FA-iPSCs. FA-iPSCs were transduced with dCas9-transcriptional activator fusion protein and gRNAs, and assessed for rescue of frataxin expression in comparison to WT-iPSCs.

A. Rescue of Frataxin mRNA Expression with Promoter- and Enhancer-Targeting gRNAs in FA-iPSCs

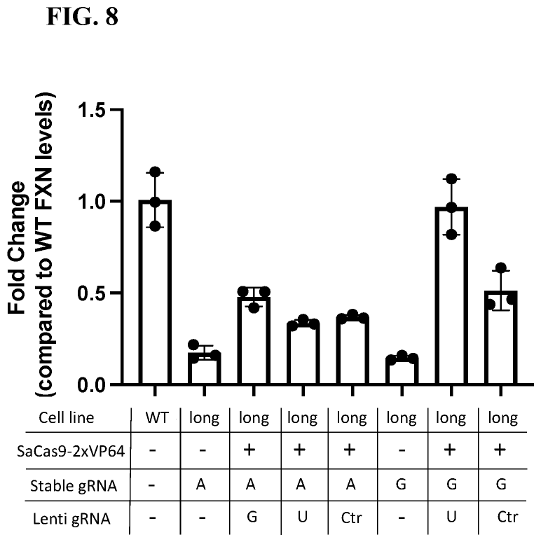

FA-iPSC cell lines containing long trinucleotide repeats and transduced with gRNAs, (FA(long)-gRNA A-iPSC and FA(long)-gRNA G-iPSC described in Table E2 above), exhibited severely reduced frataxin mRNA expression levels in comparison to WT-iPSCs, as assessed by RT-qPCR (approximately 15% WT expression for both lines) (FIG. 8). Transduction of FA(long)-gRNA A-iPSC and FA(long)-gRNA G-iPSC with dSaCas9-2× VP64 and a control gRNA increased frataxin mRNA expression to approximately 35% and approximately 50% WT levels, respectively. Transduction of FA(long)-gRNA G-iPSC with dSaCas9-2× VP64 and enhancer-targeted gRNA U further increased frataxin mRNA expression to levels comparable to WT (FIG. 8).

B. Rescue of Frataxin Protein Expression with Promoter- and Enhancer-Targeting gRNAs in FA-iPSCs

Modulation of frataxin protein expression using promoter- and enhancer-targeting gRNAs in WT- and FA-iPSCs was assessed. For protein measurement experiments, cells were transduced and incubated for 2 days, then cultured in the presence of 1 μg/mL puromycin to enrich for transduced cells for an additional 2 days. Cells were then split using Accutase cell dissociation reagent (ThermoFisher #A1110501) and cultured for an additional 3 days prior to assessing frataxin protein expression levels. Approximately 0.5 to 1.0 million cells per condition were harvested and assessed for frataxin protein expression levels. Frataxin protein was measured by a Human Frataxin ELISA Kit (abcam #ab176112) and normalized to total protein as assessed by Bicinchoninic acid (BCA) assay.

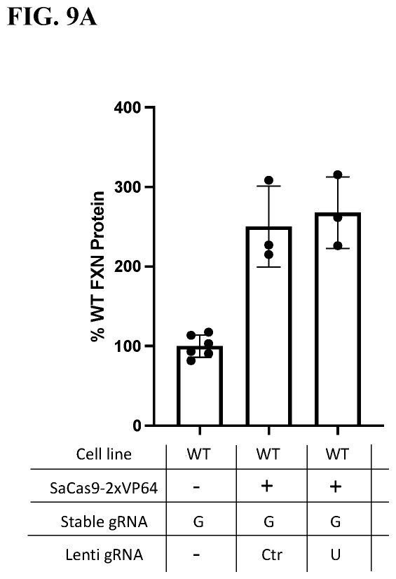

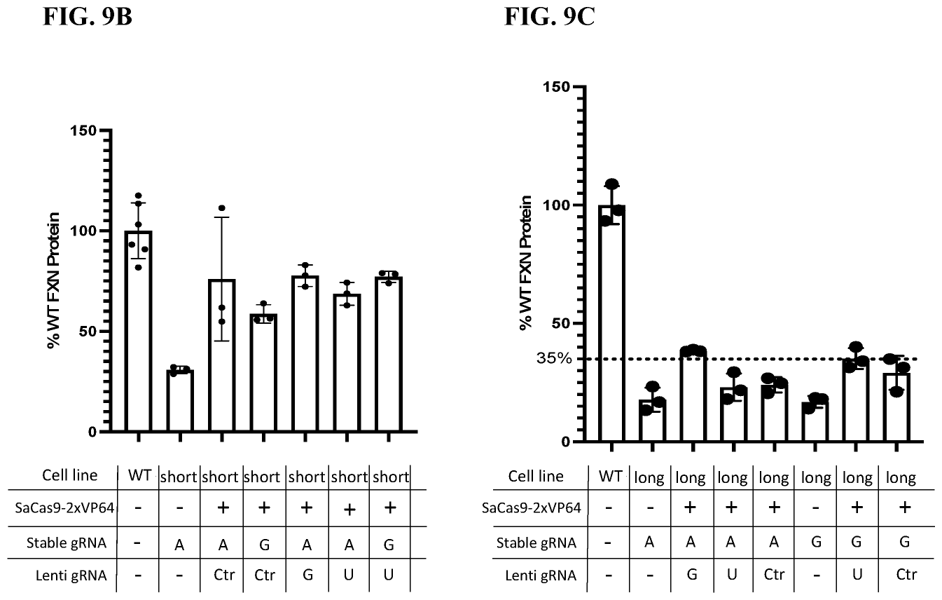

WT-gRNA G-iPSCs were (a) left untreated, (b) transduced with dSaCas9-2× VP64 and a control gRNA, or (c) transduced with dSaCas9-2× VP64 and the frataxin enhancer-targeting gRNA U. Transduced cells exhibited a greater than 2-fold increase in Frataxin protein expression (FIG. 9A).

FA(short)-gRNA A-iPSCs (as described in Table E2 above) expressed Frataxin protein at approximately 30% of WT expression levels (FIG. 9B). Transduction of dSaCas9-2× VP64 into FA(short)-gRNA A-iPSCs significantly increased frataxin protein expression to up to approximately 75% of WT levels. In addition, FA(short)-gRNA G-iPSCs transduced with dSaCas9-2× VP64 and enhancer-targeted gRNA U exhibited increased expression of frataxin protein in comparison to the same cell line transduced with dSaCas9-2× VP64 and a control gRNA (FIG. 9B). These results indicate that gRNA U facilitates further increase of frataxin protein expression when paired with a promoter-targeting gRNA, rescuing the reduced frataxin protein expression in the context of FA-associated trinucleotide repeat expansion.

FA(long)-gRNA A-iPSC and FA(long)-gRNA G-iPSC cells each expressed Frataxin protein at approximately 15% of WT expression levels (FIG. 9C). Transduction of FA(long)-gRNA A-iPSCs with dSaCas9-2× VP64 and gRNA G increased frataxin protein expression to approximately 30% of WT levels. Transduction of FA(long)-gRNA G-iPSC with dSaCas9-2× VP64 and gRNA U also increased frataxin protein expression to approximately 35% of WT levels. Cells in both of these conditions expressed frataxin at higher levels than cells expressing dSaCas9-2× VP64 and a single gRNA (FIG. 9C).

These results showed a substantial increase of frataxin mRNA and protein expression in FA-iPSCs, facilitated by combinations of gRNAs targeted to promoter and enhancer regions of the frataxin gene, in some cases, to a level similar to WT levels. Friedreich's ataxia (FA) patients expressing frataxin protein above 35% of the frataxin protein levels observed in individuals with no frataxin trinucleotide repeat display reduced or no symptoms of FA. The results showing increased frataxin protein expression to at least 35% of WT levels in FA-iPSCs with long trinucleotide repeats supports therapeutic utility of the dCas9-transcriptional activator fusion protein and exemplary gRNAs targeting frataxin.

### Example 5: Large-Scale Screen for dSaCas9 Transcriptional Activator and Repressor Fusion Proteins

A library of plasmids was generated encoding fusion proteins comprising fragments of nuclear localized proteins, fused to the N-terminus or C-terminus of dCas9. The libraries were screened separately in a pooled format to identify activators and repressors of frataxin in combination with an exemplary promoter-targeting gRNA.

A. dSaCas9-Effector Screen

A library of plasmids was generated encoding fusion proteins comprising protein fragments of nuclear localized proteins fused to the N-terminus of dSaCas9. A second library was generated with the protein fragments fused to the C-terminus of dSaCas9. The two libraries were each screened separately in a pooled format using iPSCs expressing an exemplary frataxin promoter-targeting gRNA A, as described in Example 3B above (WT-gRNA A-iPSC cells).

dSaCas9 fusion proteins comprising nuclear localized protein fragments facilitating transcriptional activation or repression of frataxin expression were identified as follows. WT-gRNA A-iPSC cells were transduced using lentivirus with the pooled dSaCas9-effector library at a multiplicity of infection (MOI) of 0.4 and incubated for 1 day. Cells were then cultured in the presence of 1 μg/mL puromycin to enrich for transduced cells for 7 days. Cells were then fixed with 4% PFA at room temperature, permeabilized with MeOH overnight at −20° C. and labeled with a mouse monoclonal anti-frataxin fluorophore-conjugated antibody (abcam ab156033). Cells were sorted by flow cytometry into 2 populations comprising the top 10% and bottom 10% of cells based on frataxin protein expression.

Sequencing was performed to identify effectors (i.e., nuclear localized protein fragments) enriched in the frataxin-high population (activators) and frataxin-low population (repressors). Genomic DNA was isolated from the sorted populations. Targeted PCR was performed to amplify the protein fragment sequences and append sequencing adapters. Each sample was barcoded separately. Samples were then sequenced using an Illumina MiSeq System. Three replicates of the frataxin-high and frataxin-low population were compared using DEseq2, a method for detecting differentially expressed transcripts.

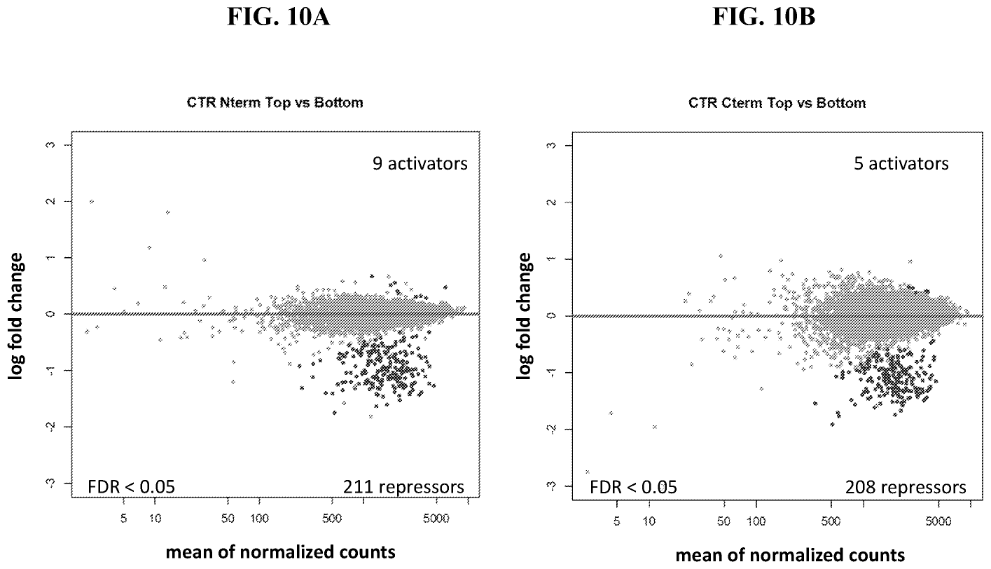

Based on sequencing results, 9 activators and 211 repressors of frataxin expression were identified from the N-terminal protein fragment screen (FIG. 10A). 5 activators and 208 repressors were identified from the C-terminal protein fragment screen (FIG. 10B). 3 of the activator domains were identified in both screens, including fragments from the proteins NCOA2, NCOA3, and PYGO1.

### B. Effector Validation

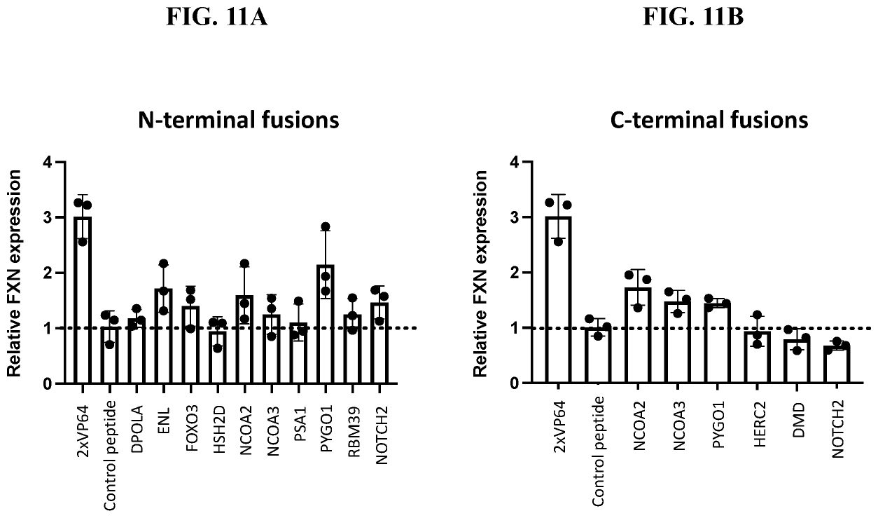

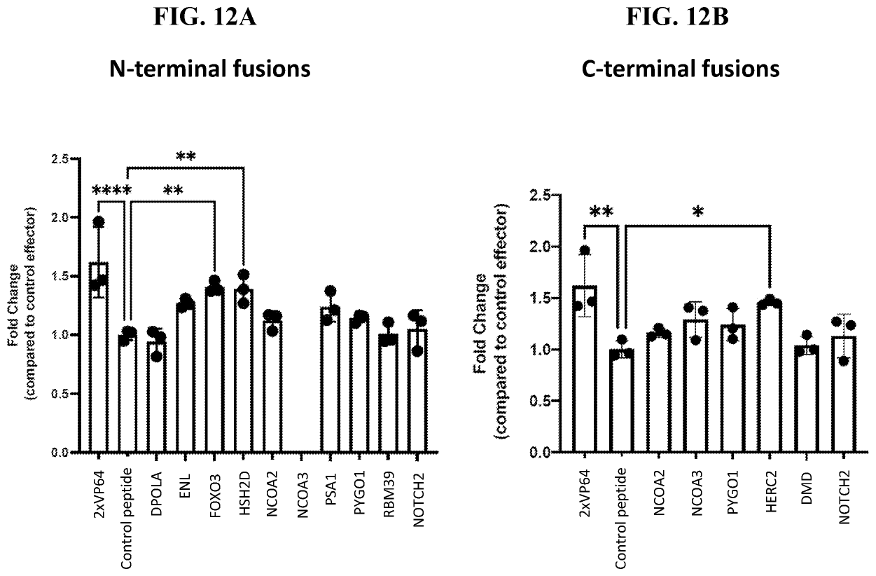

dSaCas9-effector fusion proteins (i.e., comprising nuclear localized protein fragments) identified in the screen described above were verified individually. WT-gRNA A-iPSCs were transduced with dSaCas9-2× VP64 (positive control) or the identified fusion proteins containing activators from the screen described above. Resulting frataxin mRNA expression was measured by RT-qPCR as described above, in comparison to control cells transduced with a dSaCas9 fusion protein having no effect on frataxin expression levels as determined in the effector screen (N-terminal fusions: FIG. 11A; C-terminal fusions: FIG. 11B). dSaCas9 effector fusion proteins comprising fragments of the genes NCOA2 and PYOG1 led to upregulation of frataxin mRNA. The results showed that several of the identified dCas9-effector fusion proteins and an exemplary frataxin promoter-targeting gRNA resulted in an increase in frataxin mRNA expression. dSaCas9-effector fusion proteins were also tested in FA(long)-gRNA A-iPSCs (N-terminal fusions: FIG. 12A; C-terminal fusions: FIG. 12B). dSaCas9 effector fusion proteins comprising fragments of the genes FOXO, HSH2D, and HERC2 led to upregulation of frataxin mRNA.

The results show that dCas9 effector fusion proteins identified in the screen and an exemplary frataxin promoter-targeting gRNA can facilitate upregulation of frataxin expression in WT- and FA-iPSCs. These results support the utility of the exemplary DNA-targeting system and gRNAs in restoring frataxin expression levels in FA patient cells and potential treatment of FA.

### Example 6: Rescue of Frataxin Expression in Differentiated Cells Derived from FA-iPSCs

Various differentiated cell types derived from FA-iPSCs, including cardiomyocytes and neurons, were transduced with a dCas9-transcriptional activator fusion protein and gRNAs, and assessed for rescue of frataxin expression in comparison to WT-iPSC-derived cardiomyocytes and neurons, respectively.

### A. Rescue of Frataxin Expression in FA-iPSC-Derived Cardiomyocytes

FA-iPSC-derived cardiomyocytes harboring expanded GAA trinucleotide repeats (867/867) were transduced with dSaCas-2× VP64 and frataxin (FXN) promoter and enhancer-targeting gRNAs (gRNA A; gRNA G; gRNA A+gRNA G; or gRNA G+gRNA U) using lentivirus, generally as described in Examples 1-4 above. Control cells were transduced with a non-targeting gRNA (NT gRNA), or with a puromycin resistance cassette alone (puro control). WT-iPSC-derived cardiomyocytes were included as controls to assess rescue of FXN expression (WT line+puro control). Transduced cells were enriched via puromycin selection from day 3 to day 10 post-transduction, and cells were harvested for analysis on day 10.

FXN mRNA expression levels were assessed by RT-qPCR (normalized to GAPDH mRNA) in comparison to FXN mRNA expression levels in WT control cells.

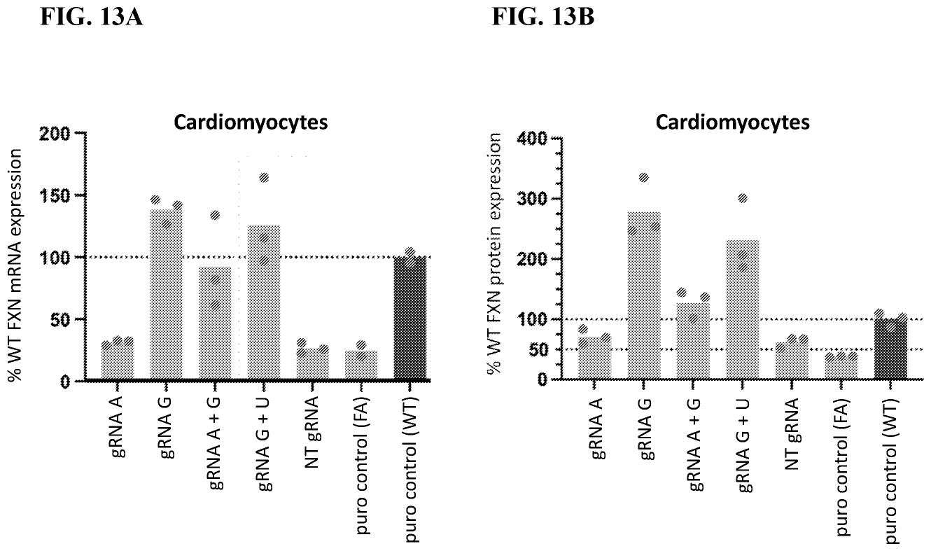

As shown in FIG. 13A, FA-iPSC-derived cardiomyocytes exhibited substantially reduced FXN mRNA expression levels in comparison to WT controls. In comparison, transduction of FA-iPSC-derived cardiomyocytes with dSaCas9-2× VP64 and FXN promoter- or enhancer-targeting gRNAs (gRNA G; gRNA A+gRNA G; or gRNA G+gRNA U) increased FXN mRNA expression in comparison to control FA-iPSC-derived cardiomyocytes (puro control). Furthermore, FXN mRNA expression was increased to levels comparable to or greater than levels in WT control cells.

FXN protein expression levels were assessed by ELISA (normalized to total protein levels) in comparison to FXN protein expression levels in WT control cells.

As shown in FIG. 13B, FA-iPSC-derived cardiomyocytes exhibited substantially reduced FXN protein expression levels in comparison to WT controls. However, transduction of FA-iPSC-derived cardiomyocytes with dSaCas9-2× VP64 and FXN promoter- and enhancer-targeting gRNAs (gRNA A; gRNA G; gRNA A+gRNA G; or gRNA G+gRNA U) increased FXN protein expression in comparison to control FA-iPSC-derived cardiomyocytes (puro control). Furthermore, FXN protein expression was increased to levels comparable to or greater than levels in WT control cells.

These results showed a substantial increase of frataxin mRNA and protein expression in FA-iPSC-derived cardiomyocytes, when transduced with a dCas9-transcriptional activator and individual or combinations of gRNAs targeted to promoter and enhancer regions of the frataxin gene, including to levels comparable to or higher than WT levels.

### B. Rescue of Frataxin Expression in FA-iPSC-Derived Neurons

FA-iPSC-derived neurons harboring expanded GAA trinucleotide repeats (867/867) were transduced with dSaCas-2× VP64 and FXN promoter and enhancer-targeting gRNAs (gRNA A; gRNA G; gRNA A+gRNA G; or gRNA G+gRNA U) using lentivirus, generally as described in Examples 1-4 above. Control cells were transduced with a non-targeting gRNA (NT gRNA), or with a puromycin resistance cassette alone (puro control). WT-iPSC-derived neurons were included as controls to assess rescue of FXN expression (WT line+puro control). Transduced cells were enriched via puromycin selection from day 3 to day 10 post-transduction, and cells were harvested for analysis on day 10.

Similar to the assessment in differentiated cardiomyocytes, FXN mRNA expression levels were assessed by RT-qPCR (normalized to GAPDH) in comparison to FXN mRNA expression levels in WT control cells.

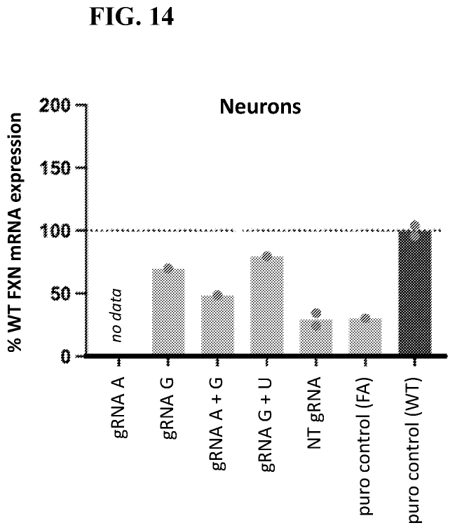

As shown in FIG. 14, FA-iPSC-derived neurons exhibited substantially reduced FXN mRNA expression levels in comparison to WT controls. Transduction of FA-iPSC-derived neurons with dSaCas9-2× VP64 and FXN promoter- and enhancer-targeting gRNAs (gRNA A; gRNA G; gRNA A+gRNA G; or gRNA G+gRNA U) increased FXN mRNA expression in comparison to control FA-iPSC-derived neurons (puro control). Furthermore, FXN mRNA expression was increased to levels comparable to levels in WT control cells.

These results showed a substantial increase of frataxin mRNA expression in FA-iPSC-derived neurons, when transduced with a dCas9-transcriptional activator and individual or combinations of gRNAs targeted to promoter and enhancer regions of the frataxin gene, including to levels comparable to or higher than WT levels.

### C. Conclusion

These results together showed that transduction with a dCas9-transcriptional activator and individual or combinations of gRNAs targeted to promoter and enhancer regions leads to a substantial increase of frataxin mRNA expression in FA-iPSC-derived cardiomyocytes and neurons, and frataxin protein expression in FA-iPSC-derived cardiomyocytes, including rescuing mRNA or protein expression levels to levels comparable to or higher than WT levels.

In some cases, Friedreich's ataxia (FA) patients expressing frataxin protein above 35% of levels observed in individuals with no FA display reduced or no symptoms of FA. The results showing increased frataxin expression to at least 35% or higher of WT levels in FA-iPSC-derived cardiomyocytes supports the therapeutic utility of the dCas9-transcriptional activator fusion protein and exemplary gRNAs targeting frataxin, including in heart cells, such as cardiomyocytes. These results further support therapeutic utility of the exemplary dCas9-transcriptional activator fusion protein and exemplary gRNAs targeting frataxin, including in various cell types, including heart cells, such as cardiomyocytes, and in neurons.

### Example 7: Transcriptional Activation of Frataxin in Non-Human Primate Fibroblasts

Cells from different organisms, such as non-human primates, were transduced with a dCas9-transcriptional activator fusion protein and FXN promoter-targeting gRNAs, and assessed for transcriptional activation of FXN.

Non-human primate fibroblast cell lines, including those shown in Table E3, were obtained and cultured for the following experiments.

TABLE E3
Non-human primate cell lines

| Cell line | | | GAPDH | FXN |
|---|---|---|---|---|
| Source/ | | Cell | Taqman | Taqman |
| Cat.# | Species | type | qPCR Probes | qPCR Probes |
| Coriell | Macaca | Fibro- | Rh02621745_g1 | Rh02827119_g1 |
| Institute, | mulatta | blast | | |
| Cat#: | (Rhesus | | | |
| AG07107 | monkey) | | | |
| Coriell | Macaca | Fibro- | Mf04392546_g1 | Mf04363465_g1 |
| Institute, | fascicularis | blast | | |
| Cat#: | (Crab-eating | | | |
| AG21329 | monkey) | | | |

Lentiviral vectors for expression in the non-human primate cell lines were designed and cloned, each comprising nucleic acid sequences encoding dSaCas9-2× VP64 and gRNA A, gRNA G, both gRNA A and gRNA G, described in Examples 1-4 above, which also target conserved sequences of the non-human primate FXN locus, or a non-targeting gRNA (NT gRNA). Vectors further encoded a puromycin resistance cassette for enrichment of transduced cells. The vectors were transduced into the non-human primate cell lines, and transduced cells were enriched using 6 μg/mL puromycin from day 2 to day 7 post-transduction.

Cells were collected 7 days post-transduction and analyzed by RT-qPCR for expression of FXN mRNA. RT-qPCR Taqman probes for FXN expression (and control GAPDH expression) are shown in Table E3 for the non-human primate cell lines. FXN expression was compared to expression in control cells transduced with a puromycin resistance cassette alone.

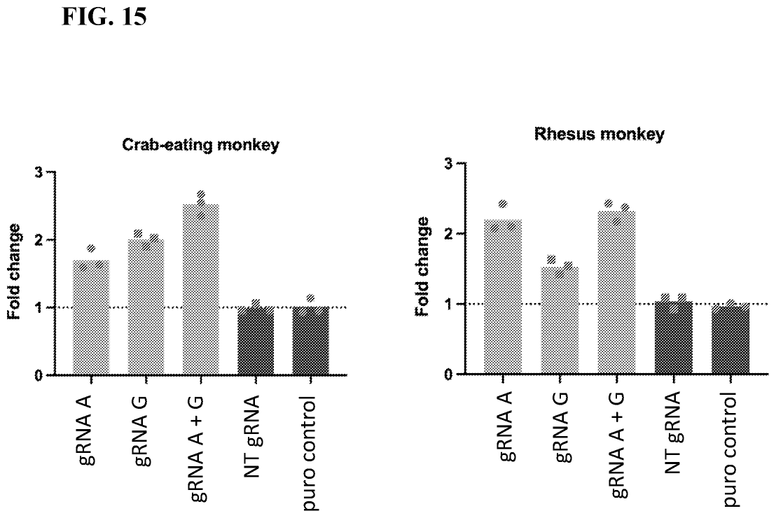

As shown in FIG. 15, transduction of non-human primate cell lines with the dCas9-transcriptional activator fusion protein and FXN promoter-targeting gRNAs led to increased transcription of FXN in comparison to control cells. These results further support the utility of the exemplary dCas9-transcriptional activator fusion protein and exemplary gRNAs targeting frataxin, in increasing transcription of FXN in different cell types from different organisms.

### Example 8: Transcriptional Activation of Frataxin In Vivo in a Friedreich's Ataxia (FA) Mouse Model

A humanized mouse model for Friedreich's ataxia (FA) was treated with a dCas9-transcriptional activator fusion protein and frataxin (FXN) targeting gRNAs, and assessed for in vivo transcriptional activation of FXN.

Adeno-associated virus (AAV) vectors were designed and generated to evaluate the in vivo effect of the dCas9-transcriptional activator fusion protein and different exemplary gRNAs described in Examples 1-4 above. Each single-stranded AAV9 (ssAAV9) vector included nucleic acid sequences encoding dSaCas9-2× VP64 and one of the following: 1) promoter-targeting gRNA A (see Example 1 above); 2) promoter-targeting gRNA G (see Example 1 above); 3) enhancer-targeting gRNA U (see Example 2C above); and 4) a non-targeting gRNA (NT gRNA). For the combination test group, ssAAV9 vectors encoding gRNA U and ssAAV9 vectors encoding gRNA G were combined and tested along with the other groups.

6-8 week old male and female humanized FA mouse model, Fxn^{em2.1Lutzy} Tg(FXN)YG8Pook/800J (n=4M/4F, “FA mouse model”) and healthy control wild type humanized mouse model Fxn^{em2.1Lutzy} Tg(FXN)Y47Pook/J (n=4M/4F, “Healthy”) were intravenously injected with one of the ssAAV9 viral vectors at a total of 1×10¹⁴ viral genomes (vg)/kg (0.5×10¹⁴ vg/kg each for the vectors containing gRNA G and gRNA U for the combination group). Mice were euthanized 30 days after infusion, and heart and liver tissues were collected, weighted and snap frozen in liquid nitrogen. The frozen heart and liver tissues were homogenized and assayed for human FXN protein by ELISA (Abcam© cat #ab176112). To assess biodistribution of the AAV9 vectors, DNA was extracted from the heart and liver tissue samples and AAV9 vector genome quantification by droplet digital (ddPCR). The heart tissue homogenate was also assessed by Succinate Dehydrogenase (SDH) in vitro enzymatic activity assay (Abcam© cat #ab228560).

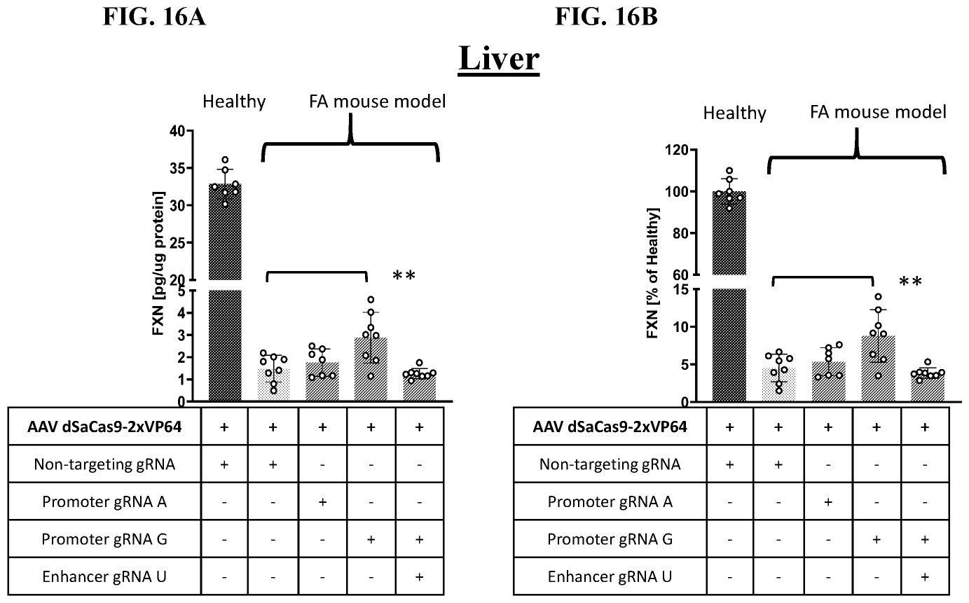

In the liver of the FA mouse model, as shown in FIG. 16A (expressed as FXN protein pg per μg of loaded protein into the assay [pg/μg protein]) and FIG. 16B (normalized to the average FXN protein levels from healthy control mice), treatment with dSaCas9-2× VP64 and FXN promoter-targeting gRNAs or a combination of FXN promoter- and enhancer-targeting gRNAs significantly increased FXN protein levels compared to treatment with dSaCas9-2× VP64 and non-targeting gRNA control.

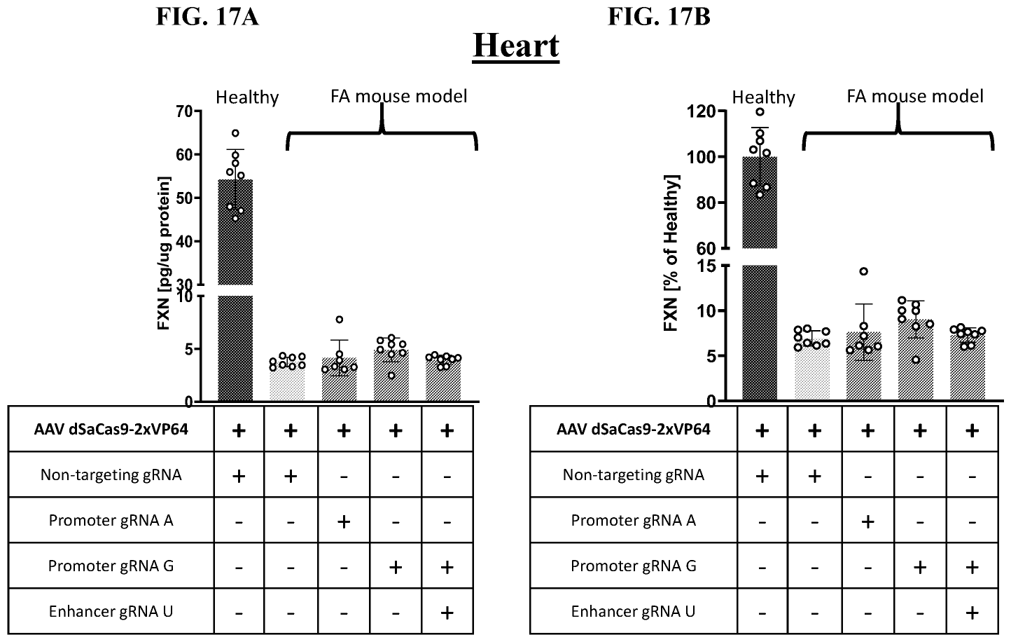

In the heart of the FA mouse model, as shown in FIG. 17A (expressed as FXN protein pg per μg of loaded protein into the assay [pg/μg protein]) and FIG. 17B (normalized to the average FXN protein levels from healthy control mice), treatment with dSaCas9-2× VP64 and FXN promoter-targeting gRNAs or a combination of FXN promoter- and enhancer-targeting gRNAs resulted in an increase in FXN protein levels compared to treatment with dSaCas9-2× VP64 and non-targeting gRNA control.

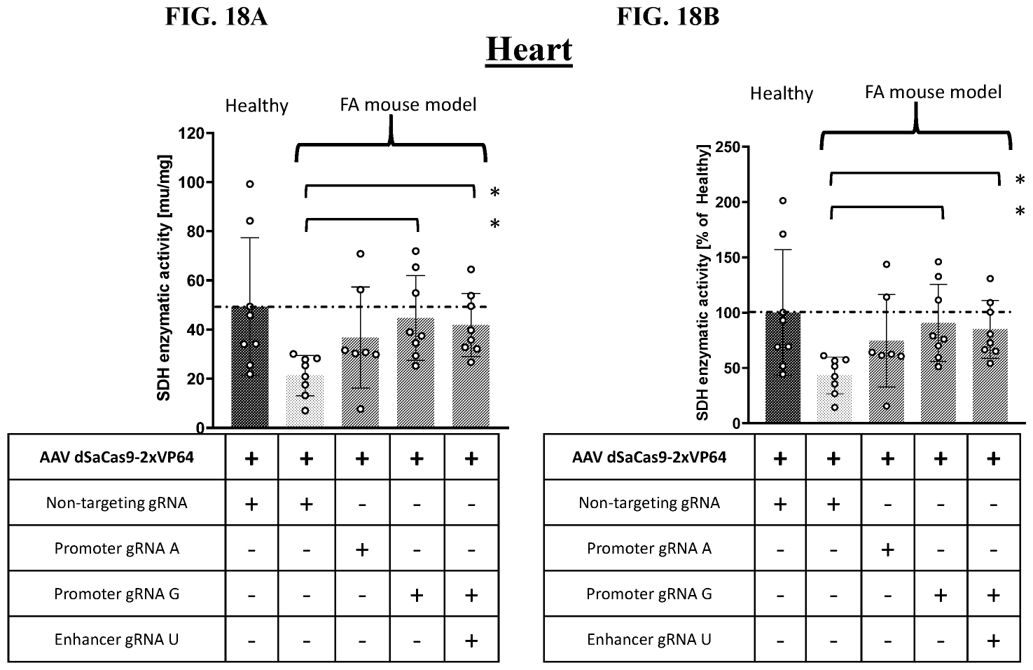

SDH enzymatic activity was assessed in the heart homogenates of the FA mouse model, as reduced SDH activity impairs mitochondrial function and has been reported to occur in heart autopsies from FA patients. As shown in FIG. 18A (expressed as mU of SDH activity per mg loaded protein into the assay [mu/mg]) and FIG. 18B (normalized to the SDH activity from healthy control mice), treatment with dSaCas9-2× VP64 and FXN promoter-targeting gRNAs or a combination of FXN promoter- and enhancer-targeting gRNAs significantly increased SDH enzymatic activity compared treatment with dSaCas9-2× VP64 and non-targeting gRNA control, rescuing the reduced SDH enzymatic activity to near the levels observed in the healthy control mice.

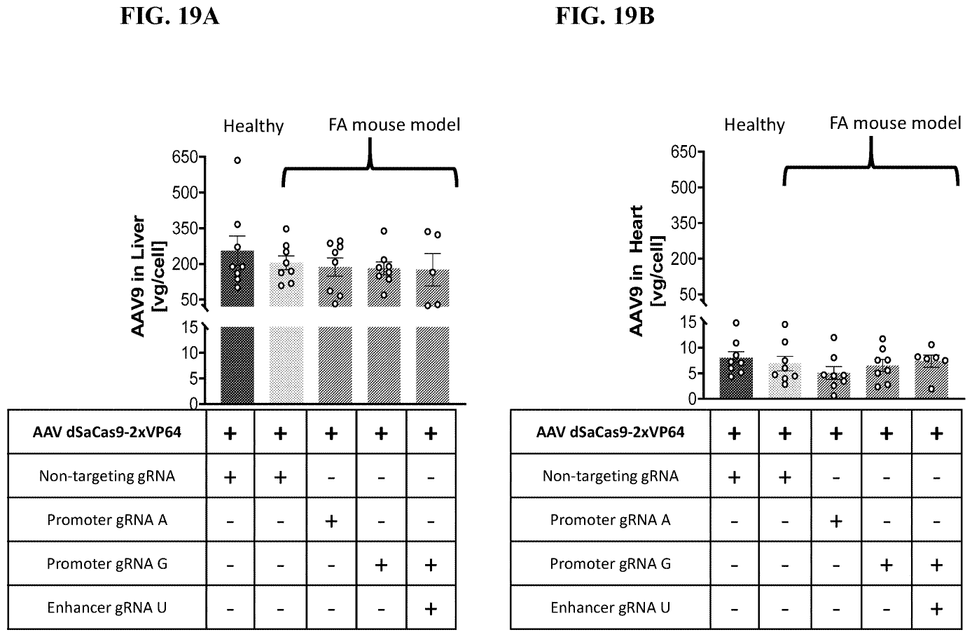

Biodistribution of the administered AAV9 vectors in are shown in FIG. 19A (liver) and FIG. 19B (heart). The biodistribution patterns of the administered vectors were consistent with the known tropism of AAV9, and no difference was observed based on mouse (FA model or healthy) or encoded gRNA.

The results showed that in vivo treatment with an exemplary dCas9-transcriptional activator fusion protein and exemplary gRNAs targeting different target sites in frataxin led to an increase in expression of FXN protein in various tissues, and also rescued the SDH enzymatic activity levels in the heart, in a mouse model of FA. The results further support the utility of the exemplary dCas9-transcriptional activator fusion protein and exemplary gRNAs targeting frataxin in an in vivo treatment for FA.

### Example 9: ZFP-Mediated Transcriptional Activation of FXN

Fusion proteins containing DNA-targeting domains based on zinc finger proteins (ZFP) that target the frataxin (FXN) locus were designed, generated, and assessed for their effect in activation of FXN in cells.

ZFP-based DNA-targeting domains targeting regulatory elements of FXN, including promoter-targeting and enhancer-targeting ZFP DNA-targeting domains, were designed, based on available methods for designing ZFP targeting specific target sequences. Exemplary ZFP DNA-targeting domains target sequences within the genomic coordinates human genome assembly GRCh38 (hg38) chr9:68,940,179-69,205,519. Exemplary FXN promoter-targeting ZFP DNA-targeting domains target sequences within the genomic coordinates hg38 chr9:69,034,622-69,036,670; chr9:69,034,900-69,035,900; or chr9:69,035,300-69,035,900. Exemplary FXN enhancer-targeting ZFP DNA-targeting domains target sequences within the genomic coordinates hg38 chr9:69,027,282-69,028,497 or chr9:69,027,615-69,028,101. The exemplary genomic regions specified above contained multiple sequentially tiled target sites, designing ZFPs targeting one of the tiled target sites in the region. Fusion proteins were designed, each comprising one of the designed ZFP DNA-targeting domains fused to a transcriptional activator, such as 2 copies of the transcriptional activator VP64 (FXN-targeting ZFP-2× VP64).

Viral vectors, including lentiviral vectors, were designed and cloned, each comprising nucleic acid sequences encoding a FXN-targeting ZFP-2× VP64. Vectors further encoded a selectable marker (e.g. puromycin resistance cassette). FA-iPSCs, generally described in Examples 1-4 above, were transduced with each individual FXN-targeting ZFP-2× VP64 fusion protein or combinations thereof, and enriched for transduced cells (e.g. using puromycin selection). Negative control cells were transduced with nucleic acid sequences encoding a non-targeting ZFP-2× VP64 fusion protein, the FXN-targeting ZFP DNA-targeting domains without VP64, or the selectable marker alone. Cells were harvested and analyzed for FXN mRNA expression by RT-qPCR and FXN protein expression by ELISA, in comparison to negative control cells. In some examples, expression was also compared to WT-iPSCs transduced with the selectable marker alone. In some cases, plasmids encoding the designed FXN-targeting ZFP-transcriptional activator fusion protein are transcribed in vitro to generate mRNA encoding the fusion proteins, and transiently transfected directly into FA-iPSCs.

FA-iPSCs transduced with one of the FXN-targeting ZFP-transcriptional activator fusion proteins were assessed for activation of frataxin expression using RT-qPCR (mRNA) and ELISA (protein).

Increased FXN expression in FA-iPSCs in comparison to negative control cells, and FXN protein expression in FA-iPSCs of at least 35% WT-iPSC levels, support the utility of fusion proteins comprising FXN-targeting ZFP DNA-targeting domains and transcriptional activators, for targeted transcriptional activation of FXN and increase in FXN protein expression, including in connection with therapies for FA.

### Example 10: TALE-Mediated Transcriptional Activation of FXN

Fusion proteins containing DNA-targeting domains based on transcription activator-like effector (TALE) binding domains that target the frataxin (FXN) locus are designed, generated, and assessed for their effect in activation of FXN in cells.

TALE-based DNA-targeting domains targeting regulatory elements of FXN, including promoter-targeting and enhancer-targeting TALE DNA-targeting domains, are designed, based on available methods for designing TALE targeting specific target sequences. Exemplary TALE DNA-targeting domains target sequences within the genomic coordinates human genome assembly GRCh38 (hg38) chr9:68,940,179-69,205,519. Exemplary FXN promoter-targeting TALE DNA-targeting domains target sequences within the genomic coordinates hg38 chr9:69,034,622-69,036,670; chr9:69,034,900-69,035,900; or chr9:69,035,300-69,035,900. Exemplary FXN enhancer-targeting TALE DNA-targeting domains target sequences within the genomic coordinates hg38 chr9:69,027,282-69,028,497 or chr9:69,027,615-69,028,101. The exemplary genomic regions specified above contained multiple sequentially tiled target sites, designing TALEs targeting one of the tiled target sites in the region. Fusion proteins are designed, each comprising one of the designed TALE DNA-targeting domains fused to a transcriptional activator, such as 2 copies of the transcriptional activator VP64 (FXN-targeting TALE-2× VP64).

Viral vectors, including lentiviral vectors, are designed and cloned, each comprising nucleic acid sequences encoding a FXN-targeting TALE-2× VP64. Vectors further encoded a selectable marker (e.g. puromycin resistance cassette). FA-iPSCs, generally described in Examples 1-4 above, are transduced with each individual FXN-targeting TALE-2× VP64 fusion protein or combinations thereof, and enriched for transduced cells (e.g. using puromycin selection). Negative control cells are transduced with nucleic acid sequences encoding a non-targeting TALE-2× VP64 fusion protein, the FXN-targeting TALE DNA-targeting domains without VP64, or the selectable marker alone. Cells are harvested and analyzed for FXN mRNA expression by RT-qPCR and FXN protein expression by ELISA, in comparison to negative control cells. In some examples, expression is also compared to WT-iPSCs transduced with the selectable marker alone.

FA-iPSCs transduced with one of the FXN-targeting TALE-transcriptional activator fusion proteins are assessed for activation of frataxin expression using RT-qPCR (mRNA) and ELISA (protein).

Increased FXN expression in FA-iPSCs in comparison to negative control cells, and FXN protein expression in FA-iPSCs of at least 35% WT-iPSC levels, support the utility of fusion proteins comprising FXN-targeting TALE DNA-targeting domains and transcriptional activators, for targeted transcriptional activation of FXN and increase in FXN protein expression, including in connection with therapies for FA.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Seuences

| # | SEQUENCE | ANNOTATION |
|---|---|---|
| 1 | TCACACAGCTAGGAAGTGGG | target site A or |
| | | gRNA spacer |
| 2 | GAGGCTGCTTGGCCGCCGGT | target site B or |
| | | gRNA spacer |
| 3 | GCCCGCTCCGCCCTCCAGCG | target site C or |
| | | gRNA spacer |
| 4 | AGCCTGCTTTGTGCAAAGCA | target site D or |
| | | gRNA spacer |
| 5 | CCGGCCGATGACGCGCCGCGGG | target site E or |
| | | gRNA spacer |
| 6 | GGCCGGCTACTGCGCGGCGCCC | target site F or |
| | | gRNA spacer |
| 7 | ACCTCTAGCTGCTCCCCCACAG | target site G or |
| | | gRNA spacer |
| 8 | CCGCCCGCTCCGCCCTCCAGCG | target site H or |
| | | gRNA spacer |
| 9 | GCCCGAGAGTCCACATGCTGCT | target site I or |
| | | gRNA spacer |
| 10 | GCTCTCCATTTTTGTTAAATGC | target site J or |
| | | gRNA spacer |
| 11 | CTGCTGTAAACCCATACCGG | target site K or |
| | | gRNA spacer |
| 12 | GCAGAGTACAGATTTACACA | target site L or |
| | | gRNA spacer |
| 13 | CAAGGGAGACTGCAGCCTGG | target site M or |
| | | gRNA spacer |
| 14 | AAGCTGGGAAGTTCTTCCTG | target site N or |
| | | gRNA spacer |
| 15 | ATGCACGAATAGTGCTAAGC | target site O or |
| | | gRNA spacer |
| 16 | TACACAAGGCATCCGTCTCC | target site P or |
| | | gRNA spacer |
| 17 | GGCTGCTTGGCCGCCGGTAT | target site Q or |
| | | gRNA spacer |
| 18 | TTTAGAAGCGGCGGGCCACC | target site R or |
| | | gRNA spacer |
| 19 | AATTGAGGCTGCTTGGCCGC | target site S or |
| | | gRNA spacer |
| 20 | GCAAAGCACGGAGTGCAACC | target site T or |
| | | gRNA spacer |
| 21 | TGCATGGGACAACCACCACGA | target site U or |
| | | gRNA spacer |
| 22 | UCACACAGCUAGGAAGUGGG | gRNA A |
| | | spacer RNA |
| 23 | GAGGCUGCUUGGCCGCCGGU | gRNA B spacer |
| | | RNA |
| 24 | GCCCGCUCCGCCCUCCAGCG | gRNA C spacer |
| | | RNA |
| 25 | AGCCUGCUUUGUGCAAAGCA | gRNA D |
| | | spacer RNA |
| 26 | CCGGCCGAUGACGCGCCGCGGG | gRNA E spacer |
| | | RNA |
| 27 | GGCCGGCUACUGCGCGGCGCCC | gRNA F spacer |
| | | RNA |
| 28 | ACCUCUAGCUGCUCCCCCACAG | gRNA G |
| | | spacer RNA |
| 29 | CCGCCCGCUCCGCCCUCCAGCG | gRNA H |
| | | spacer RNA |
| 30 | GCCCGAGAGUCCACAUGCUGCU | gRNA I spacer |
| | | RNA |
| 31 | GCUCUCCAUUUUUGUUAAAUGC | gRNA J spacer |
| | | RNA |
| 32 | CUGCUGUAAACCCAUACCGG | gRNA K |
| | | spacer RNA |
| 33 | GCAGAGUACAGAUUUACACA | gRNA L spacer |
| | | RNA |
| 34 | CAAGGGAGACUGCAGCCUGG | gRNA M |
| | | spacer RNA |
| 35 | AAGCUGGGAAGUUCUUCCUG | gRNA N |
| | | spacer RNA |
| 36 | AUGCACGAAUAGUGCUAAGC | gRNA O |
| | | spacer RNA |
| 37 | UACACAAGGCAUCCGUCUCC | gRNA P spacer |
| | | RNA |
| 38 | GGCUGCUUGGCCGCCGGUAU | gRNA Q |
| | | spacer RNA |
| 39 | UUUAGAAGCGGCGGGCCACC | gRNA R spacer |
| | | RNA |
| 40 | AAUUGAGGCUGCUUGGCCGC | gRNA S spacer |
| | | RNA |
| 41 | GCAAAGCACGGAGUGCAACC | gRNA T spacer |
| | | RNA |
| 42 | UGCAUGGGACAACCACCACGA | gRNA U |
| | | spacer RNA |
| 43 | GTTTTAGTACTCTGGAAACAGAATCTACTAAAACAAGGCAAAATGCCGTGTTTATCTCG | SaCas9 gRNA |
| | TCAACTTGTTGGCGAGA | scaffold DNA |
| 44 | GUUUUAGUACUCUGGAAACAGAAUCUACUAAAACAAGGCAAAAUGCCGUGUUUAUCUCG | SaCas9 gRNA |
| | UCAACUUGUUGGCGAGA | scaffold |
| 45 | GTTTAAGAGCTATGCTGGAAACAGCATAGCAAGTTTAAATAAGGCTAGTCCGTTATCAA | SpCas9 gRNA |
| | CTTGAAAAAGTGGCACCGAGTCGGTGC | scaffold DNA |
| 46 | GUUUAAGAGCUAUGCUGGAAACAGCAUAGCAAGUUUAAAUAAGGCUAGUCCGUUAUCAA | SpCas9 gRNA |
| | CUUGAAAAAGUGGCACCGAGUCGGUGC | scaffold |
| 47 | UCACACAGCUAGGAAGUGGGGUUUUAGUACUCUGGAAACAGAAUCUACUAAAACAAGGC | gRNA A |
| | AAAAUGCCGUGUUUAUCUCGUCAACUUGUUGGCGAGA | |
| 48 | GAGGCUGCUUGGCCGCCGGUGUUUUAGUACUCUGGAAACAGAAUCUACUAAAACAAGGC | gRNA B |
| | AAAAUGCCGUGUUUAUCUCGUCAACUUGUUGGCGAGA | |
| 49 | GCCCGCUCCGCCCUCCAGCGGUUUUAGUACUCUGGAAACAGAAUCUACUAAAACAAGGC | gRNA C |
| | AAAAUGCCGUGUUUAUCUCGUCAACUUGUUGGCGAGA | |
| 50 | AGCCUGCUUUGUGCAAAGCAGUUUUAGUACUCUGGAAACAGAAUCUACUAAAACAAGGC | gRNA D |
| | AAAAUGCCGUGUUUAUCUCGUCAACUUGUUGGCGAGA | |
| 51 | CCGGCCGAUGACGCGCCGCGGGGUUUUAGUACUCUGGAAACAGAAUCUACUAAAACAAG | gRNA E |
| | GCAAAAUGCCGUGUUUAUCUCGUCAACUUGUUGGCGAGA | |
| 52 | GGCCGGCUACUGCGCGGCGCCCGUUUUAGUACUCUGGAAACAGAAUCUACUAAAACAAG | gRNA F |
| | GCAAAAUGCCGUGUUUAUCUCGUCAACUUGUUGGCGAGA | |
| 53 | ACCUCUAGCUGCUCCCCCACAGGUUUUAGUACUCUGGAAACAGAAUCUACUAAAACAAG | gRNA G |
| | GCAAAAUGCCGUGUUUAUCUCGUCAACUUGUUGGCGAGA | |
| 54 | CCGCCCGCUCCGCCCUCCAGCGGUUUUAGUACUCUGGAAACAGAAUCUACUAAAACAAG | gRNA H |
| | GCAAAAUGCCGUGUUUAUCUCGUCAACUUGUUGGCGAGA | |
| 55 | GCCCGAGAGUCCACAUGCUGCUGUUUUAGUACUCUGGAAACAGAAUCUACUAAAACAAG | gRNA I |
| | GCAAAAUGCCGUGUUUAUCUCGUCAACUUGUUGGCGAGA | |
| 56 | GCUCUCCAUUUUUGUUAAAUGCGUUUUAGUACUCUGGAAACAGAAUCUACUAAAACAAG | gRNA J |
| | GCAAAAUGCCGUGUUUAUCUCGUCAACUUGUUGGCGAGA | |
| 57 | CUGCUGUAAACCCAUACCGGGUUUAAGAGCUAUGCUGGAAACAGCAUAGCAAGUUUAAA | gRNA K |
| | UAAGGCUAGUCCGUUAUCAACUUGAAAAAGUGGCACCGAGUCGGUGC | |
| 58 | GCAGAGUACAGAUUUACACAGUUUAAGAGCUAUGCUGGAAACAGCAUAGCAAGUUUAAA | gRNA L |
| | UAAGGCUAGUCCGUUAUCAACUUGAAAAAGUGGCACCGAGUCGGUGC | |
| 59 | CAAGGGAGACUGCAGCCUGGGUUUAAGAGCUAUGCUGGAAACAGCAUAGCAAGUUUAAA | gRNA M |
| | UAAGGCUAGUCCGUUAUCAACUUGAAAAAGUGGCACCGAGUCGGUGC | |
| 60 | AAGCUGGGAAGUUCUUCCUGGUUUAAGAGCUAUGCUGGAAACAGCAUAGCAAGUUUAAA | gRNA N |
| | UAAGGCUAGUCCGUUAUCAACUUGAAAAAGUGGCACCGAGUCGGUGC | |
| 61 | AUGCACGAAUAGUGCUAAGCGUUUAAGAGCUAUGCUGGAAACAGCAUAGCAAGUUUAAA | gRNA O |
| | UAAGGCUAGUCCGUUAUCAACUUGAAAAAGUGGCACCGAGUCGGUGC | |
| 62 | UACACAAGGCAUCCGUCUCCGUUUAAGAGCUAUGCUGGAAACAGCAUAGCAAGUUUAAA | gRNA P |
| | UAAGGCUAGUCCGUUAUCAACUUGAAAAAGUGGCACCGAGUCGGUGC | |
| 63 | GGCUGCUUGGCCGCCGGUAUGUUUAAGAGCUAUGCUGGAAACAGCAUAGCAAGUUUAAA | gRNA Q |
| | UAAGGCUAGUCCGUUAUCAACUUGAAAAAGUGGCACCGAGUCGGUGC | |
| 64 | UUUAGAAGCGGCGGGCCACCGUUUAAGAGCUAUGCUGGAAACAGCAUAGCAAGUUUAAA | gRNA R |
| | UAAGGCUAGUCCGUUAUCAACUUGAAAAAGUGGCACCGAGUCGGUGC | |
| 65 | AAUUGAGGCUGCUUGGCCGCGUUUAAGAGCUAUGCUGGAAACAGCAUAGCAAGUUUAAA | gRNA S |
| | UAAGGCUAGUCCGUUAUCAACUUGAAAAAGUGGCACCGAGUCGGUGC | |
| 66 | GCAAAGCACGGAGUGCAACCGUUUAAGAGCUAUGCUGGAAACAGCAUAGCAAGUUUAAA | gRNA T |
| | UAAGGCUAGUCCGUUAUCAACUUGAAAAAGUGGCACCGAGUCGGUGC | |
| 67 | UGCAUGGGACAACCACCACGAGUUUUAGUACUCUGGAAACAGAAUCUACUAAAACAAGG | gRNA U |
| | CAAAAUGCCGUGUUUAUCUCGUCAACUUGUUGGCGAGA | |
| 68 | GACATTGATTATTGACTAGTTATTAATAGTAATCAATTACGGGGTCATTAGTTCATAGC | gRNA VP64- |
| | CCATATATGGAGTTCCGCGTTACATAACTTACGGTAAATGGCCCGCCTGGCTGACCGCC | dSaCas9-NLS- |
| | CAACGACCCCCGCCCATTGACGTCAATAATGACGTATGTTCCCATAGTAACGCCAATAG | VP64 vector |
| | GGACTTTCCATTGACGTCAATGGGTGGAGTATTTACGGTAAACTGCCCACTTGGCAGTA | |
| | CATCAAGTGTATCATATGCCAAGTACGCCCCCTATTGACGTCAATGACGGTAAATGGCC | |
| | CGCCTGGCATTATGCCCAGTACATGACCTTATGGGACTTTCCTACTTGGCAGTACATCT | |
| | ACGTATTAGTCATCGCTATTACCATGGTGATGCGGTTTTGGCAGTACATCAATGGGCGT | |
| | GGATAGCGGTTTGACTCACGGGGATTTCCAAGTCTCCACCCCATTGACGTCAATGGGAG | |
| | TTTGTTTTGGCACCAAAATCAACGGGACTTTCCAAAATGTCGTAACAACTCCGCCCCAT | |
| | TGACGCAAATGGGCGGTAGGCGTGTACGGTGGGAGGTCTATATAAGCAGCGCGTTTTGC | |
| | CTGTACTGGGTCTCTCTGGTTAGACCAGATCTGAGCCTGGGAGCTCTCTGGCTAACTAG | |
| | GGAACCCACTGCTTAAGCCTCAATAAAGCTTGCCTTGAGTGCTTCAAGTAGTGTGTGCC | |
| | CGTCTGTTGTGTGACTCTGGTAACTAGAGATCCCTCAGACCCTTTTAGTCAGTGTGGAA | |
| | AATCTCTAGCAGTGGCGCCCGAACAGGGACTTGAAAGCGAAAGGGAAACCAGAGGAGCT | |
| | CTCTCGACGCAGGACTCGGCTTGCTGAAGCGCGCACGGCAAGAGGCGAGGGGCGGCGAC | |
| | TGGTGAGTACGCCAAAAATTTTGACTAGCGGAGGCTAGAAGGAGAGAGATGGGTGCGAG | |
| | AGCGTCAGTATTAAGCGGGGGAGAATTAGATCGCGATGGGAAAAAATTCGGTTAAGGCC | |
| | AGGGGGAAAGAAAAAATATAAATTAAAACATATAGTATGGGCAAGCAGGGAGCTAGAAC | |
| | GATTCGCAGTTAATCCTGGCCTGTTAGAAACATCAGAAGGCTGTAGACAAATACTGGGA | |
| | CAGCTACAACCATCCCTTCAGACAGGATCAGAAGAACTTAGATCATTATATAATACAGT | |
| | AGCAACCCTCTATTGTGTGCATCAAAGGATAGAGATAAAAGACACCAAGGAAGCTTTAG | |
| | ACAAGATAGAGGAAGAGCAAAACAAAAGTAAGACCACCGCACAGCAAGCGGCCGCTGAT | |
| | CTTCAGACCTGGAGGAGGAGATATGAGGGACAATTGGAGAAGTGAATTATATAAATATA | |
| | AAGTAGTAAAAATTGAACCATTAGGAGTAGCACCCACCAAGGCAAAGAGAAGAGTGGTG | |
| | CAGAGAGAAAAAAGAGCAGTGGGAATAGGAGCTTTGTTCCTTGGGTTCTTGGGAGCAGC | |
| | AGGAAGCACTATGGGCGCAGCGTCAATGACGCTGACGGTACAGGCCAGACAATTATTGT | |
| | CTGGTATAGTGCAGCAGCAGAACAATTTGCTGAGGGCTATTGAGGCGCAACAGCATCTG | |
| | TTGCAACTCACAGTCTGGGGCATCAAGCAGCTCCAGGCAAGAATCCTGGCTGTGGAAAG | |
| | ATACCTAAAGGATCAACAGCTCCTGGGGATTTGGGGTTGCTCTGGAAAACTCATTTGCA | |
| | CCACTGCTGTGCCTTGGAATGCTAGTTGGAGTAATAAATCTCTGGAACAGATTTGGAAT | |
| | CACACGACCTGGATGGAGTGGGACAGAGAAATTAACAATTACACAAGCTTAATACACTC | |
| | CTTAATTGAAGAATCGCAAAACCAGCAAGAAAAGAATGAACAAGAATTATTGGAATTAG | |
| | ATAAATGGGCAAGTTTGTGGAATTGGTTTAACATAACAAATTGGCTGTGGTATATAAAA | |
| | TTATTCATAATGATAGTAGGAGGCTTGGTAGGTTTAAGAATAGTTTTTGCTGTACTTTC | |
| | TATAGTGAATAGAGTTAGGCAGGGATATTCACCATTATCGTTTCAGACCCACCTCCCAA | |
| | CCCCGAGGGGACCCGACAGGCCCGAAGGAATAGAAGAAGAAGGTGGAGAGAGAGACAGA | |
| | GACAGATCCATTCGATTAGTGAACGGATCGGCACTGCGTGCGCCAATTCTGCAGACAAA | |
| | TGGCAGTATTCATCCACAATTTTAAAAGAAAAGGGGGGATTGGGGGGTACAGTGCAGGG | |
| | GAAAGAATAGTAGACATAATAGCAACAGACATACAAACTAAAGAATTACAAAAACAAAT | |
| | TACAAAAATTCAAAATTTTCGGGTTTATTACAGGGACAGCAGAGATCCAGTTTGGTTAA | |
| | TTAAAAAAAAATCTCGCCAACAAGTTGACGAGATAAACACGGCATTTTGCCTTGTTTTA | |
| | GTAGATTCTGTTTCCAGAGTACTAAAACAGAGACGATTTACAACCGTCTCCGGTGTTTC | |
| | GTCCTTTCCACAAGATATATAAAGCCAAGAAATCGAAATACTTTCAAGTTACGGTAAGC | |
| | ATATGATAGTCCATTTTAAAACATAATTTTAAAACTGCAAACTACCCAAGAAATTATTA | |
| | CTTTCTACGTCACGTATTTTGTACTAATATCTTTGTGTTTACAGTCAAATTAATTCCAA | |
| | TTATCTCTCTAACAGCCTTGTATCGTATATGCAAATATGAAGGAATCATGGGAAATAGG | |
| | CCCTCTCTAGAGATATCCCTAGCGGCCTCCGCGCCGGGTTTTGGCGCCTCCCGCGGGCG | |
| | CCCCCCTCCTCACGGCGAGCGCTGCCACGTCAGACGAAGGGCGCAGCGAGCGTCCTGAT | |
| | CCTTCCGCCCGGACGCTCAGGACAGCGGCCCGCTGCTCATAAGACTCGGCCTTAGAACC | |
| | CCAGTATCAGCAGAAGGACATTTTAGGACGGGACTTGGGTGACTCTAGGGCACTGGTTT | |
| | TCTTTCCAGAGAGCGGAACAGGCGAGGAAAAGTAGTCCCTTCTCGGCGATTCTGCGGAG | |
| | GGATCTCCGTGGGGCGGTGAACGCCGATGATTATATAAGGACGCGCCGGGTGTGGCACA | |
| | GCTAGTTCCGTCGCAGCCGGGATTTGGGTCGCGGTTCTTGTTTGTGGATCGCTGTGATC | |
| | GTCACTTGGTGAGTAGCGGGCTGCTGGGCTGGCCGGGGCTTTCGTGGCCGCCGGGCCGC | |
| | TCGGTGGGACGGAAGCGTGTGGAGAGACCGCCAAGGGCTGTAGTCTGGGTCCGCGAGCA | |
| | AGGTTGCCCTGAACTGGGGGTTGGGGGGAGCGCAGCAAAATGGCGGCTGTTCCCGAGTC | |
| | TTGAATGGAAGACGCTTGTGAGGCGGGCTGTGAGGTCGTTGAAACAAGGTGGGGGGCAT | |
| | GGTGGGCGGCAAGAACCCAAGGTCTTGAGGCCTTCGCTAATGCGGGAAAGCTCTTATTC | |
| | GGGTGAGATGGGCTGGGGCACCATCTGGGGACCCTGACGTGAAGTTTGTCACTGACTGG | |
| | AGAACTCGGTTTGTCGTCTGTTGCGGGGGCGGCAGTTATGGCGGTGCCGTTGGGCAGTG | |
| | CACCCGTACCTTTGGGAGCGCGCGCCCTCGTCGTGTCGTGACGTCACCCGTTCTGTTGG | |
| | CTTATAATGCAGGGTGGGGCCACCTGCCGGTAGGTGTGCGGTAGGCTTTTCTCCGTCGC | |
| | AGGACGCAGGGTTCGGGCCTAGGGTAGGCTCTCCTGAATCGACAGGCGCCGGACCTCTG | |
| | GTGAGGGGAGGGATAAGTGAGGCGTCAGTTTCTTTGGTCGGTTTTATGTACCTATCTTC | |
| | TTAAGTAGCTGAAGCTCCGGTTTTGAACTATGCGCTCGGGGTTGGCGAGTGTGTTTTGT | |
| | GAAGTTTTTTAGGCACCTTTTGAAATGTAATCATTTGGGTCAATATGTAATTTTCAGTG | |
| | TTAGACTAGTAAATTGTCCGCTAAATTCTGGCCGTTTTTGGCTTTTTTGTTAGACGACC | |
| | GGTTCTAGCGCGCTGCCACCATGGACTACAAAGACCATGACGGTGATTATAAAGATCAT | |
| | GACATCGATTACAAGGATGACGATGACAAGCACggtccgcgggctgacgcattggacga | |
| | ttttgatctggatatgctgggaagtgacgccctcgatgattttgaccttgacatgcttg | |
| | gttcggatgcccttgatgactttgacctcgacatgctcggcagtgacgcccttgatgat | |
| | ttcgacctggacatgGTTAACCCAAAGAAGAAGCGGAAGGTCGGTATCCACGGAGTCCC | |
| | AGCAGCCAAGCGGAACTACATCCTGGGCCTGGCCATCGGCATCACCAGCGTGGGCTACG | |
| | GCATCATCGACTACGAGACACGGGACGTGATCGATGCCGGCGTGCGGCTGTTCAAAGAG | |
| | GCCAACGTGGAAAACAACGAGGGCAGGCGGAGCAAGAGAGGCGCCAGAAGGCTGAAGCG | |
| | GCGGAGGCGGCATAGAATCCAGAGAGTGAAGAAGCTGCTGTTCGACTACAACCTGCTGA | |
| | CCGACCACAGCGAGCTGAGCGGCATCAACCCCTACGAGGCCAGAGTGAAGGGCCTGAGC | |
| | CAGAAGCTGAGCGAGGAAGAGTTCTCTGCCGCCCTGCTGCACCTGGCCAAGAGAAGAGG | |
| | CGTGCACAACGTGAACGAGGTGGAAGAGGACACCGGCAACGAGCTGTCCACCAAAGAGC | |
| | AGATCAGCCGGAACAGCAAGGCCCTGGAAGAGAAATACGTGGCCGAACTGCAGCTGGAA | |
| | CGGCTGAAGAAAGACGGCGAAGTGCGGGGCAGCATCAACAGATTCAAGACCAGCGACTA | |
| | CGTGAAAGAAGCCAAACAGCTGCTGAAGGTGCAGAAGGCCTACCACCAGCTGGACCAGA | |
| | GCTTCATCGACACCTACATCGACCTGCTGGAAACCCGGCGGACCTACTATGAGGGACCT | |
| | GGCGAGGGCAGCCCCTTCGGCTGGAAGGACATCAAAGAATGGTACGAGATGCTGATGGG | |
| | CCACTGCACCTACTTCCCCGAGGAACTGCGGAGCGTGAAGTACGCCTACAACGCCGACC | |
| | TGTACAACGCCCTGAACGACCTGAACAATCTCGTGATCACCAGGGACGAGAACGAGAAG | |
| | CTGGAATATTACGAGAAGTTCCAGATCATCGAGAACGTGTTCAAGCAGAAGAAGAAGCC | |
| | CACCCTGAAGCAGATCGCCAAAGAAATCCTCGTGAACGAAGAGGATATTAAGGGCTACA | |
| | GAGTGACCAGCACCGGCAAGCCCGAGTTCACCAACCTGAAGGTGTACCACGACATCAAG | |
| | GACATTACCGCCCGGAAAGAGATTATTGAGAACGCCGAGCTGCTGGATCAGATTGCCAA | |
| | GATCCTGACCATCTACCAGAGCAGCGAGGACATCCAGGAAGAACTGACCAATCTGAACT | |
| | CCGAGCTGACCCAGGAAGAGATCGAGCAGATCTCTAATCTGAAGGGCTATACCGGCACC | |
| | CACAACCTGAGCCTGAAGGCCATCAACCTGATCCTGGACGAGCTGTGGCACACCAACGA | |
| | CAACCAGATCGCTATCTTCAACCGGCTGAAGCTGGTGCCCAAGAAGGTGGACCTGTCCC | |
| | AGCAGAAAGAGATCCCCACCACCCTGGTGGACGACTTCATCCTGAGCCCCGTCGTGAAG | |
| | AGAAGCTTCATCCAGAGCATCAAAGTGATCAACGCCATCATCAAGAAGTACGGCCTGCC | |
| | CAACGACATCATTATCGAGCTGGCCCGCGAGAAGAACTCCAAGGACGCCCAGAAAATGA | |
| | TCAACGAGATGCAGAAGCGGAACCGGCAGACCAACGAGCGGATCGAGGAAATCATCCGG | |
| | ACCACCGGCAAAGAGAACGCCAAGTACCTGATCGAGAAGATCAAGCTGCACGACATGCA | |
| | GGAAGGCAAGTGCCTGTACAGCCTGGAAGCCATCCCTCTGGAAGATCTGCTGAACAACC | |
| | CCTTCAACTATGAGGTGGACCACATCATCCCCAGAAGCGTGTCCTTCGACAACAGCTTC | |
| | AACAACAAGGTGCTCGTGAAGCAGGAAGAAgcCAGCAAGAAGGGCAACCGGACCCCATT | |
| | CCAGTACCTGAGCAGCAGCGACAGCAAGATCAGCTACGAAACCTTCAAGAAGCACATCC | |
| | TGAATCTGGCCAAGGGCAAGGGCAGAATCAGCAAGACCAAGAAAGAGTATCTGCTGGAA | |
| | GAACGGGACATCAACAGGTTCTCCGTGCAGAAAGACTTCATCAACCGGAACCTGGTGGA | |
| | TACCAGATACGCCACCAGAGGCCTGATGAACCTGCTGCGGAGCTACTTCAGAGTGAACA | |
| | ACCTGGACGTGAAAGTGAAGTCCATCAATGGCGGCTTCACCAGCTTTCTGCGGCGGAAG | |
| | TGGAAGTTTAAGAAAGAGCGGAACAAGGGGTACAAGCACCACGCCGAGGACGCCCTGAT | |
| | CATTGCCAACGCCGATTTCATCTTCAAAGAGTGGAAGAAACTGGACAAGGCCAAAAAAG | |
| | TGATGGAAAACCAGATGTTCGAGGAAAAGCAGGCCGAGAGCATGCCCGAGATCGAAACC | |
| | GAGCAGGAGTACAAAGAGATCTTCATCACCCCCCACCAGATCAAGCACATTAAGGACTT | |
| | CAAGGACTACAAGTACAGCCACCGGGTGGACAAGAAGCCTAATAGAGAGCTGATTAACG | |
| | ACACCCTGTACTCCACCCGGAAGGACGACAAGGGCAACACCCTGATCGTGAACAATCTG | |
| | AACGGCCTGTACGACAAGGACAATGACAAGCTGAAAAAGCTGATCAACAAGAGCCCCGA | |
| | AAAGCTGCTGATGTACCACCACGACCCCCAGACCTACCAGAAACTGAAGCTGATTATGG | |
| | AACAGTACGGCGACGAGAAGAATCCCCTGTACAAGTACTACGAGGAAACCGGGAACTAC | |
| | CTGACCAAGTACTCCAAAAAGGACAACGGCCCCGTGATCAAGAAGATTAAGTATTACGG | |
| | CAACAAACTGAACGCCCATCTGGACATCACCGACGACTACCCCAACAGCAGAAACAAGG | |
| | TCGTGAAGCTGTCCCTGAAGCCCTACAGATTCGACGTGTACCTGGACAATGGCGTGTAC | |
| | AAGTTCGTGACCGTGAAGAATCTGGATGTGATCAAAAAAGAAAACTACTACGAAGTGAA | |
| | TAGCAAGTGCTATGAGGAAGCTAAGAAGCTGAAGAAGATCAGCAACCAGGCCGAGTTTA | |
| | TCGCCTCCTTCTACAACAACGATCTGATCAAGATCAACGGCGAGCTGTATAGAGTGATC | |
| | GGCGTGAACAACGACCTGCTGAACCGGATCGAAGTGAACATGATCGACATCACCTACCG | |
| | CGAGTACCTGGAAAACATGAACGACAAGAGGCCCCCCAGGATCATTAAGACAATCGCCT | |
| | CCAAGACCCAGAGCATTAAGAAGTACAGCACAGACATTCTGGGCAACCTGTATGAAGTG | |
| | AAATCTAAGAAGCACCCTCAGATCATCAAAAAGGGCAAAAGGCCGGCGGCCACGAAAAA | |
| | GGCCGGCCAGGCAAAAAAGAAAAAGGctagCcgcgccgacgcgctggacgatttcgatc | |
| | tcgacatgctgggttctgatgccctcgatgactttgacctggatatgttgggaagcgac | |
| | gcattggatgactttgatctggacatgctcggctccgatgctctggacgatttcgatct | |
| | cgatatgttaatcgctagcGCAACAAACTTCTCTCTGCTGAAACAAGCCGGAGATGTCG | |
| | AAGAGAATCCTGGACCGATGACCGAGTACAAGCCCACGGTGCGCCTCGCCACCCGCGAC | |
| | GACGTCCCCAGGGCCGTACGCACCCTCGCCGCCGCGTTCGCCGACTACCCCGCCACGCG | |
| | CCACACCGTCGATCCGGACCGCCACATCGAGCGGGTCACCGAGCTGCAAGAACTCTTCC | |
| | TCACGCGCGTCGGGCTCGACATCGGCAAGGTGTGGGTCGCGGACGACGGCGCCGCGGTG | |
| | GCGGTCTGGACCACGCCGGAGAGCGTCGAAGCGGGGGCGGTGTTCGCCGAGATCGGCCC | |
| | GCGCATGGCCGAGTTGAGCGGTTCCCGGCTGGCCGCGCAGCAACAGATGGAAGGCCTCC | |
| | TGGCGCCGCACCGGCCCAAGGAGCCCGCGTGGTTCCTGGCCACCGTCGGCGTGTCGCCC | |
| | GACCACCAGGGCAAGGGTCTGGGCAGCGCCGTCGTGCTCCCCGGAGTGGAGGCGGCCGA | |
| | GCGCGCCGGGGTGCCCGCCTTCCTGGAGACCTCCGCGCCCCGCAACCTCCCCTTCTACG | |
| | AGCGGCTCGGCTTCACCGTCACCGCCGACGTCGAGGTGCCCGAAGGACCGCGCACCTGG | |
| | TGCATGACCCGCAAGCCCGGTGCCTGAATTTAAATGAATTCGATATCAAGCTTATCGAT | |
| | AATCAACCTCTGGATTACAAAATTTGTGAAAGATTGACTGGTATTCTTAACTATGTTGC | |
| | TCCTTTTACGCTATGTGGATACGCTGCTTTAATGCCTTTGTATCATGCTATTGCTTCCC | |
| | GTATGGCTTTCATTTTCTCCTCCTTGTATAAATCCTGGTTGCTGTCTCTTTATGAGGAG | |
| | TTGTGGCCCGTTGTCAGGCAACGTGGCGTGGTGTGCACTGTGTTTGCTGACGCAACCCC | |
| | CACTGGTTGGGGCATTGCCACCACCTGTCAGCTCCTTTCCGGGACTTTCGCTTTCCCCC | |
| | TCCCTATTGCCACGGCGGAACTCATCGCCGCCTGCCTTGCCCGCTGCTGGACAGGGGCT | |
| | CGGCTGTTGGGCACTGACAATTCCGTGGTGTTGTCGGGGAAATCATCGTCCTTTCCTTG | |
| | GCTGCTCGCCTGTGTTGCCACCTGGATTCTGCGCGGGACGTCCTTCTGCTACGTCCCTT | |
| | CGGCCCTCAATCCAGCGGACCTTCCTTCCCGCGGCCTGCTGCCGGCTCTGCGGCCTCTT | |
| | CCGCGTCTTCGCCTTCGCCCTCAGACGAGTCGGATCTCCCTTTGGGCCGCCTCCCCGCA | |
| | TCGATACCGTCGACCTCGAGACCTAGAAAAACATGGAGCAATCACAAGTAGCAATACAG | |
| | CAGCTACCAATGCTGATTGTGCCTGGCTAGAAGCACAAGAGGAGGAGGAGGTGGGTTTT | |
| | CCAGTCACACCTCAGGTACCTTTAAGACCAATGACTTACAAGGCAGCTGTAGATCTTAG | |
| | CCACTTTTTAAAAGAAAAGGGGGGACTGGAAGGGCTAATTCACTCCCAACGAAGACAAG | |
| | ATATCCTTGATCTGTGGATCTACCACACACAAGGCTACTTCCCTGATTGGCAGAACTAC | |
| | ACACCAGGGCCAGGGATCAGATATCCACTGACCTTTGGATGGTGCTACAAGCTAGTACC | |
| | AGTTGAGCAAGAGAAGGTAGAAGAAGCCAATGAAGGAGAGAACACCCGCTTGTTACACC | |
| | CTGTGAGCCTGCATGGGATGGATGACCCGGAGAGAGAAGTATTAGAGTGGAGGTTTGAC | |
| | AGCCGCCTAGCATTTCATCACATGGCCCGAGAGCTGCATCCGGACTGTACTGGGTCTCT | |
| | CTGGTTAGACCAGATCTGAGCCTGGGAGCTCTCTGGCTAACTAGGGAACCCACTGCTTA | |
| | AGCCTCAATAAAGCTTGCCTTGAGTGCTTCAAGTAGTGTGTGCCCGTCTGTTGTGTGAC | |
| | TCTGGTAACTAGAGATCCCTCAGACCCTTTTAGTCAGTGTGGAAAATCTCTAGCAGGGC | |
| | CCGTTTAAACCCGCTGATCAGCCTCGACTGTGCCTTCTAGTTGCCAGCCATCTGTTGTT | |
| | TGCCCCTCCCCCGTGCCTTCCTTGACCCTGGAAGGTGCCACTCCCACTGTCCTTTCCTA | |
| | ATAAAATGAGGAAATTGCATCGCATTGTCTGAGTAGGTGTCATTCTATTCTGGGGGGTG | |
| | GGGTGGGGCAGGACAGCAAGGGGGAGGATTGGGAAGACAATAGCAGGCATGCTGGGGAT | |
| | GCGGTGGGCTCTATGGCTTCTGAGGCGGAAAGAACCAGCTGGGGCTCTAGGGGGTATCC | |
| | CCACGCGCCCTGTAGCGGCGCATTAAGCGCGGCGGGTGTGGTGGTTACGCGCAGCGTGA | |
| | CCGCTACACTTGCCAGCGCCCTAGCGCCCGCTCCTTTCGCTTTCTTCCCTTCCTTTCTC | |
| | GCCACGTTCGCCGGCTTTCCCCGTCAAGCTCTAAATCGGGGGCTCCCTTTAGGGTTCCG | |
| | ATTTAGTGCTTTACGGCACCTCGACCCCAAAAAACTTGATTAGGGTGATGGTTCACGTA | |
| | GTGGGCCATCGCCCTGATAGACGGTTTTTCGCCCTTTGACGTTGGAGTCCACGTTCTTT | |
| | AATAGTGGACTCTTGTTCCAAACTGGAACAACACTCAACCCTATCTCGGTCTATTCTTT | |
| | TGATTTATAAGGGATTTTGCCGATTTCGGCCTATTGGTTAAAAAATGAGCTGATTTAAC | |
| | AAAAATTTAACGCGAATTAATTCTGTGGAATGTGTGTCAGTTAGGGTGTGGAAAGTCCC | |
| | CAGGCTCCCCAGCAGGCAGAAGTATGCAAAGCATGCATCTCAATTAGTCAGCAACCAGG | |
| | TGTGGAAAGTCCCCAGGCTCCCCAGCAGGCAGAAGTATGCAAAGCATGCATCTCAATTA | |
| | GTCAGCAACCATAGTCCCGCCCCTAACTCCGCCCATCCCGCCCCTAACTCCGCCCAGTT | |
| | CCGCCCATTCTCCGCCCCATGGCTGACTAATTTTTTTTATTTATGCAGAGGCCGAGGCC | |
| | GCCTCTGCCTCTGAGCTATTCCAGAAGTAGTGAGGAGGCTTTTTTGGAGGCCTAGGCTT | |
| | TTGCAAAAAGCTCCCGGGAGCTTGTATATCCATTTTCGGATCTGATCAGCACGTGTTGA | |
| | CAATTAATCATCGGCATAGTATATCGGCATAGTATAATACGACAAGGTGAGGAACTAAA | |
| | CCATGGCCAAGTTGACCAGTGCCGTTCCGGTGCTCACCGCGCGCGACGTCGCCGGAGCG | |
| | GTCGAGTTCTGGACCGACCGGCTCGGGTTCTCCCGGGACTTCGTGGAGGACGACTTCGC | |
| | CGGTGTGGTCCGGGACGACGTGACCCTGTTCATCAGCGCGGTCCAGGACCAGGTGGTGC | |
| | CGGACAACACCCTGGCCTGGGTGTGGGTGCGCGGCCTGGACGAGCTGTACGCCGAGTGG | |
| | TCGGAGGTCGTGTCCACGAACTTCCGGGACGCCTCCGGGCCGGCCATGACCGAGATCGG | |
| | CGAGCAGCCGTGGGGGCGGGAGTTCGCCCTGCGCGACCCGGCCGGCAACTGCGTGCACT | |
| | TCGTGGCCGAGGAGCAGGACTGACACGTGCTACGAGATTTCGATTCCACCGCCGCCTTC | |
| | TATGAAAGGTTGGGCTTCGGAATCGTTTTCCGGGACGCCGGCTGGATGATCCTCCAGCG | |
| | CGGGGATCTCATGCTGGAGTTCTTCGCCCACCCCAACTTGTTTATTGCAGCTTATAATG | |
| | GTTACAAATAAAGCAATAGCATCACAAATTTCACAAATAAAGCATTTTTTTCACTGCAT | |
| | TCTAGTTGTGGTTTGTCCAAACTCATCAATGTATCTTATCATGTCTGTATACCGTCGAC | |
| | CTCTAGCTAGAGCTTGGCGTAATCATGGTCATAGCTGTTTCCTGTGTGAAATTGTTATC | |
| | CGCTCACAATTCCACACAACATACGAGCCGGAAGCATAAAGTGTAAAGCCTGGGGTGCC | |
| | TAATGAGTGAGCTAACTCACATTAATTGCGTTGCGCTCACTGCCCGCTTTCCAGTCGGG | |
| | AAACCTGTCGTGCCAGCTGCATTAATGAATCGGCCAACGCGCGGGGAGAGGCGGTTTGC | |
| | GTATTGGGCGCTCTTCCGCTTCCTCGCTCACTGACTCGCTGCGCTCGGTCGTTCGGCTG | |
| | CGGCGAGCGGTATCAGCTCACTCAAAGGCGGTAATACGGTTATCCACAGAATCAGGGGA | |
| | TAACGCAGGAAAGAACATGTGAGCAAAAGGCCAGCAAAAGGCCAGGAACCGTAAAAAGG | |
| | CCGCGTTGCTGGCGTTTTTCCATAGGCTCCGCCCCCCTGACGAGCATCACAAAAATCGA | |
| | CGCTCAAGTCAGAGGTGGCGAAACCCGACAGGACTATAAAGATACCAGGCGTTTCCCCC | |
| | TGGAAGCTCCCTCGTGCGCTCTCCTGTTCCGACCCTGCCGCTTACCGGATACCTGTCCG | |
| | CCTTTCTCCCTTCGGGAAGCGTGGCGCTTTCTCATAGCTCACGCTGTAGGTATCTCAGT | |
| | TCGGTGTAGGTCGTTCGCTCCAAGCTGGGCTGTGTGCACGAACCCCCCGTTCAGCCCGA | |
| | CCGCTGCGCCTTATCCGGTAACTATCGTCTTGAGTCCAACCCGGTAAGACACGACTTAT | |
| | CGCCACTGGCAGCAGCCACTGGTAACAGGATTAGCAGAGCGAGGTATGTAGGCGGTGCT | |
| | ACAGAGTTCTTGAAGTGGTGGCCTAACTACGGCTACACTAGAAGAACAGTATTTGGTAT | |
| | CTGCGCTCTGCTGAAGCCAGTTACCTTCGGAAAAAGAGTTGGTAGCTCTTGATCCGGCA | |
| | AACAAACCACCGCTGGTAGCGGTGGTTTTTTTGTTTGCAAGCAGCAGATTACGCGCAGA | |
| | AAAAAAGGATCTCAAGAAGATCCTTTGATCTTTTCTACGGGGTCTGACGCTCAGTGGAA | |
| | CGAAAACTCACGTTAAGGGATTTTGGTCATGAGATTATCAAAAAGGATCTTCACCTAGA | |
| | TCCTTTTAAATTAAAAATGAAGTTTTAAATCAATCTAAAGTATATATGAGTAAACTTGG | |
| | TCTGACAGTTACCAATGCITAATCAGTGAGGCACCTATCTCAGCGATCTGTCTATTTCG | |
| | TTCATCCATAGTTGCCTGACTCCCCGTCGTGTAGATAACTACGATACGGGAGGGCTTAC | |
| | CATCTGGCCCCAGTGCTGCAATGATACCGCGAGACCCACGCTCACCGGCTCCAGATTTA | |
| | TCAGCAATAAACCAGCCAGCCGGAAGGGCCGAGCGCAGAAGTGGTCCTGCAACTTTATC | |
| | CGCCTCCATCCAGTCTATTAATTGTTGCCGGGAAGCTAGAGTAAGTAGTTCGCCAGTTA | |
| | ATAGTTTGCGCAACGTTGTTGCCATTGCTACAGGCATCGTGGTGTCACGCTCGTCGTTT | |
| | GGTATGGCTTCATTCAGCTCCGGTTCCCAACGATCAAGGCGAGTTACATGATCCCCCAT | |
| | GTTGTGCAAAAAAGCGGTTAGCTCCTTCGGTCCTCCGATCGTTGTCAGAAGTAAGTTGG | |
| | CCGCAGTGTTATCACTCATGGTTATGGCAGCACTGCATAATTCTCTTACTGTCATGCCA | |
| | TCCGTAAGATGCTTTTCTGTGACTGGTGAGTACTCAACCAAGTCATTCTGAGAATAGTG | |
| | TATGCGGCGACCGAGTTGCTCTTGCCCGGCGTCAATACGGGATAATACCGCGCCACATA | |
| | GCAGAACTTTAAAAGTGCTCATCATTGGAAAACGTTCTTCGGGGCGAAAACTCTCAAGG | |
| | ATCTTACCGCTGTTGAGATCCAGTTCGATGTAACCCACTCGTGCACCCAACTGATCTTC | |
| | AGCATCTTTTACTTTCACCAGCGTTTCTGGGTGAGCAAAAACAGGAAGGCAAAATGCCG | |
| | CAAAAAAGGGAATAAGGGCGACACGGAAATGTTGAATACTCATACTCTTCCTTTTTCAA | |
| | TATTATTGAAGCATTTATCAGGGTTATTGTCTCATGAGCGGATACATATTTGAATGTAT | |
| | TTAGAAAAATAAACAAATAGGGGTTCCGCGCACATTTCCCCGAAAAGTGCCACCTGACG | |
| | TCGACGGATCGGGAGATCTCCCGATCCCCTATGGTGCACTCTCAGTACAATCTGCTCTG | |
| | ATGCCGCATAGTTAAGCCAGTATCTGCTCCCTGCTTGTGTGTTGGAGGTCGCTGAGTAG | |
| | TGCGCGAGCAAAATTTAAGCTACAACAAGGCAAGGCTTGACCGACAATTGCATGAAGAA | |
| | TCTGCTTAGGGTTAGGCGTTTTGCGCTGCTTCGCGATGTACGGGCCAGATATACGCGTT | |
| 69 | gacgcattggacgattttgatctggatatgctgggaagtgacgccctcgatgattttga | VP64- |
| | ccttgacatgcttggttcggatgcccttgatgactttgacctcgacatgctcggcagtg | dSaCas9-NLS- |
| | acgcccttgatgatttcgacctggacatgGTTAACCCAAAGAAGAAGCGGAAGGTCGGT | VP64 (nt) |
| | ATCCACGGAGTCCCAGCAGCCAAGCGGAACTACATCCTGGGCCTGGCCATCGGCATCAC | |
| | CAGCGTGGGCTACGGCATCATCGACTACGAGACACGGGACGTGATCGATGCCGGCGTGC | |
| | GGCTGTTCAAAGAGGCCAACGTGGAAAACAACGAGGGCAGGCGGAGCAAGAGAGGCGCC | |
| | AGAAGGCTGAAGCGGCGGAGGCGGCATAGAATCCAGAGAGTGAAGAAGCTGCTGTTCGA | |
| | CTACAACCTGCTGACCGACCACAGCGAGCTGAGCGGCATCAACCCCTACGAGGCCAGAG | |
| | TGAAGGGCCTGAGCCAGAAGCTGAGCGAGGAAGAGTTCTCTGCCGCCCTGCTGCACCTG | |
| | GCCAAGAGAAGAGGCGTGCACAACGTGAACGAGGTGGAAGAGGACACCGGCAACGAGCT | |
| | GTCCACCAAAGAGCAGATCAGCCGGAACAGCAAGGCCCTGGAAGAGAAATACGTGGCCG | |
| | AACTGCAGCTGGAACGGCTGAAGAAAGACGGCGAAGTGCGGGGCAGCATCAACAGATTC | |
| | AAGACCAGCGACTACGTGAAAGAAGCCAAACAGCTGCTGAAGGTGCAGAAGGCCTACCA | |
| | CCAGCTGGACCAGAGCTTCATCGACACCTACATCGACCTGCTGGAAACCCGGCGGACCT | |
| | ACTATGAGGGACCTGGCGAGGGCAGCCCCTTCGGCTGGAAGGACATCAAAGAATGGTAC | |
| | GAGATGCTGATGGGCCACTGCACCTACTTCCCCGAGGAACTGCGGAGCGTGAAGTACGC | |
| | CTACAACGCCGACCTGTACAACGCCCTGAACGACCTGAACAATCTCGTGATCACCAGGG | |
| | ACGAGAACGAGAAGCTGGAATATTACGAGAAGTTCCAGATCATCGAGAACGTGTTCAAG | |
| | CAGAAGAAGAAGCCCACCCTGAAGCAGATCGCCAAAGAAATCCTCGTGAACGAAGAGGA | |
| | TATTAAGGGCTACAGAGTGACCAGCACCGGCAAGCCCGAGTTCACCAACCTGAAGGTGT | |
| | ACCACGACATCAAGGACATTACCGCCCGGAAAGAGATTATTGAGAACGCCGAGCTGCTG | |
| | GATCAGATTGCCAAGATCCTGACCATCTACCAGAGCAGCGAGGACATCCAGGAAGAACT | |
| | GACCAATCTGAACTCCGAGCTGACCCAGGAAGAGATCGAGCAGATCTCTAATCTGAAGG | |
| | GCTATACCGGCACCCACAACCTGAGCCTGAAGGCCATCAACCTGATCCTGGACGAGCTG | |
| | TGGCACACCAACGACAACCAGATCGCTATCTTCAACCGGCTGAAGCTGGTGCCCAAGAA | |
| | GGTGGACCTGTCCCAGCAGAAAGAGATCCCCACCACCCTGGTGGACGACTTCATCCTGA | |
| | GCCCCGTCGTGAAGAGAAGCTTCATCCAGAGCATCAAAGTGATCAACGCCATCATCAAG | |
| | AAGTACGGCCTGCCCAACGACATCATTATCGAGCTGGCCCGCGAGAAGAACTCCAAGGA | |
| | CGCCCAGAAAATGATCAACGAGATGCAGAAGCGGAACCGGCAGACCAACGAGCGGATCG | |
| | AGGAAATCATCCGGACCACCGGCAAAGAGAACGCCAAGTACCTGATCGAGAAGATCAAG | |
| | CTGCACGACATGCAGGAAGGCAAGTGCCTGTACAGCCTGGAAGCCATCCCTCTGGAAGA | |
| | TCTGCTGAACAACCCCTTCAACTATGAGGTGGACCACATCATCCCCAGAAGCGTGTCCT | |
| | TCGACAACAGCTTCAACAACAAGGTGCTCGTGAAGCAGGAAGAAgcCAGCAAGAAGGGC | |
| | AACCGGACCCCATTCCAGTACCTGAGCAGCAGCGACAGCAAGATCAGCTACGAAACCTT | |
| | CAAGAAGCACATCCTGAATCTGGCCAAGGGCAAGGGCAGAATCAGCAAGACCAAGAAAG | |
| | AGTATCTGCTGGAAGAACGGGACATCAACAGGTTCTCCGTGCAGAAAGACTTCATCAAC | |
| | CGGAACCTGGTGGATACCAGATACGCCACCAGAGGCCTGATGAACCTGCTGCGGAGCTA | |
| | CTTCAGAGTGAACAACCTGGACGTGAAAGTGAAGTCCATCAATGGCGGCTTCACCAGCT | |
| | TTCTGCGGCGGAAGTGGAAGTTTAAGAAAGAGCGGAACAAGGGGTACAAGCACCACGCC | |
| | GAGGACGCCCTGATCATTGCCAACGCCGATTTCATCTTCAAAGAGTGGAAGAAACTGGA | |
| | CAAGGCCAAAAAAGTGATGGAAAACCAGATGTTCGAGGAAAAGCAGGCCGAGAGCATGC | |
| | CCGAGATCGAAACCGAGCAGGAGTACAAAGAGATCTTCATCACCCCCCACCAGATCAAG | |
| | CACATTAAGGACTTCAAGGACTACAAGTACAGCCACCGGGTGGACAAGAAGCCTAATAG | |
| | AGAGCTGATTAACGACACCCTGTACTCCACCCGGAAGGACGACAAGGGCAACACCCTGA | |
| | TCGTGAACAATCTGAACGGCCTGTACGACAAGGACAATGACAAGCTGAAAAAGCTGATC | |
| | AACAAGAGCCCCGAAAAGCTGCTGATGTACCACCACGACCCCCAGACCTACCAGAAACT | |
| | GAAGCTGATTATGGAACAGTACGGCGACGAGAAGAATCCCCTGTACAAGTACTACGAGG | |
| | AAACCGGGAACTACCTGACCAAGTACTCCAAAAAGGACAACGGCCCCGTGATCAAGAAG | |
| | ATTAAGTATTACGGCAACAAACTGAACGCCCATCTGGACATCACCGACGACTACCCCAA | |
| | CAGCAGAAACAAGGTCGTGAAGCTGTCCCTGAAGCCCTACAGATTCGACGTGTACCTGG | |
| | ACAATGGCGTGTACAAGTTCGTGACCGTGAAGAATCTGGATGTGATCAAAAAAGAAAAC | |
| | TACTACGAAGTGAATAGCAAGTGCTATGAGGAAGCTAAGAAGCTGAAGAAGATCAGCAA | |
| | CCAGGCCGAGTTTATCGCCTCCTTCTACAACAACGATCTGATCAAGATCAACGGCGAGC | |
| | TGTATAGAGTGATCGGCGTGAACAACGACCTGCTGAACCGGATCGAAGTGAACATGATC | |
| | GACATCACCTACCGCGAGTACCTGGAAAACATGAACGACAAGAGGCCCCCCAGGATCAT | |
| | TAAGACAATCGCCTCCAAGACCCAGAGCATTAAGAAGTACAGCACAGACATTCTGGGCA | |
| | ACCTGTATGAAGTGAAATCTAAGAAGCACCCTCAGATCATCAAAAAGGGCAAAAGGCCG | |
| | GCGGCCACGAAAAAGGCCGGCCAGGCAAAAAAGAAAAAGGctagCcgcgccgacgcgct | |
| | ggacgatttcgatctcgacatgctgggttctgatgccctcgatgactttgacctggata | |
| | tgttgggaagcgacgcattggatgactttgatctggacatgctcggctccgatgctctg | |
| | gacgatttcgatctcgatatgtta | |
| 70 | AAGCGGAACTACATCCTGGGCCTGGCCATCGGCATCACCAGCGTGGGCTACGGCATCAT | dSaCas9 (nt) |
| | CGACTACGAGACACGGGACGTGATCGATGCCGGCGTGCGGCTGTTCAAAGAGGCCAACG | |
| | TGGAAAACAACGAGGGCAGGCGGAGCAAGAGAGGCGCCAGAAGGCTGAAGCGGCGGAGG | |
| | CGGCATAGAATCCAGAGAGTGAAGAAGCTGCTGTTCGACTACAACCTGCTGACCGACCA | |
| | CAGCGAGCTGAGCGGCATCAACCCCTACGAGGCCAGAGTGAAGGGCCTGAGCCAGAAGC | |
| | TGAGCGAGGAAGAGTTCTCTGCCGCCCTGCTGCACCTGGCCAAGAGAAGAGGCGTGCAC | |
| | AACGTGAACGAGGTGGAAGAGGACACCGGCAACGAGCTGTCCACCAAAGAGCAGATCAG | |
| | CCGGAACAGCAAGGCCCTGGAAGAGAAATACGTGGCCGAACTGCAGCTGGAACGGCTGA | |
| | AGAAAGACGGCGAAGTGCGGGGCAGCATCAACAGATTCAAGACCAGCGACTACGTGAAA | |
| | GAAGCCAAACAGCTGCTGAAGGTGCAGAAGGCCTACCACCAGCTGGACCAGAGCTTCAT | |
| | CGACACCTACATCGACCTGCTGGAAACCCGGCGGACCTACTATGAGGGACCTGGCGAGG | |
| | GCAGCCCCTTCGGCTGGAAGGACATCAAAGAATGGTACGAGATGCTGATGGGCCACTGC | |
| | ACCTACTTCCCCGAGGAACTGCGGAGCGTGAAGTACGCCTACAACGCCGACCTGTACAA | |
| | CGCCCTGAACGACCTGAACAATCTCGTGATCACCAGGGACGAGAACGAGAAGCTGGAAT | |
| | ATTACGAGAAGTTCCAGATCATCGAGAACGTGTTCAAGCAGAAGAAGAAGCCCACCCTG | |
| | AAGCAGATCGCCAAAGAAATCCTCGTGAACGAAGAGGATATTAAGGGCTACAGAGTGAC | |
| | CAGCACCGGCAAGCCCGAGTTCACCAACCTGAAGGTGTACCACGACATCAAGGACATTA | |
| | CCGCCCGGAAAGAGATTATTGAGAACGCCGAGCTGCTGGATCAGATTGCCAAGATCCTG | |
| | ACCATCTACCAGAGCAGCGAGGACATCCAGGAAGAACTGACCAATCTGAACTCCGAGCT | |
| | GACCCAGGAAGAGATCGAGCAGATCTCTAATCTGAAGGGCTATACCGGCACCCACAACC | |
| | TGAGCCTGAAGGCCATCAACCTGATCCTGGACGAGCTGTGGCACACCAACGACAACCAG | |
| | ATCGCTATCTTCAACCGGCTGAAGCTGGTGCCCAAGAAGGTGGACCTGTCCCAGCAGAA | |
| | AGAGATCCCCACCACCCTGGTGGACGACTTCATCCTGAGCCCCGTCGTGAAGAGAAGCT | |
| | TCATCCAGAGCATCAAAGTGATCAACGCCATCATCAAGAAGTACGGCCTGCCCAACGAC | |
| | ATCATTATCGAGCTGGCCCGCGAGAAGAACTCCAAGGACGCCCAGAAAATGATCAACGA | |
| | GATGCAGAAGCGGAACCGGCAGACCAACGAGCGGATCGAGGAAATCATCCGGACCACCG | |
| | GCAAAGAGAACGCCAAGTACCTGATCGAGAAGATCAAGCTGCACGACATGCAGGAAGGC | |
| | AAGTGCCTGTACAGCCTGGAAGCCATCCCTCTGGAAGATCTGCTGAACAACCCCTTCAA | |
| | CTATGAGGTGGACCACATCATCCCCAGAAGCGTGTCCTTCGACAACAGCTTCAACAACA | |
| | AGGTGCTCGTGAAGCAGGAAGAAgcCAGCAAGAAGGGCAACCGGACCCCATTCCAGTAC | |
| | CTGAGCAGCAGCGACAGCAAGATCAGCTACGAAACCTTCAAGAAGCACATCCTGAATCT | |
| | GGCCAAGGGCAAGGGCAGAATCAGCAAGACCAAGAAAGAGTATCTGCTGGAAGAACGGG | |
| | ACATCAACAGGTTCTCCGTGCAGAAAGACTTCATCAACCGGAACCTGGTGGATACCAGA | |
| | TACGCCACCAGAGGCCTGATGAACCTGCTGCGGAGCTACTTCAGAGTGAACAACCTGGA | |
| | CGTGAAAGTGAAGTCCATCAATGGCGGCTTCACCAGCTTTCTGCGGCGGAAGTGGAAGT | |
| | TTAAGAAAGAGCGGAACAAGGGGTACAAGCACCACGCCGAGGACGCCCTGATCATTGCC | |
| | AACGCCGATTTCATCTTCAAAGAGTGGAAGAAACTGGACAAGGCCAAAAAAGTGATGGA | |
| | AAACCAGATGTTCGAGGAAAAGCAGGCCGAGAGCATGCCCGAGATCGAAACCGAGCAGG | |
| | AGTACAAAGAGATCTTCATCACCCCCCACCAGATCAAGCACATTAAGGACTTCAAGGAC | |
| | TACAAGTACAGCCACCGGGTGGACAAGAAGCCTAATAGAGAGCTGATTAACGACACCCT | |
| | GTACTCCACCCGGAAGGACGACAAGGGCAACACCCTGATCGTGAACAATCTGAACGGCC | |
| | TGTACGACAAGGACAATGACAAGCTGAAAAAGCTGATCAACAAGAGCCCCGAAAAGCTG | |
| | CTGATGTACCACCACGACCCCCAGACCTACCAGAAACTGAAGCTGATTATGGAACAGTA | |
| | CGGCGACGAGAAGAATCCCCTGTACAAGTACTACGAGGAAACCGGGAACTACCTGACCA | |
| | AGTACTCCAAAAAGGACAACGGCCCCGTGATCAAGAAGATTAAGTATTACGGCAACAAA | |
| | CTGAACGCCCATCTGGACATCACCGACGACTACCCCAACAGCAGAAACAAGGTCGTGAA | |
| | GCTGTCCCTGAAGCCCTACAGATTCGACGTGTACCTGGACAATGGCGTGTACAAGTTCG | |
| | TGACCGTGAAGAATCTGGATGTGATCAAAAAAGAAAACTACTACGAAGTGAATAGCAAG | |
| | TGCTATGAGGAAGCTAAGAAGCTGAAGAAGATCAGCAACCAGGCCGAGTTTATCGCCTC | |
| | CTTCTACAACAACGATCTGATCAAGATCAACGGCGAGCTGTATAGAGTGATCGGCGTGA | |
| | ACAACGACCTGCTGAACCGGATCGAAGTGAACATGATCGACATCACCTACCGCGAGTAC | |
| | CTGGAAAACATGAACGACAAGAGGCCCCCCAGGATCATTAAGACAATCGCCTCCAAGAC | |
| | CCAGAGCATTAAGAAGTACAGCACAGACATTCTGGGCAACCTGTATGAAGTGAAATCTA | |
| | AGAAGCACCCTCAGATCATCAAAAAGGGC | |
| 71 | DALDDFDLDMLGSDALDDFDLDMLGSDALDDFDLDMLGSDALDDFDLDMVNPKKKRKVG | VP64- |
| | IHGVPAAKRNYILGLAIGITSVGYGIIDYETRDVIDAGVRLFKEANVENNEGRRSKRGA | dSaCas9-NLS- |
| | RRLKRRRRHRIQRVKKLLEDYNLLTDHSELSGINPYEARVKGLSQKLSEEEFSAALLHL | VP64 (aa) |
| | AKRRGVHNVNEVEEDTGNELSTKEQISRNSKALEEKYVAELQLERLKKDGEVRGSINRF | |
| | KTSDYVKEAKQLLKVQKAYHQLDQSFIDTYIDLLETRRTYYEGPGEGSPFGWKDIKEWY | |
| | EMLMGHCTYFPEELRSVKYAYNADLYNALNDLNNLVITRDENEKLEYYEKFQIIENVEK | |
| | QKKKPTLKQIAKEILVNEEDIKGYRVTSTGKPEFTNLKVYHDIKDITARKEIIENAELL | |
| | DQIAKILTIYQSSEDIQEELTNLNSELTQEEIEQISNLKGYTGTHNLSLKAINLILDEL | |
| | WHTNDNQIAIFNRLKLVPKKVDLSQQKEIPTTLVDDFILSPVVKRSFIQSIKVINAIIK | |
| | KYGLPNDIIIELAREKNSKDAQKMINEMQKRNRQTNERIEEIIRTTGKENAKYLIEKIK | |
| | LHDMQEGKCLYSLEAIPLEDLLNNPFNYEVDHIIPRSVSFDNSFNNKVLVKQEEASKKG | |
| | NRTPFQYLSSSDSKISYETFKKHILNLAKGKGRISKTKKEYLLEERDINRFSVQKDFIN | |
| | RNLVDTRYATRGLMNLLRSYFRVNNLDVKVKSINGGFTSFLRRKWKFKKERNKGYKHHA | |
| | EDALIIANADFIFKEWKKLDKAKKVMENQMFEEKQAESMPEIETEQEYKEIFITPHQIK | |
| | HIKDFKDYKYSHRVDKKPNRELINDTLYSTRKDDKGNTLIVNNLNGLYDKDNDKLKKLI | |
| | NKSPEKLLMYHHDPQTYQKLKLIMEQYGDEKNPLYKYYEETGNYLTKYSKKDNGPVIKK | |
| | IKYYGNKLNAHLDITDDYPNSRNKVVKLSLKPYRFDVYLDNGVYKFVTVKNLDVIKKEN | |
| | YYEVNSKCYEEAKKLKKISNQAEFIASFYNNDLIKINGELYRVIGVNNDLLNRIEVNMI | |
| | DITYREYLENMNDKRPPRIIKTIASKTQSIKKYSTDILGNLYEVKSKKHPQIIKKGKRP | |
| | AATKKAGQAKKKKASRADALDDFDLDMLGSDALDDFDLDMLGSDALDDFDLDMLGSDAL | |
| | DDFDLDML | |
| 72 | KRNYILGLAIGITSVGYGIIDYETRDVIDAGVRLFKEANVENNEGRRSKRGARRLKRRR | dSaCas9 (aa) |
| | RHRIQRVKKLLFDYNLLTDHSELSGINPYEARVKGLSQKLSEEEFSAALLHLAKRRGVH | |
| | NVNEVEEDTGNELSTKEQISRNSKALEEKYVAELQLERLKKDGEVRGSINRFKTSDYVK | |
| | EAKQLLKVQKAYHQLDQSFIDTYIDLLETRRTYYEGPGEGSPFGWKDIKEWYEMLMGHC | |
| | TYFPEELRSVKYAYNADLYNALNDLNNLVITRDENEKLEYYEKFQIIENVFKQKKKPTL | |
| | KQIAKEILVNEEDIKGYRVTSTGKPEFTNLKVYHDIKDITARKEIIENAELLDQIAKIL | |
| | TIYQSSEDIQEELTNLNSELTQEEIEQISNLKGYTGTHNLSLKAINLILDELWHINDNQ | |
| | IAIFNRLKLVPKKVDLSQQKEIPTTLVDDFILSPVVKRSFIQSIKVINAIIKKYGLPND | |
| | IIIELAREKNSKDAQKMINEMQKRNRQTNERIEEIIRTTGKENAKYLIEKIKLHDMQEG | |
| | KCLYSLEAIPLEDLLNNPFNYEVDHIIPRSVSFDNSFNNKVLVKQEEASKKGNRTPFQY | |
| | LSSSDSKISYETFKKHILNLAKGKGRISKTKKEYLLEERDINRFSVQKDFINRNLVDTR | |
| | YATRGLMNLLRSYFRVNNLDVKVKSINGGFTSFLRRKWKFKKERNKGYKHHAEDALIIA | |
| | NADFIFKEWKKLDKAKKVMENQMFEEKQAESMPEIETEQEYKEIFITPHQIKHIKDFKD | |
| | YKYSHRVDKKPNRELINDTLYSTRKDDKGNTLIVNNLNGLYDKDNDKLKKLINKSPEKL | |
| | LMYHHDPQTYQKLKLIMEQYGDEKNPLYKYYEETGNYLTKYSKKDNGPVIKKIKYYGNK | |
| | LNAHLDITDDYPNSRNKVVKLSLKPYRFDVYLDNGVYKFVTVKNLDVIKKENYYEVNSK | |
| | CYEEAKKLKKISNQAEFIASFYNNDLIKINGELYRVIGVNNDLLNRIEVNMIDITYREY | |
| | LENMNDKRPPRIIKTIASKTQSIKKYSTDILGNLYEVKSKKHPQIIKKG | |
| 73 | MKRNYILGLDIGITSVGYGIIDYETRDVIDAGVRLFKEANVENNEGRRSKRGARRLKRR | WT Sa Cas9 |
| | RRHRIQRVKKLLFDYNLLTDHSELSGINPYEARVKGLSQKLSEEEFSAALLHLAKRRGV | (aa) |
| | HNVNEVEEDTGNELSTKEQISRNSKALEEKYVAELQLERLKKDGEVRGSINRFKTSDYV | |
| | KEAKQLLKVQKAYHQLDQSFIDTYIDLLETRRTYYEGPGEGSPFGWKDIKEWYEMLMGH | |
| | CTYFPEELRSVKYAYNADLYNALNDLNNLVITRDENEKLEYYEKFQIIENVFKQKKKPT | |
| | LKQIAKEILVNEEDIKGYRVTSTGKPEFTNLKVYHDIKDITARKEIIENAELLDQIAKI | |
| | LTIYQSSEDIQEELTNLNSELTQEEIEQISNLKGYTGTHNLSLKAINLILDELWHINDN | |
| | QIAIFNRLKLVPKKVDLSQQKEIPTTLVDDFILSPVVKRSFIQSIKVINAIIKKYGLPN | |
| | DIIIELAREKNSKDAQKMINEMQKRNRQTNERIEEIIRTTGKENAKYLIEKIKLHDMQE | |
| | GKCLYSLEAIPLEDLLNNPFNYEVDHIIPRSVSFDNSFNNKVLVKQEENSKKGNRTPFQ | |
| | YLSSSDSKISYETFKKHILNLAKGKGRISKTKKEYLLEERDINRFSVQKDFINRNLVDT | |
| | RYATRGLMNLLRSYFRVNNLDVKVKSINGGFTSFLRRKWKFKKERNKGYKHHAEDALII | |
| | ANADFIFKEWKKLDKAKKVMENQMFEEKQAESMPEIETEQEYKEIFITPHQIKHIKDFK | |
| | DYKYSHRVDKKPNRELINDTLYSTRKDDKGNTLIVNNLNGLYDKDNDKLKKLINKSPEK | |
| | LLMYHHDPQTYQKLKLIMEQYGDEKNPLYKYYEETGNYLTKYSKKDNGPVIKKIKYYGN | |
| | KLNAHLDITDDYPNSRNKVVKLSLKPYRFDVYLDNGVYKFVTVKNLDVIKKENYYEVNS | |
| | KCYEEAKKLKKISNQAEFIASFYNNDLIKINGELYRVIGVNNDLLNRIEVNMIDITYRE | |
| | YLENMNDKRPPRIIKTIASKTQSIKKYSTDILGNLYEVKSKKHPQIIKKG | |
| 74 | GACATTGATTATTGACTAGTTATTAATAGTAATCAATTACGGGGTCATTAGTTCATAGC | gRNA VP64- |
| | CCATATATGGAGTTCCGCGTTACATAACTTACGGTAAATGGCCCGCCTGGCTGACCGCC | dSpCas9-NLS- |
| | CAACGACCCCCGCCCATTGACGTCAATAATGACGTATGTTCCCATAGTAACGCCAATAG | VP64 vector |
| | GGACTTTCCATTGACGTCAATGGGTGGAGTATTTACGGTAAACTGCCCACTTGGCAGTA | |
| | CATCAAGTGTATCATATGCCAAGTACGCCCCCTATTGACGTCAATGACGGTAAATGGCC | |
| | CGCCTGGCATTATGCCCAGTACATGACCTTATGGGACTTTCCTACTTGGCAGTACATCT | |
| | ACGTATTAGTCATCGCTATTACCATGGTGATGCGGTTTTGGCAGTACATCAATGGGCGT | |
| | GGATAGCGGTTTGACTCACGGGGATTTCCAAGTCTCCACCCCATTGACGTCAATGGGAG | |
| | TTTGTTTTGGCACCAAAATCAACGGGACTTTCCAAAATGTCGTAACAACTCCGCCCCAT | |
| | TGACGCAAATGGGCGGTAGGCGTGTACGGTGGGAGGTCTATATAAGCAGCGCGTTTTGC | |
| | CTGTACTGGGTCTCTCTGGTTAGACCAGATCTGAGCCTGGGAGCTCTCTGGCTAACTAG | |
| | GGAACCCACTGCTTAAGCCTCAATAAAGCTTGCCTTGAGTGCTTCAAGTAGTGTGTGCC | |
| | CGTCTGTTGTGTGACTCTGGTAACTAGAGATCCCTCAGACCCTTTTAGTCAGTGTGGAA | |
| | AATCTCTAGCAGTGGCGCCCGAACAGGGACTTGAAAGCGAAAGGGAAACCAGAGGAGCT | |
| | CTCTCGACGCAGGACTCGGCTTGCTGAAGCGCGCACGGCAAGAGGCGAGGGGCGGCGAC | |
| | TGGTGAGTACGCCAAAAATTTTGACTAGCGGAGGCTAGAAGGAGAGAGATGGGTGCGAG | |
| | AGCGTCAGTATTAAGCGGGGGAGAATTAGATCGCGATGGGAAAAAATTCGGTTAAGGCC | |
| | AGGGGGAAAGAAAAAATATAAATTAAAACATATAGTATGGGCAAGCAGGGAGCTAGAAC | |
| | GATTCGCAGTTAATCCTGGCCTGTTAGAAACATCAGAAGGCTGTAGACAAATACTGGGA | |
| | CAGCTACAACCATCCCTTCAGACAGGATCAGAAGAACTTAGATCATTATATAATACAGT | |
| | AGCAACCCTCTATTGTGTGCATCAAAGGATAGAGATAAAAGACACCAAGGAAGCTTTAG | |
| | ACAAGATAGAGGAAGAGCAAAACAAAAGTAAGACCACCGCACAGCAAGCGGCCGCTGAT | |
| | CTTCAGACCTGGAGGAGGAGATATGAGGGACAATTGGAGAAGTGAATTATATAAATATA | |
| | AAGTAGTAAAAATTGAACCATTAGGAGTAGCACCCACCAAGGCAAAGAGAAGAGTGGTG | |
| | CAGAGAGAAAAAAGAGCAGTGGGAATAGGAGCTTTGTTCCTTGGGTTCTTGGGAGCAGC | |
| | AGGAAGCACTATGGGCGCAGCGTCAATGACGCTGACGGTACAGGCCAGACAATTATTGT | |
| | CTGGTATAGTGCAGCAGCAGAACAATTTGCTGAGGGCTATTGAGGCGCAACAGCATCTG | |
| | TTGCAACTCACAGTCTGGGGCATCAAGCAGCTCCAGGCAAGAATCCTGGCTGTGGAAAG | |
| | ATACCTAAAGGATCAACAGCTCCTGGGGATTTGGGGTTGCTCTGGAAAACTCATTTGCA | |
| | CCACTGCTGTGCCTTGGAATGCTAGTTGGAGTAATAAATCTCTGGAACAGATTTGGAAT | |
| | CACACGACCTGGATGGAGTGGGACAGAGAAATTAACAATTACACAAGCTTAATACACTC | |
| | CTTAATTGAAGAATCGCAAAACCAGCAAGAAAAGAATGAACAAGAATTATTGGAATTAG | |
| | ATAAATGGGCAAGTTTGTGGAATTGGTTTAACATAACAAATTGGCTGTGGTATATAAAA | |
| | TTATTCATAATGATAGTAGGAGGCTTGGTAGGTTTAAGAATAGTTTTTGCTGTACTTTC | |
| | TATAGTGAATAGAGTTAGGCAGGGATATTCACCATTATCGTTTCAGACCCACCTCCCAA | |
| | CCCCGAGGGGACCCGACAGGCCCGAAGGAATAGAAGAAGAAGGTGGAGAGAGAGACAGA | |
| | GACAGATCCATTCGATTAGTGAACGGATCGGCACTGCGTGCGCCAATTCTGCAGACAAA | |
| | TGGCAGTATTCATCCACAATTTTAAAAGAAAAGGGGGGATTGGGGGGTACAGTGCAGGG | |
| | GAAAGAATAGTAGACATAATAGCAACAGACATACAAACTAAAGAATTACAAAAACAAAT | |
| | TACAAAAATTCAAAATTTTCGGGTTTATTACAGGGACAGCAGAGATCCAGTTTGGTTAA | |
| | TTAAAAAAAAAGCACCGACTCGGTGCCACTTTTTCAAGTTGATAACGGACTAGCCTTAT | |
| | TTAAACTTGCTATGCTGTTTCCAGCATAGCTCTTAAACAGAGACGATTTACAACCGTCT | |
| | CCGGTGTTTCGTCCTTTCCACAAGATATATAAAGCCAAGAAATCGAAATACTTTCAAGT | |
| | TACGGTAAGCATATGATAGTCCATTTTAAAACATAATTTTAAAACTGCAAACTACCCAA | |
| | GAAATTATTACTTTCTACGTCACGTATTTTGTACTAATATCTTTGTGTTTACAGTCAAA | |
| | TTAATTCCAATTATCTCTCTAACAGCCTTGTATCGTATATGCAAATATGAAGGAATCAT | |
| | GGGAAATAGGCCCTCTCTAGAGATATCCCTAGCGGCCTCCGCGCCGGGTTTTGGCGCCT | |
| | CCCGCGGGCGCCCCCCTCCTCACGGCGAGCGCTGCCACGTCAGACGAAGGGCGCAGCGA | |
| | GCGTCCTGATCCTTCCGCCCGGACGCTCAGGACAGCGGCCCGCTGCTCATAAGACTCGG | |
| | CCTTAGAACCCCAGTATCAGCAGAAGGACATTTTAGGACGGGACTTGGGTGACTCTAGG | |
| | GCACTGGTTTTCTTTCCAGAGAGCGGAACAGGCGAGGAAAAGTAGTCCCTTCTCGGCGA | |
| | TTCTGCGGAGGGATCTCCGTGGGGCGGTGAACGCCGATGATTATATAAGGACGCGCCGG | |
| | GTGTGGCACAGCTAGTTCCGTCGCAGCCGGGATTTGGGTCGCGGTTCTTGTTTGTGGAT | |
| | CGCTGTGATCGTCACTTGGTGAGTAGCGGGCTGCTGGGCTGGCCGGGGCTTTCGTGGCC | |
| | GCCGGGCCGCTCGGTGGGACGGAAGCGTGTGGAGAGACCGCCAAGGGCTGTAGTCTGGG | |
| | TCCGCGAGCAAGGTTGCCCTGAACTGGGGGTTGGGGGGAGCGCAGCAAAATGGCGGCTG | |
| | TTCCCGAGTCTTGAATGGAAGACGCTTGTGAGGCGGGCTGTGAGGTCGTTGAAACAAGG | |
| | TGGGGGGCATGGTGGGCGGCAAGAACCCAAGGTCTTGAGGCCTTCGCTAATGCGGGAAA | |
| | GCTCTTATTCGGGTGAGATGGGCTGGGGCACCATCTGGGGACCCTGACGTGAAGTTTGT | |
| | CACTGACTGGAGAACTCGGTTTGTCGTCTGTTGCGGGGGCGGCAGTTATGGCGGTGCCG | |
| | TTGGGCAGTGCACCCGTACCTTTGGGAGCGCGCGCCCTCGTCGTGTCGTGACGTCACCC | |
| | GTTCTGTTGGCTTATAATGCAGGGTGGGGCCACCTGCCGGTAGGTGTGCGGTAGGCTTT | |
| | TCTCCGTCGCAGGACGCAGGGTTCGGGCCTAGGGTAGGCTCTCCTGAATCGACAGGCGC | |
| | CGGACCTCTGGTGAGGGGAGGGATAAGTGAGGCGTCAGTTTCTTTGGTCGGTTTTATGT | |
| | ACCTATCTTCTTAAGTAGCTGAAGCTCCGGTTTTGAACTATGCGCTCGGGGTTGGCGAG | |
| | TGTGTTTTGTGAAGTTTTTTAGGCACCTTTTGAAATGTAATCATTTGGGTCAATATGTA | |
| | ATTTTCAGTGTTAGACTAGTAAATTGTCCGCTAAATTCTGGCCGTTTTTGGCTTTTTTG | |
| | TTAGACGACCGGTTCTAGCGCGCTGCCACCATGGACTACAAAGACCATGACGGTGATTA | |
| | TAAAGATCATGACATCGATTACAAGGATGACGATGACAAGCACggtccgcgggctgacg | |
| | cattggacgattttgatctggatatgctgggaagtgacgccctcgatgattttgacctt | |
| | gacatgcttggttcggatgcccttgatgactttgacctcgacatgctcggcagtgacgc | |
| | ccttgatgatttcgacctggacatgGTTAACCCAAAGAAGAAGCGGAAGGTCGGTATCC | |
| | ACGGAGTCCCAGCAGCCGACAAGAAGTACTCCATTGGGCTCGCCATCGGCACAAACAGC | |
| | GTCGGCTGGGCCGTCATTACGGACGAGTACAAGGTGCCGAGCAAAAAATTCAAAGTTCT | |
| | GGGCAATACCGATCGCCACAGCATAAAGAAGAACCTCATTGGCGCCCTCCTGTTCGACT | |
| | CCGGGGAAACCGCCGAAGCCACGCGGCTCAAAAGAACAGCACGGCGCAGATATACCCGC | |
| | AGAAAGAATCGGATCTGCTACCtgcaGGAGATCTTTAGTAATGAGATGGCTAAGGTGGA | |
| | TGACTCTTTCTTCCATAGGCTGGAGGAGTCCTTTTTGGTGGAGGAGGATAAAAAGCACG | |
| | AGCGCCACCCAATCTTTGGCAATATCGTGGACGAGGTGGCGTACCATGAAAAGTACCCA | |
| | ACCATATATCATCTGAGGAAGAAGCTTGTAGACAGTACTGATAAGGCTGACTTGCGGTT | |
| | GATCTATCTCGCGCTGGCGCATATGATCAAATTTCGGGGACACTTCCTCATCGAGGGGG | |
| | ACCTGAACCCAGACAACAGCGATGTCGACAAACTCTTTATCCAACTGGTTCAGACTTAC | |
| | AATCAGCTTTTCGAAGAGAACCCGATCAACGCATCCGGAGTTGACGCCAAAGCAATCCT | |
| | GAGCGCTAGGCTGTCCAAATCCCGGCGGCTCGAAAACCTCATCGCACAGCTCCCTGGGG | |
| | AGAAGAAGAACGGCCTGTTTGGTAATCTTATCGCCCTGTCACTCGGGCTGACCCCCAAC | |
| | TTTAAATCTAACTTCGACCTGGCCGAAGATGCCAAGCTTCAACTGAGCAAAGACACCTA | |
| | CGATGATGATCTCGACAATCTGCTGGCCCAGATCGGCGACCAGTACGCAGACCTTTTTT | |
| | TGGCGGCAAAGAACCTGTCAGACGCCATTCTGCTGAGTGATATTCTGCGAGTGAACACG | |
| | GAGATCACCAAAGCTCCGCTGAGCGCTAGTATGATCAAGCGCTATGATGAGCACCACCA | |
| | AGACTTGACTTTGCTGAAGGCCCTTGTCAGACAGCAACTGCCTGAGAAGTACAAGGAAA | |
| | TTTTCTTCGATCAGTCTAAAAATGGCTACGCCGGATACATTGACGGCGGAGCAAGCCAG | |
| | GAGGAATTTTACAAATTTATTAAGCCCATCTTGGAAAAAATGGACGGCACCGAGGAGCT | |
| | GCTGGTAAAGCTTAACAGAGAAGATCTGTTGCGCAAACAGCGCACTTTCGACAATGGAA | |
| | GCATCCCCCACCAGATTCACCTGGGCGAACTGCACGCTATCCTCAGGCGGCAAGAGGAT | |
| | TTCTACCCCTTTTTGAAAGATAACAGGGAAAAGATTGAGAAAATCCTCACATTTCGGAT | |
| | ACCCTACTATGTAGGCCCCCTCGCCCGGGGAAATTCCAGATTCGCGTGGATGACTCGCA | |
| | AATCAGAAGAGACCATCACTCCCTGGAACTTCGAGGAAGTCGTGGATAAGGGGGCCTCT | |
| | GCCCAGTCCTTCATCGAAAGGATGACTAACTTTGATAAAAATCTGCCTAACGAAAAGGT | |
| | GCTTCCTAAACACTCTCTGCTGTACGAGTACTTCACAGTTTATAACGAGCTCACCAAGG | |
| | TCAAATACGTCACAGAAGGGATGAGAAAGCCAGCATTCCTGTCTGGAGAGCAGAAGAAA | |
| | GCTATCGTGGACCTCCTCTTCAAGACGAACCGGAAAGTTACCGTGAAACAGCTCAAAGA | |
| | AGACTATTTCAAAAAGATTGAATGTTTCGACTCTGTTGAAATCAGCGGAGTGGAGGATC | |
| | GCTTCAACGCATCCCTGGGAACGTATCACGATCTCCTGAAAATCATTAAAGACAAGGAC | |
| | TTCCTGGACAATGAGGAGAACGAGGACATTCTTGAGGACATTGTCCTCACCCTTACGTT | |
| | GTTTGAAGATAGGGAGATGATTGAAGAACGCTTGAAAACTTACGCTCATCTCTTCGACG | |
| | ACAAAGTCATGAAACAGCTCAAGAGGCGCCGATATACAGGATGGGGGCGGCTGTCAAGA | |
| | AAACTGATCAATGGgatcCGAGACAAGCAGAGTGGAAAGACAATCCTGGATTTTCTTAA | |
| | GTCCGATGGATTTGCCAACCGGAACTTCATGCAGTTGATCCATGATGACTCTCTCACCT | |
| | TTAAGGAGGACATCCAGAAAGCACAAGTTTCTGGCCAGGGGGACAGTCTTCACGAGCAC | |
| | ATCGCTAATCTTGCAGGTAGCCCAGCTATCAAAAAGGGAATACTGCAGACCGTTAAGGT | |
| | CGTGGATGAACTCGTCAAAGTAATGGGAAGGCATAAGCCCGAGAATATCGTTATCGAGA | |
| | TGGCCCGAGAGAACCAAACTACCCAGAAGGGACAGAAGAACAGTAGGGAAAGGATGAAG | |
| | AGGATTGAAGAGGGTATAAAAGAACTGGGGTCCCAAATCCTTAAGGAACACCCAGTTGA | |
| | AAACACCCAGCTTCAGAATGAGAAGCTCTACCTGTACTACCTGCAGAACGGCAGGGACA | |
| | TGTACGTGGATCAGGAACTGGACATCAATCGGCTCTCCGACTACGACGTGGATGCCATC | |
| | GTGCCCCAGTCTTTTCTCAAAGATGATTCTATTGATAATAAAGTGTTGACAAGATCCGA | |
| | TAAAAATAGAGGGAAGAGTGATAACGTCCCCTCAGAAGAAGTTGTCAAGAAAATGAAAA | |
| | ATTATTGGCGGCAGCTGCTGAACGCCAAACTGATCACACAACGGAAGTTCGATAATCTG | |
| | ACTAAGGCTGAACGAGGTGGCCTGTCTGAGTTGGATAAAGCCGGCTTCATCAAAAGGCA | |
| | GCTTGTTGAGACACGCCAGATCACCAAgcacGTGGCCCAAATTCTCGATTCACGCATGA | |
| | ACACCAAGTACGATGAAAATGACAAACTGATTCGAGAGGTGAAAGTTATTACTCTGAAG | |
| | TCTAAGCTGGTCTCAGATTTCAGAAAGGACTTTCAGTTTTATAAGGTGAGAGAGATCAA | |
| | CAATTACCACCATGCGCATGATGCCTACCTGAATGCAGTGGTAGGCACTGCACTTATCA | |
| | AAAAATATCCCAAGCTTGAATCTGAATTTGTTTACGGAGACTATAAAGTGTACGATGTT | |
| | AGGAAAATGATCGCAAAGTCTGAGCAGGAAATAGGCAAGGCCACCGCTAAGTACTTCTT | |
| | TTACAGCAATATTATGAATTTTTTCAAGACCGAGATTACACTGGCCAATGGAGAGATTC | |
| | GGAAGCGACCACTTATCGAAACAAACGGAGAAACAGGAGAAATCGTGTGGGACAAGGGT | |
| | AGGGATTTCGCGACAGTCCGGAAGGTCCTGTCCATGCCGCAGGTGAACATCGTTAAAAA | |
| | GACCGAAGTACAGACCGGAGGCTTCTCCAAGGAAAGTATCCTCCCGAAAAGGAACAGCG | |
| | ACAAGCTGATCGCACGCAAAAAAGATTGGGACCCCAAGAAATACGGCGGATTCGATTCT | |
| | CCTACAGTCGCTTACAGTGTACTGGTTGTGGCCAAAGTGGAGAAAGGGAAGTCTAAAAA | |
| | ACTCAAAAGCGTCAAGGAACTGCTGGGCATCACAATCATGGAGCGATCAAGCTTCGAAA | |
| | AAAACCCCATCGACTTTCTCGAGGCGAAAGGATATAAAGAGGTCAAAAAAGACCTCATC | |
| | ATTAAGCTTCCCAAGTACTCTCTCTTTGAGCTTGAAAACGGCCGGAAACGAATGCTCGC | |
| | TAGTGCGGGCGAGCTGCAGAAAGGTAACGAGCTGGCACTGCCCTCTAAATACGTTAATT | |
| | TCTTGTATCTGGCCAGCCACTATGAAAAGCTCAAAGGGTCTCCCGAAGATAATGAGCAG | |
| | AAGCAGCTGTTCGTGGAACAACACAAACACTACCTTGATGAGATCATCGAGCAAATAAG | |
| | CGAATTCTCCAAAAGAGTGATCCTCGCCGACGCTAACCTCGATAAGGTGCTTTCTGCTT | |
| | ACAATAAGCACAGGGATAAGCCCATCAGGGAGCAGGCAGAAAACATTATCCACTTGTTT | |
| | ACTCTGACCAACTTGGGCGCGCCTGCAGCCTTCAAGTACTTCGACACCACCATAGACAG | |
| | AAAGCGGTACACCTCTACAAAGGAGGTCCTGGACGCCACACTGATTCATCAGTCAATTA | |
| | CGGGGCTCTATGAAACAAGAATCGACCTCTCTCAGCTCGGTGGAGACAAAAGGCCGGCG | |
| | GCCACGAAAAAGGCCGGCCAGGCAAAAAAGAAAAAGGctagCcgcgccgacgcgctgga | |
| | cgatttcgatctcgacatgctgggttctgatgccctcgatgactttgacctggatatgt | |
| | tgggaagcgacgcattggatgactttgatctggacatgctcggctccgatgctctggac | |
| | gatttcgatctcgatatgttaatcgctagcGCAACAAACTTCTCTCTGCTGAAACAAGC | |
| | CGGAGATGTCGAAGAGAATCCTGGACCGATGACCGAGTACAAGCCCACGGTGCGCCTCG | |
| | CCACCCGCGACGACGTCCCCAGGGCCGTACGCACCCTCGCCGCCGCGTTCGCCGACTAC | |
| | CCCGCCACGCGCCACACCGTCGATCCGGACCGCCACATCGAGCGGGTCACCGAGCTGCA | |
| | AGAACTCTTCCTCACGCGCGTCGGGCTCGACATCGGCAAGGTGTGGGTCGCGGACGACG | |
| | GCGCCGCGGTGGCGGTCTGGACCACGCCGGAGAGCGTCGAAGCGGGGGCGGTGTTCGCC | |
| | GAGATCGGCCCGCGCATGGCCGAGTTGAGCGGTTCCCGGCTGGCCGCGCAGCAACAGAT | |
| | GGAAGGCCTCCTGGCGCCGCACCGGCCCAAGGAGCCCGCGTGGTTCCTGGCCACCGTCG | |
| | GCGTGTCGCCCGACCACCAGGGCAAGGGTCTGGGCAGCGCCGTCGTGCTCCCCGGAGTG | |
| | GAGGCGGCCGAGCGCGCCGGGGTGCCCGCCTTCCTGGAGACCTCCGCGCCCCGCAACCT | |
| | CCCCTTCTACGAGCGGCTCGGCTTCACCGTCACCGCCGACGTCGAGGTGCCCGAAGGAC | |
| | CGCGCACCTGGTGCATGACCCGCAAGCCCGGTGCCTGAATTTAAATGAATTCGATATCA | |
| | AGCTTATCGATAATCAACCTCTGGATTACAAAATTTGTGAAAGATTGACTGGTATTCTT | |
| | AACTATGTTGCTCCTTTTACGCTATGTGGATACGCTGCTTTAATGCCTTTGTATCATGC | |
| | TATTGCTTCCCGTATGGCTTTCATTTTCTCCTCCTTGTATAAATCCTGGTTGCTGTCTC | |
| | TTTATGAGGAGTTGTGGCCCGTTGTCAGGCAACGTGGCGTGGTGTGCACTGTGTTTGCT | |
| | GACGCAACCCCCACTGGTTGGGGCATTGCCACCACCTGTCAGCTCCTTTCCGGGACTTT | |
| | CGCTTTCCCCCTCCCTATTGCCACGGCGGAACTCATCGCCGCCTGCCTTGCCCGCTGCT | |
| | GGACAGGGGCTCGGCTGTTGGGCACTGACAATTCCGTGGTGTTGTCGGGGAAATCATCG | |
| | TCCTTTCCTTGGCTGCTCGCCTGTGTTGCCACCTGGATTCTGCGCGGGACGTCCTTCTG | |
| | CTACGTCCCTTCGGCCCTCAATCCAGCGGACCTTCCTTCCCGCGGCCTGCTGCCGGCTC | |
| | TGCGGCCTCTTCCGCGTCTTCGCCTTCGCCCTCAGACGAGTCGGATCTCCCTTTGGGCC | |
| | GCCTCCCCGCATCGATACCGTCGACCTCGAGACCTAGAAAAACATGGAGCAATCACAAG | |
| | TAGCAATACAGCAGCTACCAATGCTGATTGTGCCTGGCTAGAAGCACAAGAGGAGGAGG | |
| | AGGTGGGTTTTCCAGTCACACCTCAGGTACCTTTAAGACCAATGACTTACAAGGCAGCT | |
| | GTAGATCTTAGCCACTTTTTAAAAGAAAAGGGGGGACTGGAAGGGCTAATTCACTCCCA | |
| | ACGAAGACAAGATATCCTTGATCTGTGGATCTACCACACACAAGGCTACTTCCCTGATT | |
| | GGCAGAACTACACACCAGGGCCAGGGATCAGATATCCACTGACCTTTGGATGGTGCTAC | |
| | AAGCTAGTACCAGTTGAGCAAGAGAAGGTAGAAGAAGCCAATGAAGGAGAGAACACCCG | |
| | CTTGTTACACCCTGTGAGCCTGCATGGGATGGATGACCCGGAGAGAGAAGTATTAGAGT | |
| | GGAGGTTTGACAGCCGCCTAGCATTTCATCACATGGCCCGAGAGCTGCATCCGGACTGT | |
| | ACTGGGTCTCTCTGGTTAGACCAGATCTGAGCCTGGGAGCTCTCTGGCTAACTAGGGAA | |
| | CCCACTGCTTAAGCCTCAATAAAGCTTGCCTTGAGTGCTTCAAGTAGTGTGTGCCCGTC | |
| | TGTTGTGTGACTCTGGTAACTAGAGATCCCTCAGACCCTTTTAGTCAGTGTGGAAAATC | |
| | TCTAGCAGGGCCCGTTTAAACCCGCTGATCAGCCTCGACTGTGCCTTCTAGTTGCCAGC | |
| | CATCTGTTGTTTGCCCCTCCCCCGTGCCTTCCTTGACCCTGGAAGGTGCCACTCCCACT | |
| | GTCCTTTCCTAATAAAATGAGGAAATTGCATCGCATTGTCTGAGTAGGTGTCATTCTAT | |
| | TCTGGGGGGTGGGGTGGGGCAGGACAGCAAGGGGGAGGATTGGGAAGACAATAGCAGGC | |
| | ATGCTGGGGATGCGGTGGGCTCTATGGCTTCTGAGGCGGAAAGAACCAGCTGGGGCTCT | |
| | AGGGGGTATCCCCACGCGCCCTGTAGCGGCGCATTAAGCGCGGCGGGTGTGGTGGTTAC | |
| | GCGCAGCGTGACCGCTACACTTGCCAGCGCCCTAGCGCCCGCTCCTTTCGCTTTCTTCC | |
| | CTTCCTTTCTCGCCACGTTCGCCGGCTTTCCCCGTCAAGCTCTAAATCGGGGGCTCCCT | |
| | TTAGGGTTCCGATTTAGTGCTTTACGGCACCTCGACCCCAAAAAACTTGATTAGGGTGA | |
| | TGGTTCACGTAGTGGGCCATCGCCCTGATAGACGGTTTTTCGCCCTTTGACGTTGGAGT | |
| | CCACGTTCTTTAATAGTGGACTCTTGTTCCAAACTGGAACAACACTCAACCCTATCTCG | |
| | GTCTATTCTTTTGATTTATAAGGGATTTTGCCGATTTCGGCCTATTGGTTAAAAAATGA | |
| | GCTGATTTAACAAAAATTTAACGCGAATTAATTCTGTGGAATGTGTGTCAGTTAGGGTG | |
| | TGGAAAGTCCCCAGGCTCCCCAGCAGGCAGAAGTATGCAAAGCATGCATCTCAATTAGT | |
| | CAGCAACCAGGTGTGGAAAGTCCCCAGGCTCCCCAGCAGGCAGAAGTATGCAAAGCATG | |
| | CATCTCAATTAGTCAGCAACCATAGTCCCGCCCCTAACTCCGCCCATCCCGCCCCTAAC | |
| | TCCGCCCAGTTCCGCCCATTCTCCGCCCCATGGCTGACTAATTTTTTTTATTTATGCAG | |
| | AGGCCGAGGCCGCCTCTGCCTCTGAGCTATTCCAGAAGTAGTGAGGAGGCTTTTTTGGA | |
| | GGCCTAGGCTTTTGCAAAAAGCTCCCGGGAGCTTGTATATCCATTTTCGGATCTGATCA | |
| | GCACGTGTTGACAATTAATCATCGGCATAGTATATCGGCATAGTATAATACGACAAGGT | |
| | GAGGAACTAAACCATGGCCAAGTTGACCAGTGCCGTTCCGGTGCTCACCGCGCGCGACG | |
| | TCGCCGGAGCGGTCGAGTTCTGGACCGACCGGCTCGGGTTCTCCCGGGACTTCGTGGAG | |
| | GACGACTTCGCCGGTGTGGTCCGGGACGACGTGACCCTGTTCATCAGCGCGGTCCAGGA | |
| | CCAGGTGGTGCCGGACAACACCCTGGCCTGGGTGTGGGTGCGCGGCCTGGACGAGCTGT | |
| | ACGCCGAGTGGTCGGAGGTCGTGTCCACGAACTTCCGGGACGCCTCCGGGCCGGCCATG | |
| | ACCGAGATCGGCGAGCAGCCGTGGGGGCGGGAGTTCGCCCTGCGCGACCCGGCCGGCAA | |
| | CTGCGTGCACTTCGTGGCCGAGGAGCAGGACTGACACGTGCTACGAGATTTCGATTCCA | |
| | CCGCCGCCTTCTATGAAAGGTTGGGCTTCGGAATCGTTTTCCGGGACGCCGGCTGGATG | |
| | ATCCTCCAGCGCGGGGATCTCATGCTGGAGTTCTTCGCCCACCCCAACTTGTTTATTGC | |
| | AGCTTATAATGGTTACAAATAAAGCAATAGCATCACAAATTTCACAAATAAAGCATTTT | |
| | TTTCACTGCATTCTAGTTGTGGTTTGTCCAAACTCATCAATGTATCTTATCATGTCTGT | |
| | ATACCGTCGACCTCTAGCTAGAGCTTGGCGTAATCATGGTCATAGCTGTTTCCTGTGTG | |
| | AAATTGTTATCCGCTCACAATTCCACACAACATACGAGCCGGAAGCATAAAGTGTAAAG | |
| | CCTGGGGTGCCTAATGAGTGAGCTAACTCACATTAATTGCGTTGCGCTCACTGCCCGCT | |
| | TTCCAGTCGGGAAACCTGTCGTGCCAGCTGCATTAATGAATCGGCCAACGCGCGGGGAG | |
| | AGGCGGTTTGCGTATTGGGCGCTCTTCCGCTTCCTCGCTCACTGACTCGCTGCGCTCGG | |
| | TCGTTCGGCTGCGGCGAGCGGTATCAGCTCACTCAAAGGCGGTAATACGGTTATCCACA | |
| | GAATCAGGGGATAACGCAGGAAAGAACATGTGAGCAAAAGGCCAGCAAAAGGCCAGGAA | |
| | CCGTAAAAAGGCCGCGTTGCTGGCGTTTTTCCATAGGCTCCGCCCCCCTGACGAGCATC | |
| | ACAAAAATCGACGCTCAAGTCAGAGGTGGCGAAACCCGACAGGACTATAAAGATACCAG | |
| | GCGTTTCCCCCTGGAAGCTCCCTCGTGCGCTCTCCTGTTCCGACCCTGCCGCTTACCGG | |
| | ATACCTGTCCGCCTTTCTCCCTTCGGGAAGCGTGGCGCTTTCTCATAGCTCACGCTGTA | |
| | GGTATCTCAGTTCGGTGTAGGTCGTTCGCTCCAAGCTGGGCTGTGTGCACGAACCCCCC | |
| | GTTCAGCCCGACCGCTGCGCCTTATCCGGTAACTATCGTCTTGAGTCCAACCCGGTAAG | |
| | ACACGACTTATCGCCACTGGCAGCAGCCACTGGTAACAGGATTAGCAGAGCGAGGTATG | |
| | TAGGCGGTGCTACAGAGTTCTTGAAGTGGTGGCCTAACTACGGCTACACTAGAAGAACA | |
| | GTATTTGGTATCTGCGCTCTGCTGAAGCCAGTTACCTTCGGAAAAAGAGTTGGTAGCTC | |
| | TTGATCCGGCAAACAAACCACCGCTGGTAGCGGTGGTTTTTTTGTTTGCAAGCAGCAGA | |
| | TTACGCGCAGAAAAAAAGGATCTCAAGAAGATCCTTTGATCTTTTCTACGGGGTCTGAC | |
| | GCTCAGTGGAACGAAAACTCACGTTAAGGGATTTTGGTCATGAGATTATCAAAAAGGAT | |
| | CTTCACCTAGATCCTTTTAAATTAAAAATGAAGTTTTAAATCAATCTAAAGTATATATG | |
| | AGTAAACTTGGTCTGACAGTTACCAATGCTTAATCAGTGAGGCACCTATCTCAGCGATC | |
| | TGTCTATTTCGTTCATCCATAGTTGCCTGACTCCCCGTCGTGTAGATAACTACGATACG | |
| | GGAGGGCTTACCATCTGGCCCCAGTGCTGCAATGATACCGCGAGACCCACGCTCACCGG | |
| | CTCCAGATTTATCAGCAATAAACCAGCCAGCCGGAAGGGCCGAGCGCAGAAGTGGTCCT | |
| | GCAACTTTATCCGCCTCCATCCAGTCTATTAATTGTTGCCGGGAAGCTAGAGTAAGTAG | |
| | TTCGCCAGTTAATAGTTTGCGCAACGTTGTTGCCATTGCTACAGGCATCGTGGTGTCAC | |
| | GCTCGTCGTTTGGTATGGCTTCATTCAGCTCCGGTTCCCAACGATCAAGGCGAGTTACA | |
| | TGATCCCCCATGTTGTGCAAAAAAGCGGTTAGCTCCTTCGGTCCTCCGATCGTTGTCAG | |
| | AAGTAAGTTGGCCGCAGTGTTATCACTCATGGTTATGGCAGCACTGCATAATTCTCTTA | |
| | CTGTCATGCCATCCGTAAGATGCTTTTCTGTGACTGGTGAGTACTCAACCAAGTCATTC | |
| | TGAGAATAGTGTATGCGGCGACCGAGTTGCTCTTGCCCGGCGTCAATACGGGATAATAC | |
| | CGCGCCACATAGCAGAACTTTAAAAGTGCTCATCATTGGAAAACGTTCTTCGGGGCGAA | |
| | AACTCTCAAGGATCTTACCGCTGTTGAGATCCAGTTCGATGTAACCCACTCGTGCACCC | |
| | AACTGATCTTCAGCATCTTTTACTTTCACCAGCGTTTCTGGGTGAGCAAAAACAGGAAG | |
| | GCAAAATGCCGCAAAAAAGGGAATAAGGGCGACACGGAAATGTTGAATACTCATACTCT | |
| | TCCTTTTTCAATATTATTGAAGCATTTATCAGGGTTATTGTCTCATGAGCGGATACATA | |
| | TTTGAATGTATTTAGAAAAATAAACAAATAGGGGTTCCGCGCACATTTCCCCGAAAAGT | |
| | GCCACCTGACGTCGACGGATCGGGAGATCTCCCGATCCCCTATGGTGCACTCTCAGTAC | |
| | AATCTGCTCTGATGCCGCATAGTTAAGCCAGTATCTGCTCCCTGCTTGTGTGTTGGAGG | |
| | TCGCTGAGTAGTGCGCGAGCAAAATTTAAGCTACAACAAGGCAAGGCTTGACCGACAAT | |
| | TGCATGAAGAATCTGCTTAGGGTTAGGCGTTTTGCGCTGCTTCGCGATGTACGGGCCAG | |
| | ATATACGCGTT | |
| 75 | gacgcattggacgattttgatctggatatgctgggaagtgacgccctcgatgattttga | VP64- |
| | ccttgacatgcttggttcggatgcccttgatgactttgacctcgacatgctcggcagtg | dSpCas9-NLS- |
| | acgcccttgatgatttcgacctggacatgGTTAACCCAAAGAAGAAGCGGAAGGTCGGT | VP64 (nt) |
| | ATCCACGGAGTCCCAGCAGCCGACAAGAAGTACTCCATTGGGCTCGCCATCGGCACAAA | |
| | CAGCGTCGGCTGGGCCGTCATTACGGACGAGTACAAGGTGCCGAGCAAAAAATTCAAAG | |
| | TTCTGGGCAATACCGATCGCCACAGCATAAAGAAGAACCTCATTGGCGCCCTCCTGTTC | |
| | GACTCCGGGGAAACCGCCGAAGCCACGCGGCTCAAAAGAACAGCACGGCGCAGATATAC | |
| | CCGCAGAAAGAATCGGATCTGCTACCtgcaGGAGATCTTTAGTAATGAGATGGCTAAGG | |
| | TGGATGACTCTTTCTTCCATAGGCTGGAGGAGTCCTTTTTGGTGGAGGAGGATAAAAAG | |
| | CACGAGCGCCACCCAATCTTTGGCAATATCGTGGACGAGGTGGCGTACCATGAAAAGTA | |
| | CCCAACCATATATCATCTGAGGAAGAAGCTTGTAGACAGTACTGATAAGGCTGACTTGC | |
| | GGTTGATCTATCTCGCGCTGGCGCATATGATCAAATTTCGGGGACACTTCCTCATCGAG | |
| | GGGGACCTGAACCCAGACAACAGCGATGTCGACAAACTCTTTATCCAACTGGTTCAGAC | |
| | TTACAATCAGCTTTTCGAAGAGAACCCGATCAACGCATCCGGAGTTGACGCCAAAGCAA | |
| | TCCTGAGCGCTAGGCTGTCCAAATCCCGGCGGCTCGAAAACCTCATCGCACAGCTCCCT | |
| | GGGGAGAAGAAGAACGGCCTGTTTGGTAATCTTATCGCCCTGTCACTCGGGCTGACCCC | |
| | CAACTTTAAATCTAACTTCGACCTGGCCGAAGATGCCAAGCTTCAACTGAGCAAAGACA | |
| | CCTACGATGATGATCTCGACAATCTGCTGGCCCAGATCGGCGACCAGTACGCAGACCTT | |
| | TTTTTGGCGGCAAAGAACCTGTCAGACGCCATTCTGCTGAGTGATATTCTGCGAGTGAA | |
| | CACGGAGATCACCAAAGCTCCGCTGAGCGCTAGTATGATCAAGCGCTATGATGAGCACC | |
| | ACCAAGACTTGACTTTGCTGAAGGCCCTTGTCAGACAGCAACTGCCTGAGAAGTACAAG | |
| | GAAATTTTCTTCGATCAGTCTAAAAATGGCTACGCCGGATACATTGACGGCGGAGCAAG | |
| | CCAGGAGGAATTTTACAAATTTATTAAGCCCATCTTGGAAAAAATGGACGGCACCGAGG | |
| | AGCTGCTGGTAAAGCTTAACAGAGAAGATCTGTTGCGCAAACAGCGCACTTTCGACAAT | |
| | GGAAGCATCCCCCACCAGATTCACCTGGGCGAACTGCACGCTATCCTCAGGCGGCAAGA | |
| | GGATTTCTACCCCTTTTTGAAAGATAACAGGGAAAAGATTGAGAAAATCCTCACATTTC | |
| | GGATACCCTACTATGTAGGCCCCCTCGCCCGGGGAAATTCCAGATTCGCGTGGATGACT | |
| | CGCAAATCAGAAGAGACCATCACTCCCTGGAACTTCGAGGAAGTCGTGGATAAGGGGGC | |
| | CTCTGCCCAGTCCTTCATCGAAAGGATGACTAACTTTGATAAAAATCTGCCTAACGAAA | |
| | AGGTGCTTCCTAAACACTCTCTGCTGTACGAGTACTTCACAGTTTATAACGAGCTCACC | |
| | AAGGTCAAATACGTCACAGAAGGGATGAGAAAGCCAGCATTCCTGTCTGGAGAGCAGAA | |
| | GAAAGCTATCGTGGACCTCCTCTTCAAGACGAACCGGAAAGTTACCGTGAAACAGCTCA | |
| | AAGAAGACTATTTCAAAAAGATTGAATGTTTCGACTCTGTTGAAATCAGCGGAGTGGAG | |
| | GATCGCTTCAACGCATCCCTGGGAACGTATCACGATCTCCTGAAAATCATTAAAGACAA | |
| | GGACTTCCTGGACAATGAGGAGAACGAGGACATTCTTGAGGACATTGTCCTCACCCTTA | |
| | CGTTGTTTGAAGATAGGGAGATGATTGAAGAACGCTTGAAAACTTACGCTCATCTCTTC | |
| | GACGACAAAGTCATGAAACAGCTCAAGAGGCGCCGATATACAGGATGGGGGCGGCTGTC | |
| | AAGAAAACTGATCAATGGgatcCGAGACAAGCAGAGTGGAAAGACAATCCTGGATTTTC | |
| | TTAAGTCCGATGGATTTGCCAACCGGAACTTCATGCAGTTGATCCATGATGACTCTCTC | |
| | ACCTTTAAGGAGGACATCCAGAAAGCACAAGTTTCTGGCCAGGGGGACAGTCTTCACGA | |
| | GCACATCGCTAATCTTGCAGGTAGCCCAGCTATCAAAAAGGGAATACTGCAGACCGTTA | |
| | AGGTCGTGGATGAACTCGTCAAAGTAATGGGAAGGCATAAGCCCGAGAATATCGTTATC | |
| | GAGATGGCCCGAGAGAACCAAACTACCCAGAAGGGACAGAAGAACAGTAGGGAAAGGAT | |
| | GAAGAGGATTGAAGAGGGTATAAAAGAACTGGGGTCCCAAATCCTTAAGGAACACCCAG | |
| | TTGAAAACACCCAGCTTCAGAATGAGAAGCTCTACCTGTACTACCTGCAGAACGGCAGG | |
| | GACATGTACGTGGATCAGGAACTGGACATCAATCGGCTCTCCGACTACGACGTGGATGC | |
| | CATCGTGCCCCAGTCTTTTCTCAAAGATGATTCTATTGATAATAAAGTGTTGACAAGAT | |
| | CCGATAAAAATAGAGGGAAGAGTGATAACGTCCCCTCAGAAGAAGTTGTCAAGAAAATG | |
| | AAAAATTATTGGCGGCAGCTGCTGAACGCCAAACTGATCACACAACGGAAGTTCGATAA | |
| | TCTGACTAAGGCTGAACGAGGTGGCCTGTCTGAGTTGGATAAAGCCGGCTTCATCAAAA | |
| | GGCAGCTTGTTGAGACACGCCAGATCACCAAgcacGTGGCCCAAATTCTCGATTCACGC | |
| | ATGAACACCAAGTACGATGAAAATGACAAACTGATTCGAGAGGTGAAAGTTATTACTCT | |
| | GAAGTCTAAGCTGGTCTCAGATTTCAGAAAGGACTTTCAGTTTTATAAGGTGAGAGAGA | |
| | TCAACAATTACCACCATGCGCATGATGCCTACCTGAATGCAGTGGTAGGCACTGCACTT | |
| | ATCAAAAAATATCCCAAGCTTGAATCTGAATTTGTTTACGGAGACTATAAAGTGTACGA | |
| | TGTTAGGAAAATGATCGCAAAGTCTGAGCAGGAAATAGGCAAGGCCACCGCTAAGTACT | |
| | TCTTTTACAGCAATATTATGAATTTTTTCAAGACCGAGATTACACTGGCCAATGGAGAG | |
| | ATTCGGAAGCGACCACTTATCGAAACAAACGGAGAAACAGGAGAAATCGTGTGGGACAA | |
| | GGGTAGGGATTTCGCGACAGTCCGGAAGGTCCTGTCCATGCCGCAGGTGAACATCGTTA | |
| | AAAAGACCGAAGTACAGACCGGAGGCTTCTCCAAGGAAAGTATCCTCCCGAAAAGGAAC | |
| | AGCGACAAGCTGATCGCACGCAAAAAAGATTGGGACCCCAAGAAATACGGCGGATTCGA | |
| | TTCTCCTACAGTCGCTTACAGTGTACTGGTTGTGGCCAAAGTGGAGAAAGGGAAGTCTA | |
| | AAAAACTCAAAAGCGTCAAGGAACTGCTGGGCATCACAATCATGGAGCGATCAAGCTTC | |
| | GAAAAAAACCCCATCGACTTTCTCGAGGCGAAAGGATATAAAGAGGTCAAAAAAGACCT | |
| | CATCATTAAGCTTCCCAAGTACTCTCTCTTTGAGCTTGAAAACGGCCGGAAACGAATGC | |
| | TCGCTAGTGCGGGCGAGCTGCAGAAAGGTAACGAGCTGGCACTGCCCTCTAAATACGTT | |
| | AATTTCTTGTATCTGGCCAGCCACTATGAAAAGCTCAAAGGGTCTCCCGAAGATAATGA | |
| | GCAGAAGCAGCTGTTCGTGGAACAACACAAACACTACCTTGATGAGATCATCGAGCAAA | |
| | TAAGCGAATTCTCCAAAAGAGTGATCCTCGCCGACGCTAACCTCGATAAGGTGCTTTCT | |
| | GCTTACAATAAGCACAGGGATAAGCCCATCAGGGAGCAGGCAGAAAACATTATCCACTT | |
| | GTTTACTCTGACCAACTTGGGCGCGCCTGCAGCCTTCAAGTACTTCGACACCACCATAG | |
| | ACAGAAAGCGGTACACCTCTACAAAGGAGGTCCTGGACGCCACACTGATTCATCAGTCA | |
| | ATTACGGGGCTCTATGAAACAAGAATCGACCTCTCTCAGCTCGGTGGAGACAAAAGGCC | |
| | GGCGGCCACGAAAAAGGCCGGCCAGGCAAAAAAGAAAAAGGctagCcgcgccgacgcgc | |
| | tggacgatttcgatctcgacatgctgggttctgatgccctcgatgactttgacctggat | |
| | atgttgggaagcgacgcattggatgactttgatctggacatgctcggctccgatgctct | |
| | ggacgatttcgatctcgatatgtta | |
| 76 | GACAAGAAGTACTCCATTGGGCTCGCCATCGGCACAAACAGCGTCGGCTGGGCCGTCAT | dSpCas9 (nt) |
| | TACGGACGAGTACAAGGTGCCGAGCAAAAAATTCAAAGTTCTGGGCAATACCGATCGCC | |
| | ACAGCATAAAGAAGAACCTCATTGGCGCCCTCCTGTTCGACTCCGGGGAAACCGCCGAA | |
| | GCCACGCGGCTCAAAAGAACAGCACGGCGCAGATATACCCGCAGAAAGAATCGGATCTG | |
| | CTACCtgcaGGAGATCTTTAGTAATGAGATGGCTAAGGTGGATGACTCTTTCTTCCATA | |
| | GGCTGGAGGAGTCCTTTTTGGTGGAGGAGGATAAAAAGCACGAGCGCCACCCAATCTTT | |
| | GGCAATATCGTGGACGAGGTGGCGTACCATGAAAAGTACCCAACCATATATCATCTGAG | |
| | GAAGAAGCTTGTAGACAGTACTGATAAGGCTGACTTGCGGTTGATCTATCTCGCGCTGG | |
| | CGCATATGATCAAATTTCGGGGACACTTCCTCATCGAGGGGGACCTGAACCCAGACAAC | |
| | AGCGATGTCGACAAACTCTTTATCCAACTGGTTCAGACTTACAATCAGCTTTTCGAAGA | |
| | GAACCCGATCAACGCATCCGGAGTTGACGCCAAAGCAATCCTGAGCGCTAGGCTGTCCA | |
| | AATCCCGGCGGCTCGAAAACCTCATCGCACAGCTCCCTGGGGAGAAGAAGAACGGCCTG | |
| | TTTGGTAATCTTATCGCCCTGTCACTCGGGCTGACCCCCAACTTTAAATCTAACTTCGA | |
| | CCTGGCCGAAGATGCCAAGCTTCAACTGAGCAAAGACACCTACGATGATGATCTCGACA | |
| | ATCTGCTGGCCCAGATCGGCGACCAGTACGCAGACCTTTTTTTGGCGGCAAAGAACCTG | |
| | TCAGACGCCATTCTGCTGAGTGATATTCTGCGAGTGAACACGGAGATCACCAAAGCTCC | |
| | GCTGAGCGCTAGTATGATCAAGCGCTATGATGAGCACCACCAAGACTTGACTTTGCTGA | |
| | AGGCCCTTGTCAGACAGCAACTGCCTGAGAAGTACAAGGAAATTTTCTTCGATCAGTCT | |
| | AAAAATGGCTACGCCGGATACATTGACGGCGGAGCAAGCCAGGAGGAATTTTACAAATT | |
| | TATTAAGCCCATCTTGGAAAAAATGGACGGCACCGAGGAGCTGCTGGTAAAGCTTAACA | |
| | GAGAAGATCTGTTGCGCAAACAGCGCACTTTCGACAATGGAAGCATCCCCCACCAGATT | |
| | CACCTGGGCGAACTGCACGCTATCCTCAGGCGGCAAGAGGATTTCTACCCCTTTTTGAA | |
| | AGATAACAGGGAAAAGATTGAGAAAATCCTCACATTTCGGATACCCTACTATGTAGGCC | |
| | CCCTCGCCCGGGGAAATTCCAGATTCGCGTGGATGACTCGCAAATCAGAAGAGACCATC | |
| | ACTCCCTGGAACTTCGAGGAAGTCGTGGATAAGGGGGCCTCTGCCCAGTCCTTCATCGA | |
| | AAGGATGACTAACTTTGATAAAAATCTGCCTAACGAAAAGGTGCTTCCTAAACACTCTC | |
| | TGCTGTACGAGTACTTCACAGTTTATAACGAGCTCACCAAGGTCAAATACGTCACAGAA | |
| | GGGATGAGAAAGCCAGCATTCCTGTCTGGAGAGCAGAAGAAAGCTATCGTGGACCTCCT | |
| | CTTCAAGACGAACCGGAAAGTTACCGTGAAACAGCTCAAAGAAGACTATTTCAAAAAGA | |
| | TTGAATGTTTCGACTCTGTTGAAATCAGCGGAGTGGAGGATCGCTTCAACGCATCCCTG | |
| | GGAACGTATCACGATCTCCTGAAAATCATTAAAGACAAGGACTTCCTGGACAATGAGGA | |
| | GAACGAGGACATTCTTGAGGACATTGTCCTCACCCTTACGTTGTTTGAAGATAGGGAGA | |
| | TGATTGAAGAACGCTTGAAAACTTACGCTCATCTCTTCGACGACAAAGTCATGAAACAG | |
| | CTCAAGAGGCGCCGATATACAGGATGGGGGCGGCTGTCAAGAAAACTGATCAATGGgat | |
| | CCGAGACAAGCAGAGTGGAAAGACAATCCTGGATTTTCTTAAGTCCGATGGATTTGCCA | |
| | ACCGGAACTTCATGCAGTTGATCCATGATGACTCTCTCACCTTTAAGGAGGACATCCAG | |
| | AAAGCACAAGTTTCTGGCCAGGGGGACAGTCTTCACGAGCACATCGCTAATCTTGCAGG | |
| | TAGCCCAGCTATCAAAAAGGGAATACTGCAGACCGTTAAGGTCGTGGATGAACTCGTCA | |
| | AAGTAATGGGAAGGCATAAGCCCGAGAATATCGTTATCGAGATGGCCCGAGAGAACCAA | |
| | ACTACCCAGAAGGGACAGAAGAACAGTAGGGAAAGGATGAAGAGGATTGAAGAGGGTAT | |
| | AAAAGAACTGGGGTCCCAAATCCTTAAGGAACACCCAGTTGAAAACACCCAGCTTCAGA | |
| | ATGAGAAGCTCTACCTGTACTACCTGCAGAACGGCAGGGACATGTACGTGGATCAGGAA | |
| | CTGGACATCAATCGGCTCTCCGACTACGACGTGGATGCCATCGTGCCCCAGTCTTTTCT | |
| | CAAAGATGATTCTATTGATAATAAAGTGTTGACAAGATCCGATAAAAATAGAGGGAAGA | |
| | GTGATAACGTCCCCTCAGAAGAAGTTGTCAAGAAAATGAAAAATTATTGGCGGCAGCTG | |
| | CTGAACGCCAAACTGATCACACAACGGAAGTTCGATAATCTGACTAAGGCTGAACGAGG | |
| | TGGCCTGTCTGAGTTGGATAAAGCCGGCTTCATCAAAAGGCAGCTTGTTGAGACACGCC | |
| | AGATCACCAAgcacGTGGCCCAAATTCTCGATTCACGCATGAACACCAAGTACGATGAA | |
| | AATGACAAACTGATTCGAGAGGTGAAAGTTATTACTCTGAAGTCTAAGCTGGTCTCAGA | |
| | TTTCAGAAAGGACTTTCAGTTTTATAAGGTGAGAGAGATCAACAATTACCACCATGCGC | |
| | ATGATGCCTACCTGAATGCAGTGGTAGGCACTGCACTTATCAAAAAATATCCCAAGCTT | |
| | GAATCTGAATTTGTTTACGGAGACTATAAAGTGTACGATGTTAGGAAAATGATCGCAAA | |
| | GTCTGAGCAGGAAATAGGCAAGGCCACCGCTAAGTACTTCTTTTACAGCAATATTATGA | |
| | ATTTTTTCAAGACCGAGATTACACTGGCCAATGGAGAGATTCGGAAGCGACCACTTATC | |
| | GAAACAAACGGAGAAACAGGAGAAATCGTGTGGGACAAGGGTAGGGATTTCGCGACAGT | |
| | CCGGAAGGTCCTGTCCATGCCGCAGGTGAACATCGTTAAAAAGACCGAAGTACAGACCG | |
| | GAGGCTTCTCCAAGGAAAGTATCCTCCCGAAAAGGAACAGCGACAAGCTGATCGCACGC | |
| | AAAAAAGATTGGGACCCCAAGAAATACGGCGGATTCGATTCTCCTACAGTCGCTTACAG | |
| | TGTACTGGTTGTGGCCAAAGTGGAGAAAGGGAAGTCTAAAAAACTCAAAAGCGTCAAGG | |
| | AACTGCTGGGCATCACAATCATGGAGCGATCAAGCTTCGAAAAAAACCCCATCGACTTT | |
| | CTCGAGGCGAAAGGATATAAAGAGGTCAAAAAAGACCTCATCATTAAGCTTCCCAAGTA | |
| | CTCTCTCTTTGAGCTTGAAAACGGCCGGAAACGAATGCTCGCTAGTGCGGGCGAGCTGC | |
| | AGAAAGGTAACGAGCTGGCACTGCCCTCTAAATACGTTAATTTCTTGTATCTGGCCAGC | |
| | CACTATGAAAAGCTCAAAGGGTCTCCCGAAGATAATGAGCAGAAGCAGCTGTTCGTGGA | |
| | ACAACACAAACACTACCTTGATGAGATCATCGAGCAAATAAGCGAATTCTCCAAAAGAG | |
| | TGATCCTCGCCGACGCTAACCTCGATAAGGTGCTTTCTGCTTACAATAAGCACAGGGAT | |
| | AAGCCCATCAGGGAGCAGGCAGAAAACATTATCCACTTGTTTACTCTGACCAACTTGGG | |
| | CGCGCCTGCAGCCTTCAAGTACTTCGACACCACCATAGACAGAAAGCGGTACACCTCTA | |
| | CAAAGGAGGTCCTGGACGCCACACTGATTCATCAGTCAATTACGGGGCTCTATGAAACA | |
| | AGAATCGACCTCTCTCAGCTCGGTGGAGAC | |
| 77 | DALDDFDLDMLGSDALDDFDLDMLGSDALDDFDLDMLGSDALDDFDLDMVNPKKKRKVG | VP64- |
| | IHGVPAADKKYSIGLAIGTNSVGWAVITDEYKVPSKKFKVLGNTDRHSIKKNLIGALLF | dSpCas9-NLS- |
| | DSGETAEATRLKRTARRRYTRRKNRICYLQEIFSNEMAKVDDSFFHRLEESFLVEEDKK | VP64 (aa) |
| | HERHPIFGNIVDEVAYHEKYPTIYHLRKKLVDSTDKADLRLIYLALAHMIKERGHFLIE | |
| | GDLNPDNSDVDKLFIQLVQTYNQLFEENPINASGVDAKAILSARLSKSRRLENLIAQLP | |
| | GEKKNGLFGNLIALSLGLTPNFKSNFDLAEDAKLQLSKDTYDDDLDNLLAQIGDQYADL | |
| | FLAAKNLSDAILLSDILRVNTEITKAPLSASMIKRYDEHHQDLTLLKALVRQQLPEKYK | |
| | EIFFDQSKNGYAGYIDGGASQEEFYKFIKPILEKMDGTEELLVKLNREDLLRKQRTFDN | |
| | GSIPHQIHLGELHAILRRQEDFYPFLKDNREKIEKILTFRIPYYVGPLARGNSRFAWMT | |
| | RKSEETITPWNFEEVVDKGASAQSFIERMTNFDKNLPNEKVLPKHSLLYEYFTVYNELT | |
| | KVKYVTEGMRKPAFLSGEQKKAIVDLLFKTNRKVTVKQLKEDYFKKIECFDSVEISGVE | |
| | DRFNASLGTYHDLLKIIKDKDELDNEENEDILEDIVLTLTLFEDREMIEERLKTYAHLF | |
| | DDKVMKQLKRRRYTGWGRLSRKLINGIRDKQSGKTILDFLKSDGFANRNFMQLIHDDSL | |
| | TFKEDIQKAQVSGQGDSLHEHIANLAGSPAIKKGILQTVKVVDELVKVMGRHKPENIVI | |
| | EMARENQTTQKGQKNSRERMKRIEEGIKELGSQILKEHPVENTQLQNEKLYLYYLQNGR | |
| | DMYVDQELDINRLSDYDVDAIVPQSFLKDDSIDNKVLTRSDKNRGKSDNVPSEEVVKKM | |
| | KNYWRQLLNAKLITQRKFDNLTKAERGGLSELDKAGFIKRQLVETRQITKHVAQILDSR | |
| | MNTKYDENDKLIREVKVITLKSKLVSDFRKDFQFYKVREINNYHHAHDAYLNAVVGTAL | |
| | IKKYPKLESEFVYGDYKVYDVRKMIAKSEQEIGKATAKYFFYSNIMNFFKTEITLANGE | |
| | IRKRPLIETNGETGEIVWDKGRDFATVRKVLSMPQVNIVKKTEVQTGGFSKESILPKRN | |
| | SDKLIARKKDWDPKKYGGFDSPTVAYSVLVVAKVEKGKSKKLKSVKELLGITIMERSSF | |
| | EKNPIDFLEAKGYKEVKKDLIIKLPKYSLFELENGRKRMLASAGELQKGNELALPSKYV | |
| | NFLYLASHYEKLKGSPEDNEQKQLFVEQHKHYLDEIIEQISEFSKRVILADANLDKVLS | |
| | AYNKHRDKPIREQAENIIHLFTLTNLGAPAAFKYFDTTIDRKRYTSTKEVLDATLIHQS | |
| | ITGLYETRIDLSQLGGDKRPAATKKAGQAKKKKASRADALDDFDLDMLGSDALDDEDLD | |
| | MLGSDALDDFDLDMLGSDALDDEDLDML | |
| 78 | DKKYSIGLAIGTNSVGWAVITDEYKVPSKKFKVLGNTDRHSIKKNLIGALLFDSGETAE | dSpCas9 (aa) |
| | ATRLKRTARRRYTRRKNRICYLQEIFSNEMAKVDDSFFHRLEESFLVEEDKKHERHPIF | WT Sp Cas9 |
| | GNIVDEVAYHEKYPTIYHLRKKLVDSTDKADLRLIYLALAHMIKFRGHFLIEGDLNPDN | (aa) |
| | SDVDKLFIQLVQTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGDQYADLFLAAKNL | |
| | SDAILLSDILRVNTEITKAPLSASMIKRYDEHHQDLTLLKALVRQQLPEKYKEIFFDQS | |
| | KNGYAGYIDGGASQEEFYKFIKPILEKMDGTEELLVKLNREDLLRKQRTFDNGSIPHQI | |
| | HLGELHAILRRQEDFYPFLKDNREKIEKILTFRIPYYVGPLARGNSRFAWMTRKSEETI | |
| | TPWNFEEVVDKGASAQSFIERMTNFDKNLPNEKVLPKHSLLYEYFTVYNELTKVKYVTE | |
| | GMRKPAFLSGEQKKAIVDLLFKTNRKVTVKQLKEDYFKKIECFDSVEISGVEDRFNASL | |
| | GTYHDLLKIIKDKDFLDNEENEDILEDIVLTLTLFEDREMIEERLKTYAHLFDDKVMKQ | |
| | LKRRRYTGWGRLSRKLINGIRDKQSGKTILDFLKSDGFANRNFMQLIHDDSLTFKEDIQ | |
| | KAQVSGQGDSLHEHIANLAGSPAIKKGILQTVKVVDELVKVMGRHKPENIVIEMARENQ | |
| | TTQKGQKNSRERMKRIEEGIKELGSQILKEHPVENTQLQNEKLYLYYLQNGRDMYVDQE | |
| | LDINRLSDYDVDAIVPQSFLKDDSIDNKVLTRSDKNRGKSDNVPSEEVVKKMKNYWRQL | |
| | LNAKLITQRKFDNLTKAERGGLSELDKAGFIKRQLVETRQITKHVAQILDSRMNTKYDE | |
| | NDKLIREVKVITLKSKLVSDFRKDFQFYKVREINNYHHAHDAYLNAVVGTALIKKYPKL | |
| | ESEFVYGDYKVYDVRKMIAKSEQEIGKATAKYFFYSNIMNFFKTEITLANGEIRKRPLI | |
| | ETNGETGEIVWDKGRDFATVRKVLSMPQVNIVKKTEVQTGGFSKESILPKRNSDKLIAR | |
| | KKDWDPKKYGGFDSPTVAYSVLVVAKVEKGKSKKLKSVKELLGITIMERSSFEKNPIDF | |
| | LEAKGYKEVKKDLIIKLPKYSLFELENGRKRMLASAGELQKGNELALPSKYVNFLYLAS | |
| | HYEKLKGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGLYET | |
| | RIDLSQLGGD | |
| 79 | MDKKYSIGLDIGTNSVGWAVITDEYKVPSKKFKVLGNTDRHSIKKNLIGALLFDSGETA | |
| | EATRLKRTARRRYTRRKNRICYLQEIFSNEMAKVDDSFFHRLEESFLVEEDKKHERHPI | |
| | FGNIVDEVAYHEKYPTIYHLRKKLVDSTDKADLRLIYLALAHMIKFRGHFLIEGDLNPD | |
| | NSDVDKLFIQLVQTYNQLFEENPINASGVDAKAILSARLSKSRRLENLIAQLPGEKKNG | |
| | LFGNLIALSLGLTPNFKSNFDLAEDAKLQLSKDTYDDDLDNLLAQIGDQYADLFLAAKN | |
| | LSDAILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTEDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRENAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLETLTNLGAPAAFKYFDTTIDRKRYTSTKEVLDATLIHQSITGLYE | |
| | TRIDLSQLGGD | |
| 80 | gacgcattggacgattttgatctggatatgctgggaagtgacgccctcgatgattttga | VP64 1 (nt) |
| | ccttgacatgcttggttcggatgcccttgatgactttgacctcgacatgctcggcagtg | |
| | acgcccttgatgatttcgacctggacatg | |
| 81 | DALDDFDLDMLGSDALDDFDLDMLGSDALDDFDLDMLGSDALDDFDLDM | VP64 1 (aa) |
| 82 | gacgcgctggacgatttcgatctcgacatgctgggttctgatgccctcgatgactttga | VP64 2 (nt) |
| | cctggatatgttgggaagcgacgcattggatgactttgatctggacatgctcggctccg | |
| | atgctctggacgatttcgatctcgatatgtta | |
| 83 | DALDDFDLDMLGSDALDDFDLDMLGSDALDDFDLDMLGSDALDDEDLDML | VP64 2 (aa) |
| 84 | AAAAGGCCGGCGGCCACGAAAAAGGCCGGCCAGGCAAAAAAGAAAAAG | NLS (nt) |
| 85 | KRPAATKKAGQAKKKK | NLS (aa) |
| 86 | GGAATTGTGACCCTGCCTCATCGCTACGTGTACCTGATCAGGTTTCTTGCGGACCACGT | Control peptide |
| | CGATGCTAGCCAATCCCTAACTACGAGAACTCGTATTGACACATTCATGTGCGTCGCCT | (nt) |
| | GCTCTTTCGACGGCAGTAATTATACCGCAGTGTTAAGTCCCCAGATACTCGCCATCCGA | |
| | CGAAACTCTTGTGTACGAGATATGCGATTCGTTGCTAGACGTGAGTACACCAAGGCCGT | |
| | GTTC | |
| 87 | AAGAGCAAGAAGGGCAGACAGGAGGCCCTGGAGAGACTGAAGAAGGCCAAGGCCGGCGA | DPOLA (nt) |
| | GAAGTACAAGTACGAGGTGGAGGACTTCACCGGCGTGTACGAGGAGGTGGACGAGGAGC | |
| | AGTACAGCAAGCTGGTGCAGGCCAGACAGGACGACGACTGGATCGTGGACGACGACGGC | |
| | ATCGGCTACGTGGAGGACGGCAGAGAGATCTTCGACGACGACCTGGAGGACGACGCCCT | |
| | GGAC | |
| 88 | AGCAAGCCCGAGAAGATCCTGAAGAAGGGCACCTACGACAAGGCCTACACCGACGAGCT | ENL (nt) |
| | GGTGGAGCTGCACAGAAGACTGATGGCCCTGAGAGAGAGAAACGTGCTGCAGCAGATCG | |
| | TGAACCTGATCGAGGAGACCGGCCACTTCAACGTGACCAACACCACCTTCGACTTCGAC | |
| | CTGTTCAGCCTGGACGAGACCACCGTGAGAAAGCTGCAGAGCTGCCTGGAGGCCGTGGC | |
| | CACC | |
| 89 | GACAGCCTGAGCGGCAGCAGCCTGTACAGCACCAGCGCCAACCTGCCCGTGATGGGCCA | FOXO3 (nt) |
| | CGAGAAGTTCCCCAGCGACCTGGACCTGGACATGTTCAACGGCAGCCTGGAGTGCGACA | |
| | TGGAGAGCATCATCAGAAGCGAGCTGATGGACGCCGACGGCCTGGACTTCAACTTCGAC | |
| | AGCCTGATCAGCACCCAGAACGTGGTGGGCCTGAACGTGGGCAACTTCACCGGCGCCAA | |
| | GCAG | |
| 90 | CCCGAGTGGTTCCACGGCGCCATCAGCAGAGAGGACGCCGAGAACCTGCTGGAGAGCCA | HSH2D (nt) |
| | GCCCCTGGGCAGCTTCCTGATCAGAGTGAGCCACAGCCACGTGGGCTACACCCTGAGCT | |
| | ACAAGGCCCAGAGCAGCTGCTGCCACTTCATGGTGAAGCTGCTGGACGACGGCACCTTC | |
| | ATGATCCCCGGCGAGAAGGTGGCCCACACCAGCCTGGACGCCCTGGTGACCTTCCACCA | |
| | GCAG | |
| 91 | CCCTTCGGCAGCAGCCCCGACGACCTGCTGTGCCCCCACCCCGCTGCTGAAAGCCCCAG | NCOA2 (nt) |
| | CGACGAGGGCGCTCTGCTGGACCAGCTGTACCTGGCCCTGAGAAACTTCGACGGCCTGG | |
| | AGGAGATCGACAGAGCCCTGGGCATCCCCGAGCTGGTGAGCCAGAGCCAGGCCGTGGAC | |
| | CCCGAGCAGTTCAGCAGCCAGGACAGCAACATCATGCTGGAGCAGAAGGCCCCCGTGTT | |
| | CCCC | |
| 92 | CTGAGAAACAGCCTGGACGACCTGGTGGGCCCCCCCAGCAACCTGGAGGGCCAGAGCGA | NCOA3 (nt) |
| | CGAGAGAGCCCTGCTGGACCAGCTGCACACCCTGCTGAGCAACACCGACGCCACCGGCC | |
| | TGGAGGAGATCGACAGAGCCCTGGGCATCCCCGAGCTGGTGAACCAGGGCCAGGCCCTG | |
| | GAGCCCAAGCAGGACGCCTTCCAGGGCCAGGAGGCTGCCGTGATGATGGACCAGAAGGC | |
| | CGGC | |
| 93 | TTCAGAAACCAGTACGACAACGACGTGACCGTGTGGAGCCCCCAGGGCAGAATCCACCA | PSA1 (nt) |
| | GATCGAGTACGCCATGGAGGCCGTGAAGCAGGGCAGCGCCACCGTGGGCCTGAAGAGCA | |
| | AGACCCACGCCGTGCTGGTGGCCCTGAAGAGAGCCCAGAGCGAGCTGGCCGCCCACCAG | |
| | AAGAAGATCCTGCACGTGGACAACCACATCGGCATCAGCATCGCCGGCCTGACCGCCGA | |
| | CGCC | |
| 94 | AGACACGGCCACAGCAGCAGCGACCCCGTGTACCCCTGCGGCATCTGCACCAACGAGGT | PYGO1 (nt) |
| | GAACGACGACCAGGACGCCATCCTGTGCGAGGCCAGCTGCCAGAAGTGGTTCCACAGAA | |
| | TCTGCACCGGCATGACCGAGACCGCCTACGGCCTGCTGACCGCCGAGGCCAGCGCTGTG | |
| | TGGGGATGCGACACCTGCATGGCCGACAAGGACGTGCAGCTGATGAGAACCAGAGAGAC | |
| | CTTC | |
| 95 | GGCCCCATGAGACTGTACGTGGGCAGCCTGCACTTCAACATCACCGAGGACATGCTGAG | RBM39 (nt) |
| | AGGCATCTTCGAGCCCTTCGGCAGAATCGAGAGCATCCAGCTGATGATGGACAGCGAGA | |
| | CCGGCAGAAGCAAGGGCTACGGCTTCATCACCTTCAGCGACAGCGAGTGCGCCAAGAAG | |
| | GCCCTGGAGCAGCTGAACGGCTTCGAGCTGGCCGGCAGACCCATGAAGGTGGGCCACGT | |
| | GACC | |
| 96 | ACCCTGATCAGAAAGGCCGACCTGGAGAACCACAACAAGGACGGCGGCTTCTGGACCGT | HERC2 (nt) |
| | GATCGACGGCAAGGTGTACGACATCAAGGACTTCCAGACCCAGAGCCTGACCGGCAACA | |
| | GCATCCTGGCCCAGTTCGCCGGCGAGGACCCCGTGGTGGCCCTGGAAGCTGCTCTGCAG | |
| | TTCGAGGACACCAGAGAGAGCATGCACGCCTTCTGCGTGGGCCAGTACCTGGAGCCCGA | |
| | CCAG | |
| 97 | CCCTACTACATCAACCACGAGACCCAGACCACCTGCTGGGACCACCCCAAGATGACCGA | DMD control |
| | GCTGTACCAGAGCCTGGCCGACCTGAACAACGTGAGATTCAGCGCCTACAGGACCGCCA | (nt) |
| | TGAAGCTGAGAAGACTGCAGAAGGCCCTGTGCCTGGACCTGCTGAGCCTGAGCGCCGCC | |
| | TGCGACGCCTTGGACCAGCACAACCTGAAGCAGAACGACCAGCCCATGGACATCCTGCA | |
| | GATC | |
| 98 | AACAGATACAGCTGCGTGTGCAGCCCCGGCTTCACCGGCCAGAGATGCAACATCGACAT | NOTCH2 (nt) |
| | CGACGAGTGCGCCAGCAACCCCTGCAGAAAGGGCGCCACCTGCATCAACGGCGTGAACG | |
| | GCTTCAGATGCATCTGCCCCGAGGGCCCCCACCACCCCAGCTGCTACAGCCAGGTGAAC | |
| | GAGTGCCTGAGCAACCCCTGCATCCACGGCAACTGCACCGGCGGCCTGAGCGGCTACAA | |
| | GTGC | |
| 99 | GIVTLPHRYVYLIRFLADHVDASQSLTTRTRIDTFMCVACSFDGSNYTAVLSPQILAIR | Control peptide |
| | RNSCVRDMRFVARREYTKAVE | (aa) |
| 100 | KSKKGRQEALERLKKAKAGEKYKYEVEDFTGVYEEVDEEQYSKLVQARQDDDWIVDDDG | DPOLA (aa) |
| | IGYVEDGREIFDDDLEDDALD | |
| 101 | SKPEKILKKGTYDKAYTDELVELHRRLMALRERNVLQQIVNLIEETGHENVINTTEDED | ENL (aa) |
| | LFSLDETTVRKLQSCLEAVAT | |
| 102 | DSLSGSSLYSTSANLPVMGHEKFPSDLDLDMENGSLECDMESIIRSELMDADGLDENED | FOXO3 (aa) |
| | SLISTQNVVGLNVGNFTGAKQ | |
| 103 | PEWFHGAISREDAENLLESQPLGSFLIRVSHSHVGYTLSYKAQSSCCHFMVKLLDDGTF | HSH2D (aa) |
| | MIPGEKVAHTSLDALVTFHQQ | |
| 104 | PFGSSPDDLLCPHPAAESPSDEGALLDQLYLALRNEDGLEEIDRALGIPELVSQSQAVD | NCOA2 (aa) |
| | PEQFSSQDSNIMLEQKAPVFP | |
| 105 | LRNSLDDLVGPPSNLEGQSDERALLDQLHTLLSNTDATGLEEIDRALGIPELVNQGQAL | NCOA3 (aa) |
| | EPKQDAFQGQEAAVMMDQKAG | |
| 106 | FRNQYDNDVTVWSPQGRIHQIEYAMEAVKQGSATVGLKSKTHAVLVALKRAQSELAAHQ | PSA1 (aa) |
| | KKILHVDNHIGISIAGLTADA | |
| 107 | RHGHSSSDPVYPCGICTNEVNDDQDAILCEASCQKWFHRICTGMTETAYGLLTAEASAV | PYGO1 (aa) |
| | WGCDTCMADKDVQLMRTRETF | |
| 108 | GPMRLYVGSLHFNITEDMLRGIFEPFGRIESIQLMMDSETGRSKGYGFITFSDSECAKK | RBM39 (aa) |
| | ALEQLNGFELAGRPMKVGHVT | |
| 109 | TLIRKADLENHNKDGGFWTVIDGKVYDIKDFQTQSLTGNSILAQFAGEDPVVALEAALQ | HERC2 (aa) |
| | FEDTRESMHAFCVGQYLEPDQ | |
| 110 | PYYINHETQTTCWDHPKMTELYQSLADLNNVRESAYRTAMKLRRLQKALCLDLLSLSAA | DMD (aa) |
| | CDALDQHNLKQNDQPMDILQI | |
| 111 | NRYSCVCSPGFTGQRCNIDIDECASNPCRKGATCINGVNGFRCICPEGPHHPSCYSQVN | NOTCH2 (aa) |
| | ECLSNPCIHGNCTGGLSGYKC | |
| 112 | IFKPEELRQALMPTLEALYRQDPESLPFRQPVDPQLLGIPDYFDIVKSPMDLSTIKRKL | p300 core (aa) |
| | DTGQYQEPWQYVDDIWLMFNNAWLYNRKTSRVYKYCSKLSEVFEQEIDPVMQSLGYCCG | |
| | RKLEFSPQTLCCYGKQLCTIPRDATYYSYQNRYHFCEKCFNEIQGESVSLGDDPSQPQT | |
| | TINKEQFSKRKNDTLDPELFVECTECGRKMHQICVLHHEIIWPAGFVCDGCLKKSARTR | |
| | KENKFSAKRLPSTRLGTFLENRVNDFLRRQNHPESGEVTVRVVHASDKTVEVKPGMKAR | |
| | FVDSGEMAESFPYRTKALFAFEEIDGVDLCFFGMHVQEYGSDCPPPNQRRVYISYLDSV | |
| | HFFRPKCLRTAVYHEILIGYLEYVKKLGYTTGHIWACPPSEGDDYIFHCHPPDQKIPKP | |
| | KRLQEWYKKMLDKAVSERIVHDYKDIFKQATEDRLTSAKELPYFEGDFWPNVLEESIKE | |
| | LEQEEEERKREENTSNESTDVTKGDSKNAKKKNNKKTSKNKSSLSRGNKKKPGMPNVSN | |
| | DLSQKLYATMEKHKEVFFVIRLIAGPAANSLPPIVDPDPLIPCDLMDGRDAFLTLARDK | |
| | HLEFSSLRRAQWSTMCMLVELHTQSQD | |
| 113 | MAPVHGDDCEIGASALSDSGSFVSSRARREKKSKKGRQEALERLKKAKAGEKYKYEVED | DNA |
| | FTGVYEEVDEEQYSKLVQARQDDDWIVDDDGIGYVEDGREIFDDDLEDDALDADEKGKD | polymerase |
| | GKARNKDKRNVKKLAVTKPNNIKSMFIACAGKKTADKAVDLSKDGLLGDILQDLNTETP | alpha catalytic |
| | QITPPPVMILKKKRSIGASPNPFSVHTATAVPSGKIASPVSRKEPPLTPVPLKRAEFAG | subunit |
| | DDVQVESTEEEQESGAMEFEDGDFDEPMEVEEVDLEPMAAKAWDKESEPAEEVKQEADS | (DPOLA) |
| | GKGTVSYLGSFLPDVSCWDIDQEGDSSFSVQEVQVDSSHLPLVKGADEEQVFHFYWLDA | isoform 1 |
| | YEDQYNQPGVVFLFGKVWIESAETHVSCCVMVKNIERTLYFLPREMKIDLNTGKETGTP | [Homo sapiens] |
| | ISMKDVYEEFDEKIATKYKIMKFKSKPVEKNYAFEIPDVPEKSEYLEVKYSAEMPQLPQ | (aa) |
| | DLKGETFSHVFGTNTSSLELFLMNRKIKGPCWLEVKSPQLLNQPVSWCKVEAMALKPDL | |
| | VNVIKDVSPPPLVVMAFSMKTMQNAKNHQNEIIAMAALVHHSFALDKAAPKPPFQSHFC | |
| | VVSKPKDCIFPYAFKEVIEKKNVKVEVAATERTLLGFFLAKVHKIDPDIIVGHNIYGFE | |
| | LEVLLQRINVCKAPHWSKIGRLKRSNMPKLGGRSGFGERNATCGRMICDVEISAKELIR | |
| | CKSYHLSELVQQILKTERVVIPMENIQNMYSESSQLLYLLEHTWKDAKFILQIMCELNV | |
| | LPLALQITNIAGNIMSRTLMGGRSERNEFLLLHAFYENNYIVPDKQIFRKPQQKLGDED | |
| | EEIDGDTNKYKKGRKKAAYAGGLVLDPKVGFYDKFILLLDENSLYPSIIQEFNICFTTV | |
| | QRVASEAQKVTEDGEQEQIPELPDPSLEMGILPREIRKLVERRKQVKQLMKQQDLNPDL | |
| | ILQYDIRQKALKLTANSMYGCLGFSYSRFYAKPLAALVTYKGREILMHTKEMVQKMNLE | |
| | VIYGDTDSIMINTNSTNLEEVFKLGNKVKSEVNKLYKLLEIDIDGVFKSLLLLKKKKYA | |
| | ALVVEPTSDGNYVTKQELKGLDIVRRDWCDLAKDTGNFVIGQILSDQSRDTIVENIQKR | |
| | LIEIGENVLNGSVPVSQFEINKALTKDPQDYPDKKSLPHVHVALWINSQGGRKVKAGDT | |
| | VSYVICQDGSNLTASQRAYAPEQLQKQDNLTIDTQYYLAQQIHPVVARICEPIDGIDAV | |
| | LIATWLGLDPTQFRVHHYHKDEENDALLGGPAQLTDEEKYRDCERFKCPCPTCGTENIY | |
| | DNVFDGSGTDMEPSLYRCSNIDCKASPLTFTVQLSNKLIMDIRRFIKKYYDGWLICEEP | |
| | TCRNRTRHLPLQFSRTGPLCPACMKATLQPEYSDKSLYTQLCFYRYIFDAECALEKLTT | |
| | DHEKDKLKKQFFTPKVLQDYRKLKNTAEQFLSRSGYSEVNLSKLFAGCAVKS | |
| 114 | MCAFPAFPQFWDSEIPGQRHQLRSSFCVCVCTCAPVCRLDSDFKRCTVQVRLELGHRAQ | protein ENL |
| | LRKKPTTEGFTHDWMVFVRGPEQCDIQHFVEKVVFWLHDSFPKPRRVCKEPPYKVEESG | (ENL) isoform |
| | YAGFIMPIEVHFKNKEEPRKVCFTYDLFLNLEGNPPVNHLRCEKLTENNPTTEFRYKLL | X1 [Homo |
| | RAGGVMVMPEGADTVSRPSPDYPMLPTIPLSAFSDPKKTKPSHGSKDANKESSKTSKPH | sapiens] (aa) |
| | KVTKEHRERPRKDSESKSSSKELEREQAKSSKDTSRKLGEGRLPKEEKAPPPKAAFKEP | |
| | KMALKETKLESTSPKGGPPPPPPPPPRASSKRPATADSPKPSAKKQKKSSSKGSRSAPG | |
| | TSPRTSSSSSFSDKKPAKDKSSTRGEKVKAESEPREAKKALEVEESNSEDEASFKSESA | |
| | QSSPSNSSSSSDSSSDSDFEPSQNHSQGPLRSMVEDLQSEESDEDDSSSGEEAAGKINP | |
| | GRDSRLSFSDSESDNSADSSLPSREPPPPQKPPPPNSKVSGRRSPESCSKPEKILKKGT | |
| | YDKAYTDELVELHRRLMALRERNVLQQIVNLIEETGHENVINTTFDFDLFSLDETTVRK | |
| | LQSCLEAVAT | |
| 115 | MAEAPASPAPLSPLEVELDPEFEPQSRPRSCTWPLQRPELQASPAKPSGETAADSMIPE | forkhead box |
| | EEDDEDDEDGGGRAGSAMAIGGGGGSGTLGSGLLLEDSARVLAPGGQDPGSGPATAAGG | protein 03 |
| | LSGGTQALLQPQQPLPPPQPGAAGGSGQPRKCSSRRNAWGNLSYADLITRAIESSPDKR | (FOXO3) |
| | LTLSQIYEWMVRCVPYFKDKGDSNSSAGWKNSIRHNLSLHSRFMRVQNEGTGKSSWWII | [Homo sapiens] |
| | NPDGGKSGKAPRRRAVSMDNSNKYTKSRGRAAKKKAALQTAPESADDSPSQLSKWPGSP | (aa) |
| | TSRSSDELDAWTDFRSRINSNASTVSGRLSPIMASTELDEVQDDDAPLSPMLYSSSASL | |
| | SPSVSKPCTVELPRLTDMAGTMNLNDGLTENLMDDLLDNITLPPSQPSPTGGLMQRSSS | |
| | FPYTTKGSGLGSPTSSFNSTVFGPSSLNSLRQSPMQTIQENKPATFSSMSHYGNQTLQD | |
| | LLTSDSLSHSDVMMTQSDPLMSQASTAVSAQNSRRNVMLRNDPMMSFAAQPNQGSLVNQ | |
| | NLLHHQHQTQGALGGSRALSNSVSNMGLSESSSLGSAKHQQQSPVSQSMQTLSDSLSGS | |
| | SLYSTSANLPVMGHEKFPSDLDLDMENGSLECDMESIIRSELMDADGLDFNFDSLISTQ | |
| | NVVGLNVGNFTGAKQASSQSWVPG | |
| 116 | MTEAGKLPLPLPPRLDWFVHTQMGQLAQDGVPEWFHGAISREDAENLLESQPLGSFLIR | hematopoietic |
| | VSHSHVGYTLSYKAQSSCCHFMVKLLDDGTFMIPGEKVAHTSLDALVTFHQQKPIEPRR | SH2 domain- |
| | ELLTQPCRQKDPANVDYEDLFLYSNAVAEEAACPVSAPEEASPKPVLCHQSKERKPSAE | containing |
| | MNRITTKEATSSCPPKSPLGETRQKLWRSLKMLPERGQRVRQQLKSHLATVNLSSLLDV | protein (HSH2D) |
| | RRSTVISGPGTGKGSQDHSGDPTSGDRGYTDPCVATSLKSPSQPQAPKDRKVPTRKAER | isoform 1 |
| | SVSCIEVTPGDRSWHQMVVRALSSQESKPEHQGLAEPENDQLPEEYQQPPPFAPGYC | [Homo sapiens] |
| | | (aa) |
| 117 | MSGMGENTSDPSRAETRKRKECPDQLGPSPKRNTEKRNREQENKYIEELAELIFANEND | nuclear |
| | IDNFNFKPDKCAILKETVKQIRQIKEQEKAAAANIDEVQKSDVSSTGQGVIDKDALGPM | receptor |
| | MLEALDGFFFVVNLEGNVVFVSENVTQYLRYNQEELMNKSVYSILHVGDHTEFVKNLLP | coactivator 2 |
| | KSIVNGGSWSGEPPRRNSHTFNCRMLVKPLPDSEEEGHDNQEAHQKYETMQCFAVSQPK | (NCOA2) |
| | SIKEEGEDLQSCLICVARRVPMKERPVLPSSESFTTRQDLQGKITSLDTSTMRAAMKPG | isoform X1 |
| | WEDLVRRCIQKFHAQHEGESVSYAKRHHHEVLRQGLAFSQIYRFSLSDGTLVAAQTKSK | [Homo sapiens] |
| | LIRSQTTNEPQLVISLHMLHREQNVCVMNPDLTGQTMGKPLNPISSNSPAHQALCSGNP | (aa) |
| | GQDMTLSSNINFPINGPKEQMGMPMGREGGSGGMNHVSGMQATTPQGSNYALKMNSPSQ | |
| | SSPGMNPGQPTSMLSPRHRMSPGVAGSPRIPPSQFSPAGSLHSPVGVCSSTGNSHSYTN | |
| | SSLNALQALSEGHGVSLGSSLASPDLKMGNLQNSPVNMNPPPLSKMGSLDSKDCFGLYG | |
| | EPSEGTTGQAESSCHPGEQKETNDPNLPPAVSSERADGQSRLHDSKGQTKLLQLLTTKS | |
| | DQMEPSPLASSLSDTNKDSTGSLPGSGSTHGTSLKEKHKILHRLLQDSSSPVDLAKLTA | |
| | EATGKDLSQESSSTAPGSEVTIKQEPVSPKKKENALLRYLLDKDDTKDIGLPEITPKLE | |
| | RLDSKTDPASNTKLIAMKTEKEEMSFEPGDQPGSELDNLEEILDDLQNSQLPQLFPDTR | |
| | PGAPAGSVDKQAIINDLMQLTAENSPVTPVGAQKTALRISQSTENNPRPGQLGRLLPNQ | |
| | NLPLDITLQSPTGAGPFPPIRNSSPYSVIPQPGMMGNQGMIGNQGNLGNSSTGMIGNSA | |
| | SRPTMPSGEWAPQSSAVRVTCAATTSAMNRPVQGGMIRNPAASIPMRPSSQPGQRQTLQ | |
| | SQVMNIGPSELEMNMGGPQYSQQQAPPNQTAPWPESILPIDQASFASQNRQPFGSSPDD | |
| | LLCPHPAAESPSDEGALLDQLYLALRNEDGLEEIDRALGIPELVSQSQAVDPEQFSSQD | |
| | SNIMLEQKAPVFPQQYASQAQMAQGSYSPMQDPNFHTMGQRPSYATLRMQPRPGLRPTG | |
| | LVQNQPNQLRLQLQHRLQAQQNRQPLMNQISNVSNVNLTLRPGVPTQAPINAQMLAQRQ | |
| | REILNQHLRQRQMHQQQQVQQRTLMMRGQGLNMTPSMVAPSGMPATMSNPRIPQANAQQ | |
| | FPFPPNYGTGLRSPPPFTSPFSPVSPSVGSQLLSHSSLHGSQMNLANQGMIGNLGGQLG | |
| | PVRSPQVQHSTFQALSSGISQQPDPGFTGATTPQSPLMSPRMAHTQSPMMQQSQANPAY | |
| | QAPSDINGWAQGNMGGNSMFSQQSPPHFGQQANTSMYSNNMNINVSMATNTGGMSSMNQ | |
| | MTGQISMTSVTSVPTSGLSSMGPEQVNDPALRGGNLFPNQLPGMDMIKQEGDTTRKYC | |
| 118 | MSGLGENLDPLASDSRKRKLPCDTPGQGLTCSGEKRRREQESKYIEELAELISANLSDI | nuclear |
| | DNFNVKPDKCAILKETVRQIRQIKEQGKTISNDDDVQKADVSSTGQGVIDKDSLGPLLL | receptor |
| | QALDGFLFVVNRDGNIVFVSENVTQYLQYKQEDLVNTSVYNILHEEDRKDFLKNLPKST | coactivator 3 |
| | VNGVSWTNETQRQKSHTFNCRMLMKTPHDILEDINASPEMRQRYETMQCFALSQPRAMM | (NCOA3) |
| | EEGEDLQSCMICVARRITTGERTFPSNPESFITRHDLSGKVVNIDTNSLRSSMRPGFED | isoform a |
| | IIRRCIQRFFSLNDGQSWSQKRHYQEAYLNGHAETPVYRFSLADGTIVTAQTKSKLERN | [Homo sapiens] |
| | PVTNDRHGFVSTHFLQREQNGYRPNPNPVGQGIRPPMAGCNSSVGGMSMSPNQGLQMPS | (aa) |
| | SRAYGLADPSTTGQMSGARYGGSSNIASLTPGPGMQSPSSYQNNNYGLNMSSPPHGSPG | |
| | LAPNQQNIMISPRNRGSPKIASHQFSPVAGVHSPMASSGNTGNHSFSSSSLSALQAISE | |
| | GVGTSLLSTLSSPGPKLDNSPNMNITQPSKVSNQDSKSPLGFYCDQNPVESSMCQSNSR | |
| | DHLSDKESKESSVEGAENQRGPLESKGHKKLLQLLTCSSDDRGHSSLINSPLDSSCKES | |
| | SVSVTSPSGVSSSTSGGVSSTSNMHGSLLQEKHRILHKLLQNGNSPAEVAKITAEATGK | |
| | DTSSITSCGDGNVVKQEQLSPKKKENNALLRYLLDRDDPSDALSKELQPQVEGVDNKMS | |
| | QCTSSTIPSSSQEKDPKIKTETSEEGSGDLDNLDAILGDLTSSDFYNNSISSNGSHLGT | |
| | KQQVFQGTNSLGLKSSQSVQSIRPPYNRAVSLDSPVSVGSSPPVKNISAFPMLPKQPML | |
| | GGNPRMMDSQENYGSSMGGPNRNVTVTQTPSSGDWGLPNSKAGRMEPMNSNSMGRPGGD | |
| | YNTSLPRPALGGSIPTLPLRSNSIPGARPVLQQQQQMLQMRPGEIPMGMGANPYGQAAA | |
| | SNQLGSWPDGMLSMEQVSHGTQNRPLLRNSLDDLVGPPSNLEGQSDERALLDQLHTLLS | |
| | NTDATGLEEIDRALGIPELVNQGQALEPKQDAFQGQEAAVMMDQKAGLYGQTYPAQGPP | |
| | MQGGFHLQGQSPSFNSMMNQMNQQGNFPLQGMHPRANIMRPRINTPKQLRMQLQQRLQG | |
| | QQFLNQSRQALELKMENPTAGGAAVMRPMMQPQVSSQQGELNAQMVAQRSRELLSHHER | |
| | QQRVAMMMQQQQQQQQQQQQQQQQQQQQQQQQQQQQQTQAFSPPPNVTASPSMDGLLAG | |
| | PTMPQAPPQQFPYQPNYGMGQQPDPAFGRVSSPPNAMMSSRMGPSQNPMMQHPQAASIY | |
| | QSSEMKGWPSGNLARNSSFSQQQFAHQGNPAVYSMVHMNGSSGHMGQMNMNPMPMSGMP | |
| | MGPDQKYC | |
| 119 | MQLSKVKFRNQYDNDVTVWSPQGRIHQIEYAMEAVKQGSATVGLKSKTHAVLVALKRAQ | proteasome |
| | SELAAHQKKILHVDNHIGISIAGLTADARLLCNFMRQECLDSRFVEDRPLPVSRLVSLI | subunit alpha |
| | GSKTQIPTQRYGRRPYGVGLLIAGYDDMGPHIFQTCPSANYFDCRAMSIGARSQSARTY | type-1 (PSA1) |
| | LERHMSEFMECNLNELVKHGLRALRETLPAEQDLTTKNVSIGIVGKDLEFTIYDDDDVS | isoform 1 |
| | PFLEGLEERPQRKAQPAQPADEPAEKADEPMEH | [Homo sapiens] |
| | | (aa) |
| 120 | MPAENSPAPAYKVSSHGGDSGLDGLGGPGVQLGSPDKKKRKANTQGPSFPPLSEYAPPP | pygopus |
| | NPNSDHLVAANPFDDNYNTISYKPLPSSNPYLGPGYPGFGGYSTFRMPPHVPPRMSSPY | homolog 1 |
| | CGPYSLRNQPHPFPQNPLGMGFNRPHAFNFGPHDNSSFGNPSYNNALSQNVNMPNQHER | (PYGO1) |
| | QNPAENFSQIPPQNASQVSNPDLASNFVPGNNSNFTSPLESNHSFIPPPNTFGQAKAPP | isoform 1 |
| | PKQDFTQGATKNTNQNSSAHPPHLNMDDTVNQSNIELKNVNRNNAVNQENSRSSSTEAT | [Homo sapiens] |
| | NNNPANGTQNKPRQPRGAADACTTEKSNKSSLHPNRHGHSSSDPVYPCGICTNEVNDDQ | (aa) |
| | DAILCEASCQKWFHRICTGMTETAYGLLTAEASAVWGCDTCMADKDVQLMRTRETFGPS | |
| | AVGSDA | |
| 121 | MADDIDIEAMLEAPYKKDENKLSSANGHEERSKKRKKSKSRSRSHERKRSKSKERKRSR | RNA-binding |
| | DRERKKSKSRERKRSRSKERRRSRSRSRDRRFRGRYRSPYSGPKFNSAIRGKIGLPHSI | protein 39 |
| | KLSRRRSRSKSPFRKDKSPVREPIDNLTPEERDARTVFCMQLAARIRPRDLEEFFSTVG | (RBM39) |
| | KVRDVRMISDRNSRRSKGIAYVEFVDVSSVPLAIGLTGQRVLGVPIIVQASQAEKNRAA | isoform a |
| | AMANNLQKGSAGPMRLYVGSLHFNITEDMLRGIFEPFGRIESIQLMMDSETGRSKGYGF | [Homo sapiens] |
| | ITFSDSECAKKALEQLNGFELAGRPMKVGHVTERTDASSASSFLDSDELERTGIDLGTT | (aa) |
| | GRLQLMARLAEGTGLQIPPAAQQALQMSGSLAFGAVAEFSFVIDLQTRLSQQTEASALA | |
| | AAASVQPLATQCFQLSNMFNPQTEEEVGWDTEIKDDVIEECNKHGGVIHIYVDKNSAQG | |
| | NVYVKCPSIAAAIAAVNALHGRWFAGKMITAAYVPLPTYHNLFPDSMTATQLLVPSRR | |
| 122 | MPSESFCLAAQARLDSKWLKTDIQLAFTRDGLCGLWNEMVKDGEIVYTGTESTQNGELP | E3 ubiquitin- |
| | PRKDDSVEPSGTKKEDLNDKEKKDEEETPAPIYRAKSILDSWVWGKQPDVNELKECLSV | protein ligase |
| | LVKEQQALAVQSATTTLSALRLKQRLVILERYFIALNRTVFQENVKVKWKSSGISLPPV | HERC2 |
| | DKKSSRPAGKGVEGLARVGSRAALSFAFAFLRRAWRSGEDADLCSELLQESLDALRALP | (HERC2) |
| | EASLFDESTVSSVWLEVVERATRFLRSVVTGDVHGTPATKGPGSIPLQDQHLALAILLE | [Homo sapiens] |
| | LAVQRGTLSQMLSAILLLLQLWDSGAQETDNERSAQGTSAPLLPLLQRFQSIICRKDAP | (aa) |
| | HSEGDMHLLSGPLSPNESFLRYLTLPQDNELAIDLRQTAVVVMAHLDRLATPCMPPLCS | |
| | SPTSHKGSLQEVIGWGLIGWKYYANVIGPIQCEGLANLGVTQIACAEKRFLILSRNGRV | |
| | YTQAYNSDTLAPQLVQGLASRNIVKIAAHSDGHHYLALAATGEVYSWGCGDGGRLGHGD | |
| | TVPLEEPKVISAFSGKQAGKHVVHIACGSTYSAAITAEGELYTWGRGNYGRLGHGSSED | |
| | EAIPMLVAGLKGLKVIDVACGSGDAQTLAVTENGQVWSWGDGDYGKLGRGGSDGCKTPK | |
| | LIEKLQDLDVVKVRCGSQFSIALTKDGQVYSWGKGDNQRLGHGTEEHVRYPKLLEGLQG | |
| | KKVIDVAAGSTHCLALTEDSEVHSWGSNDQCQHFDTLRVTKPEPAALPGLDTKHIVGIA | |
| | CGPAQSFAWSSCSEWSIGLRVPFVVDICSMTFEQLDLLLRQVSEGMDGSADWPPPQEKE | |
| | CVAVATLNLLRLQLHAAISHQVDPEFLGLGLGSILLNSLKQTVVTLASSAGVLSTVQSA | |
| | AQAVLQSGWSVLLPTAEERARALSALLPCAVSGNEVNISPGRRFMIDLLVGSLMADGGL | |
| | ESALHAAITAEIQDIEAKKEAQKEKEIDEQEANASTFHRSRTPLDKDLINTGICESSGK | |
| | QCLPLVQLIQQLLRNIASQTVARLKDVARRISSCLDFEQHSRERSASLDLLLRFQRLLI | |
| | SKLYPGESIGQTSDISSPELMGVGSLLKKYTALLCTHIGDILPVAASIASTSWRHFAEV | |
| | AYIVEGDFTGVLLPELVVSIVLLLSKNAGLMQEAGAVPLLGGLLEHLDRENHLAPGKER | |
| | DDHEELAWPGIMESFFTGQNCRNNEEVTLIRKADLENHNKDGGFWTVIDGKVYDIKDFQ | |
| | TQSLTGNSILAQFAGEDPVVALEAALQFEDTRESMHAFCVGQYLEPDQEIVTIPDLGSL | |
| | SSPLIDTERNLGLLLGLHASYLAMSTPLSPVEIECAKWLQSSIFSGGLQTSQIHYSYNE | |
| | EKDEDHCSSPGGTPASKSRLCSHRRALGDHSQAFLQAIADNNIQDHNVKDELCQIERYC | |
| | RQCHLTTPIMFPPEHPVEEVGRLLLCCLLKHEDLGHVALSLVHAGALGIEQVKHRTLPK | |
| | SVVDVCRVVYQAKCSLIKTHQEQGRSYKEVCAPVIERLRFLFNELRPAVCNDLSIMSKF | |
| | KLLSSLPRWRRIAQKIIRERRKKRVPKKPESTDDEEKIGNEESDLEEACILPHSPINVD | |
| | KRPIAIKSPKDKWQPLLSTVTGVHKYKWLKQNVQGLYPQSPLLSTIAEFALKEEPVDVE | |
| | KMRKCLLKQLERAEVRLEGIDTILKLASKNFLLPSVQYAMFCGWQRLIPEGIDIGEPLT | |
| | DCLKDVDLIPPENRMLLEVTFGKLYAWAVQNIRNVLMDASAKFKELGIQPVPLQTITNE | |
| | NPSGPSLGTIPQARFLLVMLSMLTLQHGANNLDLLLNSGMLALTQTALRLIGPSCDNVE | |
| | EDMNASAQGASATVLEETRKETAPVQLPVSGPELAAMMKIGTRVMRGVDWKWGDQDGPP | |
| | PGLGRVIGELGEDGWIRVQWDTGSTNSYRMGKEGKYDLKLAELPAAAQPSAEDSDTEDD | |
| | SEAEQTERNIHPTAMMFTSTINLLQTLCLSAGVHAEIMQSEATKTLCGLLRMLVESGTT | |
| | DKTSSPNRLVYREQHRSWCTLGFVRSIALTPQVCGALSSPQWITLLMKVVEGHAPFTAT | |
| | SLQRQILAVHLLQAVLPSWDKTERARDMKCLVEKLFDFLGSLLTTCSSDVPLLRESTLR | |
| | RRRVRPQASLTATHSSTLAEEVVALLRTLHSLTQWNGLINKYINSQLRSITHSFVGRPS | |
| | EGAQLEDYFPDSENPEVGGLMAVLAVIGGIDGRLRLGGQVMHDEFGEGTVTRITPKGKI | |
| | TVQFSDMRTCRVCPLNQLKPLPAVAFNVNNLPFTEPMLSVWAQLVNLAGSKLEKHKIKK | |
| | STKQAFAGQVDLDLLRCQQLKLYILKAGRALLSHQDKLRQILSQPAVQETGTVHTDDGA | |
| | VVSPDLGDMSPEGPQPPMILLQQLLASATQPSPVKAIFDKQELEAAALAVCQCLAVEST | |
| | HPSSPGFEDCSSSEATTPVAVQHIRPARVKRRKQSPVPALPIVVQLMEMGFSRRNIEFA | |
| | LKSLTGASGNASSLPGVEALVGWLLDHSDIQVTELSDADTVSDEYSDEEVVEDVDDAAY | |
| | SMSTGAVVTESQTYKKRADFLSNDDYAVYVRENIQVGMMVRCCRAYEEVCEGDVGKVIK | |
| | LDRDGLHDLNVQCDWQQKGGTYWVRYIHVELIGYPPPSSSSHIKIGDKVRVKASVTTPK | |
| | YKWGSVTHQSVGVVKAFSANGKDIIVDFPQQSHWTGLLSEMELVPSIHPGVTCDGCQMF | |
| | PINGSRFKCRNCDDFDFCETCFKTKKHNTRHTFGRINEPGQSAVFCGRSGKQLKRCHSS | |
| | QPGMLLDSWSRMVKSLNVSSSVNQASRLIDGSEPCWQSSGSQGKHWIRLEIFPDVLVHR | |
| | LKMIVDPADSSYMPSLVVVSGGNSLNNLIELKTININPSDTTVPLLNDCTEYHRYIEIA | |
| | IKQCRSSGIDCKIHGLILLGRIRAEEEDLAAVPFLASDNEEEEDEKGNSGSLIRKKAAG | |
| | LESAATIRTKVFVWGLNDKDQLGGLKGSKIKVPSFSETLSALNVVQVAGGSKSLFAVTV | |
| | EGKVYACGEATNGRLGLGISSGTVPIPRQITALSSYVVKKVAVHSGGRHATALTVDGKV | |
| | FSWGEGDDGKLGHFSRMNCDKPRLIEALKTKRIRDIACGSSHSAALTSSGELYTWGLGE | |
| | YGRLGHGDNTTQLKPKMVKVLLGHRVIQVACGSRDAQTLALTDEGLVESWGDGDFGKLG | |
| | RGGSEGCNIPQNIERLNGQGVCQIECGAQFSLALTKSGVVWTWGKGDYFRLGHGSDVHV | |
| | RKPQVVEGLRGKKIVHVAVGALHCLAVTDSGQVYAWGDNDHGQQGNGTTTVNRKPTLVQ | |
| | GLEGQKITRVACGSSHSVAWTTVDVATPSVHEPVLFQTARDPLGASYLGVPSDADSSAA | |
| | SNKISGASNSKPNRPSLAKILLSLDGNLAKQQALSHILTALQIMYARDAVVGALMPAAM | |
| | IAPVECPSFSSAAPSDASAMASPMNGEECMLAVDIEDRLSPNPWQEKREIVSSEDAVTP | |
| | SAVTPSAPSASARPFIPVTDDLGAASIIAETMTKTKEDVESQNKAAGPEPQALDEFTSL | |
| | LIADDTRVVVDLLKLSVCSRAGDRGRDVLSAVLSGMGTAYPQVADMLLELCVTELEDVA | |
| | TDSQSGRLSSQPVVVESSHPYTDDTSTSGTVKIPGAEGLRVEFDRQCSTERRHDPLTVM | |
| | DGVNRIVSVRSGREWSDWSSELRIPGDELKWKFISDGSVNGWGWRFTVYPIMPAAGPKE | |
| | LLSDRCVLSCPSMDLVTCLLDFRLNLASNRSIVPRLAASLAACAQLSALAASHRMWALQ | |
| | RLRKLLTTEFGQSININRLLGENDGETRALSFTGSALAALVKGLPEALQRQFEYEDPIV | |
| | RGGKQLLHSPFFKVLVALACDLELDTLPCCAETHKWAWERRYCMASRVAVALDKRTPLP | |
| | RLFLDEVAKKIRELMADSENMDVLHESHDIFKREQDEQLVQWMNRRPDDWTLSAGGSGT | |
| | IYGWGHNHRGQLGGIEGAKVKVPTPCEALATLRPVQLIGGEQTLFAVTADGKLYATGYG | |
| | AGGRLGIGGTESVSTPTLLESIQHVFIKKVAVNSGGKHCLALSSEGEVYSWGEAEDGKL | |
| | GHGNRSPCDRPRVIESLRGIEVVDVAAGGAHSACVTAAGDLYTWGKGRYGRLGHSDSED | |
| | QLKPKLVEALQGHRVVDIACGSGDAQTLCLTDDDTVWSWGDGDYGKLGRGGSDGCKVPM | |
| | KIDSLTGLGVVKVECGSQFSVALTKSGAVYTWGKGDYHRLGHGSDDHVRRPRQVQGLQG | |
| | KKVIAIATGSLHCVCCTEDGEVYTWGDNDEGQLGDGTTNAIQRPRLVAALQGKKVNRVA | |
| | CGSAHTLAWSTSKPASAGKLPAQVPMEYNHLQEIPIIALRNRLLLLHHLSELFCPCIPM | |
| | FDLEGSLDETGLGPSVGFDTLRGILISQGKEAAFRKVVQATMVRDRQHGPVVELNRIQV | |
| | KRSRSKGGLAGPDGTKSVFGQMCAKMSSFGPDSLLLPHRVWKVKFVGESVDDCGGGYSE | |
| | SIAEICEELQNGLTPLLIVTPNGRDESGANRDCYLLSPAARAPVHSSMFRFLGVLLGIA | |
| | IRTGSPLSLNLAEPVWKQLAGMSLTIADLSEVDKDFIPGLMYIRDNEATSEEFEAMSLP | |
| | FTVPSASGQDIQLSSKHTHITLDNRAEYVRLAINYRLHEFDEQVAAVREGMARVVPVPL | |
| | LSLFTGYELETMVCGSPDIPLHLLKSVATYKGIEPSASLIQWFWEVMESFSNTERSLFL | |
| | RFVWGRTRLPRTIADFRGRDFVIQVLDKYNPPDHFLPESYTCFFLLKLPRYSCKQVLEE | |
| | KLKYAIHFCKSIDTDDYARIALTGEPAADDSSDDSDNEDVDSFASDSTQDYLTGH | |
| 123 | MLWWEEVEDCYEREDVQKKTFTKWVNAQFSKFGKQHIENLFSDLQDGRRLLDLLEGLTG | dystrophin |
| | QKLPKEKGSTRVHALNNVNKALRVLQNNNVDLVNIGSTDIVDGNHKLILGLIWNIILHW | (DMD) isoform |
| | QVKNVMKNIMAGLQQTNSEKILLSWVRQSTRNYPQVNVINFTTSWSDGLALNALIHSHR | X1 [Homo |
| | PDLFDWNSVVCQQSATQRLEHAFNIARYQLGIEKLLDPEDVDTTYPDKKSILMYITSLF | sapiens] (aa) |
| | QVLPQQVSIEAIQEVEMLPRPPKVTKEEHFQLHHQMHYSQQITVSLAQGYERTSSPKPR | |
| | FKSYAYTQAAYVTTSDPTRSPFPSQHLEAPEDKSFGSSLMESEVNLDRYQTALEEVLSW | |
| | LLSAEDTLQAQGEISNDVEVVKDQFHTHEGYMMDLTAHQGRVGNILQLGSKLIGTGKLS | |
| | EDEETEVQEQMNLLNSRWECLRVASMEKQSNLHRVLMDLQNQKLKELNDWLTKTEERTR | |
| | KMEEEPLGPDLEDLKRQVQQHKVLQEDLEQEQVRVNSLTHMVVVVDESSGDHATAALEE | |
| | QLKVLGDRWANICRWTEDRWVLLQDILLKWQRLTEEQCLFSAWLSEKEDAVNKIHTTGF | |
| | KDQNEMLSSLQKLAVLKADLEKKKQSMGKLYSLKQDLLSTLKNKSVTQKTEAWLDNFAR | |
| | CWDNLVQKLEKSTAQISQAVTTTQPSLTQTTVMETVTTVTTREQILVKHAQEELPPPPP | |
| | QKKRQITVDSEIRKRLDVDITELHSWITRSEAVLQSPEFAIFRKEGNFSDLKEKVNAIE | |
| | REKAEKFRKLQDASRSAQALVEQMVNEGVNADSIKQASEQLNSRWIEFCQLLSERLNWL | |
| | EYQNNIIAFYNQLQQLEQMTTTAENWLKIQPTTPSEPTAIKSQLKICKDEVNRLSDLQP | |
| | QIERLKIQSIALKEKGQGPMFLDADFVAFTNHFKQVFSDVQAREKELQTIFDTLPPMRY | |
| | QETMSAIRTWVQQSETKLSIPQLSVTDYEIMEQRLGELQALQSSLQEQQSGLYYLSTTV | |
| | KEMSKKAPSEISRKYQSEFEEIEGRWKKLSSQLVEHCQKLEEQMNKLRKIQNHIQTLKK | |
| | WMAEVDVFLKEEWPALGDSEILKKQLKQCRLLVSDIQTIQPSLNSVNEGGQKIKNEAEP | |
| | EFASRLETELKELNTQWDHMCQQVYARKEALKGGLEKTVSLQKDLSEMHEWMTQAEEEY | |
| | LERDFEYKTPDELQKAVEEMKRAKEEAQQKEAKVKLLTESVNSVIAQAPPVAQEALKKE | |
| | LETLTTNYQWLCTRLNGKCKTLEEVWACWHELLSYLEKANKWLNEVEFKLKTTENIPGG | |
| | AEEISEVLDSLENLMRHSEDNPNQIRILAQTLTDGGVMDELINEELETENSRWRELHEE | |
| | AVRRQKLLEQSIQSAQETEKSLHLIQESLTFIDKQLAAYIADKVDAAQMPQEAQKIQSD | |
| | LTSHEISLEEMKKHNQGKEAAQRVLSQIDVAQKKLQDVSMKFRLFQKPANFEQRLQESK | |
| | MILDEVKMHLPALETKSVEQEVVQSQLNHCVNLYKSLSEVKSEVEMVIKTGRQIVQKKQ | |
| | TENPKELDERVTALKLHYNELGAKVTERKQQLEKCLKLSRKMRKEMNVLTEWLAATDME | |
| | LTKRSAVEGMPSNLDSEVAWGKATQKEIEKQKVHLKSITEVGEALKTVLGKKETLVEDK | |
| | LSLLNSNWIAVTSRAEEWLNLLLEYQKHMETFDQNVDHITKWIIQADTLLDESEKKKPQ | |
| | QKEDVLKRLKAELNDIRPKVDSTRDQAANLMANRGDHCRKLVEPQISELNHRFAAISHR | |
| | IKTGKASIPLKELEQFNSDIQKLLEPLEAEIQQGVNLKEEDENKDMNEDNEGTVKELLQ | |
| | RGDNLQQRITDERKREEIKIKQQLLQTKHNALKDLRSQRRKKALEISHQWYQYKRQADD | |
| | LLKCLDDIEKKLASLPEPRDERKIKEIDRELQKKKEELNAVRRQAEGLSEDGAAMAVEP | |
| | TQIQLSKRWREIESKFAQFRRLNFAQIHTVREETMMVMTEDMPLEISYVPSTYLTEITH | |
| | VSQALLEVEQLLNAPDLCAKDFEDLFKQEESLKNIKDSLQQSSGRIDIIHSKKTAALQS | |
| | ATPVERVKLQEALSQLDFQWEKVNKMYKDRQGRFDRSVEKWRRFHYDIKIFNQWLTEAE | |
| | QFLRKTQIPENWEHAKYKWYLKELQDGIGQRQTVVRTLNATGEEIIQQSSKTDASILQE | |
| | KLGSLNLRWQEVCKQLSDRKKRLEEQKNILSEFQRDLNEFVLWLEEADNIASIPLEPGK | |
| | EQQLKEKLEQVKLLVEELPLRQGILKQLNETGGPVLVSAPISPEEQDKLENKLKQTNLQ | |
| | WIKVSRALPEKQGEIEAQIKDLGQLEKKLEDLEEQLNHLLLWLSPIRNQLEIYNQPNQE | |
| | GPFDVKETEIAVQAKQPDVEEILSKGQHLYKEKPATQPVKRKLEDLSSEWKAVNRLLQE | |
| | LRAKQPDLAPGLTTIGASPTQTVTLVTQPVVTKETAISKLEMPSSLMLEVPALADENRA | |
| | WTELTDWLSLLDQVIKSQRVMVGDLEDINEMIIKQKATMQDLEQRRPQLEELITAAQNL | |
| | KNKTSNQEARTIITDRIERIQNQWDEVQEHLQNRRQQLNEMLKDSTQWLEAKEEAEQVL | |
| | GQARAKLESWKEGPYTVDAIQKKITETKQLAKDLRQWQTNVDVANDLALKLLRDYSADD | |
| | TRKVHMITENINASWRSIHKRVSEREAALEETHRLLQQFPLDLEKFLAWLTEAETTANV | |
| | LQDATRKERLLEDSKGVKELMKQWQDLQGEIEAHTDVYHNLDENSQKILRSLEGSDDAV | |
| | LLQRRLDNMNFKWSELRKKSLNIRSHLEASSDQWKRLHLSLQELLVWLQLKDDELSRQA | |
| | PIGGDFPAVQKQNDVHRAFKRELKTKEPVIMSTLETVRIFLTEQPLEGLEKLYQEPREL | |
| | PPEERAQNVTRLLRKQAEEVNTEWEKLNLHSADWQRKIDETLERLRELQEATDELDLKL | |
| | RQAEVIKGSWQPVGDLLIDSLQDHLEKVKALRGEIAPLKENVSHVNDLARQLTTLGIQL | |
| | SPYNLSTLEDLNTRWKLLQVAVEDRVRQLHEAHRDFGPASQHELSTSVQGPWERAISPN | |
| | KVPYYINHETQTTCWDHPKMTELYQSLADLNNVRFSAYRTAMKLRRLQKALCLDLLSLS | |
| | AACDALDQHNLKQNDQPMDILQIINCLTTIYDRLEQEHNNLVNVPLCVDMCLNWLLNVY | |
| | DTGRTGRIRVLSFKTGIISLCKAHLEDKYRYLFKQVASSTGFCDQRRLGLLLHDSIQIP | |
| | RQLGEVASFGGSNIEPSVRSCFQFANNKPEIEAALFLDWMRLEPQSMVWLPVLHRVAAA | |
| | ETAKHQAKCNICKECPIIGFRYRSLKHENYDICQSCFFSGRVAKGHKMHYPMVEYCTPT | |
| | TSGEDVRDFAKVLKNKFRTKRYFAKHPRMGYLPVQTVLEGDNMETPVTLINFWPVDSAP | |
| | ASSPQLSHDDTHSRIEHYASRLAEMENSNGSYLNDSISPNESIDDEHLLIQHYCQSLNQ | |
| | DSPLSQPRSPAQILISLESEERGELERILADLEEENRNLQAEYDRLKQQHEHKGLSPLP | |
| | SPPEMMPTSPQSPRDAELIAEAKLLRQHKGRLEARMQILEDHNKQLESQLHRLRQLLEQ | |
| | PQAEAKVNGTTVSSPSTSLQRSDSSQPMLLRVVGSQTSDSMGEEDLLSPPQDTSTGLEE | |
| | VMEQLNNSFPSSRGHNVGSLFHMADDLGRAMESLVSVMTDEEGAE | |
| 124 | MPALRPALLWALLALWLCCAAPAHALQCRDGYEPCVNEGMCVTYHNGTGYCKCPEGFLG | NOTCH 2 |
| | EYCQHRDPCEKNRCQNGGTCVAQAMLGKATCRCASGFTGEDCQYSTSHPCFVSRPCLNG | (NOTC2) |
| | GTCHMLSRDTYECTCQVGFTGKECQWTDACLSHPCANGSTCTTVANQFSCKCLTGFTGQ | [Homo sapiens] |
| | KCETDVNECDIPGHCQHGGTCLNLPGSYQCQCPQGFTGQYCDSLYVPCAPSPCVNGGTC | (aa) |
| | RQTGDFTFECNCLPGFEGSTCERNIDDCPNHRCQNGGVCVDGVNTYNCRCPPQWTGQFC | |
| | TEDVDECLLQPNACQNGGTCANRNGGYGCVCVNGWSGDDCSENIDDCAFASCTPGSTCI | |
| | DRVASFSCMCPEGKAGLLCHLDDACISNPCHKGALCDTNPLNGQYICTCPQGYKGADCT | |
| | EDVDECAMANSNPCEHAGKCVNTDGAFHCECLKGYAGPRCEMDINECHSDPCQNDATCL | |
| | DKIGGFTCLCMPGFKGVHCELEINECQSNPCVNNGQCVDKVNRFQCLCPPGFTGPVCQI | |
| | DIDDCSSTPCLNGAKCIDHPNGYECQCATGFTGVLCEENIDNCDPDPCHHGQCQDGIDS | |
| | YTCICNPGYMGAICSDQIDECYSSPCLNDGRCIDLVNGYQCNCQPGTSGVNCEINFDDC | |
| | ASNPCIHGICMDGINRYSCVCSPGFTGQRCNIDIDECASNPCRKGATCINGVNGFRCIC | |
| | PEGPHHPSCYSQVNECLSNPCIHGNCTGGLSGYKCLCDAGWVGINCEVDKNECLSNPCQ | |
| | NGGTCDNLVNGYRCTCKKGFKGYNCQVNIDECASNPCLNQGTCEDDISGYTCHCVLPYT | |
| | GKNCQTVLAPCSPNPCENAAVCKESPNFESYTCLCAPGWQGQRCTIDIDECISKPCMNH | |
| | GLCHNTQGSYMCECPPGFSGMDCEEDIDDCLANPCQNGGSCMDGVNTFSCLCLPGFTGD | |
| | KCQTDMNECLSEPCKNGGTCSDYVNSYTCKCQAGEDGVHCENNINECTESSCENGGTCV | |
| | DGINSFSCLCPVGFTGSFCLHEINECSSHPCLNEGTCVDGLGTYRCSCPLGYTGKNCQT | |
| | LVNLCSRSPCKNKGTCVQKKAESQCLCPSGWAGAYCDVPNVSCDIAASRRGVLVEHLCQ | |
| | HSGVCINAGNTHYCQCPLGYTGSYCEEQLDECASNPCQHGATCSDFIGGYRCECVPGYQ | |
| | GVNCEYEVDECQNQPCQNGGTCIDLVNHFKCSCPPGTRGLLCEENIDDCARGPHCLNGG | |
| | QCMDRIGGYSCRCLPGFAGERCEGDINECLSNPCSSEGSLDCIQLINDYLCVCRSAFTG | |
| | RHCETFVDVCPQMPCLNGGTCAVASNMPDGFICRCPPGFSGARCQSSCGQVKCRKGEQC | |
| | VHTASGPRCFCPSPRDCESGCASSPCQHGGSCHPQRQPPYYSCQCAPPFSGSRCELYTA | |
| | PPSTPPATCLSQYCADKARDGVCDEACNSHACQWDGGDCSLTMENPWANCSSPLPCWDY | |
| | INNQCDELCNTVECLFDNFECQGNSKTCKYDKYCADHFKDNHCNQGCNSEECGWDGLDC | |
| | AADQPENLAEGTLVIVVLMPPEQLLQDARSFLRALGTLLHTNLRIKRDSQGELMVYPYY | |
| | GEKSAAMKKQRMTRRSLPGEQEQEVAGSKVFLEIDNRQCVQDSDHCFKNTDAAAALLAS | |
| | HAIQGTLSYPLVSVVSESLTPERTQLLYLLAVAVVIILFIILLGVIMAKRKRKHGSLWL | |
| | PEGFTLRRDASNHKRREPVGQDAVGLKNLSVQVSEANLIGTGTSEHWVDDEGPQPKKVK | |
| | AEDEALLSEEDDPIDRRPWTQQHLEAADIRRTPSLALTPPQAEQEVDVLDVNVRGPDGC | |
| | TPLMLASLRGGSSDLSDEDEDAEDSSANIITDLVYQGASLQAQTDRTGEMALHLAARYS | |
| | RADAAKRLLDAGADANAQDNMGRCPLHAAVAADAQGVFQILIRNRVTDLDARMNDGTTP | |
| | LILAARLAVEGMVAELINCQADVNAVDDHGKSALHWAAAVNNVEATLLLLKNGANRDMQ | |
| | DNKEETPLFLAAREGSYEAAKILLDHFANRDITDHMDRLPRDVARDRMHHDIVRLLDEY | |
| | NVTPSPPGTVLTSALSPVICGPNRSFLSLKHTPMGKKSRRPSAKSTMPTSLPNLAKEAK | |
| | DAKGSRRKKSLSEKVQLSESSVTLSPVDSLESPHTYVSDTTSSPMITSPGILQASPNPM | |
| | LATAAPPAPVHAQHALSFSNLHEMQPLAHGASTVLPSVSQLLSHHHIVSPGSGSAGSLS | |
| | RLHPVPVPADWMNRMEVNETQYNEMFGMVLAPAEGTHPGIAPQSRPPEGKHITTPREPL | |
| | PPIVTFQLIPKGSIAQPAGAPQPQSTCPPAVAGPLPTMYQIPEMARLPSVAFPTAMMPQ | |
| | QDGQVAQTILPAYHPFPASVGKYPTPPSQHSYASSNAAERTPSHSGHLQGEHPYLTPSP | |
| | ESPDQWSSSSPHSASDWSDVTTSPTPGGAGGGQRGPGTHMSEPPHNNMQVYA | |
| 125 | MAENVVEPGPPSAKRPKLSSPALSASASDGTDFGSLFDLEHDLPDELINSTELGLINGG | p300 (aa) |
| | DINQLQTSLGMVQDAASKHKQLSELLRSGSSPNLNMGVGGPGQVMASQAQQSSPGLGLI | |
| | NSMVKSPMTQAGLTSPNMGMGTSGPNQGPTQSTGMMNSPVNQPAMGMNTGMNAGMNPGM | |
| | LAAGNGQGIMPNQVMNGSIGAGRGRQNMQYPNPGMGSAGNLLTEPLQQGSPQMGGQTGL | |
| | RGPQPLKMGMMNNPNPYGSPYTQNPGQQIGASGLGLQIQTKTVLSNNLSPFAMDKKAVP | |
| | GGGMPNMGQQPAPQVQQPGLVTPVAQGMGSGAHTADPEKRKLIQQQLVLLLHAHKCQRR | |
| | EQANGEVRQCNLPHCRTMKNVLNHMTHCQSGKSCQVAHCASSRQIISHWKNCTRHDCPV | |
| | CLPLKNAGDKRNQQPILTGAPVGLGNPSSLGVGQQSAPNLSTVSQIDPSSIERAYAALG | |
| | LPYQVNQMPTQPQVQAKNQQNQQPGQSPQGMRPMSNMSASPMGVNGGVGVQTPSLLSDS | |
| | MLHSAINSQNPMMSENASVPSMGPMPTAAQPSTTGIRKQWHEDITQDLRNHLVHKLVQA | |
| | IFPTPDPAALKDRRMENLVAYARKVEGDMYESANNRAEYYHLLAEKIYKIQKELEEKRR | |
| | TRLQKQNMLPNAAGMVPVSMNPGPNMGQPQPGMTSNGPLPDPSMIRGSVPNQMMPRITP | |
| | QSGLNQFGQMSMAQPPIVPRQTPPLQHHGQLAQPGALNPPMGYGPRMQQPSNQGQFLPQ | |
| | TQFPSQGMNVTNIPLAPSSGQAPVSQAQMSSSSCPVNSPIMPPGSQGSHIHCPQLPQPA | |
| | LHQNSPSPVPSRTPTPHHTPPSIGAQQPPATTIPAPVPTPPAMPPGPQSQALHPPPRQT | |
| | PTPPTTQLPQQVQPSLPAAPSADQPQQQPRSQQSTAASVPTPTAPLLPPQPATPLSQPA | |
| | VSIEGQVSNPPSTSSTEVNSQAIAEKQPSQEVKMEAKMEVDQPEPADTQPEDISESKVE | |
| | DCKMESTETEERSTELKTEIKEEEDQPSTSATQSSPAPGQSKKKIFKPEELRQALMPTL | |
| | EALYRQDPESLPFRQPVDPQLLGIPDYFDIVKSPMDLSTIKRKLDTGQYQEPWQYVDDI | |
| | WLMFNNAWLYNRKTSRVYKYCSKLSEVFEQEIDPVMQSLGYCCGRKLEFSPQTLCCYGK | |
| | QLCTIPRDATYYSYQNRYHFCEKCFNEIQGESVSLGDDPSQPQTTINKEQFSKRKNDTL | |
| | DPELFVECTECGRKMHQICVLHHEIIWPAGFVCDGCLKKSARTRKENKFSAKRLPSTRL | |
| | GTFLENRVNDFLRRQNHPESGEVTVRVVHASDKTVEVKPGMKARFVDSGEMAESFPYRT | |
| | KALFAFEEIDGVDLCFFGMHVQEYGSDCPPPNQRRVYISYLDSVHFFRPKCLRTAVYHE | |
| | ILIGYLEYVKKLGYTTGHIWACPPSEGDDYIFHCHPPDQKIPKPKRLQEWYKKMLDKAV | |
| | SERIVHDYKDIFKQATEDRLTSAKELPYFEGDFWPNVLEESIKELEQEEEERKREENTS | |
| | NESTDVTKGDSKNAKKKNNKKTSKNKSSLSRGNKKKPGMPNVSNDLSQKLYATMEKHKE | |
| | VFFVIRLIAGPAANSLPPIVDPDPLIPCDLMDGRDAFLTLARDKHLEFSSLRRAQWSTM | |
| | CMLVELHTQSQDRFVYTCNECKHHVETRWHCTVCEDYDLCITCYNTKNHDHKMEKLGLG | |
| | LDDESNNQQAAATQSPGDSRRLSIQRCIQSLVHACQCRNANCSLPSCQKMKRVVQHTKG | |
| | CKRKTNGGCPICKQLIALCCYHAKHCQENKCPVPFCLNIKQKLRQQQLQHRLQQAQMLR | |
| | RRMASMQRTGVVGQQQGLPSPTPATPTTPTGQQPTTPQTPQPTSQPQPTPPNSMPPYLP | |
| | RTQAAGPVSQGKAAGQVTPPTPPQTAQPPLPGPPPAAVEMAMQIQRAAETQRQMAHVQI | |
| | FQRPIQHQMPPMTPMAPMGMNPPPMTRGPSGHLEPGMGPTGMQQQPPWSQGGLPQPQQL | |
| | QSGMPRPAMMSVAQHGQPLNMAPQPGLGQVGISPLKPGTVSQQALQNLLRTLRSPSSPL | |
| | QQQQVLSILHANPQLLAAFIKQRAAKYANSNPQPIPGQPGMPQGQPGLQPPTMPGQQGV | |
| | HSNPAMQNMNPMQAGVQRAGLPQQQPQQQLQPPMGGMSPQAQQMNMNHNTMPSQFRDIL | |
| | RRQQMMQQQQQQGAGPGIGPGMANHNQFQQPQGVGYPPQQQQRMQHHMQQMQQGNMGQI | |
| | GQLPQALGAEAGASLQAYQQRLLQQQMGSPVQPNPMSPQQHMLPNQAQSPHLQGQQIPN | |
| | SLSNQVRSPQPVPSPRPQSQPPHSSPSPRMQPQPSPHHVSPQTSSPHPGLVAAQANPME | |
| | QGHFASPDQNSMLSQLASNPGMANLHGASATDLGLSTDNSDLNSNLSQSTLDIH | |
| 126 | KRNYILGLDIGITSVGYGIIDYETRDVIDAGVRLFKEANVENNEGRRSKRGARRLKRRR | WT Sa Cas9 |
| | RHRIQRVKKLLFDYNLLTDHSELSGINPYEARVKGLSQKLSEEEFSAALLHLAKRRGVH | (aa) |
| | NVNEVEEDTGNELSTKEQISRNSKALEEKYVAELQLERLKKDGEVRGSINRFKTSDYVK | |
| | EAKQLLKVQKAYHQLDQSFIDTYIDLLETRRTYYEGPGEGSPFGWKDIKEWYEMLMGHC | |
| | TYFPEELRSVKYAYNADLYNALNDLNNLVITRDENEKLEYYEKFQIIENVFKQKKKPTL | |
| | KQIAKEILVNEEDIKGYRVTSTGKPEFTNLKVYHDIKDITARKEIIENAELLDQIAKIL | |
| | TIYQSSEDIQEELTNLNSELTQEEIEQISNLKGYTGTHNLSLKAINLILDELWHINDNQ | |
| | IAIFNRLKLVPKKVDLSQQKEIPTTLVDDFILSPVVKRSFIQSIKVINAIIKKYGLPND | |
| | IIIELAREKNSKDAQKMINEMQKRNRQTNERIEEIIRTTGKENAKYLIEKIKLHDMQEG | |
| | KCLYSLEAIPLEDLLNNPFNYEVDHIIPRSVSFDNSFNNKVLVKQEENSKKGNRTPFQY | |
| | LSSSDSKISYETFKKHILNLAKGKGRISKTKKEYLLEERDINRFSVQKDFINRNLVDTR | |
| | YATRGLMNLLRSYFRVNNLDVKVKSINGGFTSFLRRKWKFKKERNKGYKHHAEDALIIA | |
| | NADFIFKEWKKLDKAKKVMENQMFEEKQAESMPEIETEQEYKEIFITPHQIKHIKDEKD | |
| | YKYSHRVDKKPNRELINDTLYSTRKDDKGNTLIVNNLNGLYDKDNDKLKKLINKSPEKL | |
| | LMYHHDPQTYQKLKLIMEQYGDEKNPLYKYYEETGNYLTKYSKKDNGPVIKKIKYYGNK | |
| | LNAHLDITDDYPNSRNKVVKLSLKPYRFDVYLDNGVYKFVTVKNLDVIKKENYYEVNSK | |
| | CYEEAKKLKKISNQAEFIASFYNNDLIKINGELYRVIGVNNDLLNRIEVNMIDITYREY | |
| | LENMNDKRPPRIIKTIASKTQSIKKYSTDILGNLYEVKSKKHPQIIKKG | |
| 127 | DKKYSIGLDIGTNSVGWAVITDEYKVPSKKFKVLGNTDRHSIKKNLIGALLFDSGETAE | WT Sp Cas9 |
| | ATRLKRTARRRYTRRKNRICYLQEIFSNEMAKVDDSFFHRLEESFLVEEDKKHERHPIF | (aa) |
| | GNIVDEVAYHEKYPTIYHLRKKLVDSTDKADLRLIYLALAHMIKFRGHFLIEGDLNPDN | |
| | SDVDKLFIQLVQTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGDQYADLFLAAKNL | |
| | SDAILLSDILRVNTEITKAPLSASMIKRYDEHHQDLTLLKALVRQQLPEKYKEIFFDQS | |
| | KNGYAGYIDGGASQEEFYKFIKPILEKMDGTEELLVKLNREDLLRKQRTFDNGSIPHQI | |
| | HLGELHAILRRQEDFYPFLKDNREKIEKILTFRIPYYVGPLARGNSRFAWMTRKSEETI | |
| | TPWNFEEVVDKGASAQSFIERMTNFDKNLPNEKVLPKHSLLYEYFTVYNELTKVKYVTE | |
| | GMRKPAFLSGEQKKAIVDLLFKTNRKVTVKQLKEDYFKKIECFDSVEISGVEDRENASL | |
| | GTYHDLLKIIKDKDFLDNEENEDILEDIVLTLTLFEDREMIEERLKTYAHLEDDKVMKQ | |
| | LKRRRYTGWGRLSRKLINGIRDKQSGKTILDFLKSDGFANRNFMQLIHDDSLTFKEDIQ | |
| | KAQVSGQGDSLHEHIANLAGSPAIKKGILQTVKVVDELVKVMGRHKPENIVIEMARENQ | |
| | TTQKGQKNSRERMKRIEEGIKELGSQILKEHPVENTQLQNEKLYLYYLQNGRDMYVDQE | |
| | LDINRLSDYDVDHIVPQSFLKDDSIDNKVLTRSDKNRGKSDNVPSEEVVKKMKNYWRQL | |
| | LNAKLITQRKFDNLTKAERGGLSELDKAGFIKRQLVETRQITKHVAQILDSRMNTKYDE | |
| | NDKLIREVKVITLKSKLVSDFRKDFQFYKVREINNYHHAHDAYLNAVVGTALIKKYPKL | |
| | ESEFVYGDYKVYDVRKMIAKSEQEIGKATAKYFFYSNIMNFFKTEITLANGEIRKRPLI | |
| | ETNGETGEIVWDKGRDFATVRKVLSMPQVNIVKKTEVQTGGFSKESILPKRNSDKLIAR | |
| | KKDWDPKKYGGFDSPTVAYSVLVVAKVEKGKSKKLKSVKELLGITIMERSSFEKNPIDE | |
| | LEAKGYKEVKKDLIIKLPKYSLFELENGRKRMLASAGELQKGNELALPSKYVNFLYLAS | |
| | HYEKLKGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGLYET | |
| | RIDLSQLGGD | |
| 128 | GACTACAAAGACCATGACGGTGATTATAAAGATCATGACATCGATTACAAGGATGACGA | FLAG epitope |
| | TGACAAG | tag (nt) |
| 129 | DYKDHDGDYKDHDIDYKDDDDK | FLAG epitope |
| | | tag (aa) |
| 130 | GCAACAAACTTCTCTCTGCTGAAACAAGCCGGAGATGTCGAAGAGAATCCTGGACCG | P2A sequence |
| | | (nt) |
| 131 | ATNFSLLKQAGDVEENPGP | P2A sequence |
| | | (aa) |
| 132 | ATGACCGAGTACAAGCCCACGGTGCGCCTCGCCACCCGCGACGACGTCCCCAGGGCCGT | Puromycin |
| | ACGCACCCTCGCCGCCGCGTTCGCCGACTACCCCGCCACGCGCCACACCGTCGATCCGG | resistance gene |
| | ACCGCCACATCGAGCGGGTCACCGAGCTGCAAGAACTCTTCCTCACGCGCGTCGGGCTC | (nt) |
| | GACATCGGCAAGGTGTGGGTCGCGGACGACGGCGCCGCGGTGGCGGTCTGGACCACGCC | |
| | GGAGAGCGTCGAAGCGGGGGCGGTGTTCGCCGAGATCGGCCCGCGCATGGCCGAGTTGA | |
| | GCGGTTCCCGGCTGGCCGCGCAGCAACAGATGGAAGGCCTCCTGGCGCCGCACCGGCCC | |
| | AAGGAGCCCGCGTGGTTCCTGGCCACCGTCGGCGTGTCGCCCGACCACCAGGGCAAGGG | |
| | TCTGGGCAGCGCCGTCGTGCTCCCCGGAGTGGAGGCGGCCGAGCGCGCCGGGGTGCCCG | |
| | CCTTCCTGGAGACCTCCGCGCCCCGCAACCTCCCCTTCTACGAGCGGCTCGGCTTCACC | |
| | GTCACCGCCGACGTCGAGGTGCCCGAAGGACCGCGCACCTGGTGCATGACCCGCAAGCC | |
| | CGGTGCCTGA | |
| 133 | MTEYKPTVRLATRDDVPRAVRTLAAAFADYPATRHTVDPDRHIERVTELQELFLTRVGL | Puromycin |
| | DIGKVWVADDGAAVAVWTTPESVEAGAVFAEIGPRMAELSGSRLAAQQQMEGLLAPHRP | resistance gene |
| | KEPAWFLATVGVSPDHQGKGLGSAVVLPGVEAAERAGVPAFLETSAPRNLPFYERLGFT | (aa) |
| | VTADVEVPEGPRTWCMTRKPGA | |
| 134 | PTQAGEGTLSEALLQLQFDDEDLGALLGNSTDPAVFTDLASVDNSEFQQLLNQGIPVAP | p65AD (aa) |
| | HTTEPMLMEYPEAITRLVTGAQRPPDPAPAPLGAPGLPNGLLSGDEDFSSIADMDFSAL | |
| | LSQISS | |
| 135 | RDSREGMFLPKPEAGSAISDVFEGREVCQPKRIRPFHPPGSPWANRPLPASLAPTPTGP | Rta domain |
| | VHEPVGSLTPAPVPQPLDPAPAVTPEASHLLEDPDEETSQAVKALREMADTVIPQKEEA | (aa) |
| | AICGQMDLSHPPPRGHLDELTTTLESMTEDLNLDSPLTPELNEILDTFLNDECLLHAMH | |
| | ISTGLSIFDTSLF | |
| 136 | GFSVDTSALLDLFSPSVTVPDMSLPDLDSSLASIQELLSPQEPPRPPEAENSSPDSGKQ | HSF1 domain |
| | LVHYTAQPLFLLDPGSVDTGSNDLPVLFELGEGSYFSEGDGFAEDPTISLLTGSEPPKA | (aa) |
| | KDPTVS | |
| 137 | DALDDFDLDMLGSDALDDFDLDMLGSDALDDFDLDMLGSDALDDFDLDMLGSPKKKRKV | VPR (aa) |
| | GSQYLPDTDDRHRIEEKRKRTYETFKSIMKKSPFSGPTDPRPPPRRIAVPSRSSASVPK | |
| | PAPQPYPFTSSLSTINYDEFPTMVFPSGQISQASALAPAPPQVLPQAPAPAPAPAMVSA | |
| | LAQAPAPVPVLAPGPPQAVAPPAPKPTQAGEGTLSEALLQLQFDDEDLGALLGNSTDPA | |
| | VFTDLASVDNSEFQQLLNQGIPVAPHTTEPMLMEYPEAITRLVTGAQRPPDPAPAPLGA | |
| | PGLPNGLLSGDEDESSIADMDFSALLSQISSGSGSGSRDSREGMFLPKPEAGSAISDVE | |
| | EGREVCQPKRIRPFHPPGSPWANRPLPASLAPTPTGPVHEPVGSLTPAPVPQPLDPAPA | |
| | VTPEASHLLEDPDEETSQAVKALREMADTVIPQKEEAAICGQMDLSHPPPRGHLDELTT | |
| | TLESMTEDLNLDSPLTPELNEILDTFLNDECLLHAMHISTGLSIFDTSLF | |
| 138 | DALDDFDLDMLGSDALDDEDLDMLGSDALDDFDLDMLGSDALDDEDLDMLGSLPSASVE | VPH (aa) |
| | FEGSGGPSGQISNQALALAPSSAPVLAQTMVPSSAMVPLAQPPAPAPVLTPGPPQSLSA | |
| | PVPKSTQAGEGTLSEALLHLQFDADEDLGALLGNSTDPGVFTDLASVDNSEFQQLLNQG | |
| | VSMSHSTAEPMLMEYPEAITRLVTGSQRPPDPAPTPLGTSGLPNGLSGDEDESSIADMD | |
| | FSALLSQISSSGQGGGGSGFSVDTSALLDLFSPSVTVPDMSLPDLDSSLASIQELLSPQ | |
| | EPPRPPEAENSSPDSGKQLVHYTAQPLFLLDPGSVDTGSNDLPVLFELGEGSYFSEGDG | |
| | FAEDPTISLLTGSEPPKAKDPTVS | |
| 139 | LPTCSCLDRVIQKDKGPYYTHLGAGPSVAAVREIMENRYGQKGNAIRIEIVVYTGKEGK | TET1 catalytic |
| | SSHGCPIAKWVLRRSSDEEKVLCLVRQRTGHHCPTAVMVVLIMVWDGIPLPMADRLYTE | domain (aa) |
| | LTENLKSYNGHPTDRRCTLNENRTCTCQGIDPETCGASFSFGCSWSMYFNGCKFGRSPS | |
| | PRRFRIDPSSPLHEKNLEDNLQSLATRLAPIYKQYAPVAYQNQVEYENVARECRLGSKE | |
| | GRPFSGVTACLDFCAHPHRDIHNMNNGSTVVCTLTREDNRSLGVIPQDEQLHVLPLYKL | |
| | SDTDEFGSKEGMEAKIKSGAIEVLAPRRKKRTCFTQPVPRSGKKRAAMMTEVLAHKIRA | |
| | VEKKPIPRIKRKNNSTTTNNSKPSSLPTLGSNTETVQPEVKSETEPHFILKSSDNTKTY | |
| | SLMPSAPHPVKEASPGFSWSPKTASATPAPLKNDATASCGFSERSSTPHCTMPSGRLSG | |
| | ANAAAADGPGISQLGEVAPLPTLSAPVMEPLINSEPSTGVTEPLTPHQPNHQPSFLTSP | |
| | QDLASSPMEEDEQHSEADEPPSDEPLSDDPLSPAEEKLPHIDEYWSDSEHIFLDANIGG | |
| | VAIAPAHGSVLIECARRELHATTPVEHPNRNHPTRLSLVFYQHKNLNKPQHGFELNKIK | |
| | FEAKEAKNKKMKASEQKDQAANEGPEQSSEVNELNQIPSHKALTLTHDNVVTVSPYALT | |
| | HVAGPYNHWV | |
| 140 | MLSGKKAAAAAAAAAAAATGTEAGPGTAGGSENGSEVAAQPAGLSGPAEVGPGAVGERT | LSD1 (aa) |
| | PRKKEPPRASPPGGLAEPPGSAGPQAGPTVVPGSATPMETGIAETPEGRRTSRRKRAKV | |
| | EYREMDESLANLSEDEYYSEEERNAKAEKEKKLPPPPPQAPPEEENESEPEEPSGVEGA | |
| | AFQSRLPHDRMTSQEAACFPDIISGPQQTQKVFLFIRNRTLQLWLDNPKIQLTFEATLQ | |
| | QLEAPYNSDTVLVHRVHSYLERHGLINFGIYKRIKPLPTKKTGKVIIIGSGVSGLAAAR | |
| | QLQSFGMDVTLLEARDRVGGRVATFRKGNYVADLGAMVVTGLGGNPMAVVSKQVNMELA | |
| | KIKQKCPLYEANGQAVPKEKDEMVEQEFNRLLEATSYLSHQLDENVLNNKPVSLGQALE | |
| | VVIQLQEKHVKDEQIEHWKKIVKTQEELKELLNKMVNLKEKIKELHQQYKEASEVKPPR | |
| | DITAEFLVKSKHRDLTALCKEYDELAETQGKLEEKLQELEANPPSDVYLSSRDRQILDW | |
| | HFANLEFANATPLSTLSLKHWDQDDDFEFTGSHLTVRNGYSCVPVALAEGLDIKLNTAV | |
| | RQVRYTASGCEVIAVNTRSTSQTFIYKCDAVLCTLPLGVLKQQPPAVQFVPPLPEWKTS | |
| | AVQRMGFGNLNKVVLCFDRVFWDPSVNLFGHVGSTTASRGELFLEWNLYKAPILLALVA | |
| | GEAAGIMENISDDVIVGRCLAILKGIFGSSAVPQPKETVVSRWRADPWARGSYSYVAAG | |
| | SSGNDYDLMAQPITPGPSIPGAPQPIPRLFFAGEHTIRNYPATVHGALLSGLREAGRIA | |
| | DQFLGAMYTLPRQATPGVPAQQSPSM | |
| 141 | NGG, where N is any nucleotide | PAM-SpCas9, |
| | | F. Novicida |
| | | Cas9 |
| 142 | NNGRRT, where N is any nucleotide, and R is G or A | PAM-SaCas9 |
| 143 | NNNNGATT, where N is any nucleotide | PAM-N. |
| | | meningitidis |
| | | Cas9 |
| 144 | NNNNRYAC, where N is any nucleotide, R is G or A, and Y is | PAM-C. |
| | C or T | jejuni Cas9 |
| 145 | NNAGAAW, where N is any nucleotide and W is A or T | PAM-S. |
| | | thermophilus |
| | | Cas9 |
| 146 | NAAAAC, where N is any nucleotide | PAM-T. |
| | | denticola Cas9 |
| 147 | TTTV, where V is A, C, or G | PAM- |
| | | Cas12a/Cpf1Cas9 |
| 148 | NGAN, where N is any nucleotide | Variant PAM- |
| | | SpCas9 variant |
| 149 | NGNG, where N is any nucleotide | Variant PAM- |
| | | SpCas9 variant |
| 150 | NGAG, where N is any nucleotide | Variant PAM- |
| | | SpCas9 variant |
| 151 | NGCG, where N is any nucleotide | Variant PAM- |
| | | SpCas9 variant |
| 152 | LLPKNYHLENEVARLKKLVGER | SunTag GCN4 |
| | | peptide (aa) |
| 153 | GGSGG | GGSGG linker |
| | | (aa) |
| 154 | GGGGG | GGGGG linker |
| | | (aa) |
| 155 | GGAGG | GGAGG linker |
| | | (aa) |
| 156 | GGGGSSS | GGGGSSS |
| | | linker (aa) |
| 157 | GGGGAAA | GGGGAAA |
| | | linker (aa) |
| 158 | GGGGS | (GGGGS)n |
| | | linker (aa), n is |
| | | 1 to 10 |
| 159 | PKKKRKV | SV40 virus |
| | | large T-antigen |
| | | NLS |
| 160 | PAAKRVKLD | c-myc NLS |
| | | (aa) |
| 161 | RQRRNELKRSP | c-myc NLS |
| | | (aa) |
| 162 | NOSSNFGPMKGGNFGGRSSGPYGGGGQYFAKPRNQGGY | hRNPA1 M9 |
| | | NLS (aa) |
| 163 | RMRIZFKNKGKDTAELRRRRVEVSVELRKAKKDEQILKRRNV | importin-alpha |
| | | IBB domain |
| | | NLS (aa) |
| 164 | VSRKRPRP | myoma T |
| | | protein NLS |
| | | (aa) |
| 165 | PPKKARED | myoma T |
| | | protein NLS |
| | | (aa) |
| 166 | PQPKKKPL | human p53 |
| | | NLS (aa) |
| 167 | SALIKKKKKMAP | mouse c-abl IV |
| | | NLS (aa) |
| 168 | DRLRR | influenza virus |
| | | NS1 NLS (aa) |
| 169 | PKQKKRK | influenza virus |
| | | NS1 NLS (aa) |
| 170 | RKLKKKIKKL | Hepatitis virus |
| | | delta antigen |
| | | NLS (aa) |
| 171 | REKKKFLKRR | mouse Mx1 |
| | | protein MNLS |
| | | (aa) |
| 172 | KRKGDEVDGVDEVAKKKSKK | human |
| | | poly(ADP- |
| | | ribose) |
| | | polymerase |
| | | NLS (aa) |
| 173 | RKCLQAGMNLEARKTKK | steroid |
| | | hormone |
| | | receptors |
| | | (human) |
| | | glucocorticoid |
| | | NLS (aa) |
| 174 | SGSETPGTSESATPES | Linker (aa) |
| 175 | GACAAGAAGTACTCCATTGGGCTCGCCATCGGCACAAACAGCGTCGGCTGGGCCGTCAT | N term |
| | TACGGACGAGTACAAGGTGCCGAGCAAAAAATTCAAAGTTCTGGGCAATACCGATCGCC | dSpCas9 N |
| | ACAGCATAAAGAAGAACCTCATTGGCGCCCTCCTGTTCGACTCCGGGGAAACCGCCGAA | terminal |
| | GCCACGCGGCTCAAAAGAACAGCACGGCGCAGATATACCCGCAGAAAGAATCGGATCTG | fragement (nt) |
| | CTACCtgcaGGAGATCTTTAGTAATGAGATGGCTAAGGTGGATGACTCTTTCTTCCATA | |
| | GGCTGGAGGAGTCCTTTTTGGTGGAGGAGGATAAAAAGCACGAGCGCCACCCAATCTTT | |
| | GGCAATATCGTGGACGAGGTGGCGTACCATGAAAAGTACCCAACCATATATCATCTGAG | |
| | GAAGAAGCTTGTAGACAGTACTGATAAGGCTGACTTGCGGTTGATCTATCTCGCGCTGG | |
| | CGCATATGATCAAATTTCGGGGACACTTCCTCATCGAGGGGGACCTGAACCCAGACAAC | |
| | AGCGATGTCGACAAACTCTTTATCCAACTGGTTCAGACTTACAATCAGCTTTTCGAAGA | |
| | GAACCCGATCAACGCATCCGGAGTTGACGCCAAAGCAATCCTGAGCGCTAGGCTGTCCA | |
| | AATCCCGGCGGCTCGAAAACCTCATCGCACAGCTCCCTGGGGAGAAGAAGAACGGCCTG | |
| | TTTGGTAATCTTATCGCCCTGTCACTCGGGCTGACCCCCAACTTTAAATCTAACTTCGA | |
| | CCTGGCCGAAGATGCCAAGCTTCAACTGAGCAAAGACACCTACGATGATGATCTCGACA | |
| | ATCTGCTGGCCCAGATCGGCGACCAGTACGCAGACCTTTTTTTGGCGGCAAAGAACCTG | |
| | TCAGACGCCATTCTGCTGAGTGATATTCTGCGAGTGAACACGGAGATCACCAAAGCTCC | |
| | GCTGAGCGCTAGTATGATCAAGCGCTATGATGAGCACCACCAAGACTTGACTTTGCTGA | |
| | AGGCCCTTGTCAGACAGCAACTGCCTGAGAAGTACAAGGAAATTTTCTTCGATCAGTCT | |
| | AAAAATGGCTACGCCGGATACATTGACGGCGGAGCAAGCCAGGAGGAATTTTACAAATT | |
| | TATTAAGCCCATCTTGGAAAAAATGGACGGCACCGAGGAGCTGCTGGTAAAGCTTAACA | |
| | GAGAAGATCTGTTGCGCAAACAGCGCACTTTCGACAATGGAAGCATCCCCCACCAGATT | |
| | CACCTGGGCGAACTGCACGCTATCCTCAGGCGGCAAGAGGATTTCTACCCCTTTTTGAA | |
| | AGATAACAGGGAAAAGATTGAGAAAATCCTCACATTTCGGATACCCTACTATGTAGGCC | |
| | CCCTCGCCCGGGGAAATTCCAGATTCGCGTGGATGACTCGCAAATCAGAAGAGACCATC | |
| | ACTCCCTGGAACTTCGAGGAAGTCGTGGATAAGGGGGCCTCTGCCCAGTCCTTCATCGA | |
| | AAGGATGACTAACTTTGATAAAAATCTGCCTAACGAAAAGGTGCTTCCTAAACACTCTC | |
| | TGCTGTACGAGTACTTCACAGTTTATAACGAGCTCACCAAGGTCAAATACGTCACAGAA | |
| | GGGATGAGAAAGCCAGCATTCCTGTCTGGAGAGCAGAAGAAAGCTATCGTGGACCTCCT | |
| | CTTCAAGACGAACCGGAAAGTTACCGTGAAACAGCTCAAAGAAGACTATTTCAAAAAGA | |
| | TTGAA | |
| 176 | DKKYSIGLAIGTNSVGWAVITDEYKVPSKKFKVLGNTDRHSIKKNLIGALLFDSGETAE | N term |
| | ATRLKRTARRRYTRRKNRICYLQEIFSNEMAKVDDSFFHRLEESFLVEEDKKHERHPIF | dSpCas9 N |
| | GNIVDEVAYHEKYPTIYHLRKKLVDSTDKADLRLIYLALAHMIKFRGHFLIEGDLNPDN | terminal |
| | SDVDKLFIQLVQTYNQLFEENPINASGVDAKAILSARLSKSRRLENLIAQLPGEKKNGL | fragement |
| | FGNLIALSLGLTPNFKSNFDLAEDAKLQLSKDTYDDDLDNLLAQIGDQYADLFLAAKNL | |
| | SDAILLSDILRVNTEITKAPLSASMIKRYDEHHQDLTLLKALVRQQLPEKYKEIFFDQS | |
| | KNGYAGYIDGGASQEEFYKFIKPILEKMDGTEELLVKLNREDLLRKQRTEDNGSIPHQI | |
| | HLGELHAILRRQEDFYPFLKDNREKIEKILTFRIPYYVGPLARGNSRFAWMTRKSEETI | |
| | TPWNFEEVVDKGASAQSFIERMTNFDKNLPNEKVLPKHSLLYEYFTVYNELTKVKYVTE | |
| | GMRKPAFLSGEQKKAIVDLLFKTNRKVTVKQLKEDYFKKIE | |
| 177 | TGCCTGTCCTACGAGACAGAGATCCTGACAGTGGAGTATGGCCTGCTGCCAATCGGCAA | N term Npu |
| | GATCGTGGAGAAGAGGATCGAGTGTACCGTGTACTCTGTGGATAACAATGGCAACATCT | Intein (nt) |
| | ATACACAGCCCGTGGCACAGTGGCACGATAGGGGAGAGCAGGAGGTGTTCGAGTATTGC | |
| | CTGGAGGACGGCAGCCTGATCAGGGCAACCAAGGACCACAAGTTCATGACAGTGGATGG | |
| | CCAGATGCTGCCCATCGAC | |
| 178 | CLSYETEILTVEYGLLPIGKIVEKRIECTVYSVDNNGNIYTQPVAQWHDRGEQEVFEYC | N term Npu |
| | LEDGSLIRATKDHKFMTVDGQMLPID | Intein (aa) |
| 179 | ATGAAACGGACAGCCGACGGAAGCGAGTTCGAGTCACCAAAGAAGAAGCGGAAAGTCAT | dSpCas9-573- |
| | CAAGATTGCTACACGGAAATACCTGGGAAAGCAGAACGTGTACGACATCGGCGTGGAGC | C Intein (nt) |
| | GGGATCACAACTTCGCCCTGAAGAATGGCTTTATCGCCAGCAATTGTTTCGACTCTGTT | |
| | GAAATCAGCGGAGTGGAGGATCGCTTCAACGCATCCCTGGGAACGTATCACGATCTCCT | |
| | GAAAATCATTAAAGACAAGGACTTCCTGGACAATGAGGAGAACGAGGACATTCTTGAGG | |
| | ACATTGTCCTCACCCTTACGTTGTTTGAAGATAGGGAGATGATTGAAGAACGCTTGAAA | |
| | ACTTACGCTCATCTCTTCGACGACAAAGTCATGAAACAGCTCAAGAGGCGCCGATATAC | |
| | AGGATGGGGGCGGCTGTCAAGAAAACTGATCAATGGgatcCGAGACAAGCAGAGTGGAA | |
| | AGACAATCCTGGATTTTCTTAAGTCCGATGGATTTGCCAACCGGAACTTCATGCAGTTG | |
| | ATCCATGATGACTCTCTCACCTTTAAGGAGGACATCCAGAAAGCACAAGTTTCTGGCCA | |
| | GGGGGACAGTCTTCACGAGCACATCGCTAATCTTGCAGGTAGCCCAGCTATCAAAAAGG | |
| | GAATACTGCAGACCGTTAAGGTCGTGGATGAACTCGTCAAAGTAATGGGAAGGCATAAG | |
| | CCCGAGAATATCGTTATCGAGATGGCCCGAGAGAACCAAACTACCCAGAAGGGACAGAA | |
| | GAACAGTAGGGAAAGGATGAAGAGGATTGAAGAGGGTATAAAAGAACTGGGGTCCCAAA | |
| | TCCTTAAGGAACACCCAGTTGAAAACACCCAGCTTCAGAATGAGAAGCTCTACCTGTAC | |
| | TACCTGCAGAACGGCAGGGACATGTACGTGGATCAGGAACTGGACATCAATCGGCTCTC | |
| | CGACTACGACGTGGATGCCATCGTGCCCCAGTCTTTTCTCAAAGATGATTCTATTGATA | |
| | ATAAAGTGTTGACAAGATCCGATAAAAATAGAGGGAAGAGTGATAACGTCCCCTCAGAA | |
| | GAAGTTGTCAAGAAAATGAAAAATTATTGGCGGCAGCTGCTGAACGCCAAACTGATCAC | |
| | ACAACGGAAGTTCGATAATCTGACTAAGGCTGAACGAGGTGGCCTGTCTGAGTTGGATA | |
| | AAGCCGGCTTCATCAAAAGGCAGCTTGTTGAGACACGCCAGATCACCAAgcacGTGGCC | |
| | CAAATTCTCGATTCACGCATGAACACCAAGTACGATGAAAATGACAAACTGATTCGAGA | |
| | GGTGAAAGTTATTACTCTGAAGTCTAAGCTGGTCTCAGATTTCAGAAAGGACTTTCAGT | |
| | TTTATAAGGTGAGAGAGATCAACAATTACCACCATGCGCATGATGCCTACCTGAATGCA | |
| | GTGGTAGGCACTGCACTTATCAAAAAATATCCCAAGCTTGAATCTGAATTTGTTTACGG | |
| | AGACTATAAAGTGTACGATGTTAGGAAAATGATCGCAAAGTCTGAGCAGGAAATAGGCA | |
| | AGGCCACCGCTAAGTACTTCTTTTACAGCAATATTATGAATTTTTTCAAGACCGAGATT | |
| | ACACTGGCCAATGGAGAGATTCGGAAGCGACCACTTATCGAAACAAACGGAGAAACAGG | |
| | AGAAATCGTGTGGGACAAGGGTAGGGATTTCGCGACAGTCCGGAAGGTCCTGTCCATGC | |
| | CGCAGGTGAACATCGTTAAAAAGACCGAAGTACAGACCGGAGGCTTCTCCAAGGAAAGT | |
| | ATCCTCCCGAAAAGGAACAGCGACAAGCTGATCGCACGCAAAAAAGATTGGGACCCCAA | |
| | GAAATACGGCGGATTCGATTCTCCTACAGTCGCTTACAGTGTACTGGTTGTGGCCAAAG | |
| | TGGAGAAAGGGAAGTCTAAAAAACTCAAAAGCGTCAAGGAACTGCTGGGCATCACAATC | |
| | ATGGAGCGATCAAGCTTCGAAAAAAACCCCATCGACTTTCTCGAGGCGAAAGGATATAA | |
| | AGAGGTCAAAAAAGACCTCATCATTAAGCTTCCCAAGTACTCTCTCTTTGAGCTTGAAA | |
| | ACGGCCGGAAACGAATGCTCGCTAGTGCGGGCGAGCTGCAGAAAGGTAACGAGCTGGCA | |
| | CTGCCCTCTAAATACGTTAATTTCTTGTATCTGGCCAGCCACTATGAAAAGCTCAAAGG | |
| | GTCTCCCGAAGATAATGAGCAGAAGCAGCTGTTCGTGGAACAACACAAACACTACCTTG | |
| | ATGAGATCATCGAGCAAATAAGCGAATTCTCCAAAAGAGTGATCCTCGCCGACGCTAAC | |
| | CTCGATAAGGTGCTTTCTGCTTACAATAAGCACAGGGATAAGCCCATCAGGGAGCAGGC | |
| | AGAAAACATTATCCACTTGTTTACTCTGACCAACTTGGGCGCGCCTGCAGCCTTCAAGT | |
| | ACTTCGACACCACCATAGACAGAAAGCGGTACACCTCTACAAAGGAGGTCCTGGACGCC | |
| | ACACTGATTCATCAGTCAATTACGGGGCTCTATGAAACAAGAATCGACCTCTCTCAGCT | |
| | CGGTGGAGACAAAAGGCCGGCGGCCACGAAAAAGGCCGGCCAGGCAAAAAAGAAAAAGT | |
| | GA | |
| 180 | MKRTADGSEFESPKKKRKVIKIATRKYLGKQNVYDIGVERDHNFALKNGFIASNCFDSV | dSpCas9-573- |
| | EISGVEDRFNASLGTYHDLLKIIKDKDFLDNEENEDILEDIVLTLTLFEDREMIEERLK | C Intein (aa) |
| | TYAHLFDDKVMKQLKRRRYTGWGRLSRKLINGIRDKQSGKTILDFLKSDGFANRNFMQL | |
| | IHDDSLTFKEDIQKAQVSGQGDSLHEHIANLAGSPAIKKGILQTVKVVDELVKVMGRHK | |
| | PENIVIEMARENQTTQKGQKNSRERMKRIEEGIKELGSQILKEHPVENTQLQNEKLYLY | |
| | YLQNGRDMYVDQELDINRLSDYDVDAIVPQSFLKDDSIDNKVLTRSDKNRGKSDNVPSE | |
| | EVVKKMKNYWRQLLNAKLITQRKFDNLTKAERGGLSELDKAGFIKRQLVETRQITKHVA | |
| | QILDSRMNTKYDENDKLIREVKVITLKSKLVSDFRKDFQFYKVREINNYHHAHDAYLNA | |
| | VVGTALIKKYPKLESEFVYGDYKVYDVRKMIAKSEQEIGKATAKYFFYSNIMNFFKTEI | |
| | TLANGEIRKRPLIETNGETGEIVWDKGRDFATVRKVLSMPQVNIVKKTEVQTGGFSKES | |
| | ILPKRNSDKLIARKKDWDPKKYGGFDSPTVAYSVLVVAKVEKGKSKKLKSVKELLGITI | |
| | MERSSFEKNPIDFLEAKGYKEVKKDLIIKLPKYSLFELENGRKRMLASAGELQKGNELA | |
| | LPSKYVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKEVLDA | |
| | TLIHQSITGLYETRIDLSQLGGDKRPAATKKAGQAKKKK | |
| 181 | ATCAAGATTGCTACACGGAAATACCTGGGAAAGCAGAACGTGTACGACATCGGCGTGGA | C term Npu |
| | GCGGGATCACAACTTCGCCCTGAAGAATGGCTTTATCGCCAGCAAT | Intein (nt) |
| 182 | IKIATRKYLGKQNVYDIGVERDHNFALKNGFIASN | C term Npu |
| | | Intein (aa) |
| 183 | TGTTTCGACTCTGTTGAAATCAGCGGAGTGGAGGATCGCTTCAACGCATCCCTGGGAAC | C term |
| | GTATCACGATCTCCTGAAAATCATTAAAGACAAGGACTTCCTGGACAATGAGGAGAACG | dSpCas9 C |
| | AGGACATTCTTGAGGACATTGTCCTCACCCTTACGTTGTTTGAAGATAGGGAGATGATT | terminal |
| | GAAGAACGCTTGAAAACTTACGCTCATCTCTTCGACGACAAAGTCATGAAACAGCTCAA | fragment (nt) |
| | GAGGCGCCGATATACAGGATGGGGGCGGCTGTCAAGAAAACTGATCAATGGgatcCGAG | |
| | ACAAGCAGAGTGGAAAGACAATCCTGGATTTTCTTAAGTCCGATGGATTTGCCAACCGG | |
| | AACTTCATGCAGTTGATCCATGATGACTCTCTCACCTTTAAGGAGGACATCCAGAAAGC | |
| | ACAAGTTTCTGGCCAGGGGGACAGTCTTCACGAGCACATCGCTAATCTTGCAGGTAGCC | |
| | CAGCTATCAAAAAGGGAATACTGCAGACCGTTAAGGTCGTGGATGAACTCGTCAAAGTA | |
| | ATGGGAAGGCATAAGCCCGAGAATATCGTTATCGAGATGGCCCGAGAGAACCAAACTAC | |
| | CCAGAAGGGACAGAAGAACAGTAGGGAAAGGATGAAGAGGATTGAAGAGGGTATAAAAG | |
| | AACTGGGGTCCCAAATCCTTAAGGAACACCCAGTTGAAAACACCCAGCTTCAGAATGAG | |
| | AAGCTCTACCTGTACTACCTGCAGAACGGCAGGGACATGTACGTGGATCAGGAACTGGA | |
| | CATCAATCGGCTCTCCGACTACGACGTGGATGCCATCGTGCCCCAGTCTTTTCTCAAAG | |
| | ATGATTCTATTGATAATAAAGTGTTGACAAGATCCGATAAAAATAGAGGGAAGAGTGAT | |
| | AACGTCCCCTCAGAAGAAGTTGTCAAGAAAATGAAAAATTATTGGCGGCAGCTGCTGAA | |
| | CGCCAAACTGATCACACAACGGAAGTTCGATAATCTGACTAAGGCTGAACGAGGTGGCC | |
| | TGTCTGAGTTGGATAAAGCCGGCTTCATCAAAAGGCAGCTTGTTGAGACACGCCAGATC | |
| | ACCAAgcacGTGGCCCAAATTCTCGATTCACGCATGAACACCAAGTACGATGAAAATGA | |
| | CAAACTGATTCGAGAGGTGAAAGTTATTACTCTGAAGTCTAAGCTGGTCTCAGATTTCA | |
| | GAAAGGACTTTCAGTTTTATAAGGTGAGAGAGATCAACAATTACCACCATGCGCATGAT | |
| | GCCTACCTGAATGCAGTGGTAGGCACTGCACTTATCAAAAAATATCCCAAGCTTGAATC | |
| | TGAATTTGTTTACGGAGACTATAAAGTGTACGATGTTAGGAAAATGATCGCAAAGTCTG | |
| | AGCAGGAAATAGGCAAGGCCACCGCTAAGTACTTCTTTTACAGCAATATTATGAATTTT | |
| | TTCAAGACCGAGATTACACTGGCCAATGGAGAGATTCGGAAGCGACCACTTATCGAAAC | |
| | AAACGGAGAAACAGGAGAAATCGTGTGGGACAAGGGTAGGGATTTCGCGACAGTCCGGA | |
| | AGGTCCTGTCCATGCCGCAGGTGAACATCGTTAAAAAGACCGAAGTACAGACCGGAGGC | |
| | TTCTCCAAGGAAAGTATCCTCCCGAAAAGGAACAGCGACAAGCTGATCGCACGCAAAAA | |
| | AGATTGGGACCCCAAGAAATACGGCGGATTCGATTCTCCTACAGTCGCTTACAGTGTAC | |
| | TGGTTGTGGCCAAAGTGGAGAAAGGGAAGTCTAAAAAACTCAAAAGCGTCAAGGAACTG | |
| | CTGGGCATCACAATCATGGAGCGATCAAGCTTCGAAAAAAACCCCATCGACTTTCTCGA | |
| | GGCGAAAGGATATAAAGAGGTCAAAAAAGACCTCATCATTAAGCTTCCCAAGTACTCTC | |
| | TCTTTGAGCTTGAAAACGGCCGGAAACGAATGCTCGCTAGTGCGGGCGAGCTGCAGAAA | |
| | GGTAACGAGCTGGCACTGCCCTCTAAATACGTTAATTTCTTGTATCTGGCCAGCCACTA | |
| | TGAAAAGCTCAAAGGGTCTCCCGAAGATAATGAGCAGAAGCAGCTGTTCGTGGAACAAC | |
| | ACAAACACTACCTTGATGAGATCATCGAGCAAATAAGCGAATTCTCCAAAAGAGTGATC | |
| | CTCGCCGACGCTAACCTCGATAAGGTGCTTTCTGCTTACAATAAGCACAGGGATAAGCC | |
| | CATCAGGGAGCAGGCAGAAAACATTATCCACTTGTTTACTCTGACCAACTTGGGCGCGC | |
| | CTGCAGCCTTCAAGTACTTCGACACCACCATAGACAGAAAGCGGTACACCTCTACAAAG | |
| | GAGGTCCTGGACGCCACACTGATTCATCAGTCAATTACGGGGCTCTATGAAACAAGAAT | |
| | CGACCTCTCTCAGCTCGGTGGAGAC | |
| 184 | CFDSVEISGVEDRFNASLGTYHDLLKIIKDKDFLDNEENEDILEDIVLTLTLFEDREMI | C term |
| | EERLKTYAHLFDDKVMKQLKRRRYTGWGRLSRKLINGIRDKQSGKTILDFLKSDGFANR | dSpCas9 C |
| | NFMQLIHDDSLTFKEDIQKAQVSGQGDSLHEHIANLAGSPAIKKGILQTVKVVDELVKV | terminal |
| | MGRHKPENIVIEMARENQTTQKGQKNSRERMKRIEEGIKELGSQILKEHPVENTQLQNE | fragment (aa) |
| | KLYLYYLQNGRDMYVDQELDINRLSDYDVDAIVPQSFLKDDSIDNKVLTRSDKNRGKSD | |
| | NVPSEEVVKKMKNYWRQLLNAKLITQRKEDNLTKAERGGLSELDKAGFIKRQLVETRQI | |
| | TKHVAQILDSRMNTKYDENDKLIREVKVITLKSKLVSDFRKDFQFYKVREINNYHHAHD | |
| | AYLNAVVGTALIKKYPKLESEFVYGDYKVYDVRKMIAKSEQEIGKATAKYFFYSNIMNE | |
| | FKTEITLANGEIRKRPLIETNGETGEIVWDKGRDFATVRKVLSMPQVNIVKKTEVQTGG | |
| | FSKESILPKRNSDKLIARKKDWDPKKYGGFDSPTVAYSVLVVAKVEKGKSKKLKSVKEL | |
| | LGITIMERSSFEKNPIDFLEAKGYKEVKKDLIIKLPKYSLFELENGRKRMLASAGELQK | |
| | GNELALPSKYVNFLYLASHYEKLKGSPEDNEQKQLFVEQHKHYLDEIIEQISEFSKRVI | |
| | LADANLDKVLSAYNKHRDKPIREQAENIIHLFTLTNLGAPAAFKYFDTTIDRKRYTSTK | |
| | EVLDATLIHQSITGLYETRIDLSQLGGD | |
| 185 | GGAGGGCCGAGCTCTGGCGCACCCCCACCAAGTGGAGGGTCTCCTGCCGGGTCCCCAAC | extended linker |
| | ATCTACTGAAGAAGGCACCAGCGAATCCGCAACGCCCGAGTCAGGCCCTGGTACCTCCA | (nt) |
| | CAGAACCATCTGAAGGTAGTGCGCCTGGTTCCCCAGCTGGAAGCCCTACTTCCACCGAA | |
| | GAAGGCACGTCAACCGAACCAAGTGAAGGATCTGCCCCTGGGACCAGCACTGAACCATC | |
| | TGAG | |
| 186 | GGPSSGAPPPSGGSPAGSPTSTEEGTSESATPESGPGTSTEPSEGSAPGSPAGSPTSTE | extended linker |
| | EGTSTEPSEGSAPGTSTEPSE | (aa) |
| 187 | AACCCAAAGAAGAAGCGGAAGGTCGGTATCCACGGAGTCCCAGCAGCC | linker (nt) |
| 188 | NPKKKRKVGIHGVPAA | linker (aa) |
| 189 | MKRNYILGLAIGITSVGYGIIDYETRDVIDAGVRLFKEANVENNEGRRSKRGARRLKRR | dSaCas9 (aa) |
| | RRHRIQRVKKLLFDYNLLTDHSELSGINPYEARVKGLSQKLSEEEFSAALLHLAKRRGV | |
| | HNVNEVEEDTGNELSTKEQISRNSKALEEKYVAELQLERLKKDGEVRGSINRFKTSDYV | |
| | KEAKQLLKVQKAYHQLDQSFIDTYIDLLETRRTYYEGPGEGSPFGWKDIKEWYEMLMGH | |
| | CTYFPEELRSVKYAYNADLYNALNDLNNLVITRDENEKLEYYEKFQIIENVFKQKKKPT | |
| | LKQIAKEILVNEEDIKGYRVTSTGKPEFTNLKVYHDIKDITARKEIIENAELLDQIAKI | |
| | LTIYQSSEDIQEELTNLNSELTQEEIEQISNLKGYTGTHNLSLKAINLILDELWHINDN | |
| | QIAIFNRLKLVPKKVDLSQQKEIPTTLVDDFILSPVVKRSFIQSIKVINAIIKKYGLPN | |
| | DIIIELAREKNSKDAQKMINEMQKRNRQTNERIEEIIRTTGKENAKYLIEKIKLHDMQE | |
| | GKCLYSLEAIPLEDLLNNPFNYEVDHIIPRSVSFDNSFNNKVLVKQEEASKKGNRTPFQ | |
| | YLSSSDSKISYETFKKHILNLAKGKGRISKTKKEYLLEERDINRFSVQKDFINRNLVDT | |
| | RYATRGLMNLLRSYFRVNNLDVKVKSINGGFTSFLRRKWKFKKERNKGYKHHAEDALII | |
| | ANADFIFKEWKKLDKAKKVMENQMFEEKQAESMPEIETEQEYKEIFITPHQIKHIKDFK | |
| | DYKYSHRVDKKPNRELINDTLYSTRKDDKGNTLIVNNLNGLYDKDNDKLKKLINKSPEK | |
| | LLMYHHDPQTYQKLKLIMEQYGDEKNPLYKYYEETGNYLTKYSKKDNGPVIKKIKYYGN | |
| | KLNAHLDITDDYPNSRNKVVKLSLKPYRFDVYLDNGVYKFVTVKNLDVIKKENYYEVNS | |
| | KCYEEAKKLKKISNQAEFIASFYNNDLIKINGELYRVIGVNNDLLNRIEVNMIDITYRE | |
| | YLENMNDKRPPRIIKTIASKTQSIKKYSTDILGNLYEVKSKKHPQIIKKG | |
| 190 | MDKKYSIGLAIGTNSVGWAVITDEYKVPSKKFKVLGNTDRHSIKKNLIGALLFDSGETA | dSpCas9 (aa) |
| | EATRLKRTARRRYTRRKNRICYLQEIFSNEMAKVDDSFFHRLEESELVEEDKKHERHPI | |
| | FGNIVDEVAYHEKYPTIYHLRKKLVDSTDKADLRLIYLALAHMIKFRGHFLIEGDLNPD | |
| | NSDVDKLFIQLVQTYNQLFEENPINASGVDAKAILSARLSKSRRLENLIAQLPGEKKNG | |
| | LFGNLIALSLGLTPNFKSNFDLAEDAKLQLSKDTYDDDLDNLLAQIGDQYADLFLAAKN | |
| | LSDAILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRENAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDAIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| 191 | MTQFEGFTNLYQVSKTLRFELIPQGKTLKHIQEQGFIEEDKARNDHYKELKPIIDRIYK | dAsCas12a |
| | TYADQCLQLVQLDWENLSAAIDSYRKEKTEETRNALIEEQATYRNAIHDYFIGRTDNLT | (dAsCpf1) (aa) |
| | DAINKRHAEIYKGLFKAELFNGKVLKQLGTVTTTEHENALLRSFDKFTTYFSGFYENRK | |
| | NVFSAEDISTAIPHRIVQDNFPKFKENCHIFTRLITAVPSLREHFENVKKAIGIFVSTS | |
| | IEEVFSFPFYNQLLTQTQIDLYNQLLGGISREAGTEKIKGLNEVLNLAIQKNDETAHII | |
| | ASLPHRFIPLFKQILSDRNTLSFILEEFKSDEEVIQSFCKYKTLLRNENVLETAEALEN | |
| | ELNSIDLTHIFISHKKLETISSALCDHWDTLRNALYERRISELTGKITKSAKEKVQRSL | |
| | KHEDINLQEIISAAGKELSEAFKQKTSEILSHAHAALDQPLPTTLKKQEEKEILKSQLD | |
| | SLLGLYHLLDWFAVDESNEVDPEFSARLTGIKLEMEPSLSFYNKARNYATKKPYSVEKF | |
| | KLNFQMPTLASGWDVNKEKNNGAILFVKNGLYYLGIMPKQKGRYKALSFEPTEKTSEGF | |
| | DKMYYDYFPDAAKMIPKCSTQLKAVTAHFQTHTTPILLSNNFIEPLEITKEIYDLNNPE | |
| | KEPKKFQTAYAKKTGDQKGYREALCKWIDFTRDELSKYTKTTSIDLSSLRPSSQYKDLG | |
| | EYYAELNPLLYHISFQRIAEKEIMDAVETGKLYLFQIYNKDFAKGHHGKPNLHTLYWTG | |
| | LFSPENLAKTSIKLNGQAELFYRPKSRMKRMAHRLGEKMLNKKLKDQKTPIPDTLYQEL | |
| | YDYVNHRLSHDLSDEARALLPNVITKEVSHEIIKDRRFTSDKFFFHVPITLNYQAANSP | |
| | SKFNQRVNAYLKEHPETPIIGIARGERNLIYITVIDSTGKILEQRSLNTIQQFDYQKKL | |
| | DNREKERVAARQAWSVVGTIKDLKQGYLSQVIHEIVDLMIHYQAVVVLANLNFGFKSKR | |
| | TGIAEKAVYQQFEKMLIDKLNCLVLKDYPAEKVGGVLNPYQLTDQFTSFAKMGTQSGFL | |
| | FYVPAPYTSKIDPLIGFVDPFVWKTIKNHESRKHFLEGFDFLHYDVKTGDFILHFKMNR | |
| | NLSFQRGLPGFMPAWDIVFEKNETQFDAKGTPFIAGKRIVPVIENHRFTGRYRDLYPAN | |
| | ELIALLEEKGIVERDGSNILPKLLENDDSHAIDTMVALIRSVLQMRNSNAATGEDYINS | |
| | PVRDLNGVCFDSRFQNPEWPMDADANGAYHIALKGQLLLNHLKESKDLKLQNGISNQDW | |
| | LAYIQELRN | |
| 192 | TQFEGFTNLYQVSKTLRFELIPQGKTLKHIQEQGFIEEDKARNDHYKELKPIIDRIYKT | dAsCas12a |
| | YADQCLQLVQLDWENLSAAIDSYRKEKTEETRNALIEEQATYRNAIHDYFIGRTDNLTD | (dAsCpf1) (aa) |
| | AINKRHAEIYKGLFKAELFNGKVLKQLGTVTTTEHENALLRSFDKFTTYFSGFYENRKN | |
| | VFSAEDISTAIPHRIVQDNFPKFKENCHIFTRLITAVPSLREHFENVKKAIGIFVSTSI | |
| | EEVFSFPFYNQLLTQTQIDLYNQLLGGISREAGTEKIKGLNEVLNLAIQKNDETAHIIA | |
| | SLPHRFIPLFKQILSDRNTLSFILEEFKSDEEVIQSFCKYKTLLRNENVLETAEALENE | |
| | LNSIDLTHIFISHKKLETISSALCDHWDTLRNALYERRISELTGKITKSAKEKVQRSLK | |
| | HEDINLQEIISAAGKELSEAFKQKTSEILSHAHAALDQPLPTTLKKQEEKEILKSQLDS | |
| | LLGLYHLLDWFAVDESNEVDPEFSARLTGIKLEMEPSLSFYNKARNYATKKPYSVEKFK | |
| | LNFQMPTLASGWDVNKEKNNGAILFVKNGLYYLGIMPKQKGRYKALSFEPTEKTSEGED | |
| | KMYYDYFPDAAKMIPKCSTQLKAVTAHFQTHTTPILLSNNFIEPLEITKEIYDLNNPEK | |
| | EPKKFQTAYAKKTGDQKGYREALCKWIDFTRDFLSKYTKTTSIDLSSLRPSSQYKDLGE | |
| | YYAELNPLLYHISFQRIAEKEIMDAVETGKLYLFQIYNKDFAKGHHGKPNLHTLYWTGL | |
| | FSPENLAKTSIKLNGQAELFYRPKSRMKRMAHRLGEKMLNKKLKDQKTPIPDTLYQELY | |
| | DYVNHRLSHDLSDEARALLPNVITKEVSHEIIKDRRFTSDKFFFHVPITLNYQAANSPS | |
| | KFNQRVNAYLKEHPETPIIGIARGERNLIYITVIDSTGKILEQRSLNTIQQFDYQKKLD | |
| | NREKERVAARQAWSVVGTIKDLKQGYLSQVIHEIVDLMIHYQAVVVLANLNFGFKSKRT | |
| | GIAEKAVYQQFEKMLIDKLNCLVLKDYPAEKVGGVLNPYQLTDQFTSFAKMGTQSGFLF | |
| | YVPAPYTSKIDPLTGFVDPFVWKTIKNHESRKHFLEGFDFLHYDVKTGDFILHFKMNRN | |
| | LSFQRGLPGFMPAWDIVFEKNETQFDAKGTPFIAGKRIVPVIENHRFTGRYRDLYPANE | |
| | LIALLEEKGIVERDGSNILPKLLENDDSHAIDTMVALIRSVLQMRNSNAATGEDYINSP | |
| | VRDLNGVCFDSRFQNPEWPMDADANGAYHIALKGQLLLNHLKESKDLKLQNGISNQDWL | |
| | AYIQELRN | |
| 193 | MTQFEGFTNLYQVSKTLRFELIPQGKTLKHIQEQGFIEEDKARNDHYKELKPIIDRIYK | WT AsCas12a |
| | TYADQCLQLVQLDWENLSAAIDSYRKEKTEETRNALIEEQATYRNAIHDYFIGRTDNLT | (AsCpf1) (aa) |
| | DAINKRHAEIYKGLFKAELFNGKVLKQLGTVTTTEHENALLRSFDKFTTYFSGFYENRK | |
| | NVFSAEDISTAIPHRIVQDNFPKFKENCHIFTRLITAVPSLREHFENVKKAIGIFVSTS | |
| | IEEVFSFPFYNQLLTQTQIDLYNQLLGGISREAGTEKIKGLNEVLNLAIQKNDETAHII | |
| | ASLPHRFIPLFKQILSDRNTLSFILEEFKSDEEVIQSFCKYKTLLRNENVLETAEALEN | |
| | ELNSIDLTHIFISHKKLETISSALCDHWDTLRNALYERRISELTGKITKSAKEKVQRSL | |
| | KHEDINLQEIISAAGKELSEAFKQKTSEILSHAHAALDQPLPTTLKKQEEKEILKSQLD | |
| | SLLGLYHLLDWFAVDESNEVDPEFSARLTGIKLEMEPSLSFYNKARNYATKKPYSVEKF | |
| | KLNFQMPTLASGWDVNKEKNNGAILFVKNGLYYLGIMPKQKGRYKALSFEPTEKTSEGF | |
| | DKMYYDYFPDAAKMIPKCSTQLKAVTAHFQTHTTPILLSNNFIEPLEITKEIYDLNNPE | |
| | KEPKKFQTAYAKKTGDQKGYREALCKWIDFTRDFLSKYTKTTSIDLSSLRPSSQYKDLG | |
| | EYYAELNPLLYHISFQRIAEKEIMDAVETGKLYLFQIYNKDFAKGHHGKPNLHTLYWTG | |
| | LFSPENLAKTSIKLNGQAELFYRPKSRMKRMAHRLGEKMLNKKLKDQKTPIPDTLYQEL | |
| | YDYVNHRLSHDLSDEARALLPNVITKEVSHEIIKDRRFTSDKFFFHVPITLNYQAANSP | |
| | SKFNQRVNAYLKEHPETPIIGIDRGERNLIYITVIDSTGKILEQRSLNTIQQFDYQKKL | |
| | DNREKERVAARQAWSVVGTIKDLKQGYLSQVIHEIVDLMIHYQAVVVLENLNFGFKSKR | |
| | TGIAEKAVYQQFEKMLIDKLNCLVLKDYPAEKVGGVLNPYQLTDQFTSFAKMGTQSGEL | |
| | FYVPAPYTSKIDPLTGFVDPFVWKTIKNHESRKHFLEGFDFLHYDVKTGDFILHFKMNR | |
| | NLSFQRGLPGFMPAWDIVFEKNETQFDAKGTPFIAGKRIVPVIENHRFTGRYRDLYPAN | |
| | ELIALLEEKGIVERDGSNILPKLLENDDSHAIDTMVALIRSVLQMRNSNAATGEDYINS | |
| | PVRDLNGVCFDSRFQNPEWPMDADANGAYHIALKGQLLLNHLKESKDLKLQNGISNQDW | |
| | LAYIQELRN | |
| 194 | TQFEGFTNLYQVSKTLRFELIPQGKTLKHIQEQGFIEEDKARNDHYKELKPIIDRIYKT | WT AsCas12a |
| | YADQCLQLVQLDWENLSAAIDSYRKEKTEETRNALIEEQATYRNAIHDYFIGRTDNLTD | (AsCpf1) (aa) |
| | AINKRHAEIYKGLFKAELFNGKVLKQLGTVTTTEHENALLRSFDKFTTYFSGFYENRKN | |
| | VFSAEDISTAIPHRIVQDNFPKFKENCHIFTRLITAVPSLREHFENVKKAIGIFVSTSI | |
| | EEVFSFPFYNQLLTQTQIDLYNQLLGGISREAGTEKIKGLNEVLNLAIQKNDETAHIIA | |
| | SLPHRFIPLFKQILSDRNILSFILEEFKSDEEVIQSFCKYKTLLRNENVLETAEALENE | |
| | LNSIDLTHIFISHKKLETISSALCDHWDTLRNALYERRISELTGKITKSAKEKVQRSLK | |
| | HEDINLQEIISAAGKELSEAFKQKTSEILSHAHAALDQPLPTTLKKQEEKEILKSQLDS | |
| | LLGLYHLLDWFAVDESNEVDPEFSARLTGIKLEMEPSLSFYNKARNYATKKPYSVEKFK | |
| | LNFQMPTLASGWDVNKEKNNGAILFVKNGLYYLGIMPKQKGRYKALSFEPTEKTSEGED | |
| | KMYYDYFPDAAKMIPKCSTQLKAVTAHFQTHTTPILLSNNFIEPLEITKEIYDLNNPEK | |
| | EPKKFQTAYAKKTGDQKGYREALCKWIDFTRDFLSKYTKTTSIDLSSLRPSSQYKDLGE | |
| | YYAELNPLLYHISFQRIAEKEIMDAVETGKLYLFQIYNKDFAKGHHGKPNLHTLYWTGL | |
| | FSPENLAKTSIKLNGQAELFYRPKSRMKRMAHRLGEKMLNKKLKDQKTPIPDTLYQELY | |
| | DYVNHRLSHDLSDEARALLPNVITKEVSHEIIKDRRFTSDKFFFHVPITLNYQAANSPS | |
| | KFNQRVNAYLKEHPETPIIGIDRGERNLIYITVIDSTGKILEQRSLNTIQQFDYQKKLD | |
| | NREKERVAARQAWSVVGTIKDLKQGYLSQVIHEIVDLMIHYQAVVVLENLNFGFKSKRT | |
| | GIAEKAVYQQFEKMLIDKLNCLVLKDYPAEKVGGVLNPYQLTDQFTSFAKMGTQSGFLE | |
| | YVPAPYTSKIDPLTGFVDPFVWKTIKNHESRKHFLEGFDFLHYDVKTGDFILHFKMNRN | |
| | LSFQRGLPGFMPAWDIVFEKNETQFDAKGTPFIAGKRIVPVIENHRFTGRYRDLYPANE | |
| | LIALLEEKGIVERDGSNILPKLLENDDSHAIDTMVALIRSVLQMRNSNAATGEDYINSP | |
| | VRDLNGVCFDSRFQNPEWPMDADANGAYHIALKGQLLLNHLKESKDLKLQNGISNQDWL | |
| | AYIQELRN | |
| 195 | MPKPAVESEFSKVLKKHFPGERFRSSYMKRGGKILAAQGEEAVVAYLQGKSEEEPPNFQ | dCasPhi-2 (aa) |
| | PPAKCHVVTKSRDFAEWPIMKASEAIQRYIYALSTTERAACKPGKSSESHAAWFAATGV | |
| | SNHGYSHVQGLNLIFDHTLGRYDGVLKKVQLRNEKARARLESINASRADEGLPEIKAEE | |
| | EEVATNETGHLLQPPGINPSFYVYQTISPQAYRPRDEIVLPPEYAGYVRDPNAPIPLGV | |
| | VRNRCDIQKGCPGYIPEWQREAGTAISPKTGKAVTVPGLSPKKNKRMRRYWRSEKEKAQ | |
| | DALLVTVRIGTDWVVIDVRGLLRNARWRTIAPKDISLNALLDLFTGDPVIDVRRNIVTE | |
| | TYTLDACGTYARKWTLKGKQTKATLDKLTATQTVALVAIALGQTNPISAGISRVTQENG | |
| | ALQCEPLDRFTLPDDLLKDISAYRIAWDRNEEELRARSVEALPEAQQAEVRALDGVSKE | |
| | TARTQLCADFGLDPKRLPWDKMSSNTTFISEALLSNSVSRDQVFFTPAPKKGAKKKAPV | |
| | EVMRKDRTWARAYKPRLSVEAQKLKNEALWALKRTSPEYLKLSRRKEELCRRSINYVIE | |
| | KTRRRTQCQIVIPVIEDLNVRFFHGSGKRLPGWDNFFTAKKENRWFIQGLHKAFSDLRT | |
| | HRSFYVFEVRPERTSITCPKCGHCEVGNRDGEAFQCLSCGKTCNADLDVATHNLTQVAL | |
| | TGKTMPKREEPRDAQGTAPARKTKKASKSKAPPAEREDQTPAQEPSQTSGSGPKKKRKV | |
| | EDPKKKRKV | |
| 196 | PKPAVESEFSKVLKKHFPGERFRSSYMKRGGKILAAQGEEAVVAYLQGKSEEEPPNFQP | dCasPhi-2 (aa) |
| | PAKCHVVTKSRDFAEWPIMKASEAIQRYIYALSTTERAACKPGKSSESHAAWFAATGVS | |
| | NHGYSHVQGLNLIFDHTLGRYDGVLKKVQLRNEKARARLESINASRADEGLPEIKAEEE | |
| | EVATNETGHLLQPPGINPSFYVYQTISPQAYRPRDEIVLPPEYAGYVRDPNAPIPLGVV | |
| | RNRCDIQKGCPGYIPEWQREAGTAISPKTGKAVTVPGLSPKKNKRMRRYWRSEKEKAQD | |
| | ALLVTVRIGTDWVVIDVRGLLRNARWRTIAPKDISLNALLDLFTGDPVIDVRRNIVTFT | |
| | YTLDACGTYARKWTLKGKQTKATLDKLTATQTVALVAIALGQTNPISAGISRVTQENGA | |
| | LQCEPLDRFTLPDDLLKDISAYRIAWDRNEEELRARSVEALPEAQQAEVRALDGVSKET | |
| | ARTQLCADFGLDPKRLPWDKMSSNTTFISEALLSNSVSRDQVFFTPAPKKGAKKKAPVE | |
| | VMRKDRTWARAYKPRLSVEAQKLKNEALWALKRTSPEYLKLSRRKEELCRRSINYVIEK | |
| | TRRRTQCQIVIPVIEDLNVRFFHGSGKRLPGWDNFFTAKKENRWFIQGLHKAFSDLRTH | |
| | RSFYVFEVRPERTSITCPKCGHCEVGNRDGEAFQCLSCGKTCNADLDVATHNLTQVALT | |
| | GKTMPKREEPRDAQGTAPARKTKKASKSKAPPAEREDQTPAQEPSQTSGSGPKKKRKVE | |
| | DPKKKRKV | |
| 197 | MPKPAVESEFSKVLKKHFPGERFRSSYMKRGGKILAAQGEEAVVAYLQGKSEEEPPNFQ | WT CasPhi- |
| | PPAKCHVVTKSRDFAEWPIMKASEAIQRYIYALSTTERAACKPGKSSESHAAWFAATGV | 2 (aa) |
| | SNHGYSHVQGLNLIFDHTLGRYDGVLKKVQLRNEKARARLESINASRADEGLPEIKAEE | |
| | EEVATNETGHLLQPPGINPSFYVYQTISPQAYRPRDEIVLPPEYAGYVRDPNAPIPLGV | |
| | VRNRCDIQKGCPGYIPEWQREAGTAISPKTGKAVTVPGLSPKKNKRMRRYWRSEKEKAQ | |
| | DALLVTVRIGTDWVVIDVRGLLRNARWRTIAPKDISLNALLDLFTGDPVIDVRRNIVTF | |
| | TYTLDACGTYARKWTLKGKQTKATLDKLTATQTVALVAIDLGQTNPISAGISRVTQENG | |
| | ALQCEPLDRFTLPDDLLKDISAYRIAWDRNEEELRARSVEALPEAQQAEVRALDGVSKE | |
| | TARTQLCADFGLDPKRLPWDKMSSNTTFISEALLSNSVSRDQVFFTPAPKKGAKKKAPV | |
| | EVMRKDRTWARAYKPRLSVEAQKLKNEALWALKRISPEYLKLSRRKEELCRRSINYVIE | |
| | KTRRRTQCQIVIPVIEDLNVRFFHGSGKRLPGWDNFFTAKKENRWFIQGLHKAFSDLRT | |
| | HRSFYVFEVRPERTSITCPKCGHCEVGNRDGEAFQCLSCGKTCNADLDVATHNLTQVAL | |
| | TGKTMPKREEPRDAQGTAPARKTKKASKSKAPPAEREDQTPAQEPSQTS | |
| 198 | PKPAVESEFSKVLKKHFPGERFRSSYMKRGGKILAAQGEEAVVAYLQGKSEEEPPNFQP | WT CasPhi-2 |
| | PAKCHVVTKSRDFAEWPIMKASEAIQRYIYALSTTERAACKPGKSSESHAAWFAATGVS | (aa) |
| | NHGYSHVQGLNLIFDHTLGRYDGVLKKVQLRNEKARARLESINASRADEGLPEIKAEEE | |
| | EVATNETGHLLQPPGINPSFYVYQTISPQAYRPRDEIVLPPEYAGYVRDPNAPIPLGVV | |
| | RNRCDIQKGCPGYIPEWQREAGTAISPKTGKAVTVPGLSPKKNKRMRRYWRSEKEKAQD | |
| | ALLVTVRIGTDWVVIDVRGLLRNARWRTIAPKDISLNALLDLFTGDPVIDVRRNIVTFT | |
| | YTLDACGTYARKWTLKGKQTKATLDKLTATQTVALVAIDLGQTNPISAGISRVTQENGA | |
| | LQCEPLDRFTLPDDLLKDISAYRIAWDRNEEELRARSVEALPEAQQAEVRALDGVSKET | |
| | ARTQLCADFGLDPKRLPWDKMSSNTTFISEALLSNSVSRDQVFFTPAPKKGAKKKAPVE | |
| | VMRKDRTWARAYKPRLSVEAQKLKNEALWALKRTSPEYLKLSRRKEELCRRSINYVIEK | |
| | TRRRTQCQIVIPVIEDLNVRFFHGSGKRLPGWDNFFTAKKENRWFIQGLHKAFSDLRTH | |
| | RSFYVFEVRPERTSITCPKCGHCEVGNRDGEAFQCLSCGKTCNADLDVATHNLTQVALT | |
| | GKTMPKREEPRDAQGTAPARKTKKASKSKAPPAEREDQTPAQEPSQTS | |
| 199 | MAKNTITKTLKLRIVRPYNSAEVEKIVADEKNNREKIALEKNKDKVKEACSKHLKVAAY | WT |
| | CTTQVERNACLFCKARKLDDKFYQKLRGQFPDAVFWQEISEIFRQLQKQAAEIYNQSLI | Un1Cas12f1 |
| | ELYYEIFIKGKGIANASSVEHYLSDVCYTRAAELFKNAAIASGLRSKIKSNFRLKELKN | (aa) |
| | MKSGLPTTKSDNFPIPLVKQKGGQYTGFEISNHNSDFIIKIPFGRWQVKKEIDKYRPWE | |
| | KFDFEQVQKSPKPISLLLSTQRRKRNKGWSKDEGTEAEIKKVMNGDYQTSYIEVKRGSK | |
| | IGEKSAWMLNLSIDVPKIDKGVDPSIIGGIDVGVKSPLVCAINNAFSRYSISDNDLFHF | |
| | NKKMFARRRILLKKNRHKRAGHGAKNKLKPITILTEKSERFRKKLIERWACEIADFFIK | |
| | NKVGTVQMENLESMKRKEDSYFNIRLRGFWPYAEMQNKIEFKLKQYGIEIRKVAPNNTS | |
| | KTCSKCGHLNNYFNFEYRKKNKFPHFKCEKCNFKENADYNAALNISNPKLKSTKEEP | |
| 200 | AKNTITKTLKLRIVRPYNSAEVEKIVADEKNNREKIALEKNKDKVKEACSKHLKVAAYC | WT |
| | TTQVERNACLFCKARKLDDKFYQKLRGQFPDAVFWQEISEIFRQLQKQAAEIYNQSLIE | Un1Cas12f1 |
| | LYYEIFIKGKGIANASSVEHYLSDVCYTRAAELFKNAAIASGLRSKIKSNFRLKELKNM | (aa) |
| | KSGLPTTKSDNFPIPLVKQKGGQYTGFEISNHNSDFIIKIPFGRWQVKKEIDKYRPWEK | |
| | FDFEQVQKSPKPISLLLSTQRRKRNKGWSKDEGTEAEIKKVMNGDYQTSYIEVKRGSKI | |
| | GEKSAWMLNLSIDVPKIDKGVDPSIIGGIDVGVKSPLVCAINNAFSRYSISDNDLFHEN | |
| | KKMFARRRILLKKNRHKRAGHGAKNKLKPITILTEKSERFRKKLIERWACEIADFFIKN | |
| | KVGTVQMENLESMKRKEDSYFNIRLRGFWPYAEMQNKIEFKLKQYGIEIRKVAPNNTSK | |
| | TCSKCGHLNNYFNFEYRKKNKFPHFKCEKCNFKENADYNAALNISNPKLKSTKEEP | |
| 201 | MAKNTITKTLKLRIVRPYNSAEVEKIVADEKNNREKIALEKNKDKVKEACSKHLKVAAY | dUn1Cas12f1 |
| | CTTQVERNACLFCKARKLDDKFYQKLRGQFPDAVFWQEISEIFRQLQKQAAEIYNQSLI | (aa) |
| | ELYYEIFIKGKGIANASSVEHYLSDVCYTRAAELFKNAAIASGLRSKIKSNFRLKELKN | |
| | MKSGLPTTKSDNFPIPLVKQKGGQYTGFEISNHNSDFIIKIPFGRWQVKKEIDKYRPWE | |
| | KFDFEQVQKSPKPISLLLSTQRRKRNKGWSKDEGTEAEIKKVMNGDYQTSYIEVKRGSK | |
| | IGEKSAWMLNLSIDVPKIDKGVDPSIIGGIAVGVKSPLVCAINNAFSRYSISDNDLFHF | |
| | NKKMFARRRILLKKNRHKRAGHGAKNKLKPITILTEKSERFRKKLIERWACEIADFFIK | |
| | NKVGTVQMENLESMKRKEDSYFNIRLRGFWPYAEMQNKIEFKLKQYGIEIRKVAPNNTS | |
| | KTCSKCGHLNNYFNFEYRKKNKFPHFKCEKCNFKENAAYNAALNISNPKLKSTKEEP | |
| 202 | AKNTITKTLKLRIVRPYNSAEVEKIVADEKNNREKIALEKNKDKVKEACSKHLKVAAYC | dUn1Cas12f1 |
| | TTQVERNACLFCKARKLDDKFYQKLRGQFPDAVFWQEISEIFRQLQKQAAEIYNQSLIE | (aa) |
| | LYYEIFIKGKGIANASSVEHYLSDVCYTRAAELFKNAAIASGLRSKIKSNFRLKELKNM | |
| | KSGLPTTKSDNFPIPLVKQKGGQYTGFEISNHNSDFIIKIPFGRWQVKKEIDKYRPWEK | |
| | FDFEQVQKSPKPISLLLSTQRRKRNKGWSKDEGTEAEIKKVMNGDYQTSYIEVKRGSKI | |
| | GEKSAWMLNLSIDVPKIDKGVDPSIIGGIAVGVKSPLVCAINNAFSRYSISDNDLFHEN | |
| | KKMFARRRILLKKNRHKRAGHGAKNKLKPITILTEKSERFRKKLIERWACEIADFFIKN | |
| | KVGTVQMENLESMKRKEDSYFNIRLRGFWPYAEMQNKIEFKLKQYGIEIRKVAPNNTSK | |
| | TCSKCGHLNNYFNFEYRKKNKFPHFKCEKCNFKENAAYNAALNISNPKLKSTKEEP | |
| 203 | MARILAFAIGISSIGWAFSENDELKDCGVRIFTKVENPKTGESLALPRRLARSARKRLA | dCjCas9 (aa) |
| | RRKARLNHLKHLIANEFKLNYEDYQSFDESLAKAYKGSLISPYELRFRALNELLSKQDE | |
| | ARVILHIAKRRGYDDIKNSDDKEKGAILKAIKQNEEKLANYQSVGEYLYKEYFQKFKEN | |
| | SKEFTNVRNKKESYERCIAQSFLKDELKLIFKKQREFGFSFSKKFEEEVLSVAFYKRAL | |
| | KDFSHLVGNCSFFTDEKRAPKNSPLAFMFVALTRIINLLNNLKNTEGILYTKDDLNALL | |
| | NEVLKNGTLTYKQTKKLLGLSDDYEFKGEKGTYFIEFKKYKEFIKALGEHNLSQDDLNE | |
| | IAKDITLIKDEIKLKKALAKYDLNQNQIDSLSKLEFKDHLNISFKALKLVTPLMLEGKK | |
| | YDEACNELNLKVAINEDKKDELPAFNETYYKDEVTNPVVLRAIKEYRKVLNALLKKYGK | |
| | VHKINIELAREVGKNHSQRAKIEKEQNENYKAKKDAELECEKLGLKINSKNILKLRLFK | |
| | EQKEFCAYSGEKIKISDLQDEKMLEIDAIYPYSRSFDDSYMNKVLVFTKQNQEKLNQTP | |
| | FEAFGNDSAKWQKIEVLAKNLPTKKQKRILDKNYKDKEQKNFKDRNLNDTRYIARLVLN | |
| | YTKDYLDFLPLSDDENTKLNDTQKGSKVHVEAKSGMLTSALRHTWGFSAKDRNNHLHHA | |
| | IDAVIIAYANNSIVKAFSDFKKEQESNSAELYAKKISELDYKNKRKFFEPFSGFRQKVL | |
| | DKIDEIFVSKPERKKPSGALHEETFRKEEEFYQSYGGKEGVLKALELGKIRKVNGKIVK | |
| | NGDMFRVDIFKHKKTNKFYAVPIYTMDFALKVLPNKAVARSKKGEIKDWILMDENYEFC | |
| | FSLYKDSLILIQTKDMQEPEFVYYNAFTSSTVSLIVSKHDNKFETLSKNQKILFKNANE | |
| | KEVIAKSIGIQNLKVFEKYIVSALGEVTKAEFRQREDFKK | |
| 204 | ARILAFAIGISSIGWAFSENDELKDCGVRIFTKVENPKTGESLALPRRLARSARKRLAR | dCjCas9 (aa) |
| | RKARLNHLKHLIANEFKLNYEDYQSFDESLAKAYKGSLISPYELRFRALNELLSKQDFA | |
| | RVILHIAKRRGYDDIKNSDDKEKGAILKAIKQNEEKLANYQSVGEYLYKEYFQKFKENS | |
| | KEFTNVRNKKESYERCIAQSFLKDELKLIFKKQREFGFSFSKKFEEEVLSVAFYKRALK | |
| | DFSHLVGNCSFFTDEKRAPKNSPLAFMFVALTRIINLLNNLKNTEGILYTKDDLNALLN | |
| | EVLKNGTLTYKQTKKLLGLSDDYEFKGEKGTYFIEFKKYKEFIKALGEHNLSQDDLNEI | |
| | AKDITLIKDEIKLKKALAKYDLNQNQIDSLSKLEFKDHLNISFKALKLVTPLMLEGKKY | |
| | DEACNELNLKVAINEDKKDFLPAFNETYYKDEVTNPVVLRAIKEYRKVLNALLKKYGKV | |
| | HKINIELAREVGKNHSQRAKIEKEQNENYKAKKDAELECEKLGLKINSKNILKLRLFKE | |
| | QKEFCAYSGEKIKISDLQDEKMLEIDAIYPYSRSFDDSYMNKVLVFTKQNQEKLNQTPF | |
| | EAFGNDSAKWQKIEVLAKNLPTKKQKRILDKNYKDKEQKNFKDRNLNDTRYIARLVLNY | |
| | TKDYLDFLPLSDDENTKLNDTQKGSKVHVEAKSGMLTSALRHTWGFSAKDRNNHLHHAI | |
| | DAVIIAYANNSIVKAFSDFKKEQESNSAELYAKKISELDYKNKRKFFEPFSGFRQKVLD | |
| | KIDEIFVSKPERKKPSGALHEETFRKEEEFYQSYGGKEGVLKALELGKIRKVNGKIVKN | |
| | GDMFRVDIFKHKKTNKFYAVPIYTMDFALKVLPNKAVARSKKGEIKDWILMDENYEFCF | |
| | SLYKDSLILIQTKDMQEPEFVYYNAFTSSTVSLIVSKHDNKFETLSKNQKILFKNANEK | |
| | EVIAKSIGIQNLKVFEKYIVSALGEVTKAEFRQREDFKK | |
| 205 | MSSGSGHMARILAFDIGISSIGWAFSENDELKDCGVRIFTKVENPKTGESLALPRRLAR | WT CjCas9 |
| | SARKRLARRKARLNHLKHLIANEFKLNYEDYQSFDESLAKAYKGSLISPYELRFRALNE | (aa) |
| | LLSKQDFARVILHIAKRRGYDDIKNSDDKEKGAILKAIKQNEEKLANYQSVGEYLYKEY | |
| | FQKFKENSKEFTNVRNKKESYERCIAQSFLKDELKLIFKKQREFGFSFSKKFEEEVLSV | |
| | AFYKRALKDFSHLVGNCSFFTDEKRAPKNSPLAFMFVALTRIINLLNNLKNTEGILYTK | |
| | DDLNALLNEVLKNGTLTYKQTKKLLGLSDDYEFKGEKGTYFIEFKKYKEFIKALGEHNL | |
| | SQDDLNEIAKDITLIKDEIKLKKALAKYDLNQNQIDSLSKLEFKDHLNISFKALKLVTP | |
| | LMLEGKKYDEACNELNLKVAINEDKKDFLPAFNETYYKDEVINPVVLRAIKEYRKVLNA | |
| | LLKKYGKVHKINIELGGGSGGYIARLVLNYTKDYLDFLPLSDDENTKLNDTQKGSKVHV | |
| | EAKSGMLTSALRHTWGFSAKDRNNHLHHAIDAVIIAYANNSIVKAFSDFKKEQESNSAE | |
| | LYAKKISELDYKNKRKFFEPFSGFRQKVLDKIDEIFVSKPERKKPSGALHEETFRKEEE | |
| | FYQSYGGKEGVLKALELGKIRKVNGKIVKNGDMFRVDIFKHKKINKFYAVPIYTMDFAL | |
| | KVLPNKAVARSKKGEIKDWILMDENYEFCFSLYKDSLILIQTKDMQEPEFVYYNAFTSS | |
| | TVSLIVSKHDNKFETLSKNQKILFKNANEKEVIAKSIGIQNLKVFEKYIVSALGEVTKA | |
| | EFRQREDFKK | |
| 206 | SSGSGHMARILAFDIGISSIGWAFSENDELKDCGVRIFTKVENPKTGESLALPRRLARS | WT |
| | ARKRLARRKARLNHLKHLIANEFKLNYEDYQSFDESLAKAYKGSLISPYELRFRALNEL | CjCas9 (aa) |
| | LSKQDFARVILHIAKRRGYDDIKNSDDKEKGAILKAIKQNEEKLANYQSVGEYLYKEYF | |
| | QKFKENSKEFTNVRNKKESYERCIAQSFLKDELKLIFKKQREFGFSFSKKFEEEVLSVA | |
| | FYKRALKDFSHLVGNCSFFTDEKRAPKNSPLAFMFVALTRIINLLNNLKNTEGILYTKD | |
| | DLNALLNEVLKNGTLTYKQTKKLLGLSDDYEFKGEKGTYFIEFKKYKEFIKALGEHNLS | |
| | QDDLNEIAKDITLIKDEIKLKKALAKYDLNQNQIDSLSKLEFKDHLNISFKALKLVTPL | |
| | MLEGKKYDEACNELNLKVAINEDKKDFLPAFNETYYKDEVTNPVVLRAIKEYRKVLNAL | |
| | LKKYGKVHKINIELGGGSGGYIARLVLNYTKDYLDFLPLSDDENTKLNDTQKGSKVHVE | |
| | AKSGMLTSALRHTWGFSAKDRNNHLHHAIDAVIIAYANNSIVKAFSDFKKEQESNSAEL | |
| | YAKKISELDYKNKRKFFEPFSGFRQKVLDKIDEIFVSKPERKKPSGALHEETFRKEEEF | |
| | YQSYGGKEGVLKALELGKIRKVNGKIVKNGDMFRVDIFKHKKTNKFYAVPIYTMDFALK | |
| | VLPNKAVARSKKGEIKDWILMDENYEFCFSLYKDSLILIQTKDMQEPEFVYYNAFTSST | |
| | VSLIVSKHDNKFETLSKNQKILFKNANEKEVIAKSIGIQNLKVFEKYIVSALGEVTKAE | |
| | FRQREDFKK | |
| 207 | MSSTSGKNPTNPIVRTISCNLSAKEDVLRKVWEEMSQKNTPLIVQLLKSVSEQPEFEAN | WT Cas12k |
| | KENGTITKKEITELRRDITEDSDLKKQSGRLRSSADSLVTEVYSSWLRLYQVRKNKKEG | (aa) |
| | KEYFLNNILKSDVELVEQSNCDLQTIRCKAKEILSQVEEFIEQVNNKPKINKTTSAKKK | |
| | INKSNKNNKAIEEILNRFFIGNIDKTLINTLYEIHRKSPDILTQCAVAYLIKNDNKVSE | |
| | AEENLTQLNKRSSAKEIEIKRLETQIQNTRLPNGRDITGEKYSQAFEKLVNQVPQDNEE | |
| | FAEWIAILLKKVSSLPYPILYSSGDLSWYKDEKGNIFVYFNGWAEYHFQICCDKRQLRF | |
| | FERFLKYYKALKASEKGEEKLSGSLVTLRSAHLLWRQGKGKGEPWKVNKLALHCTYDAR | |
| | LWTAEGTEEVRQEKTDKAQAEVNQAESNENIDSKQQKKLTKNKSSLSRLKNSFARPSKP | |
| | LYRGQSNIIVGISFHPVELATLVVVDINTKEILICKTVKELLGDAFPLLSRRRRQQVHF | |
| | RKEREKAQKKDSPCDLGESKLGEYVDRLLAKRIVEVAKEYQASCIVLPGLKGIREIRTS | |
| | VIQAKAETKFPGDINAQELYVKEYNRQIHNWSYSRLQESIKSRAAELKISIKFGKQPSH | |
| | STLQEQAINLALSA | |
| 208 | SSTSGKNPTNPIVRTISCNLSAKEDVLRKVWEEMSQKNTPLIVQLLKSVSEQPEFEANK | WT Cas12k |
| | ENGTITKKEITELRRDITEDSDLKKQSGRLRSSADSLVTEVYSSWLRLYQVRKNKKEGK | (aa) |
| | EYFLNNILKSDVELVEQSNCDLQTIRCKAKEILSQVEEFIEQVNNKPKINKTTSAKKKI | |
| | NKSNKNNKAIEEILNRFFIGNIDKTLTNTLYEIHRKSPDILTQCAVAYLIKNDNKVSEA | |
| | EENLTQLNKRSSAKEIEIKRLETQIQNTRLPNGRDITGEKYSQAFEKLVNQVPQDNEEF | |
| | AEWIAILLKKVSSLPYPILYSSGDLSWYKDEKGNIFVYFNGWAEYHFQICCDKRQLRFF | |
| | ERFLKYYKALKASEKGEEKLSGSLVTLRSAHLLWRQGKGKGEPWKVNKLALHCTYDARL | |
| | WTAEGTEEVRQEKTDKAQAEVNQAESNENIDSKQQKKLTKNKSSLSRLKNSFARPSKPL | |
| | YRGQSNIIVGISFHPVELATLVVVDINTKEILICKTVKELLGDAFPLLSRRRRQQVHER | |
| | KEREKAQKKDSPCDLGESKLGEYVDRLLAKRIVEVAKEYQASCIVLPGLKGIREIRTSV | |
| | IQAKAETKFPGDINAQELYVKEYNRQIHNWSYSRLQESIKSRAAELKISIKFGKQPSHS | |
| | TLQEQAINLALSA | |
| 209 | MGPKKKRKVGSGSAKNTITKTLKLRIVRPYNSAEVEKIVADEKNNREKIALEKNKDKVK | CasMini |
| | EACSKHLKVAAYCTTQVERNACLFCKARKLDDKFYQKLRGQFPDAVEWQEISEIFRQLQ | |
| | KQAAEIYNQSLIELYYEIFIKGKGIANASSVEHYLSRVCYRRAAELFKNAAIASGLRSK | |
| | IKSNFRLKELKNMKSGLPTTKSDNFPIPLVKQKGGQYTGFEISNHNSDFIIKIPFGRWQ | |
| | VKKEIDKYRPWEKFDFEQVQKSPKPISLLLSTQRRKRNKGWSKDEGTEAEIKKVMNGDY | |
| | QTSYIEVKRGSKICEKSAWMLNLSIDVPKIDKGVDPSIIGGIAVGVRSPLVCAINNAFS | |
| | RYSISDNDLFHFNKKMFARRRILLKKNRHKRAGHGAKNKLKPITILTEKSERFRKKLIE | |
| | RWACEIADFFIKNKVGTVQMENLESMKRKEDSYFNIRLRGFWPYAEMQNKIEFKLKQYG | |
| | IEIRKVAPNNTSKTCSKCGHLNNYFNFEYRKKNKFPHFKCEKCNFKENAAYNAALNISN | |
| | PKLKSTKERPAKRPAATKKAGQAKKKK | |
| 210 | GPKKKRKVGSGSAKNTITKTLKLRIVRPYNSAEVEKIVADEKNNREKIALEKNKDKVKE | CasMini |
| | ACSKHLKVAAYCTTQVERNACLFCKARKLDDKFYQKLRGQFPDAVFWQEISEIFRQLQK | |
| | QAAEIYNQSLIELYYEIFIKGKGIANASSVEHYLSRVCYRRAAELFKNAAIASGLRSKI | |
| | KSNFRLKELKNMKSGLPTTKSDNFPIPLVKQKGGQYTGFEISNHNSDFIIKIPFGRWQV | |
| | KKEIDKYRPWEKFDFEQVQKSPKPISLLLSTQRRKRNKGWSKDEGTEAEIKKVMNGDYQ | |
| | TSYIEVKRGSKICEKSAWMLNLSIDVPKIDKGVDPSIIGGIAVGVRSPLVCAINNAFSR | |
| | YSISDNDLFHFNKKMFARRRILLKKNRHKRAGHGAKNKLKPITILTEKSERFRKKLIER | |
| | WACEIADFFIKNKVGTVQMENLESMKRKEDSYFNIRLRGFWPYAEMQNKIEFKLKQYGI | |
| | EIRKVAPNNTSKTCSKCGHLNNYFNFEYRKKNKFPHFKCEKCNFKENAAYNAALNISNP | |
| | KLKSTKERPAKRPAATKKAGQAKKKK | |
| 211 | gaaacaccguaauuucuacucuuguagau | dAsCas12a |
| | | gRNA scaffold |
| 212 | CAACGAUUGCCCCUCACGAGGGGAC | dCasPhi-2 |
| | | gRNA scaffold |
| 213 | ACCGCUUCACUUAGAGUGAAGGUGGGCUGCUUGCAUCAGCCUAAUGUCGAGAAGUGCUU | Un1Cas12f1 |
| | UCUUCGGAAAGUAACCCUCGAAACAAAGAAA | gRNA scaffold |
| 214 | GGAAUGAAC | Un1Cas12f1 |
| | | gRNA scaffold |
| 215 | UUUUAUUUU | Un1Cas12f1 |
| | | gRNA scaffold |
| 216 | GCGGUUUUAGGGGAUUGUAACCCCGCAGAGUCCCGCAAACUCUUUAUUUUAGUCCCUUU | CjCas9 gRNA |
| | UCAGGGACUAAAAC | scaffold |
| 217 | AUAUUAAUAGCGCCGCAAUUCAUGCUGCUUGCAGCCUCUGAAUUUUGUUAAAUGAGGGU | Cas12k gRNA |
| | UAGUUUGACUGUAUAAAUACAGUCUUGCUUUCUGACCCUGGUAGCUGCUCACCCUGAUG | scaffold |
| | CUGCUGUCAAUAGACAGGAUAGGUGCGCUCCCAGCAAUAAGGGCGCGGAUGUACUGCUG | |
| | UAGUGGCUACUGAAUCACCCCCGAUCAAGGGGGAACCCUCCAAAAGGUGGGUUGAAAG | |
| 218 | GGGCUUCACUGAUAAAGUGGAGAACCGCUUCACCAAAAGCUGUCCCUUAGGGGAUUAGA | CasMini gRNA |
| | ACUUGAGUGAAGGUGGGCUGCUUGCAUCAGCCUAAUGUCGAGAAGUGCUUUCUUCGGAA | scaffold |
| | AGUAACCCUCGAAACAAAUUCAUUUGAAUGAAGGAAUGCAAC | |
| 219 | GSGSG | linker (aa) |
| 220 | MPKPAVESEFSKVLKKHFPGERFRSSYMKRGGKILAAQGEEAVVAYLQGKSEEEPPNFQ | dCasPhi-2 |
| | PPAKCHVVTKSRDFAEWPIMKASEAIQRYIYALSTTERAACKPGKSSESHAAWFAATGV | |
| | SNHGYSHVQGLNLIFDHTLGRYDGVLKKVQLRNEKARARLESINASRADEGLPEIKAEE | |
| | EEVATNETGHLLQPPGINPSFYVYQTISPQAYRPRDEIVLPPEYAGYVRDPNAPIPLGV | |
| | VRNRCDIQKGCPGYIPEWQREAGTAISPKTGKAVTVPGLSPKKNKRMRRYWRSEKEKAQ | |
| | DALLVTVRIGTDWVVIDVRGLLRNARWRTIAPKDISLNALLDLFTGDPVIDVRRNIVTF | |
| | TYTLDACGTYARKWTLKGKQTKATLDKLTATQTVALVAIALGQTNPISAGISRVTQENG | |
| | ALQCEPLDRFTLPDDLLKDISAYRIAWDRNEEELRARSVEALPEAQQAEVRALDGVSKE | |
| | TARTQLCADFGLDPKRLPWDKMSSNTTFISEALLSNSVSRDQVFFTPAPKKGAKKKAPV | |
| | EVMRKDRTWARAYKPRLSVEAQKLKNEALWALKRISPEYLKLSRRKEELCRRSINYVIE | |
| | KTRRRTQCQIVIPVIEDLNVRFFHGSGKRLPGWDNFFTAKKENRWFIQGLHKAFSDLRT | |
| | HRSFYVFEVRPERTSITCPKCGHCEVGNRDGEAFQCLSCGKTCNADLDVATHNLTQVAL | |
| | TGKTMPKREEPRDAQGTAPARKTKKASKSKAPPAEREDQTPAQEPSQTS | |
| 221 | PKPAVESEFSKVLKKHFPGERFRSSYMKRGGKILAAQGEEAVVAYLQGKSEEEPPNFQP | dCasPhi-2 |
| | PAKCHVVTKSRDFAEWPIMKASEAIQRYIYALSTTERAACKPGKSSESHAAWFAATGVS | |
| | NHGYSHVQGLNLIFDHTLGRYDGVLKKVQLRNEKARARLESINASRADEGLPEIKAEEE | |
| | EVATNETGHLLQPPGINPSFYVYQTISPQAYRPRDEIVLPPEYAGYVRDPNAPIPLGVV | |
| | RNRCDIQKGCPGYIPEWQREAGTAISPKTGKAVTVPGLSPKKNKRMRRYWRSEKEKAQD | |
| | ALLVTVRIGTDWVVIDVRGLLRNARWRTIAPKDISLNALLDLFTGDPVIDVRRNIVTFT | |
| | YTLDACGTYARKWTLKGKQTKATLDKLTATQTVALVAIALGQTNPISAGISRVTQENGA | |
| | LQCEPLDRFTLPDDLLKDISAYRIAWDRNEEELRARSVEALPEAQQAEVRALDGVSKET | |
| | ARTQLCADFGLDPKRLPWDKMSSNTTFISEALLSNSVSRDQVFFTPAPKKGAKKKAPVE | |
| | VMRKDRTWARAYKPRLSVEAQKLKNEALWALKRISPEYLKLSRRKEELCRRSINYVIEK | |
| | TRRRTQCQIVIPVIEDLNVRFFHGSGKRLPGWDNFFTAKKENRWFIQGLHKAFSDLRTH | |
| | RSFYVFEVRPERTSITCPKCGHCEVGNRDGEAFQCLSCGKTCNADLDVATHNLTQVALT | |
| | GKTMPKREEPRDAQGTAPARKTKKASKSKAPPAEREDQTPAQEPSQTS | |
| 222 | NNNNGNNN,whereNisanynucleotide | N. meningitidis |
| | | Cas9 PAM |
| 223 | AKNTITKTLKLRIVRPYNSAEVEKIVADEKNNREKIALEKNKDKVKEACSKHLKVAAYC | CasMini |
| | TTQVERNACLFCKARKLDDKFYQKLRGQFPDAVFWQEISEIFRQLQKQAAEIYNQSLIE | Cas12f/D326A/ |
| | LYYEIFIKGKGIANASSVEHYLSRVCYRRAAELFKNAAIASGLRSKIKSNFRLKELKNM | D510A/D143R/ |
| | KSGLPTTKSDNFPIPLVKQKGGQYTGFEISNHNSDFIIKIPFGRWQVKKEIDKYRPWEK | T147R/K330R/ |
| | FDFEQVQKSPKPISLLLSTQRRKRNKGWSKDEGTEAEIKKVMNGDYQTSYIEVKRGSKI | E528R (aa) |
| | CEKSAWMLNLSIDVPKIDKGVDPSIIGGIAVGVRSPLVCAINNAFSRYSISDNDLFHEN | |
| | KKMFARRRILLKKNRHKRAGHGAKNKLKPITILTEKSERFRKKLIERWACEIADFFIKN | |
| | KVGTVQMENLESMKRKEDSYFNIRLRGFWPYAEMQNKIEFKLKQYGIEIRKVAPNNTSK | |
| | TCSKCGHLNNYFNFEYRKKNKFPHFKCEKCNFKENAAYNAALNISNPKLKSTKERP | |
| 224 | TBN, where N is any nucleotide, and B is G, T, or C | PAM-CasPhi |
| 225 | TTTN, where N is any nucleotide | PAM- |
| | | Un1Cas12f1 |
| 226 | NNNNACAC, where N is any nucleotide | PAM-CjCas9 |
| 227 | GGTT | PAM-Cas12k |
| 228 | TTTR, where R is G or A | PAM-Cas12f |
| 229 | TGGAAGTAAGGCAAAAAGATTGC | Cas12a gRNA |
| | | 1 spacer |
| 230 | AGGCAAAAAGATTGCTTTGAATA | Cas12a gRNA |
| | | 2 spacer |
| 231 | AGTGGTTCTCAACCAGGGGCAAT | Cas12a gRNA |
| | | 3 spacer |
| 232 | CAATTTTACTCCTTAGGGGACCT | Cas12a gRNA |
| | | 4 spacer |
| 233 | TTTTCACAATGTCTGGAGACATT | Cas12a gRNA |
| | | 5 spacer |
| 234 | TGGGAAATGCATCATTAGGTGAT | Cas12a gRNA |
| | | 6 spacer |
| 235 | AATTGTACTACAGTAGTTAAGTA | Cas12a gRNA |
| | | 7 spacer |
| 236 | AGTATGGTAACATGCTGTACAGG | Cas12a gRNA |
| | | 8 spacer |
| 237 | TATAGTAGGCTAGACCATCTAGC | Cas12a gRNA |
| | | 9 spacer |
| 238 | TCACAAAATCACCTAATGATGCA | Cas12a gRNA |
| | | 10 spacer |
| 239 | TTGTCCCATGCATTGTAAGGTGG | Cas12a gRNA |
| | | 11 spacer |
| 240 | GAGAACCACTATTCTGGTCTAAC | Cas12a gRNA |
| | | 12 spacer |
| 241 | AATTAAATATTCAAAGCAATCTT | Cas12a gRNA |
| | | 13 spacer |
| 242 | ATCTTTTTGCCTTACTTCCAAGT | Cas12a gRNA |
| | | 14 spacer |
| 243 | CTTACTTCCAAGTTTTAGATAAT | Cas12a gRNA |
| | | 15 spacer |
| 244 | GCTCACGCCTGTCGTCTCAGCAC | Cas12a gRNA |
| | | 16 spacer |
| 245 | GTGAGGCCCAGGCGGGCAGATCG | Cas12a gRNA |
| | | 17 spacer |
| 246 | CAGCAGCTCCCAAGTTCCTCCTG | Cas12a gRNA |
| | | 18 spacer |
| 247 | CCCAAGTTCCTCCTGTTTAGAAT | Cas12a gRNA |
| | | 19 spacer |
| 248 | TAATATTTTCAAGGCTGGATTTT | Cas12a gRNA |
| | | 20 spacer |
| 249 | CTGGATTTTTTTGAACGAAATGC | Cas12a gRNA |
| | | 21 spacer |
| 250 | TAGCTATTCTGCAGCTCTGAAAG | Cas12a gRNA |
| | | 22 spacer |
| 251 | TTTCACCTCGTTCCAGGAAAGCA | Cas12a gRNA |
| | | 23 spacer |
| 252 | TCACTGCAAGCTCTGCCTCCCGG | Cas12a gRNA |
| | | 24 spacer |
| 253 | CCGCCACCATGCCTGGCTAATTT | Cas12a gRNA |
| | | 25 spacer |
| 254 | ACACCCAGCTAGTTTTTGTATTT | Cas12a gRNA |
| | | 26 spacer |
| 255 | TTGTATTTTTTGTAGAAAGGGGG | Cas12a gRNA |
| | | 27 spacer |
| 256 | AAGTAAGGCAAAAAGATTGC | gRNA V |
| | | spacer |
| 257 | CAATTTTACTCCTTAGGGGA | gRNA W |
| | | spacer |
| 258 | GAGAGCAGATACTACTGCCA | gRNA X |
| | | spacer |
| 259 | TGGAGACCAAGAATGCTGCT | gRNA Y |
| | | spacer |
| 260 | TGATTTTGTGATCTTGCGAA | gRNA Z spacer |
| 261 | TAACATGCTGTACAGGTTTA | gRNA AA |
| | | spacer |
| 262 | CTAGACCATCTAGCTTTGTG | gRNA AB |
| | | spacer |
| 263 | TCTCCACCCACTAGATGGCA | gRNA AC |
| | | spacer |
| 264 | TCCCCTAAGGAGTAAAATTG | gRNA AD |
| | | spacer |
| 265 | TCTAACTTTGAATTAAATAT | gRNA AE |
| | | spacer |

1. A DNA-targeting system comprising:
(a) a DNA-targeting domain that binds to a target site in a regulatory DNA element of a frataxin (FXN) locus; and
(b) at least one effector domain that increases transcription of the FXN locus.

2. A DNA-targeting system comprising a DNA-targeting domain that binds to a target site in an enhancer of a frataxin (FXN) locus.

3. The DNA-targeting system of claim 2, further comprising at least one effector domain that increases transcription of the FXN locus.

4. A DNA-targeting system comprising:
(a) a DNA-targeting domain that binds to a target site in an enhancer of a frataxin (FXN) locus; and
(b) at least one effector domain that increases transcription of the FXN locus.

5. The DNA-targeting system of any of claims 1-4, wherein binding of the DNA-targeting domain to the target site does not introduce a genetic disruption or a DNA break at or near the target site.

6. The DNA-targeting system of any of claims 1-5, wherein the DNA-targeting domain comprises a Clustered Regularly Interspaced Short Palindromic Repeats associated (Cas)-guide RNA (gRNA) combination comprising (a) a Cas protein or a variant thereof, optionally wherein the Cas protein or a variant thereof is a deactivated Cas (dCas) protein, and (b) at least one gRNA; a zinc finger protein (ZFP); a transcription activator-like effector (TALE); a meganuclease; a homing endonuclease; or an I-SceI enzyme or a variant thereof, optionally wherein the DNA-targeting domain comprises a catalytically inactive variant of any of the foregoing.

7. A DNA-targeting system comprising a DNA-targeting domain that is a Cas-guide RNA (gRNA) combination comprising:
(a) a deactivated Cas (dCas) protein;
(b) at least one effector domain that increases transcription of a frataxin (FXN) locus; and
(c) at least one gRNA comprising a gRNA spacer sequence that is capable of hybridizing to a target site in a regulatory DNA element of the FXN locus or is complementary to the target site.

8. The DNA-targeting system of claim 6 or 7, wherein at least one gRNA is capable of complexing with the Cas protein or variant thereof or the dCas protein.

9. The DNA-targeting system of any of claims 6-8, wherein at least one gRNA comprises a gRNA spacer sequence that is capable of hybridizing to the target site or is complementary to the target site.

10. The DNA-targeting system of any of claims 6-9, wherein the Cas protein or a variant thereof is a deactivated Cas9 (dCas9) protein, optionally a Staphylococcus aureus dCas9 (dSaCas9) protein or a Streptococcus pyogenes dCas9 (dS9Cas9) protein.

11. A DNA-targeting system comprising a DNA-targeting domain that is a Cas-guide RNA (gRNA) combination comprising:
(a) Staphylococcus aureus dCas9 protein (dSaCas9);
(b) at least one effector domain that increases transcription of a frataxin (FXN) locus; and
(c) at least one gRNA, comprising a gRNA spacer sequence that is capable of hybridizing to a target site in a regulatory DNA element of the FXN locus or is complementary to the target site.

12. A DNA-targeting system comprising a DNA-targeting domain that is a Cas-guide RNA (gRNA) combination comprising:
(a) Staphylococcus aureus dCas9 protein (dSaCas9); and
(b) at least one gRNA, comprising a gRNA spacer sequence that is capable of hybridizing to a target site in an enhancer of a frataxin (FXN) locus or is complementary to the target site.

13. The DNA-targeting system of claim 12, further comprising at least one effector domain that increases transcription of the FXN locus.

14. A DNA-targeting system comprising a DNA-targeting domain that is a Cas-guide RNA (gRNA) combination comprising:
(a) Staphylococcus aureus dCas9 protein (dSaCas9);
(b) at least one effector domain that increases transcription of a frataxin (FXN) locus; and
(c) at least one gRNA, comprising a gRNA spacer sequence that is capable of hybridizing to a target site in an enhancer of the FXN locus or is complementary to the target site.

15. The DNA-targeting system of any of claims 6-14, wherein the Cas protein or a variant thereof is a Staphylococcus aureus dCas9 protein (dSaCas9) that comprises at least one amino acid mutation selected from D10A and N580A, with reference to numbering of positions of SEQ ID NO:73, and/or the Cas protein or a variant thereof comprises the sequence set forth in SEQ ID NO:72, or an amino acid sequence that has at least 90%, 91%, 92%, 93%, 94%, 95%, 96%, 97%, 98%, or 99% sequence identity thereto.

16. The DNA-targeting system of any of claims 6-10, wherein the Cas protein or a variant thereof is a Streptococcus pyogenes dCas9 (dSpCas9) protein that comprises at least one amino acid mutation selected from D10A and H840A, with reference to numbering of positions of SEQ ID NO:79, and/or the Cas protein or a variant thereof comprises the sequence set forth in SEQ ID NO:78, or an amino acid sequence that has at least 90%, 91%, 92%, 93%, 94%, 95%, 96%, 97%, 98%, or 99% sequence identity thereto.

17. The DNA-targeting system of any of claims 6-16, wherein the Cas protein or a variant thereof is a split variant Cas protein, wherein the split variant Cas protein comprises a first polypeptide comprising an N-terminal fragment of the variant Cas protein and an N-terminal Intein, and a second polypeptide comprising a C-terminal fragment of the variant Cas protein and a C-terminal Intein, wherein when the first polypeptide and the second polypeptide of the split variant Cas protein are present in proximity or present in the same cell, the N-terminal Intein and C-terminal Intein self-excise and ligate the N-terminal fragment and the C-terminal fragment of the variant Cas protein to form a full-length variant Cas protein.

18. The DNA-targeting system of claim 17, wherein:
the N-terminal Intein comprises an N-terminal Npu Intein, or the sequence set forth in SEQ ID NO:178, or an amino acid sequence that has at least 90%, 91%, 92%, 93%, 94%, 95%, 96%, 97%, 98%, or 99% sequence identity thereto, or a portion of any of the foregoing; and
the N-terminal fragment of the variant Cas protein comprises:
the N-terminal fragment of variant SpCas9 from the N-terminal end up to position 573 of the dSpCas9 sequence set forth in SEQ ID NO:78, or an amino acid sequence that has at least 90%, 91%, 92%, 93%, 94%, 95%, 96%, 97%, 98%, or 99% sequence identity thereto; or
the sequence set forth in SEQ ID NO:176, or an amino acid sequence that has at least 90%, 91%, 92%, 93%, 94%, 95%, 96%, 97%, 98%, or 99% sequence identity thereto, or a portion of any of the foregoing; and/or wherein:
the C-terminal Intein comprises a C-terminal Npu Intein, or the sequence set forth in SEQ ID NO:182, or an amino acid sequence that has at least 90%, 91%, 92%, 93%, 94%, 95%, 96%, 97%, 98%, or 99% sequence identity thereto, or a portion of any of the foregoing; and
the C-terminal fragment of the variant Cas protein comprises:
the C-terminal fragment of variant SpCas9 from position 574 to the C-terminal end of the dSpCas9 sequence set forth in SEQ ID NO:78, or an amino acid sequence that has at least 90%, 91%, 92%, 93%, 94%, 95%, 96%, 97%, 98%, or 99% sequence identity thereto; or
the sequence set forth in SEQ ID NO:184, or an amino acid sequence that has at least 90%, 91%, 92%, 93%, 94%, 95%, 96%, 97%, 98%, or 99% sequence identity thereto, or a portion of any of the foregoing.

19. The DNA-targeting system of any of claims 1-18, wherein the regulatory DNA element is an enhancer.

20. The DNA-targeting system of any of claims 1-19, wherein the target site is located within the genomic coordinates human genome assembly GRCh38 (hg38) chr9:69,027,282-69,028,497, optionally wherein the target site is located within the genomic coordinates hg38 chr9:69,027,615-69,028,101.

21. A DNA-targeting system comprising:
(a) a DNA-targeting domain that binds to a target site in a regulatory DNA element of a frataxin (FXN) locus; and
(b) at least one effector domain that increases transcription of the FXN locus;
wherein the target site is located within the genomic coordinates human genome assembly GRCh38 (hg38) chr9:69,027,282-69,028,497.

22. The DNA-targeting system of any of claims 1-21, wherein the target site comprises the sequence set forth in SEQ ID NO:21, a contiguous portion thereof of at least 14 nt, or a complementary sequence of any of the foregoing.

23. The DNA-targeting system of any of claims 6-22, wherein the at least one gRNA comprises a gRNA spacer sequence comprising the sequence set forth in SEQ ID NO:42, or a contiguous portion thereof of at least 14 nt.

24. The DNA-targeting system of any of claims 6-23, wherein the at least one gRNA further comprises the sequence set forth in SEQ ID NO:44, and/or wherein the at least one gRNA comprises a gRNA that comprises the sequence set forth in SEQ ID NO:67, optionally wherein the at least one gRNA is the gRNA sequence set forth in SEQ ID NO:67.

25. The DNA-targeting system of any of claims 1, 5-11, and 15-18, wherein the regulatory DNA element is a promoter.

26. The DNA-targeting system of any of claims 1, 5-11, 15-18, and 25, wherein the target site is located within the genomic coordinates hg38 chr9:68,940,179-69,205,519.

27. The DNA-targeting system of any of claims 1, 5-11, 15-18, 25, and 26, wherein the target site comprises a sequence selected from any of SEQ ID NOS: 1-10, a contiguous portion thereof of at least 14 nt, or a complementary sequence of any of the foregoing.

28. The DNA-targeting system of any of claims 5-11, 15-18, and 25-27, wherein the at least one gRNA comprises a gRNA spacer sequence comprising a sequence selected from any of SEQ ID NOS:22-31, or a contiguous portion thereof of at least 14 nt.

29. The DNA-targeting system of any of claims 5-11, 15-18, and 25-28, wherein the at least one gRNA comprises a gRNA spacer sequence comprising SEQ ID NO:22, or a contiguous portion thereof of at least 14 nt.

30. The DNA-targeting system of any of claims 5-11, 15-18, and 25-28, wherein the at least one gRNA comprises a gRNA spacer sequence comprising SEQ ID NO:28, or a contiguous portion thereof of at least 14 nt.

31. The DNA-targeting system of any of claims 5-11, 15-18, and 25-30, wherein the gRNA further comprises the sequence set forth in SEQ ID NO:44, and/or wherein the at least one gRNA comprises a gRNA that comprises a sequence selected from any of SEQ ID NOS:47-56, optionally wherein the at least one gRNA is the gRNA sequence set forth in any of SEQ ID NOS:47-56, optionally wherein the gRNA is set forth in SEQ ID NO:47 or 53.

32. The DNA-targeting system of any of claims 5-11, 15-18, 25, and 26, wherein the target site comprises a sequence selected from any of SEQ ID NOS:11-20, a contiguous portion thereof of at least 14 nt, or a complementary sequence of any of the foregoing.

33. The DNA-targeting system of any of claims 5-11, 15-18, 25, 26, and 32, wherein the at least one gRNA comprises a gRNA spacer sequence comprising a sequence selected from any of SEQ ID NOS:32-41, or a contiguous portion thereof of at least 14 nt.

34. The DNA-targeting system of any of claims 5-11, 15-18, 25, 26, 32, and 33, wherein the gRNA further comprises the sequence set forth in SEQ ID NO:46, and/or wherein the at least one gRNA comprises a gRNA that comprises a sequence selected from any of SEQ ID NOS:57-66, optionally wherein the at least one gRNA is the gRNA sequence set forth in any of SEQ ID NOS:57-66.

35. The DNA-targeting system of any of claims 7-34, wherein the gRNA spacer sequence is between 14 nt and 24 nt, or between 16 nt and 22 nt in length, optionally wherein the gRNA spacer sequence is 18 nt, 19 nt, 20 nt, 21 nt or 22 nt in length.

36. The DNA-targeting system of any of claims 6-35, wherein the gRNA comprises modified nucleotides for increased stability.

37. The DNA-targeting system of any of claims 1-36, wherein the DNA-targeting domain or a component thereof is fused to the at least one effector domain, optionally wherein the DNA-targeting domain comprises a Cas-gRNA combination comprising (a) a Cas protein or a variant thereof and (b) at least one gRNA, and the component thereof fused to the at least one effector domain is the Cas protein or a variant thereof.

38. The DNA-targeting system of any of claims 1 and 3-37, wherein the effector domain induces transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation.

39. The DNA-targeting system of any of claims 1 and 3-38, wherein the effector domain induces transcription activation.

40. A DNA-targeting system comprising a DNA-targeting domain that is a Cas-guide RNA (gRNA) combination comprising:
(a) a Staphylococcus aureus deactivated Cas9 protein (dSaCas9) protein set forth in SEQ ID NO:72 fused to at least one effector domain that increases transcription of the FXN locus; and
(b) a gRNA comprising a gRNA spacer sequence set forth in SEQ ID NO:42.

41. A DNA-targeting system comprising a DNA-targeting domain that is a Cas-guide RNA (gRNA) combination comprising:
(a) a Staphylococcus aureus deactivated Cas9 protein (dSaCas9) protein set forth in SEQ ID NO:72 fused to at least one effector domain that increases transcription of the FXN locus; and
(b) a gRNA comprising a gRNA spacer sequence set forth in SEQ ID NO:22.

42. A DNA-targeting system comprising a DNA-targeting domain that is a Cas-guide RNA (gRNA) combination comprising:
(a) a Staphylococcus aureus deactivated Cas9 protein (dSaCas9) protein set forth in SEQ ID NO:72 fused to at least one effector domain that increases transcription of the FXN locus; and
(b) a gRNA comprising a gRNA spacer sequence set forth in SEQ ID NO:28.

43. The DNA-targeting system of any of claims 1 and 3-42, wherein the effector domain comprises at least one VP16 domain, or a VP16 tetramer (“VP64”) or a variant thereof, and/or wherein the effector domain comprises the sequence set forth in SEQ ID NO:81 or 83, or a portion thereof, or an amino acid sequence that has at least 90%, 91%, 92%, 93%, 94%, 95%, 96%, 97%, 98%, or 99% sequence identity to any of the foregoing.

44. The DNA-targeting system of any of claims 1 and 3-42, wherein the effector domain is selected from a p65 activation domain, a p300 domain, DPOLA, ENL, FOXO3, HSH2D, NCOA2, NCOA3, PSA1, PYGO1, RBM39, HERC2, DMD, or NOTCH2, or a domain thereof, a portion thereof or a variant thereof, optionally a truncation thereof, and/or wherein the effector domain comprises a sequence selected from any of SEQ ID NOS: 100-112, or a domain thereof, a portion thereof, or an amino acid sequence that has at least 90%, 91%, 92%, 93%, 94%, 95%, 96%, 97%, 98%, or 99% sequence identity to any of the foregoing.

45. The DNA-targeting system of any of claims 1 and 3-44, wherein the at least one effector domain is fused to the N-terminus, the C-terminus, or both the N-terminus and the C-terminus, of the DNA-targeting domain or a component thereof.

46. The DNA-targeting system of any of claims 1 and 3-45, further comprising one or more linkers connecting the DNA-targeting domain or a component thereof to the at least one effector domain, and/or further comprising one or more nuclear localization signals (NLS).

47. The DNA-targeting system of any of claims 1, 3-43, 45, and 46, wherein the DNA-targeting system comprises the sequence set forth in SEQ ID NO:71, or an amino acid sequence that has at least 90%, 91%, 92%, 93%, 94%, 95%, 96%, 97%, 98%, or 99% sequence identity thereto, or the sequence set forth in SEQ ID NO:77, or an amino acid sequence that has at least 90%, 91%, 92%, 93%, 94%, 95%, 96%, 97%, 98%, or 99% sequence identity thereto.

48. A combination, comprising:
a first DNA-targeting domain comprising the DNA targeting-domain of any of claims 1-47, and
one or more second DNA-targeting domains, optionally wherein the one or more second DNA-targeting domains comprises the DNA-targeting domain of any of claims 1-47.

49. The combination of claim 48, wherein:
the first DNA-targeting domain binds a first target site in an enhancer of a FXN locus; and
the second DNA-targeting domain binds a second target site in a promoter of a FXN locus.

50. The combination of claim 49, wherein:
the first target site is located within the genomic coordinates human genome assembly GRCh38 (hg38) chr9:69,027,282-69,028,497, optionally within the genomic coordinates hg38 chr9:69,027,615-69,028,101; and
the second target site is located within the genomic coordinates hg38 chr9:68,940,179-69,205,519;

51. The combination of claim 48, wherein:
the first DNA-targeting domain binds a first target site in a promoter of a FXN locus; and
the second DNA-targeting domain binds a second target site in a promoter of a FXN locus.

52. The combination of claim 48 or 51, wherein the first target site and the second target site independently are located within the genomic coordinates hg38 chr9:68,940,179-69,205,519, optionally wherein the first target site and the second target site are different.

53. The combination of any of claims 48, 51, and 52, wherein:
the first DNA-targeting domain comprises a first Cas-gRNA combination comprising (a) a first Cas protein or a variant thereof and (b) a first gRNA that is capable of hybridizing to the target site or is complementary to the first target site; and
the second DNA-targeting domain comprises a second Cas-gRNA combination comprising (a) a second Cas protein or a variant thereof and (b) a second gRNA that is capable of hybridizing to the target site or is complementary to the second target site.

54. The combination of any of claim 53, wherein:
the first Cas-gRNA combination comprises (a) a first Cas protein or a variant thereof and (b) a first gRNA comprising a gRNA spacer sequence set forth in SEQ ID NO:42 or a contiguous portion thereof of at least 14 nt; and
the second Cas-gRNA combination comprises (a) a second Cas protein or a variant thereof and (b) a second gRNA comprising a gRNA spacer sequence set forth in SEQ ID NO:22 or a contiguous portion thereof of at least 14 nt; or
the first Cas-gRNA combination comprises (a) a first Cas protein or a variant thereof and (b) a first gRNA comprising a gRNA spacer sequence set forth in SEQ ID NO:42 or a contiguous portion thereof of at least 14 nt; and
the second Cas-gRNA combination comprises (a) a second Cas protein or a variant thereof and (b) a second gRNA comprising a gRNA spacer sequence set forth in SEQ ID NO:28 or a contiguous portion thereof of at least 14 nt.

55. The combination of any of claim 53, wherein:
the first DNA-targeting domain comprises a first Cas-gRNA combination comprising (a) a first Cas protein or a variant thereof and (b) a first gRNA comprising a gRNA spacer sequence set forth in SEQ ID NO:22 or a contiguous portion thereof of at least 14 nt; and/or
the second DNA-targeting domain comprises a second Cas-gRNA combination comprising (a) a second Cas protein or a variant thereof and (b) a second gRNA comprising a gRNA spacer sequence set forth in SEQ ID NO:28 or a contiguous portion thereof of at least 14 nt.

56. The combination of any of claims 53-55, wherein the first Cas protein or a variant thereof and/or the second Cas protein or a variant thereof is a deactivated Cas9 (dCas9) protein, optionally a Staphylococcus aureus dCas9 (dSaCas9) protein or a Streptococcus pyogenes dCas9 (dS9Cas9) protein.

57. The combination of claim 56, wherein the first variant Cas protein and/or the second variant Cas protein is a Staphylococcus aureus dCas9 protein (dSaCas9) that comprises at least one amino acid mutation selected from D10A and N580A, with reference to numbering of positions of SEQ ID NO:73; or comprises the sequence set forth in SEQ ID NO:72, or an amino acid sequence that has at least 90%, 91%, 92%, 93%, 94%, 95%, 96%, 97%, 98%, or 99% sequence identity thereto.

58. The combination of claim 56 or 57, wherein the first variant Cas protein and/or the second variant Cas protein is a Streptococcus pyogenes dCas9 (dSpCas9) protein that comprises at least one amino acid mutation selected from D10A and H840A, with reference to numbering of positions of SEQ ID NO:79; or comprises the sequence set forth in SEQ ID NO:78, or an amino acid sequence that has at least 90%, 91%, 92%, 93%, 94%, 95%, 96%, 97%, 98%, or 99% sequence identity thereto.

59. The combination of any of claims 53-58, wherein the first variant Cas protein and/or the second variant Cas protein is a split variant Cas9 protein, wherein the split Cas9 protein comprises a first polypeptide comprising an N-terminal fragment of the variant Cas9 and an N-terminal Intein, and a second polypeptide comprising a C-terminal fragment of the variant Cas9 and a C-terminal Intein.

60. The combination of any of claims 53-59, wherein the first Cas protein and the second Cas protein are the same.

61. The combination of any of claims 53-59, wherein the first Cas protein and the second Cas protein are different.

62. The combination of any of claims 53-61, wherein the first Cas protein or a variant thereof and/or the second Cas protein or a variant thereof is fused to at least one effector domain, optionally wherein the effector domain induces transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation, optionally wherein the effector domain induces transcription activation.

63. The combination of any of claims 48-62, wherein the first Cas protein and the second Cas protein are encoded in a first polynucleotide and/or the first gRNA and the second gRNA are encoded in a first polynucleotide.

64. The combination of any of claims 48-62, wherein the first Cas protein is encoded in a first polynucleotide and the second Cas protein is encoded in a second polynucleotide; and/or wherein the first gRNA is encoded in a first polynucleotide and the second gRNA is encoded in a second polynucleotide, optionally wherein the first Cas protein and the first gRNA are encoded in a first polynucleotide, and the second Cas protein and the second gRNA are encoded in a second polynucleotide.

65. A guide RNA (gRNA) that binds a target site in an enhancer region of a frataxin (FXN) locus, wherein the target site is located within the genomic coordinates human genome assembly GRCh38 (hg38) chr9:69,027,282-69,028,497.

66. The gRNA of claim 65, wherein the target site is located within the genomic coordinates hg38 chr9:69,027,615-69,028,101.

67. The gRNA of any of claim 65 or 66, wherein:
the target site comprises the sequence set forth in SEQ ID NO:21, a contiguous portion thereof of at least 14 nt, or a complementary sequence of any of the foregoing;
the gRNA binds the sequence complementary to SEQ ID NO:21; and/or
the gRNA comprises a gRNA spacer sequence comprising the sequence set forth in SEQ ID NO:42, or a contiguous portion thereof of at least 14 nt.

68. The gRNA of any of claims 65-67, wherein the gRNA further comprises the sequence set forth in SEQ ID NO:44, optionally wherein the gRNA comprises the sequence set forth in SEQ ID NO:67, optionally wherein the gRNA is set forth in SEQ ID NO:67.

69. A guide RNA (gRNA) that binds a target site in a regulatory DNA element of a frataxin (FXN) locus wherein:
the target site comprises a sequence selected from any of SEQ ID NOS: 1-10, a contiguous portion thereof of at least 14 nt, or a complementary sequence of any of the foregoing; the gRNA binds the sequence complementary to any of SEQ ID NOS: 1-10; and/or the gRNA comprises a gRNA spacer sequence comprising a sequence selected from any of SEQ ID NOS:22-31, or a contiguous portion thereof of at least 14 nt.

70. The gRNA of claim 69, wherein the gRNA further comprises the sequence set forth in SEQ ID NO:44, optionally wherein the gRNA comprises a sequence selected from any of SEQ ID NOS:47-56, optionally wherein the gRNA is set forth in any of SEQ ID NOS:47-56, optionally wherein the gRNA is set forth in SEQ ID NO:47 or 53.

71. A guide RNA (gRNA) that binds a target site in a regulatory DNA element of a frataxin (FXN) locus wherein:
the target site comprises a sequence selected from any of SEQ ID NOS: 11-20, a contiguous portion thereof of at least 14 nt, or a complementary sequence of any of the foregoing;
the gRNA binds the sequence complementary to any of SEQ ID NOS: 11-20; and/or
the gRNA comprises a gRNA spacer sequence comprising a sequence selected from any of SEQ ID NOS:32-41, or a contiguous portion thereof of at least 14 nt.

72. The gRNA of claim 71, wherein:
the target site comprises a sequence selected from any of SEQ ID NOS: 12-14 and 16-19, a contiguous portion thereof of at least 14 nt, or a complementary sequence of any of the foregoing; and/or
the gRNA comprises a gRNA spacer sequence comprising a sequence selected from any of SEQ ID NOS:33-35 and 37-40, or a contiguous portion thereof of at least 14 nt.

73. The gRNA of claim 71 or 72, wherein the gRNA further comprises the sequence set forth in SEQ ID NO:46, optionally wherein the gRNA comprises a sequence selected from any of SEQ ID NOS:57-66, optionally wherein the gRNA is set forth in any of SEQ ID NOS:57-66.

74. The gRNA of any of claims 65-73, wherein the gRNA spacer sequence is between 14 nt and 24 nt, or between 16 nt and 22 nt in length, optionally wherein the gRNA spacer sequence is 18 nt, 19 nt, 20 nt, 21 nt or 22 nt in length.

75. The gRNA of any of claims 65-74, wherein the gRNA comprises modified nucleotides for increased stability.

76. The gRNA of any of claims 65-75, wherein the gRNA is capable of complexing with the Cas protein or variant thereof.

77. A combination, comprising a first gRNA comprising the gRNA of any of claims 65-76, and one or more second gRNAs that binds to a second target site in a regulatory DNA element of a frataxin (FXN) locus.

78. The combination of claim 77, wherein the second gRNA comprises the gRNA of any of claims 65-76.

79. A combination, comprising:
a first gRNA that binds a first target site in an enhancer region of a frataxin (FXN) locus, wherein the first target site is located within the genomic coordinates human genome assembly GRCh38 (hg38) chr9:69,027,282-69,028,497; and
a second gRNA that binds a second target site in a promoter region of a FXN locus, wherein the second target site is located within the genomic coordinates hg38 chr9:68,940,179-69,205,519.

80. The combination of claim 79, wherein:
the first gRNA comprises a gRNA spacer sequence set forth in SEQ ID NO:42 or a contiguous portion thereof of at least 14 nt; and
the second gRNA comprises a gRNA spacer sequence set forth in SEQ ID NO:22 or a contiguous portion thereof of at least 14 nt; or
the first gRNA comprises a gRNA spacer sequence set forth in SEQ ID NO:42 or a contiguous portion thereof of at least 14 nt; and
the second gRNA comprises a gRNA spacer sequence set forth in SEQ ID NO:28 or a contiguous portion thereof of at least 14 nt.

81. A combination, comprising:
a first gRNA that binds a first target site in a promoter region of a frataxin (FXN) locus, wherein the first target site is located within the genomic coordinates hg38 chr9:68,940,179-69,205,519; and
a second gRNA that binds a second target site in a promoter region of a FXN locus, wherein the second target site is located within the genomic coordinates hg38 chr9:68,940,179-69,205,519.

82. The combination of claim 81, wherein the combination comprises:
the first gRNA comprises a gRNA spacer sequence set forth in SEQ ID NO:22 or a contiguous portion thereof of at least 14 nt; and
the second gRNA comprises a gRNA spacer sequence set forth in SEQ ID NO:28 or a contiguous portion thereof of at least 14 nt.

83. A fusion protein comprising (1) a DNA-targeting domain or a component thereof and (2) at least one effector domain, wherein:
the DNA-targeting domain or a component thereof binds to a target site in a regulatory DNA element of a frataxin (FXN) locus; and
the effector domain increases transcription of the FXN locus.

84. The fusion protein of claim 83, wherein the DNA-targeting domain comprises a Clustered Regularly Interspaced Short Palindromic Repeats associated (Cas)-guide RNA (gRNA) combination comprising (a) a Cas protein or a variant thereof, and (b) at least one gRNA; a zinc finger protein (ZFP); a transcription activator-like effector (TALE); a meganuclease; a homing endonuclease; or an I-SceI enzyme or a variant thereof, optionally wherein the DNA-targeting domain comprises a catalytically inactive variant of any of the foregoing.

85. The fusion protein of claim 83 or 84, wherein the DNA-targeting domain comprises a Cas-gRNA combination comprising a Cas protein or a variant thereof and at least one gRNA, and the component of the DNA-targeting domain is a Cas protein or a variant thereof.

86. The fusion protein of claim 84 or 85, wherein the gRNA binds to a target site in a regulatory DNA element of a frataxin (FXN) locus.

87. A fusion protein comprising (1) a Cas protein or a variant thereof and (2) at least one effector domain, wherein the effector domain increases transcription of a FXN locus.

88. A fusion protein comprising (1) a first polypeptide of a split variant Cas protein comprising an N-terminal fragment of a Cas protein and an N-terminal Intein, and (2) at least one effector domain, wherein the effector domain increases transcription of a FXN locus.

89. A fusion protein comprising (1) a second polypeptide of a split variant Cas protein comprising a C-terminal fragment of a Cas protein and a C-terminal Intein and (2) at least one effector domain, wherein the effector domain increases transcription of the FXN locus.

90. The fusion protein of any of claims 84-89, wherein the Cas protein or a variant thereof is capable of complexing with at least one gRNA, optionally wherein the gRNA binds to a target site in a regulatory DNA element of a frataxin (FXN) locus.

91. The fusion protein of claim 83, wherein binding of the DNA-targeting domain or a component thereof to the target site does not introduce a genetic disruption or a DNA break at or near the target site.

92. The fusion protein of any of claims 84-91, wherein the Cas protein or a variant thereof is a deactivated Cas (dCas) protein.

93. The fusion protein of any of claims 84-92, wherein the Cas protein or a variant thereof is a deactivated Cas9 (dCas9) protein, optionally a Staphylococcus aureus dCas9 (dSaCas9) protein or a Streptococcus pyogenes dCas9 (dS9Cas9) protein.

94. A fusion protein comprising (1) a Staphylococcus aureus dCas9 protein (dSaCas9) and (2) at least one effector domain, wherein the effector domain increases transcription of a frataxin (FXN) locus.

95. The fusion protein of any of claims 84-94, wherein the Cas9 protein or a variant thereof is a Staphylococcus aureus dCas9 protein (dSaCas9) that comprises at least one amino acid mutation selected from D10A and N580A, with reference to numbering of positions of SEQ ID NO:73, and/or the Cas9 protein or a variant thereof comprises the sequence set forth in SEQ ID NO:72, or an amino acid sequence that has at least 90%, 91%, 92%, 93%, 94%, 95%, 96%, 97%, 98%, or 99% sequence identity thereto.

96. The fusion protein of any of claims 84-93, wherein the Cas9 protein or a variant thereof is a Streptococcus pyogenes dCas9 (dSpCas9) protein that comprises at least one amino acid mutation selected from D10A and H840A, with reference to numbering of positions of SEQ ID NO:79, and/or the Cas9 protein or a variant thereof comprises the sequence set forth in SEQ ID NO:78, or an amino acid sequence that has at least 90%, 91%, 92%, 93%, 94%, 95%, 96%, 97%, 98%, or 99% sequence identity thereto.

97. The fusion protein of any of claims 84-96, wherein the Cas protein or variant thereof is a split variant Cas protein, wherein the split variant Cas protein comprises a first polypeptide comprising an N-terminal fragment of the variant Cas protein and an N-terminal Intein, and a second polypeptide comprising a C-terminal fragment of the variant Cas protein and a C-terminal Intein, wherein when the first polypeptide and the second polypeptide of the split variant Cas protein are present in proximity or present in the same cell, the N-terminal Intein and C-terminal Intein self-excise and ligate the N-terminal fragment and the C-terminal fragment of the variant Cas protein to form a full-length variant Cas protein.

98. The fusion protein of claim 97, wherein:
the N-terminal Intein comprises an N-terminal Npu Intein, or the sequence set forth in SEQ ID NO:178, or an amino acid sequence that has at least 90%, 91%, 92%, 93%, 94%, 95%, 96%, 97%, 98%, or 99% sequence identity thereto, or a portion of any of the foregoing; and
the N-terminal fragment of the variant Cas protein comprises:
the N-terminal fragment of variant SpCas9 from the N-terminal end up to position 573 of the dSpCas9 sequence set forth in SEQ ID NO:78, or an amino acid sequence that has at least 90%, 91%, 92%, 93%, 94%, 95%, 96%, 97%, 98%, or 99% sequence identity thereto; or
the sequence set forth in SEQ ID NO:176, or an amino acid sequence that has at least 90%, 91%, 92%, 93%, 94%, 95%, 96%, 97%, 98%, or 99% sequence identity thereto, or a portion of any of the foregoing; and/or
wherein the C-terminal Intein comprises a C-terminal Npu Intein, or the sequence set forth in SEQ ID NO:182, or an amino acid sequence that has at least 90%, 91%, 92%, 93%, 94%, 95%, 96%, 97%, 98%, or 99% sequence identity thereto, or a portion of any of the foregoing; and
the C-terminal fragment of the variant Cas protein comprises:
the C-terminal fragment of variant SpCas9 from position 574 to the C-terminal end of the dSpCas9 sequence set forth in SEQ ID NO:78, or an amino acid sequence that has at least 90%, 91%, 92%, 93%, 94%, 95%, 96%, 97%, 98%, or 99% sequence identity thereto; or
the sequence set forth in SEQ ID NO:184, or an amino acid sequence that has at least 90%, 91%, 92%, 93%, 94%, 95%, 96%, 97%, 98%, or 99% sequence identity thereto, or a portion of any of the foregoing.

99. The fusion protein of any of claims 83-98, wherein the regulatory DNA element is an enhancer.

100. The fusion protein of any of claims 83-99, wherein the target site is located within the genomic coordinates human genome assembly GRCh38 (hg38) chr9:69,027,282-69,028,497, optionally wherein the target site is located within the genomic coordinates hg38 chr9:69,027,615-69,028,101.

101. The fusion protein of any of claims 83-100, wherein the target site comprises the sequence set forth in SEQ ID NO:21, a contiguous portion thereof of at least 14 nt, or a complementary sequence of any of the foregoing.

102. The fusion protein of any of claims 83-98, wherein the regulatory DNA element is a promoter, optionally wherein the target site is located within the genomic coordinates hg38 chr9:68,940,179-69,205,519.

103. The fusion protein of any of claims 83-98, and 102, wherein the target site comprises a sequence selected from any of SEQ ID NOS:1-10, a contiguous portion thereof of at least 14 nt, or a complementary sequence of any of the foregoing.

104. The fusion protein of any of claims 83-98, 102, and 103, wherein the target site comprises a sequence selected from any of SEQ ID NOS: 11-20, a contiguous portion thereof of at least 14 nt, or a complementary sequence of any of the foregoing.

105. The fusion protein of any of claims 83-104, wherein the effector domain induces transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, nuclease, signal transduction, proteolysis, ubiquitination, deubiquitination, phosphorylation, dephosphorylation, splicing, nucleic acid association, DNA methylation, DNA demethylation, histone methylation, histone demethylation, or DNA base oxidation.

106. The fusion protein of any of claims 83-105, wherein the effector domain induces transcription activation.

107. The fusion protein of any of claims 83-106, wherein the effector domain comprises at least one VP16 domain, or a VP16 tetramer (“VP64”) or a variant thereof, and/or wherein the effector domain comprises the sequence set forth in SEQ ID NO:81 or 83, or a portion thereof, or an amino acid sequence that has at least 90%, 91%, 92%, 93%, 94%, 95%, 96%, 97%, 98%, or 99% sequence identity to any of the foregoing.

108. The fusion protein of any of claims 83-106, wherein the effector domain is selected from a p65 activation domain, a p300 domain, DPOLA, ENL, FOXO3, HSH2D, NCOA2, NCOA3, PSA1, PYGO1, RBM39, HERC2, DMD, or NOTCH2, or a domain thereof, a portion thereof or a variant thereof, optionally a truncation thereof, and/or wherein the effector domain comprises a sequence selected from any of SEQ ID NOS: 100-112, or a domain thereof, a portion thereof, or an amino acid sequence that has at least 90%, 91%, 92%, 93%, 94%, 95%, 96%, 97%, 98%, or 99% sequence identity to any of the foregoing.

109. The fusion protein of any of claims 83-108, wherein the at least one effector domain is fused to the N-terminus, the C-terminus, or both the N-terminus and the C-terminus, of the DNA-targeting domain or a component thereof, optionally wherein the at least one effector domain is fused to the N-terminus, the C-terminus, or both the N-terminus and the C-terminus of the Cas protein or a variant thereof.

110. The fusion protein of any of claims 83-109, further comprising one or more linkers connecting the DNA-targeting domain or a component thereof, optionally the Cas protein or variant thereof, to the at least one effector domain, and/or further comprising one or more nuclear localization signals (NLS).

111. The fusion protein of any of claims 83-107, 109, and 110, wherein the fusion protein comprises the sequence set forth in SEQ ID NO:71, or an amino acid sequence that has at least 90%, 91%, 92%, 93%, 94%, 95%, 96%, 97%, 98%, or 99% sequence identity thereto, or comprises the sequence set forth in SEQ ID NO:77, or an amino acid sequence that has at least 90%, 91%, 92%, 93%, 94%, 95%, 96%, 97%, 98%, or 99% sequence identity thereto.

112. A combination comprising the fusion protein of any of claims 83-111 and at least one gRNA, optionally wherein the at least one gRNA is a gRNA of any of claims 118-151.

113. A polynucleotide encoding the DNA-targeting system of any of claims 1-47, the combination of any of claims 48-64, 77-82, and 112, the gRNA of any of claims 65-76, or the fusion protein of any of claims 83-111, or a portion or a component of any of the foregoing.

114. A polynucleotide encoding a first DNA-targeting system, a first Cas protein and/or a first gRNA of the combination of any of claims 48-64, 77-82, and 112.

115. A polynucleotide encoding a second DNA-targeting system, a second Cas protein and/or a second gRNA of the combination of any of claims 48-64, 77-82, and 112.

116. A plurality of polynucleotides, comprising the polynucleotide of any of claims 113-115, and one or more additional polynucleotides encoding an additional portion or an additional component of the DNA-targeting system of any of claims 1-47, the combination of any of claims 48-64, 77-82, and 112, the gRNA of any of claims 65-76, or the fusion protein of any of claims 83-111, or a portion or a component of any of the foregoing.

117. A plurality of polynucleotides, comprising:
a first polynucleotide comprising the polynucleotide of claim 114; and
a second polynucleotide comprising the polynucleotide of claim 115.

118. A vector comprising the polynucleotide of any of claims 113-115, the plurality of polynucleotides of claim 116 or 117, or a first polynucleotide or a second polynucleotide of the plurality of polynucleotides of claim 116 or 117, or a portion or a component of any of the foregoing.

119. The vector of claim 118, wherein the vector is a viral vector, optionally wherein the viral vector is an AAV vector.

120. The vector of claim 119, wherein the viral vector, optionally the AAV vector, exhibits tropism for a nervous system cell, optionally a neuron, a heart cell, optionally a cardiomyocyte, a skeletal muscle cell, a fibroblast, an induced pluripotent stem cell, or a cell derived from any of the foregoing; and/or wherein the viral vector is an AAV vector and the AAV vector is selected from among AAV1, AAV2, AAV3, AAV4, AAV5, AAV6, AAV7, AAV8, AAV9, AAV10, AAV11, AAV12, or AAV-DJ vector, optionally an AAV9 vector.

121. The vector of claim 118, wherein the vector is a non-viral vector selected from a lipid nanoparticle, a liposome, an exosome, or a cell penetrating peptide

122. A plurality of vectors, comprising the vector of any of claims 118-121, and one or more additional vectors comprising one or more additional polynucleotides encoding an additional portion or an additional component of the DNA-targeting system of any of claims 1-47, the combination of any of claims 48-64, 77-82, and 112, the gRNA of any of claims 65-76, or the fusion protein of any of claims 83-111, or a portion or a component of any of the foregoing.

123. A plurality of vectors, comprising:
a first vector comprising the polynucleotide of claim 114; and
a second vector comprising the polynucleotide of claim 115.

124. A cell comprising the DNA-targeting system of any of claims 1-47, the combination of any of claims 48-64, 77-82, and 112, the gRNA of any of claims 65-76, the fusion protein of any of claims 83-111, the polynucleotide of any of claims 113-115, the plurality of polynucleotides of claim 116 or 117, the vector of any of claims 118-121, the plurality of vectors of claim 122 or 123, or a portion or a component of any of the foregoing.

125. The cell of claim 124, wherein the cell is a nervous system cell, optionally a neuron, a heart cell, optionally a cardiomyocyte, a skeletal muscle cell, a fibroblast, an induced pluripotent stem cell, or a cell derived from any of the foregoing, optionally wherein the cell is from a subject that has or is suspected of having Friedreich's ataxia (FA).

126. A pharmaceutical composition comprising the DNA-targeting system of any of claims 1-47, the combination of any of claims 48-64, 77-82, and 112, the gRNA of any of claims 65-76, the fusion protein of any of claims 83-111, the polynucleotide of any of claims 113-115, the plurality of polynucleotides of claim 116 or 117, the vector of any of claims 118-121, the plurality of vectors of claim 122 or 123, or a portion or a component of any of the foregoing.

127. A method for modulating the expression of frataxin (FXN) in a cell, the method comprising:
introducing the DNA-targeting system of any of claims 1-47, the combination of any of claims 48-64, 77-82, and 112, the gRNA of any of claims 65-76, the fusion protein of any of claims 83-111, the polynucleotide of any of claims 113-115, the plurality of polynucleotides of claim 116 or 117, the vector of any of claims 118-121, the plurality of vectors of claim 122 or 123, the pharmaceutical composition of claim 126, or a portion or a component of any of the foregoing, into the cell.

128. A method of inducing a genetic disruption at a target site in an enhancer region of a frataxin (FXN) locus in a cell, wherein the target site is located within the genomic coordinates human genome assembly GRCh38 (hg38) chr9:69,027,282-69,028,497, the method comprising:
contacting a cell with the DNA-targeting system of the DNA-targeting system of any of claims 1-47, the combination of any of claims 48-64, 77-82, and 112, the gRNA of any of claims 65-76, the fusion protein of any of claims 83-111, the polynucleotide of any of claims 113-115, the plurality of polynucleotides of claim 116 or 117, the vector of any of claims 118-121, the plurality of vectors of claim 122 or 123, the pharmaceutical composition of claim 126, or a portion or a component of any of the foregoing.

129. The method of claim 127 or 128, wherein the cell is from a subject that has or is suspected of having Friedreich's ataxia (FA), optionally wherein the subject has or is suspected of having Friedreich's ataxia (FA).

130. A method for modulating the expression of frataxin (FXN) in a subject, the method comprising:
administering the DNA-targeting system of any of claims 1-47, the combination of any of claims 48-64, 77-82, and 112, the gRNA of any of claims 65-76, the fusion protein of any of claims 83-111, the polynucleotide of any of claims 113-115, the plurality of polynucleotides of claim 116 or 117, the vector of any of claims 118-121, the plurality of vectors of claim 122 or 123, the pharmaceutical composition of claim 126, or a portion or a component of any of the foregoing, to the subject.

131. The method of claim 129 or 130, wherein the subject has or is suspected of having Friedreich's ataxia (FA).

132. A method of treating Friedreich's ataxia (FA), the method comprising:
administering the DNA-targeting system of any of claims 1-47, the combination of any of claims 48-64, 77-82, and 112, the gRNA of any of claims 65-76, the fusion protein of any of claims 83-111, the polynucleotide of any of claims 113-115, the plurality of polynucleotides of claim 116 or 117, the vector of any of claims 118-121, the plurality of vectors of claim 122 or 123, the pharmaceutical composition of claim 126, or a portion or a component of any of the foregoing, to a subject that has or is suspected of having FA.

133. The method of any of claims 129-132, wherein:
a cell in the subject exhibits reduced expression of FXN compared to a cell from a normal subject; and/or
a cell in the subject has a GAA trinucleotide repeat expansion in the FXN gene.

134. The method of any of claims 127-133, wherein the cell is a nervous system cell, optionally a neuron, a heart cell, optionally a cardiomyocyte, a skeletal muscle cell, a fibroblast, an induced pluripotent stem cell, or a cell derived from any of the foregoing.

135. The method of any of claims 127-134, wherein the introducing, contacting or administering is carried out in vivo or ex vivo.

136. The method of any of claims 127-135, wherein following the introducing, contacting or administering, the expression of frataxin (FXN) is increased in the cell or the subject, optionally wherein:
the expression is increased at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold; and/or
the expression is increased by less than about 10-fold, 9-fold, 8-fold, 7-fold or 6-fold.

137. The method of any of claims 129-136, wherein the subject is a human.
